SEQUENCE LISTING (92 sequences)

SEQ ID NO: 1 - protein, 17 aa, Artificial Sequence
  Synthetic Construct
  VARIANT 1: Xaa = beta-Ala or Ser
  VARIANT 2, 6: Xaa = Lys, Arg, or Lys
  VARIANT 3, 7, 11: Xaa = Phe or Trp
  VARIANT 4, 8: Xaa = Phe, Trp, or Tyr
  VARIANT 5, 7, 10: Xaa = Glu, Arg, or Ser
  VARIANT 9: Xaa = Arg, Thr, or Ser
  VARIANT 12: Xaa = Phe or Trp, and can be absent
  VARIANT 13: Xaa = Pro or Arg
  VARIANT 14: Xaa = Arg or Leu
  VARIANT 15: Xaa = Lys, Trp, or Arg
  VARIANT 16: Xaa = Arg or Phe
  VARIANT 17: Xaa = Arg or Lys
XXXXXXXXXXXXXXXXX

SEQ ID NO: 2 - protein, 16 aa, Artificial Sequence
  Synthetic Construct
  VARIANT 1: Xaa = beta-Ala or Ser
  VARIANT 2, 6, 14, 16: Xaa = Arg or Lys
  VARIANT 4, 8: Xaa = Phe, Trp, or Tyr
  VARIANT 9: Xaa = Thr or Arg
  VARIANT 10: Xaa = Glu or Arg
  VARIANT 11: Xaa = Trp or Phe
XXWXEXWXXXXPRXRX

SEQ ID NO: 3 - protein, 16 aa, Artificial Sequence
  Synthetic Construct
  VARIANT 1: Xaa = beta-Ala or Ser
XKWFERWFREWPRKRR

SEQ ID NO: 4 - protein, 16 aa, Artificial Sequence
  Synthetic Construct
  VARIANT 1: Xaa = beta-Ala or Ser
XKWWERWWREWPRKRR

SEQ ID NO: 5 - protein, 16 aa, Artificial Sequence
  Synthetic Construct
  VARIANT 1: Xaa = beta-Ala or Ser
XKWWERWWREWPRKRK

SEQ ID NO: 6 - protein, 16 aa, Artificial Sequence
  Synthetic Construct
  VARIANT 1: Xaa = beta-Ala or Ser
XRWWEKWWTRWPRKRK

SEQ ID NO: 7 - protein, 16 aa, Artificial Sequence
  Synthetic Construct
  VARIANT 1: Xaa = beta-Ala or Ser
XRWYEKWYTEFPRRRR

SEQ ID NO: 8 - protein, 17 aa, Artificial Sequence
  Synthetic Construct
  VARIANT 1: Xaa = beta-Ala or Ser
  VARIANT 3, 11: Xaa = Any non-natural amino acid
  VARIANT 3, 11: Linked by hydrocarbon linkage
XKXWWERWWRXWPRKRK

SEQ ID NO: 9 - protein, 17 aa, Artificial Sequence
  Synthetic Construct
  VARIANT 1: Xaa = beta-Ala or Ser
  VARIANT 2: Xaa = Lys, Arg, or Leu
  VARIANT 3, 7, 12: Xaa = Phe or Trp
  VARIANT 5, 9, 10: Xaa = Arg or Ser
  VARIANT 6, 14: Xaa = Leu or Arg
  VARIANT 13: Xaa = Arg or Pro
  VARIANT 16: Xaa = Arg or Phe
XXXWXXXWXXWXXXWXR

SEQ ID NO: 10 - protein, 17 aa, Artificial Sequence
  Synthetic Construct
  VARIANT 1: Xaa = beta-Ala or Ser
XRWWRLWWRSWFRLWRR

SEQ ID NO: 11 - protein, 17 aa, Artificial Sequence
  Synthetic Construct
  VARIANT 1: Xaa = beta-Ala or Ser
XLWWRRWWSRWWPRWRR

SEQ ID NO: 12 - protein, 17 aa, Artificial Sequence
  Synthetic Construct
  VARIANT 1: Xaa = beta-Ala or Ser
XLWWSRWWRSWFRLWFR

SEQ ID NO: 13 - protein, 17 aa, Artificial Sequence
  Synthetic Construct
  VARIANT 1: Xaa = beta-Ala or Ser
XKFWSRFWRSWFRLWRR

SEQ ID NO: 14 - protein, 17 aa, Artificial Sequence
  Synthetic Construct
  VARIANT 1: Xaa = beta-Ala or Ser
  VARIANT 5, 12: Xaa = Any non-natural amino acid
  VARIANT 5, 12: Linked by a hydrocarbon linkage
XRWWXLWWRSWXRLWRR

SEQ ID NO: 15 - protein, 20 aa, Artificial Sequence
  Synthetic Construct
  VARIANT 1: Xaa = beta-Ala or Ser
  VARIANT 3: Xaa = Phe or Trp
  VARIANT 8, 11, 15: Xaa = Arg or Ser
  VARIANT 10: Xaa = Leu, Trp, Cys, or Ile
  VARIANT 12: Xaa = Ser, Ala, Asn, or Thr
  VARIANT 17: Xaa = Leu or Trp
  VARIANT 18: Xaa = Trp or Arg
  VARIANT 19: Xaa = Lys or Arg
  VARIANT 20: Xaa = Ala and can be absent
XLXRALWXLXXXLWXLXXXX

SEQ ID NO: 16 - protein, 20 aa, Artificial Sequence
  Synthetic Construct
  VARIANT 1: Xaa = beta-Ala or Ser
  VARIANT 3: Xaa = Phe or Trp
  VARIANT 8, 11, 15: Xaa = Arg or Ser
  VARIANT 10: Xaa = Leu, Trp, Cys, or Ile
  VARIANT 12: Xaa = Ser, Ala, Asn, or Thr
  VARIANT 17: Xaa = Leu or Trp
  VARIANT 18: Xaa = Trp or Arg
  VARIANT 19: Xaa = Lys or Arg
  VARIANT 20: Xaa = Ala and can be absent
XLXLARWXLXXXLWXLXXXX

SEQ ID NO: 17 - protein, 20 aa, Artificial Sequence
  Synthetic Construct
  VARIANT 1: Xaa = beta-Ala or Ser
  VARIANT 3: Xaa = Phe or Trp
  VARIANT 8, 11, 15: Xaa = Arg or Ser
  VARIANT 10: Xaa = Leu, Trp, Cys, or Ile
  VARIANT 12: Xaa = Ser, Ala, Asn, or Thr
  VARIANT 17: Xaa = Leu or Trp
  VARIANT 18: Xaa = Trp or Arg
  VARIANT 19: Xaa = Lys or Arg
  VARIANT 20: Xaa = Ala and can be absent
XLXARLWXLXXXLWXLXXXX

SEQ ID NO: 18 - protein, 20 aa, Artificial Sequence
  Synthetic Construct
  VARIANT 1: Xaa = beta-Ala or Ser
  VARIANT 3: Xaa = Phe or Trp
  VARIANT 10: Xaa = Leu, Trp, Cys, or Ile
  VARIANT 12: Xaa = Ser, Ala, Asn, or Thr
  VARIANT 17: Xaa = Leu or Trp
  VARIANT 18: Xaa = Trp or Arg
  VARIANT 19: Xaa = Lys or Arg
  VARIANT 20: Xaa = Ala and can be absent
XLXRALWRLXRXLWRLXXXX

SEQ ID NO: 19 - protein, 20 aa, Artificial Sequence
  Synthetic Construct
  VARIANT 1: Xaa = beta-Ala or Ser
  VARIANT 3: Xaa = Phe or Trp
  VARIANT 10, 17: Xaa = Leu or Trp
  VARIANT 12: Xaa = Ser, Ala, or Asn
  VARIANT 18: Xaa = Trp or Arg
  VARIANT 20: Xaa = Ala and can be absent
XLXRALWRLXRXLWRLXXKX

SEQ ID NO: 20 - protein, 19 aa, Artificial Sequence
  Synthetic Construct
  VARIANT 1: Xaa = beta-Ala or Ser
  VARIANT 12: Xaa = Ser or Thr
  VARIANT 19: Xaa = Lys or Arg
XLFRALWRLLRXLWRLLWX

SEQ ID NO: 21 - protein, 20 aa, Artificial Sequence
  Synthetic Construct
  VARIANT 1: Xaa = beta-Ala or Ser
  VARIANT 12: Xaa = Ser or Thr
  VARIANT 19: Xaa = Lys or Arg
XLWRALWRLWRXLWRLLWXA

SEQ ID NO: 22 - protein, 20 aa, Artificial Sequence
  Synthetic Construct
  VARIANT 1: Xaa = beta-Ala or Ser
  VARIANT 10: Xaa = Leu, Cys, or Ile
  VARIANT 12: Xaa = Ser or Thr
  VARIANT 19: Xaa = Lys or Arg
XLWRALWRLXRXLWRLWRXA

SEQ ID NO: 23 - protein, 20 aa, Artificial Sequence
  Synthetic Construct
  VARIANT 1: Xaa = beta-Ala or Ser
  VARIANT 12: Xaa = Ser or Thr
  VARIANT 19: Xaa = Lys or Arg
XLWRALWRLWRXLWRLWRXA

SEQ ID NO: 24 - protein, 20 aa, Artificial Sequence
  Synthetic Construct
  VARIANT 1: Xaa = beta-Ala or Ser
  VARIANT 10: Xaa = Leu or Ile
  VARIANT 19: Xaa = Lys or Arg
XLWRALWRLXRALWRLLWXA

SEQ ID NO: 25 - protein, 20 aa, Artificial Sequence
  Synthetic Construct
  VARIANT 1: Xaa = beta-Ala or Ser
  VARIANT 10: Xaa = Leu, Cys, or Ile
  VARIANT 19: Xaa = Lys or Arg
XLWRALWRLXRNLWRLLWXA

SEQ ID NO: 26 - protein, 19 aa, Artificial Sequence
  Synthetic Construct
  VARIANT 1: Xaa = beta-Ala or Ser
XLFRALWRLLRSLWRLLWK

SEQ ID NO: 27 - protein, 20 aa, Artificial Sequence
  Synthetic Construct
  VARIANT 1: Xaa = beta-Ala or Ser
XLWRALWRLWRSLWRLLWKA

SEQ ID NO: 28 - protein, 20 aa, Artificial Sequence
  Synthetic Construct
  VARIANT 1: Xaa = beta-Ala or Ser
XLWRALWRLLRSLWRLWRKA

SEQ ID NO: 29 - protein, 20 aa, Artificial Sequence
  Synthetic Construct
  VARIANT 1: Xaa = beta-Ala or Ser
XLWRALWRLWRSLWRLWRKA

SEQ ID NO: 30 - protein, 20 aa, Artificial Sequence
  Synthetic Construct
  VARIANT 1: Xaa = beta-Ala or Ser
XLWRALWRLLRALWRLLWKA

SEQ ID NO: 31 - protein, 20 aa, Artificial Sequence
  Synthetic Construct
  VARIANT 1: Xaa = beta-Ala or Ser
XLWRALWRLLRNLWRLLWKA

SEQ ID NO: 32 - protein, 19 aa, Artificial Sequence
  Synthetic Construct
  VARIANT 1: Xaa = beta-Ala or Ser
  VARIANT 8, 12: Linked by a hydrocarbon linkage
XLFRALWRLLRSLWRLLWK

SEQ ID NO: 33 - protein, 19 aa, Artificial Sequence
  Synthetic Construct
  VARIANT 1: Xaa = beta-Ala or Ser
  VARIANT 8, 12: Linked by a hydrocarbon linkage
XLFLARWRLLRSLWRLLWK

SEQ ID NO: 34 - protein, 19 aa, Artificial Sequence
  Synthetic Construct
  VARIANT 1: Xaa = beta-Ala or Ser
  VARIANT 8, 12: Linked by a hydrocarbon linkage
XLFRALWSLLRSLWRLLWK

SEQ ID NO: 35 - protein, 19 aa, Artificial Sequence
  Synthetic Construct
  VARIANT 1: Xaa = beta-Ala or Ser
  VARIANT 8, 12: Linked by a hydrocarbon linkage
XLFLARWSLLRSLWRLLWK

SEQ ID NO: 36 - protein, 19 aa, Artificial Sequence
  Synthetic Construct
  VARIANT 1: Xaa = beta-Ala or Ser
  VARIANT 11, 15: Linked by a hydrocarbon linkage
XLFRALWRLLRSLWSLLWK

SEQ ID NO: 37 - protein, 19 aa, Artificial Sequence
  Synthetic Construct
  VARIANT 1: Xaa = beta-Ala or Ser
  VARIANT 11, 15: Linked by a hydrocarbon linkage
XLFLARWRLLRSLWSLLWK

SEQ ID NO: 38 - protein, 19 aa, Artificial Sequence
  Synthetic Construct
  VARIANT 1: Xaa = beta-Ala or Ser
  VARIANT 11, 15: Linked by a hydrocarbon linkage
XLFRALWRLLSSLWSLLWK

SEQ ID NO: 39 - protein, 19 aa, Artificial Sequence
  Synthetic Construct
  VARIANT 1: Xaa = beta-Ala or Ser
  VARIANT 11, 15: Linked by a hydrocarbon linkage
XLFLARWRLLSSLWSLLWK

SEQ ID NO: 40 - protein, 19 aa, Artificial Sequence
  Synthetic Construct
  VARIANT 1: Xaa = beta-Ala or Ser
  VARIANT 5, 12: Linked by a hydrocarbon linkage
XLFARLWRLLRSLWRLLWK

SEQ ID NO: 41 - protein, 20 aa, Artificial Sequence
  Synthetic Construct
  VARIANT 1: Xaa = beta-Ala or Ser
  VARIANT 2: Xaa = Leu and can be absent
  VARIANT 3, 11: Xaa = Arg and can be absent
  VARIANT 6: Xaa = Leu, Arg, or Gly
  VARIANT 7: Xaa = Arg, Trp, or Ser
  VARIANT 10: Xaa = Ser, Pro, or Thr
  VARIANT 12: Xaa = Trp or Pro
  VARIANT 13: Xaa = Phe, Ala, or Arg
  VARIANT 14: Xaa = Ser, Leu, Pro, or Arg
  VARIANT 15: Xaa = Arg or Ser
  VARIANT 16: Xaa = Trp and can be absent
  VARIANT 17: Xaa = Ala or Arg, and can be absent
  VARIANT 19: Xaa = Trp or Phe
  VARIANT 3, 11, 2, 16, 17: Wherein if positions 3 and 11 are absent, then positions 2, 16, and 17 are absent as well
XXXWWXXWAXXXXXXXXWXR

SEQ ID NO: 42 - protein, 20 aa, Artificial Sequence
  Synthetic Construct
  VARIANT 1: Xaa = beta-Ala or Ser
  VARIANT 2: Xaa = Leu and can be absent
  VARIANT 10: Xaa = Ser or Pro
  VARIANT 13: Xaa = Phe or Ala
  VARIANT 14: Xaa = Ser, Leu, or Pro
  VARIANT 15: Xaa = Arg or Ser
  VARIANT 17: Xaa = Ala or Arg
  VARIANT 19: Xaa = Trp or Phe
XXRWWLRWAXRWXXXWXWXR

SEQ ID NO: 43 - protein, 20 aa, Artificial Sequence
  Synthetic Construct
  VARIANT 1: Xaa = beta-Ala or Ser
XLRWWLRWASRWFSRWAWWR

SEQ ID NO: 44 - protein, 20 aa, Artificial Sequence
  Synthetic Construct
  VARIANT 1: Xaa = beta-Ala or Ser
XLRWWLRWASRWASRWAWFR

SEQ ID NO: 45 - protein, 19 aa, Artificial Sequence
  Synthetic Construct
  VARIANT 1: Xaa = beta-Ala or Ser
XRWWLRWASRWALSWRWWR

SEQ ID NO: 46 - protein, 19 aa, Artificial Sequence
  Synthetic Construct
  VARIANT 1: Xaa = beta-Ala or Ser
XRWWLRWASRWFLSWRWWR

SEQ ID NO: 47 - protein, 19 aa, Artificial Sequence
  Synthetic Construct
  VARIANT 1: Xaa = beta-Ala or Ser
XRWWLRWAPRWFPSWRWWR

SEQ ID NO: 48 - protein, 19 aa, Artificial Sequence
  Synthetic Construct
  VARIANT 1: Xaa = beta-Ala or Ser
XRWWLRWASRWAPSWRWWR

SEQ ID NO: 49 - protein, 15 aa, Artificial Sequence
  Synthetic Construct
  VARIANT 1: Xaa = beta-Ala or Ser
  VARIANT 4: Xaa = Arg or Gly
  VARIANT 5: Xaa = Trp or Ser
  VARIANT 8: Xaa = Ser, Thr, or Pro
  VARIANT 9: Xaa = Trp or Pro
  VARIANT 10: Xaa = Ala or Arg
  VARIANT 12: Xaa = Ser or Arg
XWWXXWAXXXRXWWR

SEQ ID NO: 50 - protein, 15 aa, Artificial Sequence
  Synthetic Construct
  VARIANT 1: Xaa = beta-Ala or Ser
XWWRWWASWARSWWR

SEQ ID NO: 51 - protein, 15 aa, Artificial Sequence
  Synthetic Construct
  VARIANT 1: Xaa = beta-Ala or Ser
XWWGSWATPRRRWWR

SEQ ID NO: 52 - protein, 15 aa, Artificial Sequence
  Synthetic Construct
  VARIANT 1: Xaa = beta-Ala or Ser
XWWRWWAPWARSWWR

SEQ ID NO: 53 - protein, 20 aa, Artificial Sequence
  Synthetic Construct
  VARIANT 1: Xaa = Any Amino Acid and can be absent
  VARIANT 6, 7, 8, 15, 16, 17, 18: Xaa = Any Amino Acid
XKWRSXXXRWRLWRXXXXSR

SEQ ID NO: 54 - protein, 20 aa, Artificial Sequence
  Synthetic Construct
  VARIANT 1: Xaa = beta-Ala or Ser, and can be absent
  VARIANT 6: Xaa = Ala or Val
  VARIANT 7: Xaa = Gly or Leu
  VARIANT 8, 18: Xaa = Trp or Tyr
  VARIANT 15: Xaa = Val or Ser
  VARIANT 16: Xaa = Arg, Val, or Ala
  VARIANT 17: Xaa = Ser or Leu
XKWRSXXXRWRLWRXXXXSR

SEQ ID NO: 55 - protein, 19 aa, Artificial Sequence
  Synthetic Construct
KWRSAGWRWRLWRVRSWSR

SEQ ID NO: 56 - protein, 19 aa, Artificial Sequence
  Synthetic Construct
KWRSALYRWRLWRVRSWSR

SEQ ID NO: 57 - protein, 19 aa, Artificial Sequence
  Synthetic Construct
KWRSALYRWRLWRSRSWSR

SEQ ID NO: 58 - protein, 19 aa, Artificial Sequence
  Synthetic Construct
KWRSALYRWRLWRSALYSR

SEQ ID NO: 59 - protein, 19 aa, Artificial Sequence
  Synthetic Construct
  VARIANT 4, 8: Linked by a hydrocarbon linkage
KWRSAGWRWRLWRVRSWSR

SEQ ID NO: 60 - protein, 19 aa, Artificial Sequence
  Synthetic Construct
  VARIANT 3, 10: Linked by a hydrocarbon linkage
KWRSAGWRWRLWRVRSWSR

SEQ ID NO: 61 - protein, 19 aa, Artificial Sequence
  Synthetic Construct
  VARIANT 8, 15: Linked by a hydrocarbon linkage
KWRSAGWRWRLWRVRSWSR

SEQ ID NO: 62 - protein, 19 aa, Artificial Sequence
  Synthetic Construct
  VARIANT 4, 8: Linked by a hydrocarbon linkage
KWRSALYRWRLWRSRSWSR

SEQ ID NO: 63 - protein, 19 aa, Artificial Sequence
  Synthetic Construct
  VARIANT 3, 10: Linked by a hydrocarbon linkage
KWRSALYRWRLWRSRSWSR

SEQ ID NO: 64 - protein, 19 aa, Artificial Sequence
  Synthetic Construct
  VARIANT 8, 15: Linked by a hydrocarbon linkage
KWRSALYRWRLWRSRSWSR

SEQ ID NO: 65 - protein, 19 aa, Artificial Sequence
  Synthetic Construct
  VARIANT 10, 14: Linked by a hydrocarbon linkage
KWRSALYRWRLWRSRSWSR

SEQ ID NO: 66 - protein, 19 aa, Artificial Sequence
  Synthetic Construct
  VARIANT 14, 18: Linked by a hydrocarbon linkage
KWRSALYRWRLWRSRSWSR

SEQ ID NO: 67 - protein, 19 aa, Artificial Sequence
  Synthetic Construct
  VARIANT 3, 10: Linked by a hydrocarbon linkage
KWRSALYRWRLWRSALYSR

SEQ ID NO: 68 - protein, 19 aa, Artificial Sequence
  Synthetic Construct
  VARIANT 4, 8: Linked by a hydrocarbon linkage
KWRSALYRWRLWRSALYSR

SEQ ID NO: 69 - protein, 19 aa, Artificial Sequence
  Synthetic Construct
  VARIANT 10, 14: Linked by a hydrocarbon linkage
KWRSALYRWRLWRSALYSR

SEQ ID NO: 70 - protein, 19 aa, Artificial Sequence
  Synthetic Construct
  VARIANT 14, 18: Linked by a hydrocarbon linkage
KWRSALYRWRLWRSALYSR

SEQ ID NO: 71 - protein, 21 aa, Artificial Sequence
  Synthetic Construct
KETWWETWWTEWSQPKKKRKV

SEQ ID NO: 72 - protein, 21 aa, Artificial Sequence
  Synthetic Construct
KETWFETWFTEWSQPKKKRKV

SEQ ID NO: 73 - protein, 15 aa, Artificial Sequence
  Synthetic Construct
KWFETWFTEWPKKRK

SEQ ID NO: 74 - protein, 27 aa, Artificial Sequence
  Synthetic Construct
GALFLGFLGAAGSTMGAWSQPKKKRKV

SEQ ID NO: 75 - protein, 16 aa, Artificial Sequence
  Synthetic Construct
  VARIANT 1: beta-Ala
AKWFERWFREWPRKRR

SEQ ID NO: 76 - protein, 16 aa, Artificial Sequence
  Synthetic Construct
  VARIANT 1: beta-Ala
AKWWERWWREWPRKRR

SEQ ID NO: 77 - protein, 20 aa, Artificial Sequence
  Synthetic Construct
  VARIANT 1: beta-Ala
ALWRALWRLWRSLWRLLWKA

SEQ ID NO: 78 - protein, 20 aa, Artificial Sequence
  Synthetic Construct
  VARIANT 1: beta-Ala
ALRWWLRWASRWFSRWAWWR

SEQ ID NO: 79 - protein, 20 aa, Artificial Sequence
  Synthetic Construct
  VARIANT 1: beta-Ala
AKWRSAGWRWRLWRVRSWSR

SEQ ID NO: 80 - protein, 20 aa, Artificial Sequence
  Synthetic Construct
  VARIANT 1: beta-Ala
AKWRSALYRWRLWRVRSWSR

SEQ ID NO: 81 - protein, 20 aa, Artificial Sequence
  Synthetic Construct
GLWRALWRLLRSLWRLLWKV

SEQ ID NO: 82 - DNA, 20 bp, Artificial Sequence
  Synthetic Construct
acaactttaccgaccgcgcc

SEQ ID NO: 83 - DNA, 21 bp, Artificial Sequence
  Synthetic Construct
guuggagcuuguggcguagtt

SEQ ID NO: 84 - DNA, 21 bp, Artificial Sequence
  Synthetic Construct
cuacgccaccagcuccaactt

SEQ ID NO: 85 - DNA, 22 bp, Artificial Sequence
  Synthetic Construct
gatgaggctattcatgatgatt

SEQ ID NO: 86 - DNA, 21 bp, Artificial Sequence
  Synthetic Construct
gaagugcauacaccgagactt

SEQ ID NO: 87 - DNA, 20 bp, Artificial Sequence
  Synthetic Construct
gucucgguguagcacuuctt

SEQ ID NO: 88 - DNA, 21 bp, Artificial Sequence
  Synthetic Construct
guuggagcuguuggcguagtt

SEQ ID NO: 89 - DNA, 21 bp, Artificial Sequence
  Synthetic Construct
cuacgccaacagcuccaactt

SEQ ID NO: 90 - DNA, 20 bp, Artificial Sequence
  Synthetic Construct
atgaagcacctgaacaccgt

SEQ ID NO: 91 - DNA, 9 bp, Artificial Sequence
  Synthetic Construct
  misc_feature 3: n = A or G
ccnccaugg

SEQ ID NO: 92 - DNA, 13 bp, Artificial Sequence
  Synthetic Construct
  misc_feature 7: n = A or G
gccgccnccaugg

(19) United States
(12) Patent Application Publication
(10) Pub. No.: US 2020/0323964 A1
(43) Pub. Date: Oct. 15, 2020

(54) PEPTIDES AND NANOPARTICLES FOR INTRACELLULAR DELIVERY OF MRNA

(71) Applicant: AADIGEN, LLC, Pacific Palisades, CA (US)

(72) Inventors: Neil DESAI, Pacific Palisades, CA (US); Gilles DIVITA, St Andre De Sangonis (FR)

(21) Appl. No.: 16/756,442

(22) PCT Filed: Oct. 15, 2018
(86) PCT No.: PCT/US2018/055955

§ 371 Date: Apr. 15, 2020

(30) Foreign Application Priority Data
Oct. 16, 2017 (FR) 17/59645
Apr. 17, 2018 (FR) 18/53370

(51) Int. Cl. A61K 38/46; C07K 14/00; C12N 15/113; A61K 9/51

(52) U.S. Cl. CPC A61K 38/465; C07K 14/00; A61K 9/51; C12Y 301/03067; C12N 15/1135

(57) ABSTRACT

The present invention pertains to peptide-containing complexes/nanoparticles that are useful for delivering into a cell one or more mRNA (such as therapeutic mRNA, e.g., mRNA encoding a tumor suppressor protein).

### RELATED APPLICATIONS

This application claims priority benefit to French Applications Nos. 1759645, filed Oct. 16, 2017, and U.S. Pat. No. 1,853,370, filed Apr. 17, 2018, all of which are incorporated herein by reference in their entirety for all purposes.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 737372001042SEQLIST.txt date recorded: Oct. 15, 2018, size: 37 KB).

### FIELD OF THE INVENTION

The present invention pertains to peptide-containing complexes/nanoparticles that are useful for delivering mRNA into a cell.

### BACKGROUND

The disclosures of all publications, patents, patent applications and published patent applications referred to herein are hereby incorporated herein by reference in their entirety.

In order for exogenous mRNA or RNAi to be therapeutically applicable, the mRNA or RNAi must be efficiently delivered inside of target cells, such as disease cells of a target disease. Generally, RNA delivery can be mediated by viral and non-viral vectors. Non-viral vectors can be produced at a large scale and are readily amendable to engineering. However, they suffer from low delivery efficiency and in some cases cell toxicity. On the other hand, viral vectors harness the highly evolved mechanisms that parental mRNA has developed to efficiently recognize and infect cells. However, their delivery properties can be challenging to engineer and improve. Thus, there is a need for improved methods for efficient delivery of mRNA or RNAi inside target cells.

### BRIEF SUMMARY OF THE INVENTION

The present application provides complexes and nanoparticles comprising cell-penetrating peptide that are useful for delivering into a cell one or more mRNAs (such as mRNAs encoding a therapeutic protein, e.g., tumor suppressor). Intracellular delivery of the mRNA allows for expression of a product encoded by the mRNA. In some embodiments, the mRNA encodes a protein, such as a therapeutic protein, a deficient protein, or a functional variant of a nonfunctional protein. In some embodiments, the mRNA encodes a chimeric antigen receptor (CAR). In some embodiments, the complexes and nanoparticles include an inhibitory RNA (RNAi), such as an RNAi targeting an endogenous gene. In some embodiments, the RNAi targets a disease-associated endogenous gene, e.g., an oncogene. In some embodiments, the RNAi targets an exogenous gene.

In some embodiments, there is provided an mRNA delivery complex for intracellular delivery of an mRNA comprising a cell-penetrating peptide (CPP) and the mRNA, wherein the cell-penetrating peptide is selected from the group consisting of VEPEP-3 peptides, VEPEP-6 peptides, VEPEP-9 peptides, and ADGN-100 peptides.

In some embodiments, there is provided an mRNA delivery complex for intracellular delivery of an mRNA comprising a cell-penetrating peptide (CPP) and the mRNA prepared by a process comprising the steps of: a) mixing a first solution comprising the mRNA with a second solution comprising the CPP to form a third solution, wherein the third solution comprises or is adjusted to comprise i) about 0-5% sucrose, ii) about 0-5% glucose, iii) about 0-50% DMEM, iv) about 0-80 mM NaCl, or v) about 0-20% PBS; and b) incubating the third solution to allow formation of the mRNA delivery complex. In some embodiments, the first solution comprises the mRNA in sterile water and/or the second solution comprises the CPP in sterile water. In some embodiments, the third solution is adjusted to comprise i) about 0-5% sucrose, ii) about 0-5% glucose, iii) about 0-50% DMEM, iv) about 0-80 mM NaCl, or v) about 0-20% PBS after the incubating of step b).

In some embodiments, there is provided an mRNA delivery complex for intracellular delivery of an mRNA comprising a cell-penetrating peptide (CPP) and the mRNA, wherein the mRNA encodes a therapeutic protein. In some embodiments, the therapeutic protein replaces a protein that is deficient or abnormal, augments an existing pathway, provides a novel function or activity, or interferes with a molecule or organism.

In some embodiments, there is provided an mRNA delivery complex for intracellular delivery of an mRNA comprising a cell-penetrating peptide (CPP) and the mRNA, wherein the mRNA delivery complex further comprises an RNAi. In some embodiments, the RNAi is an siRNA, a shRNA, or a miRNA. In some embodiments, the mRNA encodes a therapeutic protein for treating a disease or condition, and the RNAi targets an RNA, wherein expression of the RNA is associated with the disease or condition.

In some embodiments, according to any of the mRNA delivery complexes described above, the cell-penetrating peptide is a VEPEP-3 peptide. In some embodiments, the cell-penetrating peptide comprises an amino acid sequence selected from the group consisting of SEQ ID NOs: 1-14. In some embodiments, the cell-penetrating peptide comprises the amino acid sequence of SEQ ID NO: 75 or 76.

In some embodiments, according to any of the mRNA delivery complexes described above, the cell-penetrating peptide is a VEPEP-6 peptide. In some embodiments, the cell-penetrating peptide comprises an amino acid sequence selected from the group consisting of SEQ ID NOs: 1540. In some embodiments, the cell-penetrating peptide comprises the amino acid sequence of SEQ ID NO: 77.

In some embodiments, according to any of the mRNA delivery complexes described above, the cell-penetrating peptide is a VEPEP-9 peptide. In some embodiments, the cell-penetrating peptide comprises an amino acid sequence selected from the group consisting of SEQ ID NOs: 41-52. In some embodiments, the cell-penetrating peptide comprises the amino acid sequence of SEQ ID NO: 78.

In some embodiments, according to any of the mRNA delivery complexes described above, the cell-penetrating peptide is an ADGN-100 peptide. In some embodiments, the cell-penetrating peptide comprises an amino acid sequence selected from the group consisting of SEQ ID NOs: 53-70. In some embodiments, the cell-penetrating peptide comprises the amino acid sequence of SEQ ID NO: 79 or 80.

In some embodiments, according to any of the mRNA delivery complexes described above, the cell-penetrating peptide is covalently linked to the mRNA.

In some embodiments, according to any of the mRNA delivery complexes described above, the cell-penetrating peptide further comprises one or more moieties covalently linked to the N-terminus of the cell-penetrating peptide, wherein the one or more moieties are selected from the group consisting of an acetyl, a fatty acid, a cholesterol, a poly-ethylene glycol, a nuclear localization signal, nuclear export signal, an antibody or fragment thereof, a polysaccharide and a targeting molecule. In some embodiments, the cell-penetrating peptide comprises an acetyl group covalently linked to its N-terminus.

In some embodiments, according to any of the mRNA delivery complexes described above, the cell-penetrating peptide further comprises one or more moieties covalently linked to the C-terminus of the cell-penetrating peptide, wherein the one or more moieties are selected from the group consisting of a cysteamide, a cysteine, a thiol, an amide, a nitrilotriacetic acid optionally substituted, a carboxyl, a linear or ramified C1-C6 alkyl optionally substituted, a primary or secondary amine, an osidic derivative, a lipid, a phospholipid, a fatty acid, a cholesterol, a poly-ethylene glycol, a nuclear localization signal, nuclear export signal, an antibody or fragment thereof, a polysaccharide and a targeting molecule. In some embodiments, the cell-penetrating peptide comprises a cysteamide group covalently linked to its C-terminus.

In some embodiments, according to any of the mRNA delivery complexes described above, at least some of the cell-penetrating peptides in the mRNA delivery complex are linked to a targeting moiety by a linkage. In some embodiments, the linkage is covalent.

In some embodiments, according to any of the mRNA delivery complexes described above, the mRNA encodes a therapeutic protein. In some embodiments, the mRNA encodes a tumor suppressor protein.

In some embodiments, according to any of the mRNA delivery complexes described above, the mRNA delivery complex further comprises an RNAi. In some embodiments, the RNAi targets an oncogene for downregulation.

In some embodiments, according to any of the mRNA delivery complexes described above, the molar ratio of the cell-penetrating peptide to the mRNA is between about 1:1 and about 100:1.

In some embodiments, according to any of the mRNA delivery complexes described above, the average diameter of the mRNA delivery complex is between about 20 nm and about 1000 nm.

In some embodiments, there is provided a nanoparticle comprising a core comprising an mRNA delivery complex according to any of the embodiments described above. In some embodiments, the core further comprises one or more additional mRNA delivery complexes according to any of the embodiments, described above. In some embodiments, the core further comprises an RNAi. In some embodiments, the RNAi targets an oncogene for downregulation. In some embodiments, the RNAi is in a complex comprising a cell-penetrating peptide (CPP) and the RNAi. In some embodiments, the cell-penetrating peptide is selected from the group consisting of PEP-1 peptides, PEP-2 peptides, PEP-3 peptides, VEPEP-3 peptides, VEPEP-6 peptides, VEPEP-9 peptides, and ADGN-100 peptides.

In some embodiments, according to any of the nanoparticles described above, at least some of the cell-penetrating peptides in the nanoparticle are linked to a targeting moiety by a linkage.

In some embodiments, according to any of the nanoparticles described above, the core is coated by a shell comprising a peripheral cell-penetrating peptide. In some embodiments, the peripheral cell-penetrating peptide is selected from the group consisting of PEP-1 peptides, PEP-2 peptides, PEP-3 peptides, VEPEP-3 peptides, VEPEP-6 peptides, VEPEP-9 peptides, and ADGN-100 peptides. In some embodiments, the peripheral cell-penetrating peptide comprises an amino acid sequence selected from the group consisting of SEQ ID NOs: 1-80. In some embodiments, at least some of the peripheral cell-penetrating peptides in the shell are linked to a targeting moiety by a linkage. In some embodiments, the linkage is covalent.

In some embodiments, according to any of the nanoparticles described above, the average diameter of the nanoparticle is between about 20 nm and about 1000 nm.

In some embodiments, there is provided a pharmaceutical composition comprising an mRNA delivery complex according to any of the embodiments described above or a nanoparticle according to any of the embodiments described above, and a pharmaceutically acceptable carrier. In some embodiments, the mRNA delivery complex or nanoparticle comprises an mRNA encoding a therapeutic protein. In some embodiments, the pharmaceutical composition further comprises an inhibitory RNA (RNAi). In some embodiments, the RNAi is in the mRNA delivery complex or nanoparticle. In some embodiments, the mRNA delivery complex or nanoparticle comprises an mRNA encoding a chimeric antigen receptor (CAR).

In some embodiments, there is provided a method of preparing the mRNA delivery complex according to any of the embodiments described above, comprising combining the cell-penetrating peptide with the one or more mRNA, thereby forming the mRNA delivery complex. In some embodiments, the cell-penetrating peptide and the mRNA are combined at a molar ratio from about 1:1 to about 100:1, respectively. In some embodiments, the combining comprises mixing a first solution comprising the mRNA with a second solution comprising the CPP to form a third solution, wherein the third solution comprises or is adjusted to comprise i) about 0-5% sucrose, ii) about 0-5% glucose, iii) about 0-50% DMEM, iv) about 0-80 mM NaCl, or v) about 0-20% PBS, and wherein the third solution is incubated to allow formation of the mRNA delivery complex. In some embodiments, the first solution comprises the mRNA in sterile water and/or wherein the second solution comprises the CPP in sterile water. In some embodiments, the third solution is adjusted to comprise i) about 0-5% sucrose, ii) about 0-5% glucose, iii) about 0-50% DMEM, iv) about 0-80 mM NaCl, or v) about 0-20% PBS after incubating to form the mRNA delivery complex.

In some embodiments, there is provided a method of delivering one or more mRNA into a cell, comprising contacting the cell with an mRNA delivery complex according to any of the embodiments described above or a nanoparticle according to any of the embodiments described above, wherein the mRNA delivery complex or the nanoparticle comprises the one or more mRNA. In some embodiments, the contacting of the cell with the mRNA delivery complex or nanoparticle is carried out in vivo. In some embodiments, the contacting of the cell with the mRNA delivery complex or nanoparticle is carried out ex vivo. In some embodiments, the contacting of the cell with the mRNA delivery complex or nanoparticle is carried out in vitro. In some embodiments, the cell is a stem cell, a hematopoietic precursor cell, a granulocyte, a mast cell, a monocyte, a dendritic cell, a B cell, a T cell, a natural killer cell, a fibroblast, a muscle cell, a cardiac cell, a hepatocyte, a lung progenitor cell, or a neuronal cell. In some embodiments, the cell is a T cell. In some embodiments, the mRNA encodes a protein that is capable of modulating an immune response in an individual in which it is expressed. In some embodiments, the mRNA delivery complex or nanoparticle comprises an mRNA encoding a therapeutic protein. In some embodiments, the mRNA delivery complex or nanoparticle further comprises an inhibitory RNA (RNAi). In some embodiments, the method further comprises delivering an RNAi into the cell. In some embodiments, the mRNA delivery complex or nanoparticle comprises an mRNA encoding a chimeric antigen receptor (CAR).

In some embodiments, there is provided a method of treating a disease in an individual comprising administering to the individual an effective amount of a pharmaceutical composition according to any of the embodiments described above. In some embodiments, the pharmaceutical composition is administered via intravenous, intratumoral, intraarterial, topical, intraocular, ophthalmic, intraportal, intracranial, intracerebral, intracerebroventricular, intrathecal, intravesicular, intradermal, subcutaneous, intramuscular, intranasal, intratracheal, pulmonary, intracavity, or oral administration. In some embodiments, the pharmaceutical composition is administered via injection into a blood vessel wall or tissue surrounding the blood vessel wall. In some embodiments, the injection is through a catheter with a needle.

In some embodiments, according to any of the methods of treating a disease described above, the disease is selected from the group consisting of cancer, diabetes, autoimmune diseases, hematological diseases, cardiac diseases, vascular diseases, inflammatory diseases, fibrotic diseases, viral infectious diseases, hereditary diseases, ocular diseases, liver diseases, lung diseases, muscle diseases, protein deficiency diseases, lysosomal storage diseases, neurological diseases, kidney diseases, aging and degenerative diseases, and diseases characterized by cholesterol level abnormality.

In some embodiments, the disease is a protein deficiency disease. In some embodiments, the pharmaceutical composition comprises an mRNA delivery complex or nanoparticle comprising one or more mRNA encoding a deficient protein contributing to the disease.

In some embodiments, the disease is characterized by an abnormal protein. In some embodiments, the pharmaceutical composition comprises an mRNA delivery complex or nanoparticle comprising one or more mRNA encoding a functional variant of the non-functional protein contributing to the disease.

In some embodiments, the disease is cancer. In some embodiments, the cancer is a solid tumor, and the pharmaceutical composition comprises an mRNA delivery complex or nanoparticle comprising one or more mRNA encoding a tumor suppressor protein useful for treating the solid tumor. In some embodiments, the cancer is cancer of the liver, lung, kidney, colorectum, or pancreas. In some embodiments, the cancer is a hematological malignancy, and the pharmaceutical composition comprises an mRNA delivery complex or nanoparticle comprising one or more mRNA encoding a tumor suppressor protein useful for treating the hematological malignancy. In some embodiments, the pharmaceutical composition further comprises an RNAi that targets an oncogene involved in the cancer development and/or progression. In some embodiments, the RNAi is in the mRNA delivery complex or nanoparticle.

In some embodiments, according to any of the methods of treating a disease described above, the disease is a viral infection disease, and the pharmaceutical composition comprises an mRNA delivery complex or nanoparticle comprising one or more mRNA encoding a protein involved in the viral infectious disease development and/or progression.

In some embodiments, according to any of the methods of treating a disease described above, the disease is a hereditary disease, and the pharmaceutical composition comprises an mRNA delivery complex or nanoparticle comprising one or more mRNA encoding one or more proteins involved in the hereditary disease development and/or progression.

In some embodiments, according to any of the methods of treating a disease described above, the disease is an aging or degenerative disease, and the pharmaceutical composition comprises an mRNA delivery complex or nanoparticle comprising one or more mRNA encoding one or more proteins involved in the aging or degenerative disease development and/or progression.

In some embodiments, according to any of the methods of treating a disease described above, the disease is a fibrotic or inflammatory disease, and the pharmaceutical composition comprises an mRNA delivery complex or nanoparticle comprising one or more mRNA encoding one or more proteins involved in the fibrotic or inflammatory disease development and/or progression.

In some embodiments, according to any of the methods of treating a disease described above, the individual is human.

In some embodiments, there is provided a kit comprising a composition comprising an mRNA delivery complex according to any of the embodiments described above and/or a nanoparticle according to any of the embodiments described above.

### BRIEF DESCRIPTION OF THE FIGURES

FIGS. 1A-1F show ADGN-100/mRNA and ADGN-106/mRNA nanoparticle mean size characterization indifferent buffers. ADGN-100/mRNA particles were formed in sterile water, and then diluted with sterile water (A), 5% Sucrose (B), or 5% Glucose (C). ADGN-106/mRNA particles were formed in sterile water and then diluted in sterile water (D), 5% Sucrose (E), or 5% Glucose (F). The mean size of the ADGN/mRNA complexes was determined at 25° C. for 3 min per measurement with Zetasizer 4 apparatus (Malvern Ltd).

FIGS. 2A-2B show ADGN-100/mRNA and ADGN-106/mRNA nanoparticle's mean size characterization in different cell culture medium. ADGN-100/mRNA (A) and ADGN-106/mRNA (B) particles were formed in sterile water, then diluted in DMEM 50% or pH 7.4 (50 mM). The mean size of the ADGN/mRNA complexes was determined at 25° C. for 3 min per measurement with Zetasizer 4 apparatus (Malvern Ltd).

FIGS. 3A-3D show ADGN-100/mRNA and ADGN-106/mRNA nanoparticle's mean size characterization in different salt conditions. ADGN-100/mRNA (A,C) and ADGN-106/mRNA (B,D) particles were formed in sterile water, and then diluted in NaCl (40 mM, 80 mM, 160 mM) or in PBS (20% and 50%). The mean size of the ADGN/mRNA complexes was determined at 25° C. for 3 min per measurement with Zetasizer 4 apparatus (Malvern Ltd).

FIGS. 4A-4B show ADGN-100/mRNA and ADGN-106/mRNA nanoparticle's mean size characterization serum conditions. ADGN-100/mRNA (A) and ADGN-106/mRNA (B) particles were formed in sterile water, and then diluted in sucrose 5% in the presence or absence of 50% serum (FCS). The mean size of the ADGN/mRNA complexes was determined at 25° C. for 3 min per measurement with Zetasizer 4 apparatus (Malvern Ltd).

FIG. 5 shows luciferase expression in HepG2 cells treated with ADGN-100/mRNA and ADGN-106/mRNA nanoparticles incubated in different buffer conditions. HepG2 cells cultured in 24 well plates were transfected with ADGN-100 and ADGN-106 nanoparticles containing 0.5 μg or 1.0 μg of Luciferase mRNA. ADGN/mRNA complexes were formed in sterile water and diluted in different buffers, including sterile water, 5% Glucose, 5% Sucrose, 20% PBS (20% and 50%), Hepes pH 7.4 (50 mM), NaCl (40 mM, 80 mM, 160 mM) or DMEM (50%). Luciferase expression was monitored 30 hours post transfection and results were reported as percentage of RLU (luminecence) corresponding to untreated cells.

FIGS. 6A-6B show the evaluation of ADGN-100 and ADGN-106 for in vivo delivery of Luciferase mRNA via intravenous administration in mice. ADGN-100/Luc mRNA (A) and ADGN-106/luc mRNA (B) particles containing 10 μg mRNA were formed in sterile water, and then diluted in different buffers (sucrose 5%, glucose 5%, NaCl 80 mM or PBS 20% final concentration). Mice received IV injection of 100 μl ADGN-100/mRNA or ADGN-106/mRNA complexes. mRNA LUC expression was monitored by bioluminescence imaging at Day 3 and 6. And semi-quantitative data of luciferase signal in the liver were obtained using the manufacture's software (Living Image; PerkinElmer). Results were then expressed as values relative to day 0.

FIG. 7 shows the evaluation of ADGN-100 and ADGN-106 for in vivo delivery of Luciferase mRNA via intravenous administration in mice. ADGN-100/Luc mRNA (A) and ADGN-106/luc mRNA (B) particles containing 10 μg mRNA, were formed in sterile water then diluted in different buffers (sucrose 5%, glucose 5%, NaCl 80 mM or PBS 20% final concentration). Mice received IV injection of 100 μl ADGN-100/mRNA or ADGN-106/mRNA complexes. mRNA LUC expression was monitored by bioluminescence imaging at Day 3 and 6.

FIGS. 8A-8B show western blot analysis of PTEN expression in different cell types. The level of PTEN was evaluated in Pancreas cancer (PANC-1), Human Glioma (U25), Prostate cancer (PC3), ovarian cancer (SKOV3) and human fibroblast (HS68) cells. As shown in FIG. 8A, the level of PTEN expression was evaluated by western blots using PTEN antibody (top panel) and the PTEN protein bands were normalized with reference to β-actin (bottom panel).

FIG. 8B shows western blot analysis of PTEN expression in cancer cell type transfected with ADGN-100/mRNA and ADGN-106/mRNA complexes containing 0.5 μg and 1.0 μg PTEN mRNA. Cells were analyzed 48 hr post transfection

FIG. 9 shows the impact of ADGN mediated PTEN mRNA transfection on cancer cell proliferation. Pancreas cancer (PANC-1), Human Glioma (U25), Prostate cancer (PC3), ovarian cancer (SKOV3) and human fibroblast (HS68) cells were treated with ADGN-100/mRNA or ADGN-106/mRNA complexes containing 1 μg mRNA and cell proliferation was measured over a period of 6 days by flow cytometry assay.

FIG. 10 shows the impact of ADGN mediated PTEN mRNA transfection on cancer cell proliferation. Pancreas cancer (PANC-1). Human Glioma (U25), Prostate cancer (PC3), ovarian cancer (SKOV3) and human fibroblast (HS68) cells were treated with ADGN-100/mRNA or ADGN-106/mRNA complexes containing 0.5 μg mRNA and cell proliferation was measured over a period of 6 days by flow cytometry assay.

FIG. 11 shows the impact of ADGN mediated PTEN mRNA transfection on apoptosis rate in cancer cells. Pancreas cancer (PANC-1), Human Glioma (U25), Prostate cancer (PC3), ovarian cancer (SKOV3) and human fibroblast (HS68) cells were treated with ADGN-100/mRNA or ADGN-106/mRNA complexes (1 μg mRNA). Cell apoptosis rate (expressed as a percentage) was measured by flow cytometry using APO BrDu kit 72 hours post transfection.

FIG. 12 shows the impact of ADGN mediated PTEN mRNA transfection on cell cycle proliferation in cancer cells. Pancreas cancer (PANC-1), Human Glioma (U25), Prostate cancer (PC3), ovarian cancer (SKOV3) and human fibroblast (HS68) cells were treated with ADGN-100/mRNA or ADGN-106/mRNA complexes (1 μg mRNA). 72 hours post transfection, cell cycle stages were measured by flow cytometry using a PI (Propidium Iodide) staining kit.

FIG. 13 shows the potency of ADGN peptides (ADGN-100 and ADGN-106) to deliver PTEN mRNA in vivo in a pancreas tumor mouse model. Female nude mice 6-weeks of age were implanted in the pancreas with Human pancreatic carcinoma cell lines (Panc1-Luc). A period of 3 weeks was allowed for tumor development before the beginning of the experiments. Six groups of mice were identified Control Untreated mice (G1), mice injected with Naked mRNA 10 ug (G2), ADGN-100/5 μg PTEN mRNA dose 0.25 mg/kg (G3), ADGN-100/10 μg PTEN mRNA dose 0.5 mg/kg (G4). ADGN-106/5 μg PTEN mRNA dose 0.25 mg/kg (G5), and ADGN-106/10 μg PTEN mRNA dose 0.5 mg/kg (G6). Animal were IV tail-vein injected every 7 days. Tumor size was evaluated by bioluminescence imaging at day 0, 7, 14, 20, 26 and 33.

FIGS. 14A-14C show the potency of ADGN peptides (ADGN-100 and ADGN-106) to deliver PTEN mRNA in vivo in a pancreas tumor mouse model. A period of 3 weeks was allowed for tumor development before the beginning of the experiments. Six groups of mice were identified Control Untreated mice (G1), mice injected with Naked mRNA 10 ug (G2), ADGN-100/5 μg PTEN mRNA dose 0.25 mg/kg (G3), ADGN-100/10 μg PTEN mRNA dose 0.5 mg/kg (G4). ADGN-106/5 μg PTEN mRNA dose 0.25 mg/kg (G5), and ADGN-106/10 μg PTEN mRNA dose 0.5 mg/kg (G6). Animal were IV tail-vein injected every 7 days. Tumor size was evaluated by bioluminescence imaging at day 0, 7, 14, 20, 26 and 33. FIGS. 14A and 14B show bioluminescence imaging and a quantification of the total luminescence for the different groups at day 33. At Day 33 animals were sacrificed and tumors were harvested. FIG. 14C shows the corresponding tumors.

FIGS. 15A-15C show the potency of ADGN peptides (ADGN-100 and ADGN-106) to deliver PTEN mRNA in vivo in a pancreas tumor mouse model and impact on metastases development. A period of 6 weeks was allowed for tumor development before the beginning of the experiments. Two groups of mice were identified Control Untreated mice (G1) and mice injected with ADGN-106/10 μg PTEN mRNA dose 0.5 mg/kg (G2). Animal were IV tail-vein injected at day 0 and day 3 days. Tumor size was evaluated by bioluminescence imaging at day 0 and 7. FIG. 15A show bioluminescence imaging at day 1 and day 7 in control and treated groups. FIG. 15B show a quantification of the total luminescence for the different groups at day 0 and day 7, based on selected surface reported in FIG. 15B.

FIGS. 16A-16B show western blot analysis of KRAS level in different cell types following ADGN-106 mediated KRAS siRNA delivery. Pancreas cancer (PANC-1), Human Glioma (U25), Prostate cancer (PC3), ovarian cancer (SKOV3) and human fibroblast (HS68) cells were treated with ADGN-106/KRAS siRNA particles at 10 nM and 40 nM. FIG. 16A show a western blot analysis of the level of KRAS in the different cell types. 48 hours post transfection. The KRAS protein bands were normalized with reference to β-actin. FIG. 16B show the impact of ADGN mediated KRAS siRNA transfection on cancer cell proliferation. Pancreas cancer (PANC-1), Human Glioma (U25), Prostate cancer (PC3), ovarian cancer (SKOV3) and human fibroblast (HS68) cells were treated with ADGN-106: KRAS siRNA complexes (10 nM, 40 nM) and cell proliferation was measured 5 days post transfection by flow cytometry assay.

FIGS. 17A-17B show the impact of co administration of PTEN mRNA and KRAS siRNA in vivo using ADGN-106 on pancreas tumor mouse model. A period of 3 weeks was allowed for tumor development before the beginning of the experiments, four groups of mice were identified Control Untreated mice (G1), mice injected with ADGN-106/10 μg PTEN mRNA dose 0.5 mg/kg (G2), with ADGN-106/10 μg siRNA KRAS dose 0.5 mg/kg (G3) and ADGN-106/10 μg siRNA KRAS dose 0.5 mg/kg; ADGN-106/5 μg PTEN mRNA dose 0.25 mg/kg (G4). Animal were IV tail-vein injected every 7 days. FIG. 17A shows tumor size was evaluated by bioluminescence imaging at day 0, 7, 14, 20, and 26. FIG. 17B shows bioluminescence imaging for the different groups at day 26.

FIG. 18 shows Factor VIII level in mice treated with ADGN-100/FVIII mRNA and ADGN-106/FVIII mRNA. Transient knockdown of Factor VIII expression in the liver was obtained by IV injection of 100 μl ADGN-100/siFVIII, complex in saline buffer (90 mM NaCl) (siFVIII dose 1.0 mg/kg, 10 ug), at day 0 and day 50. Control mice, Group N1 received IV injection of 100 μl containing Naked siRNA siFVIII 10 ug and untreated group C received 100 μl of saline buffer. Then, animals were divided in 4 different groups (3 animals per group) corresponding to no treatment (G1) and treatment by injection at day 10 and 60 with FVIII mRNA/ADGN-100 (10 μg) (G2), FVIII mRNA/ADGN-106 (10 μg) (G3) and Naked FVIII mRNA (10 μg) (G4). Factor VIII level was monitored using Factor VIII Elisa kit on blood samples every 5 days.

FIG. 19 show histological analysis of the different mice group treated with ADGN/FVIII mRNA complexes. Transient knockdown of Factor VIII expression in the liver was obtained by IV injection at day 0 and day 50 of 100 μl ADGN-100/siFVII complex in saline buffer (90 mM NaCl) (siFVIII dose 1.0 mg/kg, 10 ug). Control mice (group N1) received IV injection of 100 μl containing Naked siRNA siFVIII 10 ug and mice form group C1, 100 μl of saline buffer as untreated group. Animals injected were divided in 4 different groups (3 animals per group) corresponding to no treatment (G1) and treatment by injection at day 10 and 60 with FVIII mRNA/ADGN-100 (10 μg) (G2), and FVIII mRNA/ADGN-106 (10 μg) (G3). At Day 90, animals were sacrificed and liver were harvested and analyzed by liver Histology. Thin slices of liver tissue were stained with hematoxylin and analyzed 200 light-microscopic.

FIG. 20 shows ADGN-100 mediated luciferase gene editing in PANC-1 and SKVO-3 cells expressing Luc2. PANC-1 and SKVO-3 cells cultured in 24 well plates were transfected with ADGN-100/CAS9 mRNA/gRNA Luc (0.2 μg/2 μg or 0.5 μg/5 μg). ADGN/CRISPR complexes were formed in sterile water and diluted in 5% Sucrose. As control, cells were treated with either naked CAS9 mRNA/gRNA Luc (0.5 μg/5 μg) or transfected with RNAiMAX CAS9 mRNA/gRNA Luc (0.5 μg/5 μg). Luciferase expression was monitored 48 hours post transfection and results are reported as percentage of RLU (luminecence) corresponding to untreated cells.

FIGS. 21A and 21B show the impact of co-administration of CRISPR (mRNA CAS9:Luc gRNA) in vivo using ADGN-100 in a pancreas tumor mouse model. A period of 3 weeks was allowed for tumor development before the beginning of the experiments. Mice were divided into two groups, control mice injected with saline solution and mice injected with ADGN-100/5 μg CAS9 mRNA/15 μg Luc gRNA. Animals were IV tail-vein injected on days 0, 7, and 14. FIG. 21A shows tumor size evaluated by bioluminescence imaging at day 0, 14, 20, and 28, and the corresponding tumors harvested at Day 33. FIG. 21B shows quantification of the total luminescence for the two groups at days 0, 7, 14, 20, and 28 based on the regions indicated in FIG. 21A.

FIGS. 22A and 22B show the rescue of PTEN expression and activation of apoptosis pathway in cancer cells transfected with PTEN mRNA complexed with ADGN peptides. FIG. 22A shows western blot analysis of PTEN expression in different cell types. The level of PTEN was evaluated in Pancreas cancer (PANC-1), Prostate cancer (PC3), Human glioma (U25), and ovarian cancer (SKOV3). Cells were analyzed 48 hr post transfection. FIG. 22B shows the impact of ADGN mediated PTEN mRNA transfection on apoptosis rate in cancer cells. Cell apoptosis rate (expressed as a percentage) was measured by flow cytometry using APO BrDu kit 72 hours post transfection.

FIG. 23 shows inhibition of cancer cell proliferation after ADGN-mediated PTEN mRNA transfection. Pancreas cancer (PANC-1), Human Glioma (U25), Prostate cancer (PC3), and ovarian cancer (SKOV3) cells were treated with ADGN-100/mRNA complexes containing 1 μg mRNA and cell proliferation was measured over a period of 6 days by flow cytometry assay.

FIG. 24 shows the impact of ADGN mediated PTEN mRNA transfection on cell cycle proliferation in cancer cells. Pancreas cancer (PANC-1), Human Glioma (U25), Prostate cancer (PC3), and ovarian cancer (SKOV3) cells were treated with ADGN-100/mRNA or ADGN-106/mRNA complexes (1 μg mRNA). 72 hours post transfection, cell cycle stages were measured by flow cytometry using a PI (Propidium Iodide) staining kit.

FIGS. 25A and 25B show the impact of ADGN mediated transfection with siRNA targeting KRAS G12D on proliferation in cancer cells. FIG. 25A shows western blot analysis of KRAS level in different cell types following ADGN mediated KRAS siRNA delivery, 48 hours post-transfection. Pancreas cancer (PANC-1), Human Glioma (U25), Prostate cancer (PC3), and ovarian cancer (SKOV3) cells were treated with ADGN/KRAS siRNA particles at 10 nM and 40 nM. The siRNA targets KRAS G12D. The KRAS protein bands were normalized with reference to β-actin. FIG. 25B shows cell proliferation measured over a period of 6 days by flow cytometry assay.

FIGS. 26A-26C show the impact of ADGN mediated transfection with PTEN mRNA and KRAS siRNA on tumor volume and body weight in vivo in a pancreas tumor mouse model. Female nude mice 6-weeks of age were implanted in the pancreas with Human pancreatic carcinoma cell lines (Panc1-Luc). A period of 3 weeks was allowed for tumor development before the beginning of the experiments. Six groups of mice were identified Control Untreated mice (G1), mice injected with Naked mRNA dose 0.25 mg/kg (G2), ADGN/PTEN mRNA dose 0.25 mg/kg (G3), Naked siRNA targeting KRAS dose 0.5 mg/kg (G4), ADGN/KRAS siRNA dose 0.5 mg/kg (G5), and ADGN/PTEN mRNA (0.25 mg/kg)/KRAS siRNA (0.5 mg/kg) (G6). Animal were IV tail-vein injected every 7 days. Tumor size was evaluated by bioluminescence imaging at day 0, 5, 12, 17, 22, 28.

FIGS. 27A and 27B show western blot analysis of P53 expression in different cell types. The level of p53 was evaluated in Pancreas cancer (PANC-1) Prostate cancer (PC3), ovarian cancer (SKOV3) and human fibroblast (HS68) cells. As shown in FIG. 27A, the level of P53 expression was evaluated by western blots using P53 antibody (top panel) and the P53 protein bands were normalized with reference to β-actin (bottom panel). FIG. 27B shows western blot analysis of P53 expression in cancer cell type transfected with ADGN-100/mRNA and ADGN-106/mRNA complexes containing 0.5 μg and 1.0 μg P53 mRNA. Cells were analyzed 48 hr post transfection.

FIG. 28 shows the impact of ADGN mediated P53 mRNA transfection on cancer cell proliferation. Pancreas cancer (PANC-1), Prostate cancer (PC3), ovarian cancer (SKOV3) and human fibroblast (HS68) cells were treated with ADGN-100/mRNA or ADGN-106/mRNA complexes containing 1 μg mRNA and cell proliferation was measured over a period of 6 days by flow cytometry assay.

FIG. 29 shows the impact of ADGN mediated P53 mRNA transfection on apoptosis rate in cancer cells. Pancreas cancer (PANC-1), Prostate cancer (PC3) and ovarian cancer (SKOV3) cells were treated with ADGN-100/mRNA or ADGN-106/mRNA complexes (1 μg mRNA). Cell apoptosis rate (expressed as a percentage) was measured by flow cytometry using APO BrDu kit 72 hours post transfection

FIG. 30 shows the potency of ADGN peptides (ADGN-100 and ADGN-106) to deliver P53 mRNA in vivo in a pancreas tumor mouse model. Female nude mice 6-weeks of age were implanted in the pancreas with Human pancreatic carcinoma cell lines (Panc1-Luc). A period of 3 weeks was allowed for tumor development before the beginning of the experiments. Three groups of mice were identified Control Untreated mice (G1), mice injected with Naked mRNA 10 ug (G2) and ADGN-100/10 μg P53 mRNA dose 0.5 mg/kg (G3). Animal were IV tail-vein injected every 5 days. Tumor size was evaluated by bioluminescence imaging at day 0, 7, 14 and 20.

FIGS. 31A-31B show the impact of ADGN mediated KRAS siRNA transfection on cancer cell proliferation. Pancreas cancer (PANC-1), Prostate cancer (PC3), and ovarian cancer (SKOV3) cells were treated with ADGN-106:KRAS siRNA targeting mutation at codons 12 (G12C, G12D) or 61 (Q61K) complexes at 10 nM or 40 nM. Single or mixes of SiRNA were used in complex with ADGN-106. The cell proliferation was measured 6 days post transfection by flow cytometry assay.

FIG. 32 shows the impact of ADGN mediated co delivery of P53 (tumor suppressor) or PTEN (tumor suppressor mRNA and KRAS (oncogene) siRNA on cancer cell proliferation. Pancreas cancer (PANC-1) (Panel A), and ovarian cancer (SKOV3) (Panel B) cells were treated with ADGN-100/mRNA PTEN (0.25 μg-5.7 nM), ADGN-100/mRNA P53 (0.5 μg-11.5 nM) and ADGN 106/KRAS siRNA (G12D/G12C) (5 nM) respectively. Cell proliferation was measured over a period of 8 days post-transfection

FIG. 33 shows the potency of ADGN peptides (ADGN-106) to deliver a combination of KRAS G12C/G12D siRNA in vivo in a pancreas tumor mouse model. Female nude mice 6-weeks of age were implanted in the pancreas with Human pancreatic carcinoma cell lines (Panc1-Luc). A period of 3 weeks was allowed for tumor development before the beginning of the experiments. Three groups of mice were identified Control Untreated mice (G1), mice injected with naked siRNA 10 ug (G2) and ADGN-106/10 μg G12D/G12C siRNA dose 0.5 mg/kg (G3). Animal were IV tail-vein injected every 5 days. Tumor size was evaluated by bioluminescence imaging at day 0, 7, 14, and 20.

FIG. 34 shows Factor VIII level in mice treated with ADGN-100/FVIII mRNA in IV and subcutaneously (SQ). Permanent knockdown of Factor VIII expression in the liver was obtained by IV injection of 100 μl ADGN-100/CRISPR targeting Factor VIll Exon 1, complex in saline buffer (90 mM NaCl) (dose 0.5 mg/kg, 10 ug) at day 0. Control mice from group G1 received IV injection of 100 μl of saline buffer as untreated group. After 10 days, animals injected with ADGN-100/CRISPR F VIII, were divided in 8 different groups (3 animals per group) corresponding to no treatment (G2) and treatment by SQ injection at day 10 with FVIII mRNA/ADGN-100 20 μg (G3), 40 μg (G4), 50 μg (G5), with FVIII mRNA/ADGN-106 20 μg (G6), 40 μg (G7), 50 μg (G8) and IV injection with FVIII mRNA/ADGN-100 10 μg (G9). Factor VIII level was monitored using Factor VIII Elisa kit on blood samples every 5 days.

FIG. 35 shows Factor VIII level in mice treated in SQ with multiple doses of ADGN-100/FVIII mRNA. Permanent knockdown of Factor VIII expression in the liver was obtained by IV injection of 100 μl ADGN-100/CRISPR targeting Factor VIII Exon 1, complex in saline buffer (90 mM NaCl) (dose 0.5 mg/kg, 10 ug) at day 0. Control mice from group G1 received IV injection of 100 μl of saline buffer as untreated group. After 10 days, animals injected with ADGN-100/CRISPR F VIII, were SQ injected with initial mRNA/ADGN-100 dose (40 μg single SQ injection). 2 weeks post initial administration animals were divided in 5 different groups (4 animals per group) and treated by SQ injection with different doses of mRNA/ADGN 100 complexes:FVIII mRNA/ADGN-100 10 μg (G3, Q2W), 20 μg (G4, Q3W), 30 μg (G5, Q4W), and 40 μg (G6, Q4W). FACTOR VIII levels were monitored using either Elisa Chromogenic factor VIII activity assay.

FIG. 36 shows ADGN mediated eGFP mRNA transfection on Human Osteosarcoma cell G292 cell. Human Osteosarcoma cells were treated with ADGN-100/mRNA or ADGN-106/mRNA complexes containing 0.25 μg, 0.5 μg and 1.0 μg mRNA and level of eGFP expression was measured over a period of 7 days by flow cytometry assay.

FIG. 37 shows ADGN mediated P53 mRNA transfection on Human Osteosarcoma cell G292 cell. Human Osteosarcoma cells were treated with ADGN-100/mRNA or ADGN-106/mRNA complexes containing 0.25 μg, 0.5 μg and 1.0 μg mRNA and level of P53 WT expression was quantified after 72 hr by western blot assay.

FIG. 38 shows the impact of ADGN mediated P53 mRNA transfection on Human Osteosarcoma cell G292 cell proliferation. Human Osteosarcoma cells were treated with ADGN-100/mRNA or ADGN-106/mRNA complexes containing 0.25 μg, 0.5 μg and 1.0 μg mRNA and cell proliferation was measured over a period of 7 days by MTT assay.

FIG. 39 shows the evaluation of ADGN-106 for in vivo delivery of Luciferase mRNA via nebulization administration in mice. ADGN-106/luc mRNA particles containing 10 μg mRNA were formed in sterile water/sucrose 5% buffer. Mice received non-surgical intratracheal administration of 100 μl ADGN-ADGN-106/mRNA complexes. mRNA Luc expression was monitored by bioluminescence imaging after 6 hr and 24 hr.

FIG. 40 shows the evaluation of ADGN-106 for in vivo delivery of Luciferase mRNA via nebulization administration in mice. ADGN-106/luc mRNA particles containing 10 μg mRNA were formed in sterile water/sucrose 5% buffer. Mice received non-surgical intratracheal administration of 100 μl ADGN-ADGN-106/mRNA complexes, then animal were sacrificed at 24 hrs and the different organs were analyzed for luciferase expression by bioluminescence.

FIG. 41 shows ADGN mediated eGFP mRNA transfection on Human Osteosarcoma cell G292 cell. Human Osteosarcoma cells were treated with ADGN-100/mRNA or ADGN-106/mRNA complexes containing either mRNA or 5 moU mRNA (0.5 μg and 1.0 μg). ADGN/mRNA complexes were incubated for 3 hr in the absence or in the presence of 10% or 25% SVF prior transfection. The level of eGFP expression was measured at day 6 by flow cytometry assay.

FIG. 42 shows the impact of ADGN mediated transfection with PTEN mRNA and KRAS siRNA in combination with P53 mRNA in vivo in a pancreas tumor mouse model.

FIG. 43 shows the impact of ADGN mediated transfection with PTEN mRNA and/or KRAS siRNA in combination with Abraxane on tumor volume in vivo in a pancreas tumor mouse model.

### DETAILED DESCRIPTION OF THE INVENTION

The present application provides complexes and nanoparticles comprising a cell-penetrating peptide (CPP) and one or more mRNAs, wherein the CPP is suitable for delivering into a cell the one or more mRNAs (such as mRNAs encoding a therapeutic product, e.g., a tumor suppressor). The complexes and nanoparticles may comprise a plurality of mRNAs. The mRNAs may include, for example, mRNAs encoding a therapeutic protein (e.g., tumor suppressor, immunomodulator, and the like). In some embodiments, the mRNA encodes a chimeric antigen receptor (CAR). In some embodiments, the complexes and nanoparticles preferentially localize to a target tissue, such as a disease tissue, e.g., a tumor. In some embodiments, the complexes and nanoparticles further comprise an RNAi, such as an RNAi targeting an endogenous gene. In some embodiments, the RNAi targets a disease-associated endogenous gene, e.g., an oncogene. In some embodiments, the RNAi targets an exogenous gene.

Thus, the present application in one aspect provides novel mRNA delivery complexes and nanoparticles which are described further below in more detail.

In another aspect, there are provided methods of delivering an mRNA into a cell using the cell-penetrating peptides. In another aspect, there are provided methods of delivering a complex or nanoparticle comprising an mRNA and a cell-penetrating peptide into a local tissue, organ or cell. In another aspect, there are provided methods of treating a disease or disorder by administering a complex or nanoparticle described herein comprising an mRNA and a cell-penetrating peptide to a subject.

Also provided are pharmaceutical compositions comprising a cell-penetrating peptide and one or more mRNAs (for example in the forms of complexes and nanoparticles) and uses thereof for treating diseases.

In some aspects, the mRNA delivery complexes, nanoparticles and pharmaceutical compositions have the advantage of not causing a significant toxicity while facilitate an efficiently delivery of the one or more mRNAs into an individual. For examples, in some embodiments, the administration of the mRNA delivery complexes and nanoparticles described herein do not induce a significant cytokine response (e.g., nonspecific cytokine response) and/or a significant nonspecific inflammatory response.

### Definitions

As used herein the term “wild type” is a term of the art understood by skilled persons and means the typical form of an organism, strain, gene or characteristic as it occurs in nature as distinguished from mutant or variant forms.

As used herein the term “variant” should be taken to mean the exhibition of qualities that have a pattern that deviates from what occurs in nature.

The terms “non-naturally occurring” or “engineered” are used interchangeably and indicate the involvement of the hand of man. The terms, when referring to nucleic acid molecules or polypeptides mean that the nucleic acid molecule or the polypeptide is at least substantially free from at least one other component with which they are naturally associated in nature and as found in nature.

“Complementarity” refers to the ability of a nucleic acid to form hydrogen bond(s) with another nucleic acid sequence by either traditional Watson-Crick base pairing or other non-traditional types. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50% 60%, 70%, 80%, 90%, and 100% complementary). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 60%, 65%, 70%, 75%, 80%, 85%, 90%, 95%, 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions.

As used herein, “expression” refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

The terms “therapeutic agent”, “therapeutic capable agent” or “treatment agent” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder or condition; and generally counteracting a disease, symptom, disorder or pathological condition.

As used herein, “treatment” or “treating” refers to an approach for obtaining beneficial or desired results including but not limited to a therapeutic benefit. By therapeutic benefit is meant any therapeutically relevant improvement in or effect on one or more diseases, conditions, or symptoms under treatment.

The term “effective amount” or “therapeutically effective amount” refers to the amount of an agent that is sufficient to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. The term also applies to a dose that will provide an image for detection by any one of the imaging methods described herein. The specific dose may vary depending on one or more of: the particular agent chosen, the dosing regimen to be followed, whether it is administered in combination with other compounds, timing of administration, the tissue to be imaged, and the physical delivery system in which it is carried.

As used herein, the singular form “a”. “an”, and “the” includes plural references unless indicated otherwise.

Reference to “about” a value or parameter herein includes (and describes) embodiments that are directed to that value or parameter per se. For example, description referring to “about X” includes description of “X.”

The compositions and methods of the present invention may comprise, consist of, or consist essentially of the essential elements and limitations of the invention described herein, as well as any additional or optional ingredients, components, or limitations described herein or otherwise useful.

Unless otherwise noted, technical terms are used according to conventional usage.

mRNA and RNAi

In some embodiments, an mRNA contained in an mRNA delivery complex according to any of the embodiments described herein encodes a polypeptide of interest selected from any of several target categories including, but not limited to, biologics, antibodies, vaccines, therapeutic proteins or peptides, cell penetrating peptides, secreted proteins, plasma membrane proteins, cytoplasmic or cytoskeletal proteins, intracellular membrane bound proteins, nuclear proteins, proteins associated with human disease, targeting moieties or those proteins encoded by the human genome for which no therapeutic indication has been identified but which nonetheless have utility in areas of research and discovery.

In some embodiments, an mRNA contained in an mRNA delivery complex according to any of the embodiments described herein comprises a region encoding a polypeptide of interest and a region of linked nucleosides according to any of the mRNAs described in U.S. Pat. Nos. 9,061,059 and 9,221,891, each of which is incorporated herein in its entirety.

In some embodiments, an mRNA contained in an mRNA delivery complex according to any of the embodiments described herein encodes a polypeptide variant of a reference polypeptide. In some embodiments, the polypeptide variant may have the same or a similar activity as the reference polypeptide. Alternatively, the variant may have an altered activity (e.g., increased or decreased) relative to a reference polypeptide. Generally, variants of a particular polynucleotide or polypeptide of the invention will have at least about 40%, 45%, 50%, 55%, 60%, 65%, 70%, 75%, 80%, 85%, 90%, 91%, 92%, 93%, 94%, 95%, 96%, 97%, 98%, 99% but less than 100% sequence identity to that particular reference polynucleotide or polypeptide as determined by sequence alignment programs and parameters described herein and known to those skilled in the art.

In some embodiments, an mRNA contained in an mRNA delivery complex according to any of the embodiments described herein encodes a biologic. As used herein, a “biologic” is a polypeptide-based molecule produced by the methods provided herein and which may be used to treat, cure, mitigate, prevent, or diagnose a serious or life-threatening disease or medical condition. Biologics, according to the present invention include, but are not limited to, allergenic extracts (e.g. for allergy shots and tests), blood components, gene therapy products, human tissue or cellular products used in transplantation, vaccines, monoclonal antibodies, cytokines, growth factors, enzymes, thrombolytics, and immunomodulators, among others. In some embodiments, the biologic is currently being marketed or in development.

In some embodiments, an mRNA contained in an mRNA delivery complex according to any of the embodiments described herein encodes an antibody or fragment thereof (such as an antigen-binding fragment). In some embodiments, the antibody or fragment thereof is currently being marketed or in development.

The term “antibody” includes monoclonal antibodies (including full length antibodies which have an immunoglobulin Fc region), antibody compositions with polyepitopic specificity, multispecific antibodies (e.g., bispecific antibodies, diabodies, and single-chain molecules), as well as antibody fragments. The term “immunoglobulin” (Ig) is used interchangeably with “antibody” herein. As used herein, the term “monoclonal antibody” refers to an antibody obtained from a population of substantially homogeneous antibodies, i.e., the individual antibodies comprising the population are identical except for possible naturally occurring mutations and/or post-translation modifications (e.g., isomerizations, amidations) that may be present in minor amounts. Monoclonal antibodies are highly specific, being directed against a single antigenic site.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is(are) identical with or homologous to corresponding sequences in antibodies derived from another species or belonging to another antibody class or subclass, as well as fragments of such antibodies, so long as they exhibit the desired biological activity. Chimeric antibodies of interest herein include, but are not limited to, “primatized” antibodies comprising variable domain antigen-binding sequences derived from a non-human primate (e.g., Old World Monkey. Ape etc.) and human constant region sequences.

An “antibody fragment” comprises a portion of an intact antibody, preferably the antigen binding and/or the variable region of the intact antibody. Examples of antibody fragments include Fab, Fab′, F(ab′).sub.2 and Fv fragments; diabodies; linear antibodies; nanobodies; single-chain antibody molecules and multispecific antibodies formed from antibody fragments.

Any of the five classes of immunoglobulins, IgA, IgD, IgE, IgG and IgM, may be encoded by the mRNA of the invention, including the heavy chains designated alpha, delta, epsilon, gamma and mu, respectively. Also included are polynucleotide sequences encoding the subclasses, gamma and mu. Hence any of the subclasses of antibodies may be encoded in part or in whole and include the following subclasses: IgG1, IgG2, IgG3, IgG4, IgA1 and IgA2.

In some embodiments, the antibody or fragment thereof encoded in the mRNA is utilized to treat conditions or diseases in therapeutic areas including, but not limited to, blood, cardiovascular, CNS, poisoning (including antivenoms), dermatology, endocrinology, gastrointestinal, medical imaging, musculoskeletal, oncology, immunology, respiratory, sensory and anti-infective.

In some embodiments, the antibody or fragment thereof encoded in the mRNA is a monoclonal antibody and/or a variant thereof. Variants of antibodies may also include, but are not limited to, substitutional variants, conservative amino acid substitution, insertional variants, deletional variants and/or covalent derivatives. In some embodiments, the antibody or fragment thereof encoded in the mRNA is an immunoglobulin Fc region. In some embodiments, the antibody or fragment thereof encoded in the mRNA is a variant immunoglobulin Fc region. In some embodiments, the antibody or fragment thereof encoded in the mRNA is an antibody having a variant immunoglobulin Fc region as described in U.S. Pat. No. 8,217,147 herein incorporated by reference in its entirety.

In some embodiments, an mRNA contained in an mRNA delivery complex according to any of the embodiments described herein encodes a vaccine. As used herein, a “vaccine” is a biological preparation that improves immunity to a particular disease or infectious agent. In some embodiments, the vaccine is currently being marketed or in development.

In some embodiments, the vaccine encoded by the mRNA is utilized to treat conditions or diseases in many therapeutic areas such as, but not limited to, cardiovascular, CNS, dermatology, endocrinology, oncology, immunology, respiratory, and anti-infective.

In some embodiments, an mRNA contained in an mRNA delivery complex according to any of the embodiments described herein encodes a therapeutic protein. In some embodiments, the therapeutic protein is currently being marketed or in development. In some embodiments, the therapeutic protein is useful for: (a) replacing a protein that is deficient or abnormal; (b) augmenting an existing pathway; (c) providing a novel function or activity; or (d) interfering with a molecule or organism. In some embodiments, the therapeutic protein includes, without limitation, antibody-based drugs, Fc fusion proteins, anticoagulants, blood factors, bone morphogenetic proteins, engineered protein scaffolds, enzymes, growth factors, hormones, interferons, interleukins, and thrombolytics. In some embodiments, the therapeutic protein acts by: (a) binding non-covalently to target, e.g., mAbs; (b) affecting covalent bonds, e.g., enzymes; or (c) exerting activity without specific interactions. e.g., serum albumin. In some embodiments, the therapeutic protein is a recombinant protein.

In some embodiments, the therapeutic protein encoded by the mRNA is utilized to treat conditions or diseases in many therapeutic areas such as, but not limited to, blood, cardiovascular, CNS, poisoning (including antivenoms), dermatology, endocrinology, genetic, genitourinary, gastrointestinal, musculoskeletal, oncology, and immunology, respiratory, sensory and anti-infective. In some embodiments, the therapeutic protein includes, without limitation, vascular endothelial growth factor (VEGF-A, VEGF-B, VEGF-C, VEGF-D), placenta growth factor (PGF), OX40 ligand (OX40L; CD134L), interleukin 12 (IL12), interleukin 23 (IL23), interleukin 36 γ (IL36γ), and CoA mutase.

In some embodiments, the therapeutic protein replaces a protein that is deficient or abnormal. In some embodiments, the therapeutic protein includes, without limitation, alpha 1 antitrypsin, frataxin, insulin, growth hormone (somatotropin), growth factors, hormones, dystrophin, insulin-like growth factor 1 (IGF1), factor VIII, factor IX, antithrombin 111, protein C, β-Gluco-cerebrosidase, Alglucosidase-α,α-1-iduronidase, Iduronate-2-sulphatase, Galsulphase, human α-galactosidase A, α-1-Proteinase inhibitor, lactase, pancreatic enzymes (including lipase, amylase, and protease), Adenosine deaminase, and albumin, including recombinant forms thereof.

In some embodiments, the therapeutic protein augments an existing pathway. In some embodiments, the therapeutic protein includes, without limitation, Erythropoietin, Epoetin-α, Darbepoetin-α, granulocyte colony stimulating factor (G-CSF), granulocyte-macrophage colony stimulating factor (GM-CSF), interleukin 11 (IL11), Human follicle-stimulating hormone (FSH), Human chorionic gonadotropin (HCG), Lutropin-α, Type I alpha-interferon, Interferon-α2a, Interferon-α2b, Interferon-αn3, Interferon-β1a, Interferon-β1b, Interferon-γ1b, interleukin 2 (IL2), epidermal thymocyte activating factor (ETAF), tissue plasminogen activator (tA), Urokinase, factor VIIa, activated protein C, Salmon calcitonin, human parathyroid hormone peptide (e.g., residues 1-34), incretin mimetic (e.g., exenatide), somatostatin analogue (e.g., octreotide), recombinant human bone morphogenic protein 2 (rhBMP2), Recombinant human bone morphogenic protein 7 (rhBMP7), gonadotropin releasing hormone (GnRH), keratinocyte growth factor (KGF), platelet-derived growth factor (PDGF), Trypsin, and Recombinant B-type natriuretic peptide.

In some embodiments, the therapeutic protein provides a novel function or activity. In some embodiments, the therapeutic protein includes, without limitation, Botulinum toxin type A, Botulinum toxin type B, collagenase, Human deoxy-ribonuclease 1, dornase-α, Hyaluronidase, papain, L-Asparaginase, Rasburicase, Lepirudin, Bivalirudin, Streptokinase, and anisoylated plasminogen streptokinase activator complex (APSAC).

In some embodiments, the therapeutic protein interferes with a molecule or organism. In some embodiments, the therapeutic protein includes, without limitation, anti-VEGFA antibody, anti-EGFR antibody, anti-CD52 antibody, anti-CD20 antibody, anti-HER2/Neu antibody, fusion protein between extracellular domain of human CTLA4 and the modified Fc portion of human immunoglobulin G1, interleukin 1 (IL1) receptor antagonist, anti-TNFα antibody, CD2-binding protein, anti-CD11a antibody, anti-α4-subunit of α4β1 and α4β7 integrins antibody, anti-complement protein C5 antibody, Antithymocyte globulin, Chimeric (human/mouse) IgG1, Humanized IgG1 mAb that binds the alpha chain of CD25, anti-CD3 antibody, anti-IgE antibody, Humanized IgG1 mAb that binds the A antigenic site of the F protein of respiratory syncytial virus, HIV envelope protein gp120/gp41-binding peptide, Fab fragment of chimeric (human/mouse) mAb 7E3 that binds to the glycoprotein IIb/IIIa integrin receptor, and Fab fragments of IgG that bind and neutralize venom toxins.

In some embodiments, an mRNA contained in an mRNA delivery complex according to any of the embodiments described herein encodes a fusion protein. In some embodiments, the fusion protein may be created by operably linking a charged protein to a therapeutic protein. As used herein, “operably linked” refers to the therapeutic protein and the charged protein being connected in such a way to permit the expression of the complex when introduced into the cell. As used herein, “charged protein” refers to a protein that carries a positive, negative or overall neutral electrical charge. In some embodiments, the therapeutic protein is covalently linked to the charged protein in the formation of the fusion protein. In some embodiments, the ratio of surface charge to total or surface amino acids is approximately 0.1, 0.2, 0.3, 0.4, 0.5, 0.6, 0.7, 0.8 or 0.9.

In some embodiments, an mRNA contained in an mRNA delivery complex according to any of the embodiments described herein encodes a cell penetrating peptide (CPP). In some embodiments, the CPP comprises one or more detectable labels. In some embodiments, the CPP comprises a signal sequence. As used herein, a “signal sequence” refers to a sequence of amino acid residues bound at the amino terminus of a nascent protein during protein translation. The signal sequence may be used to signal the secretion of the cell-penetrating polypeptide.

In some embodiments, the CPP encoded by the mRNA is capable of forming a complex after being translated. In some embodiments, the complex comprises a charged protein linked, e.g. covalently linked, to the cell-penetrating polypeptide.

In some embodiments, the CPP encoded by the mRNA comprises a first domain and a second domain. In some embodiments, the first domain comprises a supercharged polypeptide. In some embodiments, the second domain comprises a protein-binding partner. As used herein, “protein-binding partner” includes, but is not limited to, antibodies and functional fragments thereof, scaffold proteins, or peptides. In some embodiments, the cell-penetrating polypeptide further comprises an intracellular binding partner for the protein-binding partner. In some embodiments, the cell-penetrating polypeptide is capable of being secreted from a cell where the mRNA is introduced. In some embodiments, the cell-penetrating polypeptide is also capable of penetrating the first cell.

In some embodiments, the CPP encoded by the mRNA is capable of penetrating a second cell. In some embodiments, the second cell is from the same area as the first cell, or it may be from a different area. In some embodiments, the area includes, but is not limited to, tissues and organs. In some embodiments, the second cell is proximal or distal to the first cell.

In some embodiments, the mRNA encodes a cell-penetrating polypeptide comprising a protein-binding partner. In some embodiments, the protein binding partner includes, but is not limited to, an antibody, a supercharged antibody or a functional fragment. In some embodiments, the mRNA is introduced into the cell where a cell-penetrating polypeptide comprising the protein-binding partner is introduced.

In some embodiments, an mRNA contained in an mRNA delivery complex according to any of the embodiments described herein encodes a secreted protein. The secreted proteins may be selected from those described herein or those in US Patent Publication, 20100255574, the contents of which are incorporated herein by reference in their entirety.

In one embodiment, these may be used in the manufacture of large quantities of valuable human gene products.

In some embodiments, an mRNA contained in an mRNA delivery complex according to any of the embodiments described herein encodes a protein of the plasma membrane.

In some embodiments, an mRNA contained in an mRNA delivery complex according to any of the embodiments described herein encodes a cytoplasmic or cytoskeletal protein.

In some embodiments, an mRNA contained in an mRNA delivery complex according to any of the embodiments described herein encodes an intracellular membrane bound protein.

In some embodiments, an mRNA contained in an mRNA delivery complex according to any of the embodiments described herein encodes a nuclear protein.

In some embodiments, an mRNA contained in an mRNA delivery complex according to any of the embodiments described herein encodes a protein associated with human disease.

In some embodiments, an mRNA contained in an mRNA delivery complex according to any of the embodiments described herein encodes a protein with a presently unknown therapeutic function.

In some embodiments, an mRNA contained in an mRNA delivery complex according to any of the embodiments described herein encodes a targeting moiety. These include a protein-binding partner or a receptor on the surface of the cell, which functions to target the cell to a specific tissue space or to interact with a specific moiety, either in vivo or in vitro. Suitable protein-binding partners include, but are not limited to, antibodies and functional fragments thereof, scaffold proteins, or peptides. Additionally, mRNA can be employed to direct the synthesis and extracellular localization of lipids, carbohydrates, or other biological moieties or biomolecules.

In some embodiments, the mRNAs may be used to produce polypeptide libraries. These libraries may arise from the production of a population of mRNA, each containing various structural or chemical modification designs. In this embodiment, a population of mRNA may comprise a plurality of encoded polypeptides, including but not limited to, an antibody or antibody fragment, protein binding partner, scaffold protein, and other polypeptides taught herein or known in the art. In a preferred embodiment, the mRNA may be suitable for direct introduction into a target cell or culture which in turn may synthesize the encoded polypeptides.

In certain embodiments, multiple variants of a protein, each with different amino acid modification(s), may be produced and tested to determine the best variant in terms of pharmacokinetics, stability, biocompatibility, and/or biological activity, or a biophysical property such as expression level. Such a library may contain 10, 10.sup.2, 10.sup.3, 10.sup.4, 10.sup.5, 10.sup.6, 10.sup.7, 10.sup.8, 10.sup.9, or over 10.sup.9 possible variants (including, but not limited to, substitutions, deletions of one or more residues, and insertion of one or more residues).

In some embodiments, an mRNA contained in an mRNA delivery complex according to any of the embodiments described herein encodes an antimicrobial peptides (AMP) or antiviral peptides (AVP). AMPs and AVPs have been isolated and described from a wide range of animals such as, but not limited to, microorganisms, invertebrates, plants, amphibians, birds, fish, and mammals (Wang et al., Nucleic Acids Res. 2009; 37 (Database issue):D933-7). For example, anti-microbial polypeptides are described in Antimicrobial Peptide Database (aps.unmc.edu/AP/main.php; Wang et al., Nucleic Acids Res. 2009: 37 (Database issue):D933-7), CAMP: Collection of Anti-Microbial Peptides (www.bicnirrh.res.in/antimicrobial/); Thomas et al., Nucleic Acids Res. 2010; 38 (Database issue):D774-80), U.S. Pat. Nos. 5,221,732, 5,447,914, 5,519,115, 5,607,914, 5,714,577, 5,734,015. U.S. Pat. No. 5,798,336. U.S. Pat. No. 5,821,224. U.S. Pat. Nos. 5,849,490, 5,856,127, 5,905,187, 5,994,308, 5,998,374, 6,107,460, 6,191,254, 6,211,148, 6,300,489, 6,329,504, 6,399,370, 6,476,189, 6,478,825, 6,492,328, 6,514,701, 6,573,361, 6,573,361, 6,576,755, 6,605,698, 6,624,140, 6,638,531, 6,642,203, 6,653,280, 6,696,238, 6,727,066, 6,730,659, 6,743,598, 6,743,769, 6,747,007, 6,790,833, 6,794,490, 6,818,407, 6,835,536, 6,835,713, 6,838,435, 6,872,705, 6,875,907, 6,884,776, 6,887,847, 6,906,035, 6,911,524, 6,936,432, 7,001,924, 7,071,293, 7,078,380, 7,091,185, 7,094,759, 7,166,769, 7,244,710, 7,314,858, and 7,582,301, the contents of which are incorporated by reference in their entirety.

The anti-microbial polypeptides described herein may block cell fusion and/or viral entry by one or more enveloped viruses (e.g., HIV, HCV). For example, the anti-microbial polypeptide can comprise or consist of a synthetic peptide corresponding to a region, e.g., a consecutive sequence of at least about 5, 10, 15, 20, 25, 30, 35, 40, 45, 50, 55, or 60 amino acids of the transmembrane subunit of a viral envelope protein, e.g., HIV-1 gp120 or gp41. The amino acid and nucleotide sequences of HIV-1 gp120 or gp41 are described in, e.g., Kuiken et al., (2008). “HIV Sequence Compendium,” Los Alamos National Laboratory.

In some embodiments, the anti-microbial polypeptide may have at least about 75%, 80%, 85%, 90%, 95%, 100% sequence homology to the corresponding viral protein sequence. In some embodiments, the anti-microbial polypeptide may have at least about 75%, 80%, 85%, 90%, 95%, or 100% sequence homology to the corresponding viral protein sequence.

In other embodiments, the anti-microbial polypeptide may comprise or consist of a synthetic peptide corresponding to a region, e.g., a consecutive sequence of at least about 5, 10, 15, 20, 25, 30, 35, 40, 45, 50, 55, or 60 amino acids of the binding domain of a capsid binding protein. In some embodiments, the anti-microbial polypeptide may have at least about 75%, 80%, 85%, 90%, 95%, or 100% sequence homology to the corresponding sequence of the capsid binding protein.

The anti-microbial polypeptides described herein may block protease dimerization and inhibit cleavage of viral proproteins (e.g., HIV Gag-pol processing) into functional proteins thereby preventing release of one or more enveloped viruses (e.g., HIV, HCV). In some embodiments, the anti-microbial polypeptide may have at least about 75%, 80%, 85%, 90%, 95%, 100% sequence homology to the corresponding viral protein sequence.

In other embodiments, the anti-microbial polypeptide can comprise or consist of a synthetic peptide corresponding to a region, e.g., a consecutive sequence of at least about 5, 10, 15, 20, 25, 30, 35, 40, 45, 50, 55, or 60 amino acids of the binding domain of a protease binding protein. In some embodiments, the anti-microbial polypeptide may have at least about 75%, 80%, 85%, 90%, 95%, 100% sequence homology to the corresponding sequence of the protease binding protein.

The anti-microbial polypeptides described herein can include an in vitro-evolved polypeptide directed against a viral pathogen.

Anti-microbial polypeptides (AMPs) are small peptides of variable length, sequence and structure with broad spectrum activity against a wide range of microorganisms including, but not limited to, bacteria, viruses, fungi, protozoa, parasites, prions, and tumor/cancer cells. (See, e.g., Zaiou, J Mol Med, 2007; 85:317; herein incorporated by reference in its entirety). It has been shown that AMPs have broad-spectrum of rapid onset of killing activities, with potentially low levels of induced resistance and concomitant broad anti-inflammatory effects.

In some embodiments, the anti-microbial polypeptide (e.g., an anti-bacterial polypeptide) may be under 10 kDa, e.g., under 8 kDa, 6 kDa, 4 kDa, 2 kDa, or kDa. In some embodiments, the anti-microbial polypeptide (e.g., an anti-bacterial polypeptide) consists of from about 6 to about 100 amino acids, e.g., from about 6 to about 75 amino acids, about 6 to about 50 amino acids, about 6 to about 25 amino acids, about 25 to about 100 amino acids, about 50 to about 100 amino acids, or about 75 to about 100 amino acids. In certain embodiments, the anti-microbial polypeptide (e.g., an anti-bacterial polypeptide) may consist of from about 15 to about 45 amino acids. In some embodiments, the anti-microbial polypeptide (e.g., an anti-bacterial polypeptide) is substantially cationic.

In some embodiments, the anti-microbial polypeptide (e.g., an anti-bacterial polypeptide) may be substantially amphipathic. In certain embodiments, the anti-microbial polypeptide (e.g., an anti-bacterial polypeptide) may be substantially cationic and amphipathic. In some embodiments, the anti-microbial polypeptide (e.g., an anti-bacterial polypeptide) may be cytostatic to a Gram-positive bacterium. In some embodiments, the anti-microbial polypeptide (e.g., an anti-bacterial polypeptide) may be cytotoxic to a Gram-positive bacterium. In some embodiments, the anti-microbial polypeptide (e.g., an anti-bacterial polypeptide) may be cytostatic and cytotoxic to a Gram-positive bacterium. In some embodiments, the anti-microbial polypeptide (e.g., an anti-bacterial polypeptide) may be cytostatic to a Gram-negative bacterium. In some embodiments, the anti-microbial polypeptide (e.g., an anti-bacterial polypeptide) may be cytotoxic to a Gram-negative bacterium. In some embodiments, the anti-microbial polypeptide (e.g., an anti-bacterial polypeptide) may be cytostatic and cytotoxic to a Gram-positive bacterium. In some embodiments, the anti-microbial polypeptide may be cytostatic to a virus, fungus, protozoan, parasite, prion, or a combination thereof. In some embodiments, the anti-microbial polypeptide may be cytotoxic to a virus, fungus, protozoan, parasite, prion, or a combination thereof. In certain embodiments, the anti-microbial polypeptide may be cytostatic and cytotoxic to a virus, fungus, protozoan, parasite, prion, or a combination thereof. In some embodiments, the anti-microbial polypeptide may be cytotoxic to a tumor or cancer cell (e.g., a human tumor and/or cancer cell). In some embodiments, the anti-microbial polypeptide may be cytostatic to a tumor or cancer cell (e.g., a human tumor and/or cancer cell). In certain embodiments, the anti-microbial polypeptide may be cytotoxic and cytostatic to a tumor or cancer cell (e.g., a human tumor or cancer cell). In some embodiments, the anti-microbial polypeptide (e.g., an anti-bacterial polypeptide) may be a secreted polypeptide.

In some embodiments, the anti-microbial polypeptide comprises or consists of a defensin. Exemplary defensins include, but are not limited to, .alpha.-defensins (e.g., neutrophil defensin 1, defensin alpha 1, neutrophil defensin 3, neutrophil defensin 4, defensin 5, defensin 6), .beta.-defensins (e.g., beta-defensin 1, beta-defensin 2, beta-defensin 103, beta-defensin 107, beta-defensin 110, beta-defensin 136), and .theta.-defensins. In other embodiments, the anti-microbial polypeptide comprises or consists of a cathelicidin (e.g., hCAP18).

Anti-viral polypeptides (AVPs) are small peptides of variable length, sequence and structure with broad spectrum activity against a wide range of viruses. See, e.g., Zaiou, J Mol Med, 2007; 85:317. It has been shown that AVPs have a broad-spectrum of rapid onset of killing activities, with potentially low levels of induced resistance and concomitant broad anti-inflammatory effects. In some embodiments, the anti-viral polypeptide is under 10 kDa, e.g., under 8 kDa, 6 kDa, 4 kDa, 2 kDa, or 1 kDa. In some embodiments, the anti-viral polypeptide comprises or consists of from about 6 to about 100 amino acids, e.g., from about 6 to about 75 amino acids, about 6 to about 50 amino acids, about 6 to about 25 amino acids, about 25 to about 100 amino acids, about 50 to about 100 amino acids, or about 75 to about 100 amino acids. In certain embodiments, the anti-viral polypeptide comprises or consists of from about 15 to about 45 amino acids. In some embodiments, the anti-viral polypeptide is substantially cationic. In some embodiments, the anti-viral polypeptide is substantially amphipathic. In certain embodiments, the anti-viral polypeptide is substantially cationic and amphipathic. In some embodiments, the anti-viral polypeptide is cytostatic to a virus. In some embodiments, the anti-viral polypeptide is cytotoxic to a virus. In some embodiments, the anti-viral polypeptide is cytostatic and cytotoxic to a virus. In some embodiments, the anti-viral polypeptide is cytostatic to a bacterium, fungus, protozoan, parasite, prion, or a combination thereof. In some embodiments, the anti-viral polypeptide is cytotoxic to a bacterium, fungus, protozoan, parasite, prion or a combination thereof. In certain embodiments, the anti-viral polypeptide is cytostatic and cytotoxic to a bacterium, fungus, protozoan, parasite, prion, or a combination thereof. In some embodiments, the anti-viral polypeptide is cytotoxic to a tumor or cancer cell (e.g., a human cancer cell). In some embodiments, the anti-viral polypeptide is cytostatic to a tumor or cancer cell (e.g., a human cancer cell). In certain embodiments, the anti-viral polypeptide is cytotoxic and cytostatic to a tumor or cancer cell (e.g., a human cancer cell). In some embodiments, the anti-viral polypeptide is a secreted polypeptide.

In some embodiments, the mRNA incorporates one or more cytotoxic nucleosides. For example, cytotoxic nucleosides may be incorporated into mRNA such as bifunctional modified RNAs or mRNAs. Cytotoxic nucleoside anticancer agents include, but are not limited to, adenosine arabinoside, cytarabine, cytosine arabinoside, 5-fluorouracil, fludarabine, floxuridine. FTORAFUR® (a combination of tegafur and uracil), tegafur ((RS)-5-fluoro-1-(tetrahydrofuran-2-yl)pyrimidine-2,4(1H,3H)-dione), and 6-mercaptopurine.

A number of cytotoxic nucleoside analogues are in clinical use, or have been the subject of clinical trials, as anticancer agents. Examples of such analogues include, but are not limited to, cytarabine, gemcitabine, troxacitabine, decitabine, tezacitabine, 2′-deoxy-2′-methylidenecytidine (DMDC), cladribine, clofarabine, 5-azacytidine, 4′-thio-aracytidine, cyclopentenylcytosine and 1-(2-C-cyano-2-deoxy-beta-D-arabino-pentofuranosyl)-cytosine. Another example of such a compound is fludarabine phosphate. These compounds may be administered systemically and may have side effects which are typical of cytotoxic agents such as, but not limited to, little or no specificity for tumor cells over proliferating normal cells.

A number of prodrugs of cytotoxic nucleoside analogues are also reported in the art. Examples include, but are not limited to, N4-behenoyl-1-beta-D-arabinofuranosylcytosine, N4-octadecyl-1-beta-D-arabinofuranosylcytosine, N4-palmitoyl-1-(2-C-cyano-2-deoxy-beta-D-arabino-pentofuranosyl) cytosine, and P4055 (cytarabine 5′-elaidic acid ester). In general, these prodrugs may be converted into the active drugs mainly in the liver and systemic circulation and display little or no selective release of active drug in the tumor tissue. For example, capecitabine, a prodrug of 5′-deoxy-5-fluorocytidine (and eventually of 5-fluorouracil), is metabolized both in the liver and in the tumor tissue. A series of capecitabine analogues containing “an easily hydrolysable radical under physiological conditions” has been claimed by Fujiu et al. (U.S. Pat. No. 4,966,891) and is herein incorporated by reference. The series described by Fujiu includes N4 alkyl and aralkyl carbamates of 5′-deoxy-5-fluorocytidine and the implication that these compounds will be activated by hydrolysis under normal physiological conditions to provide 5′-deoxy-5-fluorocytidine.

A series of cytarabine N4-carbamates has been by reported by Fadl et al (Pharmazie. 1995, 50, 382-7, herein incorporated by reference) in which compounds were designed to convert into cytarabine in the liver and plasma. WO 2004/041203, herein incorporated by reference, discloses prodrugs of gemcitabine, where some of the prodrugs are N4-carbamates. These compounds were designed to overcome the gastrointestinal toxicity of gemcitabine and were intended to provide gemcitabine by hydrolytic release in the liver and plasma after absorption of the intact prodrug from the gastrointestinal tract. Nomura et al (Bioorg Med. Chem. 2003, 11, 2453-61, herein incorporated by reference) have described acetal derivatives of 1-(3-C-ethynyl-.beta.-D-ribo-pentofaranosyl) cytosine which, on bioreduction, produced an intermediate that required further hydrolysis under acidic conditions to produce a cytotoxic nucleoside compound.

Cytotoxic nucleotides which may be chemotherapeutic also include, but are not limited to, pyrazolo[3,4-D]-pyrimidines, allopurinol, azathioprine, capecitabine, cytosine arabinoside, fluorouracil, mercaptopurine, 6-thioguanine, acyclovir, ara-adenosine, ribavirin, 7-deaza-adenosine, 7-deaza-guanosine, 6-aza-uracil, 6-aza-cytidine, thymidine ribonucleotide, 5-bromodeoxyuridine, 2-chloro-purine, and inosine, or combinations thereof.

Untranslated regions (UTRs) of a gene are transcribed but not translated. The 5′UTR starts at the transcription start site and continues to the start codon but does not include the start codon: whereas, the 3′UTR starts immediately following the stop codon and continues until the transcriptional termination signal. There is growing body of evidence about the regulatory roles played by the UTRs in terms of stability of the nucleic acid molecule and translation. The regulatory features of a UTR can be incorporated into the mRNA of the present invention to enhance the stability of the molecule. The specific features can also be incorporated to ensure controlled down-regulation of the transcript in case they are misdirected to undesired organs sites.

Natural 5′UTRs bear features which play roles in for translation initiation. They harbor signatures like Kozak sequences which are commonly known to be involved in the process by which the ribosome initiates translation of many genes. Kozak sequences have the consensus CCRCCAUGG (SEQ ID NO: 91), where R is a purine (adenine or guanine) three bases upstream of the start codon (AUG), which is followed by another ‘G’. 5′UTR also have been known to form secondary structures which are involved in elongation factor binding.

By engineering the features typically found in abundantly expressed genes of specific target organs, one can enhance the stability and protein production of the mRNA of the invention. For example, introduction of 5′ UTR of liver-expressed mRNA, such as albumin, serum amyloid A, Apolipoprotein A/B/E, transferrin, alpha fetoprotein, erythropoietin, or Factor VIII, could be used to enhance expression of a nucleic acid molecule, such as a mRNA, in hepatic cell lines or liver. Likewise, use of 5′ UTR from other tissue-specific mRNA to improve expression in that tissue is possible for muscle (MyoD, Myosin, Myoglobin, Myogenin, Herculin), for endothelial cells (Tie-1, CD36), for myeloid cells (C/EBP, AML1, G-CSF, GM-CSF, CD1l b, MSR, Fr-1, i-NOS), for leukocytes (CD45, CD18), for adipose tissue (CD36, GLUT4, ACRP30, adiponectin) and for lung epithelial cells (SP-A/B/C/D).

Other non-UTR sequences may be incorporated into the 5′ (or 3′ UTR) UTRs. For example, introns or portions of introns sequences may be incorporated into the flanking regions of the mRNA of the invention. Incorporation of intronic sequences may increase protein production as well as mRNA levels.

UTRs are known to have stretches of Adenosines and Uridines embedded in them. These AU rich signatures are particularly prevalent in genes with high rates of turnover. Based on their sequence features and functional properties, the AU rich elements (AREs) can be separated into three classes (Chen et al, 1995): Class I AREs contain several dispersed copies of an AUUUA motif within U-rich regions. C-Myc and MyoD contain class I AREs. Class II AREs possess two or more overlapping UUAUUUA(U/A)(U/A) nonamers. Molecules containing this type of AREs include GM-CSF and TNF-a. Class III ARES are less well defined. These U rich regions do not contain an AUUUA motif, c-Jun and Myogenin are two well-studied examples of this class. Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into the 3′ UTR of nucleic acid molecules will lead to HuR binding and thus, stabilization of the message in vivo.

Introduction, removal or modification of 3′ UTR AU rich elements (AREs) can be used to modulate the stability of mRNA of the invention. When engineering specific mRNA, one or more copies of an ARE can be introduced to make mRNA of the invention less stable and thereby curtail translation and decrease production of the resultant protein. Likewise, AREs can be identified and removed or mutated to increase the intracellular stability and thus increase translation and production of the resultant protein. Transfection experiments can be conducted in relevant cell lines, using mRNA of the invention and protein production can be assayed at various time points post-transfection. For example, cells can be transfected with different ARE-engineering molecules and by using an ELISA kit to the relevant protein and assaying protein produced at 6 hour, 12 hour, 24 hour, 48 hour, and 7 days post-transfection.

MicroRNAs (or miRNA) are 19-25 nucleotide long noncoding RNAs that bind to the 3′UTR of nucleic acid molecules and down-regulate gene expression either by reducing nucleic acid molecule stability or by inhibiting translation. In some embodiments, an mRNA contained in an mRNA delivery complex according to any of the embodiments described herein comprises one or more microRNA target sequences, microRNA sequences, or microRNA seeds. Such sequences may correspond to any known microRNA such as those taught in US Publication US2005/0261218 and US Publication US2005/0059005, the contents of which are incorporated herein by reference in their entirety.

A microRNA sequence comprises a “seed” region, i.e., a sequence in the region of positions 2-8 of the mature microRNA, which sequence has perfect Watson-Crick complementarity to the miRNA target sequence. A microRNA seed may comprise positions 2-8 or 2-7 of the mature microRNA. In some embodiments, a microRNA seed may comprise 7 nucleotides (e.g., nucleotides 2-8 of the mature microRNA), wherein the seed-complementary site in the corresponding miRNA target is flanked by an adenine (A) opposed to microRNA position 1. In some embodiments, a microRNA seed may comprise 6 nucleotides (e.g., nucleotides 2-7 of the mature microRNA), wherein the seed-complementary site in the corresponding miRNA target is flanked by an adenine (A) opposed to microRNA position 1. See for example, Grimson A, Farh K K, Johnston W K, Garrett-Engele P, Lim L P, Bartel D P; Mol. Cell. 2007 Jul. 6: 27(1):91-105; each of which is herein incorporated by reference in their entirety. The bases of the microRNA seed have complete complementarity with the target sequence. By engineering microRNA target sequences into the 3′UTR of mRNA of the invention one can target the molecule for degradation or reduced translation, provided the microRNA in question is available. This process will reduce the hazard of off target effects upon nucleic acid molecule delivery. Identification of microRNA, microRNA target regions, and their expression patterns and role in biology have been reported (Bonauer et al., Curr Drug Targets 2010 11:943-949: Anand and Cheresh Curr Opin Hematol 2011 18:171-176; Contreras and Rao Leukemia 2012 26:404-413 (2011 Dec. 20. doi: 10.1038/leu.2011.356); Bartel Cell 2009 136:215-233; Landgraf et al, Cell, 2007 129:1401-1414; each of which is herein incorporated by reference in its entirety).

For example, if the nucleic acid molecule is an mRNA and is not intended to be delivered to the liver but ends up there, then miR-122, a microRNA abundant in liver, can inhibit the expression of the gene of interest if one or multiple target sites of miR-122 are engineered into the 3′ UTR of the mRNA. Introduction of one or multiple binding sites for different microRNA can be engineered to further decrease the longevity, stability, and protein translation of a mRNA.

As used herein, the term “microRNA site” refers to a microRNA target site or a microRNA recognition site, or any nucleotide sequence to which a microRNA binds or associates. It should be understood that “binding” may follow traditional Watson-Crick hybridization rules or may reflect any stable association of the microRNA with the target sequence at or adjacent to the microRNA site.

Conversely, for the purposes of the mRNA of the present invention, microRNA binding sites can be engineered out of (i.e. removed from) sequences in which they naturally occur in order to increase protein expression in specific tissues. For example, miR-122 binding sites may be removed to improve protein expression in the liver. Regulation of expression in multiple tissues can be accomplished through introduction or removal or one or several microRNA binding sites.

Examples of tissues where microRNA are known to regulate mRNA, and thereby protein expression, include, but are not limited to, liver (miR-122), muscle (miR-133, miR-206, miR-208), endothelial cells (miR-17-92, miR-126), myeloid cells (miR-142-3p, miR-142-5p, miR-16, miR-21, miR-223, miR-24, miR-27), adipose tissue (let-7, miR-30c), heart (miR-1d, miR-149), kidney (miR-192, miR-194, miR-204), and lung epithelial cells (let-7, miR-133, miR-126). MicroRNA can also regulate complex biological processes such as angiogenesis (miR-132) (Anand and Cheresh Curr Opin Hematol 2011 18:171-176: herein incorporated by reference in its entirety). In the mRNA of the present invention, binding sites for microRNAs that are involved in such processes may be removed or introduced, in order to tailor the expression of the mRNA expression to biologically relevant cell types or to the context of relevant biological processes. A listing of MicroRNA, miR sequences and miR binding sites is listed in Table 9 of U.S. Provisional Application No. 61/753,661 filed Jan. 17, 2013, in Table 9 of U.S. Provisional Application No. 61/754,159 filed Jan. 18, 2013, and in Table 7 of U.S. Provisional Application No. 61/758,921 filed Jan. 31, 2013, each of which are herein incorporated by reference in their entireties.

Examples of use of microRNA to drive tissue or disease-specific gene expression are listed (Getner and Naldini, Tissue Antigens. 2012, 80:393-403; herein incorporated by reference in its entirety). In addition, microRNA seed sites can be incorporated into mRNA to decrease expression in certain cells which results in a biological improvement. An example of this is incorporation of miR-142 sites into a UGT1A1-expressing lentiviral vector. The presence of miR-142 seed sites reduced expression in hematopoietic cells, and as a consequence reduced expression in antigen-presentating cells, leading to the absence of an immune response against the virally expressed UGT1A1 (Schmitt et al., Gastroenterology 2010; 139:999-1007; Gonzalez-Asequinolaza et al. Gastroenterology 2010, 139:726-729; both herein incorporated by reference in its entirety). Incorporation of miR-142 sites into modified mRNA could not only reduce expression of the encoded protein in hematopoietic cells, but could also reduce or abolish immune responses to the mRNA-encoded protein. Incorporation of miR-142 seed sites (one or multiple) into mRNA would be important in the case of treatment of patients with complete protein deficiencies (UGT1A1 type I, LDLR-deficient patients, CRIM-negative Pompe patients, etc.).

Lastly, through an understanding of the expression patterns of microRNA in different cell types, an mRNA contained in an mRNA delivery complex according to any of the embodiments described herein can be engineered for more targeted expression in specific cell types or only under specific biological conditions. Through introduction of tissue-specific microRNA binding sites, mRNA could be designed that would be optimal for protein expression in a tissue or in the context of a biological condition.

Transfection experiments can be conducted in relevant cell lines, using an mRNA contained in an mRNA delivery complex according to any of the embodiments described herein and protein production can be assayed at various time points post-transfection. For example, cells can be transfected with different microRNA binding site-engineering mRNAs and by using an ELISA kit to the relevant protein and assaying protein produced at 6 hour, 12 hour, 24 hour, 48 hour, 72 hour and 7 days post-transfection. In vivo experiments can also be conducted using microRNA-binding site-engineered molecules to examine changes in tissue-specific expression of formulated mRNA.

The 5′ cap structure of an mRNA is involved in nuclear export, increasing mRNA stability and binds the mRNA Cap Binding Protein (CBP), which is responsible for mRNA stability in the cell and translation competency through the association of CBP with poly(A) binding protein to form the mature cyclic mRNA species. The cap further assists the removal of 5′ proximal introns removal during mRNA splicing.

Endogenous mRNA molecules may be 5′-end capped generating a 5′-ppp-5′-triphosphate linkage between a terminal guanosine cap residue and the 5′-terminal transcribed sense nucleotide of the mRNA molecule. This 5′-guanylate cap may then be methylated to generate an N7-methyl-guanylate residue. The ribose sugars of the terminal and/or anteterminal transcribed nucleotides of the 5′ end of the mRNA may optionally also be 2′-O-methylated. 5′-decapping through hydrolysis and cleavage of the guanylate cap structure may target a nucleic acid molecule, such as an mRNA molecule, for degradation.

Modifications to an mRNA contained in an mRNA delivery complex according to any of the embodiments described herein may generate a non-hydrolyzable cap structure preventing decapping and thus increasing mRNA half-life. Because cap structure hydrolysis requires cleavage of 5′-ppp-5′ phosphorodiester linkages, modified nucleotides may be used during the capping reaction. For example, a Vaccinia Capping Enzyme from New England Biolabs (Ipswich, Mass.) may be used with .alpha.-thio-guanosine nucleotides according to the manufacturer's instructions to create a phosphorothioate linkage in the 5′-ppp-5′ cap. Additional modified guanosine nucleotides may be used such as .alpha.-methyl-phosphonate and seleno-phosphate nucleotides.

Additional modifications include, but are not limited to, 2′-O-methylation of the ribose sugars of 5′-terminal and/or 5′-anteterminal nucleotides of the mRNA (as mentioned above) on the 2′-hydroxyl group of the sugar ring. Multiple distinct 5′-cap structures can be used to generate the 5′-cap of a nucleic acid molecule, such as an mRNA molecule.

Cap analogs, which herein are also referred to as synthetic cap analogs, chemical caps, chemical cap analogs, or structural or functional cap analogs, differ from natural (i.e. endogenous, wild-type or physiological) 5′-caps in their chemical structure, while retaining cap function. Cap analogs may be chemically (i.e. non-enzymatically) or enzymatically synthesized and/or linked to a nucleic acid molecule.

For example, the Anti-Reverse Cap Analog (ARCA) cap contains two guanines linked by a 5′-5′-triphosphate group, wherein one guanine contains an N7 methyl group as well as a 3′-O-methyl group (i.e., N7,3′-O-dimethyl-guanosine-5′-triphosphate-5′-guanosine (m.sup.7G-3′mppp-G; which may equivalently be designated 3′ O-Me-m7G(5′)ppp(5′)G). The 3′-O atom of the other, unmodified, guanine becomes linked to the 5′-terminal nucleotide of the capped nucleic acid molecule (e.g. an mRNA). The N7- and 3′-O-methylated guanine provides the terminal moiety of the capped nucleic acid molecule (e.g. mRNA).

Another exemplary cap is mCAP, which is similar to ARCA but has a 2′-.beta.-methyl group on guanosine (i.e. N7,2′-dimethyl-guanosine-5′-triphosphate-5′-guanosine, m.sup.7Gm-ppp-G).

While cap analogs allow for the concomitant capping of a nucleic acid molecule in an in vitro transcription reaction, up to 20% of transcripts can remain uncapped. This, as well as the structural differences of a cap analog from an endogenous 5′-cap structures of nucleic acids produced by the endogenous, cellular transcription machinery, may lead to reduced translational competency and reduced cellular stability.

An mRNA contained in an mRNA delivery complex according to any of the embodiments described herein may also be capped post-transcriptionally, using enzymes, in order to generate more authentic 5′-cap structures. As used herein, the phrase “more authentic” refers to a feature that closely mirrors or mimics, either structurally or functionally, an endogenous or wild type feature. That is, a “more authentic” feature is better representative of an endogenous, wild-type, natural or physiological cellular function and/or structure as compared to synthetic features or analogs, etc., of the prior art, or which outperforms the corresponding endogenous, wild-type, natural or physiological feature in one or more respects. Non-limiting examples of more authentic 5′ cap structures of the present invention are those which, among other things, have enhanced binding of cap binding proteins, increased half-life, reduced susceptibility to 5′ endonucleases and/or reduced 5′decapping, as compared to synthetic 5′cap structures known in the art (or to a wild-type, natural or physiological 5′cap structure). For example, recombinant Vaccinia Virus Capping Enzyme and recombinant 2′-O-methyltransferase enzyme can create a canonical 5′-5′-triphosphate linkage between the 5′-terminal nucleotide of an mRNA and a guanine cap nucleotide wherein the cap guanine contains an N7 methylation and the 5′-terminal nucleotide of the mRNA contains a 2-O-methyl. Such a structure is termed the Cap1 structure. This cap results in a higher translational-competency and cellular stability and a reduced activation of cellular pro-inflammatory cytokines, as compared, e.g., to other 5′cap analog structures known in the art. Cap structures include, but are not limited to, 7mG(5′)ppp(5′)N,pN2p (cap 0), 7mG(5′)ppp(5′)N1mpNp (cap 1), and 7mG(5′)-ppp(5′)N1mpN2mp (cap 2).

Because the mRNA contained in an mRNA delivery complex according to any of the embodiments described herein may be capped post-transcriptionally, and because this process is more efficient, nearly 100% of the mRNA may be capped. This is in contrast to about 80% when a cap analog is linked to an mRNA in the course of an in vitro transcription reaction.

According to the present invention, 5′ terminal caps may include endogenous caps or cap analogs. According to the present invention, a 5′ terminal cap may comprise a guanine analog. Useful guanine analogs include, but are not limited to, inosine, N-methyl-guanosine, 2′ fluoro-guanosine, 7-deaza-guanosine, 8-oxo-guanosine, 2-amino-guanosine, LNA-guanosine, and 2-azido-guanosine.

Additional viral sequences such as, but not limited to, the translation enhancer sequence of the barley yellow dwarf virus (BYDV-PAV), the Jaagsiekte sheep retrovirus (JSRV) and/or the Enzootic nasal tumor virus (See e.g., International Pub. No. WO2012129648; herein incorporated by reference in its entirety) can be engineered and inserted in the 3′ UTR of the mRNA of the invention and can stimulate the translation of the construct in vitro and in vivo. Transfection experiments can be conducted in relevant cell lines at and protein production can be assayed by ELISA at 12 hr, 24 hr, 48 hr, 72 hr and day 7 post-transfection.

Further, provided are mRNAs contained in an mRNA delivery complex according to any of the embodiments described herein which may contain an internal ribosome entry site (IRES). First identified as a feature Picorna virus RNA, IRES plays an important role in initiating protein synthesis in absence of the 5′ cap structure. An IRES may act as the sole ribosome binding site, or may serve as one of multiple ribosome binding sites of an mRNA. mRNA containing more than one functional ribosome binding site may encode several peptides or polypeptides that are translated independently by the ribosomes (“multicistronic nucleic acid molecules”). When mRNA are provided with an IRES, further optionally provided is a second translatable region. Examples of IRES sequences that can be used according to the invention include without limitation, those from picomaviruses (e.g. FMDV), pest viruses (CFFV), polio viruses (PV), encephalomyocarditis viruses (ECMV), foot-and-mouth disease viruses (FMDV), hepatitis C viruses (HCV), classical swine fever viruses (CSFV), murine leukemia virus (MLV), simian immune deficiency viruses (SIV) or cricket paralysis viruses (CrPV).

During RNA processing, a long chain of adenine nucleotides (poly-A tail) may be added to a polynucleotide such as an mRNA molecules in order to increase stability. Immediately after transcription, the 3′ end of the transcript may be cleaved to free a 3′ hydroxyl. Then poly-A polymerase adds a chain of adenine nucleotides to the RNA. The process, called polyadenylation, adds a poly-A tail that can be between, for example, approximately 100 and 250 residues long.

Generally, the length of a poly-A tail of the present invention is greater than 30 nucleotides in length. In another embodiment, the poly-A tail is greater than 35 nucleotides in length (e.g., at least or greater than about 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, and 3,000 nucleotides). In some embodiments, the mRNA includes from about 30 to about 3,000 nucleotides (e.g., from 30 to 50, from 30 to 100, from 30 to 250, from 30 to 500, from 30 to 750, from 30 to 1,000, from 30 to 1,500, from 30 to 2,000, from 30 to 2,500, from 50 to 100, from 50 to 250, from 50 to 500, from 50 to 750, from 50 to 1,000, from 50 to 1,500, from 50 to 2,000, from 50 to 2,500, from 50 to 3,000, from 100 to 500, from 100 to 750, from 100 to 1,000, from 100 to 1,500, from 100 to 2,000, from 100 to 2,500, from 100 to 3,000, from 500 to 750, from 500 to 1,000, from 500 to 1,500, from 500 to 2,000, from 500 to 2,500, from 500 to 3,000, from 1,000 to 1,500, from 1,000 to 2,000, from 1,000 to 2,500, from 1,000 to 3,000, from 1,500 to 2,000, from 1,500 to 2,500, from 1,500 to 3,000, from 2,000 to 3,000, from 2,000 to 2,500, and from 2,500 to 3,000).

In one embodiment, the poly-A tail is designed relative to the length of the overall mRNA. This design may be based on the length of the coding region, the length of a particular feature or region (such as the first or flanking regions), or based on the length of the ultimate product expressed from the mRNA.

In this context the poly-A tail may be 10, 20, 30, 40, 50, 60, 70, 80, 90, or 100% greater in length than the mRNA or feature thereof. The poly-A tail may also be designed as a fraction of mRNA to which it belongs. In this context, the poly-A tail may be 10, 20, 30, 40, 50, 60, 70, 80, or 90% or more of the total length of the construct or the total length of the construct minus the poly-A tail. Further, engineered binding sites and conjugation of mRNA for Poly-A binding protein may enhance expression.

Additionally, multiple distinct mRNAs may be linked together to the PABP (Poly-A binding protein) through the 3′-end using modified nucleotides at the 3′-terminus of the poly-A tail. Transfection experiments can be conducted in relevant cell lines at and protein production can be assayed by ELISA at 12 hr, 24 hr, 48 hr, 72 hr and day 7 post-transfection.

The mRNAs of the present invention and the proteins translated from them described herein can be used as therapeutic or prophylactic agents. They are provided for use in medicine. For example, an mRNA described herein can be administered to a subject, wherein the mRNA is translated in vivo to produce a therapeutic or prophylactic polypeptide in the subject. Provided are compositions, methods, kits, and reagents for diagnosis, treatment or prevention of a disease or condition in humans and other mammals. The active therapeutic agents of the invention include mRNA, cells containing polynucleotides, mRNA or polypeptides translated from the mRNA.

In certain embodiments, provided herein are combination therapeutics containing one or more mRNA containing translatable regions that encode for a protein or proteins that boost a mammalian subject's immunity along with a protein that induces antibody-dependent cellular toxicity. For example, provided herein are therapeutics containing one or more nucleic acids that encode trastuzumab and granulocyte-colony stimulating factor (G-CSF). In particular, such combination therapeutics are useful in Her2+ breast cancer patients who develop induced resistance to trastuzumab. (See, e.g., Albrecht, Immunotherapy. 2(6):795-8 (2010)).

Provided herein are methods of inducing translation of a recombinant polypeptide in a cell population using the mRNA described herein. Such translation can be in vivo, ex vivo, in culture, or in vitro. The cell population is contacted with an effective amount of a composition containing a nucleic acid that has at least one nucleoside modification, and a translatable region encoding the recombinant polypeptide. The population is contacted under conditions such that the nucleic acid is localized into one or more cells of the cell population and the recombinant polypeptide is translated in the cell from the nucleic acid.

An “effective amount” of the composition is provided based, at least in part, on the target tissue, target cell type, means of administration, physical characteristics of the nucleic acid (e.g., size, and extent of modified nucleosides), and other determinants. In general, an effective amount of the composition provides efficient protein production in the cell, preferably more efficient than a composition containing a corresponding unmodified nucleic acid. Increased efficiency may be demonstrated by increased cell transfection (i.e., the percentage of cells transfected with the nucleic acid), increased protein translation from the nucleic acid, decreased nucleic acid degradation (as demonstrated, e.g., by increased duration of protein translation from a modified nucleic acid), or reduced innate immune response of the host cell.

Aspects of the invention are directed to methods of inducing in vivo translation of a recombinant polypeptide in a mammalian subject in need thereof. Therein, an effective amount of a composition containing a nucleic acid that has at least one structural or chemical modification and a translatable region encoding the recombinant polypeptide is administered to the subject using the delivery methods described herein. The nucleic acid is provided in an amount and under other conditions such that the nucleic acid is localized into a cell of the subject and the recombinant polypeptide is translated in the cell from the nucleic acid. The cell in which the nucleic acid is localized, or the tissue in which the cell is present, may be targeted with one or more than one rounds of nucleic acid administration.

In certain embodiments, the administered mRNA directs production of one or more recombinant polypeptides that provide a functional activity which is substantially absent in the cell, tissue or organism in which the recombinant polypeptide is translated. For example, the missing functional activity may be enzymatic, structural, or gene regulatory in nature. In related embodiments, the administered mRNA directs production of one or more recombinant polypeptides that increases (e.g., synergistically) a functional activity which is present but substantially deficient in the cell in which the recombinant polypeptide is translated.

In other embodiments, the administered mRNA directs production of one or more recombinant polypeptides that replace a polypeptide (or multiple polypeptides) that is substantially absent in the cell in which the recombinant polypeptide is translated. Such absence may be due to genetic mutation of the encoding gene or regulatory pathway thereof. In some embodiments, the recombinant polypeptide increases the level of an endogenous protein in the cell to a desirable level; such an increase may bring the level of the endogenous protein from a subnormal level to a normal level or from a normal level to a super-normal level.

Alternatively, the recombinant polypeptide functions to antagonize the activity of an endogenous protein present in, on the surface of, or secreted from the cell. Usually, the activity of the endogenous protein is deleterious to the subject: for example, due to mutation of the endogenous protein resulting in altered activity or localization. Additionally, the recombinant polypeptide antagonizes, directly or indirectly, the activity of a biological moiety present in, on the surface of, or secreted from the cell. Examples of antagonized biological moieties include lipids (e.g., cholesterol), a lipoprotein (e.g., low density lipoprotein), a nucleic acid, a carbohydrate, a protein toxin such as shiga and tetanus toxins, or a small molecule toxin such as botulinum, cholera, and diphtheria toxins. Additionally, the antagonized biological molecule may be an endogenous protein that exhibits an undesirable activity, such as a cytotoxic or cytostatic activity.

The recombinant proteins described herein may be engineered for localization within the cell, potentially within a specific compartment such as the nucleus, or are engineered for secretion from the cell or translocation to the plasma membrane of the cell.

In some embodiments, modified mRNAs and their encoded polypeptides in accordance with the present invention may be used for treatment of any of a variety of diseases, disorders, and/or conditions, including but not limited to one or more of the following: autoimmune disorders (e.g. diabetes, lupus, multiple sclerosis, psoriasis, rheumatoid arthritis); inflammatory disorders (e.g. arthritis, pelvic inflammatory disease): infectious diseases (e.g. viral infections (e.g., HIV, HCV, RSV, Chikungunya virus, Zika virus, influenza virus), bacterial infections, fungal infections, sepsis); neurological disorders (e.g. Alzheimer's disease, Huntington's disease; autism; Duchenne muscular dystrophy); cardiovascular disorders (e.g. atherosclerosis, hypercholesterolemia, thrombosis, clotting disorders, angiogenic disorders such as macular degeneration); proliferative disorders (e.g. cancer, benign neoplasms); respiratory disorders (e.g. chronic obstructive pulmonary disease); digestive disorders (e.g. inflammatory bowel disease, ulcers); musculoskeletal disorders (e.g. fibromyalgia, arthritis); endocrine, metabolic, and nutritional disorders (e.g. diabetes, osteoporosis); urological disorders (e.g. renal disease); psychological disorders (e.g. depression, schizophrenia); skin disorders (e.g. wounds, eczema); blood and lymphatic disorders (e.g. anemia, hemophilia); etc.

Diseases characterized by dysfunctional or aberrant protein activity include cystic fibrosis, sickle cell anemia, epidermolysis bullosa, amyotrophic lateral sclerosis, and glucose-6-phosphate dehydrogenase deficiency. The present invention provides a method for treating such conditions or diseases in a subject by introducing nucleic acid or cell-based therapeutics containing the mRNA provided herein, wherein the mRNA encode for a protein that antagonizes or otherwise overcomes the aberrant protein activity present in the cell of the subject. Specific examples of a dysfunctional protein are the missense mutation variants of the cystic fibrosis transmembrane conductance regulator (CFTR) gene, which produce a dysfunctional protein variant of CFTR protein, which causes cystic fibrosis.

Diseases characterized by missing (or substantially diminished such that proper (normal or physiological protein function does not occur) protein activity include cystic fibrosis, Niemann-Pick type C, .beta. thalassemia major, Duchenne muscular dystrophy, Hurler Syndrome, Hunter Syndrome, and Hemophilia A. Such proteins may not be present, or are essentially non-functional. The present invention provides a method for treating such conditions or diseases in a subject by introducing nucleic acid or cell-based therapeutics containing the mRNA provided herein, wherein the mRNA encode for a protein that replaces the protein activity missing from the target cells of the subject. Specific examples of a dysfunctional protein are the nonsense mutation variants of the cystic fibrosis transmembrane conductance regulator (CFTR) gene, which produce a nonfunctional protein variant of CFTR protein, which causes cystic fibrosis.

Thus, provided are methods of treating cystic fibrosis in a mammalian subject by contacting a cell of the subject with an mRNA having a translatable region that encodes a functional CFTR polypeptide, under conditions such that an effective amount of the CTFR polypeptide is present in the cell. Preferred target cells are epithelial, endothelial and mesothelial cells, such as the lung, and methods of administration are determined in view of the target tissue; i.e., for lung delivery, the RNA molecules are formulated for administration by inhalation.

In another embodiment, the present invention provides a method for treating hyperlipidemia in a subject, by introducing into a cell population of the subject with a modified mRNA molecule encoding Sortilin, a protein recently characterized by genomic studies, thereby ameliorating the hyperlipidemia in a subject. The SORT1 gene encodes a trans-Golgi network (TGN) transmembrane protein called Sortilin. Genetic studies have shown that one of five individuals has a single nucleotide polymorphism, rs2740374, in the 1p13 locus of the SORT1 gene that predisposes them to having low levels of low-density lipoprotein (LDL) and very-low-density lipoprotein (VLDL). Each copy of the minor allele, present in about 30% of people, alters LDL cholesterol by 8 mg/dL, while two copies of the minor allele, present in about 5% of the population, lowers LDL cholesterol 16 mg/dL. Carriers of the minor allele have also been shown to have a 40% decreased risk of myocardial infarction. Functional in vivo studies in mice describes that overexpression of SORT1 in mouse liver tissue led to significantly lower LDL-cholesterol levels, as much as 80% lower, and that silencing SORT increased LDL cholesterol approximately 200% (Musunuru K et al. From noncoding variant to phenotype via SORT1 at the 1p13 cholesterol locus. Nature 2010; 466: 714-721.

In another embodiment, the present invention provides a method for treating hematopoietic disorders, cardiovascular disease, oncology, diabetes, cystic fibrosis, neurological diseases, inborn errors of metabolism, skin and systemic disorders, and blindness. The identity of molecular targets to treat these specific diseases has been described (Templeton ed., Gene and Cell Therapy: Therapeutic Mechanisms and Strategies, 3.sup.rd Edition, Bota Raton, Fla.: CRC Press; herein incorporated by reference in its entirety).

Provided herein, are methods to prevent infection and/or sepsis in a subject at risk of developing infection and/or sepsis, the method comprising administering to a subject in need of such prevention a composition comprising an mRNA precursor encoding an anti-microbial polypeptide (e.g., an anti-bacterial polypeptide), or a partially or fully processed form thereof in an amount sufficient to prevent infection and/or sepsis. In certain embodiments, the subject at risk of developing infection and/or sepsis may be a cancer patient. In certain embodiments, the cancer patient may have undergone a conditioning regimen. In some embodiments, the conditioning regiment may include, but is not limited to, chemotherapy, radiation therapy, or both. As a non-limiting example, an mRNA can encode Protein C, its zymogen or prepro-protein, the activated form of Protein C (APC) or variants of Protein C which are known in the art. In some embodiments, the mRNA is chemically modified and delivered to cells. Non-limiting examples of polypeptides which may be encoded within the chemically modified mRNAs of the present invention include those taught in U.S. Pat. Nos. 7,226,999; 7,498,305; 6,630,138 each of which is incorporated herein by reference in its entirety. These patents teach Protein C like molecules, variants and derivatives, any of which may be encoded within the chemically modified molecules of the present invention.

Further provided herein, are methods to treat infection and/or sepsis in a subject, the method comprising administering to a subject in need of such treatment a composition comprising an mRNA precursor encoding an anti-microbial polypeptide (e.g., an anti-bacterial polypeptide), e.g., an anti-microbial polypeptide described herein, or a partially or fully processed form thereof in an amount sufficient to treat an infection and/or sepsis. In certain embodiments, the subject in need of treatment is a cancer patient. In certain embodiments, the cancer patient has undergone a conditioning regimen. In some embodiments, the conditioning regiment may include, but is not limited to, chemotherapy, radiation therapy, or both.

In certain embodiments, the subject may exhibits acute or chronic microbial infections (e.g., bacterial infections). In certain embodiments, the subject may have received or may be receiving a therapy. In certain embodiments, the therapy may include, but is not limited to, radiotherapy, chemotherapy, steroids, ultraviolet radiation, or a combination thereof. In certain embodiments, the patient may suffer from a microvascular disorder. In some embodiments, the microvascular disorder may be diabetes. In certain embodiments, the patient may have a wound. In some embodiments, the wound may be an ulcer. In a specific embodiment, the wound may be a diabetic foot ulcer. In certain embodiments, the subject may have one or more burn wounds. In certain embodiments, the administration may be local or systemic. In certain embodiments, the administration may be subcutaneous. In certain embodiments, the administration may be intravenous. In certain embodiments, the administration may be oral. In certain embodiments, the administration may be topical. In certain embodiments, the administration may be by inhalation. In certain embodiments, the administration may be rectal. In certain embodiments, the administration may be vaginal.

Other aspects of the present disclosure relate to transplantation of cells containing mRNA to a mammalian subject. Administration of cells to mammalian subjects is known to those of ordinary skill in the art, and include, but is not limited to, local implantation (e.g., topical or subcutaneous administration), organ delivery or systemic injection (e.g., intravenous injection or inhalation), and the formulation of cells in pharmaceutically acceptable carrier. Such compositions containing mRNA can be formulated for administration intramuscularly, transarterially, intraperitoneally, intravenously, intranasally, subcutaneously, endoscopically, transdermally, or intrathecally. In some embodiments, the composition may be formulated for extended release.

The subject to whom the therapeutic agent may be administered suffers from or may be at risk of developing a disease, disorder, or deleterious condition. Provided are methods of identifying, diagnosing, and classifying subjects on these bases, which may include clinical diagnosis, biomarker levels, genome-wide association studies (GWAS), and other methods known in the art.

The mRNA of the present invention may be used for wound treatment, e.g. of wounds exhibiting delayed healing. Provided herein are methods comprising the administration of mRNA in order to manage the treatment of wounds. The methods herein may further comprise steps carried out either prior to, concurrent with or post administration of the mRNA. For example, the wound bed may need to be cleaned and prepared in order to facilitate wound healing and hopefully obtain closure of the wound. Several strategies may be used in order to promote wound healing and achieve wound closure including, but not limited to: (i) debridement, optionally repeated, sharp debridement (surgical removal of dead or infected tissue from a wound), optionally including chemical debriding agents, such as enzymes, to remove necrotic tissue; (ii) wound dressings to provide the wound with a moist, warm environment and to promote tissue repair and healing.

Examples of materials that are used in formulating wound dressings include, but are not limited to: hydrogels (e.g., AQUASORB®; DUODERM®), hydrocolloids (e.g., AQUACEL®; COMFEEL®), foams (e.g., LYOFOAM®; SPYROSORB®), and alginates (e.g., ALGISITE®; CURASORB®); (iii) additional growth factors to stimulate cell division and proliferation and to promote wound healing e.g. becaplermin (REGRANEX GEL®), a human recombinant platelet-derived growth factor that is approved by the FDA for the treatment of neuropathic foot ulcers; (iv) soft-tissue wound coverage, a skin graft may be necessary to obtain coverage of clean, non-healing wounds. Examples of skin grafts that may be used for soft-tissue coverage include, but are not limited to: autologous skin grafts, cadaveric skin graft, bioengineered skin substitutes (e.g., APLIGRAF™; DERMAGRAFT™).

In certain embodiments, the mRNA of the present invention may further include hydrogels (e.g., AQUASORB™; DUODERM™), hydrocolloids (e.g., AQUACEL™; COMFEEL™), foams (e.g., LYOFOAM™; SPYROSORB™), and/or alginates (e.g., ALGISITE™; CURASORB™). In certain embodiments, the mRNA of the present invention may be used with skin grafts including, but not limited to, autologous skin grafts, cadaveric skin graft, or bioengineered skin substitutes (e.g., APLIGRAF™; DERMAGRAFT™). In some embodiments, the mRNA may be applied with would dressing formulations and/or skin grafts or they may be applied separately but methods such as, but not limited to, soaking or spraying.

In some embodiments, compositions for wound management may comprise an mRNA encoding for an anti-microbial polypeptide (e.g., an anti-bacterial polypeptide) and/or an anti-viral polypeptide. A precursor or a partially or fully processed form of the anti-microbial polypeptide may be encoded. The composition may be formulated for administration using a bandage (e.g., an adhesive bandage). The anti-microbial polypeptide and/or the anti-viral polypeptide may be intermixed with the dressing compositions or may be applied separately, e.g., by soaking or spraying.

In one embodiment of the invention, the mRNA may encode antibodies and fragments of such antibodies. These may be produced by any one of the methods described herein. The antibodies may be of any of the different subclasses or isotypes of immunoglobulin such as, but not limited to, IgA, IgG, or IgM, or any of the other subclasses. Exemplary antibody molecules and fragments that may be prepared according to the invention include, but are not limited to, immunoglobulin molecules, substantially intact immunoglobulin molecules and those portions of an immunoglobulin molecule that may contain the paratope. Such portion of antibodies that contain the paratope include, but are not limited to Fab, Fab′, F(ab′).sub.2, F(v) and those portions known in the art.

The polynucleotides of the invention may encode variant antibody polypeptides which may have a certain identity with a reference polypeptide sequence, or have a similar or dissimilar binding characteristic with the reference polypeptide sequence.

Antibodies obtained by the methods of the present invention may be chimeric antibodies comprising non-human antibody-derived variable region(s) sequences, derived from the immunized animals, and human antibody-derived constant region(s) sequences. In addition, they can also be humanized antibodies comprising complementary determining regions (CDRs) of non-human antibodies derived from the immunized animals and the framework regions (FRs) and constant regions derived from human antibodies. In another embodiment, the methods provided herein may be useful for enhancing antibody protein product yield in a cell culture process.

In one embodiment, provided are methods for treating or preventing a microbial infection (e.g., a bacterial infection) and/or a disease, disorder, or condition associated with a microbial or viral infection, or a symptom thereof, in a subject, by administering an mRNA encoding an anti-microbial polypeptide. Said administration may be in combination with an anti-microbial agent (e.g., an anti-bacterial agent), e.g., an anti-microbial polypeptide or a small molecule anti-microbial compound described herein. The anti-microbial agents include, but are not limited to, anti-bacterial agents, anti-viral agents, anti-fungal agents, anti-protozoal agents, anti-parasitic agents, and anti-prion agents.

The agents can be administered simultaneously, for example in a combined unit dose (e.g., providing simultaneous delivery of both agents). The agents can also be administered at a specified time interval, such as, but not limited to, an interval of minutes, hours, days or weeks. Generally, the agents may be concurrently bioavailable, e.g., detectable, in the subject. In some embodiments, the agents may be administered essentially simultaneously, for example two unit dosages administered at the same time, or a combined unit dosage of the two agents. In other embodiments, the agents may be delivered in separate unit dosages. The agents may be administered in any order, or as one or more preparations that includes two or more agents. In a preferred embodiment, at least one administration of one of the agents, e.g., the first agent, may be made within minutes, one, two, three, or four hours, or even within one or two days of the other agent, e.g., the second agent. In some embodiments, combinations can achieve synergistic results, e.g., greater than additive results, e.g., at least 25, 50, 75, 100, 200, 30, 400, or 500% greater than additive results.

Diseases, disorders, or conditions which may be associated with bacterial infections include, but are not limited to one or more of the following: abscesses, actinomycosis, acute prostatitis, Aeromonas hydrophila, annual ryegrass toxicity, anthrax, bacillary peliosis, bacteremia, bacterial gastroenteritis, bacterial meningitis, bacterial pneumonia, bacterial vaginosis, bacterium-related cutaneous conditions, bartonellosis, BCG-oma, botryomycosis, botulism, Brazilian purpuric fever, Brodie abscess, brucellosis Buruli ulcer, campylobacteriosis, caries, Carrion's disease, cat scratch disease, cellulitis, chlamydia infection, cholera, chronic bacterial prostatitis, chronic recurrent multifocal osteomyelitis, clostridial necrotizing enteritis, combined periodontic-endodontic lesions, contagious bovine pleuropneumonia, diphtheria, diphtheritic stomatitis, ehrlichiosis, erysipelas, piglottitis, erysipelas, Fit-Hugh-Curtis syndrome, flea-borne spotted fever, foot rot (infectious pododermatitis), Garre's sclerosing osteomyelitis, Gonorrhea. Granuloma inguinale, human granulocytic anaplasmosis, human monocytotropic ehrlichiosis, hundred days' cough, impetigo, late congenital syphilitic oculopathy, legionellosis, Lemierre's syndrome, leprosy (Hansen's Disease), leptospirosis, listeriosis, Lyme disease, lymphadenitis, melioidosis, meningococcal disease, meningococcal septicaemia, methicillin-resistant Staphylococcus aureus (MRSA) infection, Mycobacterium avium-intracellulare (MAI), Mycoplasma pneumonia, necrotizing fasciitis, nocardiosis, noma (cancrum oris or gangrenous stomatitis), omphalitis, orbital cellulitis, osteomelitis, overwhelming post-splenectomy infection (OPSI), ovine brucellosis, pasteurellosis, periorbital cellulitis, pertussis (whooping cough), plague, pneumococcal pneumonia, Pott disease, proctitis, pseudomonas infection, psittacosis, pyaemia, pyomyositis, Q fever, relapsing fever (typhinia), rheumatic fever, Rocky Mountain spotted fever (RMSF), rickettsiosis, Salmonellosis, scarlet fever, sepsis, Serratia infection, shigellosis, southern tick-associated rash illness, staphylococcal scalded skin syndrome, streptococcal pharyngitis, swimming pool granuloma, swine brucellosis, syphilis, syphilitic aortitis, tetanus, toxic shock syndrome (TSS), trachoma, trench fever, tropical ulcer, tuberculosis, tularemia, typhoid fever, typhus, urogenital tuberculosis, urinary tract infections, vancomycin-resistant Staphylococcus aureus infection, Waterhouse-Friderichsen syndrome, Pseudotuberculosis (Yersinia) disease, and Yersiniosis. Other diseases, disorders, and/or conditions associated with bacterial infections can include, for example, Alzheimer's disease, anorexia nervosa, asthma, atherosclerosis, attention deficit hyperactivity disorder, autism, autoimmune diseases, bipolar disorder, cancer (e.g., colorectal cancer, gallbladder cancer, lung cancer, pancreatic cancer, and stomach cancer), chronic fatigue syndrome, chronic obstructive pulmonary disease, Crohn's disease, coronary heart disease, dementia, depression. Guillain-Barre syndrome, metabolic syndrome, multiple sclerosis, myocardial infarction, obesity, obsessive-compulsive disorder, panic disorder, psoriasis, rheumatoid arthritis, sarcoidosis, schizophrenia, stroke, thromboangiitis obliterans (Buerger's disease), and Tourette syndrome.

The bacterium described herein can be a Gram-positive bacterium or a Gram-negative bacterium. Bacterial pathogens include, but are not limited to, Acinetobacter baumannii, Bacillus anthracis, Bacillus subtilis, Bordetella pertussis, Borrelia burgdorferi, Brucella abortus, Brucella canis, Brucella melitensis, Brucella suis, Campylobacter jejuni, Chlamydia pneumoniae, Chlamydia trachomatis, Chlamydophila psittaci, Clostridium botulinum, Clostridium difficile, Clostridium perfringens, Clostridium tetani, coagulase Negative Staphylococcus, Corynebacterium diphtheria, Enterococcus faecalis, Enterococcus faecium, Escherichia coli, enterotoxigenic Escherichia coli (ETEC), enteropathogenic E. coli, E. coli O157:H7, Enterobacter sp., Francisella tularensis, Haemophilus influenzae, Helicobacter pylori, Klebsiella pneumoniae, Legionella pneumophila, Leptospira interrogans, Listeria monocytogenes, Moraxella catarralis, Mycobacterium leprae, Mycobacterium tuberculosis, Mycoplasma pneumoniae, Neisseria gonorrhoeae, Neisseria meningitides, Preteus mirabilis, Proteus sps., Pseudomonas aeruginosa, Rickettsia rickettsii, Salmonella typhi, Salmonella typhimurium, Serratia marcesens, Shigella flexneri, Shigella sonnei, Staphylococcus aureus, Staphylococcus epidermidis, Staphylococcus saprophyticus, Streptococcus agalactiae, Streptococcus mutans, Streptococcus pneumoniae, Streptococcus pyogenes, Treponema pallidum, Vibrio cholerae, and Yersinia pestis. Bacterial pathogens may also include bacteria that cause resistant bacterial infections, for example, clindamycin-resistant Clostridium difficile, fluoroquinolon-resistant Clostridium difficile, methicillin-resistant Staphylococcus aureus (MRSA), multidrug-resistant Enterococcus faecalis, multidrug-resistant Enterococcus faecium, multidrug-resistance Pseudomonas aeruginosa, multidrug-resistant Acinetobacter baumannii, and vancomycin-resistant Staphylococcus aureus (VRSA).

In one embodiment, the modified mRNA of the present invention may be administered in conjunction with one or more antibiotics. These include, but are not limited to Aknilox, Ambisome, Amoxycillin, Ampicillin, Augmentin, Avelox, Azithromycin, Bactroban, Betadine, Betnovate, Blephamide, Cefaclor, Cefadroxil, Cefdinir, Cefepime, Cefix, Cefixime, Cefoxitin, Cefpodoxime, Cefprozil, Cefuroxime, Cefzil, Cephalexin, Cephazolin, Ceptaz, Chloramphenicol, Chlorhexidine, Chloromycetin, Chlorsig, Ciprofloxacin, Clarithromycin, Clindagel, Clindamycin, Clindatech, Cloxacillin, Colistin, Co-trimoxazole, Demeclocycline, Diclocil, Dicloxacillin, Doxycycline, Duricef, Erythromycin, Flamazine, Floxin, Framycetin, Fucidin, Furadantin, Fusidic, Gatifloxacin, Gemifloxacin, Gemifloxacin, llosone, Iodine, Levaquin, Levofloxacin, Lomefloxacin, Maxaquin, Mefoxin, Meronem, Minocycline, Moxifloxacin, Myambutol, Mycostatin, Neosporin, Netromycin, Nitrofurantoin, Norfloxacin, Norilet, Ofloxacin, Omnicef, Ospamox, Oxytetracycline, Paraxin, Penicillin, Pneumovax, Polyfax, Povidone, Rifadin, Rifampin, Rifaximin, Rifinah, Rimactane, Rocephin, Roxithromycin, Serormcin, Soframycin, Sparfloxacin, Staphlex, Targocid, Tetracycline, Tetradox, Tetralysal, tobramycin, Tobramycin, Trecator, Tygacil, Vancocin, Velosef, Vibramycin, Xifaxan, Zagam, Zitrotek, Zoderm, Zymar, and Zyvox.

Exemplary anti-bacterial agents include, but are not limited to, aminoglycosides (e.g., amikacin (AMIKIN™), gentamicin (GARAMYCIN™), kanamycin (KANTREX™), neomycin (MYCIFRADIN™), netilmicin (NETROMYCIN™), tobramycin (NEBCIN™), Paromomycin (HUMATIN™), ansamycins (e.g., geldanamycin, herbimycin), carbacephem (e.g., loracarbef (LORABID™), Carbapenems (e.g., ertapenem (INVANZ™), doripenem (DORIBAX™), imipenem/cilastatin (PRIMAXIN™), meropenem (MERREM™), cephalosporins (first generation) (e.g., cefadroxil (DURICEF™), cefazolin (ANCEF™), cefalotin or cefalothin (KEFLIN™), cefalexin (KEFLEX™), cephalosporins (second generation) (e.g., cefaclor (CECLOR™), cefamandole (MANDOL™), cefoxitin (MEFOXIN™), cefprozil (CEFZIL™), cefuroxime (CEFTIN™, ZINNAT™), cephalosporins (third generation) (e.g., cefixime (SUPRAX™), cefdinir (OMNICEF™, CEFDIEL™), cefditoren (SPECTRACEF™), cefoperazone (CEFOBID™), cefotaxime (CLAFORAN™), cefpodoxime (VANTIN™), ceftazidime (FORTAZ™), ceftibuten (CEDAX™), ceftizoxime (CEFIZOX™), ceftriaxone (ROCEPHIN™), cephalosporins (fourth generation) (e.g., cefepime (MAXIPIME™), cephalosporins (fifth generation) (e.g., ceftobiprole (ZEFTERA™), glycopeptides (e.g., teicoplanin (TARGOCID™), vancomycin (VANCOCIN™), telavancin (VIBATIV™), lincosamides (e.g., clindamycin (CLEOCIN™), lincomycin (LINCOCIN™), lipopeptide (e.g., daptomycin (CUBICIN™), macrolides (e.g., azithromycin (ZITHROMAX™, SUMAMED™, ZITROCIN™), clarithromycin (BIAXIN™), dirithromycin (DYNABAC™), erythromycin (ERYTHOCIN™, ERYTHROPED™), roxithromycin, troleandomycin (TAO™), telithromycin (KETEK™), spectinomycin (TROBICIN™), monobactams (e.g., aztreonam (AZACTAM™), nitrofurans (e.g., furazolidone (FUROXONE™), nitrofurantoin (MACRODANTIN™, MACROBID™), penicillins (e.g., amoxicillin (NOVAMOX™, AMOXIL™), ampicillin (PRINCIPEN™), azlocillin, carbenicillin (GEOCILLIN™), cloxacillin (TEGOPEN™), dicloxacillin (DYNAPEN™), flucloxacillin (FLOXAPEN™), mezlocillin (MEZLIN™), methicillin (STAPHCILLIN™), nafcillin (UNIPEN™), oxacillin (PROSTAPHLIN™), penicillin G (PENTIDS™), penicillin V (PEN-VEE-K™), piperacillin (PIPRACIL™), temocillin (NEGABAN™), ticarcillin (TICAR™), penicillin combinations (e.g., amoxicillin/clavulanate (AUGMENTIN™), ampicillin/sulbactam (UNASYN™), piperacillin/tazobactam (ZOSYN™), ticarcillin/clavulanate (TIMENTIN™), polypeptides (e.g., bacitracin, colistin (COLY-MYCIN-S™), polymyxin B, quinolones (e.g., ciprofloxacin (CIPRO™, CIPROXIN™, CIPROBAY™), enoxacin (PENETREX™), gatifloxacin (TEQUIN™), levofloxacin (LEVAQUIN™), lomefloxacin (MAXAQUIN™), moxifloxacin (AVELOX™), nalidixic acid (NEGGRAM™), norfloxacin (NOROXIN™), ofloxacin (FLOXIN™, OCUFLOX™), trovafloxacin (TROVAN™), grepafloxacin (RAXAR™), sparfloxacin (ZAGAM™), temafloxacin (OMNIFLOX™), sulfonamides (e.g., mafenide (SULFAMYLON™), sulfonamidochrysoidine (PRONTOSIL™), sulfacetamide (SULAMYD™, BLEPH-100), sulfadiazine (MICRO-SULFON™), silver sulfadiazine (SILVADENE™), sulfamethizole (THIOSULFIL FORTE™), sulfamethoxazole (GANTANOL™), sulfanilimide, sulfasalazine (AZULFIDINE™), sulfisoxazole (GANTRISIN™), trimethoprim (PROLOPRIM™), TRIMPEX™), trimethoprim-sulfamethoxazole (co-trimoxazole) (TMP-SMX) (BACTRIM™, SEPTRA™), tetracyclines (e.g., demeclocycline (DECLOMYCIN™), doxycycline (VIBRAMYCIN™), minocycline (MINOCIN™), oxytetracycline (TERRAMYCIN™), tetracycline (SUMYCIN™, ACHROMYCIN™ V, STECLIN™)), drugs against mycobacteria (e.g., clofazimine (LAMPRENE™), dapsone (AVLOSULFON™), capreomycin (CAPASTAT™), cycloserine (SEROMYCIN™), ethambutol (MYAMBUTOL™), ethionamide (TRECATOR™), isoniazid (I.N.H.™), pyrazinamide (ALDINAMIDE™), rifampin (RIFADIN™, RIMACTANE™), rifabutin (MYCOBUTIN™), rifapentine (PRIFTIN™), streptomycin), and others (e.g., arsphenamine (SALVARSAN™), chloramphenicol (CHLOROMYCETIN™), fosfomycin (MONUROL™), fusidic acid (FUCIDIN™), linezolid (ZYVOX™), metronidazole (FLAGYL™), mupirocin (BACTROBAN™), platensimycin, quinupristin/dalfopristin (SYNERCID™), rifaximin (XIFAXAN™), thiamphenicol, tigecycline (TIGACYL™), timidazole (TINDAMAX™, FASIGYN™)).

In another embodiment, provided are methods for treating or preventing a viral infection and/or a disease, disorder, or condition associated with a iral infection, or a symptom thereof, in a subject, by administering an mRNA encoding an anti-viral polypeptide, e.g., an anti-viral polypeptide described herein in combination with an anti-viral agent, e.g., an anti-viral polypeptide or a small molecule anti-viral agent described herein.

Diseases, disorders, or conditions associated with viral infections include, but are not limited to, acute febrile pharyngitis, pharyngoconjunctival fever, epidemic keratoconjunctivitis, infantile gastroenteritis, Coxsackie infections, infectious mononucleosis, Burkitt lymphoma, acute hepatitis, chronic hepatitis, hepatic cirrhosis, hepatocellular carcinoma, primary HSV-1 infection (e.g., gingivostomatitis in children, tonsillitis and pharyngitis in adults, keratoconjunctivitis), latent HSV-1 infection (e.g., herpes labialis and cold sores), primary HSV-2 infection, latent HSV-2 infection, aseptic meningitis, infectious mononucleosis, Cytomegalic inclusion disease, Kaposi sarcoma, multicentric Castleman disease, primary effusion lymphoma, AIDS, influenza, Reve syndrome, measles, postinfectious encephalomyelitis, Mumps, hyperplastic epithelial lesions (e.g., common, flat, plantar and anogenital warts, laryngeal papillomas, epidermodysplasia verruciformis), cervical carcinoma, squamous cell carcinomas, croup, pneumonia, bronchiolitis, common cold, Poliomyelitis, Rabies, bronchiolitis, pneumonia, influenza-like syndrome, severe bronchiolitis with pneumonia, German measles, congenital rubella, Varicella, and herpes zoster.

Viral pathogens include, but are not limited to, adenovirus, coxsackievirus, dengue virus, encephalitis virus, Epstein-Barr virus, hepatitis A virus, hepatitis B virus, hepatitis C virus, herpes simplex virus type 1, herpes simplex virus type 2, cytomegalovirus, human herpesvirus type 8, human immunodeficiency virus, influenza virus, measles virus, mumps virus, human papillomavirus, parainfluenza virus, poliovirus, rabies virus, respiratory syncytial virus, rubella virus, varicella-zoster virus, West Nile virus, and yellow fever virus. Viral pathogens may also include viruses that cause resistant viral infections.

Exemplary anti-viral agents include, but are not limited to, abacavir (ZIAGEN™), abacavir/lamivudine/zidovudine (Trizivir™), aciclovir or acyclovir (CYCLOVIR™, HERPEX™, ACIVIR™, ACIVIRAX™, ZOVIRAX™, ZOVIR™), adefovir (Preveon™, Hepsera™), amantadine (SYMMETREL™), amprenavir (AGENERASE™), ampligen, arbidol, atazanavir (REYATAZ™), boceprevir, cidofovir, darunavir (PREZISTA™), delavirdine (RESCRIPTOR™), didanosine (VIDEX™), docosanol (ABREVA™), edoxudine, efavirenz (SUSTIVA™, STOCRIN™), emtricitabine (EMTRIVA™), emtricitabine/tenofovir/efavirenz (ATRIPLA™), enfuvirtide (FUZEON™), entecavir (BARACLUDE™, ENTAVIR™), famciclovir (FAMVIR™), fomivirsen (VITRAVENE™), fosamprenavir (LEXIVA™, TELZIR™), foscamet (FOSCAVIR™), fosfonet, ganciclovir (CYTOVENE™, CYMEVENE™, VITRASERT™), GS 9137 (ELVITEGRAVIR™), imiquimod (ALDARA™, ZYCLARA™, BESELNA™), indinavir (CRIXIVAN™), inosine, inosine pranobex (IMUNOVIR™), interferon type I, interferon type II, interferon type III, kutapressin (NEXAVIR™), lamivudine (ZEFFIX™, HEPTOVIR™, EPIVIR™), lamivudine/zidovudine (COMBIVIR™), lopinavir, loviride, maraviroc (SELZENTRY™, CELSENTRI™), methisazone, MK-2048, moroxydine, nelfinavir (VIRACEPT™), nevirapine (VIRAMUNE™), oseltamivir (TAMIFLU™), peginterferon alfa-2a (PEGASYS™), penciclovir (DENAVIR™), peramivir, pleconaril, podophyllotoxin (CONDYLOX™), raltegravir (ISENTRESS™), ribavirin (COPEGUs™, REBETOL™, RIBASPHERE™, VILONA™ AND VIRAZOLE™), rimantadine (FLUMADINE™), ritonavir (NORVIR™), pyramidine, saquinavir (INVIRASE™, FORTOVASE™), stavudine, tea tree oil (melaleuca oil), tenofovir (VIREAD™), tenofovir/emtricitabine (TRUVADA™), tipranavir (APTIVUS™), trifluridine (VIROPTIC™), tromantadine (VIRU-MERZ™), valaciclovir (VALTREX™), valganciclovir (VALCYTE™), vicriviroc, vidarabine, viramidine, zalcitabine, zanamivir (RELENZA™), and zidovudine (azidothymidine (AZT), RETROVIR™, RETROVIS™).

Diseases, disorders, or conditions associated with fungal infections include, but are not limited to, aspergilloses, blastomycosis, candidasis, coccidioidomycosis, cryptococcosis, histoplasmosis, mycetomas, paracoccidioidomycosis, and tinea pedis. Furthermore, persons with immuno-deficiencies are particularly susceptible to disease by fungal genera such as Aspergillus, Candida, Cryptoccocus, Histoplasma, and Pneumocystis. Other fungi can attack eyes, nails, hair, and especially skin, the so-called dermatophytic fungi and keratinophilic fungi, and cause a variety of conditions, of which ringworms such as athlete's foot are common. Fungal spores are also a major cause of allergies, and a wide range of fungi from different taxonomic groups can evoke allergic reactions in some people.

Fungal pathogens include, but are not limited to, Ascomycota (e.g., Fusarium oxysporum, Pneumocystis jirovecii, Aspergillus spp., Coccidioides immitis/posadasii, Candida albicans), Basidiomycota (e.g., Filobasidiella neoformans, Trichosporon), Microsporidia (e.g., Encephalitozoon cuniculi, Enterocytozoon bieneusi), and Mucoromycotina (e.g., Mucor circinelloides, Rhizopus oryzae, Lichtheimia corymbifera).

Exemplary anti-fungal agents include, but are not limited to, polyene antifungals (e.g., natamycin, rimocidin, filipin, nystatin, amphotericin B, candicin, hamycin), imidazole antifungals (e.g., miconazole (MICATIN™, DAKTARIN™), ketoconazole (NIZORAL™, FUNGORAL™, SEBIZOLE™), clotrimazole (LOTRIMIN™, LOTRIMIN™ AF, CANESTEN™), econazole, omoconazole, bifonazole, butoconazole, fenticonazole, isoconazole, oxiconazole, sertaconazole (ERTACZO™), sulconazole, tioconazole), triazole antifungals (e.g., albaconazole fluconazole, itraconazole, isavuconazole, ravuconazole, posaconazole, voriconazole, terconazole), thiazole antifungals (e.g., abafungin), allylamines (e.g., terbinafine (LAMISIL™), naftifine (NAFTIN™), butenafine (LOTRIMIN™ Ultra)), echinocandins (e.g., anidulafungin, caspofungin, micafungin), and others (e.g., polygodial, benzoic acid, ciclopirox, tolnaflate (TINACTIN™, DESENEX™, AFTATE™), undecylenic acid, flucytosine or 5-fluorocytosine, griseofulvin, haloprogin, sodium bicarbonate, allicin).

Diseases, disorders, or conditions associated with protozoal infections include, but are not limited to, amoebiasis, giardiasis, trichomoniasis, African Sleeping Sickness, American Sleeping Sickness, leishmaniasis (Kala-Azar), balantidiasis, toxoplasmosis, malaria, Acanthamoeba keratitis, and babesiosis.

Protozoal pathogens include, but are not limited to, Entamoeba histolytica, Giardia lambila, Trichomonas vaginalis, Trypanosoma brucei, T. cruzi, Leishmania donovani, Balantidium coli, Toxoplasma gondii, Plasmodium spp., and Babesia microti.

Exemplary anti-protozoal agents include, but are not limited to, eflomithine, furazolidone (FUROXONE™, DEPENDAL-M™), melarsoprol, metronidazole (FLAGYL™), ornidazole, paromomycin sulfate (HUMATIN™), pentamidine, pyrimethamine (DARAPRIM™), and timidazole (TINDAMAX™, FASIGYN™).

Diseases, disorders, or conditions associated with parasitic infections include, but are not limited to, acanthamoeba keratitis, amoebiasis, ascariasis, babesiosis, balantidiasis, baylisascariasis, chagas disease, clonorchiasis, cochliomyia, cryptosporidiosis, diphyllobothriasis, dracunculiasis, echinococcosis, elephantiasis, enterobiasis, fascioliasis, fasciolopsiasis, filariasis, giardiasis, gnathostomiasis, hymenolepiasis, isosporiasis, katayama fever, leishmaniasis, lyme disease, malaria, metagonimiasis, myiasis, onchocerciasis, pediculosis, scabies, schistosomiasis, sleeping sickness, strongyloidiasis, taeniasis, toxocariasis, toxoplasmosis, trichinosis, and trichuriasis.

Parasitic pathogens include, but are not limited to, Acanthamoeba, Anisakis, Ascaris lumbricoides, botfly, Balantidium coli, bedbug, Cestoda, chiggers, Cochliomyia hominivorax, Entamoeba histolytica, Fasciola hepatica, Giardia lamblia, hookworm, Leishmania, Linguatula serrata, liver fluke, Loa boa, Paragonimus, pinworm, Plasmodium falciparum, Schistosoma, Strongyloides stercoralis, mite, tapeworm, Toxoplasma gondii, Trypanosoma, whipworm, Wuchereria bancrofti.

Exemplary anti-parasitic agents include, but are not limited to, antinematodes (e.g., mebendazole, pyrantel pamoate, thiabendazole, diethylcarbamazine, ivermectin), anticestodes (e.g., niclosamide, praziquantel, albendazole), antitrematodes (e.g., praziquantel), antiamoebics (e.g., rifampin, amphotericin B), and antiprotozoals (e.g., melarsoprol, eflomithine, metronidazole, timidazole).

Diseases, disorders, or conditions associated with prion infections include, but are not limited to Creutzfeldt-Jakob disease (CJD), iatrogenic Creutzfeldt-Jakob disease (iCJD), variant Creutzfeldt-Jakob disease (vCJD), familial Creutzfeldt-Jakob disease (fCJD), sporadic Creutzfeldt-Jakob disease (sCJD), Gerstmann-Stra ussler-Scheinker syndrome (GSS), fatal familial insomnia (FFI), Kuru, Scrapie, bovine spongiform encephalopathy (BSE), mad cow disease, transmissible mink encephalopathy (TME), chronic wasting disease (CWD), feline spongiform encephalopathy (FSE), exotic ungulate encephalopathy (EUE), and spongiform encephalopathy.

Exemplary anti-prion agents include, but are not limited to, flupirtine, pentosan polysuphate, quinacrine, and tetracyclic compounds.

As described herein, a useful feature of the mRNA of the invention is the capacity to modulate (e.g., reduce, evade or avoid) the innate immune response of a cell. In one aspect, provided herein are mRNA encoding a polypeptide of interest which when delivered to cells, results in a reduced immune response from the host as compared to the response triggered by a reference compound, e.g. an unmodified polynucleotide corresponding to an mRNA of the invention, or a different mRNA of the invention. As used herein, a “reference compound” is any molecule or substance which when administered to a mammal, results in an innate immune response having a known degree, level or amount of immune stimulation. A reference compound need not be a nucleic acid molecule and it need not be any of the mRNA of the invention. Hence, the measure of a mRNA avoidance, evasion or failure to trigger an immune response can be expressed in terms relative to any compound or substance which is known to trigger such a response.

The term “innate immune response” includes a cellular response to exogenous single stranded nucleic acids, generally of viral or bacterial origin, which involves the induction of cytokine expression and release, particularly the interferons, and cell death. As used herein, the innate immune response or interferon response operates at the single cell level causing cytokine expression, cytokine release, global inhibition of protein synthesis, global destruction of cellular RNA, upregulation of major histocompatibility molecules, and/or induction of apoptotic death, induction of gene transcription of genes involved in apoptosis, anti-growth, and innate and adaptive immune cell activation. Some of the genes induced by type I IFNs include PKR, ADAR (adenosine deaminase acting on RNA), OAS (2′,5′-oligoadenylate synthetase), RNase L, and Mx proteins. PKR and ADAR lead to inhibition of translation initiation and RNA editing, respectively. OAS is a dsRNA-dependent synthetase that activates the endoribonuclease RNase L to degrade ssRNA.

In some embodiments, the innate immune response comprises expression of a Type I or Type II interferon, and the expression of the Type I or Type II interferon is not increased more than two-fold compared to a reference from a cell which has not been contacted with an mRNA of the invention.

In some embodiments, the innate immune response comprises expression of one or more IFN signature genes and where the expression of the one of more IFN signature genes is not increased more than three-fold compared to a reference from a cell which has not been contacted with the mRNA of the invention.

While in some circumstances, it might be advantageous to eliminate the innate immune response in a cell, the invention provides mRNA that upon administration result in a substantially reduced (significantly less) the immune response, including interferon signaling, without entirely eliminating such a response.

In some embodiments, the immune response is lower by 10%, 20%, 30%, 40%, 50%, 60%, 70%, 80%, 90%, 95%, 99%, 99.9%, or greater than 99.9% as compared to the immune response induced by a reference compound. The immune response itself may be measured by determining the expression or activity level of Type 1 interferons or the expression of interferon-regulated genes such as the toll-like receptors (e.g., TLR7 and TLR8). Reduction of innate immune response can also be measured by measuring the level of decreased cell death following one or more administrations to a cell population; e.g., cell death is 10%, 25%, 50%, 75%, 85%, 90%, 95%, or over 95% less than the cell death frequency observed with a reference compound. Moreover, cell death may affect fewer than 50%, 40%, 30%, 20%, 10%, 5%, 1%, 0.1%, 0.01% or fewer than 0.01% of cells contacted with the mRNA.

In another embodiment, the mRNA of the present invention is significantly less immunogenic than an unmodified in vitro-synthesized RNA molecule polynucleotide, or primary construct with the same sequence or a reference compound. As used herein, “significantly less immunogenic” refers to a detectable decrease in immunogenicity. In another embodiment, the term refers to a fold decrease in immunogenicity. In another embodiment, the term refers to a decrease such that an effective amount of the mRNA can be administered without triggering a detectable immune response. In another embodiment, the term refers to a decrease such that the mRNA can be repeatedly administered without eliciting an immune response sufficient to detectably reduce expression of the recombinant protein. In another embodiment, the decrease is such that the mRNA can be repeatedly administered without eliciting an immune response sufficient to eliminate detectable expression of the recombinant protein.

In another embodiment, the mRNA is 2-fold less immunogenic than its unmodified counterpart or reference compound. In another embodiment, immunogenicity is reduced by a 3-fold factor. In another embodiment, immunogenicity is reduced by a 5-fold factor. In another embodiment, immunogenicity is reduced by a 7-fold factor. In another embodiment, immunogenicity is reduced by a 10-fold factor. In another embodiment, immunogenicity is reduced by a 15-fold factor. In another embodiment, immunogenicity is reduced by a fold factor. In another embodiment, immunogenicity is reduced by a 50-fold factor. In another embodiment, immunogenicity is reduced by a 100-fold factor. In another embodiment, immunogenicity is reduced by a 200-fold factor. In another embodiment, immunogenicity is reduced by a 500-fold factor. In another embodiment, immunogenicity is reduced by a 1000-fold factor. In another embodiment, immunogenicity is reduced by a 2000-fold factor. In another embodiment, immunogenicity is reduced by another fold difference.

Methods of determining immunogenicity are well known in the art, and include, e.g. measuring secretion of cytokines (e.g. IL-12, IFNalpha, TNF-alpha, RANTES, MIP-1alpha or beta, IL-6, IFN-beta, or IL-8), measuring expression of DC activation markers (e.g. CD83, HLA-DR, CD80 and CD86), or measuring ability to act as an adjuvant for an adaptive immune response.

The mRNA of the invention, including the combination of modifications taught herein may have superior properties making them more suitable as therapeutic modalities.

It has been determined that the “all or none” model in the art is sorely insufficient to describe the biological phenomena associated with the therapeutic utility of modified mRNA. The present inventors have determined that to improve protein production, one may consider the nature of the modification, or combination of modifications, the percent modification and survey more than one cytokine or metric to determine the efficacy and risk profile of a particular modified mRNA.

In one aspect of the invention, methods of determining the effectiveness of a modified mRNA as compared to unmodified involves the measure and analysis of one or more cytokines whose expression is triggered by the administration of the exogenous nucleic acid of the invention. These values are compared to administration of an unmodified nucleic acid or to a standard metric such as cytokine response, PolyIC, R-848 or other standard known in the art.

One example of a standard metric developed herein is the measure of the ratio of the level or amount of encoded polypeptide (protein) produced in the cell, tissue or organism to the level or amount of one or more (or a panel) of cytokines whose expression is triggered in the cell, tissue or organism as a result of administration or contact with the modified nucleic acid. Such ratios are referred to herein as the Protein:Cytokine Ratio or “PC” Ratio. The higher the PC ratio, the more efficacioius the modified nucleic acid (polynucleotide encoding the protein measured). Preferred PC Ratios, by cytokine, of the present invention may be greater than 1, greater than 10, greater than 100, greater than 1000, greater than 10,000 or more. Modified nucleic acids having higher PC Ratios than a modified nucleic acid of a different or unmodified construct are preferred.

The PC ratio may be further qualified by the percent modification present in the polynucleotide. For example, normalized to a 100% modified nucleic acid, the protein production as a function of cytokine (or risk) or cytokine profile can be determined.

In one embodiment, the present invention provides a method for determining, across chemistries, cytokines or percent modification, the relative efficacy of any particular modified the mRNA by comparing the PC Ratio of the modified nucleic acid (mRNA).

mRNA containing varying levels of nucleobase substitutions could be produced that maintain increased protein production and decreased immunostimulatory potential. The relative percentage of any modified nucleotide to its naturally occurring nucleotide counterpart can be varied during the IVT reaction (for instance, 100, 50, 25, 10, 5, 2.5, 1, 0.1, 0.01% 5 methyl cytidine usage versus cytidine; 100, 50, 25, 10, 5, 2.5, 1, 0.1, 0.01% pseudouridine or N1-methyl-pseudouridine usage versus uridine). mRNA can also be made that utilize different ratios using 2 or more different nucleotides to the same base (for instance, different ratios of pseudouridine and N1-methyl-pseudouridine). mRNA can also be made with mixed ratios at more than 1 “base” position, such as ratios of 5 methyl cytidine/cytidine and pseudouridine/N1-methyl-pseudouridine/uridine at the same time. Use of modified mRNA with altered ratios of modified nucleotides can be beneficial in reducing potential exposure to chemically modified nucleotides. Lastly, positional introduction of modified nucleotides into the mRNA which modulate either protein production or immunostimulatory potential or both is also possible. The ability of such mRNA to demonstrate these improved properties can be assessed in vitro (using assays such as the PBMC assay described herein), and can also be assessed in vivo through measurement of both mRNA-encoded protein production and mediators of innate immune recognition such as cytokines

In another embodiment, the relative immunogenicity of the mRNA and its unmodified counterpart are determined by determining the quantity of the mRNA required to elicit one of the above responses to the same degree as a given quantity of the unmodified nucleotide or reference compound. For example, if twice as much mRNA is required to elicit the same response, than the mRNA is two-fold less immunogenic than the unmodified nucleotide or the reference compound.

In another embodiment, the relative immunogenicity of the mRNA and its unmodified counterpart are determined by determining the quantity of cytokine (e.g. IL-12, IFNalpha, TNF-alpha, RANTES, MIP-1alpha or beta, IL-6, IFN-beta, or IL-8) secreted in response to administration of the mRNA, relative to the same quantity of the unmodified nucleotide or reference compound. For example, if one-half as much cytokine is secreted, than the mRNA is two-fold less immunogenic than the unmodified nucleotide. In another embodiment, background levels of stimulation are subtracted before calculating the immunogenicity in the above methods.

Provided herein are also methods for performing the titration, reduction or elimination of the immune response in a cell or a population of cells. In some embodiments, the cell is contacted with varied doses of the same mRNA and dose response is evaluated. In some embodiments, a cell is contacted with a number of different mRNA at the same or different doses to determine the optimal composition for producing the desired effect. Regarding the immune response, the desired effect may be to avoid, evade or reduce the immune response of the cell. The desired effect may also be to alter the efficiency of protein production.

The mRNA of the present invention may be used to reduce the immune response using the method described in International Publication No. WO2013003475, herein incorporated by reference in its entirety.

Additionally, certain modified nucleosides, or combinations thereof, when introduced into the mRNA of the invention will activate the innate immune response. Such activating molecules are useful as adjuvants when combined with polypeptides and/or other vaccines. In certain embodiments, the activating molecules contain a translatable region which encodes for a polypeptide sequence useful as a vaccine, thus providing the ability to be a self-adjuvant.

In one embodiment, the mRNA of the invention may encode an immunogen. The delivery of the mRNA encoding an immunogen may activate the immune response. As a non-limiting example, the mRNA encoding an immunogen may be delivered to cells to trigger multiple innate response pathways (see International Pub. No. WO2012006377: herein incorporated by reference in its entirety). As another non-limiting example, the mRNA of the present invention encoding an immunogen may be delivered to a vertebrate in a dose amount large enough to be immunogenic to the vertebrate (see International Pub. No. WO2012006372 and WO2012006369; each of which is herein incorporated by reference in their entirety). In some embodiments, the mRNA encodes an immunogen including, without limitation. Zika virus envelope protein (Env) antigens, KRAS antigens including one or more mutations associated with cancer, influenza virus antigens, cytomegalovirus (CMV) antigens (including gH, gL, UL128, UL130, UL131A, and herpesvirus glycoprotein (gB)), human metapneumovirus (HMPV) antigens, parainfluenza virus (PIV3) antigens, and cancer-associated neoepitopes.

The mRNA of invention may encode a polypeptide sequence for a vaccine and may further comprise an inhibitor. The inhibitor may impair antigen presentation and/or inhibit various pathways known in the art. As a non-limiting example, the mRNA of the invention may be used for a vaccine in combination with an inhibitor which can impair antigen presentation (see International Pub. No. WO2012089225 and WO2012089338: each of which is herein incorporated by reference in their entirety).

In one embodiment, the mRNA of the invention may be self-replicating RNA. Self-replicating RNA molecules can enhance efficiency of RNA delivery and expression of the enclosed gene product. In one embodiment, the mRNA may comprise at least one modification described herein and/or known in the art. In one embodiment, the self-replicating RNA can be designed so that the self-replicating RNA does not induce production of infectious viral particles. As a non-limiting example the self-replicating RNA may be designed by the methods described in US Pub. No. US20110300205 and International Pub. No. WO2011005799, each of which is herein incorporated by reference in their entirety.

In one embodiment, the self-replicating mRNA of the invention may encode a protein which may raise the immune response. As a non-limiting example, the mRNA may be self-replicating mRNA may encode at least one antigen (see US Pub. No. US20110300205 and International Pub. Nos. WO2011005799, WO2013006838 and WO2013006842: each of which is herein incorporated by reference in their entirety).

In one embodiment, the self-replicating mRNA of the invention may be formulated using methods described herein or known in the art. As a non-limiting example, the self-replicating RNA may be formulated for delivery by the methods described in Geall et al (Nonviral delivery of self-amplifying RNA vaccines, PNAS 2012; PMID: 22908294).

In one embodiment, the mRNA of the present invention may encode amphipathic and/or immunogenic amphipathic peptides.

In on embodiment, a formulation of the mRNA of the present invention may further comprise an amphipathic and/or immunogenic amphipathic peptide. As a non-limiting example, the mRNA comprising an amphipathic and/or immunogenic amphipathic peptide may be formulated as described in US. Pub. No. US20110250237 and International Pub. Nos. WO2010009277 and WO2010009065; each of which is herein incorporated by reference in their entirety.

In one embodiment, the mRNA of the present invention may be immunostimultory. As a non-limiting example, the mRNA may encode all or a part of a positive-sense or a negative-sense stranded RNA virus genome (see International Pub No. WO2012092569 and US Pub No. US20120177701, each of which is herein incorporated by reference in their entirety). In another non-limiting example, the immunostimultory mRNA of the present invention may be formulated with an excipient for administration as described herein and/or known in the art (see International Pub No. WO2012068295 and US Pub No. US20120213812, each of which is herein incorporated by reference in their entirety).

In one embodiment, the response of the vaccine formulated by the methods described herein may be enhanced by the addition of various compounds to induce the therapeutic effect. As a non-limiting example, the vaccine formulation may include a MHC II binding peptide or a peptide having a similar sequence to a MHC II binding peptide (see International Pub Nos. WO2012027365, WO2011031298 and US Pub No. US20120070493, US20110110965, each of which is herein incorporated by reference in their entirety). As another example, the vaccine formulations may comprise modified nicotinic compounds which may generate an antibody response to nicotine residue in a subject (see International Pub No. WO2012061717 and US Pub No. US20120114677, each of which is herein incorporated by reference in their entirety).

Naturally Occurring Mutants

In another embodiment, the mRNA can be utilized to express variants of naturally occurring proteins that have an improved disease modifying activity, including increased biological activity, improved patient outcomes, or a protective function, etc. Many such modifier genes have been described in mammals (Nadeau, Current Opinion in Genetics & Development 2003 13:290-295; Hamilton and Yu, PLoS Genet. 2012; 8:e1002644; Corder et al., Nature Genetics 1994 7:180-184; all herein incorporated by reference in their entireties). Examples in humans include Apo E2 protein, Apo A-I variant proteins (Apo A-1 Milano, Apo A-1 Paris), hyperactive Factor IX protein (Factor IX Padua Arg338Lys), transthyretin mutants (TTR Thr119Met). Expression of ApoE2 (cys112, cys158) has been shown to confer protection relative to other ApoE isoforms (ApoE3 (cys112, arg158), and ApoE4 (arg112, arg158)) by reducing susceptibility to Alzheimer's disease and possibly other conditions such as cardiovascular disease (Corder et al., Nature Genetics 1994 7:180-184; Seripa et al., Rejuvenation Res. 2011 14:491-500: Liu et al. Nat Rev Neurol. 2013 9:106-118; all herein incorporated by reference in their entireties). Expression of Apo A-I variants has been associated with reduced cholesterol (deGoma and Rader, 2011 Nature Rev Cardiol 8:266-271; Nissen et al., 2003 JAMA 290:2292-2300: all herein incorporated by reference in its entirety). The amino acid sequence of ApoA-I in certain populations has been changed to cysteine in Apo A-I Milano (Arg 173 changed to Cys) and in Apo A-I Paris (Arg 151 changed to Cys). Factor IX mutation at position R338L (FIX Padua) results in a Factor IX protein that has .about.10-fold increased activity (Simioni et al., N Engl J. Med. 2009 361:1671-1675: Finn et al., Blood. 2012 120:4521-4523; Cantore et al., Blood. 2012 120:4517-20; all herein incorporated by reference in their entireties). Mutation of transthyretin at positions 104 or 119 (Arg104 His, Thr119Met) has been shown to provide protection to patients also harboring the disease causing Val30Met mutations (Saraiva, Hum Mutat. 2001 17:493-503; DATA BASE ON TRANSTHYRETIN MUTATIONS www.ibmc.up.pt/mjsaraiva/ttrmut.html; all herein incorporated by reference in its entirety). Differences in clinical presentation and severity of symptoms among Portuguese and Japanese Met 30 patients carrying respectively the Met 119 and the His104 mutations are observed with a clear protective effect exerted by the non pathogenic mutant (Coelho et al. 1996 Neuromuscular Disorders (Suppl) 6: S20; Terazaki et al. 1999. Biochem Biophys Res Commun 264: 365-370; all herein incorporated by reference in its entirety), which confer more stability to the molecule. A modified mRNA encoding these protective TTR alleles can be expressed in TTR amyloidosis patients, thereby reducing the effect of the pathogenic mutant TTR protein.

As described herein, the phrase “major groove interacting partner” refers to RNA recognition receptors that detect and respond to RNA ligands through interactions, e.g. binding, with the major groove face of a nucleotide or nucleic acid. As such, RNA ligands comprising modified nucleotides or nucleic acids such as the mRNA as described herein decrease interactions with major groove binding partners, and therefore decrease an innate immune response.

Example major groove interacting, e.g. binding, partners include, but are not limited to the following nucleases and helicases. Within membranes, TLRs (Toll-like Receptors) 3, 7, and 8 can respond to single- and double-stranded RNAs. Within the cytoplasm, members of the superfamily 2 class of DEX(D/H) helicases and ATPases can sense RNAs to initiate antiviral responses. These helicases include the RIG-I (retinoic acid-inducible gene I) and MDA5 (melanoma differentiation-associated gene 5). Other examples include laboratory of genetics and physiology 2 (LGP2), HIN-200 domain containing proteins, or Helicase-domain containing proteins.

In some embodiments, an mRNA contained in an mRNA delivery complex according to any of the embodiments described herein encodes a tumor suppressor protein, wherein the protein corresponds to a tumor suppressor gene. In some embodiments, the tumor-suppressor protein is a Retinoblastoma protein (pRb). In some embodiments, the tumor-suppressor protein is a p53 tumor-suppressor protein. In some embodiments, the corresponding tumor-suppressor gene is Phosphatase and tensin homolog (PTEN). In some embodiments, the corresponding tumor-suppressor gene is BRCA1. In some embodiments, the corresponding tumor-suppressor gene is BRCA2. In some embodiments, the corresponding tumor-suppressor gene is Retinoblastoma RB (or RB1). In some embodiments, the corresponding tumor-suppressor gene is TSC1. In some embodiments, the corresponding tumor-suppressor gene is TSC2. In some embodiments, the corresponding tumor-suppressor gene includes, without limitation, Retinoblastoma RB (or RB1), TP53, TP63, TP73, CDKN2A (INK4A), CDKN1B, CDKN1C, DLD/NP1, HEPACAM, SDHB, SDHD, SFRP1, TCF21, TIG1, MLH1, MSH2, MSH6, WT1, WT2, NF1, NF2N, VHL, KLF4, pVHL, APC, CD95, ST5, YPEL3, ST7, APC, MADR2, BRCA1, BRCA2, Patched, TSC1, TSC2, PALB2, ST14, or VHL.

In some embodiments, the mRNA encodes a tumor suppressor protein PTEN. In some embodiments, the tumor suppressor protein PTEN is encoded by a human PTEN sequence. In some embodiments, the mRNA comprises a sequence selected from the group consisting of sequences with accession number of BC005821, JF268690, U92436, CR450306, AK024986, AK313581, U96180, and U93051 and NM_000314 in NCBI GenBank.

In some embodiments, the mRNA encodes a tumor suppressor protein p53. In some embodiments, the tumor suppressor protein p53 is encoded by a human TP53 sequence. In some embodiments, the mRNA comprises a sequence selected from the group consisting of sequences with accession number of AF052180, NM_000546, AY429684, BT019622, AK223026, DQ186652, DQ186651, DQ186650, DQ186649, DQ186648, DQ263704, DQ286964, DQ191317, DQ401704, AF307851, AM076972, AM076971, AM076970, DQ485152, BC003596, DQ648887, DQ648886, DQ648885, DQ648884, AK225838, M14694, M14695, EF101869. EF101868, EF101867, X01405, AK312568, NM_001126117, NM_001126116, NM_001126115, NM_001126114, NM_001126113, NM_001126112, FJ207420, X60020, X60019, X60018, X60017, X60016, X60015, X60014, X60013, X60011, X60012, X60010, X02469, S66666, AB082923, NM_001126118, JN900492, NM_001276699, NM_001276698, NM_001276697, NM 001276761, NM_001276760, NM_001276696, and NM_001276695 in NCBI GenBank.

In some embodiments, the mRNA encodes a tumor suppressor protein BRCA1. In some embodiments, the tumor suppressor protein BRCA1 is encoded by a human BRCA1 sequence. In some embodiments, the mRNA comprises a sequence selected from the group consisting of a sequence with accession number of NM_007294, NM_007297, NM_007298, NM_007304, NM_007299, NM_007300, BC046142, BC062429, BC072418, AY354539, AY751490, BC085615, BC106746, BC106745, BC114511, BC115037, U14680, AK293762, U68041, BC030969, BC012577, AK316200, DQ363751, DQ333387, DQ333386, Y08864, JN686490, AB621825, BC038947, U64805, and AF005068 in NCBI GenBank.

In some embodiments, the mRNA encodes a tumor suppressor protein BRCA2. In some embodiments, the tumor suppressor protein BRCA2 is encoded by a human BRCA2 sequence. In some embodiments, the mRNA comprises a sequence selected from the group consisting of a sequence with accession number of BC047568, NM_000059, DQ897648, BC026160 in NCBI GenBank.

In some embodiments, the mRNA encodes a tumor suppressor protein TSC1. In some embodiments, the tumor suppressor protein TSC1 is encoded by a human TSC1 sequence. In some embodiments, the mRNA comprises a sequence selected from the group consisting of a sequence with accession number of BC047772, NM_000368, BC070032, AB190910, BC108668, BC121000, NM_001162427, NM_001162426. D87683, and AF013168 in NCBI GenBank.

In some embodiments, the mRNA encodes a tumor suppressor protein TSC2. In some embodiments, the tumor suppressor protein TSC2 is encoded by a human TSC2 sequence. In some embodiments, the mRNA comprises a sequence selected from the group consisting of a sequence with accession number of BC046929, BX647816, AK125096, NM_000548, AB210000, NM_001077183, BC150300, BC025364, NM_001114382, AK094152, AK299343, AK295728, AK295672, AK294548, and X75621 in NCBI GenBank.

In some embodiments, the mRNA encodes a tumor suppressor protein Retinoblastoma 1 (RB1). In some embodiments, the tumor suppressor protein RB1 is encoded by a human RB1 sequence. In some embodiments, the mRNA comprises a sequence selected from the group consisting of a sequence with accession number of NM_000321, AY429568, AB208788, M19701, AK291258, L41870, AK307730, AK307125, AK300284, AK299179, M33647, M15400, M28419, BC039060, BC040540, and AF043224 in NCBI GenBank.

In some embodiments, an mRNA contained in an mRNA delivery complex according to any of the embodiments described herein encodes a protein, wherein the deficiency of the protein results in a disease or disorder. In some embodiments, the protein is Frataxin. In some embodiments, the protein is alpha 1 antitrypsin. In some embodiments, the protein is factor VIII. In some embodiments, the protein is factor IX.

In some embodiments, an mRNA contained in an mRNA delivery complex according to any of the embodiments described herein encodes a protein, wherein expression of the protein in an individual modulates an immune response to the protein in the individual. In some embodiments, the protein is an antigen. In some embodiments, the antigen is a disease-associated antigen (e.g., a tumor-associated antigen), and expression of the antigen in the individual results in an increased immune response to the antigen in the individual. In some embodiments, the antigen is a self-antigen, and expression of the antigen in the individual results in a decreased immune response to the antigen in the individual.

In some embodiments, an mRNA contained in an mRNA delivery complex according to any of the embodiments described herein encodes an antibody or antigen-binding fragment thereof. In some embodiments, the antibody is a therapeutic antibody. In some embodiments, the antibody is a bispecific antibody, such as a bispecific T cell engager (BiTE). In some embodiments, the antibody specifically binds to a disease-associated antigen, such as a tumor-associated antigen.

In some embodiments, an mRNA contained in an mRNA delivery complex according to any of the embodiments described herein comprises a reporter mRNA. In some embodiments, the mRNA comprises an EGFP mRNA, for example, CleanCap EGFP mRNA, CleanCap EGFP mRNA (5 moU), or CleanCap Cyanine 5 EGFP mRNA (5 moU). In some embodiments, the mRNA comprises a Luc mRNA, for example, CleanCap Fluc mRNA, CleanCap Fluc mRNA (5 moU), CleanCap Cyanine 5 Fluc mRNA (5 moU), CleanCap Gaussia Luc mRNA (5 moU), or CleanCap Renilla Luc mRNA (5 moU). In some embodiments, the mRNA comprises an mRNA selected from CleanCap β-gal mRNA, CleanCap β-gal mRNA (5 moU) and CleanCap mCherry mRNA (5 moU).

In some embodiments, an mRNA delivery complex according to any of the embodiments described herein further comprises an interfering RNA (RNAi), or is to be used in combination with an RNAi. In some embodiments, the RNAi includes, without limitation, an siRNA, shRNA, or miRNA. In some embodiments, the RNAi is an siRNA. In some embodiments, the RNAi is a microRNA. In some embodiments, the RNAi targets an endogenous gene. In some embodiments, the RNAi targets an exogenous gene. In some embodiments, the RNAi targets a disease-associated gene, e.g., a cancer-associated genes, such as an oncogene. In some embodiments, the RNAi targets an oncogene. In some embodiments, the oncogene is Smoothened. In some embodiments, the oncogene is rasK. In some embodiments, the oncogene is KRAS.

In some embodiments, the RNAi (e.g., siRNA) targets an oncogene, wherein the oncogene is KRAS. In some embodiments, the individual comprises an aberration of KRAS. In some embodiments, the aberration of KRAS comprises a mutation on codon 12, 13, 17, 34 or 61 of KRAS. In some embodiments, an aberration of KRAS is selected from the group consisting of G12C, G12S, G12R, G12F, G12L, G12N, G12A, G12D, G12S, G12V, G13C, G13S, G13R, G13A, G13D, G13V, G13P, S17G, P34S, Q61E, Q61K, Q61L, Q61R, Q61P, Q61H, K117N, A146P, A146T and A146V. In some embodiments, the aberration of KRAS is selected from the group consisting of G12C, G12S, G12R, G12F, G12L, G2N, G12A, G12D, G2V, G13C, G13S, G13D, G13V, G13P, S17G, P34S, Q61K, Q61L, Q61R, and Q61H. In some embodiments, the aberration of KRAS is selected from the group consisting of G12C, G12R, G12S, G12A, G12D, G12V, G13C, G13R, G13S, G13A, G13D, G13V, Q61K, Q61L, Q61R, Q61H, K117N, A146P, A146T and A146V. In some embodiments, the aberration of KRAS is selected from the group consisting of KRAS G12A, G12C, G12D, G12R, G12S, G12V, G13A, G13C, G13D, G13R, G13S, G13V, Q61E, Q61H, Q61K, Q61L, Q61P, and Q61R. In some embodiments, the aberration of KRAS comprises G12C. In some embodiments, the aberration of KRAS comprises G12D. In some embodiments, the aberration of KRAS comprises Q61K. In some embodiments, the aberration of KRAS comprises G12C and G12D. In some embodiments, the aberration of KRAS comprises G12C and Q61K. In some embodiments, the aberration of KRAS comprises G12D and Q61K. In some embodiments, the aberration of KRAS comprises G12C, G12D and Q61K.

In some embodiments, the RNAi (e.g., siRNA) targets a mutant form of KRAS. In some embodiments, the RNAi (e.g., siRNA) specifically targets a mutant form of KRAS but not the wildtype form of KRAS. In some embodiments, the mutant form comprises an aberration of KRAS, wherein the aberration of KRAS comprises a mutation on codon 12, 13, 17, 34 or 61 of KRAS. In some embodiments, the mutant form comprises an aberration of KRAS, wherein the aberration of KRAS is selected from the group consisting of G12C, G12S, G12R, G12F, G12L, G12N, G12A, G12D, G12S, G12V, G13C, G13S, G13R, G13A, G13D, G13V, G13P, S17G, P34S, Q61E, Q61K, Q61L, Q61R, Q61P, Q61H, K117N, A146P, A146T and A146V. In some embodiments, the mutant form comprises an aberration of KRAS, wherein the aberration of KRAS is selected from the group consisting of G12C, G12S, G12R, G12F, G12L, G12N, G12A, G12D, G12V, G13C, G13S, G13D, G13V, G13P, S17G, P34S, Q61K, Q61L, Q61R, and Q61H. In some embodiments, the mutant form comprises an aberration of KRAS, wherein the aberration of KRAS is selected from the group consisting of G12C, G12R, G12S, G12A, G12D, G12V, G13C, G13R, G13S, G13A, G13D, G13V, Q61K, Q61L, Q61R, Q61H, K117N, A146P, A146T and A146V. In some embodiments, the mutant form comprises an aberration of KRAS, wherein the aberration of KRAS is selected from the group consisting of KRAS G12A, G12C, G12D, G12R, G12S, G12V, G13A, G13C, G3D, G13R, G13S, G13V, Q61E, Q61H, Q61K, Q61L, Q61P. and Q61R. In some embodiments, the aberration of KRAS is selected from the group consisting of KRAS G12C, G12D, G12R, G12S, G12V and G13D. In some embodiments, the aberration of KRAS comprises G12C. In some embodiments, the aberration of KRAS comprises G12D. In some embodiments, the aberration of KRAS comprises Q61K. In some embodiments, the aberration of KRAS comprises G12C and G12D. In some embodiments, the aberration of KRAS comprises G12C and Q61K. In some embodiments, the aberration of KRAS comprises G12D and Q61K. In some embodiments, the aberration of KRAS comprises G12C, G12D and Q61K.

In some embodiments, the RNAi (e.g., siRNA) targets a plurality of mutant forms of KRAS. In some embodiments, the plurality of mutant forms comprises a plurality of aberrations of KRAS, wherein the plurality of aberrations of KRAS comprise at least two or more mutations on codon 12, 13, 17, 34 and/or 61 of KRAS. In some embodiments, the plurality of aberrations of KRAS comprises at least two or more mutations on codon 12 and 61 of KRAS. In some embodiments, the aberration of KRAS is selected from the group consisting of G12C, G2S, G12R, G12F, G12L, G12N, G12A, G12D, G12S, G12V, G13C, G13S, G13R, G13A, G13D, G13V, G13P, S17G, P34S, Q61E, Q61K, Q61L, Q61R Q61P, Q61H, K117N, A146P, A146T and A146V. In some embodiments, the aberrations of KRAS are selected from the group consisting of G12C, G12S, G12R, G12F, G12L, G12N, G12A, G12D, G12V, G13C, G13S, G13D, G13V, G13P, S17G, P34S, Q61K, Q61L, Q61R, and Q61H. In some embodiments, the aberrations of KRAS are selected from the group consisting of G12C, G12R, G12S, G12A, G12D, G12V, G13C, G13R, G13S, G13A, G13D, G13V, Q61K. Q61L, Q61R, Q61H, K117N, A146P, A146T and A146V. In some embodiments, the aberrations of KRAS is selected from the group consisting of KRAS G12A, G12C, G12D, G12R, G12S, G12V, G13A, G13C, G13D, G13R, G13S, G13V, Q61E, Q61H, Q61K, Q61L, Q61P, and Q61R. In some embodiments, the aberrations of KRAS are selected from the group consisting of KRAS G12C, G12D, G12R, G12S, G12V and G13D. In some embodiments, the aberrations of KRAS are selected from the group consisting of KRAS G12C, G12D, and Q61K. In some embodiments, the aberrations of KRAS comprise G12C and G12D. In some embodiments, the aberrations of KRAS comprise G12C and Q61K. In some embodiments, the aberrations of KRAS comprise G12D and Q61K. In some embodiments, the aberration of KRAS comprises G12C, G12D and Q61K.

In some embodiments, the RNAi (e.g., siRNA) comprises a plurality of RNAi (e.g., siRNA) comprising a first RNAi (e.g., a first siRNA) and a second RNAi (e.g., a second siRNA), wherein the first RNAi targets a first mutant form of KRAS, and wherein the second RNAi targets a second mutant form of KRAS. In some embodiments, the first RNAi and/or the second RNAi do not target the wildtype form of KRAS. In some embodiments, the first mutant form and/or the second mutant form comprises an aberration of KRAS, wherein the aberration of KRAS comprises a mutation on codon 12, 13, 17, 34 and/or 61 of KRAS. In some embodiments, the first mutant form and/or the second mutant form comprises an aberration of KRAS, wherein the aberration of KRAS comprises a mutation on codon 12 or 61 of KRAS. In some embodiments, the first mutant form comprises an aberration of KRAS comprising a mutation on codon 12, and the second mutant form comprises an aberration of KRAS comprising a mutation on codon 61. In some embodiments, the first mutant form and/or the second mutant form comprises an aberration of KRAS, wherein the aberration of KRAS is selected from the group consisting of G12C, G12S, G12R, G12F, G12L, G12N, G12A, G12D, G12S, G12V, G13C, G13S, G13R, G13A, G13D, G13V, G13P, S17G, P34S, Q61E, Q61K, Q61L, Q616R, Q61P, Q61H, K117N, A146P, A146T and A146V. In some embodiments, the first mutant form and/or the second mutant form comprises an aberration of KRAS, wherein the aberration of KRAS is selected from the group consisting of G12C, G12S, G12R, G12F, G12L, G12N, G12A, G12D, G12V, G13C, G13S, G13D, G13V, G13P, S17G, P34S, Q61K, Q61L, Q61R, and Q61H. In some embodiments, the first mutant form and/or the second mutant form comprises an aberration of KRAS, wherein the aberration of KRAS is selected from the group consisting of G12C, G12R, G12S, G12A, G12D, G12V, G13C, G13R, G13S, G13A, G13D, G13V, Q61K, Q61L, Q61R, Q61H, K117N, A146P, A146T and A146V. In some embodiments, the first mutant form and/or the second mutant form comprises an aberration of KRAS, wherein the aberration of KRAS is selected from the group consisting of KRAS G12A, G12C, G12D, G12R, G12S, G12V, G13A, G13C, G13D, G13R, G13S, G13V, Q61E, Q61H, Q61K, Q61L, Q61P and Q61R. In some embodiments, the first mutant form and/or the second mutant form comprises an aberration of KRAS, wherein the aberration of KRAS is selected from the group consisting of KRAS G12C, G12D, G12R, G12S, G12V and G13D. In some embodiments, the first mutant form and/or the second mutant form comprises an aberration of KRAS, wherein the aberration of KRAS is selected from G12C, G12D and Q61K. In some embodiments, the first mutant form comprises an aberration of KRAS comprising KRAS G12C, and the second mutant form comprises an aberration of KRAS comprising KRAS G12D. In some embodiments, the first mutant form comprises an aberration of KRAS comprising KRAS G12C, and the second mutant form comprises an aberration of KRAS comprising KRAS Q61K. In some embodiments, the first mutant form comprises an aberration of KRAS comprising KRAS G12D, and the second mutant form comprises an aberration of KRAS comprising KRAS Q61K.

In some embodiments, the RNAi (e.g., siRNA) comprises a plurality of RNAi (e.g., siRNA) comprising a first RNAi (e.g., a first siRNA), a second RNAi (e.g., a second siRNA), and a third RNAi (e.g., siRNA). In some embodiments, the first RNAi targets a first mutant form of KRAS, the second RNAi targets a second mutant form of KRAS, and the third RNAi targets a third mutant form of KRAS. In some embodiments, the first, second and third KRAS mutant form each comprises an aberration of KRAS comprising a mutation on codon 12, 13, 17, 34 and/or 61 of KRAS. In some embodiments, the first, second and third KRAS mutant form each comprises an aberration of KRAS selected from the group consisting of G12C, G12S, G12R, G12F, G12L, G12N, G12A, G12D, G12S, G12V, G13C, G13S, G13R, G13A, G13D, G13V, G13P, S17G, P34S, Q61E, Q61K, Q61L, Q61R, Q61P, Q61H, K117N, A146P, A146T and A146V. In some embodiments, the first, second and third KRAS mutant form each comprises an aberration of KRAS selected from the group consisting of G12C, G12S, G12R, G12F, G12L, G12N, G12A, G12D, G12V, G13C, G13S, G13D, G13V, G13P, S17G, P34S, Q61K, Q61L, Q61R, and Q61H. In some embodiments, the first, second and third KRAS mutant form each comprises an aberration of KRAS selected from the group consisting of G12C, G12R, G12S, G12A, G12D, G12V, G13C, G13R, G13S, G13A, G13D, G13V, Q61K. Q61L, Q61R, Q61H, K117N, A146P, A146T and A146V. In some embodiments, the first, second and third KRAS mutant form each comprises an aberration of KRAS selected from the group consisting of KRAS G12A, G12C, G12D, G12R, G12S, G12V, G13A, G13C, G13D, G13R, G13S, G13V, Q61E, Q61H, Q61K, Q61L, Q61P, and Q61R. In some embodiments, the first, second and third KRAS mutant form each comprises an aberration of KRAS selected from the group consisting of KRAS G12C, G12D, G12R, G12S, G12V, G13D and Q61K. In some embodiments, the first, second and third KRAS mutant form each comprises an aberration of KRAS selected from the group consisting of G12C, G12D and Q61K. In some embodiments, the first mutant form comprises an aberration of KRAS comprising KRAS G12C, the second mutant form comprises an aberration of KRAS comprising KRAS G12D, and the third mutant form comprises an aberration of KRAS comprising KRAS Q61K.

In some embodiments, the RNAi (e.g., siRNA) comprises an RNAi (e.g., siRNA) targeting KRAS comprising a sequence of 5′-GUUGGAGCUUGUGGCGUAGTT-3′ (sense) (SEQ ID NO: 83), 5′-CUACGCCACCAGCUCCAACTT-3 (anti-sense) (SEQ ID NO: 84), 5′-GAAGUGCAUACACCGAGACTT-3′ (sense) (SEQ ID NO: 86), 5′-GUCUCGGUGUAGCACUUCTT-3′ (anti-sense) (SEQ ID NO: 87), 5′-GUUGGAGCUGUUGGCGUAGTT-3′ (sense) (SEQ ID NO: 88) and/or 5′-CUACGCCAACAGCUCCAACTT-3′ (anti-sense) (SEQ ID NO: 89). In some embodiments, the RNAi (e.g., siRNA) comprises an RNAi (e.g., siRNA) targeting KRAS comprising a nucleic acid sequence selected from sequences with SEQ ID NOS: 83, 84, 86-89. In some embodiments, the RNAi (e.g., siRNA) comprises an RNAi (e.g., siRNA) targeting KRAS comprising a sequence targeting KRAS G12S, such as the siRNA sequences disclosed in Acunzo, M. et al., Proc Nat Acad Sci USA. 2017 May 23:114(21):E4203-E4212. In some embodiments, the RNAi (e.g., siRNA) comprises an RNAi (e.g., siRNA) targeting KRAS as disclosed in WO2014013995, JP2013212052, WO2014118817, WO2012129352, WO2017179660, JP2013544505, U.S. Pat. Nos. 8,008,474, 7,745,611, 7,576,197, 7,507,811, each of which is incorporated fully in this application.

In some embodiments, the RNAi includes, without limitation, siRNA, shRNA, and miRNA. The term “interfering RNA” or “RNAi” or “interfering RNA sequence” refers to single-stranded RNA (e.g., mature miRNA) or double-stranded RNA (i.e., duplex RNA such as siRNA, aiRNA, or pre-miRNA) that is capable of reducing or inhibiting the expression of a target gene or sequence (e.g., by mediating the degradation or inhibiting the translation of mRNAs which are complementary to the interfering RNA sequence) when the interfering RNA is in the same cell as the target gene or sequence, interfering RNA thus refers to the single-stranded RNA that is complementary to a target mRNA sequence or to the double-stranded RNA formed by two complementary strands or by a single, self-complementary strand. Interfering RNA may have substantial or complete identity to the target gene or sequence, or may comprise a region of mismatch (i.e., a mismatch motif). The sequence of the interfering RNA can correspond to the full-length target gene, or a subsequence thereof. Interfering RNA includes “small-interfering RNA” or “siRNA,” e.g., interfering RNA of about 15-60, 15-50, or 5-40 (duplex) nucleotides in length, more typically about 15-30, 15-25, or 19-25 (duplex) nucleotides in length, and is preferably about 20-24, 21-22, or 21-23 (duplex) nucleotides in length (e.g., each complementary sequence of the double-stranded siRNA is 15-60, 15-50, 1540, 15-30, 15-25, or 19-25 nucleotides in length, preferably about 20-24, 21-22, or 21-23 nucleotides in length, and the double-stranded siRNA is about 15-60, 15-50, 15-40, 5-30, 5-25, or 19-25 base pairs in length, preferably about 8-22, 9-20, or 19-21 base pairs in length). siRNA duplexes may comprise 3′ overhangs of about 1 to about 4 nucleotides or about 2 to about 3 nucleotides and 5′ phosphate termini. Examples of siRNA include, without limitation, a double-stranded polynucleotide molecule assembled from two separate stranded molecules, wherein one strand is the sense strand and the other is the complementary antisense strand: a double-stranded polynucleotide molecule assembled from a single stranded molecule, where the sense and antisense regions are linked by a nucleic acid-based or non-nucleic acid-based linker; a double-stranded polynucleotide molecule with a hairpin secondary structure having self-complementary sense and antisense regions; and a circular single-stranded polynucleotide molecule with two or more loop structures and a stem having self-complementary sense and antisense regions, where the circular polynucleotide can be processed in vivo or in vitro to generate an active double-stranded siRNA molecule. Preferably, siRNA are chemically synthesized. siRNA can also be generated by cleavage of longer dsRNA (e.g., dsRNA greater than about 25 nucleotides in length) with the E coli RNase III or Dicer. These enzymes process the dsRNA into biologically active siRNA (see, e.g., Yang et al., Proc Natl. Acad. Set. USA, 99:9942-9947 (2002); Calegari et al., Proc. Natl. Acad. Sci. USA, 99: 14236 (2002); Byrom et al., Ambion TeehNotes, 10(1):4-6 (2003); Kawasaki et al., Nucleic Acids Res., 3 1:981-987 (2003); Knight et al., Science, 293:2269-2271 (2001); and Robertson et al., J. Biol. Chem., 243:82 (1968)). Preferably, dsRNA are at least 50 nucleotides to about 100, 200, 300, 400, or 500 nucleotides in length A dsRNA may be as long as 1000, 1500, 2000, 5000 nucleotides in length, or longer. The dsRNA can encode for an entire gene transcript or a partial gene transcript. In certain instances, siRNA may be encoded by a plasmid (e.g., transcribed as sequences that automatically fold into duplexes with hairpin loops). A small hairpin RNA or short hairpin RNA (shRNA) is a sequence of RNA that makes a tight hairpin turn that can be used to silence gene expression via RNA interference. The shRNA hairpin structure is cleaved by the cellular machinery into siRNA, which is then bound to the RNA-induced silencing complex (RISC). This complex binds to and cleaves mRNAs which match the siRNA that is bound to it. Suitable lengths of the RNAi include, without limitation, about 5 to about 200 nucleotides, or 10-50 nucleotides or base pairs or 15-30 nucleotides or base pairs. In some embodiments, the RNAi is substantially complementary (such as at least about 60%, 70%, 80%, 90%, 95%, 98%, 99%, or more identical to) the corresponding target gene. In some embodiments, the RNAi is modified, for example by incorporating non-naturally occurring nucleotides.

In some embodiments, the RNAi is a double-stranded RNAi. Suitable lengths of the RNAi include, without limitation, about 5 to about 200 nucleotides, or 10-50 nucleotides or base pairs or 15-30 nucleotides or base pairs. In some embodiments, the RNAi is substantially complementary (such as at least about 60%, 70%, 80%, 90%, 95%, 98%, 99%, or more identical) to the corresponding target gene. In some embodiments, the RNAi is modified, for example by incorporating non-naturally occurring nucleotides.

In some embodiments, the RNAi specifically targets an RNA molecule, such as an mRNA, encoding a protein involved in a disease, such as cancer. In some embodiments, the disease is cancer, such as a solid tumor or hematological malignancy, and the interfering RNA targets mRNA encoding a protein involved in the cancer, such as a protein involved in regulating the progression of the cancer. In some embodiments, the RNAi targets an oncogene involved in the cancer.

In some embodiments, the RNAi specifically targets an RNA molecule, such as an mRNA, encoding a protein involved in negatively regulating an immune response. In some embodiments, the interfering RNA targets mRNA encoding a negative co-stimulatory molecule. In some embodiments, the negative co-stimulatory molecule includes, for example, PD-1, PD-L1, PD-L2, TIM-3, BTLA, VISTA, LAG-3, and CTLA-4.

In some embodiments, the RNAi is an miRNA. A microRNA (abbreviated miRNA) is a short ribonucleic acid (RNA) molecule found in eukaryotic cells. A microRNA molecule has very few nucleotides (an average of 22) compared with other RNAs. miRNAs are post-transcriptional regulators that bind to complementary sequences on target messenger RNA transcripts (mRNAs), usually resulting in translational repression or target degradation and gene silencing. The human genome may encode over 1000 miRNAs, which may target about 60% of mammalia genes and are abundant in many human cell types. Suitable lengths of the miRNAs include, without limitation, about 5 to about 200 nucleotides, or about 0-50 nucleotides or base pairs or 15-30 nucleotides or base pairs. In some embodiments, the miRNA is substantially complementary (such as at least about 60%, 70%, 80%, 90%, 95%, 98%, 99%, or more identical to) the corresponding target gene. In some embodiments, the miRNA is modified, for example by incorporating non-naturally occurring nucleotides.

Modification of mRNA and/or RNAi

In some embodiments, any mRNA and/or RNAi molecules described herein are modified. Modified mRNA or RNAi have structural and/or chemical features that avoid one or more of the problems in the art, for example, features which are useful for optimizing nucleic acid-based therapeutics while retaining structural and functional integrity, overcoming the threshold of expression, improving expression rates, half life and/or protein concentrations, optimizing protein localization, and avoiding deleterious bio-responses such as the immune response and/or degradation pathways. Modifications of the mRNA and/or RNAi may be on the nucleoside base and/or sugar portion of the nucleosides which comprise the mRNA or RNAi.

Representative U.S. patents and patent applications that teach the some examples of the modified mRNA and/or RNAi molecules and the preparation thereof include, but are not limited to, U.S. Pat. No. 8,802,438, U.S. Pat. Appl. No. 2013/0123481, each of which is herein incorporated by reference in its entirety.

In some embodiments, mRNA and/or RNAi molecules are modified to improve the stability and/or clearance in tissues, receptor uptake and/or kinetics, cellular access by the compositions, engagement with translational machinery, half-life, translation efficiency, immune evasion, protein production capacity, secretion efficiency (when applicable), accessibility to circulation, protein half-life and/or modulation of a cell's status, function and/or activity.

The mRNA or RNAi can include any useful modification, such as to the sugar, the nucleobase, or the internucleoside linkage (e.g. to a linking phosphate/to a phosphodiester linkage/to the phosphodiester backbone). For example, the major groove of a mRNA or RNAi. or the major groove face of a nucleobase may comprise one or more modifications. One or more atoms of a pyrimidine nucleobase (e.g. on the major groove face) may be replaced or substituted with optionally substituted amino, optionally substituted thiol, optionally substituted alkyl (e.g., methyl or ethyl), or halo (e.g., chloro or fluoro). In certain embodiments, modifications (e.g., one or more modifications) are present in each of the sugar and the internucleoside linkage. Modifications according to the present invention may be modifications of ribonucleic acids (RNAs) to deoxyribonucleic acids (DNAs), e.g., the substitution of the 2′ OH of the ribofuranysyl ring to 2′ H, threose nucleic acids (TNAs), glycol nucleic acids (GNAs), peptide nucleic acids (PNAs), locked nucleic acids (LNAs) or hybrids thereof). Additional modifications are described herein.

In some embodiments, the modification is on the nucleobase and is selected from the group consisting of pseudouridine or N1-methylpseudouridine. In some embodiments, the modified nucleoside is not pseudouridine (ψ) or 5-methyl-cytidine (m5C).

In some embodiments, multiple modifications are included in the modified nucleic acid or in one or more individual nucleoside or nucleotide of the mRNA or RNAi. For example, modifications to a nucleoside may include one or more modifications to the nucleobase and the sugar.

In some embodiments, the mRNA and/or RNAi are chemically modified on the major groove face, thereby disrupting major groove binding partner interactions, which may cause innate immune responses.

In some embodiments, the mRNA and/or RNAi molecules comprise a nucleotide that disrupts binding of a major groove interacting. e.g. binding, partner with a nucleic acid, wherein the nucleotide has decreased binding affinity to major groove interacting partners.

In some embodiments, the mRNA and/or RNAi molecules comprise nucleotides that contain chemical modifications, wherein the nucleotide has altered binding to major groove interacting partners. In some embodiments, the chemical modifications are located on the major groove face of the nucleobase, and wherein the chemical modifications can include replacing or substituting an atom of a pyrimidine nucleobase with an amine, an SH, an alkyl (e.g., methyl or ethyl), or a halo (e.g., chloro or fluoro). In some embodiments, the chemical modification is located on the sugar moiety of the nucleotide. In some embodiments, the chemical modification is located on the phosphate backbone of the nucleic acid. In some embodiments, the chemical modifications alter the electrochemistry on the major groove face of the nucleic acid.

In some embodiments, the mRNA and/or RNAi molecules comprise a nucleotide that contain chemical modifications, wherein the nucleotide reduces the cellular innate immune response, as compared to the cellular innate immune induced by a corresponding unmodified nucleic acid.

The modifications may be various distinct modifications. In some embodiments, the mRNA is modified, wherein the coding region, the flanking regions and/or the terminal regions may contain one, two, or more (optionally different) nucleoside or nucleotide modifications.

In some embodiments, modified mRNA and/or RNAi introduced to a cell may exhibit reduced degradation and/or reduced cell's innate immune or interferon response, as compared to an unmodified polynucleotide. RNA. Modifications include, but are not limited to, for example, (a) end modifications, e.g., 5′ end modifications (phosphorylation dephosphorylation, conjugation, inverted linkages, etc.), 3′ end modifications (conjugation, DNA nucleotides, inverted linkages, etc.), (b) base modifications, e.g., replacement with modified bases, stabilizing bases, destabilizing bases, or bases that base pair with an expanded repertoire of partners, or conjugated bases, (c) sugar modifications (e.g., at the 2′ position or 4′ position) or replacement of the sugar, as well as (d) internucleoside linkage modifications, including modification or replacement of the phosphodiester linkages. To the extent that such modifications interfere with translation of an mRNA (i.e., results in a reduction of 50% or more in translation relative to the lack of the modification—e.g., in a rabbit reticulocyte in vitro translation assay), the modification is not suitable for the methods and compositions described herein. Specific examples of modified mRNA or RNAi molecule useful with the methods described herein include, but are not limited to, RNA molecules containing modified or non-natural internucleoside linkages. Modified mRNA or RNAi molecule having modified internucleoside linkages includes, among others, those that do not have a phosphorus atom in the internucleoside linkage. In other embodiments, the synthetic, modified RNA has a phosphorus atom in its internucleoside linkage(s).

Non-limiting examples of modified internucleoside linkages include phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those) having inverted polarity wherein the adjacent pairs of nucleoside units are linked 3′-5′ to 5′-3′ or 2′-5′ to 5′-2′. Various salts, mixed salts and free acid forms are also included.

Modified internucleoside linkages that do not include a phosphorus atom therein have internucleoside linkages that are formed by short chain alkyl or cycloalkyl internucleoside linkages, mixed heteroatoms and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones: alkene containing backbones: sulfamate backbones: methyleneimino and methylenehydrazino backbones: sulfonate and sulfonamide backbones: amide backbones; and others having mixed N, O, S and CH2 component parts.

In some embodiments, the modified mRNA and/or RNAi molecules described herein include nucleic acids with phosphorothioate internucleoside linkages and oligonucleosides with heteroatom internucleoside linkage, and in particular —CH2-NH—CH2-, —CH2-N(CH3)-O—CH2-[known as a methylene (methylimino) or MMI], —CH2-O—N(CH3)-CH2-, —CH2-N(CH3)-N(CH3)-CH2- and —N(CH3)-CH2-CH2-[wherein the native phosphodiester internucleoside linkage is represented as —O—P—O-CH2-] of the above-referenced U.S. Pat. No. 5,489,677, and the amide backbones of the above-referenced U.S. Pat. No. 5,602,240, both of which are herein incorporated by reference in their entirety. In some embodiments, the nucleic acid sequences featured herein have morpholino backbone structures of the above-referenced U.S. Pat. No. 5,034,506, herein incorporated by reference in its entirety.

Modified mRNA and/or RNAi molecules described herein can also contain one or more substituted sugar moieties. The nucleic acids featured herein can include one of the following at the 2′ position: H (deoxyribose); OH (ribose); F; O-, S-, or N-alkyl; O-, S-, or N-alkenyl; O-, S- or N-alkynyl; or O-alkyl-O-alkyl, wherein the alkyl, alkenyl and alkynyl can be substituted or unsubstituted C1 to C10 alkyl or C2 to C10 alkenyl and alkynyl. Exemplary modifications include O[(CH2)nO]mCH3, O(CH2).nOCH3, O(CH2)nNH2, O(CH2)nCH3, O(CH2)nONH2, and O(CH2)nON[(CH2)nCH3)]2, where n and m are from 1 to about 10. In some embodiments, modified RNAs include one of the following at the 2′ position: C1 to C10 lower alkyl, substituted lower alkyl, alkaryl, aralkyl, O-alkaryl or O-aralkyl, SH, SCH3, OCN, C1, Br, CN, CF3, OCF3, SOCH3, SO2CH3, ONO2, NO2, N3, NH2, heterocycloalkyl, heterocycloalkaryl, aminoalkylamino, polyalkylamino, substituted silyl, a reporter group, an intercalator, a group for improving the pharmacokinetic properties of an RNA, or a group for improving the pharmacodynamic properties of a modified RNA, and other substituents having similar properties. In some embodiments, the modification includes a 2′ methoxyethoxy (2′-O—CH2CH2OCH3, also known as 2′-O-(2-methoxyethyl) or 2′-MOE) (Martin et al., Helv. Chim. Acta, 1995, 78:486-504)i.e., an alkoxy-alkoxy group. Another exemplary modification is 2′-dimethylaminooxyethoxy, i.e., a O(CH2)2ON(CH3)2 group, also known as 2′-DMAOE, and 2′-dimethylaminoethoxyethoxy (also known in the art as 2′-O-dimethylaminoethoxyethyl or 2′-DMAEOE), i.e., 2′-O-CH2-O-CH2-N(CH2)2.

Other exemplary modifications include 2′-methoxy (2′-OCH3), 2′-aminopropoxy (2′-OCH2CH2CH2NH2) and 2′-fluoro (2′-F). Similar modifications can also be made at other positions on the nucleic acid sequence, particularly the 3′ position of the sugar on the 3′ terminal nucleotide or in 2′-5′ linked nucleotides and the 5′ position of 5′ terminal nucleotide. A modified RNA can also have sugar mimetics such as cyclobutyl moieties in place of the pentofuranosyl sugar.

As non-limiting examples, modified mRNA and/or RNAi molecules described herein can include at least one modified nucleoside including a 2′-O-methyl modified nucleoside, a nucleoside comprising a 5′ phosphorothioate group, a 2′-amino-modified nucleoside, 2′-alkyl-modified nucleoside, morpholino nucleoside, a phosphoramidate or a non-natural base comprising nucleoside, or any combination thereof.

In some embodiments, the at least one modified nucleoside is selected from the group consisting of N⁶-methyladenosine (m⁶A), 5-methoxyuridine (5 moU), inosine (I), 5-methylcytosine (m⁵C), pseudouridine (Ψ), 5-hydroxymethylcytosine (hm⁵C), and N¹-methyladenosine (m¹A), N1-methylpseudouridine (me(1)ψ), 5-methylcytidine (5mC), 3,2′-O-dimethyluridine (m4U), 2-thiouridine (s2U), 2′ fluorouridine, 2′-O-methyluridine (Um), 2′ deoxyuridine (2′ dU), 4-thiouridine (s4U), 5-methyluridine (m5U), 2′-O-methyladenosine (m6A), N6,2′-O-dimethyladenosine (m6Am), N6,N6,2′-O-trimethyladenosine (m62Am), 2′-O-methylcytidine (Cm), 7-methylguanosine (m7G), 2′-O-methylguanosine (Gm), N2,7-dimethylguanosine (m2,7G), N2,N2,7-trimethylguanosine (m2,2,7G), and inosine (I). In some embodiments, the at least one modified nucleoside is 5-methoxyuridine (5 moU)).

In some embodiments, a modified mRNA or RNAi molecule comprises at least one nucleoside (“base”) modification or substitution. Modified nucleosides include other synthetic and natural nucleobases such as inosine, xanthine, hypoxanthine, nubularine, isoguanisine, tubercidine, 2-(halo)adenine, 2-(alkyl)adenine, 2-(propyl)adenine, 2 (amino)adenine, 2-(aminoalkyl)adenine, 2 (aminopropyl)adenine, 2 (methylthio) N6 (isopentenyl)adenine, 6 (alkyl)adenine, 6 (methyl)adenine, 7 (deaza)adenine, 8 (alkenyl)adenine, 8-(alkyl)adenine, 8 (alkynyl)adenine, 8 (amino)adenine, 8-(halo)adenine, 8-(hydroxyl)adenine, 8 (thioalkyl)adenine, 8-(thiol)adenine, N6-(isopentyl)adenine, N6 (methyl)adenine, N6, N6 (dimethyl)adenine, 2-(alkyl)guanine, 2 (propyl)guanine, 6-(alkyl)guanine, 6 (methyl)guanine, 7 (alkyl)guanine, 7 (methyl)guanine, 7 (deaza)guanine, 8 (alkyl)guanine, 8-(alkenyl)guanine, 8 (alkynyl)guanine, 8-(amino)guanine, 8 (halo)guanine, 8-(hydroxyl)guanine, 8 (thioalkyl)guanine, 8-(thiol)guanine, N (methyl)guanine, 2-(thio)cytosine, 3 (deaza) 5 (aza)cytosine, 3-(alkyl)cytosine, 3 (methyl)cytosine, 5-(alkyl)cytosine, 5-(alkynyl)cytosine, 5 (halo)cytosine, 5 (methyl)cytosine, 5 (propynyl)cytosine, 5 (propynyl)cytosine, 5 (trifluoromethyl)cytosine, 6-(azo)cytosine, N4 (acetyl)cytosine, 3 (3 amino-3 carboxypropyl)uracil, 2-(thio)uracil, 5 (methyl) 2 (thio)uracil, 5 (methylaminomethyl)-2 (thio)uracil, 4-(thio)uracil, 5 (methyl) 4 (thio)uracil, 5 (methylaminomethyl)-4 (thio)uracil, 5 (methyl) 2,4 (dithio)uracil, 5 (methylaminomethyl)-2,4 (dithio)uracil, 5 (2-aminopropyl)uracil, 5-(alkyl)uracil, 5-(alkynyl)uracil, 5-(allylamino)uracil, 5 (aminoallyl)uracil, 5 (aminoalkyl)uracil, 5 (guanidiniumalkyl)uracil, 5 (1,3-diazole-1-alkyl)uracil, 5-(cyanoalkyl)uracil, 5-(dialkylaminoalkyl)uracil, 5 (dimethylaminoalkyl)uracil, 5-(halo)uracil, 5-(methoxy)uracil, uracil-5 oxyacetic acid, 5 (methoxycarbonylmethyl)-2-(thio)uracil, 5 (methoxycarbonyl-methyl)uracil, 5 (propynyl)uracil, 5 (propynyl)uracil, 5 (trifluoromethyl)uracil, 6 (azo)uracil, dihydrouracil, N3 (methyl)uracil, 5-uracil (i.e., pseudouracil), 2 (thio)pseudouracil, 4 (thio)pseudouracil, 2,4-(dithio)psuedouracil, 5-(alkyl)pseudouracil, 5-(methyl)pseudouracil, 5-(alkyl)-2-(thio)pseudouracil, 5-(methyl)-2-(thio)pseudouracil, 5-(alkyl)-4 (thio)pseudouracil, 5-(methyl)-4 (thio)pseudouracil, 5-(alkyl)-2,4 (dithio)pseudouracil, 5-(methyl)-2,4 (dithio)pseudouracil, 1 substituted pseudouracil, 1 substituted 2(thio)-pseudouracil, 1 substituted 4 (thio)pseudouracil, 1 substituted 2,4-(dithio)pseudouracil, 1 (aminocarbonylethylenyl)-pseudouracil, 1 (aminocarbonylethylenyl)-2(thio)-pseudouracil, 1 (aminocarbonylethylenyl)-4 (thio)pseudouracil, 1 (aminocarbonylethylenyl)-2,4-(dithio)pseudouracil, 1 (aminoalkylaminocarbonylethylenyl)-pseudouracil, 1 (aminoalkylamino-carbonylethylenyl)-2(thio)-pseudouracil, 1 (aminoalkylaminocarbonylethylenyl)-4 (thio)pseudouracil, 1 (aminoalkylaminocarbonylethylenyl)-2,4-(dithio)pseudouracil, 1,3-(diaza)-2-(oxo)-phenoxazin-1-yl, 1-(aza)-2-(thio)-3-(aza)-phenoxazin-1-yl, 1,3-(diaza)-2-(oxo)-phenthiazin-1-yl, 1-(aza)-2-(thio)-3-(aza)-phenthiazin-1-yl, 7-substituted 1,3-(diaza)-2-(oxo)-phenoxazin-1-yl, 7-substituted 1-(aza)-2-(thio)-3-(aza)-phenoxazin-1-yl, 7-substituted 1,3-(diaza)-2-(oxo)-phenthiazin-1-yl, 7-substituted 1-(aza)-2-(thio)-3-(aza)-phenthiazin-1-yl, 7-(aminoalkylhydroxy)-1,3-(diaza)-2-(oxo)-phenoxazin-1-yl, 7-(aminoalkylhydroxy)-1-(aza)-2-(thio)-3-(aza)-phenoxazin-1-yl, 7-(aminoalkylhydroxy)-1,3-(diaza)-2-(oxo)-phenthiazin-1-yl, 7-(aminoalkylhydroxy)-1-(aza)-2-(thio)-3-(aza)-phenthiazin-1-yl, 7-(guanidiniumalkylhydroxy)-1,3-(diaza)-2-(oxo)-phenoxazin-1-yl, 7-(guanidiniumalkylhydroxy)-1-(aza)-2-(thio)-3-(aza)-phenoxazin-1-yl, 7-(guanidiniumalkylhydroxy)-1,3-(diaza)-2-(oxo)-phenthiazin-1-yl, 7-(guanidiniumalkylhydroxy)-1-(aza)-2-(thio)-3-(aza)-phenthiazin-1-yl, 1,3,5-(triaza)-2,6-(dioxa)-naphthalene, inosine, xanthine, hypoxanthine, nubularine, tubercidine, isoguanisine, inosinyl, 2-aza-inosinyl, 7-deaza-inosinvl, nitroimidazolyl, nitropyrazolyl, nitrobenzimidazolyl, nitroindazolyl, aminoindolyl, pyrrolopyrimidinyl, 3-(methyl)isocarbostyrilyl, 5-(methyl)isocarbostyrilyl, 3-(methyl)-7-(propynyl)isocarbostyrilyl, 7-(aza)indolyl, 6-(methyl)-7-(aza)indolyl, imidizopyridinyl, 9-(methyl)-imidizopyridinyl, pyrrolopyrizinyl, isocarbostyrilyl, 7-(propynyl)isocarbostyrilyl, propynyl-7-(aza)indolyl, 2,4,5-(trimethyl)phenyl, 4-(methyl)indolyl, 4,6-(dimethyl)indolyl, phenyl, napthalenyl, anthracenyl, phenanthracenyl, pyrenyl, stilbenzyl, tetracenyl, pentacenyl, difluorotolyl, 4-(fluoro)-6-(methyl)benzimidazole, 4-(methyl)benzimidazole, 6-(azo)thymine, 2-pyridinone, 5 nitroindole, 3 nitropyrrole, 6-(aza)pyrimidine, 2 (amino)purine, 2,6-(diamino)purine, 5 substituted pyrimidines. N2-substituted purines, N6-substituted purines, 06-substituted purines, substituted 1,2,4-triazoles, pyrrolo-pyrimidin-2-on-3-yl, 6-phenyl-pyrrolo-pyrimidin-2-on-3-yl, para-substituted-6-phenyl-pyrrolo-pyrimidin-2-on-3-yl, ortho-substituted-6-phenyl-pyrrolo-pyrimidin-2-on-3-yl, bis-ortho-substituted-6-phenyl-pyrrolo-pyrimidin-2-on-3-yl, para-(aminoalkylhydroxy)-6-phenyl-pyrrolo-pyrimidin-2-on-3-yl, ortho-(aminoalkylhydroxy)-6-phenyl-pyrrolo-pyrimidin-2-on-3-yl, bis-ortho-(aminoalkylhydroxy)-6-phenyl-pyrrolo-pyrimidin-2-on-3-yl, pyridopyrimidin-3-yl, 2-oxo-7-amino-pyridopyrimidin-3-yl, 2-oxo-pyridopyrimidine-3-yl, or any O-alkylated or N-alkylated derivatives thereof. Modified nucleosides also include natural bases that comprise conjugated moieties, e.g. a ligand.

Further modified nucleobases include those disclosed in U.S. Pat. No. 3,687,808, those disclosed in Modified Nucleosides in Biochemistry, Biotechnology and Medicine, Herdewijn, P. ed. Wiley-VCH, 2008; those disclosed in Int. Appl. No. PCT/US09/038,425, filed Mar. 26, 2009; those disclosed in The Concise Encyclopedia Of Polymer Science And Engineering, pages 858-859, Kroschwitz, J. L. ed. John Wiley & Sons, 1990, and those disclosed by Englisch et al., Angewandte Chemie, International Edition, 1991, 30, 613.

Representative U.S. patents that teach the preparation of certain of the above noted modified nucleobases as well as other modified nucleobases include, but are not limited to, the above noted U.S. Pat. No. 3,687,808, as well as U.S. Pat. Nos. 4,845,205; 5,130,30; 5,134,066; 5,175,273; 5,367,066; 5,432,272; 5,457,187; 5,457,191; 5,459,255; 5,484,908; 5,502,177; 5,525,711; 5,552,540; 5,587,469; 5,594,121, 5,596,091; 5,614,617; 5,681,941; 6,015,886; 6,147,200; 6,166,197; 6,222,025; 6,235,887; 6,380,368; 6,528,640; 6,639,062; 6,617,438; 7,045,610; 7,427,672; and 7,495,088, each of which is herein incorporated by reference in its entirety, and U.S. Pat. No. 5,750,692, also herein incorporated by reference in its entirety.

Another modification for use with the modified mRNA and/or RNAi molecules described herein involves chemically linking to the RNA one or more ligands, moieties or conjugates that enhance the activity, cellular distribution or cellular uptake of the RNA. Ligands can be particularly useful where, for example, a modified mRNA or RNAi is administered in vivo. Such moieties include but are not limited to lipid moieties such as a cholesterol moiety (Letsinger et al., Proc. Natl. Acid. Sci. USA, 1989, 86: 6553-6556, herein incorporated by reference in its entirety), cholic acid (Manoharan et al., Biorg. Med. Chem. Let., 1994, 4:1053-1060, herein incorporated by reference in its entirety), a thioether, e.g., beryl-S-tritylthiol (Manoharan et al., Ann. N.Y. Acad. Sci., 1992, 660:306-309: Manoharan et al., Biorg. Med. Chem. Let., 1993, 3:2765-2770, each of which is herein incorporated by reference in its entirety), a thiocholesterol (Oberhauser et al., Nucl. Acids Res., 1992, 20:533-538, herein incorporated by reference in its entirety), an aliphatic chain, e.g., dodecandiol or undecyl residues (Saison-Behmoaras et al., EMBO J, 1991, 10:1111-1118; Kabanov et al., FEBS Lett., 1990, 259:327-330: Svinarchuk et al., Biochimie, 1993, 75:49-54, each of which is herein incorporated by reference in its entirety), a phospholipid, e.g., di-hexadecyl-rac-glycerol or triethyl-ammonium 1,2-di-O-hexadecyl-rac-glycero-3-phosphonate (Manoharan et al., Tetrahedron Lett., 1995, 36:3651-3654; Shea et al., Nucl. Acids Res., 1990, 18:3777-3783, each of which is herein incorporated by reference in its entirety), a polyamine or a polyethylene glycol chain (Manoharan et al., Nucleosides & Nucleotides, 1995, 14:969-973, herein incorporated by reference in its entirety), or adamantane acetic acid (Manoharan et al., Tetrahedron Lett., 1995, 36:3651-3654, herein incorporated by reference in its entirety), a palmityl moiety (Mishra et al., Biochim. Biophys. Acta, 1995, 1264:229-237, herein incorporated by reference in its entirety), or an octadecylamine or hexylamino-carbonyloxycholesterol moiety (Crooke et al., J. Pharmacol. Exp. Ther., 1996, 277:923-937, herein incorporated by reference in its entirety).

The modified mRNA and/or RNAi molecule described herein can further comprise a 5′ cap. In some embodiments of the aspects described herein, the modified mRNA or RNAi molecule comprises a 5′ cap comprising a modified guanine nucleotide that is linked to the 5′ end of an RNA molecule using a 5′-5′ triphosphate linkage. As used herein, the term “5′ cap” is also intended to encompass other 5′ cap analogs including, e.g., 5′ diguanosine cap, tetraphosphate cap analogs having a methylene-bis(phosphonate) moiety (see e.g., Rydzik, A M et al., (2009) Org Biomol Chem 7(22):4763-76), dinucleotide cap analogs having a phosphorothioate modification (see e.g., Kowalska, J. et al., (2008) RNA 14(6):1119-1131), cap analogs having a sulfur substitution for a non-bridging oxygen (see e.g., Grudzien-Nogalska, E. et al., (2007) RNA 13(10): 1745-1755), N7-benzylated dinucleoside tetraphosphate analogs (see e.g., Grudzien, E. et al., (2004) RNA 10(9):1479-1487), or anti-reverse cap analogs (see e.g., Jemielity, J. et al., (2003) RNA 9(9): 1108-1122 and Stepinski, J. et al., (2001) RNA 7(10):1486-1495). In one such embodiment, the 5′ cap analog is a 5′ diguanosine cap. In some embodiments, the modified RNA does not comprise a 5′ triphosphate.

The 5′ cap is important for recognition and attachment of an mRNA to a ribosome to initiate translation. The 5′ cap also protects the modified mRNA or RNAi from 5′ exonuclease mediated degradation. It is not an absolute requirement that a modified mRNA or RNAi molecule comprises a 5′ cap, and thus in other embodiments the modified mRNA or RNAi molecule lacks a 5′ cap. However, due to the longer half-life of the modified mRNA comprising a 5′ cap and the increased efficiency of translation, modified RNAs comprising a 5′ cap are preferred herein.

The modified mRNA molecules described herein can further comprise a 5′ and/or 3′ untranslated region (UTR). Untranslated regions are regions of the RNA before the start codon (5′) and after the stop codon (3′), and are therefore not translated by the translation machinery. Modification of an RNA molecule with one or more untranslated regions can improve the stability of an mRNA, since the untranslated regions can interfere with ribonucleases and other proteins involved in RNA degradation. In addition, modification of an RNA with a 5′ and/or 3′ untranslated region can enhance translational efficiency by binding proteins that alter ribosome binding to an mRNA. Modification of an RNA with a 3′ UTR can be used to maintain a cytoplasmic localization of the RNA, permitting translation to occur in the cytoplasm of the cell. In one embodiment, the modified mRNA described herein does not comprise a 5′ or 3′ UTR. In another embodiment, the modified mRNAs comprise either a 5′ or 3′ UTR. In another embodiment, the modified mRNA described herein comprises both a 5′ and a 3′ UTR. In one embodiment, the 5′ and/or 3′ UTR is selected from an mRNA known to have high stability in the cell (e.g., a murine alpha-globin 3′ UTR). In some embodiments, the 5′ UTR, the 3′ UTR, or both comprise one or more modified nucleosides.

In some embodiments, the modified mRNA described herein further comprises a Kozak sequence. The “Kozak sequence” refers to a sequence on eukaryotic mRNA having the consensus (gcc)gccRccAUGG (SEQ ID NO: 92), where R is a purine (adenine or guanine) three bases upstream of the start codon (AUG), which is followed by another ‘G’. The Kozak consensus sequence is recognized by the ribosome to initiate translation of a polypeptide. Typically, initiation occurs at the first AUG codon encountered by the translation machinery that is proximal to the 5′ end of the transcript. However, in some cases, this AUG codon can be bypassed in a process called leaky scanning. The presence of a Kozak sequence near the AUG codon will strengthen that codon as the initiating site of translation, such that translation of the correct polypeptide occurs. Furthermore, addition of a Kozak sequence to a modified RNA will promote more efficient translation, even if there is no ambiguity regarding the start codon. Thus, in some embodiments, the modified RNAs described herein further comprise a Kozak consensus sequence at the desired site for initiation of translation to produce the correct length polypeptide. In some such embodiments, the Kozak sequence comprises one or more modified nucleosides.

In some embodiments, the modified mRNA and/or RNAi molecules described herein further comprise a “poly (A) tail”, which refers to a 3′ homopolymeric tail of adenine nucleotides, which can vary in length (e.g., at least 5 adenine nucleotides) and can be up to several hundred adenine nucleotides). The inclusion of a 3′ poly(A) tail can protect the modified RNA from degradation in the cell, and also facilitates extra-nuclear localization to enhance translation efficiency. In some embodiments, the poly(A) tail comprises between 1 and 500 adenine nucleotides; in other embodiments the poly(A) tail comprises at least 5, at least 10, at least 20, at least 30, at least 40, at least 50, at least 60, at least 70, at least 80, at least 90, at least 100, at least 110, at least 120, at least 130, at least 140, at least 150, at least 160, at least 170, at least 180, at least 190, at least 200, at least 225, at least 250, at least 275, at least 300, at least 325, at least 350, at least 375, at least 400, at least 425, at least 450, at least 475, at least 500 adenine nucleotides or more. In one embodiment, the poly(A) tail comprises between 1 and 150 adenine nucleotides. In another embodiment, the poly(A) tail comprises between 90 and 120 adenine nucleotides. In some such embodiments, the poly(A) tail comprises one or more modified nucleosides.

It is contemplated that one or more modifications to the modified mRNA and/or RNAi molecules described herein permit greater stability of the modified RNA molecule in a cell. To the extent that such modifications permit translation and/or either reduce or do not exacerbate a cell's innate immune or interferon response to the modified RNA with the modification, such modifications are specifically contemplated for use herein. Generally, the greater the stability of a modified mRNA, the more protein can be produced from that modified mRNA. Typically, the presence of AU-rich regions in mammalian mRNAs tend to destabilize transcripts, as cellular proteins are recruited to AU-rich regions to stimulate removal of the poly(A) tail of the transcript. Loss of a poly(A) tail of a modified RNA can result in increased RNA degradation. Thus, in one embodiment, a modified RNA as described herein does not comprise an AU-rich region. In some embodiments, the 3′ UTR substantially lacks AUUUA sequence elements.

Complexes and Nanoparticles comprising Cell-Penetrating Peptides

In some aspects, the invention provides complexes and nanoparticles comprising cell-penetrating peptides for delivering one or more mRNA into a cell. In some embodiments, cell-penetrating peptides are complexed with the one or more mRNA. In some embodiments, the cell-penetrating peptides are non-covalently complexed with at least one of the one or more mRNA. In some embodiments, the cell-penetrating peptides are non-covalently complexed with each of the one or more mRNA. In some embodiments, the cell-penetrating peptides are covalently complexed with at least one of the one or more mRNA. In some embodiments, the cell-penetrating peptides are covalently complexed with each of the one or more mRNA. In some embodiments, the mRNA encodes a protein, such as a therapeutic protein. In some embodiments, the mRNA is modified (e.g., wherein at least one modified nucleoside is 5-methoxyuridine (5 moU)). In some embodiments, the complex and/or nanoparticle further comprises an RNAi, or is administered in combination with an RNAi (e.g., administered in combination with a complex or nanoparticle comprising cell-penetrating peptides for delivering the RNAi into a cell). In some embodiments, the RNAi targets an endogenous gene, e.g., a disease-associated endogenous gene. In some embodiments, the RNAi targets an exogenous gene. In some embodiments, the complex and/or nanoparticle comprises a first mRNA encoding a first protein, and a second mRNA encoding a second protein. In some embodiments, the complex and/or nanoparticle comprises a first RNAi (e.g, siRNA) targeting a first endogenous gene, and a second RNAi (e.g., siRNA) targeting a second endogenous gene. In some embodiments, the complex and/or nanoparticle comprises an mRNA encoding a protein, such as a therapeutic protein and an RNAi (e.g., siRNA) targeting an endogenous gene. In some embodiments, the RNAi is a therapeutic RNAi targeting an endogenous gene involved in a disease or condition. In some embodiments, the therapeutic RNAi targets a disease-associated form of the endogenous gene (e.g., a gene encoding a mutant protein, or a gene resulting in abnormal expression of a protein).

In some aspects, the invention provides complexes and nanoparticles comprising cell-penetrating peptides for delivering one or more RNAi (e.g., siRNA) into a cell. In some embodiments, cell-penetrating peptides are complexed with the one or more RNAi (e.g., siRNA). In some embodiments, the cell-penetrating peptides are non-covalently complexed with at least one of the one or more RNAi (e.g., siRNA). In some embodiments, the cell-penetrating peptides are non-covalently complexed with each of the one or more RNAi (e.g., siRNA). In some embodiments, the cell-penetrating peptides are covalently complexed with at least one of the one or more RNAi (e.g., siRNA). In some embodiments, the cell-penetrating peptides are covalently complexed with each of the one or more RNAi (e.g., siRNA). In some embodiments, the RNAi (e.g., siRNA) targets an endogenous gene. In some embodiments, the endogenous gene is involved in a disease or a condition. In some embodiments, the RNAi targets a disease-associated form of the endogenous gene (e.g., a gene encoding a mutant protein, or a gene resulting in abnormal expression of a protein). In some embodiments, the RNAi targets an exogenous gene. In some embodiments, the complex and/or nanoparticle comprises a first RNAi (e.g., siRNA) targeting a first endogenous gene, and a second RNAi (e.g., siRNA) targeting a second endogenous gene.

### Cell-Penetrating Peptides

The cell-penetrating peptides in the mRNA delivery complexes or nanoparticles of the present invention are capable of forming stable complexes and nanoparticles with various mRNA. Any of the cell-penetrating peptides in any of the mRNA delivery complexes or nanoparticles described herein may comprise or consist of any of the cell-penetrating peptide sequences described in this section.

In some embodiments, an mRNA delivery complex or nanoparticle described herein comprises a cell-penetrating peptide selected from the group consisting of CADY, PEP-1, PEP-2, MPG, VEPEP-3 peptides (used herein interchangeably with ADGN-103 peptides), VEPEP-4 peptides (used herein interchangeably with ADGN-104 peptides), VEPEP-5 peptides (used herein interchangeably with ADGN-105 peptides), VEPEP-6 peptides (used herein interchangeably with ADGN-106 peptides), VEPEP-9 peptides (used herein interchangeably with ADGN-109 peptides), and ADGN-100 peptides. In some embodiments, the cell-penetrating peptide is present in an mRNA delivery complex. In some embodiments, the cell-penetrating peptide is present in an mRNA delivery complex present in the core of a nanoparticle. In some embodiments, the cell-penetrating peptide is present in the core of a nanoparticle. In some embodiments, the cell-penetrating peptide is present in the core of a nanoparticle and is associated with an mRNA. In some embodiments, the cell-penetrating peptide is present in an intermediate layer of a nanoparticle. In some embodiments, the cell-penetrating peptide is present in the surface layer of a nanoparticle. In some embodiments, the cell-penetrating peptide is linked to a targeting moiety. In some embodiments, the linkage is covalent. In some embodiments, the covalent linkage is by chemical coupling. In some embodiments, the covalent linkage is by genetic methods. WO2014/053879 discloses VEPEP-3 peptides; WO2014/053881 discloses VEPEP-4 peptides; WO2014/053882 discloses VEPEP-5 peptides; WO2012/137150 discloses VEPEP-6 peptides; WO2014/053880 discloses VEPEP-9 peptides; WO 2016/102687 discloses ADGN-100 peptides; US2010/0099626 discloses CADY peptides; and. U.S. Pat. No. 7,514,530 discloses MPG peptides; the disclosures of which are hereby incorporated herein by reference in their entirety.

In some embodiments, an mRNA delivery complex or nanoparticle described herein comprises a VEPEP-3 cell-penetrating peptide comprising the amino acid sequence X₁X₂X₃X₄X₅X₂X₃X₄X₆X₇X₃X₈X₉X₁₀X₁₁X₁₂X₁₃ (SEQ ID NO: 1), wherein X₁ is beta-A or S, X₂ is K, R or L (independently from each other), X₃ is F or W (independently from each other). X₄ is F, W or Y (independently from each other), X₅ is E, R or S, X₆ is R, T or S, X₇ is E, R, or S, X₈ is none, F or W, X₉ is P or R, X₁₀ is R or L, X₁ is K, W or R, X₁₂ is R or F, and X₁₃ is R or K. In some embodiments, the VEPEP-3 peptide comprises the amino acid sequence X₁X₂WX₄EX₂WX₄X₆X₇X₃PRX₁₁RX₁₃ (SEQ ID NO: 2), wherein X₁ is beta-A or S, X₂ is K, R or L, X₃ is F or W, X₄ is F, W or Y, X₅ is E, R or S, X₆ is R, T or S, X₇ is E, R, or S, X₈ is none, F or W, X₉ is P or R, X₁₀ is R or L, X₁ is K, W or R, X₁₂ is R or F, and X₁₃ is R or K. In some embodiments, the VEPEP-3 peptide comprises the amino acid sequence X₁KWFERWFREWPRKRR (SEQ ID NO: 3), X₁KWWERWWREWPRKRR (SEQ ID NO: 4), X₁KWWERWWREWPRKRK (SEQ ID NO: 5), X₁RWWEKWWTRWPRKRK (SEQ ID NO: 6), or X₁RWYEKWYTEFPRRRR (SEQ ID NO: 7), wherein X₁ is beta-A or S. In some embodiments, the VEPEP-3 peptide comprises the amino acid sequence of any one of SEQ ID NOs: 1-7, wherein the cell-penetrating peptide is modified by replacement of the amino acid in position 10 by a non-natural amino acid, addition of a non-natural amino acid between the amino acids in positions 2 and 3, and addition of a hydrocarbon linkage between the two non-natural amino acids. In some embodiments, the VEPEP-3 peptide comprises the amino acid sequence X₁KX₁₄WWERWWRX₁₄WPRKRK (SEQ ID NO: 8), wherein X₁ is beta-A or S and X₁₄ is a non-natural amino acid, and wherein there is a hydrocarbon linkage between the two non-natural amino acids. In some embodiments, the VEPEP-3 peptide comprises the amino acid sequence X₁X₂X₃WX₅X₁₀X₃WX₆X₇WX₈X₉X₁₀WX₂R (SEQ ID NO: 9), wherein X₁ is beta-A or S, X₂ is K, R or L, X₃ is F or W, X₅ is R or S, X₆ is R or S, X₇ is R or S, X₈ is F or W, X₉ is R or P, X₁₀ is L or R, and X₁₂ is R or F. In some embodiments, the VEPEP-3 peptide comprises the amino acid sequence X₁RWWRLWWRSWFRLWRR (SEQ ID NO: 10), X₁LWWRRWWSRWWPRWRR (SEQ ID NO: 11), X₁LWWSRWWRSWFRLWFR (SEQ ID NO: 12), or X₁KFWSRFWRSWFRLWRR (SEQ ID NO: 13), wherein X₁ is beta-A or S. In some embodiments, the VEPEP-3 peptide comprises the amino acid sequence of any one of SEQ ID NOs: 1 and 9-13, wherein the cell-penetrating peptide is modified by replacement of the amino acids in position 5 and 12 by non-natural amino acids, and addition of a hydrocarbon linkage between the two non-natural amino acids. In some embodiments, the VEPEP-3 peptide comprises the amino acid sequence X₁RWWX₁₄LWWRSWX₁₄RLWRR (SEQ ID NO: 14), wherein X₁ is a beta-alanine or a serine and X₁₄ is a non-natural amino acid, and wherein there is a hydrocarbon linkage between the two non-natural amino acids. In some embodiments, the VEPEP-3 peptide is present in an mRNA delivery complex. In some embodiments, the VEPEP-3 peptide is present in an mRNA delivery complex in the core of a nanoparticle. In some embodiments, the VEPEP-3 peptide is present in the core of a nanoparticle. In some embodiments, the VEPEP-3 peptide is present in the core of a nanoparticle and is associated with an mRNA. In some embodiments, the VEPEP-3 peptide is present in an intermediate layer of a nanoparticle. In some embodiments, the VEPEP-3 peptide is present in the surface layer of a nanoparticle. In some embodiments, the VEPEP-3 peptide is linked to a targeting moiety. In some embodiments, the linkage is covalent. In some embodiments, the covalent linkage is by chemical coupling. In some embodiments, the covalent linkage is by genetic methods.

In some embodiments, an mRNA delivery complex or nanoparticle described herein comprises a VEPEP-6 cell-penetrating peptide. In some embodiments, the VEPEP-6 peptide comprises an amino acid sequence selected from the group consisting of X₁LX₂RALWX₉LX₃X₉X₄LWX₉LX₅X₆X₇X₈ (SEQ ID NO: 15), X₁LX₂LARWX₉LX₃X₉X₄LWX₉LX₅X₆X-X₈ (SEQ ID NO: 16) and X₁LX₂ARLWX₉LX₃X₉X₄LWX₉LX₅X₆X₇X₈ (SEQ ID NO: 17), wherein X₁ is beta-A or S, X₂ is F or W, X₃ is L, W, C or I, X₄ is S, A, N or T, X₅ is L or W, X₆ is W or R, X₇ is K or R, X₈ is A or none, and X₉ is R or S. In some embodiments, the VEPEP-6 peptide comprises the amino acid sequence X₁LX₂RALWRLX₃RX₄LWRLX₅X₆X₇X₈ (SEQ ID NO: 18), wherein X₁ is beta-A or S, X₂ is F or W, X₃ is L, W, C or I, X₄ is S, A, N or T, X₅ is L or W, X₆ is W or R, X₇ is K or R, and X₈ is A or none. In some embodiments, the VEPEP-6 peptide comprises the amino acid sequence X₁LX₂RALWRLX₃RX₄LWRLX₅X₆KX₇ (SEQ ID NO: 19), wherein X₁ is beta-A or S, X₂ is F or W, X₃ is L or W, X₄ is S. A or N, X₅ is L or W, X₆ is W or R, X₇ is A or none. In some embodiments, the VEPEP-6 peptide comprises an amino acid sequence selected from the group consisting of X₁FRALWRLLRX₂LWRLLWX₃ (SEQ ID NO: 20), X₁LWRALWRLWRX₂LWRLLWX₃A (SEQ ID NO: 21), X₁LWRALWRLX₄RX₂LWRLWRX₃A (SEQ ID NO: 22), X₁LWRALWRLWRX₂LWRLWRX₃A (SEQ ID NO: 23), X₁LWRALWRLX₅RALWRLLWX₃A (SEQ ID NO: 24), and X₁LWRALWRLX₄RNLWRLLWX₃A (SEQ ID NO: 25), wherein X₁ is beta-A or S, X₂ is S or T, X₃ is K or R, X₄ is L, C or I and X₅ is L or I. In some embodiments, the VEPEP-6 peptide comprises an amino acid sequence selected from the group consisting of Ac-X₁LFRALWRLLRSLWRLLWK-cysteamide (SEQ ID NO: 26), Ac-X₁LWRALWRLWRSLWRLLWKA-cysteamide (SEQ ID NO: 27), Ac-X₁LWRALWRLLRSLWRLWRKA-cysteamide (SEQ ID NO: 28), Ac-X₁LWRALWRLWRSLWRLWRKA-cysteamide (SEQ ID NO: 29), Ac-X₁LWRALWRLLRALWRLLWKA-cysteamide (SEQ ID NO: 30), and Ac-X₁LWRALWRLLRNLWRLLWKA-cysteamide (SEQ ID NO: 31), wherein X₁ is beta-A or S. In some embodiments, the VEPEP-6 peptide comprises the amino acid sequence of any one of SEQ ID NOs: 15-31, further comprising a hydrocarbon linkage between two residues at positions 8 and 12. In some embodiments, the VEPEP-6 peptide comprises an amino acid sequence selected from the group consisting of Ac-X₁LFRALWR_{S}LLRS_{S}LWRLLWK-cysteamide (SEQ ID NO: 32), Ac-X₁LFLARWR_{S}LLRS_{S}LWRLLWK-cysteamide (SEQ ID NO: 33), Ac-X₁LFRALWS_{S}LLRS_{S}LWRLLWK-cysteamide (SEQ ID NO: 34), Ac-X₁LFLARWS_{S}LLRS_{S}LWRLLWK-cysteamide (SEQ ID NO: 35), Ac-X₁LFRALWRLLR_{S}SLWS_{S}LLWK-cysteamide (SEQ ID NO: 36), Ac-X₁LFLARWRLLR_{S}SLWS_{S}LLWK-cysteamide (SEQ ID NO: 37), Ac-X₁LFRALWRLLS_{S}SLWS_{S}LLWK-cysteamide (SEQ ID NO: 38), Ac-X₁LFLARWRLLS_{S}SLWS_{S}LLWK-cysteamide (SEQ ID NO: 39), and Ac-X₁LFAR_{S}LWRLLRS_{S}LWRLLWK-cysteamide (SEQ ID NO: 40), wherein X₁ is beta-A or S and wherein the residues followed by an inferior “S” are those which are linked by said hydrocarbon linkage. In some embodiments, the VEPEP-6 peptide is present in an mRNA delivery complex. In some embodiments, the VEPEP-6 peptide is present in an mRNA delivery complex in the core of a nanoparticle. In some embodiments, the VEPEP-6 peptide is present in the core of a nanoparticle. In some embodiments, the VEPEP-6 peptide is present in the core of a nanoparticle and is associated with an mRNA. In some embodiments, the VEPEP-6 peptide is present in an intermediate layer of a nanoparticle. In some embodiments, the VEPEP-6 peptide is present in the surface layer of a nanoparticle. In some embodiments, the VEPEP-6 peptide is linked to a targeting moiety. In some embodiments, the linkage is covalent. In some embodiments, the covalent linkage is by chemical coupling. In some embodiments, the covalent linkage is by genetic methods.

In some embodiments, an mRNA delivery complex or nanoparticle described herein comprises a VEPEP-9 cell-penetrating peptide comprising the amino acid sequence X₁X₂X₃WWX₄X₅WAX₆X₃X₇X₈X₉X₁₀X₁₁X₁₂WX₁₃R (SEQ ID NO: 41), wherein X₁ is beta-A or S, X₂ is L or none, X₃ is R or none, X₄ is L, R or G, X₅ is R, W or S, X₆ is S, P or T, X₇ is W or P, X₈ is F, A or R, X₉ is S, L, P or R, X₁₀ is R or S, X₁₁ is W or none, X₁₂ is A, R or none and X₁₃ is W or F, and wherein if X₃ is none, then X₂, X₁₁ and X₁₂ are none as well. In some embodiments, the VEPEP-9 peptide comprises the amino acid sequence X₁X₂RWWLRWAX₆RWX₈X₉X₁₀WX₁₂WX₁₃R (SEQ ID NO: 42), wherein X₁ is beta-A or S. X₂ is L or none, X₆ is S or P, X₈ is F or A, X₉ is S, L or P, X₁₀ is R or S, X₁₂ is A or R, and X₁₃ is W or F. In some embodiments, the VEPEP-9 peptide comprises an amino acid sequence selected from the group consisting of X₁LRWWLRWASRWFSRWAWWR (SEQ ID NO: 43), X₁LRWWLRWASRWASRWAWFR (SEQ ID NO: 44), X₁RWWLRWASRWALSWRWWR (SEQ ID NO: 45), X₁RWWLRWASRWFLSWRWWR (SEQ ID NO: 46), X₁RWWLRWAPRWFPSWRWWR (SEQ ID NO: 47), and X₁RWWLRWASRWAPSWRWWR (SEQ ID NO: 48), wherein X₁ is beta-A or S. In some embodiments, the VEPEP-9 peptide comprises the amino acid sequence of X₁WWX₄X₅WAX₆X₇X₈RX₁₀WWR (SEQ ID NO: 49), wherein X₁ is beta-A or S, X₄ is R or G, X₅ is W or S, X₆ is S, T or P, X₇ is W or P, X₈ is A or R, and X₁₀ is S or R. In some embodiments, the VEPEP-9 peptide comprises an amino acid sequence selected from the group consisting of X₁WWRWWASWARSWWR (SEQ ID NO: 50), X₁WWGSWATPRRRWWR (SEQ ID NO: 51), and X₁WWRWWAPWARSWWR (SEQ ID NO: 52), wherein X₁ is beta-A or S. In some embodiments, the VEPEP-9 peptide is present in an mRNA delivery complex. In some embodiments, the VEPEP-9 peptide is present in an mRNA delivery complex in the core of a nanoparticle. In some embodiments, the VEPEP-9 peptide is present in the core of a nanoparticle. In some embodiments, the VEPEP-9 peptide is present in the core of a nanoparticle and is associated with an mRNA. In some embodiments, the VEPEP-9 peptide is present in an intermediate layer of a nanoparticle. In some embodiments, the VEPEP-9 peptide is present in the surface layer of a nanoparticle. In some embodiments, the VEPEP-9 peptide is linked to a targeting moiety. In some embodiments, the linkage is covalent. In some embodiments, the covalent linkage is by chemical coupling. In some embodiments, the covalent linkage is by genetic methods.

In some embodiments, an mRNA delivery complex or nanoparticle described herein comprises an ADGN-100 cell-penetrating peptide comprising the amino acid sequence X₁KWRSX₂X₃X₄RWRLWRX₅X₆X₇X₈SR (SEQ ID NO: 53), wherein X₁ is any amino acid or none, and X₂-X₈ are any amino acid. In some embodiments, the ADGN-100 peptide comprises the amino acid sequence X₁KWRSX₂X₃X₄RWRLWRX₅X₆X₇X₈R (SEQ ID NO: 54), wherein X₁ is βA, S, or none, X₂ is A or V, X₃ is or L, X₄ is W or Y, X₅ is V or S, X₆ is R, V, or A, X₇ is S or L, and X₈ is W or Y. In some embodiments, the ADGN-100 peptide comprises the amino acid sequence KWRSAGWRWRLWRVRSWSR (SEQ ID NO: 55), KWRSALYRWRLWRVRSWSR (SEQ ID NO: 56), KWRSALYRWRLWRSRSWSR (SEQ ID NO: 57), or KWRSALYRWRLWRSALYSR (SEQ ID NO: 58). In some embodiments, the ADGN-100 peptide comprises two residues separated by three or six residues that are linked by a hydrocarbon linkage. In some embodiments, the ADGN-100 peptide comprises the amino acid sequence KWRS_{S}AGWR_{S}WRLWRVRSWSR (SEQ ID NO: 59), KWR_{S}SAGWRWR_{S}LWRVRSWSR (SEQ ID NO: 60), KWRSAGWR_{S}WRLWRVR_{S}SWSR (SEQ ID NO: 61), KWRS_{S}ALYR_{S}WRLWRSRSWSR (SEQ ID NO: 62), KWR_{S}SALYRWR_{S}LWRSRSWSR (SEQ ID NO: 63), KWRSALYR_{S}WRLWRSR_{S}SWSR (SEQ ID NO: 64). KWR_{S}ALYRWR_{S}LWRSSRSWSR (SEQ ID NO: 65), KWRSALYRWRLWRS_{S}RSWS_{S}R (SEQ ID NO: 66), KWR_{S}SALYRWR_{S}LWRSALYSR (SEQ ID NO: 67), KWRS_{S}ALYR_{S}WRLWRSALYSR (SEQ ID NO: 68), KWRSALYRWR_{S}LWRS_{S}ALYSR (SEQ ID NO: 69), or KWRSALYRWRLWRS_{S}ALYS_{S}R (SEQ ID NO: 70), wherein the residues marked with a subscript “S” are linked by a hydrocarbon linkage. In some embodiments, the ADGN-100 peptide is present in an mRNA delivery complex. In some embodiments, the ADGN-100 peptide is present in an mRNA delivery complex in the core of a nanoparticle. In some embodiments, the ADGN-100 peptide is present in the core of a nanoparticle. In some embodiments, the ADGN-100 peptide is present in the core of a nanoparticle and is associated with an mRNA. In some embodiments, the ADGN-100 peptide is present in an intermediate layer of a nanoparticle. In some embodiments, the ADGN-100 peptide is present in the surface layer of a nanoparticle. In some embodiments, the ADGN-100 peptide is linked to a targeting moiety. In some embodiments, the linkage is covalent. In some embodiments, the covalent linkage is by chemical coupling. In some embodiments, the covalent linkage is by genetic methods.

In some embodiments, the CPP described herein (e.g., PEP-1, PEP-2, VEPEP-3 peptide, VEPEP-6 peptide, VEPEP-9 peptide, or ADGN-100 peptide) further comprises one or more moieties linked to the N-terminus of the CPP. In some embodiments, the one or more moieties is covalently linked to the N-terminus of the CPP. In some embodiments, the one or more moieties are selected from the group consisting of an acetyl group, a stearyl group, a fatty acid, a cholesterol, a poly-ethylene glycol, a nuclear localization signal, a nuclear export signal, an antibody or antibody fragment thereof, a peptide, a polysaccharide, and a targeting molecule. In some embodiments, the one or more moieties is an acetyl group and/or a stearyl group. In some embodiments, the CPP comprises an acetyl group and/or a stearyl group linked to its N-terminus. In some embodiments, the CPP comprises an acetyl group linked to its N-terminus. In some embodiments, the CPP comprises a stearyl group linked to its N-terminus. In some embodiments, the CPP comprises an acetyl group and/or a stearyl group covalently linked to its N-terminus. In some embodiments, the CPP comprises an acetyl group covalently linked to its N-terminus. In some embodiments, the CPP comprises a stearyl group covalently linked to its N-terminus.

In some embodiments, the CPP described herein (e.g., PEP-1, PEP-2, VEPEP-3 peptide, VEPEP-6 peptide, VEPEP-9 peptide, or ADGN-100 peptide) further comprises one or more moieties linked to the C-terminus of the CPP. In some embodiments, the one or more moieties is covalently linked to the C-terminus of the CPP. In some embodiments, the one or more moieties are selected from the group consisting of a cysteamide group, a cysteine, a thiol, an amide, a nitrilotriacetic acid, a carboxyl group, a linear or ramified C₁-C₆ alkyl group, a primary or secondary amine, an osidic derivative, a lipid, a phospholipid, a fatty acid, a cholesterol, a poly-ethylene glycol, a nuclear localization signal, a nuclear export signal, an antibody or antibody fragment thereof, a peptide, a polysaccharide, and a targeting molecule. In some embodiments, the one or more moieties is a cysteamide group. In some embodiments, the CPP comprises a cysteamide group linked to its C-terminus. In some embodiments, the CPP comprises a cysteamide group covalently linked to its C-terminus.

In some embodiments, the CPP described herein (e.g., PEP-1, PEP-2, VEPEP-3 peptide, VEPEP-6 peptide, VEPEP-9 peptide, or ADGN-100 peptide) is stapled. “Stapled” as used herein refers to a chemical linkage between two residues in a peptide. In some embodiments, the CPP is stapled, comprising a chemical linkage between two amino acids of the peptide. In some embodiments, the two amino acids linked by the chemical linkage are separated by 3 or 6 amino acids. In some embodiments, two amino acids linked by the chemical linkage are separated by 3 amino acids. In some embodiments, the two amino acids linked by the chemical linkage are separated by 6 amino acids. In some embodiments, each of the two amino acids linked by the chemical linkage is R or S. In some embodiments, each of the two amino acids linked by the chemical linkage is R. In some embodiments, each of the two amino acids linked by the chemical linkage is S. In some embodiments, one of the two amino acids linked by the chemical linkage is R and the other is S. In some embodiments, the chemical linkage is a hydrocarbon linkage.

### Complexes Comprising Cell-Penetrating Peptides

In some embodiments, there is provided an mRNA delivery complex for intracellular delivery of an mRNA comprising a cell-penetrating peptide (e.g., a PEP-1, PEP-2, VEPEP-3, VEPEP-6, VEPEP-9, or ADGN-100 peptide) associated with one or more mRNA. In some embodiments, the association is non-covalent. In some embodiments, the association is covalent.

In some embodiments, at least some of the cell-penetrating peptides in the mRNA delivery complex are linked to a targeting moiety. In some embodiments, the linkage is covalent. In some embodiments, the covalent linkage is by chemical coupling. In some embodiments, the covalent linkage is by genetic methods. In some embodiments, the molar ratio of cell-penetrating peptide to at least one of the one or more mRNA is between about 1:1 and about 100:1, or between about 1:1 and about 50:1, or about 20:1. In some embodiments, the CPP includes, but is not limited to, a PTD-based peptide, an amphipathic peptide, a poly-arginine-based peptide, an MPG peptide, a CADY peptide, a VEPEP peptide (such as a VEPEP-3, VEPEP-6, or VEPEP-9 peptide), an ADGN-100 peptide, a Pep-1 peptide, and a Pep-2 peptide.

In some embodiments, the mRNA delivery complex comprises an mRNA encoding a therapeutic protein. In some embodiments, the tumor suppressor protein corresponds to a tumor-suppressor gene. In some embodiments, the corresponding tumor-suppressor gene includes, without limitation, PTEN, Retinoblastoma RB (or RB1), TP53, TP63, TP73, CDKN2A (INK4A), CDKN1B, CDKN1C, DLD/NP1, HEPACAM, SDHB, SDHD, SFRP1, TCF21, TIG1, MLH1. MSH2, MSH6, WT1, WT2, NF1, NF2N, VHL, KLF4, pVHL, APC, CD95, ST5, YPEL3, ST7, APC, MADR2, BRCA1, BRCA2, Patched, TSC1, TSC2, PALB2, ST14, or VHL. In some embodiments, the tumor suppressor gene is selected from PB1, TSC1, TSC2, BRCA1, BRCA2, PTEN and TP53.

In some embodiments, the mRNA delivery complex comprises an mRNA encoding a therapeutic protein PTEN. In some embodiments, the tumor suppressor protein PTEN is encoded by a human PTEN sequence. In some embodiments, the mRNA comprises a sequence selected from the group consisting of sequences with accession number of BC005821, JF268690. U92436, CR450306, AK024986, AK313581, U96180, and U93051 and NM_000314 in NCBI GenBank.

In some embodiments, the mRNA delivery complex comprises an mRNA encoding a therapeutic protein p53. In some embodiments, the tumor suppressor protein p53 is encoded by a human TP53 sequence. In some embodiments, the mRNA comprises a sequence selected from the group consisting of sequences with accession number of AF052180, NM_000546, AY429684, BT019622, AK223026, DQ186652, DQ186651, DQ186650, DQ186649, DQ186648, DQ263704, DQ286964, DQ191317, DQ401704, AF307851, AM076972, AM076971, AM076970, DQ485152, BC003596, DQ648887, DQ648886, DQ648885, DQ648884, AK225838, M14694, M14695, EF101869, EF101868, EF101867, X01405, AK312568, NM_001126117, NM_001126116, NM_001126115, NM_001126114, NM_001126113, NM_001126112, FJ207420, X60020, X60019, X60018, X60017, X60016, X60015, X60014, X60013, X60011, X60012, X60010, X02469, S66666, AB082923, NM_001126118, JN900492, NM_001276699, NM_001276698, NM_001276697, NM_001276761, NM_001276760, NM_001276696, and NM_001276695 in NCBI GenBank.

In some embodiments, the mRNA delivery complex comprises an mRNA encoding a therapeutic protein BRCA1. In some embodiments, the tumor suppressor protein BRCA1 is encoded by a human BRCA1 sequence. In some embodiments, the mRNA comprises a sequence selected from the group consisting of a sequence with with accession number of NM_007294, NM_007297, NM_007298, NM_007304, NM 007299, NM_007300, BC046142, BC062429, BC072418, AY354539, AY751490, BC085615, BC106746, BC106745, BC114511, BC115037, U14680, AK293762, U68041, BC030969, BC012577, AK316200, DQ363751, DQ333387, DQ333386, Y08864, JN686490, AB621825, BC038947, U64805, and AF005068 in NCBI GenBank.

In some embodiments, the mRNA delivery complex comprises an mRNA encoding a therapeutic protein BRCA2. In some embodiments, the tumor suppressor protein BRCA2 is encoded by a human BRCA2 sequence. In some embodiments, the mRNA comprises a sequence selected from the group consisting of a sequence with with accession number of BC047568, NM_000059, DQ897648, BC026160 in NCBI GenBank.

In some embodiments, the mRNA delivery complex comprises an mRNA encoding a therapeutic protein TSC1. In some embodiments, the tumor suppressor protein TSC1 is encoded by a human TSC1 sequence. In some embodiments, the mRNA comprises a sequence selected from the group consisting of a sequence with with accession number of BC047772, NM_000368, BC070032, AB190910, BC108668, BC121000, NM_001162427, NM_001162426, D87683, and AF013168 in NCBI GenBank.

In some embodiments, the mRNA delivery complex comprises an mRNA encoding a therapeutic protein TSC2. In some embodiments, the tumor suppressor protein TSC2 is encoded by a human TSC2 sequence. In some embodiments, the mRNA comprises a sequence selected from the group consisting of a sequence with with accession number of BC046929, BX647816, AK125096, NM_000548, AB210000, NM_001077183, BC150300, BC025364, NM_001114382, AK094152, AK299343, AK295728, AK295672, AK294548, and X75621 in NCBI GenBank.

In some embodiments, the mRNA delivery complex comprises an mRNA encoding a therapeutic protein Retinoblastoma 1 (RB1). In some embodiments, the tumor suppressor protein RB1 is encoded by a human RB1 sequence. In some embodiments, the mRNA comprises a sequence selected from the group consisting of a sequence with accession number of NM_000321. AY429568. AB208788, M19701, AK291258, L41870, AK307730, AK307125, AK300284, AK299179, M33647, M15400, M28419, BC039060, BC040540, and AF043224 in NCBI GenBank.

In some embodiments, the mRNA delivery complex comprises an mRNA encoding a therapeutic protein, wherein the deficiency of the protein results in a disease or disorder. In some embodiments, the protein is Frataxin. In some embodiments, the protein is alpha 1 antitrypsin. In some embodiments, the protein is factor VIII. In some embodiments, the protein is factor IX.

In some embodiments, there is provided an RNAi (e.g., siRNA) delivery complex for intracellular delivery of an RNAi (e.g., siRNA) comprising a cell-penetrating peptide (e.g., a PEP-1, PEP-2, VEPEP-3, VEPEP-6, VEPEP-9, or ADGN-100 peptide) associated with one or more RNAi (e.g., siRNA). In some embodiments, the association is non-covalent. In some embodiments, the association is covalent.

In some embodiments, at least some of the cell-penetrating peptides in the RNAi delivery complex are linked to a targeting moiety. In some embodiments, the linkage is covalent. In some embodiments, the covalent linkage is by chemical coupling. In some embodiments, the covalent linkage is by genetic methods. In some embodiments, the molar ratio of cell-penetrating peptide to at least one of the one or more RNAi is between about 1:1 and about 100:1, or between about 1:1 and about 50:1, or about 20:1. In some embodiments, the CPP includes, but is not limited to, a PTD-based peptide, an amphipathic peptide, a poly-arginine-based peptide, an MPG peptide, a CADY peptide, a VEPEP peptide (such as a VEPEP-3, VEPEP-6, or VEPEP-9 peptide), an ADGN-100 peptide, a Pep-1 peptide, and a Pep-2 peptide.

In some embodiments, the RNAi delivery complex comprises an RNAi (such as an siRNA) targeting an endogenous gene. In some embodiments, the endogenous gene is involved in a disease or a condition. In some embodiments, the therapeutic RNAi targets a disease-associated form of the endogenous gene (e.g., a gene encoding a mutant protein, or a gene resulting in abnormal expression of a protein). In some embodiments, the RNAi targets an exogenous gene.

In some embodiments, the RNAi delivery complex comprises an RNAi (such as an siRNA) targeting KRAS. In some embodiments, the RNAi (e.g., siRNA) targets a mutant form of KRAS. In some embodiments, the RNAi (e.g., siRNA) specifically targets a mutant form of KRAS but not the wildtype form of KRAS. In some embodiments, the mutant form comprises an aberration of KRAS, wherein the aberration of KRAS comprises a mutation on codon 12, 13, 17, 34 or 61 of KRAS. In some embodiments, the mutant form comprises an aberration of KRAS, wherein the aberration of KRAS is selected from the group consisting of G12C, G12S, G12R, G12F, G12L, G12N, G12A, G12D, G12S, G12V, G13C, G13S, G13R, G13A, G13D, G13V, G13P, S17G, P34S, Q61E, Q61K, Q61L, Q61R, Q61P, Q61H, K117N, A146P, A146T and A146V. In some embodiments, the mutant form comprises an aberration of KRAS, wherein the aberration of KRAS is selected from the group consisting of G12C, G12S, G12R, G12F, G12L, G12N, G12A, G12D, G12V, G13C, G13S, G13D, G13V, G13P, S17G, P34S, Q61K, Q61L, Q61R, and Q61H. In some embodiments, the mutant form comprises an aberration of KRAS, wherein the aberration of KRAS is selected from the group consisting of G12C, G12R, G12S, G12A, G12D, G12V, G13C, G13R, G13S, G13A, G13D, G13V, Q61K, Q61L, Q61R, Q61H, K117N. A146P, A146T and A146V. In some embodiments, the mutant form comprises an aberration of KRAS, wherein the aberration of KRAS is selected from the group consisting of KRAS G12A, G12C, G12D, G12R, G12S, G12V, G13A, G13C, G13D, G13R, G13S, G13V, Q61E, Q61H, Q61K, Q61L, Q61P, and Q61R. In some embodiments, the aberration of KRAS is selected from the group consisting of KRAS G12C, G12D, G12R, G12S, G12V and G13D. In some embodiments, the aberration of KRAS comprises G12C. In some embodiments, the aberration of KRAS comprises G12D. In some embodiments, the aberration of KRAS comprises Q61K. In some embodiments, the aberration of KRAS comprises G12C and G12D. In some embodiments, the aberration of KRAS comprises G12C and Q61K. In some embodiments, the aberration of KRAS comprises G12D and Q61K. In some embodiments, the aberration of KRAS comprises G12C, G12D and Q61K.

In some embodiments, the RNAi delivery complex comprises an RNAi (such as an siRNA) targeting a plurality of mutant forms of KRAS. In some embodiments, the plurality of mutant forms comprises a plurality of aberrations of KRAS, wherein the plurality of aberrations of KRAS comprise at least two or more mutations on codon 12, 13, 17, 34 and/or 61 of KRAS. In some embodiments, the plurality of aberrations of KRAS comprises at least two or more mutations on codon 12 and 61 of KRAS. In some embodiments, the aberration of KRAS is selected from the group consisting of G12C, G12S, G12R, G12F, G12L, G12N, G12A, G12D, G12S, G12V, G13C, G13S, G13R, G13A, G13D, G13V, G13P, S17G, P34S, Q61E, Q61K, Q61L, Q61R, Q61P, Q61H, K17N, A146P, A146T and A146V. In some embodiments, the aberrations of KRAS are selected from the group consisting of G12C, G12S, G12R, G12F, G12L, G12N, G12A, G12D, G12V, G13C, G13S, G13D, G13V, G13P, S17G, P34S, Q61K, Q61L, Q61R, and Q61H. In some embodiments, the aberrations of KRAS are selected from the group consisting of G12C, G12R, G12S, G12A, G12D, G12V, G13C, G13R, G13S, G13A, G13D, G13V, Q61K, Q61L, Q61R, Q61H, K117N, A146P, A146T and A146V. In some embodiments, the aberrations of KRAS is selected from the group consisting of KRAS G12A, G12C, G12D, G12R, G12S, G12V, G13A, G13C, G13D, G13R, G13S, G13V, Q61E, Q61H, Q61K, Q61L, Q61P, and Q61R. In some embodiments, the aberrations of KRAS are selected from the group consisting of KRAS G12C, G12D, G12R, G12S, G12V and G13D. In some embodiments, the aberrations of KRAS are selected from the group consisting of KRAS G2C, G12D, and Q61K. In some embodiments, the aberrations of KRAS comprise G12C and G12D. In some embodiments, the aberrations of KRAS comprise G12C and Q61K. In some embodiments, the aberrations of KRAS comprise G12D and Q61K. In some embodiments, the aberration of KRAS comprises G12C, G12D and Q61K.

In some embodiments, the RNAi delivery complex comprises a plurality of RNAi (e.g., siRNA) comprising a first RNAi (e.g., a first siRNA) and a second RNAi (e.g., a second siRNA), wherein the first RNAi targets a first mutant form of KRAS, and wherein the second RNAi targets a second mutant form of KRAS. In some embodiments, the first RNAi and/or the second RNAi do not target the wildtype form of KRAS. In some embodiments, the first mutant form and/or the second mutant form comprises an aberration of KRAS, wherein the aberration of KRAS comprises a mutation on codon 12, 13, 17, 34 and/or 61 of KRAS. In some embodiments, the first mutant form and/or the second mutant form comprises an aberration of KRAS, wherein the aberration of KRAS comprises a mutation on codon 12 or 61 of KRAS. In some embodiments, the first mutant form comprises an aberration of KRAS comprising a mutation on codon 12, and the second mutant form comprises an aberration of KRAS comprising a mutation on codon 61. In some embodiments, the first mutant form and/or the second mutant form comprises an aberration of KRAS, wherein the aberration of KRAS is selected from the group consisting of G12C, G12S, G12R, G12F, G12L, G12N, G12A, G12D, G12S, G12V, G13C, G13S, G13R, G13A, G13D, G13V, G13P, S17G, P34S, Q61E, Q61K, Q61L, Q61R, Q61P, Q61H, K117N, A146P, A146T and A146V. In some embodiments, the first mutant form and/or the second mutant form comprises an aberration of KRAS, wherein the aberration of KRAS is selected from the group consisting of G12C, G12S, G12R, G12F, G12L, G12N, G12A, G12D, G12V, G13C, G13S, G13D, G3V, G13P, S17G, P34S, Q61K, Q61L, Q61R, and Q61H. In some embodiments, the first mutant form and/or the second mutant form comprises an aberration of KRAS, wherein the aberration of KRAS is selected from the group consisting of G12C, G12R, G12S, G12A, G12D, G12V, G13C, G13R, G13S, G13A, G13D, G13V, Q61K. Q61L, Q61R, Q61H, K117N, A146P, A146T and A146V. In some embodiments, the first mutant form and/or the second mutant form comprises an aberration of KRAS, wherein the aberration of KRAS is selected from the group consisting of KRAS G12A, G12C, G12D, G12R, G12S, G12V, G13A, G13C, G13D, G13R, G13S, G13V, Q61E, Q61H, Q61K, Q61L, Q61P, and Q61R. In some embodiments, the first mutant form and/or the second mutant form comprises an aberration of KRAS, wherein the aberration of KRAS is selected from the group consisting of KRAS G12C, G12D, G12R, G12S, G12V and G13D. In some embodiments, the first mutant form and/or the second mutant form comprises an aberration of KRAS, wherein the aberration of KRAS is selected from G12C, G12D and Q61K. In some embodiments, the first mutant form comprises an aberration of KRAS comprising KRAS G12C, and the second mutant form comprises an aberration of KRAS comprising KRAS G12D. In some embodiments, the first mutant form comprises an aberration of KRAS comprising KRAS G12C, and the second mutant form comprises an aberration of KRAS comprising KRAS Q61K. In some embodiments, the first mutant form comprises an aberration of KRAS comprising KRAS G12D, and the second mutant form comprises an aberration of KRAS comprising KRAS Q61K.

In some embodiments, the RNAi delivery complex comprises a plurality of RNAi (e.g., siRNA) comprising a first RNAi (e.g., a first siRNA), a second RNAi (e.g., a second siRNA), and a third RNAi (e.g., siRNA). In some embodiments, the first RNAi targets a first mutant form of KRAS, the second RNAi targets a second mutant form of KRAS, and the third RNAi targets a third mutant form of KRAS. In some embodiments, the first, second and third KRAS mutant form each comprises an aberration of KRAS comprising a mutation on codon 12, 13, 17, 34 and/or 61 of KRAS. In some embodiments, the first, second and third KRAS mutant form each comprises an aberration of KRAS selected from the group consisting of G12C, G12S, G12R, G12F, G12L, G12N, G12A, G12D, G12S, G12V, G13C, G13S, G13R, G13A, G13D, G13V, G13P, S17G, P34S, Q61E, Q61K, Q61L, Q616R, Q61P, Q61H, K117N, A146P, A146T and A146V. In some embodiments, the first, second and third KRAS mutant form each comprises an aberration of KRAS selected from the group consisting of G12C, G12S, G12R, G12F, G12L. G12N, G12A, G12D, G12V, G13C, G13S, G13D, G13V, G13P, S17G, P34S, Q61K, Q61L, Q61R, and Q61H. In some embodiments, the first, second and third KRAS mutant form each comprises an aberration of KRAS selected from the group consisting of G12C, G12R, G12S, G12A, G12D, G12V, G13C, G13R, G13S, G3A, G13D, G3V, Q61K, Q61L, Q61R, Q61H, K117N, A146P, A146T and A146V. In some embodiments, the first, second and third KRAS mutant form each comprises an aberration of KRAS selected from the group consisting of KRAS G12A, G12C, G12D, G12R, G12S, G12V, G13A, G13C, G13D, G13R, G13S, G13V, Q61E, Q61H, Q61K. Q61L, Q61P, and Q61R. In some embodiments, the first, second and third KRAS mutant form each comprises an aberration of KRAS selected from the group consisting of KRAS G12C, G12D, G12R, G12S, G12V, G13D and Q61K. In some embodiments, the first, second and third KRAS mutant form each comprises an aberration of KRAS selected from the group consisting of G12C, G12D and Q61K. In some embodiments, the first mutant form comprises an aberration of KRAS comprising KRAS G12C, the second mutant form comprises an aberration of KRAS comprising KRAS G12D, and the third mutant form comprises an aberration of KRAS comprising KRAS Q61K.

In some embodiments, the RNAi (e.g., siRNA) comprises an RNAi (e.g., siRNA) targeting KRAS comprising a sequence of 5′-GUUGGAGCUUGUGGCGUAGTT-3′ (sense) (SEQ ID NO: 83), 5′-CUACGCCACCAGCUCCAACTT-3 (anti-sense) (SEQ ID NO: 84), 5′-GAAGUGCAUACACCGAGACTT-3′ (sense) (SEQ ID NO: 86), 5-GUCUCGGUGUAGCACUUCTT-3′ (anti-sense) (SEQ ID NO: 87), 5′-GUUGGAGCUGUUGGCGUAGTT-3′ (sense) (SEQ ID NO: 88) and/or 5′-CUACGCCAACAGCUCCAACTT-3′ (anti-sense) (SEQ ID NO: 89). In some embodiments, the RNAi (e.g., siRNA) comprises an RNAi (e.g., siRNA) targeting KRAS comprising a nucleic acid sequence selected from sequences with SEQ ID NOS: 83, 84, 86-89. In some embodiments, the RNAi (e.g., siRNA) comprises an RNAi (e.g., siRNA) targeting KRAS comprising a sequence targeting KRAS G12S, such as the siRNA sequences disclosed in Acunzo, M. et al., Proc Natl Acad Sci USA. 2017 May 23; 114(21):E4203-E4212. In some embodiments, the RNAi (e.g., siRNA) comprises an RNAi (e.g., siRNA) targeting KRAS as disclosed in WO2014013995, JP2013212052, WO2014118817, WO2012129352, WO2017179660, JP2013544505, U.S. Pat. Nos. 8,008,474, 7,745,611, 7,576,197, 7,507,811, each of which is incorporated fully in this application.

In some embodiments, the mRNA delivery complex described herein further comprises an RNAi (such as siRNA), or is to be administered in combination with an RNAi as described above. In some embodiments, the complex and/or nanoparticle comprises a first mRNA encoding a first protein, and a second mRNA encoding a second protein. In some embodiments, the complex and/or nanoparticle further comprises a first RNAi (e.g., siRNA) targeting a first endogenous gene and a second RNAi (e.g., siRNA) targeting a second endogenous gene, or is to be administered in combination with the first and second RNAi. In some embodiments, the complex and/or nanoparticle further comprises a first RNAi (e.g., siRNA) targeting a first mutant form of an oncogen and a second RNAi (e.g., siRNA) targeting a second mutant form of the oncogene, or is to be administered in combination with the first and second RNAi. In some embodiments, the complex and/or nanoparticle comprises an mRNA encoding a protein, such as a therapeutic protein, and an RNAi (e.g., siRNA) targeting an endogenous gene. In some embodiments, the RNAi is a therapeutic RNAi targeting an endogenous gene involved in a disease or condition. In some embodiments, the therapeutic RNAi targets a disease-associated form of the endogenous gene (e.g., a gene encoding a mutant protein, or a gene resulting in abnormal expression of a protein). In some embodiments, the complex and/or nanoparticle comprises an mRNA and an RNAi, wherein the mRNA and RNAi are both useful for treating the same disease or condition. In some embodiments, the mRNA alone and/or the RNAi alone are ineffective for treating the disease or condition, but when used in combination are effective for treating the disease or condition. In some embodiments, the mRNA encodes a tumor suppressor protein involved in a cancer, and the RNAi targets an oncogene involved in the cancer.

CPPs can be covalently associated to mRNA using chemical conjugation. For example, CPPs can be linked to mRNA via cross linking involving either C-terminal cysteamide/cysteine or an N-terminal beta-Alanine bridge. mRNA can also be covalently linked to various moieties inside a peptide chain using any technique known in the art for such purposes, including for example chemistry such as 6-maleimidohexanoic acid N-hydroxysuccinimide ester.

In some embodiments, there is provided an mRNA delivery complex for intracellular delivery of an mRNA comprising a cell-penetrating peptide associated with the mRNA, wherein the cell-penetrating peptide comprises the amino acid sequence of a PEP-1 peptide, a PEP-2 peptide, a VEPEP-3 peptide, a VEPEP-6 peptide, a VEPEP-9 peptide, or an ADGN-100 peptide. In some embodiments, the PEP-1 peptide comprises the amino acid sequence of SEQ ID NO: 71. In some embodiments, the PEP-2 peptide comprises the amino acid sequence of SEQ ID NO: 72. In some embodiments, the PEP-3 peptide comprises the amino acid sequence of SEQ ID NO: 73. In some embodiments, the VEPEP-3 peptide comprises the amino acid sequence of any one of SEQ ID NOs: 1-14, 75, and 76. In some embodiments, the VEPEP-6 peptide comprises the amino acid sequence of any one of SEQ ID NOs: 15-40, and 77. In some embodiments, the VEPEP-9 peptide comprises the amino acid sequence of any one of SEQ ID NOs: 41-52, and 78. In some embodiments, the ADGN-100 peptide comprises the amino acid sequence of any one of SEQ ID NOs: 53-70, 79, and 80. In some embodiments, the mRNA delivery complex further comprises an RNAi, or is to be administered in combination with an RNAi.

In some embodiments, there is provided an mRNA delivery complex comprising a cell-penetrating peptide and a plurality of mRNA, wherein each of the plurality of mRNA encodes a different protein, and wherein the cell-penetrating peptide comprises the amino acid sequence of a PEP-1 peptide, a PEP-2 peptide, a VEPEP-3 peptide, a VEPEP-6 peptide, a VEPEP-9 peptide, or an ADGN-100 peptide. In some embodiments, the PEP-1 peptide comprises the amino acid sequence of SEQ ID NO: 71. In some embodiments, the PEP-2 peptide comprises the amino acid sequence of SEQ ID NO: 72. In some embodiments, the PEP-3 peptide comprises the amino acid sequence of SEQ ID NO: 73. In some embodiments, the VEPEP-3 peptide comprises the amino acid sequence of any one of SEQ ID NOs: 1-14, 75, and 76. In some embodiments, the VEPEP-6 peptide comprises the amino acid sequence of any one of SEQ ID NOs: 15-40, and 77. In some embodiments, the VEPEP-9 peptide comprises the amino acid sequence of any one of SEQ ID NOs: 41-52, and 78. In some embodiments, the ADGN-100 peptide comprises the amino acid sequence of any one of SEQ ID NOs: 53-70, 79, and 80. In some embodiments, the mRNA delivery complex further comprises an RNAi, or is to be administered in combination with an RNAi.

In some embodiments, there is provided an mRNA delivery complex for intracellular delivery of an mRNA comprising a cell-penetrating peptide associated with the mRNA, wherein the mRNA encodes a tumor suppressor protein corresponding to a tumor suppressor gene. In some embodiments, the cell-penetrating peptide comprises (or consists of) the amino acid sequence of a PEP-1 peptide, a PEP-2 peptide, a VEPEP-3 peptide, a VEPEP-6 peptide, a VEPEP-9 peptide, or an ADGN-100 peptide. In some embodiments, the PEP-1 peptide comprises the amino acid sequence of SEQ ID NO: 71. In some embodiments, the PEP-2 peptide comprises the amino acid sequence of SEQ ID NO: 72. In some embodiments, the PEP-3 peptide comprises the amino acid sequence of SEQ ID NO: 73. In some embodiments, the VEPEP-3 peptide comprises the amino acid sequence of any one of SEQ ID NOs: 1-14, 75, and 76. In some embodiments, the VEPEP-6 peptide comprises the amino acid sequence of any one of SEQ ID NOs: 15-40, and 77. In some embodiments, the VEPEP-9 peptide comprises the amino acid sequence of any one of SEQ ID NOs: 41-52, and 78. In some embodiments, the ADGN-100 peptide comprises the amino acid sequence of any one of SEQ ID NOs: 53-70, 79, and 80. In some embodiments, the tumor-suppressor protein is a Retinoblastoma protein (pRb). In some embodiments, the tumor-suppressor protein is a p53 tumor-suppressor protein. In some embodiments, the corresponding tumor-suppressor gene is Phosphatase and tensin homolog (PTEN). In some embodiments, the corresponding tumor-suppressor gene is PTEN, Retinoblastoma RB (or RB1), TP53, CDKN2A (INK4A), MLH1, MSH2, MSH6, WT1, WT2, NF1, NF2N, VHL, KLF4, pVHL, APC, CD95, ST5, YPEL3, ST7, APC, MADR2, BRCA1, BRCA2, Patched. TSC1, TSC2, PALB2, or ST14.

In some embodiments, there is provided an mRNA delivery complex for intracellular delivery of an mRNA comprising a cell-penetrating peptide associated with the mRNA, wherein the mRNA encodes a protein, and wherein the deficiency of the protein results in a disease or disorder. In some embodiments, the cell-penetrating peptide comprises (or consists of) the amino acid sequence of a PEP-1 peptide, a PEP-2 peptide, a VEPEP-3 peptide, a VEPEP-6 peptide, a VEPEP-9 peptide, or an ADGN-100 peptide. In some embodiments, the PEP-1 peptide comprises the amino acid sequence of SEQ ID NO: 71. In some embodiments, the PEP-2 peptide comprises the amino acid sequence of SEQ ID NO: 72. In some embodiments, the PEP-3 peptide comprises the amino acid sequence of SEQ ID NO: 73. In some embodiments, the VEPEP-3 peptide comprises the amino acid sequence of any one of SEQ ID NOs: 1-14, 75, and 76. In some embodiments, the VEPEP-6 peptide comprises the amino acid sequence of any one of SEQ ID NOs: 15-40, and 77. In some embodiments, the VEPEP-9 peptide comprises the amino acid sequence of any one of SEQ ID NOs: 41-52, and 78. In some embodiments, the ADGN-100 peptide comprises the amino acid sequence of any one of SEQ ID NOs: 53-70, 79, and 80. In some embodiments, the protein is Frataxin. In some embodiments, the protein is alpha 1 antitrypsin. In some embodiments, the protein is factor VIII. In some embodiments, the protein is factor IX.

In some embodiments, there is provided an mRNA delivery complex for intracellular delivery of an mRNA comprising a cell-penetrating peptide associated with the mRNA, wherein the mRNA encodes a protein, and wherein expression of the protein in an individual modulates an immune response to the protein in the individual. In some embodiments, the cell-penetrating peptide comprises (or consists of) the amino acid sequence of a PEP-1 peptide, a PEP-2 peptide, a VEPEP-3 peptide, a VEPEP-6 peptide, a VEPEP-9 peptide, or an ADGN-100 peptide. In some embodiments, the PEP-1 peptide comprises the amino acid sequence of SEQ ID NO: 71. In some embodiments, the PEP-2 peptide comprises the amino acid sequence of SEQ ID NO: 72. In some embodiments, the PEP-3 peptide comprises the amino acid sequence of SEQ ID NO: 73. In some embodiments, the VEPEP-3 peptide comprises the amino acid sequence of any one of SEQ ID NOs: 1-14, 75, and 76. In some embodiments, the VEPEP-6 peptide comprises the amino acid sequence of any one of SEQ ID NOs: 15-40, and 77. In some embodiments, the VEPEP-9 peptide comprises the amino acid sequence of any one of SEQ ID NOs: 41-52, and 78. In some embodiments, the ADGN-100 peptide comprises the amino acid sequence of any one of SEQ ID NOs: 53-70, 79, and 80. In some embodiments, the protein is an antigen. In some embodiments, the antigen is a disease-associated antigen (e.g., a tumor-associated antigen), and expression of the antigen in the individual results in an increased immune response to the antigen in the individual. In some embodiments, the antigen is a self-antigen, and expression of the antigen in the individual results in a decreased immune response to the antigen in the individual.

In some embodiments, there is provided an mRNA delivery complex for intracellular delivery of an mRNA comprising a cell-penetrating peptide associated with the mRNA, wherein the mRNA encodes an antibody or antigen-binding fragment thereof. In some embodiments, the cell-penetrating peptide comprises (or consists of) the amino acid sequence of a PEP-1 peptide, a PEP-2 peptide, a VEPEP-3 peptide, a VEPEP-6 peptide, a VEPEP-9 peptide, or an ADGN-100 peptide. In some embodiments, the PEP-1 peptide comprises the amino acid sequence of SEQ ID NO: 71. In some embodiments, the PEP-2 peptide comprises the amino acid sequence of SEQ ID NO: 72. In some embodiments, the PEP-3 peptide comprises the amino acid sequence of SEQ ID NO: 73. In some embodiments, the VEPEP-3 peptide comprises the amino acid sequence of any one of SEQ ID NOs: 1-14, 75, and 76. In some embodiments, the VEPEP-6 peptide comprises the amino acid sequence of any one of SEQ ID NOs: 15-40, and 77. In some embodiments, the VEPEP-9 peptide comprises the amino acid sequence of any one of SEQ ID NOs: 41-52, and 78. In some embodiments, the ADGN-100 peptide comprises the amino acid sequence of any one of SEQ ID NOs: 53-70, 79, and 80. In some embodiments, the antibody is a therapeutic antibody. In some embodiments, the antibody is a bispecific antibody, such as a bispecific T cell engager (BiTE). In some embodiments, the antibody specifically binds to a disease-associated antigen, such as a tumor-associated antigen.

In some embodiments, there is provided an mRNA delivery complex for intracellular delivery of an mRNA comprising a cell-penetrating peptide associated with the mRNA, wherein the mRNA comprises a reporter mRNA. In some embodiments, the cell-penetrating peptide comprises (or consists of) the amino acid sequence of a PEP-1 peptide, a PEP-2 peptide, a VEPEP-3 peptide, a VEPEP-6 peptide, a VEPEP-9 peptide, or an ADGN-100 peptide. In some embodiments, the PEP-1 peptide comprises the amino acid sequence of SEQ ID NO: 71. In some embodiments, the PEP-2 peptide comprises the amino acid sequence of SEQ ID NO: 72. In some embodiments, the PEP-3 peptide comprises the amino acid sequence of SEQ ID NO: 73. In some embodiments, the VEPEP-3 peptide comprises the amino acid sequence of any one of SEQ ID NOs: 1-14, 75, and 76. In some embodiments, the VEPEP-6 peptide comprises the amino acid sequence of any one of SEQ ID NOs: 15-40, and 77. In some embodiments, the VEPEP-9 peptide comprises the amino acid sequence of any one of SEQ ID NOs: 41-52, and 78. In some embodiments, the ADGN-100 peptide comprises the amino acid sequence of any one of SEQ ID NOs: 53-70, 79, and 80. In some embodiments, the mRNA comprises a EGFP mRNA, for example, CleanCap EGFP mRNA, CleanCap EGFP mRNA (5moU), or CleanCap Cyanine 5 EGFP mRNA (5moU). In some embodiments, the mRNA comprises a Luc mRNA, for example, CleanCap Fluc mRNA, CleanCap Fluc mRNA (5moU), CleanCap Cyanine 5 Fluc mRNA (5moU), CleanCap Gaussia Luc mRNA (5moU), or CleanCap Renilla Luc mRNA (5moU). In some embodiments, the mRNA comprises an mRNA selected from CleanCap β-gal mRNA, CleanCap β-gal mRNA (5moU) and CleanCap mCherry mRNA (5mOU).

In some embodiments, there is provided an mRNA delivery complex for intracellular delivery of an mRNA comprising a cell-penetrating peptide associated with the mRNA, wherein the cell-penetrating peptide comprises the amino acid sequence of a PEP-1 peptide, a PEP-2 peptide, a VEPEP-3 peptide, a VEPEP-6 peptide, a VEPEP-9 peptide, or an ADGN-100 peptide, and wherein the mRNA encodes a tumor suppressor protein corresponding to a tumor suppressor gene. In some embodiments, the PEP-1 peptide comprises the amino acid sequence of SEQ ID NO: 71. In some embodiments, the PEP-2 peptide comprises the amino acid sequence of SEQ ID NO: 72. In some embodiments, the PEP-3 peptide comprises the amino acid sequence of SEQ ID NO: 73. In some embodiments, the VEPEP-3 peptide comprises the amino acid sequence of any one of SEQ ID NOs: 1-14, 75, and 76. In some embodiments, the VEPEP-6 peptide comprises the amino acid sequence of any one of SEQ ID NOs: 15-40, and 77. In some embodiments, the VEPEP-9 peptide comprises the amino acid sequence of any one of SEQ ID NOs: 41-52, and 78. In some embodiments, the ADGN-100 peptide comprises the amino acid sequence of any one of SEQ ID NOs: 53-70, 79, and 80. In some embodiments, the tumor-suppressor protein is a Retinoblastoma protein (pRb). In some embodiments, the tumor-suppressor protein is a p53 tumor-suppressor protein. In some embodiments, the corresponding tumor-suppressor gene is Phosphatase and tensin homolog (PTEN). In some embodiments, the corresponding tumor-suppressor gene is PTEN, Retinoblastoma RB (or RB1), TP53, CDKN2A (INK4A), MLH1, MSH2, MSH6, WT1, WT2, NF1, NF2N, VHL, KLF4, pVHL, APC, CD95, ST5, YPEL3, ST7, APC, MADR2, BRCA1, BRCA2, Patched, TSC1, TSC2, PALB2, or ST14.

In some embodiments, there is provided an mRNA delivery complex for intracellular delivery of an mRNA comprising a cell-penetrating peptide associated with the mRNA, wherein the cell-penetrating peptide comprises the amino acid sequence of a PEP-1 peptide, a PEP-2 peptide, a VEPEP-3 peptide, a VEPEP-6 peptide, a VEPEP-9 peptide, or an ADGN-100 peptide, and wherein the mRNA encodes a protein, wherein the deficiency of the protein results in a disease or disorder. In some embodiments, the PEP-1 peptide comprises the amino acid sequence of SEQ ID NO: 71. In some embodiments, the PEP-2 peptide comprises the amino acid sequence of SEQ ID NO: 72. In some embodiments, the PEP-3 peptide comprises the amino acid sequence of SEQ ID NO: 73. In some embodiments, the VEPEP-3 peptide comprises the amino acid sequence of any one of SEQ ID NOs: 1-14, 75, and 76. In some embodiments, the VEPEP-6 peptide comprises the amino acid sequence of any one of SEQ ID NOs: 15-40, and 77. In some embodiments, the VEPEP-9 peptide comprises the amino acid sequence of any one of SEQ ID NOs: 41-52, and 78. In some embodiments, the ADGN-100 peptide comprises the amino acid sequence of any one of SEQ ID NOs: 53-70, 79, and 80. In some embodiments, the protein is Frataxin. In some embodiments, the protein is alpha 1 antitrypsin. In some embodiments, the protein is factor VIII. In some embodiments, the protein is factor IX.

In some embodiments, there is provided an mRNA delivery complex for intracellular delivery of an mRNA comprising a cell-penetrating peptide associated with the mRNA, wherein the cell-penetrating peptide comprises the amino acid sequence of a PEP-1 peptide, a PEP-2 peptide, a VEPEP-3 peptide, a VEPEP-6 peptide, a VEPEP-9 peptide, or an ADGN-100 peptide, and wherein the mRNA encodes a protein, wherein expression of the protein in an individual modulates an immune response to the protein in the individual. In some embodiments, the PEP-1 peptide comprises the amino acid sequence of SEQ ID NO: 71. In some embodiments, the PEP-2 peptide comprises the amino acid sequence of SEQ ID NO: 72. In some embodiments, the PEP-3 peptide comprises the amino acid sequence of SEQ ID NO: 73. In some embodiments, the VEPEP-3 peptide comprises the amino acid sequence of any one of SEQ ID NOs: 1-14, 75, and 76. In some embodiments, the VEPEP-6 peptide comprises the amino acid sequence of any one of SEQ ID NOs: 15-40, and 77. In some embodiments, the VEPEP-9 peptide comprises the amino acid sequence of any one of SEQ ID NOs: 41-52, and 78. In some embodiments, the ADGN-100 peptide comprises the amino acid sequence of any one of SEQ ID NOs: 53-70, 79, and 80. In some embodiments, the protein is an antigen. In some embodiments, the antigen is a disease-associated antigen (e.g., a tumor-associated antigen), and expression of the antigen in the individual results in an increased immune response to the antigen in the individual. In some embodiments, the antigen is a self-antigen, and expression of the antigen in the individual results in a decreased immune response to the antigen in the individual.

In some embodiments, there is provided an mRNA delivery complex for intracellular delivery of an mRNA comprising a cell-penetrating peptide associated with the mRNA, wherein the cell-penetrating peptide comprises the amino acid sequence of a PEP-1 peptide, a PEP-2 peptide, a VEPEP-3 peptide, a VEPEP-6 peptide, a VEPEP-9 peptide, or an ADGN-100 peptide, and wherein the mRNA encodes an antibody or antigen-binding fragment thereof. In some embodiments, the PEP-1 peptide comprises the amino acid sequence of SEQ ID NO: 71. In some embodiments, the PEP-2 peptide comprises the amino acid sequence of SEQ ID NO: 72. In some embodiments, the PEP-3 peptide comprises the amino acid sequence of SEQ ID NO: 73. In some embodiments, the VEPEP-3 peptide comprises the amino acid sequence of any one of SEQ ID NOs: 1-14, 75, and 76. In some embodiments, the VEPEP-6 peptide comprises the amino acid sequence of any one of SEQ ID NOs: 15-40, and 77. In some embodiments, the VEPEP-9 peptide comprises the amino acid sequence of any one of SEQ ID NOs: 41-52, and 78. In some embodiments, the ADGN-100 peptide comprises the amino acid sequence of any one of SEQ ID NOs: 53-70, 79, and 80. In some embodiments, the antibody is a therapeutic antibody. In some embodiments, the antibody is a bispecific antibody, such as a bispecific T cell engager (BiTE). In some embodiments, the antibody specifically binds to a disease-associated antigen, such as a tumor-associated antigen.

In some embodiments, there is provided an mRNA delivery complex for intracellular delivery of an mRNA comprising a cell-penetrating peptide associated with the mRNA, wherein the cell-penetrating peptide comprises the amino acid sequence of a PEP-1 peptide, a PEP-2 peptide, a VEPEP-3 peptide, a VEPEP-6 peptide, a VEPEP-9 peptide, or an ADGN-100 peptide, and wherein the mRNA comprises a reporter mRNA. In some embodiments, the PEP-1 peptide comprises the amino acid sequence of SEQ ID NO: 71. In some embodiments, the PEP-2 peptide comprises the amino acid sequence of SEQ ID NO: 72. In some embodiments, the PEP-3 peptide comprises the amino acid sequence of SEQ ID NO: 73. In some embodiments, the VEPEP-3 peptide comprises the amino acid sequence of any one of SEQ ID NOs: 1-14, 75, and 76. In some embodiments, the VEPEP-6 peptide comprises the amino acid sequence of any one of SEQ ID NOs: 15-40, and 77. In some embodiments, the VEPEP-9 peptide comprises the amino acid sequence of any one of SEQ ID NOs: 41-52, and 78. In some embodiments, the ADGN-100 peptide comprises the amino acid sequence of any one of SEQ ID NOs: 53-70, 79, and 80. In some embodiments, the mRNA comprises a EGFP mRNA, for example, CleanCap EGFP mRNA, CleanCap EGFP mRNA (5moU), or CleanCap Cyanine 5 EGFP mRNA (5moU). In some embodiments, the mRNA comprises a Luc mRNA, for example, CleanCap Fluc mRNA, CleanCap Fluc mRNA (5moU), CleanCap Cyanine 5 Fluc mRNA (5moU), CleanCap Gaussia Luc mRNA (5moU), or CleanCap Renilla Luc mRNA (5moU). In some embodiments, the mRNA comprises an mRNA selected from CleanCap β-gal mRNA, CleanCap β-gal mRNA (5moU) and CleanCap mCherry mRNA (5mOU).

In some embodiments, an mRNA delivery complex according to any of the embodiments described herein further comprises an RNAi. In some embodiments, the RNAi comprises an siRNA. In some embodiments, the RNAi comprises a microRNA. In some embodiments, the RNAi targets an oncogene. In some embodiments, the oncogene is Smoothened. In some embodiments, the oncogene is rasK. In some embodiments, the oncogene is KRAS.

In some embodiments, an mRNA delivery complex according to any of the embodiments described herein is for administration in combination with an RNAi. In some embodiments, the RNAi is in a complex or nanoparticle comprising cell-penetrating peptides for delivering the RNAi into a cell. In some embodiments, the RNAi comprises an siRNA. In some embodiments, the RNAi comprises a microRNA. In some embodiments, the RNAi targets an oncogene. In some embodiments, the oncogene is Smoothened. In some embodiments, the oncogene is rasK. In some embodiments, the oncogene is KRAS.

In some embodiments, the mean size (diameter) of an mRNA delivery complex described herein is between any of about 20 nm and about 10 microns, including for example between about 30 nm and about 1 micron, between about 50 nm and about 750 nm, between about 100 nm and about 500 nm, between 100 nm and 250 nm, and between about 200 nm and about 400 nm. In some embodiments, the mRNA delivery complex is substantially non-toxic.

In some embodiments, the targeting moiety of an mRNA delivery complex described herein targets the mRNA delivery complex to a tissue or a specific cell type. In some embodiments, the tissue is a tissue in need of treatment. In some embodiments, the targeting moiety targets the mRNA delivery complex to a tissue or cell that can be treated by the mRNA.

### Nanoparticles Comprising Cell-Penetrating Peptides

In some embodiments, there is provided a nanoparticle for intracellular delivery of an mRNA comprising a core comprising one or more mRNA delivery complexes described herein. In some embodiments, the nanoparticle core comprises a plurality of mRNA delivery complexes. In some embodiments, the nanoparticle core comprises a plurality of mRNA delivery complexes present in a predetermined ratio. In some embodiments, the predetermined ratio is selected to allow the most effective use of the nanoparticle in any of the methods described below in more detail. In some embodiments, the nanoparticle core further comprises one or more additional cell-penetrating peptides and/or one or more additional mRNA. In some embodiments, the nanoparticle core further comprises one or more additional cell-penetrating peptides associated with (such as covalently or non-covalently) one or more additional mRNA. In some embodiments, the one or more additional cell-penetrating peptides include, but are not limited to, a PTD-based peptide, an amphipathic peptide, a poly-arginine-based peptide, an MPG peptide, a CADY peptide, a VEPEP peptide (such as a VEPEP-3, VEPEP-6, or VEPEP-9 peptide), an ADGN-100 peptide, a Pep-1 peptide, and a Pep-2 peptide. In some embodiments, at least some of the one or more additional cell-penetrating peptides are linked to a targeting moiety. In some embodiments, the linkage is covalent. In some embodiments, the covalent linkage is by chemical coupling. In some embodiments, the covalent linkage is by genetic methods.

In some embodiments, there is provided a nanoparticle for intracellular delivery of an mRNA comprising a core comprising one or more cell-penetrating peptides (e.g., a PEP-1, PEP-2, VEPEP-3, VEPEP-6, VEPEP-9, or ADGN-100 peptide) associated with the mRNA. In some embodiments, the association is non-covalent. In some embodiments, the association is covalent.

In some embodiments, the nanoparticle comprises an mRNA encoding a protein, such as a therapeutic protein. In some embodiments, the mRNA encodes a tumor suppressor protein. In some embodiments, the mRNA encodes a tumor suppressor protein, wherein the protein corresponds to a tumor suppressor gene. In some embodiments, the tumor-suppressor protein is a Retinoblastoma protein (pRb). In some embodiments, the tumor-suppressor protein is a p53 tumor-suppressor protein. In some embodiments, the corresponding tumor-suppressor gene is Phosphatase and tensin homolog (PTEN). In some embodiments, the corresponding tumor-suppressor gene is PTEN, Retinoblastoma RB (or RB1), TP53, CDKN2A (NK4A), MLH1, MSH2, MSH6, WT1, WT2, NF1, NF2N, VHL, KLF4, pVHL, APC, CD95, ST5, YPEL3, ST7, APC, MADR2, BRCA1, BRCA2, Patched, TSC1, TSC2, PALB2, or ST14.

In some embodiments, the nanoparticle comprises an mRNA, wherein the mRNA encodes a protein, wherein the deficiency of the protein results in a disease or disorder. In some embodiments, the protein is Frataxin. In some embodiments, the protein is factor VIII. In some embodiments, the protein is factor IX.

In some embodiments, the nanoparticle comprises an mRNA, wherein the mRNA contained in an mRNA delivery complex according to any of the embodiments described herein comprises a reporter mRNA. In some embodiments, the mRNA comprises a EGFP mRNA, for example, CleanCap EGFP mRNA, CleanCap EGFP mRNA (5moU), or CleanCap Cyanine 5 EGFP mRNA (5moU). In some embodiments, the mRNA comprises a Luc mRNA, for example, CleanCap Fluc mRNA, CleanCap Fluc mRNA (5moU), CleanCap Cyanine 5 Fluc mRNA (5moU), CleanCap Gaussia Luc mRNA (5moU), or CleanCap Renilla Luc mRNA (5moU). In some embodiments, the mRNA comprises an mRNA selected from CleanCap β-gal mRNA, CleanCap β-gal mRNA (5moU) and CleanCap mCherry mRNA (5mOU).

In some embodiments, there is provided an mRNA delivery nanoparticle for intracellular delivery of an mRNA comprising a cell-penetrating peptide associated with the mRNA, wherein the mRNA encodes a tumor suppressor protein corresponding to a tumor suppressor gene. In some embodiments, the cell-penetrating peptide comprises (or consists of) the amino acid sequence of a PEP-1 peptide, a PEP-2 peptide, a VEPEP-3 peptide, a VEPEP-6 peptide, a VEPEP-9 peptide, or an ADGN-100 peptide. In some embodiments, the PEP-1 peptide comprises the amino acid sequence of SEQ ID NO: 71. In some embodiments, the PEP-2 peptide comprises the amino acid sequence of SEQ ID NO: 72. In some embodiments, the PEP-3 peptide comprises the amino acid sequence of SEQ ID NO: 73. In some embodiments, the VEPEP-3 peptide comprises the amino acid sequence of any one of SEQ ID NOs: 1-14, 75, and 76. In some embodiments, the VEPEP-6 peptide comprises the amino acid sequence of any one of SEQ ID NOs: 15-40, and 77. In some embodiments, the VEPEP-9 peptide comprises the amino acid sequence of any one of SEQ ID NOs: 41-52, and 78. In some embodiments, the ADGN-100 peptide comprises the amino acid sequence of any one of SEQ ID NOs: 53-70, 79, and 80. In some embodiments, the tumor-suppressor protein is a Retinoblastoma protein (pRb). In some embodiments, the tumor-suppressor protein is a p53 tumor-suppressor protein. In some embodiments, the corresponding tumor-suppressor gene is Phosphatase and tensin homolog (PTEN). In some embodiments, the corresponding tumor-suppressor gene is PTEN, Retinoblastoma RB (or RB1), TP53, CDKN2A (INK4A), MLH1, MSH2, MSH6, WT1, WT2, NF1. NF2N, VHL, KLF4, pVHL, APC, CD95, ST5, YPEL3, ST7, APC, MADR2, BRCA1, BRCA2, Patched, TSC1, TSC2, PALB2, or ST14.

In some embodiments, there is provided an mRNA delivery nanoparticle for intracellular delivery of an mRNA comprising a cell-penetrating peptide associated with the mRNA, wherein the mRNA encodes a protein, and wherein the deficiency of the protein results in a disease or disorder. In some embodiments, the cell-penetrating peptide comprises (or consists of) the amino acid sequence of a PEP-1 peptide, a PEP-2 peptide, a VEPEP-3 peptide, a VEPEP-6 peptide, a VEPEP-9 peptide, or an ADGN-100 peptide. In some embodiments, the PEP-1 peptide comprises the amino acid sequence of SEQ ID NO: 71. In some embodiments, the PEP-2 peptide comprises the amino acid sequence of SEQ ID NO: 72. In some embodiments, the PEP-3 peptide comprises the amino acid sequence of SEQ ID NO: 73. In some embodiments, the VEPEP-3 peptide comprises the amino acid sequence of any one of SEQ ID NOs: 1-14, 75, and 76. In some embodiments, the VEPEP-6 peptide comprises the amino acid sequence of any one of SEQ ID NOs: 15-40, and 77. In some embodiments, the VEPEP-9 peptide comprises the amino acid sequence of any one of SEQ ID NOs: 41-52, and 78. In some embodiments, the ADGN-100 peptide comprises the amino acid sequence of any one of SEQ ID NOs: 53-70, 79, and 80. In some embodiments, the protein is Frataxin. In some embodiments, the protein is alpha 1 antitrypsin. In some embodiments, the protein is factor VIII. In some embodiments, the protein is factor IX.

In some embodiments, there is provided an mRNA delivery nanoparticle for intracellular delivery of an mRNA comprising a cell-penetrating peptide associated with the mRNA, wherein the mRNA encodes a protein, and wherein expression of the protein in an individual modulates an immune response to the protein in the individual. In some embodiments, the cell-penetrating peptide comprises (or consists of) the amino acid sequence of a PEP-1 peptide, a PEP-2 peptide, a VEPEP-3 peptide, a VEPEP-6 peptide, a VEPEP-9 peptide, or an ADGN-100 peptide. In some embodiments, the PEP-1 peptide comprises the amino acid sequence of SEQ ID NO: 71. In some embodiments, the PEP-2 peptide comprises the amino acid sequence of SEQ ID NO: 72. In some embodiments, the PEP-3 peptide comprises the amino acid sequence of SEQ ID NO: 73. In some embodiments, the VEPEP-3 peptide comprises the amino acid sequence of any one of SEQ ID NOs: 1-14, 75, and 76. In some embodiments, the VEPEP-6 peptide comprises the amino acid sequence of any one of SEQ ID NOs: 15-40, and 77. In some embodiments, the VEPEP-9 peptide comprises the amino acid sequence of any one of SEQ ID NOs: 41-52, and 78. In some embodiments, the ADGN-100 peptide comprises the amino acid sequence of any one of SEQ ID NOs: 53-70, 79, and 80. In some embodiments, the protein is an antigen. In some embodiments, the antigen is a disease-associated antigen (e.g., a tumor-associated antigen), and expression of the antigen in the individual results in an increased immune response to the antigen in the individual. In some embodiments, the antigen is a self-antigen, and expression of the antigen in the individual results in a decreased immune response to the antigen in the individual.

In some embodiments, there is provided an mRNA delivery nanoparticle for intracellular delivery of an mRNA comprising a cell-penetrating peptide associated with the mRNA, wherein the mRNA encodes an antibody or antigen-binding fragment thereof. In some embodiments, the cell-penetrating peptide comprises (or consists of) the amino acid sequence of a PEP-1 peptide, a PEP-2 peptide, a VEPEP-3 peptide, a VEPEP-6 peptide, a VEPEP-9 peptide, or an ADGN-100 peptide. In some embodiments, the PEP-1 peptide comprises the amino acid sequence of SEQ ID NO: 71. In some embodiments, the PEP-2 peptide comprises the amino acid sequence of SEQ ID NO: 72. In some embodiments, the PEP-3 peptide comprises the amino acid sequence of SEQ ID NO: 73. In some embodiments, the VEPEP-3 peptide comprises the amino acid sequence of any one of SEQ ID NOs: 1-14, 75, and 76. In some embodiments, the VEPEP-6 peptide comprises the amino acid sequence of any one of SEQ ID NOs: 15-40, and 77. In some embodiments, the VEPEP-9 peptide comprises the amino acid sequence of any one of SEQ ID NOs: 41-52, and 78. In some embodiments, the ADGN-100 peptide comprises the amino acid sequence of any one of SEQ ID NOs: 53-70, 79, and 80. In some embodiments, the antibody is a therapeutic antibody. In some embodiments, the antibody is a bispecific antibody, such as a bispecific T cell engager (BiTE). In some embodiments, the antibody specifically binds to a disease-associated antigen, such as a tumor-associated antigen.

In some embodiments, there is provided an mRNA delivery nanoparticle for intracellular delivery of an mRNA comprising a cell-penetrating peptide associated with the mRNA, wherein the mRNA comprises a reporter mRNA. In some embodiments, the cell-penetrating peptide comprises (or consists of) the amino acid sequence of a PEP-1 peptide, a PEP-2 peptide, a VEPEP-3 peptide, a VEPEP-6 peptide, a VEPEP-9 peptide, or an ADGN-100 peptide. In some embodiments, the PEP-1 peptide comprises the amino acid sequence of SEQ ID NO: 71. In some embodiments, the PEP-2 peptide comprises the amino acid sequence of SEQ ID NO: 72. In some embodiments, the PEP-3 peptide comprises the amino acid sequence of SEQ ID NO: 73. In some embodiments, the VEPEP-3 peptide comprises the amino acid sequence of any one of SEQ ID NOs: 1-14, 75, and 76. In some embodiments, the VEPEP-6 peptide comprises the amino acid sequence of any one of SEQ ID NOs: 15-40, and 77. In some embodiments, the VEPEP-9 peptide comprises the amino acid sequence of any one of SEQ ID NOs: 41-52, and 78. In some embodiments, the ADGN-100 peptide comprises the amino acid sequence of any one of SEQ ID NOs: 53-70, 79, and 80. In some embodiments, the mRNA comprises a EGFP mRNA, for example, CleanCap EGFP mRNA, CleanCap EGFP mRNA (5moU), or CleanCap Cyanine 5 EGFP mRNA (5moU). In some embodiments, the mRNA comprises a Luc mRNA, for example, CleanCap Fluc mRNA, CleanCap Fluc mRNA (5moU), CleanCap Cyanine 5 Fluc mRNA (5moU). CleanCap Gaussia Luc mRNA (5moU), or CleanCap Renilla Luc mRNA (5moU). In some embodiments, the mRNA comprises an mRNA selected from CleanCap β-gal mRNA, CleanCap β-gal mRNA (5moU) and CleanCap mCherry mRNA (5mOU).

In some embodiments, there is provided an mRNA delivery nanoparticle for intracellular delivery of an mRNA comprising a cell-penetrating peptide associated with the mRNA, wherein the cell-penetrating peptide comprises the amino acid sequence of a PEP-1 peptide, a PEP-2 peptide, a VEPEP-3 peptide, a VEPEP-6 peptide, a VEPEP-9 peptide, or an ADGN-100 peptide, and wherein the mRNA encodes a tumor suppressor protein corresponding to a tumor suppressor gene. In some embodiments, the PEP-1 peptide comprises the amino acid sequence of SEQ ID NO: 71. In some embodiments, the PEP-2 peptide comprises the amino acid sequence of SEQ ID NO: 72. In some embodiments, the PEP-3 peptide comprises the amino acid sequence of SEQ ID NO: 73. In some embodiments, the VEPEP-3 peptide comprises the amino acid sequence of any one of SEQ ID NOs: 1-14, 75, and 76. In some embodiments, the VEPEP-6 peptide comprises the amino acid sequence of any one of SEQ ID NOs: 15-40, and 77. In some embodiments, the VEPEP-9 peptide comprises the amino acid sequence of any one of SEQ ID NOs: 41-52, and 78. In some embodiments, the ADGN-100 peptide comprises the amino acid sequence of any one of SEQ ID NOs: 53-70, 79, and 80. In some embodiments, the tumor-suppressor protein is a Retinoblastoma protein (pRb). In some embodiments, the tumor-suppressor protein is a p53 tumor-suppressor protein. In some embodiments, the corresponding tumor-suppressor gene is Phosphatase and tensin homolog (PTEN). In some embodiments, the corresponding tumor-suppressor gene is PTEN, Retinoblastoma RB (or RB1), TP53, CDKN2A (INK4A). MLH1, MSH2, MSH6, WT1, WT2, NF1, NF2N, VHL, KLF4, pVHL, APC, CD95, ST5, YPEL3, ST7, APC, MADR2, BRCA1, BRCA2, Patched, TSC1, TSC2, PALB2, or ST14.

In some embodiments, there is provided an mRNA delivery nanoparticle for intracellular delivery of an mRNA comprising a cell-penetrating peptide associated with the mRNA, wherein the cell-penetrating peptide comprises the amino acid sequence of a PEP-1 peptide, a PEP-2 peptide, a VEPEP-3 peptide, a VEPEP-6 peptide, a VEPEP-9 peptide, or an ADGN-100 peptide, and wherein the mRNA encodes a protein, wherein the deficiency of the protein results in a disease or disorder. In some embodiments, the PEP-1 peptide comprises the amino acid sequence of SEQ ID NO: 71. In some embodiments, the PEP-2 peptide comprises the amino acid sequence of SEQ ID NO: 72. In some embodiments, the PEP-3 peptide comprises the amino acid sequence of SEQ ID NO: 73. In some embodiments, the VEPEP-3 peptide comprises the amino acid sequence of any one of SEQ ID NOs: 1-14, 75, and 76. In some embodiments, the VEPEP-6 peptide comprises the amino acid sequence of any one of SEQ ID NOs: 15-40, and 77. In some embodiments, the VEPEP-9 peptide comprises the amino acid sequence of any one of SEQ ID NOs: 41-52, and 78. In some embodiments, the ADGN-100 peptide comprises the amino acid sequence of any one of SEQ ID NOs: 53-70, 79, and 80. In some embodiments, the protein is Frataxin. In some embodiments, the protein is alpha 1 antitrypsin. In some embodiments, the protein is factor VIII. In some embodiments, the protein is factor IX.

In some embodiments, there is provided an mRNA delivery nanoparticle for intracellular delivery of an mRNA comprising a cell-penetrating peptide associated with the mRNA, wherein the cell-penetrating peptide comprises the amino acid sequence of a PEP-1 peptide, a PEP-2 peptide, a VEPEP-3 peptide, a VEPEP-6 peptide, a VEPEP-9 peptide, or an ADGN-100 peptide, and wherein the mRNA encodes a protein, wherein expression of the protein in an individual modulates an immune response to the protein in the individual. In some embodiments, the PEP-1 peptide comprises the amino acid sequence of SEQ ID NO: 71. In some embodiments, the PEP-2 peptide comprises the amino acid sequence of SEQ ID NO: 72. In some embodiments, the PEP-3 peptide comprises the amino acid sequence of SEQ ID NO: 73. In some embodiments, the VEPEP-3 peptide comprises the amino acid sequence of any one of SEQ ID NOs: 1-14, 75, and 76. In some embodiments, the VEPEP-6 peptide comprises the amino acid sequence of any one of SEQ ID NOs: 15-40, and 77. In some embodiments, the VEPEP-9 peptide comprises the amino acid sequence of any one of SEQ ID NOs: 41-52, and 78. In some embodiments, the ADGN-100 peptide comprises the amino acid sequence of any one of SEQ ID NOs: 53-70, 79, and 80. In some embodiments, the protein is an antigen. In some embodiments, the antigen is a disease-associated antigen (e.g., a tumor-associated antigen), and expression of the antigen in the individual results in an increased immune response to the antigen in the individual. In some embodiments, the antigen is a self-antigen, and expression of the antigen in the individual results in a decreased immune response to the antigen in the individual.

In some embodiments, there is provided an mRNA delivery nanoparticle for intracellular delivery of an mRNA comprising a cell-penetrating peptide associated with the mRNA, wherein the cell-penetrating peptide comprises the amino acid sequence of a PEP-1 peptide, a PEP-2 peptide, a VEPEP-3 peptide, a VEPEP-6 peptide, a VEPEP-9 peptide, or an ADGN-100 peptide, and wherein the mRNA encodes an antibody or antigen-binding fragment thereof. In some embodiments, the PEP-1 peptide comprises the amino acid sequence of SEQ ID NO: 71. In some embodiments, the PEP-2 peptide comprises the amino acid sequence of SEQ ID NO: 72. In some embodiments, the PEP-3 peptide comprises the amino acid sequence of SEQ ID NO: 73. In some embodiments, the VEPEP-3 peptide comprises the amino acid sequence of any one of SEQ ID NOs: 1-14, 75, and 76. In some embodiments, the VEPEP-6 peptide comprises the amino acid sequence of any one of SEQ ID NOs: 15-40, and 77. In some embodiments, the VEPEP-9 peptide comprises the amino acid sequence of any one of SEQ ID NOs: 41-52, and 78. In some embodiments, the ADGN-100 peptide comprises the amino acid sequence of any one of SEQ ID NOs: 53-70, 79, and 80. In some embodiments, the antibody is a therapeutic antibody. In some embodiments, the antibody is a bispecific antibody, such as a bispecific T cell engager (BiTE). In some embodiments, the antibody specifically binds to a disease-associated antigen, such as a tumor-associated antigen.

In some embodiments, there is provided an mRNA delivery nanoparticle for intracellular delivery of an mRNA comprising a cell-penetrating peptide associated with the mRNA, wherein the cell-penetrating peptide comprises the amino acid sequence of a PEP-1 peptide, a PEP-2 peptide, a VEPEP-3 peptide, a VEPEP-6 peptide, a VEPEP-9 peptide, or an ADGN-100 peptide, and wherein the mRNA comprises a reporter mRNA. In some embodiments, the PEP-1 peptide comprises the amino acid sequence of SEQ ID NO: 71. In some embodiments, the PEP-2 peptide comprises the amino acid sequence of SEQ ID NO: 72. In some embodiments, the PEP-3 peptide comprises the amino acid sequence of SEQ ID NO: 73. In some embodiments, the VEPEP-3 peptide comprises the amino acid sequence of any one of SEQ ID NOs: 1-14, 75, and 76. In some embodiments, the VEPEP-6 peptide comprises the amino acid sequence of any one of SEQ ID NOs: 15-40, and 77. In some embodiments, the VEPEP-9 peptide comprises the amino acid sequence of any one of SEQ ID NOs: 41-52, and 78. In some embodiments, the ADGN-100 peptide comprises the amino acid sequence of any one of SEQ ID NOs: 53-70, 79, and 80. In some embodiments, the mRNA comprises a EGFP mRNA, for example. CleanCap EGFP mRNA, CleanCap EGFP mRNA (5moU), or CleanCap Cyanine 5 EGFP mRNA (5moU). In some embodiments, the mRNA comprises a Luc mRNA, for example, CleanCap Fluc mRNA, CleanCap Fluc mRNA (5moU), CleanCap Cyanine 5 Fluc mRNA (5moU), CleanCap Gaussia Luc mRNA (5moU), or CleanCap Renilla Luc mRNA (5moU). In some embodiments, the mRNA comprises an mRNA selected from CleanCap β-gal mRNA, CleanCap β-gal mRNA (5moU) and CleanCap mCherry mRNA (5mOU).

In some embodiments, the nanoparticle further comprises an RNAi, such as an RNAi targeting an endogenous gene, e.g., a disease-associated endogenous gene. In some embodiments, the RNAi targets an exogenous gene. In some embodiments, the RNAi comprises an siRNA. In some embodiments, the RNAi comprises a microRNA. In some embodiments, the RNAi targets an oncogene. In some embodiments, the oncogene is Smoothened. In some embodiments, the oncogene is rasK. In some embodiments, the oncogene is KRAS.

In some embodiments, the nanoparticle comprises an mRNA encoding a first protein and an RNAi targeting a second protein. In some embodiments, the RNAi is a therapeutic RNAi targeting an endogenous gene involved in a disease or condition, and the protein is a therapeutic protein useful for treating the disease or condition. In some embodiments, the RNAi targets an exogenous gene. In some embodiments, the therapeutic RNAi targets a disease-associated form of the endogenous gene (e.g., a gene encoding a mutant protein, or a gene resulting in abnormal expression of a protein). In some embodiments, the mRNA corresponds to a therapeutic form of the endogenous gene (e.g., the mRNA encodes a wild-type or functional form of the mutant protein, or the mRNA results in normal expression of the protein). In some embodiments, the one or more cell-penetrating peptides include, but are not limited to, a PTD-based peptide, an amphipathic peptide, a poly-arginine-based peptide, an MPG peptide, a CADY peptide, a VEPEP peptide (such as a VEPEP-3, VEPEP-6, or VEPEP-9 peptide), an ADGN-100 peptide, a Pep-1 peptide, and a Pep-2 peptide.

In some embodiments, there is provided a nanoparticle comprising a core comprising one or more cell-penetrating peptides (e.g., a PEP-1, PEP-2, VEPEP-3, VEPEP-6, VEPEP-9, or ADGN-100 peptide) and a plurality of mRNA, wherein each of the plurality of mRNA encodes a different protein. In some embodiments, the nanoparticle core comprises one of the one or more cell-penetrating peptides associated with at least one of the plurality of mRNA. In some embodiments, the nanoparticle core comprises a) a first complex comprising one of the one or more cell-penetrating peptides associated with at least one of the plurality of mRNA, and b) one or more additional complexes comprising the remaining cell-penetrating peptides associated with the remaining mRNA. In some embodiments, at least some of the one or more cell-penetrating peptides in the nanoparticle are linked to a targeting moiety. In some embodiments, the linkage is covalent. In some embodiments, the covalent linkage is by chemical coupling. In some embodiments, the covalent linkage is by genetic methods. In some embodiments, the molar ratio of a cell-penetrating peptide to an mRNA associated with the cell-penetrating peptide in a complex present in the nanoparticle is between about 1:1 and about 100:1, or between about 1:1 and about 50:1, or about 20:1. In some embodiments, one of the one or more mRNA encodes a therapeutic protein, i.e., a tumor suppressor protein. In some embodiments, the one or more cell-penetrating peptides include, but are not limited to, a PTD-based peptide, an amphipathic peptide, a poly-arginine-based peptide, an MPG peptide, a CADY peptide, a VEPEP peptide (such as a VEPEP-3, VEPEP-6, or VEPEP-9 peptide), an ADGN-100 peptide, a Pep-1 peptide, and a Pep-2 peptide.

In some embodiments, there is provided a nanoparticle for intracellular delivery of an mRNA comprising a core comprising a cell-penetrating peptide and an mRNA, wherein the cell-penetrating peptide is associated with the mRNA, and wherein the cell-penetrating peptide comprises the amino acid sequence of a PEP-1 peptide, a PEP-2 peptide, a VEPEP-3 peptide, a VEPEP-6 peptide, a VEPEP-9 peptide, or an ADGN-100 peptide. In some embodiments, the PEP-1 peptide comprises the amino acid sequence of SEQ ID NO: 71. In some embodiments, the PEP-2 peptide comprises the amino acid sequence of SEQ ID NO: 72. In some embodiments, the PEP-3 peptide comprises the amino acid sequence of SEQ ID NO: 73. In some embodiments, the VEPEP-3 peptide comprises the amino acid sequence of any one of SEQ ID NOs: 1-14, 75, and 76. In some embodiments, the VEPEP-6 peptide comprises the amino acid sequence of any one of SEQ ID NOs: 15-40, and 77. In some embodiments, the VEPEP-9 peptide comprises the amino acid sequence of any one of SEQ ID NOs: 41-52, and 78. In some embodiments, the ADGN-100 peptide comprises the amino acid sequence of any one of SEQ ID NOs: 53-70, 79, and 80.

In some embodiments, the nanoparticle further comprises a surface layer comprising a peripheral CPP surrounding the core. In some embodiments, the peripheral CPP is the same as a CPP in the core. In some embodiments, the peripheral CPP is different than any of the CPPs in the core. In some embodiments, the peripheral CPP includes, but is not limited to, a PTD-based peptide, an amphipathic peptide, a poly-arginine-based peptide, an MPG peptide, a CADY peptide, a VEPEP peptide (such as a VEPEP-3. VEPEP-6, or VEPEP-9 peptide), an ADGN-100 peptide, a Pep-1 peptide, and a Pep-2 peptide. In some embodiments, the peripheral CPP is a VEPEP-3 peptide, a VEPEP-6 peptide, a VEPEP-9 peptide, or an ADGN-100 peptide. In some embodiments, at least some of the peripheral cell-penetrating peptides in the surface layer are linked to a targeting moiety. In some embodiments, the linkage is covalent. In some embodiments, the covalent linkage is by chemical coupling. In some embodiments, the covalent linkage is by genetic methods. In some embodiments, the nanoparticle further comprises an intermediate layer between the core of the nanoparticle and the surface layer. In some embodiments, the intermediate layer comprises an intermediate CPP. In some embodiments, the intermediate CPP is the same as a CPP in the core. In some embodiments, the intermediate CPP is different than any of the CPPs in the core. In some embodiments, the intermediate CPP includes, but is not limited to, a PTD-based peptide, an amphipathic peptide, a poly-arginine-based peptide, an MPG peptide, a CADY peptide, a VEPEP peptide (such as a VEPEP-3, VEPEP-6, or VEPEP-9 peptide), an ADGN-100 peptide, a Pep-1 peptide, and a Pep-2 peptide. In some embodiments, the intermediate CPP is a VEPEP-3 peptide, a VEPEP-6 peptide, a VEPEP-9 peptide, or an ADGN-100 peptide.

In some embodiments, according to any of the nanoparticles described herein, the mean size (diameter) of the nanoparticle is from about 20 nm to about 1000 nm, including for example from about 50 nm to about 800 nm, from about 75 nm to about 600 nm, from about 100 nm to about 600 nm, and from about 200 nm to about 400 nm. In some embodiments, the mean size (diameter) of the nanoparticle is no greater than about 1000 nanometers (nm), such as no greater than about any of 900, 800, 700, 600, 500, 400, 300, 200, or 100 nm. In some embodiments, the average or mean diameter of the nanoparticle is no greater than about 200 nm. In some embodiments, the average or mean diameters of the nanoparticles is no greater than about 150 nm. In some embodiments, the average or mean diameter of the nanoparticle is no greater than about 100 nm. In some embodiments, the average or mean diameter of the nanoparticle is about 20 nm to about 400 nm. In some embodiments, the average or mean diameter of the nanoparticle is about 30 nm to about 400 nm. In some embodiments, the average or mean diameter of the nanoparticle is about 40 nm to about 300 nm. In some embodiments, the average or mean diameter of the nanoparticle is about 50 nm to about 200 nm. In some embodiments, the average or mean diameter of the nanoparticle is about 60 nm to about 150 nm. In some embodiments, the average or mean diameter of the nanoparticle is about 70 nm to about 100 nm. In some embodiments, the nanoparticles are sterile-filterable.

In some embodiments, the zeta potential of the nanoparticle is from about −30 mV to about 60 mV (such as about any of −30, −25, −20, −15, −10, −5, 0, 5, 10, 15, 20, 25, 30, 35, 40, 45, 50, 55, and 60 mV, including any ranges between these values). In some embodiments, the zeta potential of the nanoparticle is from about −30 mV to about 30 mV, including for example from about −25 mV to about 25 mV, from about −20 mV to about 20 mV, from about −15 mV to about 15 mV, from about −10 mV to about 10 mV, and from about −5 mV to about 10 mV. In some embodiments, the polydispersity index (PI) of the nanoparticle is from about 0.05 to about 0.6 (such as about any of 0.05, 0.1, 0.15, 0.2, 0.25, 0.3, 0.35, 0.4, 0.45, 0.5, 0.55, and 0.6, including any ranges between these values). In some embodiments, the nanoparticle is substantially non-toxic.

### Modifications

In some embodiments, an mRNA delivery complex or nanoparticle as described herein comprises a targeting moiety, wherein the targeting moiety is a ligand capable of cell-specific and/or nuclear targeting. A cell membrane surface receptor and/or cell surface marker is a molecule or structure which can bind said ligand with high affinity and preferably with high specificity. Said cell membrane surface receptor and/or cell surface marker is preferably specific for a particular cell, i.e. it is found predominantly in one type of cell rather than in another type of cell (e.g. galactosyl residues to target the asialoglycoprotein receptor on the surface of hepatocytes). The cell membrane surface receptor facilitates cell targeting and intemalization into the target cell of the ligand (e.g. the targeting moiety) and attached molecules (e.g. the complex or nanoparticle of the invention). A large number of ligand moieties/ligand binding partners that may be used in the context of the present invention are widely described in the literature. Such a ligand moiety is capable of conferring to the complex or nanoparticle of the invention the ability to bind to a given binding-partner molecule or a class of binding-partner molecules localized at the surface of at least one target cell. Suitable binding-partner molecules include without limitation polypeptides selected from the group consisting of cell-specific markers, tissue-specific markers, cellular receptors, viral antigens, antigenic epitopes and tumor-associated markers. Binding-partner molecules may moreover consist of or comprise, for example, one or more sugar, lipid, glycolipid, antibody molecules or fragments thereof, or aptamer. According to the invention, a ligand moiety may be for example a lipid, a glycolipid, a hormone, a sugar, a polymer (e.g. PEG, polylysine, PET), an oligonucleotide, a vitamin, an antigen, all or part of a lectin, all or part of a polypeptide, such as for example JTS1 (WO 94/40958), an antibody or a fragment thereof, or a combination thereof. In some embodiments, the ligand moiety used in the present invention is a peptide or polypeptide having a minimal length of 7 amino acids. It is either a native polypeptide or a polypeptide derived from a native polypeptide. “Derived” means containing (a) one or more modifications with respect to the native sequence (e.g. addition, deletion and/or substitution of one or more residues), (b) amino acid analogs, including non-naturally occurring amino acids, (c) substituted linkages, or (d) other modifications known in the art. The polypeptides serving as ligand moiety encompass variant and chimeric polypeptides obtained by fusing sequences of various origins, such as for example a humanized antibody which combines the variable region of a mouse antibody and the constant region of a human immunoglobulin. In addition, such polypeptides may have a linear or cyclized structure (e.g. by flanking at both extremities a polypeptide ligand by cysteine residues). Additionally, the polypeptide in use as a ligand moiety may include modifications of its original structure by way of substitution or addition of chemical moieties (e.g. glycosylation, alkylation, acetylation, amidation, phosphorylation, addition of sulfhydryl groups and the like). The invention further contemplates modifications that render the ligand moiety detectable. For this purpose, modifications with a detectable moiety can be envisaged (i.e. a scintigraphic, radioactive, or fluorescent moiety, or a dye label and the like). Such detectable labels may be attached to the ligand moiety by any conventional techniques and may be used for diagnostic purposes (e.g. imaging of tumoral cells). In some embodiments, the binding-partner molecule is an antigen (e.g. a target cell-specific antigen, a disease-specific antigen, an antigen specifically expressed on the surface of engineered target cells) and the ligand moiety is an antibody, a fragment or a minimal recognition unit thereof (e.g. a fragment still presenting an antigenic specificity) such as those described in detail in immunology manuals (see for example Immunology, third edition 1993, Roitt, Brostoff and Male, ed Gambli, Mosby). The ligand moiety may be a monoclonal antibody. Many monoclonal antibodies that bind many of these antigens are already known, and using techniques known in the art in relation to monoclonal antibody technology, antibodies to most antigens may be prepared. The ligand moiety may be a part of an antibody (for example a Fab fragment) or a synthetic antibody fragment (for example, ScFv). In some embodiments, the ligand moiety is selected among antibody fragments, rather than whole antibodies. Effective functions of whole antibodies, such as complement binding, are removed. ScFv and dAb antibody fragments may be expressed as a fusion with one or more other polypeptides. Minimal recognition units may be derived from the sequence of one or more of the complementary-determining regions (CDR) of the Fv fragment. Whole antibodies, and F(ab′)2 fragments are “bivalent”. By “bivalent” it is meant that said antibodies and F(ab′)2 fragments have two antigen binding sites. In contrast, Fab, Fv, ScFv, dAb fragments and minimal recognition units are monovalent, having only one antigen binding sites. In some embodiments, the ligand moiety allows targeting to a tumor cell and is capable of recognizing and binding to a molecule related to the tumor status, such as a tumor-specific antigen, a cellular protein differentially or over-expressed in tumor cells or a gene product of a cancer-associated vims. Examples of tumor-specific antigens include but are not limited to MUC-1 related to breast cancer (Hareuven i et al., 990, Eur. J. Biochem 189, 475-486), the products encoded by the mutated BRCA1 and BRCA2 genes related to breast and ovarian cancers (Miki et al, 1994, Science 226, 66-7 1; Fuireal et al, 1994, Science 226, 120-122: Wooster et al., 1995, Nature 378, 789-792), APC related to cancer (Poiakis, 1995, Curr. Opin. Genet. Dev. 5, 66-71), prostate specific antigen (PSA) related to prostate cancer, (Stamey et al., 1987. New England J. Med. 317, 909), carcinoma embryonic antigen (CEA) related to cancers (Schrewe et aL., 1990, Mol. Cell. Biol. 10, 2738-2748), tyrosinase related to melanomas (Vile et al, 1993, Cancer Res. 53, 3860-3864), receptor for melanocyte-stimulating hormone (MSH) which is highly expressed in melanoma cells, ErbB-2 related to breast and pancreas cancers (Harris et al., 1994, Gene Therapy 1, 170-175), and alpha-foetoprotein related to liver cancers (Kanai et al., 1997, Cancer Res. 57, 46 1-465). In some embodiments, the ligand moiety is a fragment of an antibody capable of recognizing and binding to the MUC-1 antigen and thus targeting MUC-1 positive tumor cells. In some embodiments, the ligand moiety is the scFv fragment of the SM3 monoclonal antibody which recognizes the tandem repeat region of the MUC-1 antigen (Burshell et aL., 1987, Cancer Res. 47, 5476-5482; Girling et aL., 1989, Int. J. Cancer 43, 1072-1076; Dokurno et aL., 1998, J. Mol. Biol. 284, 713-728). Examples of cellular proteins differentially or overexpressed in tumor cells include but are not limited to the receptor for interleukin 2 (IL-2) overexpressed in some lymphoid tumors, GRP (Gastrin Release Peptide) overexpressed in lung carcinoma cells, pancreas, prostate and stomach tumors (Michael et al., 1995, Gene Therapy 2, 660-668), TNF (Tumor Necrosis Factor) receptor, epidermal growth factor receptors, Fas receptor, CD40 receptor, CD30 receptor, CD27 receptor, OX-40, α-v integrins (Brooks et al, 994, Science 264, 569) and receptors for certain angiogenic growth factors (Hanahan, 1997, Science 277, 48). Based on these indications, it is within the scope of those skilled in the art to define an appropriate ligand moiety capable of recognizing and binding to such proteins. To illustrate, IL-2 is a suitable ligand moiety to bind to TL-2 receptor. In the case of receptors that are specific to fibrosis and inflammation, these include the TGFbeta receptors or the Adenosine receptors that are identified above and are suitable targets for invention compositions. Cell surface markers for multiple myeloma include, but are not limited to, CD56, CD40, FGFR3, CS1, CD138, IGF1R VEGFR, and CD38, and are suitable targets for invention compositions. Suitable ligand moieties that bind to these cell surface markers include, but are not limited to, anti-CD56, anti-CD40, PRO-001, Chir-258, HuLuc63, anti-CD138-DML, anti-IGF1R and bevacizumab.

mRNA or RNAi Compositions

In some embodiments, there is provided a composition (e.g., a pharmaceutical composition) comprising an mRNA or RNAi (e.g., siRNA) delivery complex or nanoparticle as described herein. In some embodiments, the composition is a pharmaceutical composition comprising an mRNA or RNAi (e.g., siRNA) delivery complex or nanoparticle as described herein and a pharmaceutically acceptable diluent, excipient, and/or carrier. In some embodiments, the concentration of the complex or nanoparticle in the composition is from about 1 nM to about 100 mM, including for example from about 10 nM to about 50 mM, from about 25 nM to about 25 mM, from about 50 nM to about 10 mM, from about 100 nM to about 1 mM, from about 500 nM to about 750 μM, from about 750 nM to about 500 μM, from about 1 μM to about 250 μM, from about 10 μM to about 200 μM, and from about 50 μM to about 150 μM. In some embodiments, the pharmaceutical composition is lyophilized.

The term “pharmaceutically acceptable diluent, excipient, and/or carrier” as used herein is intended to include any and all solvents, dispersion media, coatings, antibacterial and antifungal agents, isotonic and absorption delaying agents, and the like, compatible with administration to humans or other vertebrate hosts. Typically, a pharmaceutically acceptable diluent, excipient, and/or carrier is a diluent, excipient, and/or carrier approved by a regulatory agency of a Federal, a state government, or other regulatory agency, or listed in the U.S. Pharmacopeia or other generally recognized pharmacopeia for use in animals, including humans as well as non-human mammals. The term diluent, excipient, and/or “carrier” refers to a diluent, adjuvant, excipient, or vehicle with which the pharmaceutical composition is administered. Such pharmaceutical diluent, excipient, and/or carriers can be sterile liquids, such as water and oils, including those of petroleum, animal, vegetable or synthetic origin. Water, saline solutions and aqueous dextrose and glycerol solutions can be employed as liquid diluents, excipients, and/or carriers, particularly for injectable solutions. Suitable pharmaceutical diluents and/or excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like, including lyophilization aids. The composition, if desired, can also contain minor amounts of wetting, bulking, emulsifying agents, or pH buffering agents. These compositions can take the form of solutions, suspensions, emulsion, sustained release formulations and the like. Examples of suitable pharmaceutical diluent, excipient, and/or carriers are described in “Remington's Pharmaceutical Sciences” by E. W. Martin. The formulation should suit the mode of administration. The appropriate diluent, excipient, and/or carrier will be evident to those skilled in the art and will depend in large part upon the route of administration.

In some embodiments, a composition comprising an mRNA or RNAi (e.g., siRNA) delivery complex or nanoparticle as described herein further comprises a pharmaceutically acceptable diluent, excipient, and/or carrier. In some embodiments, the pharmaceutically acceptable diluent, excipient, and/or carrier affects the level of aggregation of an mRNA delivery complex or nanoparticle in the composition and/or the efficiency of intracellular delivery mediated by an mRNA or RNAi (e.g., siRNA) delivery complex or nanoparticle in the composition. In some embodiments, the extent and/or direction of the effect on aggregation and/or delivery efficiency mediated by the pharmaceutically acceptable diluent, excipient, and/or carrier is dependent on the relative amount of the pharmaceutically acceptable diluent, excipient, and/or carrier in the composition.

For example, in some embodiments, the presence of a pharmaceutically acceptable diluent, excipient, and/or carrier (such as a salt, sugar, chemical buffering agent, buffer solution, cell culture medium, or carrier protein) at one or more concentrations in the composition does not promote and/or contribute to aggregation of the mRNA or RNAi (e.g., siRNA) delivery complex or nanoparticle, or promotes and/or contributes to the formation of aggregates of the mRNA or RNAi (e.g., siRNA) delivery complex or nanoparticles having a size no more than about 200% (such as no more than about any of 190, 180, 170, 160, 150, 140, 130, 120, 110, 100, 90, 80, 70, 60, 50, 40, 30, 20, 10, 9, 8, 7, 6, 5, 4, 3, 2, or 1%, including any ranges between any of these values) larger than the size of the mRNA or RNAi (e.g., siRNA) delivery complex or nanoparticle. In some embodiments, the composition comprises the pharmaceutically acceptable diluent, excipient, and/or carrier at a concentration that does not promote and/or contribute to aggregation of the mRNA or RNAi (e.g., siRNA) delivery complex or nanoparticle, or promotes and/or contributes to the formation of aggregates of the mRNA or RNAi (e.g., siRNA) delivery complex or nanoparticles having a size no more than about 200% (such as no more than about any of 190, 180, 170, 160, 150, 140, 130, 120, 110, 100, 90, 80, 70, 60, 50, 40, 30, 20, 10, 9, 8, 7, 6, 5, 4, 3, 2, or 1%, including any ranges between any of these values) larger than the size of the mRNA or RNAi (e.g., siRNA) delivery complex or nanoparticle. In some embodiments, the composition comprises the pharmaceutically acceptable diluent, excipient, and/or carrier at a concentration that promotes and/or contributes to the formation of aggregates of the mRNA or RNAi (e.g., siRNA) delivery complex or nanoparticles having a size no more than about 150% larger than the size of the mRNA or RNAi (e.g., siRNA) delivery complex or nanoparticle. In some embodiments, the composition comprises the pharmaceutically acceptable diluent, excipient, and/or carrier at a concentration that promotes and/or contributes to the formation of aggregates of the mRNA or RNAi (e.g., siRNA) delivery complex or nanoparticles having a size no more than about 100% larger than the size of the mRNA or RNAi (e.g., siRNA) delivery complex or nanoparticle. In some embodiments, the composition comprises the pharmaceutically acceptable diluent, excipient, and/or carrier at a concentration that promotes and/or contributes to the formation of aggregates of the mRNA or RNAi (e.g., siRNA) delivery complex or nanoparticles having a size no more than about 50% larger than the size of the mRNA or RNAi (e.g., siRNA) delivery complex or nanoparticle. In some embodiments, the composition comprises the pharmaceutically acceptable diluent, excipient, and/or carrier at a concentration that promotes and/or contributes to the formation of aggregates of the mRNA or RNAi (e.g., siRNA) delivery complex or nanoparticles having a size no more than about 20% larger than the size of the mRNA or RNAi (e.g., siRNA) delivery complex or nanoparticle. In some embodiments, the composition comprises the pharmaceutically acceptable diluent, excipient, and/or carrier at a concentration that promotes and/or contributes to the formation of aggregates of the mRNA or RNAi (e.g., siRNA) delivery complex or nanoparticles having a size no more than about 15% larger than the size of the mRNA or RNAi (e.g., siRNA) delivery complex or nanoparticle. In some embodiments, the composition comprises the pharmaceutically acceptable diluent, excipient, and/or carrier at a concentration that promotes and/or contributes to the formation of aggregates of the mRNA or RNAi (e.g., siRNA) delivery complex or nanoparticles having a size no more than about 10% larger than the size of the mRNA or RNAi (e.g., siRNA) delivery complex or nanoparticle. In some embodiments, the pharmaceutically acceptable diluent, excipient, and/or carrier is a salt, including, without limitation, NaCl. In some embodiments, the pharmaceutically acceptable diluent, excipient, and/or carrier is a sugar, including, without limitation, sucrose, glucose, and mannitol. In some embodiments, the pharmaceutically acceptable diluent, excipient, and/or carrier is a chemical buffering agent, including, without limitation. HEPES. In some embodiments, the pharmaceutically acceptable diluent, excipient, and/or carrier is a buffer solution, including, without limitation, PBS. In some embodiments, the pharmaceutically acceptable diluent, excipient, and/or carrier is a cell culture medium, including, without limitation, DMEM. Particle size can be determined using any means known in the art for measuring particle size, such as by dynamic light scattering (DLS). For example, in some embodiments, an aggregate having a Z-average as measured by DLS that is 10% greater than the Z-average as measured by DLS of an mRNA or RNAi (e.g., siRNA) delivery complex or nanoparticle is 10% larger than the mRNA delivery complex or nanoparticle.

In some embodiments, the composition comprises a salt (e.g., NaCl) at a concentration that does not promote and/or contribute to aggregation of the mRNA or RNAi (e.g., siRNA) delivery complex or nanoparticle, or promotes and/or contributes to the formation of aggregates of the mRNA or RNAi (e.g., siRNA) delivery complex or nanoparticles having a size no more than about 100% (such as no more than about any of 90, 80, 70, 60, 50, 40, 30, 20, 10, 9, 8, 7, 6, 5, 4, 3, 2, or 1%, including any ranges between any of these values) larger than the size of the mRNA or RNAi (e.g., siRNA) delivery complex or nanoparticle. In some embodiments, the composition comprises a salt (e.g., NaCl) at a concentration that promotes and/or contributes to the formation of aggregates of the mRNA or RNAi (e.g., siRNA) delivery complex or nanoparticles having a size no more than about 75% larger than the size of the mRNA or RNAi (e.g., siRNA) delivery complex or nanoparticle. In some embodiments, the composition comprises a salt (e.g., NaCl) at a concentration that promotes and/or contributes to the formation of aggregates of the mRNA or RNAi (e.g., siRNA) delivery complex or nanoparticles having a size no more than about 50% larger than the size of the mRNA or RNAi (e.g., siRNA) delivery complex or nanoparticle. In some embodiments, the composition comprises a salt (e.g., NaCl) at a concentration that promotes and/or contributes to the formation of aggregates of the mRNA or RNAi (e.g., siRNA) delivery complex or nanoparticles having a size no more than about 20% larger than the size of the mRNA or RNAi (e.g., siRNA) delivery complex or nanoparticle. In some embodiments, the composition comprises a salt (e.g., NaCl) at a concentration that promotes and/or contributes to the formation of aggregates of the mRNA or RNAi (e.g., siRNA) delivery complex or nanoparticles having a size no more than about 15% larger than the size of the mRNA or RNAi (e.g., siRNA) delivery complex or nanoparticle. In some embodiments, the composition comprises a salt (e.g., NaCl) at a concentration that promotes and/or contributes to the formation of aggregates of the mRNA or RNAi (e.g., siRNA) delivery complex or nanoparticles having a size no more than about 10% larger than the size of the mRNA or RNAi (e.g., siRNA) delivery complex or nanoparticle. In some embodiments, the concentration of the salt in the composition is no more than about 100 mM (such as no more than about any of 90, 80, 70, 60, 50, 40, 30, 20, 10, 9, 8, 7, 6, 5, 4, 3, 2, or mM, including any ranges between any of these values). In some embodiments, the salt is NaCl.

In some embodiments, the composition comprises a sugar (e.g., sucrose, glucose, or mannitol) at a concentration that does not promote and/or contribute to aggregation of the mRNA or RNAi (e.g., siRNA) delivery complex or nanoparticle, or promotes and/or contributes to the formation of aggregates of the mRNA or RNAi (e.g., siRNA) delivery complex or nanoparticles having a size no more than about 25% (such as no more than about any of 24, 23, 22, 21, 20, 19, 18, 17, 16, 15, 14, 13, 12, 11, 10, 9, 8, 7, 6, 5, 4, 3, 2, or 1%, including any ranges between any of these values) larger than the size of the mRNA or RNAi (e.g., siRNA) delivery complex or nanoparticle. In some embodiments, the composition comprises a sugar (e.g., sucrose, glucose, or mannitol) at a concentration that promotes and/or contributes to the formation of aggregates of the mRNA or RNAi (e.g., siRNA) delivery complex or nanoparticles having a size no more than about 75% larger than the size of the mRNA or RNAi (e.g., siRNA) delivery complex or nanoparticle. In some embodiments, the composition comprises a sugar (e.g., sucrose, glucose, or mannitol) at a concentration that promotes and/or contributes to the formation of aggregates of the mRNA or RNAi (e.g., siRNA) delivery complex or nanoparticles having a size no more than about 50% larger than the size of the mRNA or RNAi (e.g., siRNA) delivery complex or nanoparticle. In some embodiments, the composition comprises a sugar (e.g., sucrose, glucose, or mannitol) at a concentration that promotes and/or contributes to the formation of aggregates of the mRNA or RNAi (e.g., siRNA) delivery complex or nanoparticles having a size no more than about 20% larger than the size of the mRNA or RNAi (e.g., siRNA) delivery complex or nanoparticle. In some embodiments, the composition comprises a sugar (e.g., sucrose, glucose, or mannitol) at a concentration that promotes and/or contributes to the formation of aggregates of the mRNA or RNAi (e.g., siRNA) delivery complex or nanoparticles having a size no more than about 15% larger than the size of the mRNA or RNAi (e.g., siRNA) delivery complex or nanoparticle. In some embodiments, the composition comprises a sugar (e.g., sucrose, glucose, or mannitol) at a concentration that promotes and/or contributes to the formation of aggregates of the mRNA or RNAi (e.g., siRNA) delivery complex or nanoparticles having a size no more than about 10% larger than the size of the mRNA or RNAi (e.g., siRNA) delivery complex or nanoparticle. In some embodiments, the concentration of the sugar in the composition is no more than about 20% (such as no more than about any of 18, 16, 14, 12, 10, 9, 8, 7, 6, 5, 4, 3, 2, or 1%, including any ranges between any of these values). In some embodiments, the sugar is sucrose. In some embodiments, the sugar is glucose. In some embodiments, the sugar is mannitol.

In some embodiments, the composition comprises a chemical buffering agent (e.g., HEPES or phosphate) at a concentration that does not promote and/or contribute to aggregation of the mRNA or RNAi (e.g., siRNA) delivery complex or nanoparticle, or promotes and/or contributes to the formation of aggregates of the mRNA or RNAi (e.g., siRNA) delivery complex or nanoparticles having a size no more than about 100% (such as no more than about any of 9, 8, 7, 6, 5, 4, 3, 2, or 1%, including any ranges between any of these values) larger than the size of the mRNA or RNAi (e.g., siRNA) delivery complex or nanoparticle. In some embodiments, the composition comprises a chemical buffering agent (e.g., HEPES or phosphate) at a concentration that promotes and/or contributes to the formation of aggregates of the mRNA or RNAi (e.g., siRNA) delivery complex or nanoparticles having a size no more than about 7.5% larger than the size of the mRNA or RNAi (e.g., siRNA) delivery complex or nanoparticle. In some embodiments, the composition comprises a chemical buffering agent (e.g., HEPES or phosphate) at a concentration that promotes and/or contributes to the formation of aggregates of the mRNA or RNAi (e.g., siRNA) delivery complex or nanoparticles having a size no more than about 5% larger than the size of the mRNA or RNAi (e.g., siRNA) delivery complex or nanoparticle. In some embodiments, the composition comprises a chemical buffering agent (e.g., HEPES or phosphate) at a concentration that promotes and/or contributes to the formation of aggregates of the mRNA or RNAi (e.g., siRNA) delivery complex or nanoparticles having a size no more than about 3% larger than the size of the mRNA or RNAi (e.g., siRNA) delivery complex or nanoparticle. In some embodiments, the composition comprises a chemical buffering agent (e.g., HEPES or phosphate) at a concentration that promotes and/or contributes to the formation of aggregates of the mRNA or RNAi (e.g., siRNA) delivery complex or nanoparticles having a size no more than about 1% larger than the size of the mRNA or RNAi (e.g., siRNA) delivery complex or nanoparticle. In some embodiments, the composition comprises a chemical buffering agent (e.g., HEPES or phosphate) at a concentration that does not promote and/or contribute to the formation of aggregates of the mRNA or RNAi (e.g., siRNA) delivery complex or nanoparticles. In some embodiments, the chemical buffering agent is HEPES. In some embodiments, the HEPES is added to the composition in the form of a buffer solution comprising HEPES. In some embodiments, the solution comprising HEPES has a pH between about 5 and about 9 (such as about any of 5, 5.5, 6, 6.5, 7, 7.5, 8, 8.5, and 9, including any ranges between these values). In some embodiments, the composition comprises HEPES at a concentration of no more than about 75 mM (such as no more than about any of 70, 65, 60, 55, 50, 45, 40, 35, 30, 25, 20, 15, 10 mM or less, including any ranges between any of these values). In some embodiments, the chemical buffering agent is phosphate. In some embodiments, the phosphate is added to the composition in the form of a buffer solution comprising phosphate. In some embodiments, the composition does not comprise PBS.

In some embodiments, the composition comprises a cell culture medium (e.g., DMEM or Opti-MEM) at a concentration that does not promote and/or contribute to aggregation of the mRNA or RNAi (e.g., siRNA) delivery complex or nanoparticle, or promotes and/or contributes to the formation of aggregates of the mRNA or RNAi (e.g., siRNA) delivery complex or nanoparticles having a size no more than about 200% (such as no more than about any of 190, 180, 170, 160, 150, 140, 130, 120, 110, 100, 90, 80, 70, 60, 50, 40, 30, 20, 10, 9, 8, 7, 6, 5, 4, 3, 2, or 1%, including any ranges between any of these values) larger than the size of the mRNA or RNAi (e.g., siRNA) delivery complex or nanoparticle. In some embodiments, the composition comprises a cell culture medium (e.g., DMEM or Opti-MEM) at a concentration that promotes and/or contributes to the formation of aggregates of the mRNA or RNAi (e.g., siRNA) delivery complex or nanoparticles having a size no more than about 150% larger than the size of the mRNA or RNAi (e.g., siRNA) delivery complex or nanoparticle. In some embodiments, the composition comprises a cell culture medium (e.g., DMEM or Opti-MEM) at a concentration that promotes and/or contributes to the formation of aggregates of the mRNA or RNAi (e.g., siRNA) delivery complex or nanoparticles having a size no more than about 100% larger than the size of the mRNA or RNAi (e.g., siRNA) delivery complex or nanoparticle. In some embodiments, the composition comprises a cell culture medium (e.g., DMEM or Opti-MEM) at a concentration that promotes and/or contributes to the formation of aggregates of the mRNA or RNAi (e.g., siRNA) delivery complex or nanoparticles having a size no more than about 50% larger than the size of the mRNA or RNAi (e.g., siRNA) delivery complex or nanoparticle. In some embodiments, the composition comprises a cell culture medium (e.g., DMEM or Opti-MEM) at a concentration that promotes and/or contributes to the formation of aggregates of the mRNA or RNAi (e.g., siRNA) delivery complex or nanoparticles having a size no more than about 25% larger than the size of the mRNA or RNAi (e.g., siRNA) delivery complex or nanoparticle. In some embodiments, the composition comprises a cell culture medium (e.g., DMEM or Opti-MEM) at a concentration that promotes and/or contributes to the formation of aggregates of the mRNA or RNAi (e.g., siRNA) delivery complex or nanoparticles having a size no more than about 10% larger than the size of the mRNA or RNAi (e.g., siRNA) delivery complex or nanoparticle. In some embodiments, the cell culture medium is DMEM. In some embodiments, the composition comprises DMEM at a concentration of no more than about 70% (such as no more than about any of 65, 60, 55, 50, 45, 40, 35, 30, 25, 20, 15, 10%, or less, including any ranges between any of these values).

In some embodiments, the composition comprises a carrier protein (e.g., albumin) at a concentration that does not promote and/or contribute to aggregation of the mRNA or RNAi (e.g., siRNA) delivery complex or nanoparticle, or promotes and/or contributes to the formation of aggregates of the mRNA or RNAi (e.g., siRNA) delivery complex or nanoparticles having a size no more than about 200% (such as no more than about any of 190, 180, 170, 160, 150, 140, 130, 120, 110, 100, 90, 80, 70, 60, 50, 40, 30, 20, 10, 9, 8, 7, 6, 5, 4, 3, 2, or 1%, including any ranges between any of these values) larger than the size of the mRNA or RNAi (e.g., siRNA) delivery complex or nanoparticle. In some embodiments, the composition comprises a carrier protein (e.g., albumin) at a concentration that promotes and/or contributes to the formation of aggregates of the mRNA or RNAi (e.g., siRNA) delivery complex or nanoparticles having a size no more than about 150% larger than the size of the mRNA or RNAi (e.g., siRNA) delivery complex or nanoparticle. In some embodiments, the composition comprises a carrier protein (e.g., albumin) at a concentration that promotes and/or contributes to the formation of aggregates of the mRNA or RNAi (e.g., siRNA) delivery complex or nanoparticles having a size no more than about 100% larger than the size of the mRNA or RNAi (e.g., siRNA) delivery complex or nanoparticle. In some embodiments, the composition comprises a carrier protein (e.g., albumin) at a concentration that promotes and/or contributes to the formation of aggregates of the mRNA or RNAi (e.g., siRNA) delivery complex or nanoparticles having a size no more than about 50% larger than the size of the mRNA or RNAi (e.g., siRNA) delivery complex or nanoparticle. In some embodiments, the composition comprises a carrier protein (e.g., albumin) at a concentration that promotes and/or contributes to the formation of aggregates of the mRNA or RNAi (e.g., siRNA) delivery complex or nanoparticles having a size no more than about 25% larger than the size of the mRNA or RNAi (e.g., siRNA) delivery complex or nanoparticle. In some embodiments, the composition comprises a carrier protein (e.g., albumin) at a concentration that promotes and/or contributes to the formation of aggregates of the mRNA or RNAi (e.g., siRNA) delivery complex or nanoparticles having a size no more than about 10% larger than the size of the mRNA or RNAi (e.g., siRNA) delivery complex or nanoparticle. In some embodiments, the carrier protein is albumin. In some embodiments, the albumin is human serum albumin.

In some embodiments, a pharmaceutical composition as described herein is formulated for intravenous, intratumoral, intraarterial, topical, intraocular, ophthalmic, intraportal, intracranial, intracerebral, intracerebroventricular, intrathecal, intravesicular, intradermal, subcutaneous, intramuscular, intranasal, intratracheal, pulmonary, intracavity, or oral administration.

In some embodiments, dosages of the pharmaceutical compositions of the present invention found to be suitable for treatment of human or mammalian subjects are in the range of about 0.001 mg/kg to about 100 mg/kg (such as about any of 0.001, 0.01, 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 95, and 100 mg/kg, including any ranges between these values) of the mRNA or RNAi (e.g., siRNA) delivery complexes or nanoparticles. In some embodiments, dosage ranges are about 0.1 mg/kg to about 20 mg/kg (such as about any of 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, and 20 mg/kg, including any ranges between these values). In some embodiments, dosage ranges are about 0.5 mg/kg to about 10 mg/kg.

In some embodiments, dosages of the pharmaceutical compositions of the present invention found to be suitable for treatment of human or mammalian subjects are in the range of about 0.03 mg/m² to about 4×10³ mg/m² (such as about any of 0.03, 0.3, 3, 30, 300, 3×10³, and 4×10³ mg/m², including any ranges between these values) of the mRNA or RNAi (e.g., siRNA) delivery complexes or nanoparticles. In some embodiments, dosage ranges are about 3 mg/m² to about 800 mg/m² (such as about any of 3, 30, 300, 600, 800 mg/m², including any ranges between these values). In some embodiments, dosage ranges are about 18 mg/m² to about 400 mg/m².

Exemplary dosing frequencies include, but are not limited to, weekly without break; weekly, three out of four weeks: once every three weeks; once every two weeks: weekly, two out of three weeks. In some embodiments, the pharmaceutical composition is administered about once every 2 weeks, once every 3 weeks, once every 4 weeks, once every 6 weeks, or once every 8 weeks. In some embodiments, the pharmaceutical composition is administered at least about any of 1×, 2×, 3×, 4×, 5×, 6×, or 7× (i.e., daily) a week. In some embodiments, the intervals between each administration are less than about any of 6 months, 3 months, 1 month, 20 days, 15, days, 12 days, 10 days, 9 days, 8 days, 7 days, 6 days, 5 days, 4 days, 3 days, 2 days, or 1 day. In some embodiments, the intervals between each administration are more than about any of 1 month, 2 months, 3 months, 4 months, 5 months, 6 months, 8 months, or 12 months. In some embodiments, there is no break in the dosing schedule. In some embodiments, the interval between each administration is no more than about a week. In some embodiments, the schedule of administration of the pharmaceutical composition to an individual ranges from a single administration that constitutes the entire treatment to daily administration. The administration of the pharmaceutical composition can be extended over an extended period of time, such as from about a month up to about seven years. In some embodiments, the pharmaceutical composition is administered over a period of at least about any of 2, 3, 4, 5, 6, 7, 8, 9, 10, 11, 12, 18, 24, 30, 36, 48, 60, 72, or 84 months.

### Nanoparticle Composition Used as a Second Agent

The nanoparticle compositions used as a second agent described herein comprise nanoparticles comprising (in various embodiments consisting essentially of) a taxane (such as paclitaxel) or an mTOR inhibitor (e.g., rapamycin) and an albumin (such as human serum albumin). Nanoparticles of poorly water soluble drugs (such as taxane) have been disclosed in, for example, U.S. Pat. Nos. 5,916,596; 6,506,405; 6,749,868, and 6,537,579; 7,820,788, and US Pat. Pub. Nos., 2006/0263434, and 2007/0082838; PCT Patent Application WO08/137148, each of which is incorporated by reference in their entirety.

In some embodiments, the composition comprises nanoparticles with an average or mean diameter of no greater than about 1000 nanometers (nm), such as no greater than about any of 900, 800, 700, 600, 500, 400, 300, 200, and 100 nm. In some embodiments, the average or mean diameters of the nanoparticles is no greater than about 200 nm. In some embodiments, the average or mean diameters of the nanoparticles is no greater than about 150 nm. In some embodiments, the average or mean diameters of the nanoparticles is no greater than about 100 nm. In some embodiments, the average or mean diameter of the nanoparticles is about 20 to about 400 nm. In some embodiments, the average or mean diameter of the nanoparticles is about 40 to about 200 nm. In some embodiments, the nanoparticles are sterile-filterable.

In some embodiments, the nanoparticles in the composition described herein have an average diameter of no greater than about 200 nm, including for example no greater than about any one of 190, 180, 170, 160, 150, 140, 130, 120, 110, 100, 90, 80, 70, or 60 nm. In some embodiments, at least about 50% (for example at least about any one of 60%, 70%, 80%, 90%, 95%, or 99%) of the nanoparticles in the composition have a diameter of no greater than about 200 nm, including for example no greater than about any one of 190, 180, 170, 160, 150, 140, 130, 120, 110, 100, 90, 80, 70, or 60 nm. In some embodiments, at least about 50% (for example at least any one of 60%, 70%, 80%, 90%, 95%, or 99%) of the nanoparticles in the composition fall within the range of about 20 to about 400 nm, including for example about 20 to about 200 nm, about 40 to about 200 nm, about 30 to about 180 nm, and any one of about 40 to about 150, about 50 to about 120, and about 60 to about 100 nm.

In some embodiments, the albumin has sulfhydryl groups that can form disulfide bonds. In some embodiments, at least about 5% (including for example at least about any one of 10%, 15%, 20%, 25%, 30%, 40%, 50%, 60%, 70%, 80%, or 90%) of the albumin in the nanoparticle portion of the composition are crosslinked (for example crosslinked through one or more disulfide bonds).

In some embodiments, the nanoparticles comprise the taxane (such as paclitaxel) coated with an albumin (e.g., human serum albumin). In some embodiments, the composition comprises taxane in both nanoparticle and non-nanoparticle forms, wherein at least about any one of 50%, 60%, 70%, 80%, 90%, 95%, or 99% of the taxane in the composition are in nanoparticle form. In some embodiments, the taxane in the nanoparticles constitutes more than about any one of 50%, 60%, 70%, 80%, 90%, 95%, or 99% of the nanoparticles by weight. In some embodiments, the nanoparticles have a non-polymeric matrix. In some embodiments, the nanoparticles comprise a core of taxane that is substantially free of polymeric materials (such as polymeric matrix).

In some embodiments, the composition comprises albumin in both nanoparticle and non-nanoparticle portions of the composition, wherein at least about any one of 50%, 60%, 70%, 80%, 90%, 95%, or 99% of the albumin in the composition are in non-nanoparticle portion of the composition.

In some embodiments, the weight ratio of albumin (such as human serum albumin) and taxane in the nanoparticle composition is about 18:1 or less, such as about 15:1 or less, for example about 10:1 or less. In some embodiments, the weight ratio of albumin (such as human serum albumin) and taxane in the composition falls within the range of any one of about 1:1 to about 18:1, about 2:1 to about 15:1, about 3:1 to about 13:1, about 4:1 to about 12:1, about 5:1 to about 10:1. In some embodiments, the weight ratio of albumin and taxane in the nanoparticle portion of the composition is about any one of 1:2, 1:3, 1:4, 1:5, 1:10, 1:15, or less. In some embodiments, the weight ratio of the albumin (such as human serum albumin) and the taxane in the composition is any one of the following: about 1:1 to about 18:1, about 1:1 to about 15:1, about 1:1 to about 12:1, about 1:1 to about 10:1, about 1:1 to about 9:1, about 1:1 to about 8:1, about 1:1 to about 7:1, about 1:1 to about 6:1, about 1:1 to about 5:1, about 1:1 to about 4:1, about 1:1 to about 3:1, about 1:1 to about 2:1, about 1:1 to about 1:1.

In some embodiments, the nanoparticle composition comprises one or more of the above characteristics.

The nanoparticles described herein may be present in a dry formulation (such as lyophilized composition) or suspended in a biocompatible medium. Suitable biocompatible media include, but are not limited to, water, buffered aqueous media, saline, buffered saline, optionally buffered solutions of amino acids, optionally buffered solutions of proteins, optionally buffered solutions of sugars, optionally buffered solutions of vitamins, optionally buffered solutions of synthetic polymers, lipid-containing emulsions, and the like.

In some embodiments, the pharmaceutically acceptable carrier comprises human serum albumin. Human serum albumin (HSA) is a highly soluble globular protein of Mr 65K and consists of 585 amino acids. HSA is the most abundant protein in the plasma and accounts for 70-80% of the colloid osmotic pressure of human plasma. The amino acid sequence of HSA contains a total of 17 disulphide bridges, one free thiol (Cys 34), and a single tryptophan (Trp 214). Intravenous use of HSA solution has been indicated for the prevention and treatment of hypovolumic shock (see, e.g., Tullis, JAMA, 237, 355-360, 460-463, (1977)) and Houser et al., Surgery, Gynecology and Obstetrics, 50, 811-816 (1980)) and in conjunction with exchange transfusion in the treatment of neonatal hyperbilirubinemia (see, e.g., Finlayson, Seminars in Thrombosis and Hemostasis, 6, 85-120, (1980)). Other albumins are contemplated, such as bovine serum albumin. Use of such non-human albumins could be appropriate, for example, in the context of use of these compositions in non-human mammals, such as the veterinary (including domestic pets and agricultural context).

Human serum albumin (HSA) has multiple hydrophobic binding sites (a total of eight for fatty acids, an endogenous ligand of HSA) and binds a diverse set of taxanes, especially neutral and negatively charged hydrophobic compounds (Goodman et al., The Pharmacological Basis of Therapeutics, 9^{th} ed, McGraw-Hill New York (1996)). Two high affinity binding sites have been proposed in subdomains IIA and IIIA of HSA, which are highly elongated hydrophobic pockets with charged lysine and arginine residues near the surface which function as attachment points for polar ligand features (see, e.g., Fehske et al., Biochem. Pharmcol., 30, 687-92 (198a), Vorum, Dan. Med. Bull., 46, 379-99 (1999), Kragh-Hansen, Dan. Med. Bull., 1441, 131-40 (1990), Curry et al., Nat. Struct. Biol., 5, 827-35 (1998), Sugio et al., Protein. Eng., 12, 439-46 (1999), He et al., Nature, 358, 209-15 (199b), and Carter et al., Adv. Protein. Chem., 45, 153-203 (1994)). Paclitaxel and propofol have been shown to bind HSA (see, e.g., Paal et al., Eur. J. Biochem., 268(7), 2187-91 (200a), Purcell et al., Biochim. Biophys. Acta, 1478(a), 61-8 (2000), Altmayer et al., Arzneimittelforschung, 45, 1053-6 (1995), and Garrido et al., Rev. Esp. Anestestiol. Reanim., 41, 308-12 (1994)). In addition, docetaxel has been shown to bind to human plasma proteins (see, e.g., Urien et al., Invest. New Drugs, 14(b), 147-51 (1996)).

The albumin (such as human serum albumin) in the composition generally serves as a carrier for the taxane, i.e., the albumin in the composition makes the taxane more readily suspendable in an aqueous medium or helps maintain the suspension as compared to compositions not comprising an albumin. This can avoid the use of toxic solvents (or surfactants) for solubilizing the taxane, and thereby can reduce one or more side effects of administration of the taxane into an individual (such as a human). Thus, in some embodiments, the composition described herein is substantially free (such as free) of surfactants, such as Cremophor (including Cremophor EL® (BASF)). In some embodiments, the nanoparticle composition is substantially free (such as free) of surfactants. A composition is “substantially free of Cremophor” or “substantially free of surfactant” if the amount of Cremophor or surfactant in the composition is not sufficient to cause one or more side effect(s) in an individual when the nanoparticle composition is administered to the individual. In some embodiments, the nanoparticle composition contains less than about any one of 20%, 15%, 10%, 7.5%, 5%, 2.5%, or 1% organic solvent or surfactant.

The amount of albumin in the composition described herein will vary depending on other components in the composition. In some embodiments, the composition comprises an albumin in an amount that is sufficient to stabilize the taxane in an aqueous suspension, for example, in the form of a stable colloidal suspension (such as a stable suspension of nanoparticles). In some embodiments, the albumin is in an amount that reduces the sedimentation rate of the taxane in an aqueous medium. For particle-containing compositions, the amount of the albumin also depends on the size and density of nanoparticles of the taxane.

A taxane is “stabilized” in an aqueous suspension if it remains suspended in an aqueous medium (such as without visible precipitation or sedimentation) for an extended period of time, such as for at least about any of 0.1, 0.2, 0.25, 0.5, 1, 2, 3, 4, 5, 6, 7, 8, 9, 10, 11, 12, 24, 36, 48, 60, or 72 hours. The suspension is generally, but not necessarily, suitable for administration to an individual (such as human). Stability of the suspension is generally (but not necessarily) evaluated at a storage temperature (such as room temperature (such as 20-25° C.) or refrigerated conditions (such as 4° C.)). For example, a suspension is stable at a storage temperature if it exhibits no flocculation or particle agglomeration visible to the naked eye or when viewed under the optical microscope at 1000 times, at about fifteen minutes after preparation of the suspension. Stability can also be evaluated under accelerated testing conditions, such as at a temperature that is higher than about 40° C.

In some embodiments, the albumin is present in an amount that is sufficient to stabilize the taxane in an aqueous suspension at a certain concentration. For example, the concentration of the taxane in the composition is about 0.1 to about 100 mg/ml, including for example any of about 0.1 to about 50 mg/ml, about 0.1 to about 20 mg/ml, about 1 to about 10 mg/ml, about 2 mg/ml to about 8 mg/ml, about 4 to about 6 mg/ml, about 5 mg/ml. In some embodiments, the concentration of the taxane is at least about any of 1.3 mg/ml, 1.5 mg/ml, 2 mg/ml, 3 mg/ml, 4 mg/ml, 5 mg/ml, 6 mg/ml, 7 mg/ml, 8 mg/ml, 9 mg/ml, 10 mg/ml, 15 mg/ml, 20 mg/ml, 25 mg/ml, 30 mg/ml, 40 mg/ml, and 50 mg/ml. In some embodiments, the albumin is present in an amount that avoids use of surfactants (such as Cremophor), so that the composition is free or substantially free of surfactant (such as Cremophor).

In some embodiments, the composition, in liquid form, comprises from about 0.1% to about 50% (w/v) (e.g. about 0.5% (w/v), about 5% (w/v), about 10% (w/v), about 15% (w/v), about 20% (w/v), about 30% (w/v), about 40% (w/v), or about 50% (w/v)) of albumin. In some embodiments, the composition, in liquid form, comprises about 0.5% to about 5% (w/v) of albumin.

In some embodiments, the weight ratio of albumin, e.g., albumin, to the taxane in the nanoparticle composition is such that a sufficient amount of taxane binds to, or is transported by, the cell. While the weight ratio of albumin to taxane will have to be optimized for different albumin and taxane combinations, generally the weight ratio of albumin, e.g., albumin, to taxane (w/w) is about 0.01:1 to about 100:1, about 0.02:1 to about 50:1, about 0.05:1 to about 20:1, about 0.1:1 to about 20:1, about 1:1 to about 18:1, about 2:1 to about 15:1, about 3:1 to about 12:1, about 4:1 to about 10:1, about 5:1 to about 9:1, or about 9:1. In some embodiments, the albumin to taxane weight ratio is about any of 18:1 or less, 15:1 or less, 14:1 or less, 13:1 or less, 12:1 or less, 11:1 or less, 10:1 or less, 9:1 or less, 8:1 or less, 7:1 or less, 6:1 or less, 5:1 or less, 4:1 or less, and 3:1 or less. In some embodiments, the weight ratio of the albumin (such as human serum albumin) and the taxane in the composition is any one of the following: about 1:1 to about 18:1, about 1:1 to about 15:1, about 1:1 to about 12:1, about 1:1 to about 10:1, about 1:1 to about 9:1, about 1:1 to about 8:1, about 1:1 to about 7:1, about 1:1 to about 6:1, about 1:1 to about 5:1, about 1:1 to about 4:1, about 1:1 to about 3:1, about 1:1 to about 2:1, about 1:1 to about 1:1.

In some embodiments, the albumin allows the composition to be administered to an individual (such as human) without significant side effects. In some embodiments, the albumin (such as human serum albumin) is in an amount that is effective to reduce one or more side effects of administration of the taxane to a human. The term “reducing one or more side effects of administration of the taxane” refers to reduction, alleviation, elimination, or avoidance of one or more undesirable effects caused by the taxane, as well as side effects caused by delivery vehicles (such as solvents that render the taxanes suitable for injection) used to deliver the taxane. Such side effects include, for example, myelosuppression, neurotoxicity, hypersensitivity, inflammation, venous irritation, phlebitis, pain, skin irritation, peripheral neuropathy, neutropenic fever, anaphylactic reaction, venous thrombosis, extravasation, and combinations thereof. These side effects, however, are merely exemplary and other side effects, or combination of side effects, associated with taxanes can be reduced.

In some embodiments, the nanoparticle composition comprises ABRAXANE® (Nab-paclitaxel). In some embodiments, the nanoparticle composition is ABRAXANE® (Nab-paclitaxel). ABRAXANE® is a formulation of paclitaxel stabilized by human albumin USP, which can be dispersed in directly injectable physiological solution. When dispersed in a suitable aqueous medium such as 0.9% sodium chloride injection or 5% dextrose injection, ABRAXANE® forms a stable colloidal suspension of paclitaxel. The mean particle size of the nanoparticles in the colloidal suspension is about 130 nanometers. Since HSA is freely soluble in water. ABRAXANE® can be reconstituted in a wide range of concentrations ranging from dilute (0.1 mg/ml paclitaxel) to concentrated (20 mg/ml paclitaxel), including for example about 2 mg/ml to about 8 mg/ml, about 5 mg/ml.

Methods of making nanoparticle compositions are known in the art. For example, nanoparticles containing taxanes (such as paclitaxel) and albumin (such as human serum albumin) can be prepared under conditions of high shear forces (e.g., sonication, high pressure homogenization, or the like). These methods are disclosed in, for example, U.S. Pat. Nos. 5,916,596; 6,506,405; 6,749,868; 6,537,579, 7,820,788, and also in U.S. Pat. Pub. Nos. 2007/0082838, 2006/0263434 and PCT Application WO08/137148.

Briefly, the taxane (such as paclitaxel) is dissolved in an organic solvent, and the solution can be added to an albumin solution. The mixture is subjected to high pressure homogenization. The organic solvent can then be removed by evaporation. The dispersion obtained can be further lyophilized. Suitable organic solvent include, for example, ketones, esters, ethers, chlorinated solvents, and other solvents known in the art. For example, the organic solvent can be methylene chloride or chloroform/ethanol (for example with a ratio of 1:9, 1:8, 1:7, 1:6, 1:5, 1:4, 1:3, 1:2, 1:1, 2:1, 3:1, 4:1, 5:1, 6:1, 7:1, 8:1, or 9:1.

### Methods of Preparation

In some embodiments, there is provided a method of preparing an mRNA or RNAi (e.g., siRNA) delivery complex or nanoparticle as described herein comprising combining a CPP with one or more mRNA, thereby forming the mRNA or RNAi (e.g., siRNA) delivery complex or nanoparticle.

Thus, in some embodiments, there is provided a method of preparing an mRNA or RNAi (e.g., siRNA) delivery complex or nanoparticle as described herein comprising combining a CPP with one or more mRNA.

For example, in some embodiments, there is provided a method of preparing an mRNA or RNAi (e.g., siRNA) delivery complex or nanoparticle as described herein comprising a) combining a first composition comprising one or more mRNA with a second composition comprising a cell-penetrating peptide in an aqueous medium to form a mixture; and b) incubating the mixture to form a complex comprising the cell-penetrating peptide associated with the one or more mRNA, thereby generating the mRNA or RNAi (e.g., siRNA) delivery complex or nanoparticle. In some embodiments, the aqueous medium is a buffer, including for example PBS, Tris, or any buffer known in the art for stabilizing nucleoprotein complexes. In some embodiments, the first composition comprising the one or more mRNA is a solid comprising the one or more mRNA in lyophilized form and a suitable carrier. In some embodiments, the second composition comprising the cell-penetrating peptide is a solution comprising the cell-penetrating peptide at a concentration from about 1 nM to about 200 μM (such as about any of 2 nM, 5 nM, 10 nM, 25 nM, 50 nM, 100 nM, 200 nM, 300 nM, 400 nM, 500 nM, 600 nM, 700 nM, 800 nM, 900 nM, 1 μM, 2 μM, 5 μM, 10 μM, 25 μM, 50 μM, 100 μM, 150 μM, or 200 μM, including any ranges between these values). In some embodiments, the second composition comprising the cell-penetrating peptide is a solid comprising the cell-penetrating peptide in lyophilized form and a suitable carrier. In some embodiments, the solutions are formulated in water. In some embodiments, the water is distilled water. In some embodiments, the solutions are formulated in a buffer. In some embodiments, the buffer is any buffer known in the art used for storing an mRNA or polypeptide. In some embodiments, the molar ratio of the cell-penetrating peptide to mRNA associated with the cell-penetrating peptide in the mixture is between about 1:1 and about 100:1, or between about 1:1 and about 50:1, or about 20:1. In some embodiments, the mixture is incubated to form a complex or nanoparticle comprising the cell-penetrating peptide associated with the one or more mRNA for from about 10 min to 60 min, including for example for about any of 20 min, 30 min, 40 min, and 50 min, at a temperature from about 2° C. to about 50° C., including for example from about 2° C. to about 5° C., from about 5° C. to about 10° C., from about 10° C. to about 15° C., from about 15° C. to about 20° C., from about 20° C. to about 25° C., from about 25° C. to about 30° C., from about 30° C. to about 35° C., from about 35° C. to about 40° C., from about 40° C. to about 45° C., and from about 45° C. to about 50° C., thereby resulting in a solution comprising the mRNA or RNAi (e.g., siRNA) delivery complex or nanoparticle. In some embodiments, the solution comprising the mRNA or RNAi (e.g., siRNA) delivery complex or nanoparticle remains stable for at least about three weeks, including for example for at least about any of 6 weeks, 2 months, 3 months, 4 months, 5 months, and 6 months at 4° C. In some embodiments, the solution comprising the mRNA or RNAi (e.g., siRNA) delivery complex or nanoparticle is lyophilized in the presence of a carrier. In some embodiments, the carrier is a sugar, including for example, sucrose, glucose, mannitol and combinations thereof, and is present in the solution comprising the mRNA or RNAi (e.g., siRNA) delivery complex or nanoparticle at from from about 1% to about 20%, including for example from about 1% to about 10%, from about 10% to 15%, from about 15% to about 20%, weight per volume. In some embodiments, the carrier is a protein, including for example albumin, such as human serum albumin. In some embodiments, the cell-penetrating peptide is a PEP-1 peptide, a PEP-2 peptide, a VEPEP-3 peptide, a VEPEP-6 peptide, a VEPEP-9 peptide, or an ADGN-100 peptide as described herein. In some embodiments, the cell-penetrating peptide comprises the amino acid sequence of any one of SEQ ID NOs: 75-80.

In some embodiments, there is provided a method of preparing a nanoparticle comprising a core and at least one additional layer as described herein, comprising a) combining a composition comprising one or more mRNA with a composition comprising a first cell-penetrating peptide in an aqueous medium to form a first mixture; b) incubating the first mixture to form a core of the nanoparticle comprising the first cell-penetrating peptide associated with the one or more mRNA; c) combining a composition comprising the core of the nanoparticle, such as the mixture of b), with a composition comprising a second cell-penetrating peptide in an aqueous medium to form a second mixture, and d) incubating the second mixture to form a nanoparticle comprising a core and at least one additional layer. In some embodiments, the method further comprises e) combining a composition comprising the nanoparticle comprising a core and at least one additional layer and a composition comprising a third cell-penetrating peptide in an aqueous medium to form a third mixture, and f) incubating the third mixture to form a nanoparticle comprising a core and at least two additional layers. It is to be appreciated that the method can be adapted to form a nanoparticle comprising increasing numbers of layers. In some embodiments, the aqueous medium is a buffer, including for example PBS, Tris, or any buffer known in the art for stabilizing nucleoprotein complexes. In some embodiments, the composition comprising the one or more mRNA is a solution comprising a plurality of mRNA. In some embodiments, the composition comprising the one or more mRNA is a solution further comprising a RNAi (for example, an siRNA). In some embodiments, the composition comprising the one or more mRNA is a solution further comprising a plurality of RNAi (for example, a plurality of RNAi targeting a plurality of genes. In some embodiments, the composition comprising the one or more mRNA is a solid comprising the one or more mRNA in lyophilized form and a suitable carrier. In some embodiments, the compositions comprising the first, second, and/or third cell-penetrating peptides are each a solution comprising the cell-penetrating peptide at a concentration from about 1 nM to about 200 μM (such as about any of 2 nM, 5 nM, 10 nM, 25 nM, 50 nM, 100 nM 200 nM, 300 nM 400 nM, 500 nM, 600 nM 700 nM, 800 nM, 900 nM, 1 μM, 2 μM, 5 μM, 10 μM, 25 μM, 50 μM, 100 μM, 150 μM, or 200 μM, including any ranges between these values). In some embodiments, the compositions comprising the first, second, and/or third cell-penetrating peptides are each a solid comprising the cell-penetrating peptide in lyophilized form and a suitable carrier. In some embodiments, the solutions are formulated in water. In some embodiments, the water is distilled water. In some embodiments, the solutions are formulated in a buffer. In some embodiments, the buffer is any buffer known in the art used for storing an mRNA or polypeptide. In some embodiments, the molar ratio of the first cell-penetrating peptide to mRNA in the first mixture is between about 1:1 and about 100:1, or between about 1:1 and about 50:1, or about 20:1. In some embodiments, the first, second, and/or third mixtures are individually incubated for from about 10 min to 60 min, including for example for about any of 20 min, 30 min, 40 min, and 50 min, at a temperature from about 2° C. to about 50° C., including for example from about 2° C. to about 5° C., from about 5° C. to about 10° C., from about 10° C. to about 15° C., from about 15° C. to about 20° C., from about 20° C. to about 25° C., from about 25° C. to about 30° C., from about 30° C. to about 35° C., from about 35° C. to about 40° C., from about 40° C. to about 45° C., and from about 45° C. to about 50° C. In some embodiments, the solution comprising the nanoparticle remains stable for at least about three weeks, including for example for at least about any of 6 weeks, 2 months, 3 months, 4 months, 5 months, and 6 months at 4° C. In some embodiments, the solution comprising the nanoparticle is lyophilized in the presence of a carrier. In some embodiments, the carrier is a sugar, including for example, sucrose, glucose, mannitol and combinations thereof, and is present in the solution comprising the mRNA or RNAi (e.g., siRNA) delivery complex or nanoparticle at from about 5% to about 20%, including for example from about 7.5% to about 17.5%, from about 10% to about 15%, and about 12.5%, weight per volume. In some embodiments, the carrier is a protein, including for example albumin, such as human serum albumin. In some embodiments, the first, second, and/or third cell-penetrating peptides are individually a PEP-1 peptide, a PEP-2 peptide, a VEPEP-3 peptide, a VEPEP-6 peptide, a VEPEP-9 peptide, or an ADGN-100 peptide as described herein. In some embodiments, the first, second, and/or third cell-penetrating peptides individually comprises the amino acid sequence of SEQ ID NO: 75, 76, 77, 78, 79, or 80.

In some embodiments, the method of preparing a complex, nanoparticle or composition described herein further comprises the step of adding a pharmaceutically acceptable diluent, excipient, and/or carrier (such as a salt, sugar, chemical buffering agent, buffer solution, cell culture medium, or carrier protein) to a composition comprising the complex or nanoparticle, or adjusting the amount of the pharmaceutically acceptable diluent, excipient, and/or carrier in the composition. In some embodiments, the pharmaceutically acceptable diluent, excipient, and/or carrier affects the level of aggregation of an mRNA or RNAi (e.g., siRNA) delivery complex or nanoparticle in the composition and/or the efficiency of intracellular delivery mediated by an mRNA or RNAi (e.g., siRNA) delivery complex or nanoparticle in the composition. In some embodiments, the extent and/or direction of the effect on aggregation and/or delivery efficiency mediated by the pharmaceutically acceptable diluent, excipient, and/or carrier is dependent on the relative amount of the pharmaceutically acceptable diluent, excipient, and/or carrier in the composition.

For example, in some embodiments, the method of preparing an mRNA or RNAi (e.g., siRNA) delivery complex, nanoparticle, or composition described herein further comprises the step of adding to a composition comprising the mRNA or RNAi (e.g., siRNA) delivery complex or nanoparticle a pharmaceutically acceptable diluent, excipient, and/or carrier, or adjusting the composition, to arrive at a concentration of the pharmaceutically acceptable diluent, excipient, and/or carrier that does not promote and/or contribute to aggregation of the mRNA or RNAi (e.g., siRNA) delivery complex or nanoparticle, or promotes and/or contributes to the formation of aggregates of the mRNA or RNAi (e.g., siRNA) delivery complex or nanoparticles having a size no more than about 200% (such as no more than about any of 190, 180, 170, 160, 150, 140, 130, 120, 110, 100, 90, 80, 70, 60, 50, 40, 30, 20, 10, 9, 8, 7, 6, 5, 4, 3, 2, or 1%, including any ranges between any of these values) larger than the size of the mRNA or RNAi (e.g., siRNA) delivery complex or nanoparticle. In some embodiments, the pharmaceutically acceptable diluent, excipient, and/or carrier is added to the composition, or the composition is adjusted, to arrive at a concentration of the pharmaceutically acceptable diluent, excipient, and/or carrier in the composition that promotes and/or contributes to the formation of aggregates of the mRNA or RNAi (e.g., siRNA) delivery complex or nanoparticles having a size no more than about 150% larger than the size of the mRNA or RNAi (e.g., siRNA) delivery complex or nanoparticle. In some embodiments, the pharmaceutically acceptable diluent, excipient, and/or carrier is added to the composition, or the composition is adjusted, to arrive at a concentration of the pharmaceutically acceptable diluent, excipient, and/or carrier in the composition that promotes and/or contributes to the formation of aggregates of the mRNA or RNAi (e.g., siRNA) delivery complex or nanoparticles having a size no more than about 100% larger than the size of the mRNA or RNAi (e.g., siRNA) delivery complex or nanoparticle. In some embodiments, the pharmaceutically acceptable diluent, excipient, and/or carrier is added to the composition, or the composition is adjusted, to arrive at a concentration of the pharmaceutically acceptable diluent, excipient, and/or carrier in the composition that promotes and/or contributes to the formation of aggregates of the mRNA or RNAi (e.g., siRNA) delivery complex or nanoparticles having a size no more than about 50% larger than the size of the mRNA or RNAi (e.g., siRNA) delivery complex or nanoparticle. In some embodiments, the pharmaceutically acceptable diluent, excipient, and/or carrier is added to the composition, or the composition is adjusted, to arrive at a concentration of the pharmaceutically acceptable diluent, excipient, and/or carrier in the composition that promotes and/or contributes to the formation of aggregates of the mRNA or RNAi (e.g., siRNA) delivery complex or nanoparticles having a size no more than about 20% larger than the size of the mRNA or RNAi (e.g., siRNA) delivery complex or nanoparticle. In some embodiments, the pharmaceutically acceptable diluent, excipient, and/or carrier is added to the composition, or the composition is adjusted, to arrive at a concentration of the pharmaceutically acceptable diluent, excipient, and/or carrier in the composition that promotes and/or contributes to the formation of aggregates of the mRNA or RNAi (e.g., siRNA) delivery complex or nanoparticles having a size no more than about 15% larger than the size of the mRNA or RNAi (e.g., siRNA) delivery complex or nanoparticle. In some embodiments, the pharmaceutically acceptable diluent, excipient, and/or carrier is added to the composition, or the composition is adjusted, to arrive at a concentration of the pharmaceutically acceptable diluent, excipient, and/or carrier in the composition that promotes and/or contributes to the formation of aggregates of the mRNA or RNAi (e.g., siRNA) delivery complex or nanoparticles having a size no more than about 10% larger than the size of the mRNA or RNAi (e.g., siRNA) delivery complex or nanoparticle. In some embodiments, the pharmaceutically acceptable diluent, excipient, and/or carrier is a salt, including, without limitation, NaCl. In some embodiments, the pharmaceutically acceptable diluent, excipient, and/or carrier is a sugar, including, without limitation, sucrose, glucose, and mannitol. In some embodiments, the pharmaceutically acceptable diluent, excipient, and/or carrier is a chemical buffering agent, including, without limitation, HEPES. In some embodiments, the pharmaceutically acceptable diluent, excipient, and/or carrier is a buffer solution, including, without limitation, PBS. In some embodiments, the pharmaceutically acceptable diluent, excipient, and/or carrier is a cell culture medium, including, without limitation, DMEM. Particle size can be determined using any means known in the art for measuring particle size, such as by dynamic light scattering (DLS). For example, in some embodiments, an aggregate having a Z-average as measured by DLS that is 10% greater than the Z-average as measured by DLS of an mRNA or RNAi (e.g., siRNA) delivery complex or nanoparticle is 10% larger than the mRNA or RNAi (e.g., siRNA) delivery complex or nanoparticle.

In some embodiments, the method of preparing an mRNA or RNAi (e.g., siRNA) delivery complex, nanoparticle, or composition described herein further comprises the step of adding to a composition comprising the mRNA or RNAi (e.g., siRNA) delivery complex or nanoparticle a salt (e.g., NaCl), or adjusting the composition, to arrive at a concentration of the salt in the composition that does not promote and/or contribute to aggregation of the mRNA or RNAi (e.g., siRNA) delivery complex or nanoparticle, or promotes and/or contributes to the formation of aggregates of the mRNA or RNAi (e.g., siRNA) delivery complex or nanoparticles having a size no more than about 100% (such as no more than about any of 90, 80, 70, 60, 50, 40, 30, 20, 10, 9, 8, 7, 6, 5, 4, 3, 2, or %, including any ranges between any of these values) larger than the size of the mRNA or RNAi (e.g., siRNA) delivery complex or nanoparticle. In some embodiments, the salt (e.g., NaCl) is added to the composition, or the composition is adjusted, to arrive at a concentration of the salt (e.g., NaCl) in the composition that promotes and/or contributes to the formation of aggregates of the mRNA or RNAi (e.g., siRNA) delivery complex or nanoparticles having a size no more than about 75% larger than the size of the mRNA or RNAi (e.g., siRNA) delivery complex or nanoparticle. In some embodiments, the salt (e.g., NaCl) is added to the composition, or the composition is adjusted, to arrive at a concentration of the salt (e.g., NaCl) in the composition that promotes and/or contributes to the formation of aggregates of the mRNA or RNAi (e.g., siRNA) delivery complex or nanoparticles having a size no more than about 50% larger than the size of the mRNA or RNAi (e.g., siRNA) delivery complex or nanoparticle. In some embodiments, the salt (e.g., NaCl) is added to the composition, or the composition is adjusted, to arrive at a concentration of the salt (e.g., NaCl) in the composition that promotes and/or contributes to the formation of aggregates of the mRNA or RNAi (e.g., siRNA) delivery complex or nanoparticles having a size no more than about 20% larger than the size of the mRNA or RNAi (e.g., siRNA) delivery complex or nanoparticle. In some embodiments, the salt (e.g., NaCl) is added to the composition, or the composition is adjusted, to arrive at a concentration of the salt (e.g., NaCl) in the composition that promotes and/or contributes to the formation of aggregates of the mRNA or RNAi (e.g., siRNA) delivery complex or nanoparticles having a size no more than about 15% larger than the size of the mRNA or RNAi (e.g., siRNA) delivery complex or nanoparticle. In some embodiments, the salt (e.g., NaCl) is added to the composition, or the composition is adjusted, to arrive at a concentration of the salt (e.g., NaCl) in the composition that promotes and/or contributes to the formation of aggregates of the mRNA or RNAi (e.g., siRNA) delivery complex or nanoparticles having a size no more than about 10% larger than the size of the mRNA or RNAi (e.g., siRNA) delivery complex or nanoparticle. In some embodiments, the concentration of the salt in the composition is no more than about 100 mM (such as no more than about any of 90, 80, 70, 60, 50, 40, 30, 20, 10, 9, 8, 7, 6, 5, 4, 3, 2, or 1 mM, including any ranges between any of these values). In some embodiments, the salt is NaCl.

In some embodiments, the method of preparing an mRNA or RNAi (e.g., siRNA) delivery complex, nanoparticle, or composition described herein further comprises the step of adding to a composition comprising the mRNA or RNAi (e.g., siRNA) delivery complex or nanoparticle a sugar (e.g., sucrose, glucose, or mannitol), or adjusting the composition, to arrive at a concentration of the sugar in the composition that does not promote and/or contribute to aggregation of the mRNA or RNAi (e.g., siRNA) delivery complex or nanoparticle, or promotes and/or contributes to the formation of aggregates of the mRNA or RNAi (e.g., siRNA) delivery complex or nanoparticles having a size no more than about 25% (such as no more than about any of 24, 23, 22, 21, 20, 19, 18, 17, 16, 15, 14, 13, 12, 11, 10, 9, 8, 7, 6, 5, 4, 3, 2, or 1%, including any ranges between any of these values) larger than the size of the mRNA or RNAi (e.g., siRNA) delivery complex or nanoparticle. In some embodiments, the sugar (e.g., sucrose, glucose, or mannitol) is added to the composition, or the composition is adjusted, to arrive at a concentration of the sugar (e.g., sucrose, glucose, or mannitol) in the composition that promotes and/or contributes to the formation of aggregates of the mRNA or RNAi (e.g., siRNA) delivery complex or nanoparticles having a size no more than about 75% larger than the size of the mRNA or RNAi (e.g., siRNA) delivery complex or nanoparticle. In some embodiments, the sugar (e.g., sucrose, glucose, or mannitol) is added to the composition, or the composition is adjusted, to arrive at a concentration of the sugar (e.g., sucrose, glucose, or mannitol) in the composition that promotes and/or contributes to the formation of aggregates of the mRNA or RNAi (e.g., siRNA) delivery complex or nanoparticles having a size no more than about 50% larger than the size of the mRNA or RNAi (e.g., siRNA) delivery complex or nanoparticle. In some embodiments, the sugar (e.g., sucrose, glucose, or mannitol) is added to the composition, or the composition is adjusted, to arrive at a concentration of the sugar (e.g., sucrose, glucose, or mannitol) in the composition that promotes and/or contributes to the formation of aggregates of the mRNA or RNAi (e.g., siRNA) delivery complex or nanoparticles having a size no more than about 20% larger than the size of the mRNA or RNAi (e.g., siRNA) delivery complex or nanoparticle. In some embodiments, the sugar (e.g., sucrose, glucose, or mannitol) is added to the composition, or the composition is adjusted, to arrive at a concentration of the sugar (e.g., sucrose, glucose, or mannitol) in the composition that promotes and/or contributes to the formation of aggregates of the mRNA or RNAi (e.g., siRNA) delivery complex or nanoparticles having a size no more than about 15% larger than the size of the mRNA or RNAi (e.g., siRNA) delivery complex or nanoparticle. In some embodiments, the sugar (e.g., sucrose, glucose, or mannitol) is added to the composition, or the composition is adjusted, to arrive at a concentration of the sugar (e.g., sucrose, glucose, or mannitol) in the composition that promotes and/or contributes to the formation of aggregates of the mRNA or RNAi (e.g., siRNA) delivery complex or nanoparticles having a size no more than about 10% larger than the size of the mRNA or RNAi (e.g., siRNA) delivery complex or nanoparticle. In some embodiments, the concentration of the sugar in the composition is no more than about 20% (such as no more than about any of 18, 16, 14, 12, 10, 9, 8, 7, 6, 5, 4, 3, 2, or 1%, including any ranges between any of these values). In some embodiments, the sugar is sucrose. In some embodiments, the sugar is glucose. In some embodiments, the sugar is mannitol.

In some embodiments, the method of preparing an mRNA or RNAi (e.g., siRNA) delivery complex, nanoparticle, or composition described herein further comprises the step of adding to a composition comprising the mRNA or RNAi (e.g., siRNA) delivery complex or nanoparticle a chemical buffering agent (e.g., HEPES or phosphate), or adjusting the composition, to arrive at a concentration of the chemical buffering agent in the composition that does not promote and/or contribute to aggregation of the mRNA or RNAi (e.g., siRNA) delivery complex or nanoparticle, or promotes and/or contributes to the formation of aggregates of the mRNA or RNAi (e.g., siRNA) delivery complex or nanoparticles having a size no more than about 10% (such as no more than about any of 9, 8, 7, 6, 5, 4, 3, 2, or 1%, including any ranges between any of these values) larger than the size of the mRNA or RNAi (e.g., siRNA) delivery complex or nanoparticle. In some embodiments, the chemical buffering agent (e.g., HEPES or phosphate) is added to the composition, or the composition is adjusted, to arrive at a concentration of the chemical buffering agent (e.g., HEPES or phosphate) in the composition that promotes and/or contributes to the formation of aggregates of the mRNA or RNAi (e.g., siRNA) delivery complex or nanoparticles having a size no more than about 7.5% larger than the size of the mRNA or RNAi (e.g., siRNA) delivery complex or nanoparticle. In some embodiments, the chemical buffering agent (e.g., HEPES or phosphate) is added to the composition, or the composition is adjusted, to arrive at a concentration of the chemical buffering agent (e.g., HEPES or phosphate) in the composition that promotes and/or contributes to the formation of aggregates of the mRNA or RNAi (e.g., siRNA) delivery complex or nanoparticles having a size no more than about 5% larger than the size of the mRNA or RNAi (e.g., siRNA) delivery complex or nanoparticle. In some embodiments, the chemical buffering agent (e.g., HEPES or phosphate) is added to the composition, or the composition is adjusted, to arrive at a concentration of the chemical buffering agent (e.g., HEPES or phosphate) in the composition that promotes and/or contributes to the formation of aggregates of the mRNA or RNAi (e.g., siRNA) delivery complex or nanoparticles having a size no more than about 3% larger than the size of the mRNA or RNAi (e.g., siRNA) delivery complex or nanoparticle. In some embodiments, the chemical buffering agent (e.g., HEPES or phosphate) is added to the composition, or the composition is adjusted, to arrive at a concentration of the chemical buffering agent (e.g., HEPES or phosphate) in the composition that promotes and/or contributes to the formation of aggregates of the mRNA or RNAi (e.g., siRNA) delivery complex or nanoparticles having a size no more than about 1% larger than the size of the mRNA or RNAi (e.g., siRNA) delivery complex or nanoparticle. In some embodiments, the chemical buffering agent (e.g., HEPES or phosphate) is added to the composition, or the composition is adjusted, to arrive at a concentration of the chemical buffering agent (e.g., HEPES or phosphate) in the composition that does not promote and/or contribute to the formation of aggregates of the mRNA or RNAi (e.g., siRNA) delivery complex or nanoparticles. In some embodiments, the chemical buffering agent is HEPES. In some embodiments, the HEPES is added to the composition in the form of a buffer solution comprising HEPES. In some embodiments, the solution comprising HEPES has a pH between about 5 and about 9 (such as about any of 5, 5.5, 6, 6.5, 7, 7.5, 8, 8.5, and 9, including any ranges between these values). In some embodiments, the composition comprises HEPES at a concentration of no more than about 75 mM (such as no more than about any of 70, 65, 60, 55, 50, 45, 40, 35, 30, 25, 20, 15, 10 mM or less, including any ranges between any of these values). In some embodiments, the chemical buffering agent is phosphate. In some embodiments, the phosphate is added to the composition in the form of a buffer solution comprising phosphate. In some embodiments, the composition does not comprise PBS.

In some embodiments, the method of preparing an mRNA or RNAi (e.g., siRNA) delivery complex, nanoparticle, or composition described herein further comprises the step of adding to a composition comprising the mRNA or RNAi (e.g., siRNA) delivery complex or nanoparticle a cell culture medium (e.g., DMEM or Opti-MEM), or adjusting the composition, to arrive at a concentration of the cell culture medium in the composition that does not promote and/or contribute to aggregation of the mRNA or RNAi (e.g., siRNA) delivery complex or nanoparticle, or promotes and/or contributes to the formation of aggregates of the mRNA or RNAi (e.g., siRNA) delivery complex or nanoparticles having a size no more than about 200% (such as no more than about any of 190, 180, 170, 160, 150, 140, 130, 120, 110, 100, 90, 80, 70, 60, 50, 40, 30, 20, 10, 9, 8, 7, 6, 5, 4, 3, 2, or 1%, including any ranges between any of these values) larger than the size of the mRNA or RNAi (e.g., siRNA) delivery complex or nanoparticle. In some embodiments, the cell culture medium (e.g., DMEM or Opti-MEM) is added to the composition, or the composition is adjusted, to arrive at a concentration of the cell culture medium (e.g., DMEM or Opti-MEM) in the composition that promotes and/or contributes to the formation of aggregates of the mRNA or RNAi (e.g., siRNA) delivery complex or nanoparticles having a size no more than about 150% larger than the size of the mRNA or RNAi (e.g., siRNA) delivery complex or nanoparticle. In some embodiments, the cell culture medium (e.g., DMEM or Opti-MEM) is added to the composition, or the composition is adjusted, to arrive at a concentration of the cell culture medium (e.g., DMEM or Opti-MEM) in the composition that promotes and/or contributes to the formation of aggregates of the mRNA or RNAi (e.g., siRNA) delivery complex or nanoparticles having a size no more than about 100% larger than the size of the mRNA or RNAi (e.g., siRNA) delivery complex or nanoparticle. In some embodiments, the cell culture medium (e.g., DMEM or Opti-MEM) is added to the composition, or the composition is adjusted, to arrive at a concentration of the cell culture medium (e.g., DMEM or Opti-MEM) in the composition that promotes and/or contributes to the formation of aggregates of the mRNA or RNAi (e.g., siRNA) delivery complex or nanoparticles having a size no more than about 50% larger than the size of the mRNA or RNAi (e.g., siRNA) delivery complex or nanoparticle. In some embodiments, the cell culture medium (e.g., DMEM or Opti-MEM) is added to the composition, or the composition is adjusted, to arrive at a concentration of the cell culture medium (e.g., DMEM or Opti-MEM) in the composition that promotes and/or contributes to the formation of aggregates of the mRNA or RNAi (e.g., siRNA) delivery complex or nanoparticles having a size no more than about 25% larger than the size of the mRNA or RNAi (e.g., siRNA) delivery complex or nanoparticle. In some embodiments, the cell culture medium (e.g., DMEM or Opti-MEM) is added to the composition, or the composition is adjusted, to arrive at a concentration of the cell culture medium (e.g., DMEM or Opti-MEM) in the composition that promotes and/or contributes to the formation of aggregates of the mRNA or RNAi (e.g., siRNA) delivery complex or nanoparticles having a size no more than about 10% larger than the size of the mRNA or RNAi (e.g., siRNA) delivery complex or nanoparticle. In some embodiments, the composition comprises the cell culture medium at a concentration of no more than about 70% (such as no more than about any of 65, 60, 55, 50, 45, 40, 35, 30, 25, 20, 15, 10%, or less, including any ranges between any of these values). In some embodiments, the cell culture medium is DMEM. In some embodiments, the cell culture medium is Opti-MEM.

In some embodiments, the method of preparing an mRNA or RNAi (e.g., siRNA) delivery complex, nanoparticle, or composition described herein further comprises the step of adding to a composition comprising the mRNA or RNAi (e.g., siRNA) delivery complex or nanoparticle a carrier protein (e.g., albumin), or adjusting the composition, to arrive at a concentration of the carrier protein in the composition that does not promote and/or contribute to aggregation of the mRNA or RNAi (e.g., siRNA) delivery complex or nanoparticle, or promotes and/or contributes to the formation of aggregates of the mRNA or RNAi (e.g., siRNA) delivery complex or nanoparticles having a size no more than about 200% (such as no more than about any of 190, 180, 170, 160, 150, 140, 130, 120, 110, 100, 90, 80, 70, 60, 50, 40, 30, 20, 10, 9, 8, 7, 6, 5, 4, 3, 2, or 1%, including any ranges between any of these values) larger than the size of the mRNA or RNAi (e.g., siRNA) delivery complex or nanoparticle. In some embodiments, the carrier protein (e.g., albumin) is added to the composition, or the composition is adjusted, to arrive at a concentration of the carrier protein (e.g., albumin) in the composition that promotes and/or contributes to the formation of aggregates of the mRNA or RNAi (e.g., siRNA) delivery complex or nanoparticles having a size no more than about 150% larger than the size of the mRNA or RNAi (e.g., siRNA) delivery complex or nanoparticle. In some embodiments, the carrier protein (e.g., albumin) is added to the composition, or the composition is adjusted, to arrive at a concentration of the carrier protein (e.g., albumin) in the composition that promotes and/or contributes to the formation of aggregates of the mRNA or RNAi (e.g., siRNA) delivery complex or nanoparticles having a size no more than about 100% larger than the size of the mRNA or RNAi (e.g., siRNA) delivery complex or nanoparticle. In some embodiments, the carrier protein (e.g., albumin) is added to the composition, or the composition is adjusted, to arrive at a concentration of the carrier protein (e.g., albumin) in the composition that promotes and/or contributes to the formation of aggregates of the mRNA or RNAi (e.g., siRNA) delivery complex or nanoparticles having a size no more than about 50% larger than the size of the mRNA or RNAi (e.g., siRNA) delivery complex or nanoparticle. In some embodiments, the carrier protein (e.g., albumin) is added to the composition, or the composition is adjusted, to arrive at a concentration of the carrier protein (e.g., albumin) in the composition that promotes and/or contributes to the formation of aggregates of the mRNA or RNAi (e.g., siRNA) delivery complex or nanoparticles having a size no more than about 25% larger than the size of the mRNA or RNAi (e.g., siRNA) delivery complex or nanoparticle. In some embodiments, the carrier protein (e.g., albumin) is added to the composition, or the composition is adjusted, to arrive at a concentration of the carrier protein (e.g., albumin) in the composition that promotes and/or contributes to the formation of aggregates of the mRNA or RNAi (e.g., siRNA) delivery complex or nanoparticles having a size no more than about 10% larger than the size of the mRNA or RNAi (e.g., siRNA) delivery complex or nanoparticle. In some embodiments, the carrier protein is albumin. In some embodiments, the albumin is human serum albumin.

In some embodiments, for a stable composition comprising an mRNA or RNAi (e.g., siRNA) delivery complex or nanoparticle of the invention, the average diameter of the complex or nanoparticle does not change by more than about 10%, and the polydispersity index does not change by more than about 10%.

### Methods of Use

### Methods of Disease Treatment

The present invention in one aspect provides methods of treating a disease or condition in an individual comprising delivering to the individual an mRNA and/or a RNAi (e.g., siRNA). In some embodiments, there is provided a method of treating a disease or condition in an individual comprising administering to the individual an effective amount of a pharmaceutical composition comprising an mRNA or RNAi (e.g., siRNA) delivery complex or nanoparticle as described herein for intracellular delivery of an mRNA and a pharmaceutically acceptable carrier, wherein the mRNA or RNAi (e.g., siRNA) delivery complex or nanoparticle comprises one or more mRNA useful for the treatment of the disease or condition. In some embodiments, the mRNA is modified (e.g., wherein at least one modified nucleoside is 5-methoxyuridine (5moU)). In some embodiments, the mRNA or RNAi (e.g., siRNA) delivery complex or nanoparticle comprises a CPP comprising the amino acid sequence of a PEP-1 peptide, a PEP-2 peptide, a VEPEP-3 peptide, a VEPEP-6 peptide, a VEPEP-9 peptide, or an ADGN-100 peptide. In some embodiments, the lowest effective amount of mRNA in the pharmaceutical composition is less than the lowest effective amount of mRNA in a similar pharmaceutical composition where the mRNA is not in an mRNA or RNAi (e.g., siRNA) delivery complex or nanoparticle as described herein (e.g., a pharmaceutical composition comprising free mRNA). In some embodiments, the mRNA encodes a therapeutic protein, for example, a tumor suppressor protein. In some embodiments, the mRNA or RNAi (e.g., siRNA) delivery complex or nanoparticle as described herein further comprises an inhibitory RNA (RNAi), such as an RNAi targeting an endogenous gene, e.g., a disease-associated endogenous gene. In some embodiments, the RNAi targets an exogenous gene. In some embodiments, the complex or nanoparticle comprises one or more mRNA comprising a first mRNA encoding a first therapeutic protein, and a second mRNA encoding a second therapeutic protein. In some embodiments, the complex or nanoparticle comprises a plurality of RNAi (for example, siRNA and/or a microRNA), wherein the plurality of RNAi targets a plurality of endogenous genes involved in a disease or condition. In some embodiments, the complex of nanoparticle comprises a therapeutic mRNA and a therapeutic RNAi, wherein the therapeutic mRNA encodes a therapeutic protein, and wherein the therapeutic RNAi targets an endogenous gene involved in a disease or condition. In some embodiments, the therapeutic RNAi targets a disease-associated form of the endogenous gene (e.g., a gene encoding a mutant protein, or a gene resulting in abnormal expression of a protein), and mRNA is a therapeutic form of the endogenous gene (e.g., the second transgene encodes a wild-type or functional form of the mutant protein, or the second transgene results in normal expression of the protein). In some embodiments, the complex or nanoparticle comprises a first mRNA encoding the first therapeutic protein and a second mRNA encoding a second therapeutic mRNA. In some embodiments, the complex or nanoparticle comprises a single mRNA encoding a plurality of proteins. In some embodiments, the disease or condition to be treated includes, but is not limited to, cancer, diabetes, autoimmune diseases, inflammatory diseases, fibrotic diseases, viral infectious diseases, hereditary diseases, ocular diseases, aging and degenerative diseases, and diseases characterized by cholesterol level abnormality. In some embodiments, the mRNA is capable of modulating the expression of one or more genes. In some embodiments, the one or more genes encode proteins including, but not limited to, growth factors and cytokines, cell surface receptors, signaling molecules and kinases, transcription factors and other modulators of transcription, regulators of protein expression and modification, tumor suppressors, and regulators of apoptosis and metastasis. In some embodiments, the pharmaceutical composition further comprises one or more additional mRNA or RNAi (e.g., siRNA) delivery complexes or nanoparticles as described herein. In some embodiments, the method further comprises administering to the individual an effective amount of one or more additional pharmaceutical compositions comprising one or more additional mRNA or RNAi (e.g., siRNA) delivery complexes or nanoparticles as described herein.

“Modulation” of activity or expression used herein means regulating or altering the status or copy numbers of a gene or mRNA or changing the amount of gene product such as a protein that is produced. In some embodiments, the mRNA and/or RNAi increases the expression of a target gene. In some embodiments, the mRNA increases the expression of the gene or gene product by at least about any of 0%, 20%, 30%, 40%, 60%, 70%, 80%, 90%, or 100%. In some embodiments, the mRNA and/or RNAi inhibits the expression of a target gene. In some embodiments, the mRNA inhibits the expression of the gene or gene product by at least about any of 0%, 20%, 30%, 40%, 60%, 70%, 80%, 90%, or 100%.

In some embodiments, there is provided a method of treating a disease or condition in an individual comprising administering to the individual an effective amount of a pharmaceutical composition comprising an mRNA or RNAi (e.g., siRNA) delivery complex or nanoparticle as described herein for intracellular delivery of an mRNA and a pharmaceutically acceptable carrier, wherein the mRNA or RNAi (e.g., siRNA) delivery complex or nanoparticle comprises one or more mRNA useful for the treatment of the disease or condition and a cell-penetrating peptide comprising the amino acid sequence of a PEP-1 peptide, a PEP-2 peptide, a VEPEP-3 peptide, a VEPEP-6 peptide, a VEPEP-9 peptide, or an ADGN-100 peptide. In some embodiments, the VEPEP-3 peptide comprises the amino acid sequence of any one of SEQ ID NOs: 1-14, 75, and 76. In some embodiments, the VEPEP-6 peptide comprises the amino acid sequence of any one of SEQ ID NOs: 15-40, and 77. In some embodiments, the VEPEP-9 peptide comprises the amino acid sequence of any one of SEQ ID NOs: 41-52, and 78. In some embodiments, the ADGN-100 peptide comprises the amino acid sequence of any one of SEQ ID NOs: 53-70, 79, and 80. In some embodiments, the disease or condition to be treated includes, but is not limited to, cancer, diabetes, autoimmune diseases, inflammatory diseases, fibrotic diseases, viral infectious diseases, hereditary diseases, ocular diseases, aging and degenerative diseases, and cholesterol level abnormality. In some embodiments, the mRNA or RNAi (e.g., siRNA) delivery complex or nanoparticle in the pharmaceutical composition comprises one or more mRNA for modulating the expression of one or more genes in the individual. In some embodiments, the one or more genes encode proteins including, but not limited to, growth factors and cytokines, cell surface receptors, signaling molecules and kinases, transcription factors and other modulators of transcription, regulators of protein expression and modification, tumor suppressors, and regulators of apoptosis and metastasis. In some embodiments, the pharmaceutical composition further comprises one or more additional mRNA or RNAi (e.g., siRNA) delivery complexes or nanoparticles as described herein. In some embodiments, the method further comprises administering to the individual an effective amount of one or more additional pharmaceutical compositions comprising one or more additional mRNA or RNAi (e.g., siRNA) delivery complexes or nanoparticles as described herein.

In some embodiments of the methods described herein, the mRNA or RNAi (e.g., siRNA) delivery complex or nanoparticle comprises one or more mRNA encoding one or more protein, such as one or more therapeutic protein. In some embodiments, one or more mRNA encode a chimeric antigen receptor (CAR). In some embodiments, the mRNA or RNAi (e.g., siRNA) delivery complex or nanoparticle further comprises inhibitory RNA (RNAi), such as a therapeutic RNAi.

In some embodiments, there is provided a method of treating a disease or condition in an individual comprising administering to the individual an effective amount of a pharmaceutical composition comprising an mRNA or RNAi (e.g., siRNA) delivery complex or nanoparticle as described herein and a pharmaceutically acceptable carrier, wherein the method comprises multiple administrations of the pharmaceutical composition. In some embodiments, repeated administrations of the pharmaceutical compositions do not elicit an adverse immune response in the individual to the pharmaceutical composition, or elicit a substantially reduced immune response in the individual compared to repeated administrations of a similar pharmaceutical composition comprising the one or more mRNA contained in the mRNA or RNAi (e.g., siRNA) delivery complex or nanoparticle alone. In some embodiments, a repeated administration of the pharmaceutical compositions results in an immune response no more than about 99% (such as no more than about any of 95, 90, 85, 80, 75, 70, 65, 60, 55, 50, 45, 40, 35, 30, 25, 20, 15, 10, 5. 4, 3, 2, 1% or less, including any ranges between these values) as strong as the immune response generated by a corresponding repeated administration of a similar pharmaceutical composition comprising the one or more mRNA contained in the mRNA or RNAi (e.g., siRNA) delivery complex or nanoparticle alone.

In some embodiments, there is provided a method of treating a disease or condition in an individual comprising administering to the individual an effective amount of a pharmaceutical composition comprising an mRNA or RNAi (e.g., siRNA) delivery complex or nanoparticle as described herein and a pharmaceutically acceptable carrier, wherein the complex or nanoparticle is delivered to a local tissue, organ or cell. In some embodiments, there is provided a method of treating a disease or condition in an individual comprising administering to the individual an effective amount of a pharmaceutical composition comprising an mRNA or RNAi (e.g., siRNA) delivery complex or nanoparticle as described herein and a pharmaceutically acceptable carrier, wherein the complex or nanoparticle is delivered to a blood vessel or a tissue surrounding blood vessel.

### Diseases and Conditions

In some embodiments of the methods described herein, the disease to be treated is cancer. In some embodiments, the cancer is a solid tumor, and the pharmaceutical composition comprises an mRNA delivery complex or nanoparticle comprising one or more mRNA that encode proteins including, but not limited to, growth factors and cytokines, cell surface receptors, signaling molecules and kinases, transcription factors and other modulators of transcription, regulators of protein expression and modification, tumor suppressors, and regulators of apoptosis and metastasis. In some embodiments, the growth factors or cytokines include, but are not limited to, EGF, VEGF, FGF, HGF, HDGF, IGF, PDGF, TGF-α, TGF-β, TNF-α, and wnt, including mutants thereof. In some embodiments, the cell surface receptors include, but are not limited to, ER, PR, Her2, Her3, angiopoietin receptor, EGFR, FGFR, HGFR, HDGFR, IGFR, KGFR, MSFR, PDGFR, TGFR, VEGFR1, VEGFR2, VEGFR3, Frizzled family receptors (FZD-1 to 10), smoothened, patched, and CXCR4, including mutants thereof. In some embodiments, the signaling molecules or kinases include, but are not limited to, KRAS, NRAS, RAF, MEK, MEKK, MAPK, MKK, ERK, JNK, JAK, PKA, PKC, PI3K, Akt, mTOR, Raptor, Rictor, MLST8, PRAS40, DEPTOR, MSIN1, S6 kinase, PDK1, BRAF, FAK, Src, Fyn, Shc, GSK, IKK, PLK-1, cyclin-dependent kinases (Cdk1 to 13), CDK-activating kinases, ALK/Met, Syk, BTK, Bcr-Abl, RET, β-catenin, Mcl-1, and PKN3, including mutants thereof. In some embodiments, the transcription factors or other modulators of transcription include, but are not limited to, AR, ATF1, CEBPA, CREB1, ESR1, EWSR1, FOXO1, GATA1, GATA3, HNF1A, HNF1B, IKZF1, IRF1, IRF4, KLF6, LMO1, LYL1, MYC, NR4A3, PAX3, PAX5, PAX7, PBX1, PHOX2B, PML, RUNX1, SMAD4, SMAD7, STAT5B, TAL1, TP53, WT1, ZBTB16, ATF-2, Chop, c-Jun, c-Myc, DPC4, Elk-1, Ets1, Max, MEF2C, NFAT4, Sap1a, STATs, Ta1, p53, CREB, NF-κB, HDACs, HIF-1α, and RRM2, including mutants thereof. In some embodiments, the regulators of protein expression or modification include, but are not limited to, ubiquitin ligase, LMP2, LMP7, and MECL-1, including mutants thereof. In some embodiments, the tumor suppressors include, but are not limited to, APC, BRCA1, BRCA2, DPC4, INK4, MADR2, MLH1, MSH2, MSH6, NF, NF2, p53, PTC, PTEN, Rb, VHL, WT1, WT2, and components of SWI/SNF chromatin remodeling complex including mutants thereof. In some embodiments, the regulators of apoptosis or metastasis include, but are not limited to, XIAP, Bcl-2, osteopontin, SPARC, MMP-2, MMP-9, uPAR, including mutants thereof.

In some embodiments, the solid tumor includes, but is not limited to, sarcomas and carcinomas such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, Kaposi's sarcoma, soft tissue sarcoma, uterine sacronomasynovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma, and retinoblastoma.

In some embodiments, the mRNA delivery complex or nanoparticle further comprises a RNAi (such as siRNA) that targets an endogenous gene, e.g., a disease-associated endogenous gene, for example, an oncogene. In some embodiments, the oncogene is rasK. In some embodiments, the oncogene is KRAS. In some embodiments, the RNAi targets an exogenous gene.

In some embodiments of the methods described herein, the disease to be treated is cancer, wherein the cancer is a solid tumor, and the pharmaceutical composition comprises an mRNA delivery complex or nanoparticle comprising one or more mRNA encoding proteins involved in tumor development and/or progression. In some embodiments, the mRNA encodes proteins involved in tumor development and/or progression include, but are not limited to, IL-2, IL-12, interferon-gamma, GM-CSF, B7-1, caspase-9, p53, MUC-1, MDR-1, HLA-B7/Beta 2-Microglobulin, Her2, Hsp27, thymidine kinase, and MDA-7, including mutants thereof. In some embodiments, the mRNA encodes a protein, such as a therapeutic protein. In some embodiments, mRNA encodes a CAR In some embodiments, the complex or nanoparticle comprises a plurality of mRNA encoding a plurality of protein. In some embodiments, the complex or nanoparticle comprises a plurality of mRNA encoding a single protein. In some embodiments, the complex or nanoparticle comprises a single mRNA encoding a first protein and a second protein. In some embodiments, the complex or nanoparticle further comprises a RNAi such as siRNA, such as an RNAi targeting an endogenous gene, e.g., a disease-associated endogenous gene. In some embodiments, the RNAi targets an exogenous gene. In some embodiments, the RNAi is a therapeutic RNAi targeting an endogenous gene involved in a disease or condition, and the protein is a therapeutic protein useful for treating the disease or condition. In some embodiments, the complex or nanoparticle comprises a therapeutic mRNA and a therapeutic RNAi, wherein the therapeutic RNAi targets a disease-associated form of the endogenous gene (e.g., a gene encoding a mutant protein, or a gene resulting in abnormal expression of a protein), and the therapeutic mRNA corresponds to a therapeutic form of the endogenous gene (e.g., the second transgene encodes a wild-type or functional form of the mutant protein, or the second transgene results in normal expression of the protein).

In some embodiments of the methods described herein, the disease to be treated is cancer, wherein the cancer is liver cancer, and the pharmaceutical composition comprises an mRNA delivery complex or nanoparticle comprising one or more mRNA encodes one or more proteins involved in liver cancer development and/or progression, wherein the proteins corresponds to one or more genes involved in liver cancer development and/or progression. In some embodiments, the complex or nanoparticle comprises one or more RNAi targets one or more genes involved in liver cancer development and/or progression. In some embodiments, the liver cancer is hepatocellular carcinoma, cholangiocarcinoma, angiosarcoma of the liver, or hemangiosarcoma of the liver. In some embodiments, the one or more genes encoding proteins involved in liver cancer development and/or progression include, but are not limited to, CCND2, RAD23B, GRP78, CEP164, MDM2, and ALDH2, including mutants thereof.

In some embodiments, according to any of the methods described herein, the cancer is hepatocellular carcinoma (HCC). In some embodiments, the HCC is early stage HCC, non-metastatic HCC, primary HCC, advanced HCC, locally advanced HCC, metastatic HCC, HCC in remission, or recurrent HCC. In some embodiments, the HCC is localized resectable (i.e., tumors that are confined to a portion of the liver that allows for complete surgical removal), localized unresectable (i.e., the localized tumors may be unresectable because crucial blood vessel structures are involved or because the liver is impaired), or unresectable (i.e., the tumors involve all lobes of the liver and/or has spread to involve other organs (e.g., lung, lymph nodes, bone). In some embodiments, the HCC is, according to TNM classifications, a stage I tumor (single tumor without vascular invasion), a stage 1 tumor (single tumor with vascular invasion, or multiple tumors, none greater than 5 cm), a stage III tumor (multiple tumors, any greater than 5 cm, or tumors involving major branch of portal or hepatic veins), a stage IV tumor (tumors with direct invasion of adjacent organs other than the gallbladder, or perforation of visceral peritoneum), N1 tumor (regional lymph node metastasis), or M1 tumor (distant metastasis). In some embodiments, the HCC is, according to AJCC (American Joint Commission on Cancer) staging criteria, stage T1, T2, T3, or T4 HCC. In some embodiments, the HCC is any one of liver cell carcinomas, fibrolamellar variants of HCC, and mixed hepatocellular cholangiocarcinomas. In some embodiments, the individual may be a human who has a gene, genetic mutation, or polymorphism associated with hepatocellular carcinoma (e.g., mutation or polymorphism in CCND2, RAD23B, GRP78, CEP164, MDM2, and/or ALDH2) or has one or more extra copies of a gene associated with hepatocellular carcinoma.

In some embodiments of the methods described herein, the disease to be treated is cancer, wherein the cancer is lung cancer, and the pharmaceutical composition comprises an mRNA delivery complex or nanoparticle comprising one or more mRNA encodes one or more proteins involved in lung cancer development and/or progression, wherein the proteins corresponds to one or more genes involved in lung cancer development and/or progression. In some embodiments, the complex or nanoparticle comprises one or more RNAi targets one or more genes involved in lung cancer development and/or progression. In some embodiments, the one or more genes encoding proteins involved in lung cancer development and/or progression include, but are not limited to, SASH1, LATS1, IGF2R, PARK2, KRAS, PTEN, Kras2, Krag, Pas1, ERCC1, XPD, IL8RA, EGFR, Ot₁-AD, EPHX, MMP1, MMP2, MMP3, MMP12, IL1 β, RAS, and AKT, including mutants thereof.

In some embodiments, according to any of the methods described herein, the cancer is lung cancer. In some embodiments, the lung cancer is a non-small cell lung cancer (NSCLC). Examples of NSCLC include, but are not limited to, large-cell carcinoma (e.g., large-cell neuroendocrine carcinoma, combined large-cell neuroendocrine carcinoma, basaloid carcinoma, lymphoepithelioma-like carcinoma, clear cell carcinoma, and large-cell carcinoma with rhabdoid phenotype), adenocarcinoma (e.g., acinar, papillary (e.g., bronchioloalveolar carcinoma, nonmucinous, mucinous, mixed mucinous and nonmucinous and indeterminate cell type), solid adenocarcinoma with mucin, adenocarcinoma with mixed subtypes, well-differentiated fetal adenocarcinoma, mucinous (colloid) adenocarcinoma, mucinous cystadenocarcinoma, signet ring adenocarcinoma, and clear cell adenocarcinoma), neuroendocrine lung tumors, and squamous cell carcinoma (e.g., papillary, clear cell, small cell, and basaloid). In some embodiments, the NSCLC is, according to TNM classifications, a stage T tumor (primary tumor), a stage N tumor (regional lymph nodes), or a stage M tumor (distant metastasis). In some embodiments, the lung cancer is a carcinoid (typical or atypical), adenosquamous carcinoma, cylindroma, or carcinoma of the salivary gland (e.g., adenoid cystic carcinoma or mucoepidermoid carcinoma). In some embodiments, the lung cancer is a carcinoma with pleomorphic, sarcomatoid, or sarcomatous elements (e.g., carcinomas with spindle and/or giant cells, spindle cell carcinoma, giant cell carcinoma, carcinosarcoma, or pulmonary blastoma). In some embodiments, the cancer is small cell lung cancer (SCLC; also called oat cell carcinoma). The small cell lung cancer may be limited-stage, extensive stage or recurrent small cell lung cancer. In some embodiments, the individual may be a human who has a gene, genetic mutation, or polymorphism suspected or shown to be associated with lung cancer (e.g., mutation or polymorphism in SASH1, LATS1, IGF2R, PARK2, KRAS, PTEN, Kras2, Krag, Pas1, ERCC1, XPD, IL8RA, EGFR, Ot₁-AD, EPHX, MMP1, MMP2, MMP3, MMP12, IL1 β, RAS, and/or AKT) or has one or more extra copies of a gene associated with lung cancer.

In some embodiments of the methods described herein, the disease to be treated is cancer, wherein the cancer is renal cell carcinoma (RCC), and the pharmaceutical composition comprises an mRNA delivery complex or nanoparticle comprising one or more mRNA encodes proteins involved in RCC development and/or progression, wherein the proteins corresponds to one or more genes involved in RCC development and/or progression. In some embodiments, the complex or nanoparticle comprises one or more RNAi targets one or more genes involved in RCC development and/or progression. In some embodiments, the one or more genes encoding proteins involved in RCC development and/or progression include, but are not limited to, VHL, TSC1, TSC2, CUL2, MSH2, MLH1, INK4a/ARF, MET, TGF-α, TGF-β1, IGF-I, IGF-IR, AKT, and PTEN, including mutants thereof.

In some embodiments, according to any of the methods described above, the cancer is renal cell carcinoma. In some embodiments, the renal cell carcinoma is an adenocarcinoma. In some embodiments, the renal cell carcinoma is a clear cell renal cell carcinoma, papillary renal cell carcinoma (also called chromophilic renal cell carcinoma), chromophobe renal cell carcinoma, collecting duct renal cell carcinoma, granular renal cell carcinoma, mixed granular renal cell carcinoma, renal angiomyolipomas, or spindle renal cell carcinoma. In some embodiments, the individual may be a human who has a gene, genetic mutation, or polymorphism associated with renal cell carcinoma (e.g., mutation or polymorphism in VHL, TSC1, TSC2, CUL2, MSH2, MLH1, INK4a/ARF, MET, TGF-α, TGF-β1, IGF-I, IGF-IR, AKT, and/or PTEN) or has one or more extra copies of a gene associated with renal cell carcinoma. In some embodiments, the renal cell carcinoma is associated with (1) von Hippel-Lindau (VHL) syndrome, (2) hereditary papillary renal carcinoma (HPRC), (3) familial renal oncocytoma (FRO) associated with Birt-Hogg-Dube syndrome (BHDS), or (4) hereditary renal carcinoma (HRC). In some embodiments, the renal cell carcinoma is at any of stage I, II, III, or IV, according to the American Joint Committee on Cancer (AJCC) staging groups. In some embodiments, the renal cell carcinoma is stage IV renal cell carcinoma.

In some embodiments of the methods described herein, the disease to be treated is cancer, wherein the cancer is a central nervous system (CNS) tumor, and the pharmaceutical composition comprises an mRNA delivery complex or nanoparticle comprising one or more mRNA encodes proteins involved in the CNS tumor development and/or progression, wherein the proteins corresponds to one or more genes involved in CNS tumor development and/or progression. In some embodiments, the complex or nanoparticle comprises one or more RNAi targets one or more genes involved in CNS tumor development and/or progression. In some embodiments, the pharmaceutical composition is administered during or after (such as immediately following) a surgical procedure on the CNS tumor. In some embodiments, the surgical procedure is resection of the CNS tumor. In some embodiments, the pharmaceutical composition is administered into a surgical cavity resulting from the surgical procedure. In some embodiments, the one or more genes encoding proteins involved in the CNS tumor development and/or progression include, but are not limited to, NF1, NF2, SMARCB1, pVHL, TSC1, TSC2, p53, CHK2, MLH1, PMS2, PTCH, SUFU, and XRCC7, including mutants thereof.

In some embodiments, according to any of the methods described herein, the cancer is a CNS tumor. In some embodiments, the CNS tumor is a glioma (e.g., brainstem glioma and mixed gliomas), glioblastoma (also known as glioblastoma multiforme), astrocytoma (such as high-grade astrocytoma), pediatric glioma or glioblastoma (such as pediatric high-grade glioma (HGG) and diffuse intrinsic pontine glioma (DIPG)), CNS lymphoma, germinoma, medulloblastoma, Schwannoma craniopharyogioma, ependymoma, pinealoma, hemangioblastoma, acoustic neuroma, oligodendroglioma, menangioma, neuroblastoma, retinoblastoma, or brain metastasis. In some embodiments, the individual may be a human who has a gene, genetic mutation, or polymorphism suspected or shown to be associated with the CNS tumor (e.g., mutation or polymorphism in NF1, NF2, SMARCB1, pVHL, TSC1, TSC2, p53, CHK2, MLH1, PMS2, PTCH, SUFU, and XRCC7) or has one or more extra copies of a gene associated with the CNS tumor.

In some embodiments of the methods described herein, the disease to be treated is a hematologic disease, and the pharmaceutical composition comprises an mRNA delivery complex or nanoparticle comprising one or more mRNA encoding proteins involved in hematologic disease development and/or progression, wherein the proteins corresponds to one or more genes involved in hematologic disease development and/or progression. In some embodiments, the complex or nanoparticle comprises one or more RNAi targets one or more genes involved in hematologic disease development and/or progression. In some embodiments, the hematologic disease is a hemoglobinopathy, such as sickle-cell disease, thalassemia, or methemoglobinemia, an anemia, such as megaloblastic anemia, hemolytic anemia (e.g., hereditary spherocytosis, hereditary elliptocytosis, congenital dyserythropoietic anemia, glucose-6-phosphate dehydrogenase deficiency, pyruvate kinase deficiency, immune mediated hemolytic anemia, autoimmune hemolytic anemia, warm antibody autoimmune hemolytic anemia, systemic lupus erythematosus, Evans' syndrome, cold autoimmune hemolytic anemia, cold agglutinin disease, paroxysmal cold hemoglobinuria, infectious mononucleosis, alloimmune hemolytic anemia, hemolytic disease of the newborn, or paroxysmal nocturnal hemoglobinuria), aplastic anemia (e.g., Fanconi anemia, Diamond-Blackfan anemia, or acquired pure red cell aplasia), myelodysplastic syndrome, myelofibrosis, neutropenia, agranulocytosis. Glanzmann's thrombasthenia, thrombocytopenia, a myeloproliferative disorder, such as polycethemia vera, erythrocytosis, leukocytosis, or thrombocytosis, or a coagulopathy, such as recurrent thrombosis, disseminated intravascular coagulation, hemophilia (e.g., hemophilia A, hemophilia B, or hemophilia C), Von Willebrand disease, protein S deficiency, antiphospholipid syndrome, or Wiskott-Aldrich syndrome. In some embodiments, the one or more genes encoding proteins involved in hematologic disease development and/or progression include, but are not limited to, HBA1, HBA2, HBB, PROC, ALAS2, ABCB7, SLC25A38, MTTP, FANCA, FANCB, FANCC, FANCD1 (BRCA2), FANCD2, FANCE, FANCF, FANCG, FANCI, FANCJ (BRIP1), FANCL, FANCM, FANCN (PALB2), FANCP (SLX4), FANCS (BRCA1), RAD51C, XPF, ANK1, SPTB, SPTA, SLC4A1, EPB42, and TPI1, including mutants thereof.

In some embodiments of the methods described herein, the disease to be treated is an organ-based disease, and the pharmaceutical composition comprises an mRNA delivery complex or nanoparticle comprising one or more mRNA encoding proteins involved in the organ-based disease development and/or progression, wherein the proteins corresponds to one or more genes involved in organ-based disease development and/or progression. In some embodiments, the complex or nanoparticle comprises one or more RNAi targets one or more genes involved in organ-based disease development and/or progression. In some embodiments, the organ-based disease is a disease of the eye, liver, lung, kidney, heart, nervous system, muscle, or skin. In some embodiments, the disease is a cardiovascular disease, such as coronary heart disease, hypertension, atrial fibrillation, peripheral arterial disease, Marfan syndrome, long QT syndrome, or a congenital heart defect. In some embodiments, the disease is a digestive disease, such as irritable bowel syndrome, ulcerative colitis, Crohn's disease, celiac disease, peptic ulcer disease, gastroesophageal reflux disease, or chronic pancreatitis. In some embodiments, the disease is a urologic disease, such as chronic prostatitis, benign prostatic hyperplasia, or interstitial cystitis. In some embodiments, the disease is a musculoskeletal disease, such as osteoarthritis, osteoporosis, osteogenesis imperfecta, or Paget's disease of bone. In some embodiments, the disease is a skin disease, such as eczema, psoriasis, acne, rosacea, or dermatitis. In some embodiments, the disease is a dental or craniofacial disorder, such as periodontal disease or temporomandibular joint and muscle disorder (TMJD).

In some embodiments of the methods described herein, the disease to be treated is an ocular disease, and the pharmaceutical composition comprises an mRNA delivery complex or nanoparticle comprising one or more mRNA encoding proteins in^{v}olved in ocular disease development and/or progression, wherein the proteins corresponds to one or more genes involved in ocular disease development and/or progression. In some embodiments, the complex or nanoparticle comprises one or more RNAi targets one or more genes involved in ocular disease development and/or progression. In some embodiments, the ocular disease is age-related macular degeneration or the like, retinopathy of prematurity, polypoidal choroidal vasculopathy, diabetic retinopathy, diabetic macular edema, ischemic proliferative retinopathy, retinitis pigmentosa, cone dystrophy, proliferative vitreoretinopathy, retinal artery occlusion, retinal vein occlusion, keratitis, conjunctivitis, uveitis, Leber's disease, retinal detachment, retinal pigment epithelial detachment, neovascular glaucoma, corneal neovascularization, retinochoroidal neovascularization, or inherited retinal disease (such as RPE65-mediated IRD, choroideremia, rhodopsin-linked autosomal dominant retinitis pigmentosa (RHO-adRP), Leber hereditary optic neuropathy (LHON), or Leber congenital amaurosis). In some embodiments, the one or more genes encoding proteins involved in occular disease development and/or progression include, but are not limited to, Rho, PDE6β, ABCA4, RPE65, LRAT, RDS/Peripherin, MERTK, CNGA1, RPGR, IMPDH1, ChR2, GUCY2D, RDS/Peripherin, AIPL1, ABCA4, RPGRIP1, IMPDH1, AIPL1, GUCY2D, LRAT, MERTK, RPGRIP1, RPE65, CEP290, ABCA4, DFNB31, MYO7A, USH1C, CDH23, PCDH15, USH1G, CLRN1, GNAT2, CNGA3, CNGB3, Rs1, OA1, MT-ND4, (OCA1), tyrosinase, p21 WAF-1/OCip1, REP-1, PDGF, Endostatin, Angiostatin, VEGF inhibitor, Opsin, OPNILW, arylsulfatase B, and β-glucuronidase, including mutants thereof.

In some embodiments of the methods described herein, the disease to be treated is a liver disease, and the pharmaceutical composition comprises an mRNA delivery complex or nanoparticle comprising one or more mRNA that encodes proteins involved in liver disease development and/or progression, wherein the proteins corresponds to one or more genes involved in liver disease development and/or progression. In some embodiments, the complex or nanoparticle comprises one or more RNAi targets one or more genes involved in liver disease development and/or progression. In some embodiments, the liver disease is hepatitis, fatty liver disease (alcoholic and nonalcoholic), hemochromatosis, Wilson's disease, progressive familial intrahepatic cholestasis type 3, hereditary fructose intolerance, glycogen storage disease type IV, tyrosinemia type I, argininosuccinate lyase deficiency, citrin deficiency (CTLN2, NICCD), cholesteryl ester storage disease, cystic fibrosis, Alstrom syndrome, congenital hepatic fibrosis, alpha 1-antitrypsin deficiency, glycogen storage disease type II, transthyretin-related hereditary armloidosis, Gilbert's syndrome, cirrhosis, primary biliary cirrhosis, primary sclerosing cholangitis, hemophilia (such as hemophilia A or hemophilia B), or methylmalonic acidemia (MMA). In some embodiments, the one or more genes encoding proteins involved in liver disease development and/or progression include, but are not limited to, ATP7B, ABCB4, ALDOB, GBE1, FAH, ASL, SLC25A13, LIPA, CFTR, ALMS1, HFE, HFE2, HFDE2B, HFE3, SLC11A3/SLC40A, ceruloplasmin, transferrin, A1AT, BCS1L, B3GAT1, B3GAT2, B3GAT3, UGT1A1, UGT1A3, UGT1A4. UGT1A5, UGT1A6, UGT1A7, UGT1A8, UGT1A9, UGTA10, UGT2A1, UGT2A2, UGT2A3, UGT2B4, UGT2B7, GT2B10, UGT2B11, UGT2B15, UGT2B17, UGT2B28, Factor IX, Factor VIII, and MUT (CoA mutase), including mutants thereof.

In some embodiments of the methods described herein, the disease to be treated is a lung disease, and the pharmaceutical composition comprises an mRNA delivery complex or nanoparticle comprising one or more mRNA that encodes proteins involved in lung disease development and/or progression, wherein the proteins corresponds to one or more genes involved in lung cancer development and/or progression. In some embodiments, the complex or nanoparticle comprises one or more RNAi targets one or more genes involved in lung disease development and/or progression. In some embodiments, the lung disease is chronic obstructive pulmonary disease (COPD), asthma, cystic fibrosis, primary ciliary dyskinesia, pulmonary fibrosis, Birt Hogg Dube syndrome, tuberous sclerosis, Kartagener syndrome, α₁-antitrypsin deficiency, pulmonary capillary hemangiomatosis (PCH), or hereditary heamorrhagic telangiectasia. In some embodiments, the one or more genes encoding proteins involved in lung disease development and/or progression include, but are not limited to, EIF2AK4, IREB2, HHIP, FAM13A, IL1RL1, TSLP, IL33, IL25, CFTR, DNAI1, DNAH5, TXNDC3, DNAH11, DNAI2, KTU, RSPH4A, RSPH9, LRRC50, TERC, TERT, SFTPC, SFTPA2, FLCN, TSC1, TSC2, A1AT, ENG, ACVRL1, and MADH4, including mutants thereof.

In some embodiments of the methods described herein, the disease to be treated is a kidney disease, and the pharmaceutical composition comprises an mRNA delivery complex or nanoparticle comprising one or more mRNA that encodes proteins involved in kidney disease development and/or progression, wherein the proteins corresponds to one or more genes involved in kidney disease development and/or progression. In some embodiments, the complex or nanoparticle comprises one or more RNAi targets one or more genes involved in kidney disease development and/or progression. In some embodiments, the kidney disease is cystic kidney disease (e.g., autosomal dominant polycystic kidney disease, autosomal recessive polycystic kidney disease, nephronophthisis, or medullary sponge kidney), Alport's syndrome, Banter's syndrome, congenital nephrotic syndrome, nail-patella syndrome, primary immune glomerulonephritis, reflux nephropathy, or haemolytic uraemic syndrome. In some embodiments, the one or more genes encoding proteins involved in kidney disease development and/or progression include, but are not limited to, PKD1, PKD2, PKD3, fibrocystin, NPHP1, NPHP2, NPHP3, NPHP4, NPHP5, NPHP6. NPHP7. NPHP8, NPHP9, NPHP11, NPHP11, NPHPL1, GDNF, COL4A5, COL4A3, COL4A4, SLC12A2 (NKCC2), ROMK/KCNJ1, CLCNKB, BSND, CASR, SLC12A3 (NCCT), and ADAMTS13, including mutants thereof.

In some embodiments of the methods described herein, the disease to be treated is a muscle disease, and the pharmaceutical composition comprises an mRNA delivery complex or nanoparticle comprising one or more mRNA that encodes proteins involved in muscle disease development and/or progression, wherein the proteins corresponds to one or more genes involved in muscle disease development and/or progression. In some embodiments, the complex or nanoparticle comprises one or more RNAi targets one or more genes involved in muscle disease development and/or progression. In some embodiments, the muscle disease is myopathy (e.g., mitochondrial myopathy), muscular dystrophy (e.g., Duchenne, Becker, Emery-Dreifuss, facioscapulohumeral, myotonic, congenital, distal, limb-girdle, and oculopharyngeal), cerebral palsy, fibrodysplasia ossificans progressiva, dermatomyositis, compartment syndrome, myasthenia gravis, amyotrophic lateral sclerosis, rhabdomyolysis, polymyositis, fibromyalgia, myotonia, myofascial pain syndrome. In some embodiments, the one or more genes encoding proteins involved in muscle disease development and/or progression include, but are not limited to, DMD, LAMA2, collagen VI (COL6A1, COL6A2, or COL6A3), POMT1, POMT2, FKTN, FKRP, LARGE1, POMGNT1, ISPD, SEPN1, LMNA, DYSF, EMD, DUX4, DMPK, ZNF9, PABPN1, CAV3, CAPN3, SGCA, SGCB, SGCG, SGCD, TTN, ANO5, DNAJB6, HNRNPDL, MYOT, TCAP, TNPO3, TRAPPC11, and TRIM32, including mutants thereof.

In some embodiments of the methods described herein, the disease to be treated is a nervous system disease (such as a central nervous system disease), and the pharmaceutical composition comprises an mRNA delivery complex or nanoparticle comprising one or more mRNA encoding proteins involved in nervous system disease development and/or progression, wherein the proteins corresponds to one or more genes involved in nervous system development and/or progression. In some embodiments, the complex or nanoparticle comprises one or more RNAi targets one or more genes involved in nervous system development and/or progression. In some embodiments, the nervous system disease is adrenoleukodystrophy, Angelman syndrome, ataxia telangiectasia, Charcot-Marie-Tooth syndrome, Cockayne syndrome, essential tremor, fragile X syndrome, Friedreich's ataxia, Gaucher disease, Lesch-Nyhan syndrome, maple syrup urine disease, Menkes syndrome, narcolepsy, neurofibromatosis, Niemann-Pick disease, phenylketonuria, Prader-Willi syndrome, Refsum disease, Rett syndrome, spinal muscular atrophy, spinocerebellar ataxia. Tangier disease, Tay-Sachs disease, tuberous sclerosis, Von Hippel-Lindau syndrome, Williams syndrome, Wilson's disease, Zellweger syndrome, attention deficit/hyperactivity disorder (ADHD), autism, bipolar disorder, depression, epilepsy, migraine, multiple sclerosis, myelopathy, Alzheimer's, Huntington's, Parkinson's, Tourette's, CLN2 disease (such as CLN2 disease caused by TPP1 deficiency), or mucopolysaccharidosis (such as mucopolysaccharidosis type I (MPS I) or mucopolysaccharidosis type II (MPS II)). In some embodiments, the one or more genes encoding proteins involved in nervous system disease development and/or progression include, but are not limited to, ALD, PS1 (AD3), PS2 (AD4), SOD1, UBE3A, ATM, PMP22, MPZ, LITAF, EGR2, MFN2, KIF1B, RAB7A, LMNA, TRPV4, BSCL2, GARS, NEFL, HSPB1, GDAP1, HSPB8, MTMR2, SBF2, SH3TC2, NDRG1, PRX, FGD4, FIG4, DNM2, YARS, GJB1, PRPS1, CSA, CSB, Cx26, EPM2A, ETM1 (FET1), ETM2, FMR1, frataxin, GBA, HTT, HPRT1, BCKDH, ATP7A, ATP7B, HLA-DQB1, HLA-DQA1, HLA-DRB1, NF1, NF2, SMPD1, NPC1, NPC2, LRRK2, PARK7, PINK1, PRKN, SNCA, UCHL1, PAH, PHYH, PEX7, MeCP2, SMN1, ATXN genes (e.g., ATXN1, ATXN2, etc), ABC1, HEXA, TSC1, TSC2, VHL, CLIP2, ELN, GTF21, GTF2I, GRF2IRD1, LIMK1, PXR1, TPP1, IDUA, and IDS, including mutants thereof.

In some embodiments of the methods described herein, the disease to be treated is cancer, wherein the cancer is a hematological malignancy, and the pharmaceutical composition comprises an mRNA delivery complex or nanoparticle comprising one or more mRNA that encodes proteins involved in hematological malignancy development and/or progression, wherein the proteins corresponds to one or more genes involved in hematological malignancy development and/or progression. In some embodiments, the complex or nanoparticle comprises one or more RNAi targets one or more genes involved in hematological malignancy development and/or progression. In some embodiments, the one or more genes encoding proteins involved in hematological malignancy development and/or progression include, but are not limited to, GLI1, CTNNB1, eIF5A, mutant DDX3X, Hexokinase II, histone methyltransferase EZH2, ARK5, ALK, MUC1, HMGA2, IRF1, RPN13, HDAC11, Rad51, Spry2, mir-146a, mir-146b, survivin, MDM2, MCL1, CMYC, XBP1 (spliced and unspliced), SLAMF7, CS1, Erbb4, Cxcr4 (waldenstroms macroglobulinemia), Myc, Bcl2, Prdx1 and Prdx2 (burkitts lymphoma), Bcl6, Idh1, Idh2, Smad, Ccnd2, Cyclin d1-2, B7-h1 (pdl-1), and Pyk2, including mutants thereof.

In some embodiments of the methods described herein, the disease to be treated is a hematological malignancy including, without limitation, leukemias, including acute leukemias (such as acute lymphocytic leukemia, acute myelocytic leukemia, acute myeloid leukemia and myeloblastic, promyelocytic, myelomonocytic, monocytic and erythroleukemia), chronic leukemias (such as chronic myelocytic (granulocytic) leukemia, chronic myeloid leukemia, and chronic lymphocytic leukemia), polycythemia vera, B cell lymphoma (such as splenic marginal zone lymphoma, extranodal marginal zone B cell lymphoma, nodal marginal zone B cell lymphoma, follicular lymphoma, primary cutaneous follicle center lymphoma, mantle cell lymphoma, diffuse large B cell lymphoma, lymphomatoid granulomatosis, primary mediastinal large B cell lymphoma, intravascular large B cell lymphoma, ALK+ large B cell lymphoma, plasmablastic lymphoma, primary effusion lymphoma, and Burkitt lymphoma), T cell and/or NK cell lymphoma (such as adult T cell lymphoma, extranodal NK-T cell lymphoma, enteropathy-associated T cell lymphoma, hepatosplenic T cell lymphoma, blastic NK cell lymphoma, primary cutaneous anaplastic large cell lymphoma, lymphomatoid papulosis, peripheral T cell lymphoma, angioimmunoblastic T cell lymphoma, and anaplastic large cell lymphoma), Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia.

In some embodiments of the methods described herein, the disease to be treated is a viral infectious disease, and the pharmaceutical composition comprises an mRNA delivery complex or nanoparticle comprising one or more mRNA that encodes proteins involved in the viral infectious disease development and/or progression, wherein the proteins corresponds to one or more genes involved in viral infectious disease development and/or progression. In some embodiments, the complex or nanoparticle comprises one or more RNAi targets one or more genes involved in viral infectious disease development and/or progression. In some embodiments, the viral infectious disease is characterized by infection with hepatitis mRNA, human immunodeficiency mRNA (HIV), picornamRNA, poliomRNA, enteromRNA, human Coxsackie mRNA, influenzan mRNA, rhinomRNA, echomRNA, rubellan mRNA, encephalitis mRNA, rabies mRNA, herpes mRNA, papillomamRNA, polyoman mRNA, RSV, adenomRNA, yellow fever mRNA, dengue mRNA, parainfluenzan mRNA, hemorrhagic mRNA, pox mRNA, varicella zoster mRNA, parainfluenzan mRNA, reomRNA, orbimRNA, rotamRNA, parvomRNA, African swine fever mRNA, measles, mumps or Norwalk mRNA. In some embodiments, the viral infectious disease is characterized by infection with an oncogenic mRNA including, but not limited to, CMV, EBV, HBV, KSHV, HPV, MCV, HTLV-1, HIV-1, and HCV In some embodiments, the one or more genes encoding proteins involved in the viral infectious disease development and/or progression include, but are not limited to, genes encoding RSV nucleocapsid, Pre-gen/Pre-C, Pre-S1, Pre-S2/S,X, HBV conserved sequences, HIV Gag polyprotein (p55), HIV Pol polyprotein, HIV Gag-Pol precursor (p160), HIV matrix protein (MA, p17), HIV capsid protein (CA, p24), HIV spacer peptide 1 (SP1, p2), HIV nucleocapsid protein (NC, p9), HIV spacer peptide 2 (SP2, p1), HIV P6 protein, HIV reverse transcriptase (RT, p50), HIV RNase H (p15), HIV integrase (IN, p31), HIV protease (PR, p10), HIV Env (gp160), gp120, gp41, HIV transactivator (Tat), HIV regulator of expression of virion proteins (Rev), HIV lentimRNA protein R (Vpr), HIV Vif, HIV negative factor (Nef), HIV mRNA protein U (Vpu), human CCR5, miR-122, EBOV polymerase L, VP24, VP40, GP/sGP, VP30, VP35, NPC1, and TIM-1, including mutants thereof.

In some embodiments of the methods described herein, the disease or condition to be treated is an autoimmune or inflammatory disease or condition, and the pharmaceutical composition comprises an mRNA delivery complex or nanoparticle comprising one or more mRNA that encodes proteins involved in the autoimmune or inflammatory disease or condition development and/or progression, wherein the proteins corresponds to one or more genes involved in the autoimmune or inflammatory disease or condition development and/or progression. In some embodiments, the complex or nanoparticle comprises one or more RNAi targets one or more genes involved in autoimmune or inflammatory disease or condition development and/or progression. In some embodiments, the autoimmune or inflammatory disease or condition is acne, allergies, anaphylaxis, asthma, celiac disease, diverticulitis, glomerulonephritis, inflammatory bowel disease, interstitial cystitis, lupus, otitis, pelvic inflammatory disease, rheumatoid arthritis, sarcoidosis, or vasculitis. In some embodiments, the one or more genes encoding proteins involved in the autoimmune or inflammatory disease or condition development and/or progression include, but are not limited to, genes encoding molecules of the complement system (CD46, CD59, CFB, CFD, CFH, CFHR1, CFHR2, CFHR3, CFHR4, CFHR5, CF1, CFP, CR1, CR1L, CR2, CQA, C1QB, C1QC, C1R, C1S, C2, C3, C3AR1, C4A, C4B, C5, C5AR1, C6, C7, C8A, C8B, C8G, C9, ITGAM, ITGAX, and ITGB2), including mutants thereof.

In some embodiments of the methods described herein, the disease or condition to be treated is a lysosomal storage disease, and the pharmaceutical composition comprises an mRNA delivery complex or nanoparticle comprising one or more mRNA that encodes proteins involved in the lysosomal storage disease development and/or progression, wherein the proteins corresponds to one or more genes involved in lysosomal storage disease development and/or progression. In some embodiments, the complex or nanoparticle comprises one or more RNAi targets one or more genes involved in lysosomal storage disease development and/or progression. In some embodiments, the lysosomal storage disease is Sphingolipidoses (e.g., Farber disease, Krabbe disease (infantile onset, late onset), galactosialidosis, gangliosidoses (e.g., Fabrv disease, Schindler disease, GM1 gangliosidosis (Infantile. Juvenile, Adult/Chronic), GM2 gangliosidosis (e.g., Sandhoff disease (Infantile, Juvenile, Adult onset), Tay-Sachs)), Gaucher Disease (Type I, Type II, Type III), Lysosomal acid lipase deficiency (Early onset, Late onset), Niemann-Pick disease (Type A. Type B), Sulfatidosis (e.g., Metachromatic Leukodystrophy (MLD), Multiple sulfatase deficiency)), Mucopolysaccharidoses (e.g., MPS I Hurler Syndrome, MPS I S Scheie Syndrome, MPS I H-S Hurler-Scheie Syndrome. Type II (Hunter syndrome), Type III (Sanfilippo syndrome), Type IV (Morquio), Type VI (Maroteaux-Lamy syndrome), Type VII (Sly Syndrome), Type IX (Hyaluronidase deficiency)), Mucolipidosis (e.g., Type I (Sialidosis), Type II (I-cell disease), Type III (Pseudo-Hurler Polydystrophy/Phosphotransferase deficiency), Type IV (Mucolipidin 1 deficiency)), Lipidoses (e.g., Niemann-Pick disease (Type C, Type D), Neuronal Ceroid Lipofuscinoses, Wolman disease), Alpha-mannosidosis, Beta-mannosidosis, Aspartylglucosaminuria, Fucosidosis, Lysosomal Transport Diseases (e.g., Cystinosis, Pycnodysostosis, Salla disease/Sialic Acid Storage Disease, Infantile Free Sialic Acid Storage Disease (ISSD)). Cholesteryl ester storage disease. In some embodiments, the one or more genes encoding proteins involved in the lysosomal storage disease development and/or progression include, but are not limited to, genes encoding ceramidase, Alpha-galactosidase (A, B), Beta-galactosidase, Hexosaminidase A, Sphingomyelinase, Lysosomal acid lipase, Saposin B, sulfatase. Hyaluronidase, Phosphotransferase, Mucolipidin 1, aspartylglucosaminidase, alpha-D-mannosidase, beta-mannosidase, alpha-L-fucosidase, cystinosin, cathepsin K, sialin, SLC17A5, acid alpha-glucosidase, LAMP2, including mutants thereof.

In some embodiments of the methods described herein, the disease or condition to be treated is a glycogen storage disease, and the pharmaceutical composition comprises an mRNA delivery complex or nanoparticle comprising one or more mRNA that encodes proteins involved in the glycogen storage disease development and/or progression, wherein the proteins corresponds to one or more genes involved in glycogen storage disease development and/or progression. In some embodiments, the complex or nanoparticle comprises one or more RNAi targets one or more genes involved in glycogen storage disease development and/or progression. In some embodiments, the glycogen storage disease is von Gierke's disease, Pompe's disease, Cori's disease or Forbes' disease, Andersen disease, McArdle disease, Hers' disease, Tarui's disease, Fanconi-Bickel syndrome, or Red cell aldolase deficiency. In some embodiments, the one or more genes encoding proteins involved in the glycogen storage disease development and/or progression include, but are not limited to, genes encoding glycogen synthase, glucose-6-phosphatase, acid alpha-glucosidase, glycogen debranching enzyme, glycogen branching enzyme, muscle glycogen phosphorylase, liver glycogen phosphorylase, muscle phosphofructokinase. Phosphorylase kinase, glucose transporter, GLUT2, Aldolase A, and β-enolase, including mutants thereof.

In some embodiments of the methods described herein, the condition to be treated is characterized by abnormal cholesterol levels (such as abnormally high LDL levels, e.g., LDL above about 100 mg/dL, and/or abnormally low HDL levels, e.g., HDL below about 40-50 mg/dL), including, e.g., familial hypercholesterolemia (such as homozygous familial hypercholesterolemia (HoFH)), and the pharmaceutical composition comprises an mRNA delivery complex or nanoparticle comprising one or more mRNA that encodes proteins involved in cholesterol transport and/or metabolism, wherein the proteins corresponds to one or more genes involved in cholesterol transport and/or metabolism. In some embodiments, the complex or nanoparticle comprises one or more RNAi targets one or more genes involved in cholesterol transport and/or metabolism. In some embodiments, the one or more genes encoding proteins involved in cholesterol transport and/or metabolism include, but are not limited to, low-density lipoprotein (LDL) receptor (LDLR), apolipoprotein B (ApoB), low-density lipoprotein receptor adapter protein 1 (LDLRAP1), and PCSK9, including mutants thereof.

In some embodiments, an mRNA delivery complex or nanoparticle as described herein is used to activate or increase LDLR expression.

In some embodiments, an mRNA delivery complex or nanoparticle as described herein is used to activate or increase ApoB expression.

In some embodiments, an mRNA delivery complex or nanoparticle as described herein is used to activate or increase LDLRAP1 expression.

In some embodiments, an mRNA delivery complex or nanoparticle as described herein further comprises a RNAi (such as siRNA), wherein the RNAi repress PCSK9 expression.

In some embodiments of the methods described herein, the disease to be treated is a genetic disease, such as a hereditary disease, and the pharmaceutical composition comprises an mRNA delivery complex or nanoparticle comprising one or more mRNA that encodes proteins involved in the genetic disease development and/or progression, wherein the proteins corresponds to one or more genes involved in the genetic disease development and/or progression. In some embodiments, the complex or nanoparticle comprises one or more RNAi targets one or more genes involved in the genetic disease development and/or progression. In some embodiments, the genetic disease includes, but is not limited to, 22q11.2 deletion syndrome, achondroplasia, Alpha-1 Antitrypsin Deficiency. Angelman syndrome, Autosomal dominant polycystic kidney disease, breast cancer, Canavan disease, Charcot-Marie-Tooth disease, cancer, Color blindness, Cystic fibrosis, Duchenne muscular dystrophy, Factor V Leiden thrombophilia, Familial Mediterranean Fever, Fragile X syndrome, Gaucher disease, Haemochromatosis, Haemophilia, Huntington's disease, Marfan syndrome, Myotonic dystrophy. Osteogenesis imperfecta, Parkinson's disease, Phenylketonuria, Polycystic kidney disease, porphyria, Prader-Willi syndrome, progeria, SCID, Sickle-cell disease. Spinal muscular atrophy, Tay-Sachs disease, thalassemia, Trimethylamine, and Wilson's disease. In some embodiments, the genes involved in the genetic disease development and/or progression include, but are not limited to, AAT, ADA, ALAD, ALAS2, APC, ASPM, ATP7B, BDNF, BRCA1, BRCA2, CFTR, COL1A1, COL1A2, COMT, CNBP, CPOX, CREBBP, CRH, CRTAP, CXCR4, DHFR, DMD, DMPK, F5, FBN1, FECHFGFR3, FGR3, FIX, FVIII, FMO3, FMR1, GARS, GBA, HBB, HEXA, HFE, HMBS, HTT, IL2RG, KRT14, KRT5, LMNA, LRRK2, MEFV, MLH1, MSH2, MSH6, PAH, PARK2, PARK3, PARK7, PGL2, PHF8, PINK1, PKD1, PKD2, PMS1, PMS2, PPOX, RHO, SDHB, SDHC, SDHD, SMNI, SNCA, SRY, TSC1, TSC2, UCHL1, UROD, UROS, MEFV, APP, GAST, INS, LCK, LEP, LIF, MCM6, MYH7, MYOD, NPPB, OSM, PKC, PIP, SLC18A2, TBX1, Transthyretin, MDSI-EVI1, PRDM16, SETBP1, ß-Globin, and LPL, including mutants thereof.

In some embodiments of the methods described herein, the disease to be treated is an aging or degenerative disease, and the pharmaceutical composition comprises an mRNA delivery complex or nanoparticle comprising one or more mRNA that encodes proteins involved in the aging or degenerative disease development and/or progression, wherein the proteins corresponds to one or more genes involved in the aging or degenerative disease development and/or progression. In some embodiments, the complex or nanoparticle comprises one or more RNAi targets one or more genes involved in the aging or degenerative disease development and/or progression. In some embodiments, the one or more genes encoding proteins involved in the aging or degenerative disease development and/or progression include, but are not limited to, keratin K6A, keratin K6B, keratin 16, keratin 17, p53, ß-2 adrenergic receptors (ADRB2), TRPV1, VEGF, VEGFR, HIF-1, and caspase-2, including mutants thereof.

In some embodiments of the methods described herein, the disease to be treated is a fibrotic or inflammatory disease, and the pharmaceutical composition comprises an mRNA delivery complex or nanoparticle comprising one or more mRNA that encodes proteins involved in the fibrotic or inflammatory disease development and/or progression, wherein the proteins corresponds to one or more genes involved in the fibrotic or inflammatory disease development and/or progression. In some embodiments, the complex or nanoparticle comprises one or more RNAi targets one or more genes involved in the fibrotic or inflammatory disease development and/or progression. In some embodiments, the one or more genes encoding proteins involved in the fibrotic or inflammatory disease development and/or progression are selected from the group consisting of SPARC, CTGF, TGFβ1, TGFβ receptors 1, TGFβ receptors 2, TGFβ receptors 3, VEGF, Angiotensin II, TIMP, HSP47, thrombospondin, CCN1, LOXL2, MMP2, MMP9, CCL2, Adenosine receptor A2A, Adenosine receptor A2B, Adenylyl cyclase, Smad 3, Smad 4, Smad 7, SOX9, arrestin, PDCD4, PAI-1, NF-κB, and PARP-1, including mutants thereof.

In some embodiments of the methods described herein, the pharmaceutical composition comprises an mRNA delivery complex or nanoparticle comprising one or more mRNA and/or RNAi (such as siRNA) that modulates the expression of one or more miRNAs involved in a disease or condition. In some embodiments, the mRNA delivery complex or nanoparticle comprises the one or more miRNAs, or is to be used in combination with the one or more miRNAs. In some embodiments, the disease or condition includes, but is not limited to, hepatitis B, hepatitis C, polycystic liver and kidney disease, cancer, cardiovascular disease, cardiac failure, cardiac hypertrophy, neurodevelopmental disease, fragile X syndrome, Rett syndrome, Down syndrome, Alzheimer's disease, Huntington's disease, schizophrenia, inflammatory disease, rheumatoid arthritis, systemic lupus erythematosus, psoriasis, and skeletal muscle disease. In some embodiments, the one or more miRNAs include, but are not limited to, has-mir-126*, Has-miR-191, has-mir-205, has-mir-21, hsa-let-7a-2, let-7 family, let-7c, let-7f-1, miR-1, miR-100, miR-103, miR-103-1, miR-106b-25, miR-107, miR-10b, miR-112, miR-122, miR-125b, miR-125b-2, miR125b1, miR-126, miR-128a, mIR-132, miR-133, miR-133b, miR135, miR-140, miR-141, miR-142-3p, miR143, miR-143, miR145, miR-145, miR-146, miR-146b, miR150, miR-155, miR-15a, miR-15b, miR16, miR-16, miR-17-19 family, miR-173p, miR17-5p, miR-17-5p, miR-17-92, miR-181a, miR-181b, miR-184, miR-185, miR-189, miR-18a, miR-191, miR-192, miR-193a, miR-193b, miR-194, miR-195, miR-196a, miR-198, miR-199, miR-199a, miR-19a, miR-19b-1, miR200a, miR-200a, miR-200b, miR200c, miR-200c, miR-203, miR-205, miR-208, miR-20a, miR-21, miR-214, miR-221, miR-222, miR-223, miR-224, miR-23, miR-23a, miR-23b, miR-24, miR-26a, miR-26b, miR-27b, miR-29, miR-298, miR-299-3p, miR-29c, miR-30a-5p, miR-30c, miR-30d, miR-30e-5p, miR31, miR-34, miR342, miR-381, miR-382, miR-383, miR-409-3p, miR-45, miR-61, miR-78, miR-802, miR-9, miR-92a-1, miR-99a, miR-let7, miR-let7a, and miR-let7g.

In some embodiments of the methods described herein, the pharmaceutical composition is administered to the individual by any of intravenous, intratumoral, intraarterial, topical, intraocular, ophthalmic, intraportal, intracranial, intracerebral, intracerebroventricular, intrathecal, intravesicular, intradermal, subcutaneous, intramuscular, intranasal, intratracheal, pulmonary, intracavity, or oral administration. In some embodiments, the pharmaceutical composition is administered to the individual via convection enhanced delivery. In some embodiments, the pharmaceutical composition is administered to the individual via an infusion pump. In some embodiments, the pharmaceutical composition is administered to the individual via an osmotic pump. In some embodiments, the pharmaceutical composition is administered to the individual via a catheter, such as a catheter with a needle. In some embodiments, the pharmaceutical composition is administered to the individual via an intracoronary local drug delivery catheter.

In some embodiments of the methods described herein, the individual is a mammal. In some embodiments, the individual is human.

### Methods of Cell Delivery

In some embodiments, there is provided a method of delivering one or more mRNA into a cell comprising contacting the cell with an mRNA delivery complex or nanoparticle as described herein, wherein the complex or nanoparticle comprises the one or more mRNA. In some embodiments, the mRNA is modified (e.g., wherein at least one modified nucleoside is 5-methoxyuridine (5moU)). In some embodiments, the complex or nanoparticle comprises a CPP comprising the amino acid sequence of a PEP-1 peptide, a PEP-2 peptide, a VEPEP-3 peptide, a VEPEP-6 peptide, a VEPEP-9 peptide, or an ADGN-100 peptide. In some embodiments, the contacting of the cell with the complex or nanoparticle is carried out in vivo. In some embodiments, the contacting of the cell with the complex or nanoparticle is carried out ex vivo. In some embodiments, the contacting of the cell with the complex or nanoparticle is carried out in vitro. In some embodiments, the cell is an immortalized cell, such as a cell from a cell line. In some embodiments, the cell is a primary cell, such as a cell from an individual. In some embodiments, the cell is an immune cell, such as a granulocyte, a mast cell, a monocyte, a dendritic cell, a B cell, a T cell, or a natural killer cell. In some embodiments, the cell is a peripheral blood-derived T cell, a central memory T cell, a cord blood-derived T cell, or a hematopoietic stem cell or other precursor cell. In some embodiments, the T cell is an immortalized T cell, such as a T cell from a T cell line. In some embodiments, the T cell is a primary T cell, such as a T cell of an individual. In some embodiments, the cell is a T cell, and the contacting is carried out after activating the T cell. In some embodiments, the cell is a T cell, and the contacting is carried out at least 12 hours (such as at least about any of 12 hours, 1 day, 2 days, 3 days, 4 days, 5 days, 6 days, or more) after activating the T cell. In some embodiments, the T cell is activated using an anti-CD3CD28 reagent (such as microbeads). In some embodiments, the cell is a fibroblast. In some embodiments, the fibroblast is a primary fibroblast, such as a fibroblast of an individual. In some embodiments, the cell is a muscle cell. In some embodiments, the cell is a cardiac cell. In some embodiments, the cell is a hepatocyte. In some embodiments, the hepatocyte is a primary hepatocyte, such as a hepatocyte of an individual. In some embodiments, the cell is a human lung progenitor cell (LPC). In some embodiments, the cell is a neuronal cell. In some embodiments, the mRNA is useful for the treatment of a disease, such as any of the diseases to be treated described herein (e.g., cancer, diabetes, autoimmune diseases, inflammatory diseases, fibrotic diseases, viral infectious diseases, hereditary diseases, ocular diseases, and aging and degenerative diseases). In some embodiments, the complex or nanoparticle further comprises one or more RNAi. In some embodiments, the RNAi is useful for the treatment of the disease.

Thus, in some embodiments, there is provided a method of delivering one or more mRNA into a cell comprising contacting the cell with an mRNA delivery complex or nanoparticle as described herein, wherein the mRNA delivery complex or nanoparticle comprises the one or more mRNA and a CPP comprising the amino acid sequence of a PEP-1 peptide, a PEP-2 peptide, a VEPEP-3 peptide, a VEPEP-6 peptide, a VEPEP-9 peptide, or an ADGN-100 peptide. In some embodiments, the VEPEP-3 peptide comprises the amino acid sequence of any one of SEQ ID NOs: 1-14, 75, and 76. In some embodiments, the VEPEP-6 peptide comprises the amino acid sequence of any one of SEQ ID NOs: 15-40, and 77. In some embodiments, the VEPEP-9 peptide comprises the amino acid sequence of any one of SEQ ID NOs: 41-52, and 78. In some embodiments, the ADGN-100 peptide comprises the amino acid sequence of any one of SEQ ID NOs: 53-70, 79, and 80. In some embodiments, the mRNA encodes a therapeutic protein, for example, a tumor suppressor protein. In some embodiments, the mRNA encodes a CAR. In some embodiments, the complex or nanoparticle comprises a plurality of mRNA encoding a plurality of protein. In some embodiments, the complex or nanoparticle comprises a plurality of mRNA encoding a single protein. In some embodiments, the complex or nanoparticle comprises a single mRNA encoding a first protein and a second protein. In some embodiments, the complex or nanoparticle further comprises a RNAi such as siRNA, such as an RNAi targeting an endogenous gene, e.g., a disease-associated endogenous gene. In some embodiments, the RNAi targets an exogenous gene. In some embodiments, the RNAi is a therapeutic RNAi targeting an endogenous gene involved in a disease or condition, and the protein is a therapeutic protein useful for treating the disease or condition. In some embodiments, the complex or nanoparticle comprises a therapeutic mRNA and a therapeutic RNAi, wherein the therapeutic RNAi targets a disease-associated form of the endogenous gene (e.g., a gene encoding a mutant protein, or a gene resulting in abnormal expression of a protein), and the therapeutic mRNA corresponds to a therapeutic form of the endogenous gene (e.g., the second transgene encodes a wild-type or functional form of the mutant protein, or the second transgene results in normal expression of the protein). In some embodiments, the contacting of the cell with the complex or nanoparticle is carried out in vivo. In some embodiments, the contacting of the cell with the complex or nanoparticle is carried out ex vivo. In some embodiments, the contacting of the cell with the complex or nanoparticle is carried out in vitro. In some embodiments, the cell is an immortalized cell, such as a cell from a cell line. In some embodiments, the cell is a primary cell, such as a cell from an individual. In some embodiments, the cell is an immune cell, such as a granulocyte, a mast cell, a monocyte, a dendritic cell, a B cell, a T cell, or a natural killer cell. In some embodiments, the T cell is an immortalized T cell, such as a T cell from a T cell line. In some embodiments, the T cell is a primary T cell, such as a T cell of an individual. In some embodiments, the cell is a fibroblast. In some embodiments, the fibroblast is a primary fibroblast, such as a fibroblast of an individual. In some embodiments, the cell is a muscle cell. In some embodiments, the cell is a cardiac cell. In some embodiments, the cell is a hepatocyte. In some embodiments, the hepatocyte is a primary hepatocyte, such as a hepatocyte of an individual. In some embodiments, the cell is a human lung progenitor cell (LPC). In some embodiments, the cell is a neuronal cell. In some embodiments, the mRNA is useful for the treatment of a disease, such as any of the diseases to be treated described herein (e.g., cancer, diabetes, autoimmune diseases, inflammatory diseases, fibrotic diseases, viral infectious diseases, hereditary diseases, ocular diseases, and aging and degenerative diseases). In some embodiments, the mRNA is useful for modulating a protein involved in a disease, such as any of the diseases to be treated described herein (e.g., cancer, diabetes, autoimmune diseases, inflammatory diseases, fibrotic diseases, viral infectious diseases, hereditary diseases, ocular diseases, and aging and degenerative diseases). In some embodiments, the cell-penetrating peptide is an ADGN-100 peptide or a AGDN-106 peptide.

In some embodiments, there is provided a method of delivering one or more mRNA into a T cell comprising contacting the cell with an mRNA delivery complex or nanoparticle as described herein, wherein the complex or nanoparticle comprises the one or more mRNA and a CPP selected from the group consisting of PEP-1 peptides, PEP-2 peptides, PEP-3 peptides, VEPEP-3 peptides, VEPEP-6 peptides, VEPEP-9 peptides, and ADGN-100 peptides. In some embodiments, the contacting of the T cell with the complex or nanoparticle is carried out in vivo. In some embodiments, the contacting of the T cell with the complex or nanoparticle is carried out ex vivo. In some embodiments, the contacting of the T cell with the complex or nanoparticle is carried out in vitro. In some embodiments, the T cell is an immortalized T cell, such as a T cell from a T cell line. In some embodiments, the T cell is a primary T cell, such as a T cell of an individual. In some embodiments, the mRNA is useful for the treatment of a disease, such as any of the diseases to be treated described herein. In some embodiments, the complex or nanoparticle further comprises one or more RNAi. In some embodiments, the RNAi is useful for the treatment of the disease.

In some embodiments, there is provided a method of delivering one or more mRNA into a fibroblast comprising contacting the fibroblast with an mRNA delivery complex or nanoparticle as described herein, wherein the complex or nanoparticle comprises the one or more mRNA and a CPP selected from the group consisting of PEP-1 peptides, PEP-2 peptides, PEP-3 peptides, VEPEP-3 peptides, VEPEP-6 peptides, VEPEP-9 peptides, and ADGN-100 peptides. In some embodiments, the contacting of the fibroblast with the complex or nanoparticle is carried out in vivo. In some embodiments, the contacting of the fibroblast with the complex or nanoparticle is carried out ex vivo. In some embodiments, the contacting of the fibroblast with the complex or nanoparticle is carried out in vitro. In some embodiments, the fibroblast is an immortalized fibroblast, such as a fibroblast from a fibroblast line. In some embodiments, the fibroblast is a primary fibroblast, such as a fibroblast of an individual. In some embodiments, the mRNA is useful for the treatment of a disease, such as any of the diseases to be treated described herein. In some embodiments, the complex or nanoparticle further comprises one or more RNAi. In some embodiments, the RNAi is useful for the treatment of the disease.

In some embodiments, there is provided a method of delivering one or more mRNA into a hepatocyte comprising contacting the hepatocyte with an mRNA delivery complex or nanoparticle as described herein, wherein the complex or nanoparticle comprises the one or more mRNA and a CPP selected from the group consisting of PEP-1 peptides, PEP-2 peptides, PEP-3 peptides, VEPEP-3 peptides, VEPEP-6 peptides, VEPEP-9 peptides, and ADGN-100 peptides. In some embodiments, the contacting of the hepatocyte with the complex or nanoparticle is carried out in vivo. In some embodiments, the contacting of the hepatocyte with the complex or nanoparticle is carried out ex vivo. In some embodiments, the contacting of the hepatocyte with the complex or nanoparticle is carried out in vitro. In some embodiments, the hepatocyte is an immortalized hepatocyte, such as a hepatocyte from a hepatocyte line. In some embodiments, the hepatocyte is a primary hepatocyte, such as a hepatocyte of an individual. In some embodiments, the mRNA is useful for the treatment of a disease, such as any of the diseases to be treated described herein. In some embodiments, the complex or nanoparticle further comprises one or more RNAi. In some embodiments, the RNAi is useful for the treatment of the disease.

In some embodiments, there is provided a method of delivering one or more mRNA into a cell in an individual comprising administering to the individual a composition comprising an mRNA delivery complex or nanoparticle as described herein, wherein the complex or nanoparticle comprises the one or more mRNA and a CPP selected from the group consisting of PEP-1 peptides, PEP-2 peptides, PEP-3 peptides, VEPEP-3 peptides, VEPEP-6 peptides, VEPEP-9 peptides, and ADGN-100 peptides. In some embodiments, the composition is administered to the individual via an intravenous, intraarterial, intraperitoneal, intravesicular, subcutaneous, intrathecal, intracranial, intracerebral, intracerebroventricular, intrapulmonary, intramuscular, intratracheal, intraocular, ophthalmic, intraportal, transdermal, intradermal, oral, sublingual, topical, or inhalation route. In some embodiments, the composition is administered to the individual via an intravenous route. In some embodiments, the composition is administered to the individual via a subcutaneous route. In some embodiments, the cell is present in an organ or tissue including lung, liver, brain, kidney, heart, spleen, blood, pancreas, muscle, bone marrow, and intestine. In some embodiments, the cell is present in the lung, liver, kidney, or spleen of the individual. In some embodiments, the cell is an immune cell, such as a granulocyte, a mast cell, a monocyte, a dendritic cell, a B cell, a T cell, or a natural killer cell. In some embodiments, the cell is a fibroblast. In some embodiments, the cell is a muscle cell. In some embodiments, the cell is a cardiac cell. In some embodiments, the cell is a hepatocyte. In some embodiments, the cell is a human lung progenitor cell (LPC). In some embodiments, the cell is a neuronal cell. In some embodiments, the individual has, or is at risk of developing, a disease, and the mRNA is useful for the treatment of the disease. In some embodiments, the complex or nanoparticle further comprises a RNAi. In some embodiments, the RNAi is useful for the treatment of the disease. In some embodiments, the composition is a pharmaceutical composition, and further comprises a pharmaceutically acceptable carrier. In some embodiments, the individual is a mammal. In some embodiments, the individual is human.

In some embodiments, there is provided a method of delivering a transgene into a cell comprising contacting the cell with an mRNA delivery complex or nanoparticle as described herein, wherein the mRNA delivery complex or nanoparticle comprises the transgene packaged in an mRNA and a CPP comprising the amino acid sequence of a PEP-1 peptide, a PEP-2 peptide, a VEPEP-3 peptide, a VEPEP-6 peptide, a VEPEP-9 peptide, or an ADGN-100 peptide. In some embodiments, the VEPEP-3 peptide comprises the amino acid sequence of any one of SEQ ID NOs: 1-14, 75, and 76. In some embodiments, the VEPEP-6 peptide comprises the amino acid sequence of any one of SEQ ID NOs: 15-40, and 77. In some embodiments, the VEPEP-9 peptide comprises the amino acid sequence of any one of SEQ ID NOs: 41-52, and 78. In some embodiments, the ADGN-100 peptide comprises the amino acid sequence of any one of SEQ ID NOs: 53-70, 79, and 80. In some embodiments, the mRNA encodes a protein, such as a therapeutic protein. In some embodiments, the mRNA encodes a CAR. In some embodiments, the complex or nanoparticle comprises a plurality of mRNA encoding a plurality of protein. In some embodiments, the complex or nanoparticle comprises a plurality of mRNA encoding a single protein. In some embodiments, the complex or nanoparticle comprises a single mRNA encoding a first protein and a second protein. In some embodiments, the complex or nanoparticle further comprises a RNAi such as siRNA, such as an RNAi targeting an endogenous gene, e.g., a disease-associated endogenous gene. In some embodiments, the RNAi targets an exogenous gene. In some embodiments, the RNAi is a therapeutic RNAi targeting an endogenous gene involved in a disease or condition, and the protein is a therapeutic protein useful for treating the disease or condition. In some embodiments, the complex or nanoparticle comprises a therapeutic mRNA and a therapeutic RNAi, wherein the therapeutic RNAi targets a disease-associated form of the endogenous gene (e.g., a gene encoding a mutant protein, or a gene resulting in abnormal expression of a protein), and the therapeutic mRNA corresponds to a therapeutic form of the endogenous gene (e.g., the second transgene encodes a wild-type or functional form of the mutant protein, or the second transgene results in normal expression of the protein). In some embodiments, the contacting of the cell with the complex or nanoparticle is carried out in vivo. In some embodiments, the contacting of the cell with the complex or nanoparticle is carried out ex vivo. In some embodiments, the contacting of the cell with the complex or nanoparticle is carried out in vitro. In some embodiments, the cell is an immortalized cell, such as a cell from a cell line. In some embodiments, the cell is a primary cell, such as a cell from an individual. In some embodiments, the cell is an immune cell, such as a granulocyte, a mast cell, a monocyte, a dendritic cell, a B cell, a T cell, or a natural killer cell. In some embodiments, the T cell is an immortalized T cell, such as a T cell from a T cell line. In some embodiments, the T cell is a primary T cell, such as a T cell of an individual. In some embodiments, the cell is a fibroblast. In some embodiments, the fibroblast is a primary fibroblast, such as a fibroblast of an individual. In some embodiments, the cell is a muscle cell. In some embodiments, the cell is a cardiac cell. In some embodiments, the cell is a hepatocyte. In some embodiments, the hepatocyte is a primary hepatocyte, such as a hepatocyte of an individual. In some embodiments, the cell is a human lung progenitor cell (LPC). In some embodiments, the cell is a neuronal cell. In some embodiments, the mRNA is useful for the treatment of a disease, such as any of the diseases to be treated described herein (e.g., cancer, diabetes, autoimmune diseases, inflammatory diseases, fibrotic diseases, viral infectious diseases, hereditary diseases, ocular diseases, and aging and degenerative diseases). In some embodiments, the mRNA and/or RNAi is useful for modulating a protein involved in a disease, such as any of the diseases to be treated described herein (e.g., cancer, diabetes, autoimmune diseases, inflammatory diseases, fibrotic diseases, viral infectious diseases, hereditary diseases, ocular diseases, and aging and degenerative diseases). In some embodiments, the cell-penetrating peptide is an ADGN-100 peptide or a VEPEP-3 peptide.

In some embodiments, here is provided a method of delivering a complex or nanoparticle comprising an mRNA and a cell-penetrating peptide to a subject, wherein the complex or nanoparticle targets a local tissue, organ or cell. In some embodiments, the local tissue, organ or cell is a disease region. In some embodiments, the local tissue, organ or cell is not a disease region. In some embodiments, the local delivery is mediated via a targeting moiety. In some embodiments, the local delivery is mediated via the cell-penetrating peptide. In some embodiments, the local delivery is mediated via the local administration. In some embodiments, the local delivery is mediated via a specific device described herein. In some embodiments, the local delivery is mediated via a combination of the mechanisms described herein.

In some embodiments, there is provided a method of treating a cancer in an individual, comprising administering one or more mRNA into the individual, wherein the mRNA encodes a tumor suppressor protein. In some embodiments, the tumor suppressor protein corresponds to a tumor-suppressor gene. In some embodiments, the corresponding tumor-suppressor gene includes, without limitation, PTEN, Retinoblastoma RB (or RB1). TP53, TP63, TP73, CDKN2A (INK4A), CDKN1B, CDKN1C, DLD/NP1, HEPACAM, SDHB, SDHD, SFRP1, TCF21, TIG1, MLH1, MSH2, MSH6, WT1, WT2, NF1, NF2N, VHL, KLF4, pVHL, APC, CD95, ST5, YPEL3, ST7, APC, MADR2, BRCA1, BRCA2, Patched, TSC1, TSC2, PALB2, ST14, or VHL. In some embodiments, the tumor suppressor gene is selected from PB1, TSC1, TSC2, BRCA1, BRCA2, PTEN and TP53. In some embodiments, the one or more mRNA is delivered with an mRNA delivery complex or nanoparticle as described herein.

In some embodiments, there is provided a method of treating a cancer in an individual, comprising administering one or more mRNA into the individual, wherein the mRNA encodes a tumor suppressor protein PTEN. In some embodiments, the tumor suppressor protein PTEN is encoded by a human PTEN sequence. In some embodiments, the mRNA comprises a sequence selected from the group consisting of sequences with accession number of BC005821, JF268690, U92436, CR450306, AK024986, AK313581, U96180, and U93051 and NM_000314 in NCBI GenBank. In some embodiments, the cancer is selected from pancreatic cancer, ovarian cancer, prostate cancer, and glioblastoma. In some embodiments, the cancer is pancreatic cancer. In some embodiments, the individual has an aberration in a gene encoding the tumor suppressor protein. In some embodiments, the mRNA is delivered intravenously or subcutaneously. In some embodiments, the mRNA is complexed with a cell-penetrating peptide when delivered into the individual. In some embodiments, the cell-penetrating peptide is selected from the group consisting of CADY, PEP-1, PEP-2, MPG, VEPEP-3 peptides (used herein interchangeably with ADGN-103 peptides), VEPEP-4 peptides (used herein interchangeably with ADGN-104 peptides), VEPEP-5 peptides (used herein interchangeably with ADGN-105 peptides), VEPEP-6 peptides (used herein interchangeably with ADGN-106 peptides), VEPEP-9 peptides (used herein interchangeably with ADGN-109 peptides), and ADGN-100 peptides. In some embodiments, the cell-penetrating peptide is selected from ADGN 106 peptides and ADGN-100 peptides. In some embodiments, the mRNA is delivered about once a week or once every five days. In some embodiments, the dose of the mRNA for each administration in an individual is about 0.001 mg/kg to about 10 mg/kg (e.g., about 0.01 mg/kg to about 1 mg/kg, about 0.02 mg/kg to about 0.1 mg/kg). In some embodiments, the dose of the mRNA for each administration in an individual is about 0.03 mg/m² to about 40 mg/m² (e.g., about 0.4 mg/m² to about 40 mg/m², about 0.8 mg/M2 to about 4 mg/m²). In some embodiments, the individual is a human.

In some embodiments, there is provided a method of treating a cancer in an individual, comprising administering one or more mRNA into the individual, wherein the mRNA encodes a tumor suppressor protein p53. In some embodiments, the tumor suppressor protein p53 is encoded by a human TP53 sequence. In some embodiments, the mRNA comprises a sequence selected from the group consisting of sequences with accession number of AF052180, NM_000546, AY429684, BT019622, AK223026, DQ186652, DQ186651. DQ186650, DQ186649, DQ186648, DQ263704, DQ286964, DQ191317, DQ401704, AF307851, AM076972, AM076971, AM076970, DQ485152, BC003596, DQ648887, DQ648886, DQ648885, DQ648884, AK225838, M14694, M14695, EF101869, EF101868, EF101867, X01405, AK312568, NM_001126117, NM_001126116, NM_001126115, NM_001126114, NM_001126113, NM_001126112, FJ207420, X60020, X60019, X60018, X60017, X60016, X60015, X60014, X60013, X60011, X60012, X60010, X02469, S66666, AB082923. NM_001126118, JN900492, NM_001276699, NM_001276698, NM_001276697, NM_001276761, NM_001276760, NM_001276696, and NM_001276695 in NCBI GenBank. In some embodiments, the cancer is selected from pancreatic cancer, ovanan cancer, prostate cancer, and glioblastoma. In some embodiments, the cancer is pancreatic cancer. In some embodiments, the individual has an aberration in a gene encoding the tumor suppressor protein. In some embodiments, the mRNA is delivered intravenously or subcutaneously. In some embodiments, the mRNA is complexed with a cell-penetrating peptide when delivered into the individual. In some embodiments, the cell-penetrating peptide is selected from the group consisting of CADY, PEP-1, PEP-2, MPG, VEPEP-3 peptides (used herein interchangeably with ADGN-103 peptides), VEPEP-4 peptides (used herein interchangeably with ADGN-104 peptides), VEPEP-5 peptides (used herein interchangeably with ADGN-105 peptides), VEPEP-6 peptides (used herein interchangeably with ADGN-106 peptides), VEPEP-9 peptides (used herein interchangeably with ADGN-109 peptides), and ADGN-100 peptides. In some embodiments, the cell-penetrating peptide is selected from ADGN 106 peptides and ADGN-100 peptides. In some embodiments, the mRNA is delivered about once a week or once every five days. In some embodiments, the dose of the mRNA for each administration in an individual is about 0.001 mg/kg to about 10 mg/kg (e.g., about 0.01 mg/kg to about 1 mg/kg, about 0.02 mg/kg to about 0.1 mg/kg). In some embodiments, the dose of the mRNA for each administration in an individual is about 0.03 mg/m² to about 40 mg/m² (e.g., about 0.4 mg/m² to about 40 mg/m², about 0.8 mg/M2 to about 4 mg/m²). In some embodiments, the individual is a human.

In some embodiments, there is provided a method of treating a cancer in an individual, comprising administering one or more mRNA into the individual, wherein the mRNA encodes a tumor suppressor protein BRCA1. In some embodiments, the tumor suppressor protein BRCA1 is encoded by a human BRCA1 sequence. In some embodiments, the mRNA comprises a sequence selected from the group consisting of a sequence with with accession number of NM_007294, NM_007297, NM_007298, NM 007304, NM 007299, NM_007300, BC046142, BC062429, BC072418, AY354539, AY751490, BC085615, BC106746, BC106745, BC114511, BC115037, U14680, AK293762, U68041, BC030969, BC012577, AK316200, DQ363751, DQ333387, DQ333386, Y08864, JN686490, AB621825, BC038947, U64805, and AF005068 in NCBI GenBank. In some embodiments, the cancer is selected from pancreatic cancer, ovarian cancer, prostate cancer, and glioblastoma. In some embodiments, the cancer is pancreatic cancer. In some embodiments, the individual has an aberration in a gene encoding the tumor suppressor protein. In some embodiments, the mRNA is delivered intravenously or subcutaneously. In some embodiments, the mRNA is complexed with a cell-penetrating peptide when delivered into the individual. In some embodiments, the cell-penetrating peptide is selected from the group consisting of CADY, PEP-1, PEP-2, MPG, VEPEP-3 peptides (used herein interchangeably with ADGN-103 peptides), VEPEP-4 peptides (used herein interchangeably with ADGN-104 peptides), VEPEP-5 peptides (used herein interchangeably with ADGN-105 peptides). VEPEP-6 peptides (used herein interchangeably with ADGN-106 peptides), VEPEP-9 peptides (used herein interchangeably with ADGN-109 peptides), and ADGN-100 peptides. In some embodiments, the cell-penetrating peptide is selected from ADGN 106 peptides and ADGN-100 peptides. In some embodiments, the mRNA is delivered about once a week or once every five days. In some embodiments, the dose of the mRNA for each administration in an individual is about 0.001 mg/kg to about 10 mg/kg (e.g., about 0.01 mg/kg to about 1 mg/kg, about 0.02 mg/kg to about 0.1 mg/kg). In some embodiments, the dose of the mRNA for each administration in an individual is about 0.03 mg/m² to about 40 mg/m² (e.g., about 0.4 mg/m² to about 40 mg/m², about 0.8 mg/m² to about 4 mg/m²). In some embodiments, the individual is a human.

In some embodiments, there is provided a method of treating a cancer in an individual, comprising administering one or more mRNA into the individual, wherein the mRNA encodes a tumor suppressor protein BRCA2. In some embodiments, the tumor suppressor protein BRCA2 is encoded by a human BRCA2 sequence. In some embodiments, the mRNA comprises a sequence selected from the group consisting of a sequence with with accession number of BC047568, NM_000059, DQ897648, BC026160 in NCBI GenBank. In some embodiments, the cancer is selected from pancreatic cancer, ovarian cancer, prostate cancer, and glioblastoma. In some embodiments, the cancer is pancreatic cancer. In some embodiments, the individual has an aberration in a gene encoding the tumor suppressor protein. In some embodiments, the mRNA is delivered intravenously or subcutaneously. In some embodiments, the mRNA is complexed with a cell-penetrating peptide when delivered into the individual. In some embodiments, the cell-penetrating peptide is selected from the group consisting of CADY, PEP-1, PEP-2, MPG, VEPEP-3 peptides (used herein interchangeably with ADGN-103 peptides), VEPEP-4 peptides (used herein interchangeably with ADGN-104 peptides), VEPEP-5 peptides (used herein interchangeably with ADGN-105 peptides), VEPEP-6 peptides (used herein interchangeably with ADGN-106 peptides), VEPEP-9 peptides (used herein interchangeably with ADGN-109 peptides), and ADGN-100 peptides. In some embodiments, the cell-penetrating peptide is selected from ADGN 106 peptides and ADGN-100 peptides. In some embodiments, the mRNA is delivered about once a week or once every five days. In some embodiments, the dose of the mRNA for each administration in an individual is about 0.001 mg/kg to about 10 mg/kg (e.g., about 0.01 mg/kg to about 1 mg/kg, about 0.02 mg/kg to about 0.1 mg/kg). In some embodiments, the dose of the mRNA for each administration in an individual is about 0.03 mg/m² to about 40 mg/m² (e.g., about 0.4 mg/m² to about 40 mg/m², about 0.8 mg/m² to about 4 mg/m²). In some embodiments, the individual is a human.

In some embodiments, there is provided a method of treating a cancer in an individual, comprising administering one or more mRNA into the individual, wherein the mRNA encodes a tumor suppressor protein TSC1. In some embodiments, the tumor suppressor protein TSC1 is encoded by a human TSC1 sequence. In some embodiments, the mRNA comprises a sequence selected from the group consisting of a sequence with with accession number of BC047772, NM_000368, BC070032, AB190910, BC108668, BC121000, NM_001162427, NM_001162426, D87683, and AF013168 in NCBI GenBank. In some embodiments, the cancer is selected from pancreatic cancer, ovarian cancer, prostate cancer, and glioblastoma. In some embodiments, the cancer is pancreatic cancer. In some embodiments, the individual has an aberration in a gene encoding the tumor suppressor protein. In some embodiments, the mRNA is delivered intravenously or subcutaneously. In some embodiments, the mRNA is complexed with a cell-penetrating peptide when delivered into the individual. In some embodiments, the cell-penetrating peptide is selected from the group consisting of CADY, PEP-1, PEP-2, MPG, VEPEP-3 peptides (used herein interchangeably with ADGN-103 peptides), VEPEP-4 peptides (used herein interchangeably with ADGN-104 peptides), VEPEP-5 peptides (used herein interchangeably with ADGN-105 peptides), VEPEP-6 peptides (used herein interchangeably with ADGN-106 peptides), VEPEP-9 peptides (used herein interchangeably with ADGN-109 peptides), and ADGN-100 peptides. In some embodiments, the cell-penetrating peptide is selected from ADGN 106 peptides and ADGN-100 peptides. In some embodiments, the mRNA is delivered about once a week or once every five days. In some embodiments, the dose of the mRNA for each administration in an individual is about 0.001 mg/kg to about 10 mg/kg (e.g., about 0.01 mg/kg to about 1 mg/kg, about 0.02 mg/kg to about 0.1 mg/kg). In some embodiments, the dose of the mRNA for each administration in an individual is about 0.03 mg/m² to about 40 mg/m² (e.g., about 0.4 mg/m² to about 40 mg/m², about 0.8 mg/m² to about 4 mg/m²). In some embodiments, the individual is a human.

In some embodiments, there is provided a method of treating a cancer in an individual, comprising administering one or more mRNA into the individual, wherein the mRNA encodes a tumor suppressor protein TSC2. In some embodiments, the tumor suppressor protein TSC2 is encoded by a human TSC2 sequence. In some embodiments, the mRNA comprises a sequence selected from the group consisting of a sequence with accession number of BC046929, BX647816, AK125096, NM_000548, AB210000, NM_001077183, BC150300, BC025364, NM_001114382, AK094152, AK299343, AK295728, AK295672, AK294548, and X75621 in NCBI GenBank. In some embodiments, the cancer is selected from pancreatic cancer, ovarian cancer, prostate cancer, and glioblastoma. In some embodiments, the cancer is pancreatic cancer. In some embodiments, the individual has an aberration in a gene encoding the tumor suppressor protein. In some embodiments, the mRNA is delivered intravenously or subcutaneously. In some embodiments, the mRNA is complexed with a cell-penetrating peptide when delivered into the individual. In some embodiments, the cell-penetrating peptide is selected from the group consisting of CADY, PEP-1, PEP-2, MPG, VEPEP-3 peptides (used herein interchangeably with ADGN-103 peptides), VEPEP-4 peptides (used herein interchangeably with ADGN-104 peptides), VEPEP-5 peptides (used herein interchangeably with ADGN-105 peptides), VEPEP-6 peptides (used herein interchangeably with ADGN-106 peptides), VEPEP-9 peptides (used herein interchangeably with ADGN-109 peptides), and ADGN-100 peptides. In some embodiments, the cell-penetrating peptide is selected from ADGN 106 peptides and ADGN-100 peptides. In some embodiments, the mRNA is delivered about once a week or once every five days. In some embodiments, the dose of the mRNA for each administration in an individual is about 0.001 mg/kg to about 10 mg/kg (e.g., about 0.01 mg/kg to about 1 mg/kg, about 0.02 mg/kg to about 0.1 mg/kg). In some embodiments, the dose of the mRNA for each administration in an individual is about 0.03 mg/m² to about 40 mg/m² (e.g., about 0.4 mg/m² to about 40 mg/m², about 0.8 mg/m² to about 4 mg/m²). In some embodiments, the individual is a human.

In some embodiments, there is provided a method of treating a cancer in an individual, comprising administering one or more mRNA into the individual, wherein the mRNA encodes a tumor suppressor protein Retinoblastoma 1 (RB1). In some embodiments, the tumor suppressor protein RB1 is encoded by a human RB sequence. In some embodiments, the mRNA comprises a sequence selected from the group consisting of a sequence with accession number of NM_000321. AY429568. AB208788, M19701, AK291258, L41870, AK307730, AK307125, AK300284, AK299179, M33647, M15400, M28419, BC039060, BC040540, and AF043224 in NCBI GenBank. In some embodiments, the cancer is selected from pancreatic cancer, ovarian cancer, prostate cancer, and glioblastoma. In some embodiments, the cancer is pancreatic cancer. In some embodiments, the individual has an aberration in a gene encoding the tumor suppressor protein. In some embodiments, the mRNA is delivered intravenously or subcutaneously. In some embodiments, the mRNA is complexed with a cell-penetrating peptide when delivered into the individual. In some embodiments, the cell-penetrating peptide is selected from the group consisting of CADY, PEP-1, PEP-2, MPG, VEPEP-3 peptides (used herein interchangeably with ADGN-103 peptides), VEPEP-4 peptides (used herein interchangeably with ADGN-104 peptides), VEPEP-5 peptides (used herein interchangeably with ADGN-105 peptides), VEPEP-6 peptides (used herein interchangeably with ADGN-106 peptides), VEPEP-9 peptides (used herein interchangeably with ADGN-109 peptides), and ADGN-100 peptides. In some embodiments, the cell-penetrating peptide is selected from ADGN 106 peptides and ADGN-100 peptides. In some embodiments, the mRNA is delivered about once a week or once every five days. In some embodiments, the dose of the mRNA for each administration in an individual is about 0.001 mg/kg to about 10 mg/kg (e.g., about 0.01 mg/kg to about 1 mg/kg, about 0.02 mg/kg to about 0.1 mg/kg). In some embodiments, the dose of the mRNA for each administration in an individual is about 0.03 mg/m² to about 40 mg/m² (e.g., about 0.4 mg/m² to about 40 mg/m² about 0.8 mg/m² to about 4 mg/m²). In some embodiments, the individual is a human.

In some embodiments, there is provided a method of treating a cancer in an individual, comprising administering one or more RNAi (e.g., siRNA) into the individual, wherein the RNAi (e.g., siRNA) comprises an RNAi (e.g., siRNA) targeting KRAS. In some embodiments, the RNAi (e.g., siRNA) targets a mutant form of KRAS. In some embodiments, the RNAi (e.g., siRNA) specifically targets a mutant form of KRAS but not the wildtype form of KRAS. In some embodiments, the mutant form comprises an aberration of KRAS, wherein the aberration of KRAS comprises a mutation on codon 12, 13, 17, 34 or 61 of KRAS. In some embodiments, the mutant form comprises an aberration of KRAS, wherein the aberration of KRAS is selected from the group consisting of G12C, G12S, G12R, G12F, G12L, G12N, G12A, G12D, G12S, G12V, G13C, G13S, G13R, G13A, G13D, G13V, G13P, S17G, P34S, Q61E, Q61K, Q61L, Q61R, Q61P. Q61H, K117N, A146P, A146T and A146V. In some embodiments, the mutant form comprises an aberration of KRAS, wherein the aberration of KRAS is selected from the group consisting of G12C, G12S, G12R, G12F, G12L, G12N, G12A, G12D, G12V, G13C, G13S, G13D, G13V, G13P, S17G, P34S, Q61K, Q61L, Q61R. and Q61H. In some embodiments, the mutant form comprises an aberration of KRAS, wherein the aberration of KRAS is selected from the group consisting of G12C, G12R, G12S, G12A, G12D, G12V, G13C, G13R, G13S, G13A, G13D, G13V, Q61K. Q61L, Q61R, Q61H, K K117N, A146P, A146T and A146V. In some embodiments, the mutant form comprises an aberration of KRAS, wherein the aberration of KRAS is selected from the group consisting of KRAS G12A, G12C, G12D, G12R, G12S, G12V, G13A, G13C, G13D, G13R, G13S, G13V, Q61E. Q61H, Q61K, Q61L, Q61P, and Q61R. In some embodiments, the aberration of KRAS is selected from the group consisting of KRAS G12C, G12D, G12R, G12S, G12V and G13D. In some embodiments, the aberration of KRAS comprises G12C. In some embodiments, the aberration of KRAS comprises G12D. In some embodiments, the aberration of KRAS comprises Q61K. In some embodiments, the aberration of KRAS comprises G12C and G12D. In some embodiments, the aberration of KRAS comprises G12C and Q61K. In some embodiments, the aberration of KRAS comprises G12D and Q61K. In some embodiments, the aberration of KRAS comprises G12C, G12D and Q61K. In some embodiments, the RNAi (e.g., siRNA) comprises one or more sequences of 5′-GUUGGAGCUUGUGGCGUAGTT-3′ (sense) (SEQ ID NO: 83), 5′-CUACGCCACCAGCUCCAACTT-3 (anti-sense) (SEQ ID NO: 84), 5′-GAAGUGCAUACACCGAGACTT-3′ (sense) (SEQ ID NO: 86), 5′-GUCUCGGUGUAGCACUUCTT-3′ (anti-sense) (SEQ ID NO: 87), 5′-GUUGGAGCUGUUGGCGUAGTT-3′ (sense) (SEQ ID NO: 88) and/or 5′-CUACGCCAACAGCUCCAACTT-3′ (anti-sense) (SEQ ID NO: 89). In some embodiments, the RNAi (e.g., siRNA) comprises a nucleic acid sequence selected from sequences with SEQ ID NOS: 83, 84, 86-89. In some embodiments, the cancer is selected from pancreatic cancer, ovarian cancer, prostate cancer, and glioblastoma. In some embodiments, the cancer is pancreatic cancer. In some embodiments, the individual has an aberration in the oncogene. In some embodiments, the RNAi is delivered intravenously or subcutaneously. In some embodiments, the RNAi is complexed with a cell-penetrating peptide when delivered into the individual. In some embodiments, the cell-penetrating peptide is selected from the group consisting of CADY, PEP-1, PEP-2, MPG, VEPEP-3 peptides (used herein interchangeably with ADGN-103 peptides), VEPEP-4 peptides (used herein interchangeably with ADGN-104 peptides), VEPEP-5 peptides (used herein interchangeably with ADGN-105 peptides), VEPEP-6 peptides (used herein interchangeably with ADGN-106 peptides), VEPEP-9 peptides (used herein interchangeably with ADGN-109 peptides), and ADGN-100 peptides. In some embodiments, the cell-penetrating peptide is selected from ADGN 106 peptides and ADGN-100 peptides. In some embodiments, the RNAi is delivered about once a week or once every five days. In some embodiments, the dose of the RNAi for each administration in an individual is about 0.001 mg/kg to about 10 mg/kg (e.g., about 0.01 mg/kg to about 1 mg/kg, about 0.02 mg/kg to about 0.1 mg/kg). In some embodiments, the dose of the RNAi (for each administration in an individual is about 0.03 mg/m² to about 40 mg/m² (e.g., about 0.4 mg/m² to about 40 mg/m², about 0.8 mg/m² to about 4 mg/m²). In some embodiments, the individual is a human.

In some embodiments, there is provided a method of treating a disease or condition in an individual, comprising administering one or more mRNA into the individual, wherein the mRNA encodes a therapeutic protein or a recombinant form thereof. In some embodiments, the therapeutic protein is selected from the group consisting of alpha 1 antitrypsin, frataxin, insulin, growth hormone (somatotropin), growth factors, hormones, dystrophin, insulin-like growth factor 1 (IGF1), factor VIII, factor IX, antithrombin III, protein C, β-Gluco-cerebrosidase, Alglucosidase-α, α-1-iduronidase, Iduronate-2-sulphatase, Galsulphase, human α-galactosidase A, α-1-Proteinase inhibitor, lactase, pancreatic enzymes (including lipase, amylase, and protease), Adenosine deaminase, and albumin. In some embodiments, the therapeutic protein is Factor VIII. In some embodiments, the therapeutic protein is administered intravenously or subcutaneously. In some embodiments, the mRNA is delivered intravenously or subcutaneously. In some embodiments, the mRNA is complexed with a cell-penetrating peptide when delivered into the individual. In some embodiments, the cell-penetrating peptide is selected from the group consisting of CADY, PEP-1, PEP-2, MPG, VEPEP-3 peptides (used herein interchangeably with ADGN-103 peptides), VEPEP-4 peptides (used herein interchangeably with ADGN-104 peptides), VEPEP-5 peptides (used herein interchangeably with ADGN-105 peptides), VEPEP-6 peptides (used herein interchangeably with ADGN-106 peptides), VEPEP-9 peptides (used herein interchangeably with ADGN-109 peptides), and ADGN-100 peptides. In some embodiments, the cell-penetrating peptide is selected from ADGN 106 peptides and ADGN-100 peptides. In some embodiments, the mRNA is delivered at a frequency of about or less than once a week, once every two weeks, once every three weeks or once a month. In some embodiments, the dose of the mRNA for each administration in an individual is about 0.001 mg/kg to about 10 mg/kg (e.g., about 0.01 mg/kg to about 1 mg/kg, about 0.02 mg/kg to about 0.1 mg/kg). In some embodiments, the dose of the mRNA for each administration in an individual is about 0.03 mg/m² to about 40 mg/m² (e.g., about 0.4 mg/m² to about 40 mg/m², about 0.8 mg/m² to about 4 mg/m²). In some embodiments, the individual is a human.

### Targeting Moiety

In some embodiments, here is provided a method of delivering a complex or nanoparticle comprising an mRNA and a cell-penetrating peptide to a subject, wherein the complex or nanoparticle targets a local tissue, organ or cell via a targeting moiety. In some embodiments, an mRNA delivery complex or nanoparticle described herein comprises a cell-penetrating peptide, wherein cell-penetrating peptide is linked to a targeting moiety. In some embodiments, the targeting moiety o described herein targets the mRNA delivery complex to a tissue or a specific cell type. In some embodiments, the tissue is a tissue in need of treatment. In some embodiments, the targeting moiety targets the mRNA delivery complex to a tissue or cell that can be treated by the mRNA. In some embodiments, at least some of the peripheral cell-penetrating peptides in the surface layer are linked to a targeting moiety. In some embodiments, the linkage is covalent. In some embodiments, the covalent linkage is by chemical coupling. In some embodiments, the covalent linkage is by genetic methods.

### Cell-penetrating Peptide

In some embodiments, the complex or nanoparticle comprises a cell-penetrating peptide, wherein the cell-penetrating peptide preferentially targets a specific tissue, organ or cell in a subject.

### Local Administration

In some embodiments, there is provided a method of delivering a complex or nanoparticle comprising an mRNA and a cell-penetrating peptide to a subject, wherein the complex or nanoparticle is administered to the subject via a route selected from intraperitoneal, intravesicular, subcutaneous, intrathecal, intracranial, intracoronary, intracerebral, intracerebroventricular, intrapulmonary, intramuscular, intratracheal (e.g., via non-surgical intratracheal, e.g., via nebulization or instillation), intraocular, ophthalmic, intraportal, transdermal, intradermal, oral, sublingual, topical, or inhalation route. In some embodiments, here is provided a method of delivering a complex or nanoparticle comprising an mRNA and a cell-penetrating peptide to a subject, wherein the complex or nanoparticle is administered to the individual via topical route. In some embodiments, here is provided a method of delivering a complex or nanoparticle comprising an mRNA and a cell-penetrating peptide to a subject, wherein the complex or nanoparticle is administered to the individual via systemic route (e.g., intravenously).

In some embodiments, the complex or nanopartical is administered into a subject through a catheter with a needle (such as a deployable needle), wherein the needle contains the complex or nanoparticle. For example, catheters carrying needles capable of delivering therapeutic and other agents deep into the adventitial layer surrounding blood vessel lumens have been described in U.S. Pat. Nos. 6,547,303, 6,860,867 and U.S. Patent Application Publication Nos. 2007/0106257, 201010305546, and 200910142306, the content of each of these are specifically incorporated herein by reference in their entirety. In some embodiments, the needle is deployable. The catheter can be advanced intravascularly to a target injection site (which may or may not be a disease region) in a blood vessel. The needle in the catheter is advanced through the blood vessel wall so that an aperture on the needle is positioned in the desired region (for example the perivascular region), and the complex or nanoparticle compositions can be injected through the aperture of the needled into the desired region.

For example, in some embodiments there is provided a method of delivering a complex or nanoparticle comprising an mRNA and a cell-penetrating peptide to a blood vessel, wherein the method comprises injecting (for example via a catheter with a needle) into the tissue surrounding the blood vessel wall an effective amount of a complex or nanoparticle comprising an mRNA and a cell-penetrating peptide. In some embodiments, the mRNA encodes a therapeutic protein, for example, a tumor suppressor protein. In some embodiments, there is provided a method of delivering a complex or nanoparticle comprising an mRNA and a cell-penetrating peptide to a blood vessel, wherein the method comprises injecting (for example via a catheter with a needle) into the tissue surrounding the blood vessel wall an effective amount of a complex or nanoparticle comprising an mRNA and a cell-penetrating peptide, wherein the complex or nanoparticle further comprises a RNAi. In some embodiments, the RNAi targets an endogenous gene, for example, a disease associated endogenous gene. In some embodiments, the RNAi targets an exogenous gene. In some embodiments, the complex or nanoparticle is injected at a disease site. In some embodiment, the complex or nanoparticle is injected distal to a disease site (such example at least about any of 1, 2, 3, 4, 5, 6, 7, 8, 9, 10 cm away from the disease site.

In some embodiments, the complex or nanoparticle is injected into the adventitial tissue of the blood vessel. The adventitial tissue is the tissue surrounding the blood vessel, for example the tissue beyond the external elastic lamina of an artery or beyond the tunica media of a vein. The adventitia has a high concentration of lipid. In some embodiments, the complex or nanoparticle is injected into the vasa vasorum region of the adventitia. In some embodiments, the complex or nanoparticle, upon injection, can disperse through the adventitia circumferentially, longitudinally, and/or transmurally from the injection site with respect to the axis of the blood vessel from which the complex or nanoparticle is being injected (herein after referred to as “volumetric distribution”). In some embodiments, the complex or nanoparticle distributes over a distance of at least about 1 cm (for example at least about any of 2 cm, 3 cm, 4 cm, 5 cm, 6 cm, 7 cm, or more) longitudinally and/or at least 1 cm (for example at least about any of 2 cm, 3 cm, 4 cm, 5 cm, 6 cm, 7 cm, or more) radially from the site of injection over a time period no greater than 60 minutes. In some embodiments, a concentration of a complex or nanoparticle measured at all locations at least 2 cm from the delivery site is at least 10% (such as at least about any of 20%, 30%, 40%, or 50%) of the concentration at the delivery site, for example after a period of 60 minutes. In some embodiments, the complex or nanoparticle distributes transmurally throughout the endothelial and intimal layers of the blood vessel, the media, and the muscular layer.

Thus, in some embodiments, there is provided a method of delivering a complex or nanoparticle comprising a mRNA and a CPP to a blood vessel, wherein the method comprises injecting (for example via a catheter with a needle) into the adventitial tissue of the blood vessel wall an effective amount of a composition comprising a complex or nanoparticle comprising a mRNA and a CPP. In some embodiments, there is provided a method of delivering a complex or nanoparticle comprising a mRNA and a CPP to a blood vessel, wherein the method comprises injecting (for example via a catheter with a needle) into the adventitial tissue of the blood vessel an effective amount of a complex or nanoparticle comprising a mRNA and a CPP, wherein the complex or nanoparticle further comprises a RNAi. In some embodiments, the mRNA encodes a therapeutic protein. In some embodiments, the RNAi targets an endogenous gene, for example, a disease-associated endogenous gene. In some embodiments, the RNAi targets an exogenous gene. In some embodiments, there is provided a method of delivering a nanoparticle comprising a mRNA and a CPP to a blood vessel, wherein the method comprises injecting (for example via a catheter with a needle) into the adventitial tissue of the blood vessel wall an effective amount of a composition comprising a complex or nanoparticle comprising a mRNA and a CPP, wherein the average size of the nanoparticle is less than 200 nm. In some embodiments, the complex or nanoparticle is injected at or adjacent to a disease site (such as no more than about 2, 1, or 0.5 cm away from the disease site). In some embodiments, the complex or nanoparticle is injected remotely from a disease site (such example at least about any of 1, 2, 3, 4, 5, 6, 7, 8, 9, or 10 cm away from the disease site). In some embodiments, the nanoparticle composition, upon injection, achieves a volumetric distribution.

The blood vessel described in some embodiments is an artery, such as a coronary artery or a peripheral artery. In some embodiments, the artery is selected from the group consisting of renal artery, cerebral artery, pulmonary artery, and artery in the leg. In some embodiments, the blood vessel is an artery or vein above the knee. In some embodiments, the blood vessel is an artery or vein below the knee. In some embodiments, the blood vessel is a femoral artery. In some embodiments, the blood vessel is a balloon injured artery.

In some embodiments, the blood vessel is an artery selected from any one of the following: abdominal aorta, anterior tibial artery, arch of aorta, arcuate artery, axillary artery, brachial artery, carotid artery, celiac artery, circumflex fibular artery, common hepatic artery, common iliac artery, deep femoral artery, deep palmar arterial arch, dorsal digital artery, dorsal metatarsal artery, external carotid artery, external iliac artery, facial artery, femoral artery, inferior mesenteric artery, internal iliac artery, instestinal artery, lateral inferior genicular artery, lateral superior genicular artery, palmar digital artery, peroneal artery, popliteal artery, posterior tibial artery, profunda femoris artery, pulmonary artery, radial artery, renal artery, splenic artery, subclavian artery, superficial palmar arterial arch, superior mesenteric artery, superior ulnar collateral artery, and ulnar artery.

In some embodiments, the blood vessel is a vein. In some embodiments, the blood vessel is a vein selected from any one of the following: accessory cephalic vein, axillary vein, basilic vein, brachial vein, cephalic vein, common iliac vein, dorsal digital vein, dorsal metatarsal vein, external iliac vein, facial vein, femoral vein, great saphenous vein, hepatic vein, inferior mesenteric vein, inferior vena cava, intermediate antebrachial vein, internal iliac vein, intestinal vein, jugular vein, lateral circumflex femoral vein, left inferior pulmonary vein, left superior pulmonary vein, palmar digital vein, portal vein, posterior tibial vein, renal vein, retromanibular vein, saphenous vein, small saphenous vein, splenic vein, subclavian vein, superior mesenteric vein, and superior vena cava.

In some embodiments, the blood vessel is part of the coronary vasculature (including the arterial and venous vasculature), the cerebral vasculature, the hepatic vasculature, the peripheral vasculature, and the vasculature of other organs and tissue compartments.

In some embodiments, there is provided a method of delivering a complex or nanoparticle comprising an mRNA and a CPP to a blood vessel, wherein the method comprises injecting (for example via a catheter with a needle) periadventitially (i.e., injecting into the periadventitial tissue) to a femoral artery an effective amount of a complex or nanoparticle comprising an mRNA and a CPP. In some embodiments, there is provided a method of delivering a complex or nanoparticle comprising a mRNA and a CPP to a blood vessel, wherein the method comprises injecting (for example via a catheter with a needle) into the periadventitial tissue of the blood vessel an effective amount of a complex or nanoparticle comprising a mRNA and a CPP, wherein the complex or nanoparticle further comprises a RNAi. In some embodiments, the mRNA encodes a therapeutic protein. In some embodiments, the RNAi targets an endogenous gene, for example, a disease-associated endogenous gene. In some embodiments, the RNAi targets an exogenous gene. In some embodiments, there is provided a method of delivering a nanoparticle comprising a mRNA and a CPP to a blood vessel, wherein the method comprises injecting (for example via a catheter with a needle) into the periadventitial tissue of the blood vessel wall an effective amount of a composition comprising a complex or nanoparticle comprising a mRNA and a CPP, wherein the average size of the nanoparticle is less than 200 nm. In some embodiments, the complex or nanoparticle is injected at or adjacent to a disease site (such as no more than about 2, 1, or 0.5 cm away from the disease site). In some embodiments, the complex or nanoparticle is injected remotely from a disease site (such example at least about any of 1, 2, 3, 4, 5, 6, 7, 8, 9, or 10 cm away from the disease site). In some embodiments, the nanoparticle composition, upon injection, achieves a volumetric distribution.

The delivery methods described herein are effective in inhibiting one or more aspects of blood vessel abnormalities, including for example, negative remodeling, vascular fibrosis, restenosis, cell proliferation and migration of cells in the blood vessel, and wound healing. In some embodiments, the method is effective in promoting positive remodeling of the blood vessel.

### Methods of Cell Engineering

In some embodiments, there is provided a method of producing an engineered cell, such as an engineered T cell, comprising a method described herein for delivering one or more mRNA into a cell. In some embodiments, the method is an improvement over previous methods of producing an engineered cell, such as methods involving the use of electroporation or non-CPP-mediated viral transfection. In some embodiments, the improvement includes, without limitation, increasing the efficiency of the method, reducing costs associated with the method, reducing cellular toxicity of the method, and/or reducing the complexity of the method.

It is to be understood that any of the methods described herein can be combined. Thus, for example, a first set of one or more mRNA and a second set of one or more mRNA can be delivered into a cell by combining any of the methods described herein for delivering a plurality of mRNA molecules into a cell. Possible combinations contemplated include combinations of two or more of any of the methods described herein.

### Combination Therapy

A. Combination Therapy of mRNA and RNAi (e.g., siRNA)

Also provided herein are combination therapies for treating a disease or condition discussed herein in an individual comprising a) delivering to the individual an mRNA described herein, and b) delivering to the individual an RNAi (e.g., an siRNA) described herein. For example, there is provided a method of treating a disease or condition in an individual comprising delivering to the individual a) an mRNA encoding a tumor suppressor protein and b) an siRNA targeting an oncogene. In some embodiments, the disease or condition is a cancer (e.g., pancreatic cancer, ovarian cancer, prostate cancer, glioblastoma). In some embodiments, the individual has an aberration in the tumor suppressor protein and/or the oncogene protein. In some embodiments, the tumor suppressor protein is selected from PTEN (i.e., the protein encoded by the PTEN gene) and p53 (i.e., p53 tumor-suppressor protein). In some embodiments, the oncogene is KRAS. In some embodiments, the siRNA specifically targets a mutant form of KRAS, wherein the mutant form of KRAS has an aberration of KRAS selected from G12C, G12D and Q61K. In some embodiments, the siRNA comprises a cocktail of siRNAs comprising two or more siRNAs. In some embodiments, the two or more siRNAs comprises a first siRNA targeting a first mutant form of KRAS, and a second siRNA targeting a second mutant form of KRAS. Exemplary two or more siRNAs are not limited but include a) siRNAs targeting both KRAS G12C and KRAS G12D; b) siRNAs targeting both KRAS G12C and KRAS Q61K; c) siRNAs targeting both KRAS G12D and KRAS Q61K; and d) siRNAs targeting KRAS G12C, KRAS G12D and KRAS Q61K. In one preferred embodiment, the first siRNA targets KRAS G12C, and the second siRNA targets KRAS G12D. In some embodiments, the mRNA and/or the siRNA is complexed with a cell-penetrating peptide when delivered into the individual. In some embodiments, the mRNA and the siRNA is complexed with a same cell-penetrating peptide when delivered into the individual. In some embodiments, the mRNA and the siRNA is complexed with a different cell-penetrating peptide when delivered into the individual. In some embodiments, the mRNA and the siRNA are separately complexed with a cell-penetrating peptide when delivered into the individual. In some embodiments, the mRNA is modified (e.g., wherein at least one modified nucleoside is 5-methoxyuridine (5moU)).

In some embodiments, there is provided a method of treating a cancer in an individual, comprising delivering to the individual a) an mRNA encoding a tumor suppressor protein and b) an siRNA targeting an oncogene. In some embodiments, the cancer is selected from pancreatic cancer, ovarian cancer, prostate cancer, and glioblastoma. In some embodiments, the cancer is pancreatic cancer. In some embodiments, the tumor suppressor protein is PTEN. In some embodiments, the oncogene is KRAS. In some embodiments, the siRNA comprises a cocktail of siRNAs comprising two or more siRNAs. In some embodiments, the two or more siRNAs comprises a first siRNA targeting a first mutant form of KRAS, and a second siRNA targeting a second mutant form of KRAS. Exemplary two or more siRNAs are not limited but include a) siRNAs targeting both KRAS G12C and KRAS G12D; b) siRNAs targeting both KRAS G12C and KRAS Q61K; c) siRNAs targeting both KRAS G12D and KRAS Q61K; and d) siRNAs targeting KRAS G12C, KRAS G12D and KRAS Q61K. In one preferred embodiment, the first siRNA targets KRAS G12C, and the second siRNA targets KRAS G12D. In some embodiments, the individual has an aberration in a gene encoding the tumor suppressor protein. In some embodiments, the individual has an aberration in the oncogene. In some embodiments, the individual has an aberration in a gene encoding the tumor suppressor protein and the oncogene. In some embodiments, the mRNA encoding the tumor suppressor protein and the siRNA targeting the oncogene are delivered simultaneously. In some embodiments, the mRNA and the siRNA are delivered concurrently. In some embodiments, the mRNA and/or the siRNA are delivered intravenously. In some embodiments, the mRNA and/or the siRNA are complexed with a cell-penetrating peptide when delivered into the individual. In some embodiments, the cell-penetrating peptide is selected from the group consisting of CADY. PEP-1, PEP-2, MPG, VEPEP-3 peptides (used herein interchangeably with ADGN-103 peptides), VEPEP-4 peptides (used herein interchangeably with ADGN-104 peptides), VEPEP-5 peptides (used herein interchangeably with ADGN-105 peptides), VEPEP-6 peptides (used herein interchangeably with ADGN-106 peptides), VEPEP-9 peptides (used herein interchangeably with ADGN-109 peptides), and ADGN-100 peptides. In some embodiments, the cell-penetrating peptide is selected from ADGN 106 peptides and ADGN-100 peptides. In some embodiments, the mRNA (e.g., the mRNA encoding PTEN) and/or the siRNA (e.g., the siRNA targeting KRAS) are delivered about once a week or once every five days. In some embodiments, the dose of the mRNA (e.g., the mRNA encoding PTEN) for each administration in an individual is about 0.001 mg/kg to about 10 mg/kg (e.g., about 0.01 mg/kg to about 1 mg/kg, about 0.02 mg/kg to about 0.1 mg/kg). In some embodiments, the dose of the siRNA (e.g., the siRNA targeting KRAS) for each administration in an individual is about 0.001 mg/kg to about 10 mg/kg (e.g., about 0.01 mg/kg to about 1 mg/kg, about 0.02 mg/kg to about 0.1 mg/kg). In some embodiments, the dose of the mRNA (e.g., the mRNA encoding PTEN) for each administration in an individual is about 0.03 mg/m² to about 40 mg/m² (e.g., about 0.4 mg/m² to about 40 mg/m², about 0.8 mg/m² to about 4 mg/m²). In some embodiments, the dose of the siRNA (e.g., the siRNA targeting KRAS) for each administration in an individual is about 0.03 mg/m² to about 400 mg/m² (e.g., about 0.4 mg/m² to about 40 mg/m², about 0.8 mg/m² to about 4 mg/m²). In some embodiments, the individual is a human.

In some embodiments, there is provided a method of treating a cancer in an individual, comprising delivering to the individual a) an mRNA encoding a tumor suppressor protein and b) an siRNA targeting an oncogene, wherein the tumor suppressor protein is PTEN. In some embodiments, there is provided a method of treating a cancer in an individual, comprising delivering to the individual a) an mRNA encoding a tumor suppressor protein and b) an siRNA targeting an oncogene, wherein the oncogene is KRAS. In some embodiments, the cancer is selected from pancreatic cancer, ovarian cancer, prostate cancer, and glioblastoma. In some embodiments, the cancer is pancreatic cancer. In some embodiments, the oncogene is KRAS. In some embodiments, the siRNA comprises a cocktail of siRNAs comprising two or more siRNAs. In some embodiments, the two or more siRNAs comprises a first siRNA targeting a first mutant form of KRAS, and a second siRNA targeting a second mutant form of KRAS. Exemplary two or more siRNAs are not limited but include a) siRNAs targeting both KRAS G12C and KRAS G12D; b) siRNAs targeting both KRAS G12C and KRAS Q61K; c) siRNAs targeting both KRAS G12D and KRAS Q61K; and d) siRNAs targeting KRAS G12C, KRAS G12D and KRAS Q61K. In one preferred embodiment, the first siRNA targets KRAS G12C, and the second siRNA targets KRAS G12D. In some embodiments, the individual has an aberration in a gene encoding the tumor suppressor protein. In some embodiments, the individual has an aberration in the oncogene. In some embodiments, the individual has an aberration in a gene encoding the tumor suppressor protein and the oncogene. In some embodiments, the mRNA encoding the tumor suppressor protein and the siRNA targeting the oncogene are delivered simultaneously. In some embodiments, the mRNA and the siRNA are delivered concurrently. In some embodiments, the mRNA and/or the siRNA are delivered intravenously. In some embodiments, the mRNA and/or the siRNA are complexed with a cell-penetrating peptide when delivered into the individual. In some embodiments, the cell-penetrating peptide is selected from the group consisting of CADY, PEP-1, PEP-2, MPG, VEPEP-3 peptides (used herein interchangeably with ADGN-103 peptides), VEPEP-4 peptides (used herein interchangeably with ADGN-104 peptides), VEPEP-5 peptides (used herein interchangeably with ADGN-105 peptides), VEPEP-6 peptides (used herein interchangeably with ADGN-106 peptides), VEPEP-9 peptides (used herein interchangeably with ADGN-109 peptides), and ADGN-100 peptides. In some embodiments, the cell-penetrating peptide is selected from ADGN 106 peptides and ADGN-100 peptides. In some embodiments, the mRNA (e.g., the mRNA encoding PTEN) and/or the siRNA (e.g., the siRNA targeting KRAS) are delivered about once a week or once every five days. In some embodiments, the dose of the mRNA (e.g., the mRNA encoding PTEN) for each administration in an individual is about 0.001 mg/kg to about 10 mg/kg (e.g., about 0.01 mg/kg to about 1 mg/kg, about 0.02 mg/kg to about 0.1 mg/kg). In some embodiments, the dose of the siRNA (e.g., the siRNA targeting KRAS) for each administration in an individual is about 0.001 mg/kg to about 10 mg/kg (e.g., about 0.01 mg/kg to about 1 mg/kg, about 0.02 mg/kg to about 0.1 mg/kg). In some embodiments, the dose of the mRNA (e.g., the mRNA encoding PTEN) for each administration in an individual is about 0.03 mg/m² to about 400 mg/m² (e.g., about 0.4 mg/m² to about 40 mg/m², about 0.8 mg/m² to about 4 mg/m²). In some embodiments, the dose of the siRNA (e.g., the siRNA targeting KRAS) for each administration in an individual is about 0.03 mg/m² to about 400 mg/m² (e.g., about 0.4 mg/m² to about 40 mg/m², about 0.8 mg/m² to about 4 mg/m²). In some embodiments, the individual is a human. In some embodiments, the mRNA comprises a sequence selected from the group consisting of sequences with accession number of BC005821, JF268690, U92436, CR450306, AK024986, AK313581, U96180, and U93051 and NM_000314 in NCBI GenBank.

In some embodiments, there is provided a method of treating a cancer in an individual, comprising delivering to the individual a) an mRNA encoding a tumor suppressor protein and b) an siRNA targeting an oncogene, wherein the tumor suppressor protein is PTEN, and wherein the oncogene is KRAS. In some embodiments, there is provided a method of treating a cancer in an individual, comprising delivering to the individual a) an mRNA encoding a tumor suppressor protein and b) an siRNA targeting an oncogene, wherein the tumor suppressor protein is p53, and wherein the oncogene is KRAS. In some embodiments, the cancer is selected from pancreatic cancer, ovarian cancer, prostate cancer, and glioblastoma. In some embodiments, the cancer is pancreatic cancer. In some embodiments, there is provided a method of treating pancreatic cancer in an individual, comprising delivering to the individual a) an mRNA encoding a tumor suppressor protein and b) an siRNA targeting an oncogene, wherein the tumor suppressor protein is PTEN, and wherein the oncogene is KRAS. In some embodiments, there is provided a method of treating pancreatic cancer in an individual, comprising delivering to the individual a) an mRNA encoding a tumor suppressor protein and b) an siRNA targeting an oncogene, wherein the tumor suppressor protein is p53, and wherein the oncogene is KRAS. In some embodiments, the individual has an aberration in a gene encoding the tumor suppressor protein. In some embodiments, the individual has an aberration in the oncogene. In some embodiments, the individual has an aberration in a gene encoding the tumor suppressor protein and the oncogene. In some embodiments, the siRNA comprises a cocktail of siRNAs comprising two or more siRNAs. In some embodiments, the two or more siRNAs comprises a first siRNA targeting a first mutant form of KRAS, and a second siRNA targeting a second mutant form of KRAS. Exemplary two or more siRNAs are not limited but include a) siRNAs targeting both KRAS G12C and KRAS G12D; b) siRNAs targeting both KRAS G12C and KRAS Q61K; c) siRNAs targeting both KRAS G12D and KRAS Q61K; and d) siRNAs targeting KRAS G2C, KRAS G12D and KRAS Q61K. In one preferred embodiment, the first siRNA targets KRAS G12C, and the second siRNA targets KRAS G2D. In some embodiments, the mRNA encoding the tumor suppressor protein and the siRNA targeting the oncogene are delivered simultaneously. In some embodiments, the mRNA and the siRNA are delivered concurrently. In some embodiments, the mRNA and/or the siRNA are delivered intravenously. In some embodiments, the mRNA and/or the siRNA are complexed with a cell-penetrating peptide when delivered into the individual. In some embodiments, the cell-penetrating peptide is selected from the group consisting of CADY, PEP-1, PEP-2, MPG, VEPEP-3 peptides (used herein interchangeably with ADGN-103 peptides), VEPEP-4 peptides (used herein interchangeably with ADGN-104 peptides), VEPEP-5 peptides (used herein interchangeably with ADGN-105 peptides), VEPEP-6 peptides (used herein interchangeably with ADGN-106 peptides), VEPEP-9 peptides (used herein interchangeably with ADGN-109 peptides), and ADGN-100 peptides. In some embodiments, the cell-penetrating peptide is selected from ADGN 106 peptides and ADGN-100 peptides. In some embodiments, the mRNA (e.g., the mRNA encoding PTEN) and/or the siRNA (e.g., the siRNA targeting KRAS) are delivered about once a week or once every five days. In some embodiments, the dose of the mRNA (e.g, the mRNA encoding PTEN) for each administration in an individual is about 0.001 mg/kg to about 10 mg/kg (e.g., about 0.01 mg/kg to about 1 mg/kg, about 0.02 mg/kg to about 0.1 mg/kg). In some embodiments, the dose of the siRNA (e.g., the siRNA targeting KRAS) for each administration in an individual is about 0.001 mg/kg to about 10 mg/kg (e.g., about 0.01 mg/kg to about 1 mg/kg, about 0.02 mg/kg to about 0.1 mg/kg). In some embodiments, the dose of the mRNA (e.g., the mRNA encoding PTEN) for each administration in an individual is about 0.03 mg/m² to about 400 mg/m² (e.g., about 0.4 mg/m² to about 40 mg/m², about 0.8 mg/m² to about 4 mg/m²). In some embodiments, the dose of the siRNA (e.g., the siRNA targeting KRAS) for each administration in an individual is about 0.03 mg/m² to about 400 mg/m² (e.g., about 0.4 mg/m² to about 40 mg/m², about 0.8 mg/m² to about 4 mg/m²). In some embodiments, the individual is a human.

In some embodiments, there is provided a method of inhibiting metastasis of a cancer in an individual, comprising delivering to the individual a) an mRNA encoding a tumor suppressor protein and b) an siRNA targeting an oncogene. In some embodiments, the cancer is selected from pancreatic cancer, ovarian cancer, prostate cancer, and glioblastoma. In some embodiments, the cancer is pancreatic cancer. In some embodiments, the tumor suppressor protein is PTEN. In some embodiments, the oncogene is KRAS. In some embodiments, the individual has an aberration in a gene encoding the tumor suppressor protein. In some embodiments, the individual has an aberration in the oncogene. In some embodiments, the individual has an aberration in a gene encoding the tumor suppressor protein and the oncogene. In some embodiments, the siRNA comprises a cocktail of siRNAs comprising two or more siRNAs. In some embodiments, the two or more siRNAs comprises a first siRNA targeting a first mutant form of KRAS, and a second siRNA targeting a second mutant form of KRAS. Exemplary two or more siRNAs are not limited but include a) siRNAs targeting both KRAS G12C and KRAS G12D; b) siRNAs targeting both KRAS G12C and KRAS Q61K; c) siRNAs targeting both KRAS G12D and KRAS Q61K; and d) siRNAs targeting KRAS G12C, KRAS G12D and KRAS Q61K. In one preferred embodiment, the first siRNA targets KRAS G12C, and the second siRNA targets KRAS G12D. In some embodiments, the mRNA encoding the tumor suppressor protein and the siRNA targeting the oncogene are delivered simultaneously. In some embodiments, the mRNA and the siRNA are delivered concurrently. In some embodiments, the mRNA and/or the siRNA are delivered intravenously. In some embodiments, the mRNA and/or the siRNA are complexed with a cell-penetrating peptide when delivered into the individual. In some embodiments, the cell-penetrating peptide is selected from the group consisting of CADY, PEP-1, PEP-2, MPG, VEPEP-3 peptides (used herein interchangeably with ADGN-103 peptides), VEPEP-4 peptides (used herein interchangeably with ADGN-104 peptides), VEPEP-5 peptides (used herein interchangeably with ADGN-105 peptides), VEPEP-6 peptides (used herein interchangeably with ADGN-106 peptides), VEPEP-9 peptides (used herein interchangeably with ADGN-109 peptides), and ADGN-100 peptides. In some embodiments, the cell-penetrating peptide is selected from ADGN 106 peptides and ADGN-100 peptides. In some embodiments, the mRNA (e.g., the mRNA encoding PTEN) and/or the siRNA (e.g., the siRNA targeting KRAS) are delivered about once a week or once every five days. In some embodiments, the dose of the mRNA (e.g., the mRNA encoding PTEN) for each administration in an individual is about 0.001 mg/kg to about 10 mg/kg (e.g., about 0.01 mg/kg to about 1 mg/kg, about 0.02 mg/kg to about 0.1 mg/kg). In some embodiments, the dose of the siRNA (e.g., the siRNA targeting KRAS) for each administration in an individual is about 0.001 mg/kg to about 10 mg/kg (e.g., about 0.01 mg/kg to about 1 mg/kg, about 0.02 mg/kg to about 0.1 mg/kg). In some embodiments, the dose of the mRNA (e.g., the mRNA encoding PTEN) for each administration in an individual is about 0.03 mg/m² to about 400 mg/m² (e.g., about 0.4 mg/m² to about 40 mg/m², about 0.8 mg/m² to about 4 mg/m²). In some embodiments, the dose of the siRNA (e.g., the siRNA targeting KRAS) for each administration in an individual is about 0.03 mg/m² to about 400 mg/m² (e.g., about 0.4 mg/m² to about 40 mg/m², about 0.8 mg/m² to about 4 mg/m²). In some embodiments, the individual is a human.

In some embodiments, there is provided a method of inhibiting metastasis of a cancer in an individual, comprising delivering to the individual a) an mRNA encoding a tumor suppressor protein and b) an siRNA targeting an oncogene, wherein the tumor suppressor protein is PTEN. In some embodiments, there is provided a method of inhibiting metastasis of a cancer in an individual, comprising delivering to the individual a) an mRNA encoding a tumor suppressor protein and b) an siRNA targeting an oncogene, wherein the oncogene is KRAS. In some embodiments, there is provided a method of inhibiting metastasis of a cancer in an individual, comprising delivering to the individual a) an mRNA encoding a tumor suppressor protein and b) an siRNA targeting an oncogene, wherein the tumor suppressor protein is PTEN, and wherein the oncogene is KRAS. In some embodiments, the cancer is selected from pancreatic cancer, ovarian cancer, prostate cancer, and glioblastoma. In some embodiments, the cancer is pancreatic cancer. In some embodiments, the oncogene is KRAS. In some embodiments, the individual has an aberration in a gene encoding the tumor suppressor protein. In some embodiments, the individual has an aberration in the oncogene. In some embodiments, the individual has an aberration in a gene encoding the tumor suppressor protein and the oncogene. In some embodiments, the siRNA comprises a cocktail of siRNAs comprising two or more siRNAs. In some embodiments, the two or more siRNAs comprises a first siRNA targeting a first mutant form of KRAS, and a second siRNA targeting a second mutant form of KRAS. Exemplary two or more siRNAs are not limited but include a) siRNAs targeting both KRAS G12C and KRAS G12D; b) siRNAs targeting both KRAS G12C and KRAS Q61K; c) siRNAs targeting both KRAS G12D and KRAS Q61K; and d) siRNAs targeting KRAS G12C, KRAS G12D and KRAS Q61K. In one preferred embodiment, the first siRNA targets KRAS G12C and the second siRNA targets KRAS G12D. In some embodiments, the mRNA encoding the tumor suppressor protein and the siRNA targeting the oncogene are delivered simultaneously. In some embodiments, the mRNA and the siRNA are delivered concurrently. In some embodiments, the mRNA and/or the siRNA are delivered intravenously. In some embodiments, the mRNA and/or the siRNA are complexed with a cell-penetrating peptide when delivered into the individual. In some embodiments, the cell-penetrating peptide is selected from the group consisting of CADY, PEP-1, PEP-2, MPG, VEPEP-3 peptides (used herein interchangeably with ADGN-103 peptides), VEPEP-4 peptides (used herein interchangeably with ADGN-104 peptides), VEPEP-5 peptides (used herein interchangeably with ADGN-105 peptides), VEPEP-6 peptides (used herein interchangeably with ADGN-106 peptides), VEPEP-9 peptides (used herein interchangeably with ADGN-109 peptides), and ADGN-100 peptides. In some embodiments, the cell-penetrating peptide is selected from ADGN 106 peptides and ADGN-100 peptides. In some embodiments, the mRNA (e.g., the mRNA encoding PTEN) and/or the siRNA (e.g., the siRNA targeting KRAS) are delivered about once a week or once every five days. In some embodiments, the dose of the mRNA (e.g., the mRNA encoding PTEN) for each administration in an individual is about 0.001 mg/kg to about 10 mg/kg (e.g., about 0.01 mg/kg to about 1 mg/kg, about 0.02 mg/kg to about 0.1 mg/kg). In some embodiments, the dose of the siRNA (e.g., the siRNA targeting KRAS) for each administration in an individual is about 0.001 mg/kg to about 10 mg/kg (e.g., about 0.01 mg/kg to about 1 mg/kg, about 0.02 mg/kg to about 0.1 mg/kg). In some embodiments, the dose of the mRNA (e.g., the mRNA encoding PTEN) for each administration in an individual is about 0.03 mg/m² to about 400 mg/m² (e.g., about 0.4 mg/m² to about 40 mg/m², about 0.8 mg/m² to about 4 mg/m²). In some embodiments, the dose of the siRNA (e.g., the siRNA targeting KRAS) for each administration in an individual is about 0.03 mg/m² to about 400 mg/m² (e.g., about 0.4 mg/m² to about 40 mg/m², about 0.8 mg/m² to about 4 mg/m²). In some embodiments, the individual is a human.

In some embodiments, there is provided a method of inhibiting metastasis of pancreatic cancer in an individual, comprising delivering to the individual a) an mRNA encoding a tumor suppressor protein and b) an siRNA targeting an oncogene, wherein the tumor suppressor protein is PTEN, and wherein the oncogene is KRAS. In some embodiments, the individual has an aberration in a gene encoding the tumor suppressor protein. In some embodiments, the individual has an aberration in the oncogene. In some embodiments, the individual has an aberration in a gene encoding the tumor suppressor protein and the oncogene. In some embodiments, the siRNA comprises a cocktail of siRNAs comprising two or more siRNAs. In some embodiments, the two or more siRNAs comprises a first siRNA targeting a first mutant form of KRAS, and a second siRNA targeting a second mutant form of KRAS. Exemplary two or more siRNAs are not limited but include a) siRNAs targeting both KRAS G2C and KRAS G12D; b) siRNAs targeting both KRAS G12C and KRAS Q61K; c) siRNAs targeting both KRAS G12D and KRAS Q61K; and d) siRNAs targeting KRAS G12C. KRAS G12D and KRAS Q61K. In one preferred embodiment, the first siRNA targets KRAS G12C, and the second siRNA targets KRAS G12D. In some embodiments, the mRNA encoding the tumor suppressor protein and the siRNA targeting the oncogene are delivered simultaneously. In some embodiments, the mRNA and the siRNA are delivered concurrently. In some embodiments, the mRNA and/or the siRNA are delivered intravenously. In some embodiments, the mRNA and/or the siRNA are complexed with a cell-penetrating peptide when delivered into the individual. In some embodiments, the cell-penetrating peptide is selected from the group consisting of CADY, PEP-1, PEP-2, MPG, VEPEP-3 peptides (used herein interchangeably with ADGN-103 peptides), VEPEP-4 peptides (used herein interchangeably with ADGN-104 peptides), VEPEP-5 peptides (used herein interchangeably with ADGN-105 peptides), VEPEP-6 peptides (used herein interchangeably with ADGN-106 peptides), VEPEP-9 peptides (used herein interchangeably with ADGN-109 peptides), and ADGN-100 peptides. In some embodiments, the cell-penetrating peptide is selected from ADGN 106 peptides and ADGN-100 peptides. In some embodiments, the mRNA (e.g., the mRNA encoding PTEN) and/or the siRNA (e.g., the siRNA targeting KRAS) are delivered about once a week or once every five days. In some embodiments, the dose of the mRNA (e.g., the mRNA encoding PTEN) for each administration in an individual is about 0.001 mg/kg to about 10 mg/kg (e.g., about 0.01 mg/kg to about 1 mg/kg, about 0.02 mg/kg to about 0.1 mg/kg). In some embodiments, the dose of the siRNA (e.g., the siRNA targeting KRAS) for each administration in an individual is about 0.001 mg/kg to about 10 mg/kg (e.g., about 0.01 mg/kg to about 1 mg/kg, about 0.02 mg/kg to about 0.1 mg/kg). In some embodiments, the dose of the mRNA (e.g., the mRNA encoding PTEN) for each administration in an individual is about 0.03 mg/m² to about 400 mg/m² (e.g., about 0.4 mg/m² to about 40 mg/m², about 0.8 mg/m² to about 4 mg/m²). In some embodiments, the dose of the siRNA (e.g., the siRNA targeting KRAS) for each administration in an individual is about 0.03 mg/m² to about 400 mg/m² (e.g., about 0.4 mg/m² to about 40 mg/m², about 0.8 mg/m² to about 4 mg/m²). In some embodiments, the individual is a human.

In some embodiments, there is provided a method of treating pancreatic cancer in an individual, comprising delivering to the individual a) an mRNA encoding a tumor suppressor protein PTEN and b) an siRNA targeting an oncogene, wherein the mRNA comprises a sequence selected from the group consisting of sequences with accession number of BC005821, JF268690, U92436, CR450306, AK024986, AK313581, U96180, and U93051 and NM_000314 in NCBI GenBank. In some embodiments, the individual has an aberration in a gene encoding the tumor suppressor protein. In some embodiments, the individual has an aberration in the oncogene. In some embodiments, the individual has an aberration in a gene encoding the tumor suppressor protein and the oncogene. In some embodiments, the siRNA comprises a cocktail of siRNAs comprising two or more siRNAs. In some embodiments, the two or more siRNAs comprises a first siRNA targeting a first mutant form of KRAS, and a second siRNA targeting a second mutant form of KRAS. Exemplary two or more siRNAs are not limited but include a) siRNAs targeting both KRAS G12C and KRAS G12D; b) siRNAs targeting both KRAS G12C and KRAS Q61K; c) siRNAs targeting both KRAS G12D and KRAS Q61K; and d) siRNAs targeting KRAS G12C, KRAS G12D and KRAS Q61K. In one preferred embodiment, the first siRNA targets KRAS G12C, and the second siRNA targets KRAS G12D. In some embodiments, the mRNA encoding the tumor suppressor protein and the siRNA targeting the oncogene are delivered simultaneously. In some embodiments, the mRNA and the siRNA are delivered concurrently. In some embodiments, the mRNA and/or the siRNA are delivered intravenously. In some embodiments, the mRNA and/or the siRNA are complexed with a cell-penetrating peptide when delivered into the individual. In some embodiments, the cell-penetrating peptide is selected from the group consisting of CADY, PEP-1, PEP-2, MPG, VEPEP-3 peptides (used herein interchangeably with ADGN-103 peptides), VEPEP-4 peptides (used herein interchangeably with ADGN-104 peptides), VEPEP-5 peptides (used herein interchangeably with ADGN-105 peptides), VEPEP-6 peptides (used herein interchangeably with ADGN-106 peptides), VEPEP-9 peptides (used herein interchangeably with ADGN-109 peptides), and ADGN-100 peptides. In some embodiments, the cell-penetrating peptide is selected from ADGN 106 peptides and ADGN-100 peptides. In some embodiments, the mRNA and/or the siRNA (e.g., the siRNA targeting KRAS) are delivered about once a week or once every five days. In some embodiments, the dose of the mRNA for each administration in an individual is about 0.001 mg/kg to about 10 mg/kg (e.g., about 0.01 mg/kg to about 1 mg/kg, about 0.02 mg/kg to about 0.1 mg/kg). In some embodiments, the dose of the siRNA (e.g., the siRNA targeting KRAS) for each administration in an individual is about 0.001 mg/kg to about 10 mg/kg (e.g., about 0.01 mg/kg to about 1 mg/kg, about 0.02 mg/kg to about 0.1 mg/kg). In some embodiments, the dose of the mRNA for each administration in an individual is about 0.03 mg/m² to about 400 mg/m² (e.g., about 0.4 mg/m² to about 40 mg/m², about 0.8 mg/m² to about 4 mg/m²). In some embodiments, the dose of the siRNA (e.g., the siRNA targeting KRAS) for each administration in an individual is about 0.03 mg/m² to about 400 mg/m² (e.g., about 0.4 mg/m² to about 40 mg/m², about 0.8 mg/m² to about 4 mg/m²). In some embodiments, the individual is a human.

In some embodiments, there is provided a method of treating pancreatic cancer in an individual, comprising delivering to the individual a) an mRNA encoding a tumor suppressor protein p53 and b) an siRNA targeting an oncogene, wherein the mRNA comprises a sequence selected from the group consisting of sequences with accession number of AF052180, NM_000546, AY429684, BT019622, AK223026, DQ186652, DQ186651. DQ186650, DQ186649, DQ186648, DQ263704, DQ286964, DQ191317, DQ401704, AF307851, AM076972, AM076971, AM076970, DQ485152, BC003596, DQ648887, DQ648886, DQ648885, DQ648884, AK225838, M14694, M14695, EF101869, EF101868, EF101867, X01405, AK312568, NM_001126117, NM_001126116, NM_001126115, NM_001126114, NM_001126113, NM_001126112, FJ207420, X60020, X60019, X60018, X60017, X60016, X60015, X60014, X60013, X60011, X60012, X60010, X02469, S66666, AB082923, NM_001126118, JN900492, NM_001276699, NM_001276698, NM_001276697, NM_001276761, NM_001276760, NM_001276696, and NM_001276695 in NCBI GenBank.

In some embodiments, there is provided a method of treating pancreatic cancer in an individual, comprising delivering to the individual a) an mRNA encoding a tumor suppressor protein BRCA1 and b) an siRNA targeting an oncogene, wherein the mRNA comprises a sequence selected from the group consisting of a sequence with accession number of NM_007294, NM_007297, NM_007298, NM 007304, NM 007299, NM_007300, BC046142, BC062429, BC072418, AY354539, AY751490, BC085615, BC106746, BC106745, BC114511, BC115037, U14680, AK293762, U68041, BC030969, BC012577, AK316200, DQ363751, DQ333387, DQ333386, Y08864, JN686490, AB621825, BC038947, U64805, and AF005068 in NCBI GenBank.

In some embodiments, there is provided a method of treating pancreatic cancer in an individual, comprising delivering to the individual a) an mRNA encoding a tumor suppressor protein BRCA2 and b) an siRNA targeting an oncogene, wherein the mRNA comprises a sequence selected from the group consisting of a sequence with accession number of BC047568, NM_000059, DQ897648, BC026160 in NCBI GenBank.

In some embodiments, there is provided a method of treating pancreatic cancer in an individual, comprising delivering to the individual a) an mRNA encoding a tumor suppressor protein TSC1 and b) an siRNA targeting an oncogene, wherein the mRNA comprises a sequence selected from the group consisting of a sequence with accession number of BC047772, NM_000368, BC070032, AB190910, BC108668, BC121000, NM_001162427, NM_001162426, D87683, and AF013168 in NCBI GenBank.

In some embodiments, there is provided a method of treating pancreatic cancer in an individual, comprising delivering to the individual a) an mRNA encoding a tumor suppressor protein TSC2 and b) an siRNA targeting an oncogene, wherein the mRNA comprises a sequence selected from the group consisting of a sequence with accession number of BC046929, BX647816, AK125096, NM_000548, AB210000, NM_001077183, BC150300, BC025364, NM_001114382, AK094152, AK299343, AK295728, AK295672, AK294548, and X75621 in NCBI GenBank.

In some embodiments, there is provided a method of treating pancreatic cancer in an individual, comprising delivering to the individual a) an mRNA encoding a tumor suppressor protein Retinoblastoma 1 (RB1) and b) an siRNA targeting an oncogene, wherein the mRNA comprises a sequence selected from the group consisting of a sequence with accession number of NM_000321, AY429568, AB208788, M19701, AK291258, L41870, AK307730, AK307125, AK300284, AK299179, M33647, M15400, M28419, BC039060, BC040540, and AF043224 in NCBI GenBank.

In some embodiments, there is provided a method of treating pancreatic cancer in an individual, comprising delivering to the individual a) an mRNA encoding a tumor suppressor protein and b) an siRNA targeting an oncogene, wherein the siRNA targets a mutant form of KRAS. In some embodiments, the siRNA specifically targets a mutant form of KRAS but not the wildtype form of KRAS. In some embodiments, the mutant form comprises an aberration of KRAS, wherein the aberration of KRAS comprises a mutation on codon 12, 13, 17, 34 or 61 of KRAS. In some embodiments, the mutant form comprises an aberration of KRAS, wherein the aberration of KRAS is selected from the group consisting of G12C, G12S, G12R, G12F, G12L, G12N, G12A, G12D, G12S, G12V, G13C, G13S, G13R, G13A, G13D, G13V, G13P, S17G, P34S, Q61E, Q61K, Q61L, Q61R, Q61P, Q61H, K117N, A146P, A146T and A146V. In some embodiments, the mutant form comprises an aberration of KRAS, wherein the aberration of KRAS is selected from the group consisting of G12C, G12S, G12R, G12F, G12L, G12N, G12A, G12D, G12V, G13C, G13S, G13D, G13V, G13P, S17G, P34S, Q61K. Q61L, Q61R, and Q61H. In some embodiments, the mutant form comprises an aberration of KRAS, wherein the aberration of KRAS is selected from the group consisting of G12C, G12R, G12S, G12A, G12D, G12V, G13C, G13R, G13S, G13A, G13D, G13V, Q61K, Q61L, Q61R, Q61H, K117N, A146P, A146T and A146V. In some embodiments, the mutant form comprises an aberration of KRAS, wherein the aberration of KRAS is selected from the group consisting of KRAS G12A, G12C, G12D, G12R, G12S, G12V, G13A, G13C, G3D, G13R, G13S, G13V, Q61E, Q61H, Q61K, Q61L, Q61P. and Q61R. In some embodiments, the aberration of KRAS is selected from the group consisting of KRAS G12C, G12D, G12R, G12S, G12V and G13D. In some embodiments, the aberration of KRAS comprises G12C. In some embodiments, the aberration of KRAS comprises G12D. In some embodiments, the aberration of KRAS comprises Q61K. In some embodiments, the aberration of KRAS comprises G12C and G12D. In some embodiments, the aberration of KRAS comprises G12C and Q61K. In some embodiments, the aberration of KRAS comprises G12D and Q61K. In some embodiments, the aberration of KRAS comprises G12C, G12D and Q61K. In some embodiments, the RNAi (e.g., siRNA) comprises one or more sequences of 5′-GUUGGAGCUUGUGGCGUAGTT-3′ (sense) (SEQ ID NO: 83), 5′-CUACGCCACCAGCUCCAACTT-3 (anti-sense) (SEQ ID NO: 84), 5′-GAAGUGCAUACACCGAGACTT-3′ (sense) (SEQ ID NO: 86), 5′-GUCUCGGUGUAGCACUUCT-3′ (anti-sense) (SEQ ID NO: 87), 5′-GUUGGAGCUGUUGGCGUAGTT-3′ (sense) (SEQ ID NO: 88) and/or 5′-CUACGCCAACAGCUCCAACTT-3′ (anti-sense) (SEQ ID NO: 89). In some embodiments, the RNAi (e.g., siRNA) comprises a nucleic acid sequence selected from sequences with SEQ ID NOS: 83, 84, 86-89.

In some embodiments, there is provided a method of treating a cancer in an individual, comprising delivering to the individual a) an mRNA delivery complex comprising an mRNA encoding a tumor suppressor gene (e.g., an mRNA encoding PTEN or p53) complexed with a first cell-penetrating peptide; and b) an RNAi delivery complex comprising an RNAi (e.g., an siRNA targeting an oncogene such as a mutant form of KRAS) complexed with a second cell-penetrating peptide. In some embodiments, the individual has an aberration in a gene encoding the tumor suppressor protein. In some embodiments, the individual has an aberration in the oncogene. In some embodiments, the individual has an aberration in a gene encoding the tumor suppressor protein and the oncogene. In some embodiments, the siRNA comprises a cocktail of siRNAs comprising two or more siRNAs. In some embodiments, the two or more siRNAs comprises a first siRNA targeting a first mutant form of KRAS, and a second siRNA targeting a second mutant form of KRAS. Exemplary two or more siRNAs are not limited but include a) siRNAs targeting both KRAS G12C and KRAS G12D; b) siRNAs targeting both KRAS G12C and KRAS Q61K; c) siRNAs targeting both KRAS G12D and KRAS Q61K; and d) siRNAs targeting KRAS G12C, KRAS G12D and KRAS Q61K. In one preferred embodiment, the first siRNA targets KRAS G12C, and the second siRNA targets KRAS G12D. In some embodiments, the mRNA encoding the tumor suppressor protein and the siRNA targeting the oncogene are delivered simultaneously. In some embodiments, the mRNA and the siRNA are delivered concurrently. In some embodiments, the mRNA and/or the siRNA are delivered intravenously. In some embodiments, the first and/or second cell-penetrating peptide is selected from the group consisting of CADY, PEP-1, PEP-2, MPG, VEPEP-3 peptides (used herein interchangeably with ADGN-103 peptides), VEPEP-4 peptides (used herein interchangeably with ADGN-104 peptides), VEPEP-5 peptides (used herein interchangeably with ADGN-105 peptides), VEPEP-6 peptides (used herein interchangeably with ADGN-106 peptides), VEPEP-9 peptides (used herein interchangeably with ADGN-109 peptides), and ADGN-100 peptides. In some embodiments, the first and/or second cell-penetrating peptide is selected from ADGN 106 peptides and ADGN-100 peptides. In some embodiments, the mRNAand/or the siRNA (e.g., the siRNA targeting KRAS) are delivered about once a week or once every five days. In some embodiments, the dose of the mRNA for each administration in an individual is about 0.001 mg/kg to about 10 mg/kg (e.g., about 0.01 mg/kg to about 1 mg/kg, about 0.02 mg/kg to about 0.1 mg/kg). In some embodiments, the dose of the siRNA (e.g., the siRNA targeting KRAS) for each administration in an individual is about 0.001 mg/kg to about 10 mg/kg (e.g., about 0.01 mg/kg to about 1 mg/kg, about 0.02 mg/kg to about 0.1 mg/kg). In some embodiments, the dose of the mRNA for each administration in an individual is about 0.03 mg/m² to about 400 mg/m² (e.g., about 0.4 mg/m² to about 40 mg/m², about 0.8 mg/m² to about 4 mg/m²). In some embodiments, the dose of the siRNA (e.g., the siRNA targeting KRAS) for each administration in an individual is about 0.03 mg/m² to about 400 mg/m² (e.g., about 0.4 mg/m² to about 40 mg/m², about 0.8 mg/m² to about 4 mg/m²). In some embodiments, the individual is a human.

B. Combination Therapy of Two or More mRNAs

Also provided herein are combination therapies for treating a cancer (e.g., pancreatic cancer) in an individual comprising delivering to the individual a) a first mRNA (e.g., a first mRNA encoding a first therapeutic protein), and b) a second mRNA (e.g., a second mRNA encoding a second therapeutic protein, e.g., a second tumor suppressor protein). In some embodiments, the first therapeutic protein is a tumor suppressor protein (e.g., PTEN, the protein encoded by the PTEN gene). In some embodiments, the second therapeutic protein is a second tumor suppressor protein (e.g., p53 tumor-suppressor protein). In some embodiments, the individual has an aberration in the first or second tumor suppressor protein. In some embodiments, the first mRNA and/or the second mRNA is complexed with a cell-penetrating peptide when delivered into the individual. In some embodiments, the first mRNA and the second mRNA is complexed with a same cell-penetrating peptide when delivered into the individual. In some embodiments, the first mRNA and the second mRNA is complexed with a different cell-penetrating peptide when delivered into the individual. In some embodiments, the first mRNA and the second mRNA are separately complexed with a cell-penetrating peptide when delivered into the individual. In some embodiments, the first mRNA and/or the second mRNA is modified (e.g., wherein at least one modified nucleoside is 5-methoxyuridine (5moU)).

In some embodiments, there is provided a method of treating a cancer in an individual, comprising delivering to the individual a) a first mRNA encoding a first tumor suppressor protein and b) a second mRNA encoding a second tumor suppressor protein. In some embodiments, the cancer is selected from pancreatic cancer, ovarian cancer, prostate cancer, and glioblastoma. In some embodiments, the cancer is pancreatic cancer. In some embodiments, the first mRNA and/or the second mRNA encode a first tumor suppressor protein and/or a second tumor suppressor protein. In some embodiments, the individual has an aberration in the first and/or second tumor suppressor protein. In some embodiments, the first mRNA and/or the second mRNA are complexed with a cell-penetrating peptide when delivered into the individual. In some embodiments, the cell-penetrating peptide is selected from the group consisting of CADY, PEP-1, PEP-2, MPG, VEPEP-3 peptides (used herein interchangeably with ADGN-103 peptides), VEPEP-4 peptides (used herein interchangeably with ADGN-104 peptides), VEPEP-5 peptides (used herein interchangeably with ADGN-105 peptides), VEPEP-6 peptides (used herein interchangeably with ADGN-106 peptides), VEPEP-9 peptides (used herein interchangeably with ADGN-109 peptides), and ADGN-100 peptides. In some embodiments, the cell-penetrating peptide is selected from ADGN 106 peptides and ADGN-100 peptides. In some embodiments, the cell-penetrating peptide comprises an amino acid sequence of SEQ ID NOs: 15-40 (e.g., SEQ ID NO: 77). In some embodiments, the cell-penetrating peptide comprises an amino acid sequence of SEQ ID NO: 53-70 (e.g., SEQ ID NO 79 or 80). In some embodiments, the first mRNA (e.g., the mRNA encoding PTEN) and/or the second mRNA (e.g., the mRNA encoding TP53) are delivered about once a week or once every five days. In some embodiments, the dose of the first mRNA (e.g., the mRNA encoding PTEN) for each administration in an individual is about 0.001 mg/kg to about 50 mg/kg (e.g., about 0.01 mg/kg to about 10 mg/kg, about 0.1 mg/kg to about 1 mg/kg, about 0.5 mg/kg). In some embodiments, the dose of the second mRNA (e.g., the mRNA encoding TP53) for each administration in an individual is about 0.001 mg/kg to about 50 mg/kg (e.g., about 0.01 mg/kg to about 10 mg/kg, about 0.1 mg/kg to about 1 mg/kg, about 0.5 mg/kg). In some embodiments, the dose of the mRNA (e.g., the mRNA encoding PTEN) for each administration in an individual is about 0.003 mg/m² to about 150 mg/m² (e.g., about 0.03 mg/m² to about 30 mg/m², about 0.3 mg/m² to about 3 mg/m²). In some embodiments, the dose of the second mRNA (e.g., the mRNA encoding TP53) for each administration in an individual is about 0.003 mg/m² to about 150 mg/m² (e.g., about 0.03 mg/m² to about 30 mg/m², about 0.3 mg/m² to about 3 mg/m²). In some embodiments, the individual is a human. In some embodiments, the first mRNA and/or the second mRNA is administered intravenously or subcutaneously.

In some embodiments, there is provided a method of treating a cancer (e.g., a pancreatic cancer in an individual, comprising delivering to the individual a) a first mRNA encoding PTEN and b) a second mRNA encoding p53. In some embodiments, the first mRNA and/or the second mRNA are complexed with a cell-penetrating peptide when delivered into the individual. In some embodiments, the cell-penetrating peptide is selected from the group consisting of CADY, PEP-1, PEP-2, MPG, VEPEP-3 peptides (used herein interchangeably with ADGN-103 peptides), VEPEP-4 peptides (used herein interchangeably with ADGN-104 peptides), VEPEP-5 peptides (used herein interchangeably with ADGN-105 peptides), VEPEP-6 peptides (used herein interchangeably with ADGN-106 peptides), VEPEP-9 peptides (used herein interchangeably with ADGN-109 peptides), and ADGN-100 peptides. In some embodiments, the cell-penetrating peptide is selected from ADGN 106 peptides and ADGN-100 peptides. In some embodiments, the cell-penetrating peptide comprises an amino acid sequence of SEQ ID NOs: 15-40 (e.g., SEQ ID NO: 77). In some embodiments, the cell-penetrating peptide comprises an amino acid sequence of SEQ ID NO: 53-70 (e.g., SEQ ID NO 79 or 80). In some embodiments, the first mRNA and/or the second mRNA are delivered about once a week or once every five days. In some embodiments, the dose of the first mRNA for each administration in an individual is about 0.001 mg/kg to about 50 mg/kg (e.g., about 0.01 mg/kg to about 10 mg/kg, about 0.1 mg/kg to about 1 mg/kg, about 0.5 mg/kg). In some embodiments, the dose of the second mRNA for each administration in an individual is about 0.001 mg/kg to about 50 mg/kg (e.g., about 0.01 mg/kg to about 10 mg/kg, about 0.1 mg/kg to about 1 mg/kg, about 0.5 mg/kg). In some embodiments, the dose of the mRNA for each administration in an individual is about 0.003 mg/m² to about 150 mg/m² (e.g., about 0.03 mg/m² to about 30 mg/m², about 0.3 mg/m² to about 3 mg/m²). In some embodiments, the dose of the second mRNA for each administration in an individual is about 0.003 mg/m² to about 150 mg/m² (e.g., about 0.03 mg/m² to about 30 mg/m², about 0.3 mg/m² to about 3 mg/m²). In some embodiments, the individual is a human. In some embodiments, the first mRNA and or the second mRNA is administered intravenously or subcutaneously.

In some embodiments, there is provided a method of treating a cancer (e.g., a pancreatic cancer) in an individual, comprising delivering to the individual a) a first mRNA delivery complex comprising a first mRNA complexed with a first cell-penetrating peptide, wherein the first mRNA encodes PTEN; and b) a second mRNA delivery complex comprising a second mRNA complexed with a second cell-penentrating peptide, wherein the second mRNA encodes p53. In some embodiments, the cancer is selected from pancreatic cancer, ovarian cancer, prostate cancer, and glioblastoma. In some embodiments, the first and/or the second cell-penetrating peptide is selected from the group consisting of CADY, PEP-1, PEP-2, MPG, VEPEP-3 peptides (used herein interchangeably with ADGN-103 peptides), VEPEP-4 peptides (used herein interchangeably with ADGN-104 peptides), VEPEP-5 peptides (used herein interchangeably with ADGN-105 peptides), VEPEP-6 peptides (used herein interchangeably with ADGN-106 peptides), VEPEP-9 peptides (used herein interchangeably with ADGN-109 peptides), and ADGN-100 peptides. In some embodiments, the first and/or the second cell-penetrating peptide is selected from ADGN 106 peptides and ADGN-100 peptides. In some embodiments, the first and/or the second cell-penetrating peptide comprises an amino acid sequence of SEQ ID NOs: 15-40 (e.g., SEQ ID NO: 77). In some embodiments, the first or the second cell-penetrating peptide comprises an amino acid sequence of SEQ ID NO: 53-70 (e.g., SEQ ID NO 79 or 80). In some embodiments, the first mRNA and/or the second mRNA are delivered about once a week or once every five days. In some embodiments, the dose of the first mRNA for each administration in an individual is about 0.001 mg/kg to about 50 mg/kg (e.g., about 0.01 mg/kg to about 10 mg/kg, about 0.1 mg/kg to about 1 mg/kg, about 0.5 mg/kg). In some embodiments, the dose of the second mRNA for each administration in an individual is about 0.001 mg/kg to about 50 mg/kg (e.g., about 0.01 mg/kg to about 10 mg/kg, about 0.1 mg/kg to about 1 mg/kg, about 0.5 mg/kg). In some embodiments, the dose of the mRNA for each administration in an individual is about 0.003 mg/m² to about 150 mg/m² (e.g., about 0.03 mg/m² to about 30 mg/m², about 0.3 mg/m² to about 3 mg/m²). In some embodiments, the dose of the second mRNA for each administration in an individual is about 0.003 mg/m² to about 150 mg/m² (e.g., about 0.03 mg/m² to about 30 mg/m², about 0.3 mg/m² to about 3 mg/m²). In some embodiments, the individual is a human. In some embodiments, the first mRNA and or the second mRNA is administered intravenously or subcutaneously.

In some embodiments, there is provided a method of treating a cancer (e.g., a pancreatic cancer) in an individual, comprising delivering to the individual an mRNA delivery complex comprising a) a first mRNA, wherein the first mRNA encodes PTEN; b) a second mRNA, wherein the second mRNA encodes p53; and c) a cell-penetrating peptide. In some embodiments, the cancer is selected from pancreatic cancer, ovarian cancer, prostate cancer, and glioblastoma, the cell-penetrating peptide is selected from the group consisting of CADY, PEP-1, PEP-2, MPG, VEPEP-3 peptides (used herein interchangeably with ADGN-103 peptides), VEPEP-4 peptides (used herein interchangeably with ADGN-104 peptides), VEPEP-5 peptides (used herein interchangeably with ADGN-105 peptides), VEPEP-6 peptides (used herein interchangeably with ADGN-106 peptides), VEPEP-9 peptides (used herein interchangeably with ADGN-109 peptides), and ADGN-100 peptides. In some embodiments, the cell-penetrating peptide is selected from ADGN 106 peptides and ADGN-100 peptides. In some embodiments, the cell-penetrating peptide comprises an amino acid sequence of SEQ ID NOs: 15-40 (e.g., SEQ ID NO: 77). In some embodiments, the cell-penetrating peptide comprises an amino acid sequence of SEQ ID NO: 53-70 (e.g., SEQ ID NO 79 or 80). In some embodiments, the first mRNA and/or the second mRNA are delivered about once a week or once every five days. In some embodiments, the dose of the first mRNA for each administration in an individual is about 0.001 mg/kg to about 50 mg/kg (e.g., about 0.01 mg/kg to about 10 mg/kg, about 0.1 mg/kg to about 1 mg/kg, about 0.5 mg/kg). In some embodiments, the dose of the second mRNA for each administration in an individual is about 0.001 mg/kg to about 50 mg/kg (e.g., about 0.01 mg/kg to about 10 mg/kg, about 0.1 mg/kg to about 1 mg/kg, about 0.5 mg/kg). In some embodiments, the dose of the mRNA for each administration in an individual is about 0.003 mg/m² to about 150 mg/m² (e.g., about 0.03 mg/m² to about 30 mg/m², about 0.3 mg/m² to about 3 mg/m). In some embodiments, the dose of the second mRNA for each administration in an individual is about 0.003 mg/m² to about 150 mg/m² (e.g., about 0.03 mg/m² to about 30 mg/m², about 0.3 mg/m² to about 3 mg/m²). In some embodiments, the individual is a human. In some embodiments, the first mRNA and or the second mRNA is administered intravenously or subcutaneously.

C. Combination Therapy of mRNA and/or RNAi (e.g., siRNA) and Another Agent

Also provided herein are combination therapies for treating a cancer (e.g., pancreatic cancer) in an individual comprising delivering to the individual a) an mRNA and/or an RNAi (e.g., an siRNA) described herein, b) a second agent. In some embodiments, the second agent comprises a nanoparticle composition as described herein. In some embodiments, the nanoparticle composition comprising a taxane (e.g., paclitaxel) or an mTOR inhibitor (e.g., rapamycin) and a carrier protein (e.g., albumin, e.g., human serum albumin). In some embodiments, the taxane is paclitaxel. In some embodiments, the other agent is nab-paclitaxel. In some embodiments, the mTOR inhibitor is rapamycin. In some embodiments, the other agent is nab-rapamycin. In some embodiments, the method further comprises administering a chemotherapeutic agent (e.g., gemcitabine). In some embodiments, the mRNA is modified (e.g., wherein at least one modified nucleoside is 5-methoxyuridine (5moU)).

In some embodiments, there is provided a method of treating a cancer in an individual comprising delivering to the individual a) an mRNA encoding a tumor suppressor protein, and b) a nanoparticle composition comprising a taxane (e.g., paclitaxel) or an mTOR inhibitor (e.g., rapamycin) and a carrier protein (e.g., albumin). In some embodiments, the tumor suppressor protein corresponds to a tumor-suppressor gene. In some embodiments, the corresponding tumor-suppressor gene includes, without limitation, PTEN, Retinoblastoma RB (or RB1), TP53, TP63, TP73, CDKN2A (INK4A), CDKN1B, CDKN1C, DLD/NP1, HEPACAM, SDHB, SDHD, SFRP1, TCF21, TIG1, MLH1, MSH2, MSH6, WT1, WT2, NF1, NF2N, VHL, KLF4, pVHL, APC, CD95, ST5, YPEL3, ST7, APC, MADR2, BRCA1, BRCA2, Patched, TSC1, TSC2, PALB2, ST14, or VHL. In some embodiments, the tumor suppressor gene is selected from PB1, TSC1, TSC2, BRCA1, BRCA2, PTEN and TP53. In some embodiments, the cancer is selected from breast cancer, lung cancer, pancreatic cancer, ovarian cancer, prostate cancer, and glioblastoma. In some embodiments, the individual has an aberration in a gene encoding the tumor suppressor protein. In some embodiments, the mRNA is delivered intravenously. In some embodiments, the mRNA is complexed with a cell-penetrating peptide when delivered into the individual. In some embodiments, the cell-penetrating peptide is selected from the group consisting of CADY, PEP-1, PEP-2, MPG, VEPEP-3 peptides (used herein interchangeably with ADGN-103 peptides), VEPEP-4 peptides (used herein interchangeably with ADGN-104 peptides), VEPEP-5 peptides (used herein interchangeably with ADGN-105 peptides). VEPEP-6 peptides (used herein interchangeably with ADGN-106 peptides), VEPEP-9 peptides (used herein interchangeably with ADGN-109 peptides), and ADGN-100 peptides. In some embodiments, the cell-penetrating peptide is selected from ADGN 106 peptides and ADGN-100 peptides. In some embodiments, the mRNA is delivered about once a week or once every five days. In some embodiments, the dose of the mRNA for each administration in an individual is about 0.001 mg/kg to about 10 mg/kg (e.g., about 0.01 mg/kg to about 1 mg/kg, about 0.02 mg/kg to about 0.1 mg/kg). In some embodiments, the dose of the mRNA for each administration in an individual is about 0.03 mg/m² to about 400 mg/m² (e.g., about 0.4 mg/m² to about 40 mg/m², about 0.8 mg/m² to about 4 mg/m²). In some embodiments, the individual is a human. In some embodiments, the nanoparticles comprising a taxane (e.g., paclitaxel) or an mTOR inhibitor (e.g., rapamycin) have an average diameter of no greater than 200 nm. In some embodiments, the taxane (e.g., paclitaxel) or the mTOR inhibitor (e.g., rapamycin) in the nanoparticles are coated with the carrier protein (e.g., albumin). In some embodiments, the weight ratio of the carrier protein (e.g., albumin) and the taxane (e.g., paclitaxel) or the mTOR inhibitor (e.g., rapamycin) in the nanoparticle composition is about 9:1 or less. In some embodiments, the albumin is human albumin. In some embodiments, the dose of the taxane (e.g., paclitaxel) or the mTOR inhibitor (e.g., rapamycin) in the nanoparticle composition is about 1 mg/m2 to about 150 mg/m2. In some embodiments, the dose of the taxane (e.g., paclitaxel) or the mTOR inhibitor (e.g., rapamycin) in the nanoparticle composition is about 10 mg/m2 to about 50 mg/m2.

In some embodiments, there is provided a method of treating a cancer in an individual comprising delivering to the individual a) an RNAi (e.g., an siRNA) targeting an oncogene (e.g., KRAS), and b) a nanoparticle composition comprising a taxane (e.g., paclitaxel) or an mTOR inhibitor (e.g., rapamycin) and a carrier protein (e.g., albumin). In some embodiments, the RNAi (e.g., siRNA) targets a mutant form of KRAS, wherein the mutant form of KRAS comprises a mutation on codon 12 or 61 of KRAS. In some embodiments, the mutant form of KRAS comprises G12D KRAS and/or G12C KRAS. In some embodiments, the individual has an aberration in the oncogene. In some embodiments, the siRNA is complexed with a cell-penetrating peptide when delivered into the individual. In some embodiments, the cell-penetrating peptide is selected from the group consisting of CADY, PEP-1, PEP-2, MPG, VEPEP-3 peptides (used herein interchangeably with ADGN-103 peptides), VEPEP-4 peptides (used herein interchangeably with ADGN-104 peptides), VEPEP-5 peptides (used herein interchangeably with ADGN-105 peptides), VEPEP-6 peptides (used herein interchangeably with ADGN-106 peptides), VEPEP-9 peptides (used herein interchangeably with ADGN-109 peptides), and ADGN-100 peptides. In some embodiments, the cell-penetrating peptide is selected from ADGN 106 peptides and ADGN-100 peptides. In some embodiments, the siRNA (e.g., the siRNA targeting KRAS) are delivered about once a week or once every five days. In some embodiments, the dose of the siRNA (e.g., the siRNA targeting KRAS) for each administration in an individual is about 0.001 mg/kg to about 10 mg/kg (e.g., about 0.01 mg/kg to about 1 mg/kg, about 0.02 mg/kg to about 0.1 mg/kg). In some embodiments, the dose of the siRNA (e.g., the siRNA targeting KRAS) for each administration in an individual is about 0.03 mg/m² to about 400 mg/m² (e.g., about 0.4 mg/m² to about 40 mg/m², about 0.8 mg/m² to about 4 mg/m²). In some embodiments, the individual is a human. In some embodiments, the nanoparticles comprising a taxane (e.g., paclitaxel) or an mTOR inhibitor (e.g., rapamycin) have an average diameter of no greater than 200 nm. In some embodiments, the taxane (e.g., paclitaxel) or the mTOR inhibitor (e.g., rapamycin) in the nanoparticles are coated with the carrier protein (e.g., albumin). In some embodiments, the weight ratio of the carrier protein (e.g., albumin) and the taxane (e.g., paclitaxel) or the mTOR inhibitor (e.g., rapamycin) in the nanoparticle composition is about 9:1 or less. In some embodiments, the albumin is human albumin. In some embodiments, the dose of the taxane (e.g., paclitaxel) or the mTOR inhibitor (e.g., rapamycin) in the nanoparticle composition is about 1 mg/m² to about 150 mg/m². In some embodiments, the dose of the taxane (e.g., paclitaxel) or the mTOR inhibitor (e.g., rapamycin) in the nanoparticle composition is about 10 mg/m2 to about 50 mg/m2.

In some embodiments, there is provided a method of treating a cancer in an individual comprising delivering to the individual a) an mRNA encoding a tumor suppressor protein, b) an RNAi (e.g., an siRNA) targeting an oncogene (e.g., KRAS), and c) a nanoparticle composition comprising a taxane (e.g., paclitaxel) or an mTOR inhibitor (e.g., rapamycin) and a carrier protein (e.g., albumin). In some embodiments, the tumor suppressor protein corresponds to a tumor-suppressor gene. In some embodiments, the corresponding tumor-suppressor gene includes, without limitation, PTEN, Retinoblastoma RB (or RB1), TP53, TP63, TP73, CDKN2A (INK4A), CDKN1B, CDKN1C. DLD/NP1, HEPACAM, SDHB, SDHD, SFRP1, TCF21, TIG1, MLH1, MSH2. MSH6, WT1, WT2, NF1, NF2N, VHL, KLF4, pVHL, APC, CD95, ST5, YPEL3, ST7, APC, MADR2, BRCA1, BRCA2, Patched, TSC1, TSC2, PALB2, ST14, or VHL. In some embodiments, the tumor suppressor gene is selected from PB1, TSC1, TSC2, BRCA1, BRCA2, PTEN and TP53. In some embodiments, the cancer is selected from breast cancer, lung cancer, pancreatic cancer, ovarian cancer, prostate cancer, and glioblastoma. In some embodiments, the RNAi (e.g., siRNA) targets a mutant form of KRAS, wherein the mutant form of KRAS comprises a mutation on codon 12 or 61 of KRAS. In some embodiments, the mutant form of KRAS comprises G12D KRAS and/or G12C KRAS. In some embodiments, the individual has an aberration in a gene encoding the tumor suppressor protein. In some embodiments, the individual has an aberration in the oncogene. In some embodiments, the individual has an aberration in a gene encoding the tumor suppressor protein and the oncogene. In some embodiments, the mRNA encoding the tumor suppressor protein and the siRNA targeting the oncogene are delivered simultaneously. In some embodiments, the mRNA and the siRNA are delivered concurrently. In some embodiments, the mRNA and/or the siRNA are delivered intravenously. In some embodiments, the mRNA and/or the siRNA are complexed with a cell-penetrating peptide when delivered into the individual. In some embodiments, the cell-penetrating peptide is selected from the group consisting of CADY, PEP-1, PEP-2, MPG, VEPEP-3 peptides (used herein interchangeably with ADGN-103 peptides), VEPEP-4 peptides (used herein interchangeably with ADGN-104 peptides), VEPEP-5 peptides (used herein interchangeably with ADGN-105 peptides), VEPEP-6 peptides (used herein interchangeably with ADGN-106 peptides), VEPEP-9 peptides (used herein interchangeably with ADGN-109 peptides), and ADGN-100 peptides. In some embodiments, the cell-penetrating peptide is selected from ADGN 106 peptides and ADGN-100 peptides. In some embodiments, the mRNA (e.g., the mRNA encoding PTEN) and/or the siRNA (e.g., the siRNA targeting KRAS) are delivered about once a week or once every five days. In some embodiments, the dose of the mRNA (e.g., the mRNA encoding PTEN) for each administration in an individual is about 0.001 mg/kg to about 10 mg/kg (e.g., about 0.01 mg/kg to about 1 mg/kg, about 0.02 mg/kg to about 0.1 mg/kg). In some embodiments, the dose of the siRNA (e.g., the siRNA targeting KRAS) for each administration in an individual is about 0.001 mg/kg to about 10 mg/kg (e.g., about 0.01 mg/kg to about 1 mg/kg, about 0.02 mg/kg to about 0.1 mg/kg). In some embodiments, the dose of the mRNA (e.g., the mRNA encoding PTEN) for each administration in an individual is about 0.03 mg/m² to about 40 mg/m² (e.g., about 0.4 mg/m² to about 40 mg/m², about 0.8 mg/m² to about 4 mg/m). In some embodiments, the dose of the siRNA (e.g., the siRNA targeting KRAS) for each administration in an individual is about 0.03 mg/m² to about 400 mg/m² (e.g., about 0.4 mg/m² to about 40 mg/m², about 0.8 mg/m² to about 4 mg/m²). In some embodiments, the individual is a human. In some embodiments, the nanoparticles comprising a taxane (e.g., paclitaxel) or an mTOR inhibitor (e.g., rapamycin) have an average diameter of no greater than 200 nm. In some embodiments, the taxane (e.g., paclitaxel) or the mTOR inhibitor (e.g., rapamycin) in the nanoparticles are coated with the carrier protein (e.g., albumin). In some embodiments, the weight ratio of the carrier protein (e.g., albumin) and the taxane (e.g., paclitaxel) or the mTOR inhibitor (e.g., rapamycin) in the nanoparticle composition is about 9:1 or less. In some embodiments, the albumin is human albumin. In some embodiments, the dose of the taxane (e.g., paclitaxel) or the mTOR inhibitor (e.g., rapamycin) in the nanoparticle composition is about 1 mg/m2 to about 150 mg/m2. In some embodiments, the dose of the taxane (e.g., paclitaxel) or the mTOR inhibitor (e.g., rapamycin) in the nanoparticle composition is about 10 mg/m2 to about 50 mg/m2.

In some embodiments, there is provided a method of treating a cancer (e.g., a pancreatic cancer) in an individual comprising delivering to the individual a) an mRNA encoding a tumor suppressor protein, and b) a nanoparticle composition comprising a taxane (e.g., paclitaxel) or an mTOR inhibitor (e.g., rapamycin) and a carrier protein (e.g., albumin). In some embodiments, the tumor suppressor protein is PTEN or p53. In some embodiments, the individual has an aberration in a gene encoding the tumor suppressor protein. In some embodiments, the mRNA is delivered intravenously. In some embodiments, the mRNA is complexed with a cell-penetrating peptide when delivered into the individual. In some embodiments, the cell-penetrating peptide is selected from the group consisting of CADY, PEP-1, PEP-2, MPG. VEPEP-3 peptides (used herein interchangeably with ADGN-103 peptides), VEPEP-4 peptides (used herein interchangeably with ADGN-104 peptides), VEPEP-5 peptides (used herein interchangeably with ADGN-105 peptides), VEPEP-6 peptides (used herein interchangeably with ADGN-106 peptides), VEPEP-9 peptides (used herein interchangeably with ADGN-109 peptides), and ADGN-100 peptides. In some embodiments, the cell-penetrating peptide is selected from ADGN 106 peptides and ADGN-100 peptides. In some embodiments, the mRNA is delivered about once a week or once every five days. In some embodiments, the dose of the mRNA for each administration in an individual is about 0.001 mg/kg to about 10 mg/kg (e.g., about 0.01 mg/kg to about 1 mg/kg, about 0.02 mg/kg to about 0.1 mg/kg). In some embodiments, the dose of the mRNA for each administration in an individual is about 0.03 mg/m² to about 400 mg/m² (e.g., about 0.4 mg/m² to about 40 mg/m², about 0.8 mg/m² to about 4 mg/m²). In some embodiments, the individual is a human. In some embodiments, the nanoparticles comprising a taxane (e.g., paclitaxel) or an mTOR inhibitor (e.g., rapamycin) have an average diameter of no greater than 200 nm. In some embodiments, the taxane (e.g., paclitaxel) or the mTOR inhibitor (e.g., rapamycin) in the nanoparticles are coated with the carrier protein (e.g., albumin). In some embodiments, the weight ratio of the carrier protein (e.g., albumin) and the taxane (e.g., paclitaxel) or the mTOR inhibitor (e.g., rapamycin) in the nanoparticle composition is about 9:1 or less. In some embodiments, the albumin is human albumin. In some embodiments, the dose of the taxane (e.g., paclitaxel) or the mTOR inhibitor (e.g., rapamycin) in the nanoparticle composition is about 1 mg/m2 to about 150 mg/m2. In some embodiments, the dose of the taxane (e.g., paclitaxel) or the mTOR inhibitor (e.g., rapamycin) in the nanoparticle composition is about 10 mg/m2 to about 50 mg/m2.

In some embodiments, there is provided a method of treating a cancer (e.g., a pancreatic cancer) in an individual comprising delivering to the individual a) an RNAi (e.g., an siRNA) targeting an oncogene (e.g., KRAS), and b) a nanoparticle composition comprising a taxane (e.g., paclitaxel) or an mTOR inhibitor (e.g., rapamycin) and a carrier protein (e.g., albumin). In some embodiments, the RNAi (e.g., siRNA) targets a mutant form of KRAS, wherein the mutant form of KRAS comprises a mutation on codon 12 or 61 of KRAS. In some embodiments, the mutant form of KRAS comprises G12D KRAS and/or G12C KRAS. In some embodiments, the individual has an aberration in the oncogene. In some embodiments, the siRNA is complexed with a cell-penetrating peptide when delivered into the individual. In some embodiments, the cell-penetrating peptide is selected from the group consisting of CADY. PEP-1, PEP-2, MPG, VEPEP-3 peptides (used herein interchangeably with ADGN-103 peptides), VEPEP-4 peptides (used herein interchangeably with ADGN-104 peptides), VEPEP-5 peptides (used herein interchangeably with ADGN-105 peptides), VEPEP-6 peptides (used herein interchangeably with ADGN-106 peptides), VEPEP-9 peptides (used herein interchangeably with ADGN-109 peptides), and ADGN-100 peptides. In some embodiments, the cell-penetrating peptide is selected from ADGN 106 peptides and ADGN-100 peptides. In some embodiments, the siRNA (e.g., the siRNA targeting KRAS) are delivered about once a week or once every five days. In some embodiments, the dose of the siRNA (e.g., the siRNA targeting KRAS) for each administration in an individual is about 0.001 mg/kg to about 10 mg/kg (e.g., about 0.01 mg/kg to about 1 mg/kg, about 0.02 mg/kg to about 0.1 mg/kg). In some embodiments, the dose of the siRNA (e.g., the siRNA targeting KRAS) for each administration in an individual is about 0.03 mg/m² to about 400 mg/m² (e.g., about 0.4 mg/m² to about 40 mg/m², about 0.8 mg/m² to about 4 mg/m²). In some embodiments, the individual is a human. In some embodiments, the nanoparticles comprising a taxane (e.g., paclitaxel) or an mTOR inhibitor (e.g., rapamycin) have an average diameter of no greater than 200 nm. In some embodiments, the taxane (e.g., paclitaxel) or the mTOR inhibitor (e.g., rapamycin) in the nanoparticles are coated with the carrier protein (e.g., albumin). In some embodiments, the weight ratio of the carrier protein (e.g., albumin) and the taxane (e.g., paclitaxel) or the mTOR inhibitor (e.g., rapamycin) in the nanoparticle composition is about 9:1 or less. In some embodiments, the albumin is human albumin. In some embodiments, the dose of the taxane (e.g., paclitaxel) or the mTOR inhibitor (e.g., rapamycin) in the nanoparticle composition is about 1 mg/m2 to about 150 mg/m2. In some embodiments, the dose of the taxane (e.g., paclitaxel) or the mTOR inhibitor (e.g., rapamycin) in the nanoparticle composition is about 10 mg/m2 to about 50 mg/m2.

In some embodiments, there is provided a method of treating a cancer (e.g., a pancreatic cancer) in an individual comprising delivering to the individual a) an mRNA encoding a tumor suppressor protein, b) an siRNA targeting an oncogene (e.g., KRAS), and c) a nanoparticle composition comprising a taxane (e.g., paclitaxel) or an mTOR inhibitor (e.g., rapamycin) and a carrier protein (e.g., albumin). In some embodiments, the tumor suppressor protein is PTEN or p53. In some embodiments, the RNAi (e.g., siRNA) targets a mutant form of KRAS, wherein the mutant form of KRAS comprises a mutation on codon 12 or 61 of KRAS. In some embodiments, the mutant form of KRAS comprises G12D KRAS and/or G12C KRAS. In some embodiments, the individual has an aberration in a gene encoding the tumor suppressor protein. In some embodiments, the individual has an aberration in the oncogene. In some embodiments, the individual has an aberration in a gene encoding the tumor suppressor protein and the oncogene. In some embodiments, the mRNA encoding the tumor suppressor protein and the siRNA targeting the oncogene are delivered simultaneously. In some embodiments, the mRNA and the siRNA are delivered concurrently. In some embodiments, the mRNA and/or the siRNA are delivered intravenously. In some embodiments, the mRNA and/or the siRNA are complexed with a cell-penetrating peptide when delivered into the individual. In some embodiments, the cell-penetrating peptide is selected from the group consisting of CADY, PEP-1, PEP-2, MPG, VEPEP-3 peptides (used herein interchangeably with ADGN-103 peptides), VEPEP-4 peptides (used herein interchangeably with ADGN-104 peptides), VEPEP-5 peptides (used herein interchangeably with ADGN-105 peptides), VEPEP-6 peptides (used herein interchangeably with ADGN-106 peptides), VEPEP-9 peptides (used herein interchangeably with ADGN-109 peptides), and ADGN-100 peptides. In some embodiments, the cell-penetrating peptide is selected from ADGN 106 peptides and ADGN-100 peptides. In some embodiments, the mRNA (e.g., the mRNA encoding PTEN) and/or the siRNA (e.g., the siRNA targeting KRAS) are delivered about once a week or once every five days. In some embodiments, the dose of the mRNA (e.g., the mRNA encoding PTEN) for each administration in an individual is about 0.001 mg/kg to about 10 mg/kg (e.g., about 0.01 mg/kg to about 1 mg/kg, about 0.02 mg/kg to about 0.1 mg/kg). In some embodiments, the dose of the siRNA (e.g., the siRNA targeting KRAS) for each administration in an individual is about 0.001 mg/kg to about 10 mg/kg (e.g., about 0.01 mg/kg to about 1 mg/kg, about 0.02 mg/kg to about 0.1 mg/kg). In some embodiments, the dose of the mRNA (e.g., the mRNA encoding PTEN) for each administration in an individual is about 0.03 mg/m² to about 40 mg/m² (e.g., about 0.4 mg/m² to about 40 mg/m², about 0.8 mg/m² to about 4 mg/m²). In some embodiments, the dose of the siRNA (e.g., the siRNA targeting KRAS) for each administration in an individual is about 0.03 mg/m² to about 400 mg/m² (e.g., about 0.4 mg/m to about 40 mg/m², about 0.8 mg/m² to about 4 mg/m²). In some embodiments, the individual is a human. In some embodiments, the nanoparticles comprising a taxane (e.g., paclitaxel) or an mTOR inhibitor (e.g., rapamycin) have an average diameter of no greater than 200 nm. In some embodiments, the taxane (e.g., paclitaxel) or the mTOR inhibitor (e.g., rapamycin) in the nanoparticles are coated with the carrier protein (e.g., albumin). In some embodiments, the weight ratio of the carrier protein (e.g., albumin) and the taxane (e.g., paclitaxel) or the mTOR inhibitor (e.g., rapamycin) in the nanoparticle composition is about 9:1 or less. In some embodiments, the albumin is human albumin. In some embodiments, the dose of the taxane (e.g., paclitaxel) or the mTOR inhibitor (e.g., rapamycin) in the nanoparticle composition is about 1 mg/m² to about 150 mg/m². In some embodiments, the dose of the taxane (e.g., paclitaxel) or the mTOR inhibitor (e.g., rapamycin) in the nanoparticle composition is about 10 mg/m² to about 50 mg/m².

In some embodiments, there is provided a method of treating a cancer (e.g., a pancreatic cancer) in an individual comprising delivering to the individual a) an mRNA encoding a tumor suppressor protein, b) a nanoparticle composition comprising a taxane (e.g., paclitaxel) or an mTOR inhibitor (e.g., rapamycin) and a carrier protein (e.g., albumin), and c) an effective amount of gemcitabine. In some embodiments, the tumor suppressor protein is PTEN or p53. In some embodiments, the individual has an aberration in a gene encoding the tumor suppressor protein. In some embodiments, the mRNA is delivered intravenously. In some embodiments, the mRNA is complexed with a cell-penetrating peptide when delivered into the individual. In some embodiments, the cell-penetrating peptide is selected from the group consisting of CADY, PEP-1, PEP-2, MPG, VEPEP-3 peptides (used herein interchangeably with ADGN-103 peptides), VEPEP-4 peptides (used herein interchangeably with ADGN-104 peptides), VEPEP-5 peptides (used herein interchangeably with ADGN-105 peptides), VEPEP-6 peptides (used herein interchangeably with ADGN-106 peptides), VEPEP-9 peptides (used herein interchangeably with ADGN-109 peptides), and ADGN-100 peptides. In some embodiments, the cell-penetrating peptide is selected from ADGN 106 peptides and ADGN-100 peptides. In some embodiments, the mRNA is delivered about once a week or once every five days. In some embodiments, the dose of the mRNA for each administration in an individual is about 0.001 mg/kg to about 10 mg/kg (e.g., about 0.01 mg/kg to about 1 mg/kg, about 0.02 mg/kg to about 0.1 mg/kg). In some embodiments, the dose of the mRNA for each administration in an individual is about 0.03 mg/m² to about 400 mg/m² (e.g., about 0.4 mg/m² to about 40 mg/m², about 0.8 mg/m² to about 4 mg/m²). In some embodiments, the individual is a human. In some embodiments, the nanoparticles comprising a taxane (e.g., paclitaxel) or an mTOR inhibitor (e.g., rapamycin) have an average diameter of no greater than 200 nm. In some embodiments, the taxane (e.g., paclitaxel) or the mTOR inhibitor (e.g., rapamycin) in the nanoparticles are coated with the carrier protein (e.g., albumin). In some embodiments, the weight ratio of the carrier protein (e.g., albumin) and the taxane (e.g., paclitaxel) or the mTOR inhibitor (e.g., rapamycin) in the nanoparticle composition is about 9:1 or less. In some embodiments, the albumin is human albumin. In some embodiments, the dose of the taxane (e.g., paclitaxel) or the mTOR inhibitor (e.g., rapamycin) in the nanoparticle composition is about 1 mg/m2 to about 150 mg/m2. In some embodiments, the dose of the taxane (e.g., paclitaxel) or the mTOR inhibitor (e.g., rapamvcin) in the nanoparticle composition is about 10 mg/m2 to about 50 mg/m2.

In some embodiments, there is provided a method of treating a cancer (e.g., a pancreatic cancer) in an individual comprising delivering to the individual a) an mRNA encoding a tumor suppressor protein, b) an RNAi (e.g., an siRNA) targeting an oncogene (e.g., KRAS), c) a nanoparticle composition comprising a taxane (e.g., paclitaxel) or an mTOR inhibitor (e.g., rapamycin) and a carrier protein (e.g., albumin), and d) an effective amount of gemcitabine. In some embodiments, the tumor suppressor protein is PTEN or p53. In some embodiments, the RNAi (e.g., siRNA) targets a mutant form of KRAS, wherein the mutant form of KRAS comprises a mutation on codon 12 or 61 of KRAS. In some embodiments, the mutant form of KRAS comprises G12D KRAS and/or G12C KRAS. In some embodiments, the individual has an aberration in a gene encoding the tumor suppressor protein. In some embodiments, the individual has an aberration in the oncogene. In some embodiments, the individual has an aberration in a gene encoding the tumor suppressor protein and the oncogene. In some embodiments, the mRNA encoding the tumor suppressor protein and the siRNA targeting the oncogene are delivered simultaneously. In some embodiments, the mRNA and the siRNA are delivered concurrently. In some embodiments, the mRNA and/or the siRNA are delivered intravenously. In some embodiments, the mRNA and/or the siRNA are complexed with a cell-penetrating peptide when delivered into the individual. In some embodiments, the cell-penetrating peptide is selected from the group consisting of CADY, PEP-1, PEP-2, MPG, VEPEP-3 peptides (used herein interchangeably with ADGN-103 peptides), VEPEP-4 peptides (used herein interchangeably with ADGN-104 peptides), VEPEP-5 peptides (used herein interchangeably with ADGN-105 peptides), VEPEP-6 peptides (used herein interchangeably with ADGN-106 peptides), VEPEP-9 peptides (used herein interchangeably with ADGN-109 peptides), and ADGN-100 peptides. In some embodiments, the cell-penetrating peptide is selected from ADGN 106 peptides and ADGN-100 peptides. In some embodiments, the mRNA (e.g., the mRNA encoding PTEN) and/or the siRNA (e.g., the siRNA targeting KRAS) are delivered about once a week or once every five days. In some embodiments, the dose of the mRNA (e.g., the mRNA encoding PTEN) for each administration in an individual is about 0.001 mg/kg to about 10 mg/kg (e.g., about 0.01 mg/kg to about 1 mg/kg, about 0.02 mg/kg to about 0.1 mg/kg). In some embodiments, the dose of the siRNA (e.g., the siRNA targeting KRAS) for each administration in an individual is about 0.001 mg/kg to about 10 mg/kg (e.g., about 0.01 mg/kg to about 1 mg/kg, about 0.02 mg/kg to about 0.1 mg/kg). In some embodiments, the dose of the mRNA (e.g., the mRNA encoding PTEN) for each administration in an individual is about 0.03 mg/m² to about 40 mg/m² (e.g., about 0.4 mg/m² to about 40 mg/m², about 0.8 mg/m² to about 4 mg/m²). In some embodiments, the dose of the siRNA (e.g., the siRNA targeting KRAS) for each administration in an individual is about 0.03 mg/m² to about 400 mg/m² (e.g., about 0.4 mg/m² to about 40 mg/m², about 0.8 mg/m² to about 4 mg/m²). In some embodiments, the individual is a human. In some embodiments, the nanoparticles comprising a taxane (e.g., paclitaxel) or an mTOR inhibitor (e.g., rapamycin) have an average diameter of no greater than 200 nm. In some embodiments, the taxane (e.g., paclitaxel) or the mTOR inhibitor (e.g., rapamycin) in the nanoparticles are coated with the carrier protein (e.g., albumin). In some embodiments, the weight ratio of the carrier protein (e.g., albumin) and the taxane (e.g., paclitaxel) or the mTOR inhibitor (e.g., rapamycin) in the nanoparticle composition is about 9:1 or less. In some embodiments, the albumin is human albumin. In some embodiments, the dose of the taxane (e.g., paclitaxel) or the mTOR inhibitor (e.g., rapamycin) in the nanoparticle composition is about 1 mg/m2 to about 150 mg/m2. In some embodiments, the dose of the taxane (e.g., paclitaxel) or the mTOR inhibitor (e.g., rapamycin) in the nanoparticle composition is about 10 mg/m2 to about 50 mg/m2.

D. Combination Therapy of an mRNA Complex/Nanoparticle with Another Agent

There is also provided a method of treating a disease or condition comprising delivering a) a complex or nanoparticle comprising an mRNA and a cell-penetrating peptide (CPP) as described herein, and b) another agent into a subject. In some embodiments, the mRNA encodes a therapeutic protein, for example, a tumor suppressor protein. In some embodiments, the mRNA encodes PTEN or p53. In some embodiments, the CPP is a VEPEP-3 peptide, a VEPEP-6 peptide, a VEPEP-9 peptide, or an ADGN-100 peptide. In some embodiments, the complex or nanoparticle further comprise a RNAi (such as siRNA). In some embodiments, the RNAi targets an endogenous gene, e.g., a disease-associated endogenous gene. In some embodiments, the RNAi targets an exogenous gene. In some embodiments, the disease associated endogenous gene is KRAS. In some embodiments, the other agent is nanoparticle composition comprising a taxane (e.g., paclitaxel) or an mTOR inhibitor (e.g., rapamycin) and a carrier protein (e.g., albumin) as described herein. In some embodiments, the other agent further comprises a chemotherapeutic agent (e.g., gemcitabine). In some embodiments, the other agent is nab-paclitaxel. In some embodiments, the disease or condition is a cancer. In some embodiments, the cancer is selected from pancreatic cancer, ovarian cancer, prostate cancer, and glioblastoma. In some embodiments, the cancer is pancreatic cancer. In some embodiments, the mRNA is modified (e.g., wherein at least one modified nucleoside is 5-methoxyuridine (5moU)).

Also provided herein is a method of treating a disease or condition comprising delivering a) a complex or nanoparticle comprising a mRNA (e.g., mRNA encoding a tumor suppressor protein, e.g., mRNA encoding PTEN or p53) and a cell-penetrating peptide (CPP) as described herein, b) a complex or nanoparticle comprising a RNAi and a CPP as described herein into a subject. In some embodiments, the RNAi is an siRNA or a miRNA (e.g., siRNA targeting an oncogene, e.g., siRNA targeting KRAS). In some embodiments, the RNAi is a therapeutic RNAi targeting a disease-associated endogenous gene or exogenous gene. In some embodiments, the mRNA complex or nanoparticle is delivered simultaneously with the RNAi complex or nanoparticle. In some embodiments, the mRNA complex or nanoparticle is delivered concurrently with the RNAi complex or nanoparticle. In some embodiments, the mRNA complex or nanoparticle is delivered sequentially with the RNAi complex or nanoparticle. In some embodiments, the mRNA complex or nanoparticle is delivered multiple times into the subject. In some embodiments, the RNAi complex or nanoparticle is delivered multiple times into the subject.

In some embodiments, there is provided a method of treating a disease or condition comprising delivering a complex or nanoparticle comprising a) a mRNA; b) a RNAi, and c) a cell-penetrating peptide (CPP) as described herein into a subject. In some embodiments, the RNAi is siRNA In some embodiments, the RNAi is miRNA. In some embodiments, the RNAi targets a disease associated endogenous or exogenous gene.

### E. Combination Therapy of Two or More RNAi

There is also provided a method of treating a disease or condition comprising delivering two or more RNA is (e.g., two or more siRNAs) into a subject. In some embodiments, the two or more siRNAs were complexed with a cell-penetrating peptide (CPP) as described herein. In some embodiments, the CPP is a VEPEP-3 peptide, a VEPEP-6 peptide, a VEPEP-9 peptide, or an ADGN-100 peptide. In some embodiments, the siRNA targets one or more oncogene. In some embodiments, the oncogene is KRAS. In some embodiments, the siRNA specifically targets a mutant form of KRAS, wherein the mutant form of KRAS has an aberration of KRAS selected from G12C, G12D and Q61K. In some embodiments, the siRNA comprises a cocktail of siRNAs comprising two or more siRNAs. In some embodiments, the two or more siRNAs comprises a first siRNA targeting a first mutant form of KRAS, and a second siRNA targeting a second mutant form of KRAS. Exemplary two or more siRNAs are not limited but include a) siRNAs targeting both KRAS G12C and KRAS G12D; b) siRNAs targeting both KRAS G12C and KRAS Q61K; c) siRNAs targeting both KRAS G12D and KRAS Q61K; and d) siRNAs targeting KRAS G12C, KRAS G12D and KRAS Q61K. In one preferred embodiment, the first siRNA targets KRAS G12C, and the second siRNA targets KRAS G12D. In some embodiments, the disease or condition is a cancer. In some embodiments, the cancer is selected from pancreatic cancer, ovarian cancer, prostate cancer, and glioblastoma. In some embodiments, the cancer is pancreatic cancer.

In some embodiments, there is provided a method of treating a cancer (e.g., a pancreatic cancer) comprising delivering into a subject an RNAi delivery complex comprising a) a first RNAi (e.g., a first siRNA) targeting a first gene, b) a second RNAi (e.g., a second siRNA) targeting a second gene, and c) a cell-penetrating peptide; wherein the first RNAi and/or the second RNAi is complexed with the cell-penetrating peptide. In some embodiments, the subject comprises an aberration in the first gene and/or the second gene. In some embodiments, the first gene and/or the second gene is an oncogene (e.g., KRAS). In some embodiments, the CPP is a VEPEP-3 peptide, a VEPEP-6 peptide, a VEPEP-9 peptide, or an ADGN-100 peptide.

In some embodiments, there is provided a method of treating a cancer (e.g., a pancreatic cancer) comprising delivering into a subject an RNAi delivery complex comprising a) a first RNAi (e.g., a first siRNA) targeting KRAS G12C, b) a second RNAi (e.g., a second siRNA) targeting KRAS G12D, and c) a cell-penetrating peptide; wherein the first RNAi and/or the second RNAi is complexed with the cell-penetrating peptide. In some embodiments, the subject comprises one or more aberration in KRAS. In some embodiments, the CPP is a VEPEP-3 peptide, a VEPEP-6 peptide, a VEPEP-9 peptide, or an ADGN-100 peptide. In some embodiments, the RNAi delivery complex further comprises a third RNAi (e.g., a third siRNA) targeting KRAS Q61K.

### Dosing and Method of Administering the Combination Therapy

In some embodiments, the mRNA/RNAi, the nanoparticle composition and/or the chemotherapeutic agent (e.g., gemcitabine) are administered simultaneously. In some embodiments, the mRNA/RNAi, the nanoparticle composition and/or the chemotherapeutic agent (e.g., gemcitabine) are administered sequentially. In some embodiments, the mRNA/RNAi, the nanoparticle composition and/or the chemotherapeutic agent (e.g., gemcitabine) are administered concurrently.

The dosing frequency of the mRNA, RNAi, nanoparticle composition and/or the chemotherapeutic agent may be adjusted over the course of the treatment, based on the judgment of the administering physician. When administered separately, the mRNA, RNAi, nanoparticle composition and/or the chemotherapeutic agent can be administered at different dosing frequency or intervals. In some embodiments, sustained continuous release formulation of the mRNA, RNAi, nanoparticle composition and/or the chemotherapeutic agent may be used. Various formulations and devices for achieving sustained release are known in the art. A combination of the administration configurations described herein can also be used.

The mRNA, RNAi, nanoparticle composition and/or the chemotherapeutic agent can be administered using the same route of administration or different routes of administration.

In some embodiments, the mRNA or RNAi (e.g., siRNA) or the mRNA delivery complex or RNAi delivery complex as described herein is formulated for systemic or tropical administration. In some embodiments, the mRNA or RNAi (e.g., siRNA) or the mRNA delivery complex or RNAi delivery complex as described herein is formulated for intravenous, intratumoral, intraarterial, topical, intraocular, ophthalmic, intraportal, intracranial, intracerebral, intracerebroventricular, intrathecal, intravesicular, intradermal, subcutaneous, intramuscular, intranasal, intratracheal, pulmonary, intracavity, or oral administration.

In some embodiments, dosages of the mRNA and/or the RNAi (e.g., siRNA) for treatment of human or mammalian subjects are in the range of about 0.001 mg/kg to about 100 mg/kg for each administration. In some embodiments, the exemplary dosage of the mRNA (e.g., PTEN mRNA and/or p53 mRNA) is about 0.005 mg/kg to about 0.5 mg/kg (e.g., about 0.01 mg/kg to about 0.05 mg/kg, about 0.02 mg/kg to about 0.04 mg/kg) for each administration in the individual. In some embodiments, the exemplary dosage of the RNAi (e.g., KRAS siRNA) is about 0.005 mg/kg to about 0.5 mg/kg (e.g., about 0.01 mg/kg to about 0.1 mg/kg, e.g., about 0.3 mg/kg to about 0.5 mg/kg, e.g., about 0.04 mg/kg) for each administration in the individual. In some embodiments, the individual is a human being.

In some embodiments, dosages of the mRNA and/or the RNAi (e.g., siRNA) for treatment of human or mammalian subjects are in the range of about 0.03 mg/m² to about 4000 mg/m² for each administration. In some embodiments, the exemplary dosage of the mRNA (e.g., PTEN mRNA and/or p53 mRNA) is about 0.01 mg/m² to about 20 mg/m² (e.g., about 0.2 mg/m² to about 2 mg/m², about 0.5 mg/m² to about 1.5 mg/m²) for each administration in the individual. In some embodiments, the exemplary dosage of the RNAi (e.g., KRAS siRNA) is about 0.2 mg/m² to about 20 mg/m² (e.g., about 0.4 mg/m² to about 4 mg/m², e.g., about 1 mg/m² to about 20 mg/m², e.g., about 1.5 mg/m²) for each administration in the individual. In some embodiments, the individual is a human being.

Exemplary dosing frequencies of the mRNA and/or RNAi include, but are not limited to, weekly without break; weekly, three out of four weeks: once every three weeks; once every two weeks; weekly, two out of three weeks. In some embodiments, the mRNA and/or RNAi (e.g., siRNA) is administered about once every 2 weeks, once every 3 weeks, once every 4 weeks, once every 6 weeks, or once every 8 weeks. In some embodiments, the mRNA and/or RNAi (e.g., siRNA) is administered at least about any of 1×, 2×, 3×, 4×, 5×, 6×, or 7× (i.e., daily) a week. In some embodiments, the intervals between each administration are less than about any of 6 months, 3 months, 1 month, 20 days, 15, days, 12 days, 10 days, 9 days, 8 days, 7 days, 6 days, 5 days, 4 days, 3 days, 2 days, or 1 day. In some embodiments, the intervals between each administration are more than about any of 1 month, 2 months, 3 months, 4 months, 5 months, 6 months, 8 months, or 12 months. In some embodiments, there is no break in the dosing schedule. In some embodiments, the interval between each administration is no more than about a week. In some embodiments, the schedule of administration of the mRNA and/or RNAi (e.g., siRNA) to an individual ranges from a single administration that constitutes the entire treatment to daily administration. The administration of the mRNA and/or RNAi (e.g., siRNA) can be extended over an extended period of time, such as from about a month up to about seven years. In some embodiments, the mRNA and/or RNAi (e.g., siRNA) is administered over a period of at least about any of 2, 3, 4, 5, 6, 7, 8, 9, 10, 11, 12, 18, 24, 30, 36, 48, 60, 72, or 84 months.

The doses required for the mRNA. RNAi, the taxane and/or the chemotherapeutic agent may (but not necessarily) be lower than what is normally required when each agent is administered alone. Thus, in some embodiments, a subtherapeutic amount of the mRNA. RNAi, nanoparticle composition and/or the chemotherapeutic agent is administered. “Subtherapeutic amount” or “subtherapeutic level” refer to an amount that is less than the therapeutic amount, that is, less than the amount normally used when the drug in the nanoparticle composition and/or the other agent are administered alone. The reduction may be reflected in terms of the amount administered at a given administration and/or the amount administered over a given period of time (reduced frequency).

In some embodiments, the dose of both the mRNA, RNAi, taxane in the nanoparticle composition and/or the chemotherapeutic agent are reduced as compared to the corresponding normal dose of each when administered alone. In some embodiments, the mRNA, RNAi, nanoparticle composition and/or the chemotherapeutic agent are administered at a subtherapeutic, i.e., reduced, level. In some embodiments, the dose of the mRNA, RNAi, nanoparticle composition and/or the chemotherapeutic agent is substantially less than the established maximum toxic dose (MTD). For example, the dose of the nanoparticle composition and/or the other agent is less than about 50%, 40%, 30%, 20%, or 10% of the MTD.

A combination of the administration configurations described herein can be used. The combination therapy methods described herein may be performed alone or in conjunction with another therapy, such as chemotherapy, radiation therapy, surgery, hormone therapy, gene therapy, immunotherapy, chemoimmunotherapy, hepatic artery-based therapy, cryotherapy, ultrasound therapy, liver transplantation, local ablative therapy, radiofrequency ablation therapy, photodynamic therapy, and the like. Additionally, a person having a greater risk of developing a cancer (e.g., a pancreatic cancer) may receive treatments to inhibit or and/or delay the development of the disease.

### Nanoparticle Compositions

The dose of the nanoparticle compositions administered to an individual (such as a human) may vary with the particular composition, the mode of administration, and the type of pancreatic cancer being treated. In some embodiments, the amount of the composition is effective to result in an objective response (such as a partial response, a complete response, or stable disease). In some embodiments, the amount of the nanoparticle composition is sufficient to result in a complete response in the individual. In some embodiments, the amount of the nanoparticle composition is sufficient to result in a partial response in the individual. In some embodiments, the amount of the nanoparticle composition is sufficient to result in stable disease (i.e., pancreatic cancer) in the individual. In some embodiments, the amount of the nanoparticle composition administered (for example when administered alone) is sufficient to produce an overall response rate of more than about any of 25%, 30%, 32%, 35%, 36%, 37%, 38%, 39%, 40%, 50%, 60%, 65%, or 70% among a population of individuals treated with the nanoparticle composition. Responses of an individual to the treatment of the methods described herein can be determined, for example, based on RECIST levels.

In some embodiments, the amount of the composition is sufficient to prolong progress-free survival of the individual. In some embodiments, the amount of the composition is sufficient to prolong overall survival of the individual. In some embodiments, the amount of the composition (for example when administered along) is sufficient to produce clinical benefits of more than about any of 25%, 30%, 32%, 35%, 36%, 37%, 38%, 39%, 40%, 50%, 60%, 65%, or 70% among a population of individuals treated with the nanoparticle composition.

In some embodiments, the amount of the composition, first therapy, second therapy, or combination therapy is an amount sufficient to decrease the size of a tumor, decrease the number of cancer cells, or decrease the growth rate of a tumor by at least about any of 10%, 20%, 30%, 40%, 50%, 60%, 70%, 80%, 90%, 95% or 100% compared to the corresponding tumor size, number of pancreatic cancer cells, or tumor growth rate in the same subject prior to treatment or compared to the corresponding activity in other subjects not receiving the treatment. Standard methods can be used to measure the magnitude of this effect, such as in vitro assays with purified enzyme, cell-based assays, animal models, or human testing.

In some embodiments, the amount of the taxane (e.g., paclitaxel) or the mTOR inhibitor (e.g., rapamycin) in the composition is below the level that induces a toxicological effect (i.e., an effect above a clinically acceptable level of toxicity) or is at a level where a potential side effect can be controlled or tolerated when the composition is administered to the individual.

In some embodiments, the amount of the composition is close to a maximum tolerated dose (MTD) of the composition following the same dosing regime. In some embodiments, the amount of the composition is more than about any of 80%, 90%, 95%, or 98% of the MTD.

Exemplary effective amounts of a taxane (e.g., paclitaxel) or an mTOR inhibitor (e.g., rapamycin) in the nanoparticle composition include, but not limited to, about 1 mg/m² to 150 mg/m² of a taxane (e.g., paclitaxel) or an mTOR inhibitor (e.g., rapamycin) for each administration.

Exemplary dosing frequencies for the administration of the nanoparticle compositions include, but are not limited to, daily, every two days, every three days, every four days, every five days, every six days, weekly without break, three out of four weeks, once every three weeks, once every two weeks, or two out of three weeks. In some embodiments, the composition is administered about once every 2 weeks, once every 3 weeks, once every 4 weeks, once every 6 weeks, or once every 8 weeks. In some embodiments, the composition is administered at least about any of 1×, 2×, 3×, 4×, 5×, 6×, or 7× (i.e., daily) a week. In some embodiments, the intervals between each administration are less than about any of 6 months, 3 months, 1 month, 28 days, 20 days, 15, days, 14 days, 13 days, 12 days, 11 days, 10 days, 9 days, 8 days, 7 days, 6 days, 5 days, 4 days, 3 days, 2 days, or 1 day. In some embodiments, the intervals between each administration are more than about any of 1 month, 2 months, 3 months, 4 months, 5 months, 6 months, 8 months, or 12 months. In some embodiments, there is no break in the dosing schedule. In some embodiments, the interval between each administration is no more than about a week.

In some embodiments, the dosing frequency is once every two days for one time, two times, three times, four times, five times, six times, seven times, eight times, nine times, ten times, and eleven times. In some embodiments, the dosing frequency is once every two days for five times. In some embodiments, the taxane (e.g., paclitaxel) or the mTOR inhibitor (e.g., rapamycin) is administered over a period of at least ten days, wherein the interval between each administration is no more than about two days, and wherein the dose of the taxane (e.g., paclitaxel) or the mTOR inhibitor (e.g., rapamycin) at each administration is about 1 mg/m² to about 150 mg/m².

In some embodiments, the taxane (e.g., paclitaxel) or the mTOR inhibitor (e.g., rapamycin) is administered on days 1, 8, and 15 on a 28-day cycle, wherein the dose of the taxane (e.g., paclitaxel) or the mTOR inhibitor (e.g., rapamycin) at each administration is about 1 mg/m² to about 150 mg/m². In some embodiments, the taxane (e.g., paclitaxel) or the mTOR inhibitor (e.g., rapamycin) is administered intravenously over 30 minutes on days 1, 8, and 15 on a 28-day cycle, wherein the dose of the taxane (e.g., paclitaxel) or the mTOR inhibitor (e.g., rapamycin) at each administration is about 1 mg/m² to about 150 mg/m². In some embodiments, the taxane is paclitaxel.

The administration of the composition can be extended over an extended period of time, such as from about a month up to about seven years. In some embodiments, the composition is administered over a period of at least about any of 2, 3, 4, 5, 6, 7, 8, 9, 10, 11, 12, 18, 24, 30, 36, 48, 60, 72, or 84 months.

In some embodiments, the dosage of a taxane (e.g., paclitaxel) or an mTOR inhibitor (e.g., rapamycin) in a nanoparticle composition can be in the range of 5-150 mg/m² (such as 80-150 mg/m², for example 100-120 mg/m²) when given on a weekly schedule.

Other exemplary dosing schedules for the administration of the nanoparticle composition (e.g., paclitaxel/albumin nanoparticle composition) include, but are not limited to, 100 mg/m², weekly, without break; 75 mg/m² weekly, 3 out of 4 weeks: 100 mg/m², weekly, 3 out of 4 weeks: 125 mg/m², weekly, 3 out of 4 weeks; 125 mg/m², weekly, 2 out of 3 weeks; 130 mg/m², weekly, without break; and 20-150 mg/m² twice a week. The dosing frequency of the composition may be adjusted over the course of the treatment based on the judgment of the administering physician.

In some embodiments, the individual is treated for at least about any of one, two, three, four, five, six, seven, eight, nine, or ten treatment cycles.

The compositions described herein allow infusion of the composition to an individual over an infusion time that is shorter than about 24 hours. For example, in some embodiments, the composition is administered over an infusion period of less than about any of 24 hours, 12 hours, 8 hours, 5 hours, 3 hours, 2 hours, 1 hour, 30 minutes, 20 minutes, or 10 minutes. In some embodiments, the composition is administered over an infusion period of about 30 minutes.

Other exemplary dose of the taxane (in some embodiments paclitaxel) in the nanoparticle composition include, but is not limited to, about any of 50 mg/m², 60 mg/m², 75 mg/m², 80 mg/m², 90 mg/m², 100 mg/m², 120 mg/m², and 150 mg/m². For example, the dosage of paclitaxel in a nanoparticle composition can be in the range of about 50-150 mg/m² when given on a weekly schedule.

The nanoparticle compositions can be administered to an individual (such as human) via various routes, including, for example, intravenous, intra-arterial, intraperitoneal, intrapulmonary, oral, inhalation, intravesicular, intramuscular, intra-tracheal, subcutaneous, intraocular, intrathecal, transmucosal, and transdermal. In some embodiments, sustained continuous release formulation of the composition may be used. In some embodiments, the composition is administered intravenously. In some embodiments, the composition is administered intraarterially. In some embodiments, the composition is administered intraperitoneally.

### The Chemotherapeutic Agent (e.g., Gemcitabine)

The chemotherapeutic agent (e.g., gemcitabine) described herein can be administered to an individual (such as human) via various routes, such as parenterally, including intravenous, intra-arterial, intraperitoneal, intrapulmonary, oral, inhalation, intravesicular, intramuscular, intra-tracheal, subcutaneous, intraocular, intrathecal, or transdermal. In some embodiments, the chemotherapeutic agent (e.g., gemcitabine) is administrated intravenously.

The dosing frequency of the chemotherapeutic agent (e.g., gemcitabine) can be the same or different from that of the mRNA, RNAi or nanoparticle composition. Exemplary frequencies are provided above. As further example, the the chemotherapeutic agent (e.g., gemcitabine) can be administered three times a day, two times a day, daily, 6 times a week, 5 times a week, 4 times a week, 3 times a week, two times a week, weekly. In some embodiments, the chemotherapeutic agent (e.g., gemcitabine) is administered twice daily or three times daily. Exemplary amounts of the chemotherapeutic agent (e.g., gemcitabine) include, but are not limited to, any of the following ranges: about 0.5 to about 5 mg, about 5 to about 10 mg, about 10 to about 15 mg, about 15 to about 20 mg, about 20 to about 25 mg, about 20 to about 50 mg, about 25 to about 50 mg, about 50 to about 75 mg, about 50 to about 100 mg, about 75 to about 100 mg, about 100 to about 125 mg, about 125 to about 150 mg, about 150 to about 175 mg, about 175 to about 200 mg, about 200 to about 225 mg, about 225 to about 250 mg, about 250 to about 300 mg, about 300 to about 350 mg, about 350 to about 400 mg, about 400 to about 450 mg, or about 450 to about 500 mg. For example, the chemotherapeutic agent (e.g., gemcitabine) can be administered at a dose of about 1 mg/kg to about 200 mg/kg (including for example about 1 mg/kg to about 20 mg/kg, about 20 mg/kg to about 40 mg/kg, about 40 mg/kg to about 60 mg/kg, about 60 mg/kg to about 80 mg/kg, about 80 mg/kg to about 100 mg/kg, about 100 mg/kg to about 120 mg/kg, about 120 mg/kg to about 140 mg/kg, about 140 mg/kg to about 200 mg/kg).

In some embodiments, the effective amount of taxane in the nanoparticle composition is between about 45 mg/m² to about 350 mg/m² and the effective amount of the other chemotherapeutic agent (e.g., gemcitabine) is about 1 mg/kg to about 200 mg/kg (including for example about 1 mg/kg to about 20 mg/kg, about 20 mg/kg to about 40 mg/kg, about 40 mg/kg to about 60 mg/kg, about 60 mg/kg to about 80 mg/kg, about 80 mg/kg to about 100 mg/kg, about 100 mg/kg to about 120 mg/kg, about 120 mg/kg to about 140 mg/kg, about 140 mg/kg to about 200 mg/kg). In some embodiments, the effective amount of taxane in the nanoparticle composition is between about 80 mg/m² to about 350 mg/m² and the effective amount of the other chemotherapeutic agent (e.g., gemcitabine) is about 1 mg/kg to about 200 mg/kg (including for example about 1 mg/kg to about 20 mg/kg, about 20 mg/kg to about 40 mg/kg, about 40 mg/kg to about 60 mg/kg, about 60 mg/kg to about 80 mg/kg, about 80 mg/kg to about 100 mg/kg, about 100 mg/kg to about 120 mg/kg, about 120 mg/kg to about 140 mg/kg, about 140 mg/kg to about 200 mg/kg). In some embodiments, the effective amount of taxane in the nanoparticle composition is between about 80 mg/m² to about 300 mg/m² and the effective amount of the other chemotherapeutic agent (e.g., gemcitabine) is about 1 mg/kg to about 200 mg/kg (including for example about 1 mg/kg to about 20 mg/kg, about 20 mg/kg to about 40 mg/kg, about 40 mg/kg to about 60 mg/kg, about 60 mg/kg to about 80 mg/kg, about 80 mg/kg to about 100 mg/kg, about 100 mg/kg to about 120 mg/kg, about 120 mg/kg to about 140 mg/kg, about 140 mg/kg to about 200 mg/kg). In some embodiments, the effective amount of taxane in the nanoparticle composition is between about 150 mg/m² to about 350 mg/m² and the effective amount of the other chemotherapeutic agent (e.g., gemcitabine) is about 1 mg/kg to about 200 mg/kg (including for example about 1 mg/kg to about 20 mg/kg, about 20 mg/kg to about 40 mg/kg, about 40 mg/kg to about 60 mg/kg, about 60 mg/kg to about 80 mg/kg, about 80 mg/kg to about 100 mg/kg, about 100 mg/kg to about 120 mg/kg, about 120 mg/kg to about 140 mg/kg, about 140 mg/kg to about 200 mg/kg). In some embodiments, the effective amount of taxane in the nanoparticle composition is between about 80 mg/m² to about 150 mg/m² and the effective amount of the other chemotherapeutic agent (e.g., gemcitabine) is about 1 mg/kg to about 200 mg/kg (including for example about 1 mg/kg to about 20 mg/kg, about 20 mg/kg to about 40 mg/kg, about 40 mg/kg to about 60 mg/kg, about 60 mg/kg to about 80 mg/kg, about 80 mg/kg to about 100 mg/kg, about 100 mg/kg to about 120 mg/kg, about 120 mg/kg to about 140 mg/kg, about 140 mg/kg to about 200 mg/kg). In some embodiments, the effective amount of taxane (e.g., paclitaxel) in the nanoparticle composition is about 100 mg/m². In some embodiments, the effective amount of taxane in the nanoparticle composition is between about 170 mg/m² to about 200 mg/m² and the effective amount of the other chemotherapeutic agent (e.g., gemcitabine) is about 1 mg/kg to about 200 mg/kg (including for example about 1 mg/kg to about 20 mg/kg, about 20 mg/kg to about 40 mg/kg, about 40 mg/kg to about 60 mg/kg, about 60 mg/kg to about 80 mg/kg, about 80 mg/kg to about 100 mg/kg, about 100 mg/kg to about 120 mg/kg, about 120 mg/kg to about 140 mg/kg, about 140 mg/kg to about 200 mg/kg). In some embodiments, the effective amount of taxane in the nanoparticle composition is between about 200 mg/m² to about 350 mg/m² and the effective amount of the other chemotherapeutic agent (e.g., gemcitabine) is about 1 mg/kg to about 200 mg/kg (including for example about 1 mg/kg to about 20 mg/kg, about 20 mg/kg to about 40 mg/kg, about 40 mg/kg to about 60 mg/kg, about 60 mg/kg to about 80 mg/kg, about 80 mg/kg to about 100 mg/kg, about 100 mg/kg to about 120 mg/kg, about 120 mg/kg to about 140 mg/kg, about 140 mg/kg to about 200 mg/kg). In some embodiments, the effective amount of taxane (e.g., paclitaxel) in the nanoparticle composition is about 260 mg/m². In some embodiments of any of the above methods, the effective amount of the other chemotherapeutic agent (e.g., gemcitabine) is about 20-30 mg/kg, about 30-40 mg/kg, about 40-50 mg/kg, about 50-60 mg/kg, about 60-70 mg/kg, about 70-80 mg/kg, about 80-100 mg/kg, or about 100-120 mg/kg.

In some embodiments, the effective amount of taxane (e.g., paclitaxel) in the nanoparticle composition is between about 30 to about 300 mg/m² and the effective amount of the other chemotherapeutic agent (e.g., gemcitabine) is between about 100 to about 5000 mg/m². In some embodiments, the effective amount of taxane (e.g., paclitaxel) in the nanoparticle composition is about any of 30, 50, 75, 100, 125, 150, 175, 200, 225, 250, 275, or 300 mg/m² and the effective amount of the other chemotherapeutic agent (e.g., gemcitabine) is about any of 100, 200, 300, 400, 500, 600, 700, 800, 900, 1000, 1250, 1500, 1750, 2000, 2250, 2500, 2750, 3000, 3250, 3500, 3750, 4000, 4250, 4500, 4750, or 5000 mg/m². In some embodiments, the nanoparticle composition is about 30 to about 300 mg/m² and the effective amount of the other chemotherapeutic agent (e.g., gemcitabine) is about 100 to about 5000 mg/m², wherein the nanoparticle composition and the other chemotherapeutic agent (e.g., gemcitabine) are both administered weekly to the individual who has been previously treated for pancreatic cancer. In some embodiments, the nanoparticle composition is about 30 to about 300 mg/m² and the effective amount of the other chemotherapeutic agent (e.g., gemcitabine) is about 100 to about 5000 mg/m², wherein the nanoparticle composition and the other chemotherapeutic agent (e.g., gemcitabine) are both administered at a frequency of less than weekly to the individual who has been previously treated for pancreatic cancer. In some embodiments, the nanoparticle composition is about 30 to about 300 mg/m² and the effective amount of the other chemotherapeutic agent (e.g., gemcitabine) is about 100 to about 5000 mg/m², wherein the nanoparticle composition and the other chemotherapeutic agent (e.g., gemcitabine) are both administered intravenously over 30 minutes on days 1, 8, and 15 on a 28-day cycle to the individual.

### Methods of Treatment Based on Presence of a Biomarker

The present invention in one aspect provides methods of treating a disease or condition in an individual comprising delivering to the individual an mRNA and/or a RNAi (e.g., siRNA) based on the status of one or more aberrations. In some embodiments, the aberration is in the gene corresponding to the mRNA and/or the gene targeted by the RNAi (e.g, siRNA). In some embodiments, the aberration is in the protein corresponding to the mRNA and/or the protein corresponding to the gene targeted by the RNAi (e.g., siRNA).

In some embodiments, there is provided a method of treating a cancer in an individual, comprising delivering to the individual a) an mRNA encoding a tumor suppressor protein and b) an siRNA targeting an oncogene, wherein the individual comprises an aberration in a gene encoding the tumor suppressor protein and/or the oncogene. In some embodiments, the cancer is selected from pancreatic cancer, ovarian cancer, prostate cancer, and glioblastoma. In some embodiments, the cancer is pancreatic cancer. In some embodiments, the tumor suppressor protein is PTEN. In some embodiments, the oncogene is KRAS. In some embodiments, the individual has an aberration in a gene encoding the tumor suppressor protein and the oncogene. In some embodiments, the mRNA encoding the tumor suppressor protein and the siRNA targeting the oncogene are delivered simultaneously. In some embodiments, the mRNA and the siRNA are delivered concurrently. In some embodiments, the mRNA and/or the siRNA are delivered intravenously. In some embodiments, the mRNA and/or the siRNA are complexed with a cell-penetrating peptide when delivered into the individual. In some embodiments, the cell-penetrating peptide is selected from the group consisting of CADY, PEP-1, PEP-2, MPG, VEPEP-3 peptides (used herein interchangeably with ADGN-103 peptides), VEPEP-4 peptides (used herein interchangeably with ADGN-104 peptides), VEPEP-5 peptides (used herein interchangeably with ADGN-105 peptides), VEPEP-6 peptides (used herein interchangeably with ADGN-106 peptides), VEPEP-9 peptides (used herein interchangeably with ADGN-109 peptides), and ADGN-100 peptides. In some embodiments, the cell-penetrating peptide is selected from ADGN 106 peptides and ADGN-100 peptides. In some embodiments, the mRNA (e.g., the mRNA encoding PTEN) and/or the siRNA (e.g., the siRNA targeting KRAS) are delivered about once a week or once every five days. In some embodiments, the dose of the mRNA (e.g., the mRNA encoding PTEN) for each administration in an individual is about 0.001 mg/kg to about 10 mg/kg (e.g., about 0.01 mg/kg to about 1 mg/kg, about 0.02 mg/kg to about 0.1 mg/kg). In some embodiments, the dose of the siRNA (e.g., the siRNA targeting KRAS) for each administration in an individual is about 0.001 mg/kg to about 10 mg/kg (e.g., about 0.01 mg/kg to about 1 mg/kg, about 0.02 mg/kg to about 0.1 mg/kg). In some embodiments, the dose of the mRNA (e.g., the mRNA encoding PTEN) for each administration in an individual is about 0.03 mg/m² to about 400 mg/m² (e.g., about 0.4 mg/m² to about 40 mg/m², about 0.8 mg/m² to about 4 mg/m²). In some embodiments, the dose of the siRNA (e.g., the siRNA targeting KRAS) for each administration in an individual is about 0.03 mg/m² to about 400 mg/m² (e.g., about 0.4 mg/m² to about 40 mg/m², about 0.8 mg/m² to about 4 mg/m²). In some embodiments, the individual is a human.

In some embodiments, there is provided a method of treating a cancer in an individual, comprising a) assessing an aberration of a gene encoding a tumor suppressor protein and/or an oncogene, and b) delivering to the individual i) an mRNA encoding the tumor suppressor protein and b) an siRNA targeting the oncogene. In some embodiments, the cancer is selected from pancreatic cancer, ovarian cancer, prostate cancer, and glioblastoma. In some embodiments, the cancer is pancreatic cancer. In some embodiments, the tumor suppressor protein is PTEN. In some embodiments, the oncogene is KRAS. In some embodiments, the individual has an aberration in a gene encoding the tumor suppressor protein. In some embodiments, the individual has an aberration in the oncogene. In some embodiments, the individual has an aberration in a gene encoding the tumor suppressor protein and the oncogene. In some embodiments, the mRNA encoding the tumor suppressor protein and the siRNA targeting the oncogene are delivered simultaneously. In some embodiments, the mRNA and the siRNA are delivered concurrently. In some embodiments, the mRNA and/or the siRNA are delivered intravenously. In some embodiments, the mRNA and/or the siRNA are complexed with a cell-penetrating peptide when delivered into the individual. In some embodiments, the cell-penetrating peptide is selected from the group consisting of CADY, PEP-1, PEP-2, MPG, VEPEP-3 peptides (used herein interchangeably with ADGN-103 peptides), VEPEP-4 peptides (used herein interchangeably with ADGN-104 peptides), VEPEP-5 peptides (used herein interchangeably with ADGN-105 peptides), VEPEP-6 peptides (used herein interchangeably with ADGN-106 peptides), VEPEP-9 peptides (used herein interchangeably with ADGN-109 peptides), and ADGN-100 peptides. In some embodiments, the cell-penetrating peptide is selected from ADGN 106 peptides and ADGN-100 peptides. In some embodiments, the mRNA (e.g., the mRNA encoding PTEN) and/or the siRNA (e.g., the siRNA targeting KRAS) are delivered about once a week or once every five days. In some embodiments, the dose of the mRNA (e.g., the mRNA encoding PTEN) for each administration in an individual is about 0.001 mg/kg to about 10 mg/kg (e.g., about 0.01 mg/kg to about 1 mg/kg, about 0.02 mg/kg to about 0.1 mg/kg). In some embodiments, the dose of the siRNA (e.g., the siRNA targeting KRAS) for each administration in an individual is about 0.001 mg/kg to about 10 mg/kg (e.g., about 0.01 mg/kg to about 1 mg/kg, about 0.02 mg/kg to about 0.1 mg/kg). In some embodiments, the dose of the mRNA (e.g., the mRNA encoding PTEN) for each administration in an individual is about 0.03 mg/m² to about 400 mg/m² (e.g., about 0.4 mg/m² to about 40 mg/m², about 0.8 mg/m² to about 4 mg/m²). In some embodiments, the dose of the siRNA (e.g., the siRNA targeting KRAS) for each administration in an individual is about 0.03 mg/m² to about 400 mg/m² (e.g., about 0.4 mg/m² to about 40 mg/m², about 0.8 mg/m² to about 4 mg/m²). In some embodiments, the individual is a human.

In some embodiments, there is provided a method of treating a cancer in an individual, comprising a) assessing an aberration of a gene encoding a tumor suppressor protein and/or an oncogene, and b) delivering to the individual i) an mRNA encoding the tumor suppressor protein and ii) an siRNA targeting the oncogene, wherein the individual is selected for treatment based on having the aberration in the gene encoding the tumor suppressor protein and/or the oncogene. In some embodiments, the cancer is selected from pancreatic cancer, ovarian cancer, prostate cancer, and glioblastoma. In some embodiments, the cancer is pancreatic cancer. In some embodiments, the tumor suppressor protein is PTEN. In some embodiments, the oncogene is KRAS. In some embodiments, the individual has an aberration in a gene encoding the tumor suppressor protein. In some embodiments, the individual has an aberration in the oncogene. In some embodiments, the individual has an aberration in a gene encoding the tumor suppressor protein and the oncogene. In some embodiments, the mRNA encoding the tumor suppressor protein and the siRNA targeting the oncogene are delivered simultaneously. In some embodiments, the mRNA and the siRNA are delivered concurrently. In some embodiments, the mRNA and/or the siRNA are delivered intravenously. In some embodiments, the mRNA and/or the siRNA are complexed with a cell-penetrating peptide when delivered into the individual. In some embodiments, the cell-penetrating peptide is selected from the group consisting of CADY, PEP-1, PEP-2, MPG, VEPEP-3 peptides (used herein interchangeably with ADGN-103 peptides). VEPEP-4 peptides (used herein interchangeably with ADGN-104 peptides). VEPEP-5 peptides (used herein interchangeably with ADGN-105 peptides), VEPEP-6 peptides (used herein interchangeably with ADGN-106 peptides), VEPEP-9 peptides (used herein interchangeably with ADGN-109 peptides), and ADGN-100 peptides. In some embodiments, the cell-penetrating peptide is selected from ADGN 106 peptides and ADGN-100 peptides. In some embodiments, the mRNA (e.g., the mRNA encoding PTEN) and/or the siRNA (e.g., the siRNA targeting KRAS) are delivered about once a week or once every five days. In some embodiments, the dose of the mRNA (e.g., the mRNA encoding PTEN) for each administration in an individual is about 0.001 mg/kg to about 10 mg/kg (e.g., about 0.01 mg/kg to about 1 mg/kg, about 0.02 mg/kg to about 0.1 mg/kg). In some embodiments, the dose of the siRNA (e.g., the siRNA targeting KRAS) for each administration in an individual is about 0.001 mg/kg to about 10 mg/kg (e.g., about 0.01 mg/kg to about 1 mg/kg, about 0.02 mg/kg to about 0.1 mg/kg). In some embodiments, the dose of the mRNA (e.g., the mRNA encoding PTEN) for each administration in an individual is about 0.03 mg/m² to about 400 mg/m² (e.g., about 0.4 mg/m² to about 40 mg/m², about 0.8 mg/m² to about 4 mg/m²). In some embodiments, the dose of the siRNA (e.g., the siRNA targeting KRAS) for each administration in an individual is about 0.03 mg/m² to about 400 mg/m² (e.g., about 0.4 mg/m² to about 40 mg/m², about 0.8 mg/m² to about 4 mg/m²). In some embodiments, the individual is a human.

In some embodiments, there is provided a method of treating a cancer in an individual, comprising a) selecting (e.g., identifying or recommending) the individual for treatment based on the individual having an aberration in the gene encoding the tumor suppressor protein and/or the oncogene, and b) delivering to the individual i) an mRNA encoding the tumor suppressor protein and ii) an siRNA targeting the oncogene. In some embodiments, the cancer is selected from pancreatic cancer, ovarian cancer, prostate cancer, and glioblastoma. In some embodiments, the cancer is pancreatic cancer. In some embodiments, the tumor suppressor protein is PTEN. In some embodiments, the oncogene is KRAS. In some embodiments, the individual has an aberration in a gene encoding the tumor suppressor protein. In some embodiments, the individual has an aberration in the oncogene. In some embodiments, the individual has an aberration in a gene encoding the tumor suppressor protein and the oncogene. In some embodiments, the mRNA encoding the tumor suppressor protein and the siRNA targeting the oncogene are delivered simultaneously. In some embodiments, the mRNA and the siRNA are delivered concurrently. In some embodiments, the mRNA and/or the siRNA are delivered intravenously. In some embodiments, the mRNA and/or the siRNA are complexed with a cell-penetrating peptide when delivered into the individual. In some embodiments, the cell-penetrating peptide is selected from the group consisting of CADY, PEP-1, PEP-2, MPG, VEPEP-3 peptides (used herein interchangeably with ADGN-103 peptides), VEPEP-4 peptides (used herein interchangeably with ADGN-104 peptides), VEPEP-5 peptides (used herein interchangeably with ADGN-105 peptides), VEPEP-6 peptides (used herein interchangeably with ADGN-106 peptides), VEPEP-9 peptides (used herein interchangeably with ADGN-109 peptides), and ADGN-100 peptides. In some embodiments, the cell-penetrating peptide is selected from ADGN 106 peptides and ADGN-100 peptides. In some embodiments, the mRNA (e.g., the mRNA encoding PTEN) and/or the siRNA (e.g., the siRNA targeting KRAS) are delivered about once a week or once every five days. In some embodiments, the dose of the mRNA (e.g., the mRNA encoding PTEN) for each administration in an individual is about 0.001 mg/kg to about 10 mg/kg (e.g., about 0.01 mg/kg to about 1 mg/kg, about 0.02 mg/kg to about 0.1 mg/kg). In some embodiments, the dose of the siRNA (e.g., the siRNA targeting KRAS) for each administration in an individual is about 0.001 mg/kg to about 10 mg/kg (e.g., about 0.01 mg/kg to about 1 mg/kg, about 0.02 mg/kg to about 0.1 mg/kg). In some embodiments, the dose of the mRNA (e.g., the mRNA encoding PTEN) for each administration in an individual is about 0.03 mg/m² to about 400 mg/m² (e.g., about 0.4 mg/m² to about 40 mg/m² about 0.8 mg/m² to about 4 mg/m²). In some embodiments, the dose of the siRNA (e.g., the siRNA targeting KRAS) for each administration in an individual is about 0.03 mg/m² to about 400 mg/m² (e.g., about 0.4 mg/m² to about 40 mg/m², about 0.8 mg/m² to about 4 mg/m²). In some embodiments, the individual is a human.

In some embodiments, there is provided a method of treating a cancer in an individual, comprising a) assessing an aberration of a gene encoding a tumor suppressor protein and/or an oncogene, b) selecting (e.g., identifying or recommending) the individual for treatment based on the individual having an aberration in the gene encoding the tumor suppressor protein and/or the oncogene, and c) delivering to the individual i) an mRNA encoding the tumor suppressor protein and ii) an siRNA targeting the oncogene. In some embodiments, the cancer is selected from pancreatic cancer, ovarian cancer, prostate cancer, and glioblastoma. In some embodiments, the cancer is pancreatic cancer. In some embodiments, the tumor suppressor protein is PTEN. In some embodiments, the oncogene is KRAS. In some embodiments, the individual has an aberration in a gene encoding the tumor suppressor protein. In some embodiments, the individual has an aberration in the oncogene. In some embodiments, the individual has an aberration in a gene encoding the tumor suppressor protein and the oncogene. In some embodiments, the mRNA encoding the tumor suppressor protein and the siRNA targeting the oncogene are delivered simultaneously. In some embodiments, the mRNA and the siRNA are delivered concurrently. In some embodiments, the mRNA and/or the siRNA are delivered intravenously. In some embodiments, the mRNA and/or the siRNA are complexed with a cell-penetrating peptide when delivered into the individual. In some embodiments, the cell-penetrating peptide is selected from the group consisting of CADY, PEP-1, PEP-2, MPG, VEPEP-3 peptides (used herein interchangeably with ADGN-103 peptides), VEPEP-4 peptides (used herein interchangeably with ADGN-104 peptides), VEPEP-5 peptides (used herein interchangeably with ADGN-105 peptides), VEPEP-6 peptides (used herein interchangeably with ADGN-106 peptides), VEPEP-9 peptides (used herein interchangeably with ADGN-109 peptides), and ADGN-100 peptides. In some embodiments, the cell-penetrating peptide is selected from ADGN 106 peptides and ADGN-100 peptides. In some embodiments, the mRNA (e.g., the mRNA encoding PTEN) and/or the siRNA (e.g., the siRNA targeting KRAS) are delivered about once a week or once every five days. In some embodiments, the dose of the mRNA (e.g., the mRNA encoding PTEN) for each administration in an individual is about 0.001 mg/kg to about 10 mg/kg (e.g, about 0.01 mg/kg to about 1 mg/kg, about 0.02 mg/kg to about 0.1 mg/kg). In some embodiments, the dose of the siRNA (e.g., the siRNA targeting KRAS) for each administration in an individual is about 0.001 mg/kg to about 10 mg/kg (e.g., about 0.01 mg/kg to about 1 mg/kg, about 0.02 mg/kg to about 0.1 mg/kg). In some embodiments, the dose of the mRNA (e.g., the mRNA encoding PTEN) for each administration in an individual is about 0.03 mg/m² to about 400 mg/m² (e.g., about 0.4 mg/m² to about 40 mg/m², about 0.8 mg/m² to about 4 mg/m²). In some embodiments, the dose of the siRNA (e.g., the siRNA targeting KRAS) for each administration in an individual is about 0.03 mg/m² to about 400 mg/m² (e.g., about 0.4 mg/m² to about 40 mg/m², about 0.8 mg/m² to about 4 mg/m²). In some embodiments, the individual is a human.

In some embodiments, there are also provided methods of aiding assessment of whether an individual with a disease or condition such as cancer will likely respond to or is suitable for treatment based on the individual having an aberration, wherein the treatment comprises delivering to the individual an mRNA and/or a RNAi (e.g., siRNA). In some embodiments, the aberration is in the gene corresponding to the mRNA and/or the gene targeted by the RNAi (e.g., siRNA). In some embodiments, the aberration is in the protein corresponding to the mRNA and/or the protein corresponding to the gene targeted by the RNAi (e.g., siRNA). In some embodiments, the presence of the aberration indicates that the individual will likely be responsive to the treatment, and the absence of the aberration indicates that the individual is less likely to respond to the treatment. In some embodiments, the mRNA encodes a tumor suppressor protein (e.g., PTEN or p53). In some embodiments, the RNAi (e.g., siRNA) targets an oncogene (e.g., KRAS).

In some embodiments, there is provided a method of identifying an individual with disease or condition (such as cancer) likely to respond to treatment comprising a) identifying the individual based on the individual having the aberration; and b) delivering to the individual an mRNA and/or a RNAi (e.g., siRNA). In some embodiments, the aberration is in the gene corresponding to the mRNA and/or the gene targeted by the RNAi (e.g., siRNA). In some embodiments, the aberration is in the protein corresponding to the mRNA and/or the protein corresponding to the gene targeted by the RNAi (e.g., siRNA). In some embodiments, the mRNA encodes a tumor suppressor protein (e.g., PTEN or p53). In some embodiments, the RNAi (e.g., siRNA) targets an oncogene (e.g., KRAS).

Also provided herein are methods of adjusting therapy treatment of an individual with a disease or condition (such as cancer) receiving an mRNA and/or a RNAi (e.g., siRNA); the method comprising assessing an aberration, and adjusting the therapy treatment based on the status of the aberration. In some embodiments, the aberration is in the gene corresponding to the mRNA and/or the gene targeted by the RNAi (e.g, siRNA). In some embodiments, the aberration is in the protein corresponding to the mRNA and/or the protein corresponding to the gene targeted by the RNAi (e.g., siRNA). In some embodiments, the mRNA encodes a tumor suppressor protein (e.g., PTEN or p53). In some embodiments, the RNAi (e.g., siRNA) targets an oncogene (e.g., KRAS).

Also provided herein are methods of marketing a therapy comprising an mRNA and/or a RNAi (e.g., siRNA) for use in a disease or disorder in an individual population, the method comprising informing a target audience about the use of the therapy for treating the individual population characterized by the individuals of such population having a sample which has an aberration. In some embodiments, the aberration is in the gene corresponding to the mRNA and/or the gene targeted by the RNAi (e.g, siRNA). In some embodiments, the aberration is in the protein corresponding to the mRNA and/or the protein corresponding to the gene targeted by the RNAi (e.g., siRNA). In some embodiments, the mRNA encodes a tumor suppressor protein (e.g., PTEN or p53). In some embodiments, the RNAi (e.g., siRNA) targets an oncogene (e.g., KRAS).

“Aberration” refers to a genetic aberration, an aberrant expression level and/or an aberrant activity level of the gene corresponds to the mRNA and/or the gene targeted by the siRNA that may lead to abonormal expression and/or activity of the proteins correspond to the mRNA or the gene (e.g., oncogene) targeted by the siRNA. In some embodiments, the protein with the aberration has an increased or decreased expression/activity of the protein or a signaling pathway involving the protein to a level that is above or below a reference activity level or range, such as at least about any of 10%, 20%, 30%, 40%, 60%, 70%, 80%, 90%, 100%, 200%, 500% or more above or below the reference activity level or the median of the reference activity range. In some embodiments, the reference activity level is a clinically accepted normal activity level in a standardized test, or an activity level in a healthy individual (or tissue or cell isolated from the individual) free of the aberration.

The “status” of an aberration may refer to the presence or absence of the aberration in the gene, or the aberrant level (expression or activity level, including phosphorylation level of a protein). In some embodiments, the presence of a genetic aberration (such as a mutation or a copy number variation) in the gene as compared to a control indicates that (a) the individual is more likely to respond to treatment or (b) the individual is selected for treatment. In some embodiments, the absence of a genetic aberration in a gene corresponding to the mRNA or the gene targeted by the RNAi (e.g., siRNA) compared to a control, indicates that (a) the individual is less likely to respond to treatment or (b) the individual is not selected for treatment. In some embodiments, an aberrant level (such as expression level or activity level, including phosphorylation level of a protein) of the gene corresponding to the mRNA or the gene targeted by the RNAi (e.g., siRNA) delivered or to be delivered is correlated with the likelihood of the individual to respond to treatment. For example, a larger deviation of the level (such as expression level or activity level, including phosphorylation level of a protein) of the gene corresponding to the mRNA or the gene targeted by the RNAi (e.g., siRNA) delivered or to be delivered indicates that the individual is more likely to respond to treatment. In some embodiments, a prediction model based on the level(s) (such as expression level or activity level, including phosphorylation level of a protein) of the gene corresponding to the mRNA or the gene targeted by the RNAi (e.g., siRNA) delivered or to be delivered is used to predict (a) the likelihood of the individual to respond to treatment and (b) whether to select the individual for treatment. The prediction model, including, for example, coefficient for each level, may be obtained by statistical analysis, such as regression analysis, using clinical trial data.

The expression level, and/or activity level of the protein corresponding to the mRNA or encoded by the gene targeted by the RNAi (e.g., siRNA) delivered or to be delivered, and/or the presence or absence of the gene corresponding to the mRNA or the gene targeted by the RNAi (e.g., siRNA) delivered or to be delivered can be useful for determining any of the following: (a) probable or likely suitability of an individual to initially receive treatment(s); (b) probable or likely unsuitability of an individual to initially receive treatment(s); (c) responsiveness to treatment; (d) probable or likely suitability of an individual to continue to receive treatment(s); I probable or likely unsuitability of an individual to continue to receive treatment(s); (f) adjusting dosage; (g) predicting likelihood of clinical benefits.

As used herein, “based upon” includes assessing, determining, or measuring the individual's characteristics as described herein (and preferably selecting an individual suitable for receiving treatment). When the status of an aberration is “used as a basis” for selection, assessing, measuring, or determining method of treatment as described herein, the aberration in the gene corresponding to the mRNA or the gene targeted by the RNAi (e.g., siRNA) delivered or to be delivered is determined before and/or during treatment, and the status (including presence, absence, expression level, and/or activity level of the aberration) obtained is used by a clinician in assessing any of the following: (a) probable or likely suitability of an individual to initially receive treatment(s); (b) probable or likely unsuitability of an individual to initially receive treatment(s) (c) responsiveness to treatment; (d) probable or likely suitability of an individual to continue to receive treatment(s); I probable or likely unsuitability of an individual to continue to receive treatment(s); (f) adjusting dosage; or (g) predicting likelihood of clinical benefits.

The aberration in an individual can be assessed or determined by analyzing a sample from the individual. The assessment may be based on fresh tissue samples or archived tissue samples. Suitable samples include, but are not limited to, cancer tissue, normal tissue adjacent to the cancer tissue, normal tissue distal to the cancer tissue, or peripheral blood lymphocytes. In some embodiments, the sample is a biopsy containing cancer cells, such as fine needle aspiration of cancer cells or laparoscopy obtained cancer cells. In some embodiments, the biopsied cells are centrifuged into a pellet, fixed, and embedded in paraffin prior to the analysis. In some embodiments, the biopsied cells are flash frozen prior to the analysis. In some embodiments, the sample is a plasma sample.

In some embodiments, the sample comprises a circulating metastatic cancer cell. In some embodiments, the sample is obtained by sorting circulating tumor cells (CTCs) from blood. In some further embodiments, the CTCs have detached from a primary tumor and circulate in a bodily fluid. In some further embodiments, the CTCs have detached from a primary tumor and circulate in the bloodstream. In some embodiments, the CTCs are an indication of metastasis.

The aberration may be assessed before the start of the treatment, at any time during the treatment, and/or at the end of the treatment.

I. aberration

Candidate aberrations may be identified through a variety of methods, for example, by literature search or by experimental methods known in the art, including, but not limited to, gene expression profiling experiments (e.g. RNA sequencing or microarray experiments), quantitative proteomics experiments, and gene sequencing experiments. For example, gene expression profiling experiments and quantitative proteomics experiments conducted on a sample collected from an individual having a cancer compared to a control sample may provide a list of genes and gene products (such as RNA, protein, and phosphorylated protein) that are present at aberrant levels. In some instances, gene sequencing (such as exome sequencing) experiments conducted on a sample collected from an individual having a cancer compared to a control sample may provide a list of genetic aberrations. Statistical association studies (such as genome-wide association studies) may be performed on experimental data collected from a population of individuals having a cancer to associate aberrations (such as aberrant levels or genetic aberrations) identified in the experiments with the cancer. In some embodiments, targeted sequencing experiments (such as the ONCOPANEL™ test) are conducted to provide a list of genetic aberrations in an individual having a cancer.

The ONCOPANEL™ test can be used to survey exonic DNA sequences of cancer related genes and intronic regions for detection of genetic aberrations, including somatic mutations, copy number variations and structural rearrangements in DNA from various sources of samples (such as a tumor biopsy or blood sample), thereby providing a candidate list of genetic aberrations. In some embodiments, the gene aberration is a genetic aberration or an aberrant level (such as expression level or activity level) in a gene selected from the ONCOPANEL™ test (CLIA certified). See, for example, Wagle N. et al. Cancer discovery 2.1 (2012): 82-93.

An exemplary version of ONCOPANEL™ test includes 300 cancer genes and 113 introns across 35 genes. The 300 genes included in the exemplary ONCOPANEL™ test are: ABL1, AKT1, AKT2, AKT3, ALK, ALOX12B, APC, AR, ARAF, ARID1A, ARID1B, ARID2, ASXL1, ATM, ATRX, AURKA, AURKB, AXL, B2M, BAP1, BCL2, BCL2L1, BCL2L12, BCL6, BCOR, BCORL1, BLM, BMPR1A, BRAF, BRCA1, BRCA2, BRD4, BRIP1, BUB1B, CADM2, CARD11, CBL, CBLB, CCND1, CCND2, CCND3, CCNE1, CD274, CD58, CD79B, CDC73, CDH1, CDK1, CDK2, CDK4, CDK5, CDK6, CDK9, CDKN1A, CDKN1B, CDKN1C, CDKN2A, CDKN2B, CDKN2C, CEBPA, CHEK2, CIITA, CREBBP, CRKL, CRLF2, CRTC1, CRTC2, CSF1R, CSF3R, CTNNB1, CUX1, CYLD, DDB2, DDR2, DEPDC5, DICER1, DIS3, DMD, DNMT3A, EED, EGFR, EP300, EPHA3, EPHA5, EPHA7, ERBB2, ERBB3, ERBB4, ERCC2, ERCC3, ERCC4, ERCC5, ESR1, ETV1, ETV4, ETV5, ETV6, EWSR1, EXT1, EXT2, EZH2, FAM46C, FANCA, FANCC, FANCD2, FANCE, FANCF, FANCG, FAS, FBXW7, FGFR1, FGFR2, FGFR3, FGFR4, FH, FKBP9, FLCN, FLT1, FLT3, FLT4, FUS, GATA3, GATA4, GATA6, GLI1, GLI2, GLI3, GNA11, GNAQ, GNAS, GNB2L1, GPC3, GSTM5, H3F3A, HNF1A, HRAS, ID3, IDH1, IDH2, IGF1R, IKZF1, IKZF3, INSIG1, JAK2, JAK3, KCNIP1, KDM5C, KDM6A, KDM6B, KDR, KEAP1, KIT, KRAS, LINC00894, LMO1, LMO2, LMO3, MAP2K1, MAP2K4, MAP3K1, MAPK1, MCL1, MDM2, MDM4, MECOM, MEF2B, MEN1, MET, MITF, MLH1, MLL (KMT2A), MLL2 (KTM2D), MPL, MSH2, MSH6, MTOR, MUTYH, MYB, MYBL1, MYC, MYCL1 (MYCL), MYCN, MYD88, NBN, NEGR1, NF1, NF2, NFE2L2, NFKB1A, NFKB1Z, NKX2-1, NOTCH1, NOTCH2, NPM1, NPRL2, NPRL3, NRAS, NTRK1, NTRK2, NTRK3, PALB2, PARK2, PAX5, PBRM1, PDCD1LG2, PDGFRA, PDGFRB, PHF6, PHOX2B, PIK3C2B, PIK3CA, PIK3R1, PIM, PMS1, PMS2, PNRC1, PRAME, PRDM1, PRF1, PRKAR1A, PRKCI, PRKCZ, PRKDC, PRPF40B, PRPF8, PSMD13, PTCH1, PTEN, PTK2, PTPN11, PTPRD, QKI, RAD21, RAF1, RARA, RB1, RBL2, RECQL4, REL, RET, RFWD2, RHEB, RHPN2, ROS1, RPL26, RUNX1, SBDS, SDHA, SDHAF2, SDHB, SDHC, SDHD, SETBP1, SETD2, SF1, SF3B1, SH2B3, SLITRK6, SMAD2, SMAD4, SMARCA4, SMARCB1, SMC1A, SMC3, SMO, SOCS1, SOX2, SOX9, SQSTM1, SRC, SRSF2, STAG1, STAG2, STAT3, STAT6, STK11, SUFU, SUZ12, SYK, TCF3, TCF7L1, TCF7L2, TERC, TERT, TET2, TLR4, TNFAIP3, TP53, TSC1, TSC2, U2AF1, VHL, WRN, WT1, XPA, XPC, XPO1, ZNF217, ZNF708, ZRSR2, The intronic regions surveyed in the exemplary ONCOPANEL™ test are tiled on specific introns of ABL1, AKT3, ALK, BCL2, BCL6, BRAF, CIITA, EGFR, ERG, ETV1 EWSR1, FGFR1, FGFR2, FGFR3, FUS, IGH, IGL, JAK2, MLL, MYC, NPM1, NTRK1, PAX5, PDGFRA, PDGFRB, PPARG, RAF1, RARA, RET, ROS1, SS18, TRA, TRB, TRG, TMPRSS2, aberrations (such as genetic aberration and aberrant levels) of any of the genes included in any embodiment or version of the ONCOPANEL™ test, including, but not limited to the genes and intronic regions listed above, are contemplated by the present application to serve as a basis for selecting an individual for treatment with an mRNA encoding a cancer gene as above, and/or an siRNA targeting a cancer gene as above.

### II. Genetic Aberrations

Genetic aberrations of the gene(s) corresponding to an mRNA and/or targeted by an RNAi (e.g., siRNA) delivered or to be delivered into an individual may comprise a change to the nucleic acid (such as DNA and RNA) or protein sequence (i.e. mutation) or an epigenetic feature associated with the gene, including, but not limited to, coding, non-coding, regulatory, enhancer, silencer, promoter, intron, exon, and untranslated regions of the gene(s).

The genetic aberration may be a germline mutation (including chromosomal rearrangement), or a somatic mutation (including chromosomal rearrangement). In some embodiments, the genetic aberration is present in all tissues, including normal tissue and the cancer tissue, of the individual. In some embodiments, the genetic aberration is present only in the cancer tissue of the individual. In some embodiments, the genetic aberration is present only in a fraction of the cancer tissue.

In some embodiments, the aberration comprises a mutation of the gene, including, but not limited to, deletion, frameshift, insertion, indel, missense mutation, nonsense mutation, point mutation, single nucleotide variation (SNV), silent mutation, splice site mutation, splice variant, and translocation. In some embodiments, the mutation may be a loss of function mutation for a negative regulator of the signaling pathway or a gain of function mutation of a positive regulator of the signaling pathway involving the gene.

In some embodiments, the genetic aberration comprises a copy number variation of the gene. For example, if there are normally N copies of the gene per genome. In some embodiments, the copy number of the gene is amplified by the genetic aberration, resulting in at least about any of 2, 3, 4, 5, 6, 7, 8, or more copies of the gene in the genome. In some embodiments, the genetic aberration of the gene results in loss of some copies of the gene in the genome. In some embodiments, the copy number variation of the gene is deletion of the gene. In some embodiments, the copy number variation of the gene is caused by structural rearrangement of the genome, including deletions, duplications, inversion, and translocation of a chromosome or a fragment thereof.

In some embodiments, the genetic aberration comprises an aberrant epigenetic feature associated with the gene, including, but not limited to, DNA methylation, hydroxymethylation, aberrant histone binding, chromatin remodeling, and the like. In some embodiments, the promotor of the gene is hypermethylated in the individual, for example by at least about any of 10%, 20%, 30%, 40%, 50%, 60%, 70%, 80%, 90%, or more compared to a control level (such as a clinically accepted normal level in a standardized test).

In some embodiments, there is a genetic aberration (such as a mutation or a copy number variation) in any one of the genes described above.

Genetic aberrations in genes have been identified in various human cancers, including hereditary cancers and sporadic cancers. For example, germline inactivating mutations in TSC1/2 cause tuberous sclerosis, and patients with this condition are present with lesions that include skin and brain hamartomas, renal angiomyolipomas, and renal cell carcinoma (RCC) (Krymskaya V P et al. 2011 FASEB Journal 25(6):1922-1933). PTEN hamartoma tumor syndrome (PHTS) is linked to inactivating germline PTEN mutations and is associated with a spectrum of clinical manifestations, including breast cancer, endometrial cancer, follicular thyroid cancer, hamartomas, and RCC (Legendre C. et al. 2003 Transplantation proceedings 35(3 Suppl): 151S-153S). In addition, sporadic kidney cancer has also been shown to harbor somatic mutations in several genes in the PI3K-Akt-mTOR pathway (e.g. AKT1, MTOR, PIK3CA, PTEN, RHEB, TSC1, TSC2) (Power L A, 1990 Am. J. Hosp. Pharm. 475.5: 1033-1049; Badesch D B et al. 2010 Chest 137(2): 376-3871; Kim J C & Steinberg G D, 2001, The Journal of urology, 165(3): 745-756; McKieman J. et al. 2010, J. Urol. 183 (Suppl 4)). Exemplary genetic aberrations in some genes are described below, and it is understood that the present application is not limited to the exemplary genetic aberrations described herein.

In some embodiments, the aberration comprises a genetic aberration in PTEN. In some embodiments, the genetic aberration comprises a deletion of PTEN in the genome. In some embodiments, the genetic aberration comprises a loss of function mutation in PTEN. In some embodiments, the loss of function mutation comprises a missense mutation, a nonsense mutation or a frameshift mutation. In some embodiments, the mutation comprises at a position in PTEN selected from the group consisting of K125E, K125X, E150Q, D153Y D153N K62R, Y65C, V217A, and N323K. In some embodiments, the genetic aberration comprises a loss of heterozygosity (LOH) at the PTEN locus.

In some embodiments, the aberration comprises a genetic aberration in TP53. In some embodiments, the genetic aberration comprises a loss of function mutation in TP53. In some embodiments, the loss of function mutation is a frameshift mutation in TP53, such as A39fs*5.

In some embodiments, the aberration comprises a genetic aberration in AKT. In some embodiments, the genetic aberration comprises a loss of function mutation in AKT. In some embodiments, the loss of function mutation is a frameshift mutation in AKT.

In some embodiments, the aberration comprises a genetic aberration in KRAS. In some embodiments, the genetic aberration comprises a loss of function mutation in KRAS. In some embodiments, the loss of function mutation is a frameshift mutation in KRAS.

The genetic aberrations of the genes may be assessed based on a sample, such as a sample from the individual and/or reference sample. In some embodiments, the sample is a tissue sample or nucleic acids extracted from a tissue sample. In some embodiments, the sample is a cell sample (for example a CTC sample) or nucleic acids extracted from a cell sample. In some embodiments, the sample is a tumor biopsy. In some embodiments, the sample is a tumor sample or nucleic acids extracted from a tumor sample. In some embodiments, the sample is a biopsy sample or nucleic acids extracted from the biopsy sample. In some embodiments, the sample is a Formaldehyde Fixed-Paraffin Embedded (FFPE) sample or nucleic acids extracted from the FFPE sample. In some embodiments, the sample is a blood sample. In some embodiments, cell-free DNA is isolated from the blood sample. In some embodiments, the biological sample is a plasma sample or nucleic acids extracted from the plasma sample.

The genetic aberrations of the gene may be determined by any method known in the art. See, for example, Dickson et al. Int. J. Cancer, 2013, 132(7): 1711-1717: Wagle N. Cancer Discovery, 2014, 4:546-553; and Cancer Genome Atlas Research Network. Nature 2013, 499: 43-49. Exemplary methods include, but are not limited to, genomic DNA sequencing, bisulfite sequencing or other DNA sequencing-based methods using Sanger sequencing or next generation sequencing platforms; polymerase chain reaction assays; in situ hybridization assays; and DNA microarrays. The epigenetic features (such as DNA methylation, histone binding, or chromatin modifications) of one or more genes from a sample isolated from the individual may be compared with the epigenetic features of the one or more genes from a control sample. The nucleic acid molecules extracted from the sample can be sequenced or analyzed for the presence of the genetic aberrations relative to a reference sequence, such as the wildtype sequences of TP53, KRAS. AKT, and PTEN.

In some embodiments, the genetic aberration of a gene is assessed using cell-free DNA sequencing methods. In some embodiments, the genetic aberration of a gene is assessed using next-generation sequencing. In some embodiments, the genetic aberration of a gene isolated from a blood sample is assessed using next-generation sequencing. In some embodiments, the genetic aberration of a gene is assessed using exome sequencing. In some embodiments, the genetic aberration of a gene is assessed using fluorescence in-situ hybridization analysis. In some embodiments, the genetic aberration of a gene is assessed prior to initiation of the methods of treatment described herein. In some embodiments, the genetic aberration of a gene is assessed after initiation of the methods of treatment described herein. In some embodiments, the genetic aberration of a gene is assessed prior to and after initiation of the methods of treatment described herein. An aberrant level of a gene may refer to an aberrant expression level or an aberrant activity level.

III. Aberrant levels

Aberrant expression level of a gene comprises an increase or decrease in the level of a molecule encoded by the gene compared to the control level. The molecule encoded by the gene may include RNA transcript(s) (such as mRNA), protein isoform(s), phosphorylated and/or dephosphorylated states of the protein isoform(s), ubiquitinated and/or de-ubiquitinated states of the protein isoform(s), membrane localized (e.g. myristoylated, palmitoylated, and the like) states of the protein isoform(s), other post-translationally modified states of the protein isoform(s), or any combination thereof.

Aberrant activity level of a gene comprises enhancement or repression of a molecule encoded by any downstream target gene of the gene, including epigenetic regulation, transcriptional regulation, translational regulation, post-translational regulation, or any combination thereof of the downstream target gene. Additionally, activity of a gene comprises downstream cellular and/or physiological effects in response to the aberration, including, but not limited to, protein synthesis, cell growth, proliferation, signal transduction, mitochondria metabolism, mitochondria biogenesis, stress response, cell cycle arrest, autophagy, microtubule organization, and lipid metabolism.

In some embodiments, the aberration (e.g aberrant expression level) comprises an aberrant protein phosphorylation level. In some embodiments, the aberrant phosphorylation level is in a protein encoded by a gene selected from the group consisting of PTEN, KRAS, AKT, and TP53. Exemplary phosphorylated species of genes may serve as relevant biomarkers. In some embodiments, the individual is selected for treatment if the protein in the individual is phosphorylated. In some embodiments, the individual is selected for treatment if the protein in the individual is not phosphorylated. In some embodiments, the phosphorylation status of the protein is determined by immunohistochemistry.

The levels (such as expression levels and/or activity levels) of a gene in an individual may be determined based on a sample (e.g., sample from the individual or reference sample). In some embodiments, the sample is from a tissue, organ, cell, or tumor. In some embodiments, the sample is a biological sample. In some embodiments, the biological sample is a biological fluid sample or a biological tissue sample. In further embodiments, the biological fluid sample is a bodily fluid. In some embodiments, the sample is a cancer tissue, normal tissue adjacent to said cancer tissue, normal tissue distal to said cancer tissue, blood sample, or other biological sample. In some embodiments, the sample is a fixed sample. Fixed samples include, but are not limited to, a formalin fixed sample, a paraffin-embedded sample, or a frozen sample. In some embodiments, the sample is a biopsy containing cancer cells. In a further embodiment, the biopsy is a fine needle aspiration of cancer cells. In a further embodiment, the biopsy is laparoscopy obtained cancer cells. In some embodiments, the biopsied cells are centrifuged into a pellet, fixed, and embedded in paraffin. In some embodiments, the biopsied cells are flash frozen. In some embodiments, the biopsied cells are mixed with an antibody that recognizes a molecule encoded by the gene. In some embodiments, the at least one gene comprises enhancement or repression of a molecule encoded by any downstream target gene of the gene, including epigenetic regulation, transcriptional regulation, translational regulation, post-translational regulation, or any combination thereof of the downstream target gene. Additionally, activity of a gene comprises downstream cellular and/or physiological effects in response to the aberration, including, but not limited to, protein synthesis, cell growth, proliferation, signal transduction, mitochondria metabolism, mitochondria biogenesis, stress response, cell cycle arrest, autophagy, microtubule organization, and lipid metabolism.

In some embodiments, the sample comprises a circulating metastatic cancer cell. In some embodiments, the sample is obtained by sorting circulating tumor cells (CTCs) from blood. In a further embodiment, the CTCs have detached from a primary tumor and circulate in a bodily fluid. In yet a further embodiment, the CTCs have detached from a primary tumor and circulate in the bloodstream. In a further embodiment, the CTCs are an indication of metastasis.

In some embodiments, the level of a protein encoded by a gene is determined to assess the aberrant expression level of the gene. In some embodiments, the level of a protein encoded by a downstream target gene of a gene is determined to assess the aberrant activity level of the gene. In some embodiments, protein level is determined using one or more antibodies specific for one or more epitopes of the individual protein or proteolytic fragments thereof. Detection methodologies suitable for use in the practice of the invention include, but are not limited to, immunohistochemistry, enzyme linked immunosorbent assays (ELISAs), Western blotting, mass spectroscopy, and immuno-PCR. In some embodiments, levels of protein(s) encoded by the gene and/or downstream target gene(s) thereof in a sample are normalized (such as divided) by the level of a housekeeping protein (such as glyceraldehyde 3-phosphate dehydrogenase, or GAPDH) in the same sample.

In some embodiments, the level of an mRNA encoded by a gene is determined to assess the aberrant expression level of the gene. In some embodiments, the level of an mRNA encoded by a downstream target gene of a gene is determined to assess the aberrant activity level of the gene. In some embodiments, a reverse-transcription (RT) polymerase chain reaction (PCR) assay (including a quantitative RT-PCR assay) is used to determine the mRNA levels. In some embodiments, a gene chip or next-generation sequencing methods (such as RNA (cDNA) sequencing or exome sequencing) are used to determine the levels of RNA (such as mRNA) encoded by the gene and/or downstream target genes thereof. In some embodiments, an mRNA level of the gene and/or downstream target genes thereof in a sample are normalized (such as divided) by the mRNA level of a housekeeping gene (such as GAPDH) in the same sample.

In some embodiments, the level of the gene in an individual is compared to the level of the gene in a control sample. In some embodiments, the level of the gene in an individual is compared to the level of the gene in multiple control samples. In some embodiments, multiple control samples are used to generate a statistic that is used to classify the level of the gene in an individual with a cancer.

The classification or ranking of the level (i.e., high or low) of the gene may be determined relative to a statistical distribution of control levels. In some embodiments, the classification or ranking is relative to a control sample, such as a normal tissue (e.g. peripheral blood mononuclear cells), or a normal epithelial cell sample (e.g. a buccal swap or a skin punch) obtained from the individual. In some embodiments, the level of the gene is classified or ranked relative to a statistical distribution of control levels. In some embodiments, the level of the gene is classified or ranked relative to the level from a control sample obtained from the individual.

Control samples can be obtained using the same sources and methods as non-control samples. In some embodiments, the control sample is obtained from a different individual (for example an individual not having the cancer; an individual having a benign or less advanced form of a disease corresponding to the cancer; and/or an individual sharing similar ethnic, age, and gender). In some embodiments when the sample is a tumor tissue sample, the control sample may be a non-cancerous sample from the same individual. In some embodiments, multiple control samples (for example from different individuals) are used to determine a range of levels of the genes in a particular tissue, organ, or cell population.

In some embodiments, the control sample is a cultured tissue or cell that has been determined to be a proper control. In some embodiments, the control is a cell that does not have the aberration. In some embodiments, a clinically accepted normal level in a standardized test is used as a control level for determining the aberrant level of the gene. In some embodiments, the level of the gene or downstream target genes thereof in the individual is classified as high, medium or low according to a scoring system, such as an immunohistochemistry-based scoring system.

In some embodiments, the level of the gene is determined by measuring the level of the gene in an individual and comparing to a control or reference (e.g., the median level for the given patient population or level of a second individual). For example, if the level of the gene for the single individual is determined to be above the median level of the patient population, that individual is determined to have high expression level of the gene. Alternatively, if the level of the gene for the single individual is determined to be below the median level of the patient population, that individual is determined to have low expression level of the gene. In some embodiments, the individual is compared to a second individual and/or a patient population which is responsive to the treatment. In some embodiments, the individual is compared to a second individual and/or a patient population which is not responsive to the treatment. In some embodiments, the levels are determined by measuring the level of a nucleic acid encoded by the gene and/or a downstream target gene thereof. For example, if the level of a molecule (such as an mRNA or a protein) encoded by the gene for the single individual is determined to be above the median level of the patient population, that individual is determined to have a high level of the molecule (such as mRNA or protein) encoded by the gene. Alternatively, if the level of a molecule (such as an mRNA or a protein) encoded by the gene for the single individual is determined to be below the median level of the patient population, that individual is determined to have a low level of the molecule (such as mRNA or protein) encoded by the gene.

In some embodiments, the control level of a gene is determined by obtaining a statistical distribution of the levels of gene. In some embodiments, the level of the gene is classified or ranked relative to control levels or a statistical distribution of control levels.

In some embodiments, bioinformatics methods are used for the determination and classification of the levels of the gene, including the levels of downstream target genes of the gene as a measure of the activity level of the gene. Numerous bioinformatics approaches have been developed to assess gene set expression profiles using gene expression profiling data. Methods include but are not limited to those described in Segal, E. et a. Nat. Genet. 34:66-176 (2003); Segal, E. et al. Nat. Genet. 36:1090-1098 (2004): Barry, W. T. et al. Bioinformatics 21:1943-1949 (2005); Tian, L. et al. Proc Nat'l Acad Sci USA 102:13544-13549 (2005): Novak B A and Jain A N. Bioinformatics 22:233-41 (2006); Maglietta R et al. Bioinformatics 23:2063-72 (2007); Bussemaker H J, BMC Bioinformatics 8 Suppl 6:S6 (2007).

In some embodiments, the control level is a pre-determined threshold level. In some embodiments, mRNA level is determined, and a low level is an mRNA level less than about any of 1, 0.9, 0.8, 0.7, 0.6, 0.5, 0.4, 0.3, 0.2, 0.1, 0.05, 0.02, 0.01, 0.005, 0.002, 0.001 or less time that of what is considered as clinically normal or of the level obtained from a control. In some embodiments, a high level is an mRNA level more than about 1.1, 1.2, 1.3, 1.5, 1.7, 2, 2.2, 2.5, 2.7, 3, 5, 7, 10, 20, 50, 70, 100, 200, 500, 1000 times or more than 1000 times that of what is considered as clinically normal or of the level obtained from a control.

In some embodiments, protein expression level is determined, for example by Western blot or an enzyme-linked immunosorbent assay (ELISA). For example, the criteria for low or high levels can be made based on the total intensity of a band on a protein gel corresponding to the protein encoded by the gene that is blotted by an antibody that specifically recognizes the protein encoded by the gene, and normalized (such as divided) by a band on the same protein gel of the same sample corresponding to a housekeeping protein (such as GAPDH) that is blotted by an antibody that specifically recognizes the housekeeping protein (such as GAPDH). In some embodiments, the protein level is low if the protein level is less than about any of 1, 0.9, 0.8, 0.7, 0.6, 0.5, 0.4, 0.3, 0.2, 0.1, 0.05, 0.02, 0.01, 0.005, 0.002, 0.00 or less time of what is considered as clinically normal or of the level obtained from a control. In some embodiments, the protein level is high if the protein level is more than about any of 1.1, 1.2, 1.3, 1.5, 1.7, 2, 2.2, 2.5, 2.7, 3, 5, 7, 10, 20, 50, or 100 times or more than 100 times of what is considered as clinically normal or of the level obtained from a control.

In some embodiments, protein expression level is determined, for example by immunohistochemistry. For example, the criteria for low or high levels can be made based on the number of positive staining cells and/or the intensity of the staining, for example by using an antibody that specifically recognizes the protein encoded by the gene. In some embodiments, the level is low if less than about 1%, 5%, 10%, 15%, 20%, 25%, 30%, 35%, 40%, 45%, or 50% cells have positive staining. In some embodiments, the level is low if the staining is 1%, 5%, 10%, 15%, 20%, 25%, 30%, 35%, 40%, 45%, or 50% less intense than a positive control staining. In some embodiments, the level is high if more than about 40%, 45%, 50%, 55%, 60%, 65%, 70%, 75%, 80%, 85%, or 90%, cells have positive staining. In some embodiments, the level is high if the staining is as intense as positive control staining. In some embodiments, the level is high if the staining is 80%, 85%, or 90% as intense as positive control staining.

In some embodiments, the scoring is based on an “H-score” as described in US Pat. Pub. No. 2013/0005678. An H-score is obtained by the formula: 3× percentage of strongly staining cells+2× percentage of moderately staining cells+percentage of weakly staining cells, giving a range of 0 to 300.

In some embodiments, strong staining, moderate staining, and weak staining are calibrated levels of staining, wherein a range is established and the intensity of staining is binned within the range. In some embodiments, strong staining is staining above the 75^{th} percentile of the intensity range, moderate staining is staining from the 25^{th} to the 75^{th} percentile of the intensity range, and low staining is staining is staining below the 25^{h} percentile of the intensity range. In some aspects one skilled in the art, and familiar with a particular staining technique, adjusts the bin size and defines the staining categories.

In some embodiments, the label high staining is assigned where greater than 50% of the cells stained exhibited strong reactivity, the label no staining is assigned where no staining was observed in less than 50% of the cells stained, and the label low staining is assigned for all of other cases.

In some embodiments, the assessment and/or scoring of the genetic aberration or the level of the gene in a sample, patient, etc., is performed by one or more experienced clinicians, i.e., those who are experienced with the gene expression and the gene product staining patterns. For example, in some embodiments, the clinician(s) is blinded to clinical characteristics and outcome for the samples, patients, etc. being assessed and scored.

In some embodiments, level of protein phosphorylation is determined. The phosphorylation status of a protein may be assessed from a variety of sample sources. In some embodiments, the sample is a tumor biopsy. The phosphorylation status of a protein may be assessed via a variety of methods. In some embodiments, the phosphorylation status is assessed using immunohistochemistry. The phosphorylation status of a protein may be site specific. The phosphorylation status of a protein may be compared to a control sample. In some embodiments, the phosphorylation status is assessed prior to initiation of the methods of treatment described herein. In some embodiments, the phosphorylation status is assessed after initiation of the methods of treatment described herein. In some embodiments, the phosphorylation status is assessed prior to and after initiation of the methods of treatment described herein.

### Kits

Also provided herein are kits, reagents, and articles of manufacture useful for the methods described herein. In some embodiments, kit contains vials containing the CPPs, assembly molecules and/or other cell-penetrating peptides, separately from vials containing the one or more mRNA. At the time of patient treatment, it is first determined what particular pathology is to be treated based on for example, gene expression analysis or proteomic or histological analysis of patient samples. Having obtained those results, the CPPs and any optional assembly molecules and/or cell-penetrating peptides are combined accordingly with the appropriate one or more mRNA to result in complexes or nanoparticles that can be administered to the patient for an effective treatment. Thus, in some embodiments, there is provided a kit comprising: 1) a CPP, and optionally 2) one or more mRNA. In some embodiments, the kit further comprises assembly molecules and/or other cell-penetrating peptides. In some embodiments, the kit further comprises agents for determining gene expression profiles. In some embodiment, the kit further comprises a pharmaceutically acceptable carrier.

In some embodiments, a kit described herein comprises a) an mRNA (e.g., mRNA encoding a tumor suppressor protein, e.g., PTEN and/or p53), and b) an RNAi (e.g., siRNA, e.g., siRNA targeting an oncogene, e.g., siRNA targeting KRAS. In some embodiments, the kit further comprises an agent to assess an aberration in an individual. In some embodiments, the aberration comprises a mutation of the gene. In some embodiments, the gene is PTEN and/or KRAS.

The kits described herein may further comprise instructions for using the components of the kit to practice the subject methods (for example instructions for making the pharmaceutical compositions described herein and/or for use of the pharmaceutical compositions). The instructions for practicing the subject methods are generally recorded on a suitable recording medium. For example, the instructions may be printed on a substrate, such as paper or plastic, etc. As such, the instructions may be present in the kits as a package insert, in the labeling of the container of the kits or components thereof (i.e., associated with the packaging or sub packaging) etc. In some embodiments, the instructions are present as an electronic storage data file present on a suitable computer readable storage medium, e.g., CD-ROM, diskette, etc. In yet other embodiments, the actual instructions are not present in the kit, but means for obtaining the instructions from a remote source, e.g., via the internet, are provided. An example of this embodiment is a kit that includes a web address where the instructions can be viewed and/or from which the instructions can be downloaded. As with the instructions, this means for obtaining the instructions is recorded on a suitable substrate

The various components of the kit may be in separate containers, where the containers may be contained within a single housing, e.g., a box.

### EXEMPLARY EMBODIMENTS

### Embodiment 1

An mRNA delivery complex for intracellular delivery of an mRNA comprising a cell-penetrating peptide (CPP) and the mRNA, wherein the cell-penetrating peptide is selected from the group consisting of VEPEP-3 peptides, VEPEP-6 peptides, VEPEP-9 peptides, and ADGN-100 peptides.

### Embodiment 2

An mRNA delivery complex for intracellular delivery of an mRNA comprising a cell-penetrating peptide (CPP) and the mRNA prepared by a process comprising the steps of: a) mixing a first solution comprising the mRNA with a second solution comprising the CPP to form a third solution, wherein the third solution comprises or is adjusted to comprise i) about 0-5% sucrose, ii) about 0-5% glucose, iii) about 0-50% DMEM, iv) about 0-80 mM NaCl, or v) about 0-20% PBS; and b) incubating the third solution to allow formation of the mRNA delivery complex.

### Embodiment 3

The mRNA delivery complex of embodiment 2, wherein the first solution comprises the mRNA in sterile water and/or wherein the second solution comprises the CPP in sterile water.

### Embodiment 4

The mRNA delivery complex of embodiment of 2 or 3, wherein the third solution is adjusted to comprise i) about 0-5% sucrose, ii) about 0-5% glucose, iii) about 0-50% DMEM, iv) about 0-80 mM NaCl, or v) about 0-20% PBS after the incubating of step b).

### Embodiment 5

An mRNA delivery complex for intracellular delivery of an mRNA comprising a cell-penetrating peptide (CPP) and the mRNA, wherein the mRNA encodes a therapeutic protein.

### Embodiment 6

The mRNA delivery complex of embodiment 5, wherein the therapeutic protein replaces a protein that is deficient or abnormal, augments an existing pathway, provides a novel function or activity, or interferes with a molecule or organism.

### Embodiment 7

An mRNA delivery complex for intracellular delivery of an mRNA comprising a cell-penetrating peptide (CPP) and the mRNA, wherein the mRNA delivery complex further comprises an RNAi.

### Embodiment 8

The mRNA delivery complex of embodiment 7, wherein the RNAi is an siRNA, an shRNA, or an miRNA.

### Embodiment 9

The mRNA delivery complex of embodiment 7 or 8, wherein the mRNA encodes a therapeutic protein for treating a disease or condition, and wherein the RNAi targets an RNA, wherein expression of the RNA is associated with the disease or condition.

### Embodiment 10

The mRNA delivery complex of any one of embodiments 1-9, wherein the cell-penetrating peptide is a VEPEP-3 peptide.

### Embodiment 11

The mRNA delivery complex of embodiment 10, wherein the cell-penetrating peptide comprises an amino acid sequence selected from the group consisting of SEQ ID NOs: 1-14.

### Embodiment 12

The mRNA delivery complex of embodiment 10, wherein the cell-penetrating peptide comprises the amino acid sequence of SEQ ID NO: 75 or 76.

### Embodiment 13

The mRNA delivery complex of any one of embodiments 1-9, wherein the cell-penetrating peptide is a VEPEP-6 peptide.

### Embodiment 14

The mRNA delivery complex of embodiment 13, wherein the cell-penetrating peptide comprises an amino acid sequence selected from the group consisting of SEQ ID NOs: 15-40.

### Embodiment 15

The mRNA delivery complex of embodiment 13, wherein the cell-penetrating peptide comprises the amino acid sequence of SEQ ID NO: 77.

### Embodiment 16

The mRNA delivery complex of any one of embodiments 1-9, wherein the cell-penetrating peptide is a VEPEP-9 peptide.

### Embodiment 17

The mRNA delivery complex of embodiment 16, wherein the cell-penetrating peptide comprises an amino acid sequence selected from the group consisting of SEQ ID NOs: 41-52.

### Embodiment 18

The mRNA delivery complex of embodiment 16, wherein the cell-penetrating peptide comprises the amino acid sequence of SEQ ID NO: 78.

### Embodiment 19

The mRNA delivery complex of any one of embodiments 1-9, wherein the cell-penetrating peptide is an ADGN-100 peptide.

### Embodiment 20

The mRNA delivery complex of embodiment 19, wherein the cell-penetrating peptide comprises an amino acid sequence selected from the group consisting of SEQ ID NOs: 53-70.

### Embodiment 21

The mRNA delivery complex of embodiment 19, wherein the cell-penetrating peptide comprises the amino acid sequence of SEQ ID NO: 79 or 80.

### Embodiment 22

The mRNA delivery complex of any one of embodiments 1-21, wherein the cell-penetrating peptide is covalently linked to the mRNA.

### Embodiment 23

The mRNA delivery complex of any one of embodiments 1-22, wherein the cell-penetrating peptide further comprises one or more moieties covalently linked to the N-terminus of the cell-penetrating peptide, and wherein the one or more moieties are selected from the group consisting of an acetyl, a fatty acid, a cholesterol, a poly-ethylene glycol, a nuclear localization signal, nuclear export signal, an antibody or fragment thereof, a polysaccharide and a targeting molecule.

### Embodiment 24

The mRNA delivery complex of embodiment 23, wherein the cell-penetrating peptide comprises an acetyl group covalently linked to its N-terminus.

### Embodiment 25

The mRNA delivery complex of any one of embodiments 1-24, wherein the cell-penetrating peptide further comprises one or more moieties covalently linked to the C-terminus of the cell-penetrating peptide, and wherein the one or more moieties are selected from the group consisting of a cysteamide, a cysteine, a thiol, an amide, a nitrilotriacetic acid optionally substituted, a carboxyl, a linear or ramified C₁-C₆ alkyl optionally substituted, a primary or secondary amine, an osidic derivative, a lipid, a phospholipid, a fatty acid, a cholesterol, a poly-ethylene glycol, a nuclear localization signal, nuclear export signal, an antibody or fragment thereof, a polysaccharide and a targeting molecule.

### Embodiment 26

The mRNA delivery complex of embodiment 25, wherein the cell-penetrating peptide comprises a cysteamide group covalently linked to its C-terminus.

### Embodiment 27

The mRNA delivery complex of any one of embodiments 1-26, wherein at least some of the cell-penetrating peptides in the mRNA delivery complex are linked to a targeting moiety by a linkage.

### Embodiment 28

The mRNA delivery complex of embodiment 27, wherein the linkage is covalent.

### Embodiment 29

The mRNA delivery complex of any one of embodiments 1-28, wherein the mRNA encodes a therapeutic protein.

### Embodiment 30

The mRNA delivery complex of embodiment 29, wherein the mRNA encodes a tumor suppressor protein.

### Embodiment 31

The mRNA delivery complex of any one of embodiments 1-30, wherein the mRNA delivery complex further comprises an RNAi.

### Embodiment 32

The mRNA delivery complex of embodiment 31, wherein the RNAi targets an oncogene for downregulation.

### Embodiment 33

The mRNA delivery complex of any one of embodiments 1-32, wherein the molar ratio of the cell-penetrating peptide to the mRNA is between about 1:1 and about 100:1.

### Embodiment 34

The mRNA delivery complex of any one of embodiments 1-33, wherein the average diameter of the mRNA delivery complex is between about 20 nm and about 1000 nm.

### Embodiment 35

A nanoparticle comprising a core comprising the mRNA delivery complex of any one of embodiments 1-34.

### Embodiment 36

The nanoparticle of embodiment 35, wherein the core further comprises one or more additional mRNA delivery complexes according to any one of embodiments 1-34.

### Embodiment 37

The nanoparticle of embodiment 35 or 36, wherein the core further comprises an RNAi.

### Embodiment 38

The nanoparticle of embodiment 37, wherein the RNAi targets an oncogene for downregulation.

### Embodiment 39

The nanoparticle of embodiment 37 or 38, wherein the RNAi is in a complex comprising a cell-penetrating peptide (CPP) and the RNAi.

### Embodiment 40

The nanoparticle of embodiment 39, wherein the cell-penetrating peptide is selected from the group consisting of PEP-1 peptides, PEP-2 peptides, PEP-3 peptides. VEPEP-3 peptides, VEPEP-6 peptides, VEPEP-9 peptides, and ADGN-100 peptides.

### Embodiment 41

The nanoparticle of any one of embodiments 35-40, wherein at least some of the cell-penetrating peptides in the nanoparticle are linked to a targeting moiety by a linkage.

### Embodiment 42

The nanoparticle of any one of embodiments 35-41, wherein the core is coated by a shell comprising a peripheral cell-penetrating peptide.

### Embodiment 43

The nanoparticle of embodiment 42, wherein the peripheral cell-penetrating peptide is selected from the group consisting of PEP-1 peptides, PEP-2 peptides, PEP-3 peptides, VEPEP-3 peptides, VEPEP-6 peptides, VEPEP-9 peptides, and ADGN-100 peptides.

### Embodiment 44

The nanoparticle of embodiment 43, wherein the peripheral cell-penetrating peptide comprises an amino acid sequence selected from the group consisting of SEQ ID NOs: 1-80.

### Embodiment 45

The nanoparticle of any one of embodiments 42-44, wherein at least some of the peripheral cell-penetrating peptides in the shell are linked to a targeting moiety by a linkage.

### Embodiment 46

The nanoparticle of embodiment 41 or 45, wherein the linkage is covalent.

### Embodiment 47

The nanoparticle of any one of embodiments 35-46, wherein the average diameter of the nanoparticle is between about 20 nm and about 1000 nm.

### Embodiment 48

A pharmaceutical composition comprising the mRNA delivery complex of any one of embodiments 1-34 or the nanoparticle of any one of embodiments 3547, and a pharmaceutically acceptable carrier.

### Embodiment 49

The pharmaceutical composition of embodiment 48, wherein the mRNA delivery complex or nanoparticle comprises an mRNA encoding a therapeutic protein.

### Embodiment 50

The pharmaceutical composition of embodiment 48 or 49, further comprising an inhibitory RNA (RNAi).

### Embodiment 51

The pharmaceutical composition of embodiment 50, wherein the RNAi is in the mRNA delivery complex or nanoparticle.

### Embodiment 52

The pharmaceutical composition of embodiment 48, wherein the mRNA delivery complex or nanoparticle comprises an mRNA encoding a chimeric antigen receptor (CAR).

### Embodiment 53

A method of preparing the mRNA delivery complex of any one of embodiments 1-34, comprising combining the cell-penetrating peptide with the one or more mRNA, thereby forming the mRNA delivery complex.

### Embodiment 54

The method of embodiment 53, wherein the cell-penetrating peptide and the mRNA are combined at a molar ratio from about 1:1 to about 100:1, respectively.

### Embodiment 55

The method of embodiment 53 or 54, wherein the combining comprises mixing a first solution comprising the mRNA with a second solution comprising the CPP to form a third solution, wherein the third solution comprises or is adjusted to comprise i) about 0-5% sucrose, ii) about 0-5% glucose, iii) about 0-50% DMEM, iv) about 0-80 mM NaCl, or v) about 0-20% PBS, and wherein the third solution is incubated to allow formation of the mRNA delivery complex.

### Embodiment 56

The method of embodiment 55, wherein the first solution comprises the mRNA in sterile water and/or wherein the second solution comprises the CPP in sterile water.

### Embodiment 57

The method of embodiment of 55 or 56, wherein the third solution is adjusted to comprise i) about 0-5% sucrose, ii) about 0-5% glucose, iii) about 0-50% DMEM, iv) about 0-80 mM NaCl, or v) about 0-20% PBS after incubating to form the mRNA delivery complex.

### Embodiment 58

A method of delivering one or more mRNA into a cell, comprising contacting the cell with the mRNA delivery complex of any one of embodiments 1-34 or the nanoparticle of any one of embodiments 35-47, wherein the mRNA delivery complex or the nanoparticle comprises the one or more mRNA.

### Embodiment 59

The method of embodiment 58, wherein the contacting of the cell with the mRNA delivery complex or nanoparticle is carried out in vivo.

### Embodiment 60

The method of embodiment 58, wherein the contacting of the cell with the mRNA delivery complex or nanoparticle is carried out ex vivo.

### Embodiment 61

The method of embodiment 58, wherein the contacting of the cell with the mRNA delivery complex or nanoparticle is carried out in vitro.

### Embodiment 62

The method of any one of embodiments 58-61, wherein the cell is a stem cell, a hematopoietic precursor cell, a granulocyte, a mast cell, a monocyte, a dendritic cell, a B cell, a T cell, a natural killer cell, a fibroblast, a muscle cell, a cardiac cell, a hepatocyte, a lung progenitor cell, or a neuronal cell.

### Embodiment 63

The method of embodiment 62, wherein the cell is a T cell.

### Embodiment 64

The method of embodiment 62 or 63, wherein the mRNA encodes a protein that is capable of modulating an immune response in an individual in which it is expressed.

### Embodiment 65

The method of any one of embodiments 58-64, wherein the mRNA delivery complex or nanoparticle comprises an mRNA encoding a therapeutic protein.

### Embodiment 66

The method of any one of embodiments 58-65, wherein the mRNA delivery complex or nanoparticle further comprises an inhibitory RNA (RNAi).

### Embodiment 67

The method of any one of embodiments 58-65, further comprising delivering an RNAi into the cell.

### Embodiment 68

The method of any one of embodiments 58-64, wherein the mRNA delivery complex or nanoparticle comprises an mRNA encoding a chimeric antigen receptor (CAR).

### Embodiment 69

A method of treating a disease in an individual comprising administering to the individual an effective amount of the pharmaceutical composition of any one of embodiments 48-52.

### Embodiment 70

The method of embodiment 69, wherein the pharmaceutical composition is administered via intravenous, intratumoral, intraarterial, topical, intraocular, ophthalmic, intraportal, intracranial, intracerebral, intracerebroventricular, intrathecal, intravesicular, intradermal, subcutaneous, intramuscular, intranasal, intratracheal, pulmonary, intracavity, or oral administration.

### Embodiment 71

The method of embodiment 69, wherein the pharmaceutical composition is administered via injection into a blood vessel wall or tissue surrounding the blood vessel wall.

### Embodiment 72

The method of embodiment 71, wherein the injection is through a catheter with a needle.

### Embodiment 73

The method of embodiment 69, wherein the disease is selected from the group consisting of cancer, diabetes, autoimmune diseases, hematological diseases, cardiac diseases, vascular diseases, inflammatory diseases, fibrotic diseases, viral infectious diseases, hereditary diseases, ocular diseases, liver diseases, lung diseases, muscle diseases, protein deficiency diseases, lysosomal storage diseases, neurological diseases, kidney diseases, aging and degenerative diseases, and diseases characterized by cholesterol level abnormality.

### Embodiment 74

The method of embodiment 69, wherein the disease is a protein deficiency disease.

### Embodiment 75

The method of embodiment 74, wherein the pharmaceutical composition comprises an mRNA delivery complex or nanoparticle comprising one or more mRNA encoding a deficient protein contributing to the disease.

### Embodiment 76

The method of embodiment 69, wherein the disease is characterized by an abnormal protein.

### Embodiment 77

The method of embodiment 76, wherein the pharmaceutical composition comprises an mRNA delivery complex or nanoparticle comprising one or more mRNA encoding a functional variant of the non-functional protein contributing to the disease.

### Embodiment 78

The method of embodiment 73, wherein the disease is cancer.

### Embodiment 79

The method of embodiment 78, wherein the cancer is a solid tumor, and wherein the pharmaceutical composition comprises an mRNA delivery complex or nanoparticle comprising one or more mRNA encoding a tumor suppressor protein useful for treating the solid tumor.

### Embodiment 80

The method of embodiment 79, wherein the cancer is cancer of the liver, lung, kidney, colorectum, or pancreas.

### Embodiment 81

The method of embodiment 78, wherein the cancer is a hematological malignancy, and wherein the pharmaceutical composition comprises an mRNA delivery complex or nanoparticle comprising one or more mRNA encoding a tumor suppressor protein useful for treating the hematological malignancy.

### Embodiment 82

The method of any one of embodiments 78-81, wherein the pharmaceutical composition further comprises an RNAi that targets an oncogene involved in the cancer development and/or progression.

### Embodiment 83

The method of embodiment 82, wherein the RNAi is in the mRNA delivery complex or nanoparticle.

### Embodiment 84

The method of embodiment 73, wherein the disease is a viral infection disease, and wherein the pharmaceutical composition comprises an mRNA delivery complex or nanoparticle comprising one or more mRNA encoding a protein involved in the viral infectious disease development and/or progression.

### Embodiment 85

The method of embodiment 73, wherein the disease is a hereditary disease, and wherein the pharmaceutical composition comprises an mRNA delivery complex or nanoparticle comprising one or more mRNA encoding one or more proteins involved in the hereditary disease development and/or progression.

### Embodiment 86

The method of embodiment 73, wherein the disease is an aging or degenerative disease, and wherein the pharmaceutical composition comprises an mRNA delivery complex or nanoparticle comprising one or more mRNA encoding one or more proteins involved in the aging or degenerative disease development and/or progression.

### Embodiment 87

The method of embodiment 73, wherein the disease is a fibrotic or inflammatory disease, and wherein the pharmaceutical composition comprises an mRNA delivery complex or nanoparticle comprising one or more mRNA encoding one or more proteins involved in the fibrotic or inflammatory disease development and/or progression.

### Embodiment 88

The method of any one of embodiments 69-87, wherein the individual is human.

### Embodiment 89

A kit comprising a composition comprising the mRNA delivery complex of any one of embodiments 1-34 and/or the nanoparticle of any one of embodiments 35-47.

### Embodiment 90

A method of treating a cancer in an individual comprising administering to the individual an effective amount of an mRNA encoding a tumor suppressor protein, wherein the tumor suppressor protein corresponds to a tumor suppressor gene selected from PTEN, Retinoblastoma RB (or RB1), TP53, TP63, TP73, CDKN2A (INK4A), CDKN1B, CDKN1C, DLD/NP1, HEPACAM, SDHB, SDHD, SFRP1, TCF21 TIG1, MLH1, MSH2, MSH6, WT1, WT2, NF1, NF2N, VHL, KLF4, pVHL, APC, CD95, ST5, YPEL3, ST7. APC, MADR2, BRCA1, BRCA2, Patched, TSC1, TSC2, PALB2, ST14, or VHL.

### Embodiment 91

The method of embodiment 90, further comprising administering to the individual an effective amount of an siRNA targeting an oncogene.

### Embodiment 92

The method of embodiment 91, wherein the oncogene comprises KRAS.

### Embodiment 93

The method of embodiment 92, wherein the siRNA targets a mutant form of KRAS, wherein the mutant form of KRAS comprises a mutation on codon 12 or 61 of KRAS.

### Embodiment 94

The method of embodiment 93, wherein the mutant form of KRAS comprises G12D KRAS.

### Embodiment 95

The method of embodiment 93 or 94, wherein the siRNA comprises a first siRNA targeting a first mutant form of KRAS, and a second siRNA targeting a second mutant form of KRAS, wherein the first mutant form of KRAS comprises G12D KRAS, and wherein the second mutant form of KRAS comprises G12C KRAS.

### Embodiment 96

The method of any one of embodiments 91-95, wherein the siRNA comprises a nucleic acid sequence selected from sequences with SEQ ID NOS: 83, 84, 86-89.

### Embodiment 97

The method of any one of embodiments 90-96, wherein the tumor suppressor gene is selected from PTEN and TP53.

### Embodiment 98

The method of embodiment 97, wherein the tumor suppressor gene is PTEN.

### Embodiment 99

The method of any one of embodiment 90-98, wherein the cancer is selected from pancreatic cancer, ovarian cancer, prostate cancer and glioblastoma.

### Embodiment 100

The method of any one of embodiments 90-99, wherein the individual comprises an aberration in the tumor suppressor gene encoding.

### Embodiment 101

The method of any one of embodiments 91-100, wherein the individual comprises an aberration in the oncogene.

### Embodiment 102

The method of embodiment 100 or 101, wherein the individual is selected for treatment based on having the aberration in the gene encoding the tumor suppressor protein and/or the oncogene.

### Embodiment 103

The method of embodiment 101, wherein the siRNA targets a mutant form of the oncogene, and wherein the mutant form comprise the aberration in the oncogene.

### Embodiment 104

A method of treating a disease or condition in an individual comprising administering an effective amount of an mRNA encoding a therapeutic protein or a recombinant form thereof, wherein the therapeutic protein is selected from the group consisting of alpha 1 antitrypsin, frataxin, insulin, growth hormone (somatotropin), growth factors, hormones, dystrophin, insulin-like growth factor 1 (IGF1), factor VIII, factor IX, antithrombin III, protein C, β-Gluco-cerebrosidase, Alglucosidase-α, α-1-iduronidase, Iduronate-2-sulphatase, Galsulphase, human α-galactosidase A, α-1-Proteinase inhibitor, lactase, pancreatic enzymes (including lipase, amylase, and protease), Adenosine deaminase, and albumin.

### Embodiment 105

The method of embodiment 104, wherein the disease or condition is a protein deficiency disease or condition characterized by a deficiency in the therapeutic protein.

### Embodiment 106

The method of embodiment 104 or 105, wherein the therapeutic protein is factor VIII.

### Embodiment 107

The method of any one of embodiments 90-106, wherein the mRNA is administered intravenously or subcutaneously.

### EXAMPLES

Those skilled in the art will recognize that several embodiments are possible within the scope and spirit of this invention. The invention will now be described in greater detail by reference to the following non-limiting examples. The following examples further illustrate the invention but, of course, should not be construed as in any way limiting its scope.

Materials and Methods

Cell Penetrating Peptides:

PEP-1:
(SEQ ID NO: 71)
KETWWETWWTEWSQPKKKRKV
PEP-2:
(SEQ ID NO: 72)
KETWFETWFTEWSQPKKKRKV
VEPEP-3a:
(SEQ ID NO: 75)
βAKWFERWFREWPRKRR
VEPEP-3b:
(SEQ ID NO: 76)
βAKWWERWWREWPRKRR
VEPEP-6:
(SEQ ID NO: 77)
βALWRALWRLWRSLWRLLWKA
VEPEP-9:
(SEQ ID NO: 78)
βALRWWLRWASRWFSRWAWWR
ADGN-100a:
(SEQ ID NO: 79)
βAKWRSAGWRWRLWRVRSWSR
ADGN-100b:
(SEQ ID NO: 80)
βAKWRSALYRWRLWRVRSWSR

Stock solutions of peptides were prepared at 2 mg/mL in distilled water or 5% DMSO and sonicated for 10 min in a water bath sonicator then diluted just before use.

Cell Lines

Several cell lines were used, including stable EGFP expressing cell lines (GFP-U2OS, EGFP-JURKAT T, EGFP-HEK) as well as U2OS (ATCC® HTB-96™), primary human fibroblasts, Hep G2 (ATCC® HB-8065™), Human Embryonic Kidney (HEK293) (ATCC® CRL-1573™), Human Myelogenous Leukemia K562 cells (ATCC® CCL243™), Jurkat T cells (ATCC® TIB-152™), human ESCs (H9), and mouse ESCs (ESF 158). Cells were obtained from the American Type Culture Collection [ATCC].

### Example 1: ADGN-Peptides/mRNA Nanoparticle and Complexes Preparation, Size Distribution, In Vitro and In Vivo Use

Materials

Lipofectamine 3000, TranscriptAid T7 transcription and MEGAclear transcription Clean Up kits, GeneArt Genomic Cleavage Detection kit mix were obtained from Thermo Fisher life Science (France). HiScribe™ T7 ARCA mRNA Kit was obtained from New England Biolab. TranscriptAid T7 transcription kit. MEGAclear transcription Clean Up kit, GeneArt Genomic Cleavage Detection kit, and Platinum Green Hot Start PCR mix were obtained from Thermo Fisher life Science (France). All oligonucleotides were obtained from Eurogentec (Belgium).

Luciferase mRNA was obtained using HiScribe™ T7 ARCA mRNA Kit (New England Biolab). MRNA was synthesized using linear vector as DNA template (Luc2 pGL4-10) (Addgene) and purified by phenol:chloroform extraction. Synthesized mRNA was purified by LiCl precipitation, phenol:chloroform extraction followed by ethanol precipitation, then quantified by UV Light Absorbance. RNA concentration was determined by measuring the ultraviolet light absorbance at 260 nm. 18 μg of capped mRNA was obtained using 1 μg of DNA template and stored at −20° C. For in vivo studies CAS9 mRNA was obtained from ThermoFisher as a polyadenylated and Capped form.

The following peptide sequences were used. All peptides were obtained from GENEPEP Montpellier (FRANCE) as acetate salt form in order to facilitate solubility and in vivo application.

ADGN-106:
(SEQ ID NO: 77)
βALWRALWRLWRSLWRLLWKA
ADGN-100:
(SEQ ID NO: 79)
βAKWRSAGWRWRLWRVRSWSR

HepG2 cells were from ATCC (ATCC® CCL243™).

Methods

Peptide Stock Preparation

ADGN-100 and ADGN-106 peptides were stored in powder in plastic tube. Peptide powders are stable at room temperature, but should be stored at −20° C. or −80° C. Peptide powders were equilibrated at room temperature for 30 min before opening the tube to prevent peptide hydration. Peptide powders were first solubilized directly in the tube by adding pure DMSO (cell culture grade, SIGMA ref D2650-5X5ML) to obtain a concentration of 20 μl/mg of peptide. Then the DMSO solutions were diluted with appropriate volume of GIBCO sterile water (Cell culture Grade), according to the peptide concentration required. Peptide concentration of 550 μM was currently used. The water volume was added dropwise to the DMSO solution. The peptide solution was mix gently with vortex 2 min low speed and peptide Stock Solutions are sonicated 10 min in a water bath (Digital Ultrasonic Cleaning Bath BRANSON).

Peptide Stock Solutions can be stored at −80° C. for 1 month or at refrigerated conditions (2-8° C.) for 1 week. Peptide Stock Solution were aliquoted in 100 μl samples and stored at −80° C. Only one freeze-thaw is recommended. As standard control, peptide concentrations were validated based on UV absorbance at 280 nm using epsilon values according to the peptide used ADGN-100 [ε280:27500] and ADGN106: [ε280:28400]

Preparation of Final Peptide Solution for Complexation

Peptide stock solutions were diluted 3.7 folds in sterile water to obtain the final peptide solution to reach a concentration of 150 μM and sonicated 10 min in a water bath (Digital Ultrasonic Cleaning Bath BRANSON). We suggested diluting 100 μl of peptide stock solution with 270 μl of GIBCO sterile water to obtain a Final Peptide Solution of 370 μl volume. Final Peptide Solutions were stored at 4° C. and used within 10 hours of preparation. Volume can be adjusted depending on the number of transfection.

Complex Formation with mRNA

The following protocols are reported for the transfection of 2-5 10⁶ Cells or Cells at confluency of about 70-80%. cultured in a 35 mm dish corresponding to 1 well (6 well plate). Protocol can be adjusted for different number of cells, larger volume preparation and different plate formats.

ADGN peptide/mRNA particles were prepared at a 20:1 molar ratio of ADGN-Peptide/mRNA. 3 amounts of mRNA (0.1 μg, 0.5 μg and 2.0 μg) are described in the protocol.

First, mRNA (0.1, 0.5 or 2.0 μg) was diluted in 20 μl of sterile water (GBCO) at room temperature. 2 μl Final Peptide Solution was added for 0.1 μg of mRNA, or 10 μl Final Peptide Solution was added for 0.5 μg of mRNA or 40 μl Final Peptide Solution was added for 2 μg of mRNA to obtain a total volume of 22 μl, 30 μl or 60 μl, respectively. The volume was adjusted to 100 μl with sterile water. The peptide/mRNA solution was mixed gently with vortex for 1 minute at low speed and incubated for 30 min at room temperature for complex formation.

Just before transfection, the volume of the complex was adjusted to 200 μl by adding either sterile water containing 5% sucrose or 5% glucose. It was discovered that PBS and high salt concentration resulted in particle aggregation. Therefore, it is recommended to avoid them. It was also found that 50% DMEM or OPTIMEM could be used, depending on the sensitivity of the cell lines or cell types. After the volume adjustment, the complex solution was mixed gently with vortex for 1 minute at low speed, incubated for 5 min at 37° C., and used for cell transfection or in vivo administration.

Transfection Protocols

(i) Protocol for Adherent Cell Lines:

The following protocol is for 24 well plates format. Volume can be optimized for larger volume and different plate formats. Cells were cultured in Dulbecco's Modified Eagle's Medium (DMEM), supplemented with 2 mM glutamine, 1% antibiotics (streptomycin 10,000 μg/mL, penicillin, 10,000 IU/mL) and 10% (w/v) foetal calf serum (FCS), at 37° C. in a humidified atmosphere containing 5% CO₂. 24 well plates seeded with 150,000 cells the day prior to transfection were grown to 50-60% confluence and set up to be at about 70% confluences at the time of transfection.

Before transfection, cells were washed twice with DMEM. Cells were then overlaid with 0.2 ml of complex solution, mixed gently, and incubated for 10 minutes at 37° C. 0.4 ml of fresh OPTiMEM or DMEM was added and cells were incubated for 20 min at 37° C. 2 ml of Complete OPTiMEM (high glucose) or DMEM containing 15% FCS was then added in order to reach a final FCS concentration of 10%, without removing the overlay of ADGN-peptide/Cargo complexes. Cells were returned to the incubator (37° C., 5% CO₂) and assayed at 72 hours post transfection.

(ii) Protocol for Cell Lines in Suspension and T Cell:

Cells were cultured in high glucose DMEM media with 10% serum to be at about 70% confluence at the time of transfection. Before transfection, cells were harvested by centrifugation, then washed twice with DMEM. Cells are then resuspended in 0.2 ml of complex solution, mixed gently, and incubated for 10 min at 37° C. 0.4 mL of fresh OPTiMEM were then added and cells were incubated for 30 min at 37° C. 2 mL of complete OPTiMEM (high glucose) containing 12% FCS was then added in order to reach a final FCS concentration of 10%, without removing the overlay of ADGN-peptide/Cargo complexes. Cells were returned to the incubator (37° C. 5% CO₂) and assayed at 72 hours post transfection.

### Example 1a: Effect of Diluents on Nanoparticle Size of Peptide/mRNA Complexes

It was unexpectedly discovered that the transfection (in vitro and in vivo) with particles prepared in PBS was lower than expected. Since the peptide/mRNA complexes are neutral (zeta potential+10 mv to −10 Mv) it was not expected that salt or buffers would significantly affect the particles.

The ability of ADGN 100 and ADGN-106 peptides to form stable nanoparticles with mRNA was therefore analyzed in different buffer conditions. The following buffer conditions were evaluated, including sterile water, 5% Glucose, 5% Sucrose, 20% PBS (20% and 50%), Hepes pH 7.4 (50 mM), NaCl (40 mM, 80 mM, 160 mM), DMEM or OPTIMEM (20% and 50%).

Luciferase mRNA was obtained using HiScribe™ T7 ARCA mRNA Kit (New England Biolab), synthesized using plasmid DNA template (Luc2 pGL4-10)(Addgene) and purified by phenol:chloroform extraction. ADGN peptide/mRNA particles are prepared at a 20:1 molar ratio of ADGN-Peptide/mRNA.

Luc mRNA was mixed with ADGN-100 or ADGN-106 at 20:1 molar ratio in different buffer conditions. Luc mRNA (0.5 or 1.0 μg) in sterile water (GIBCO) were mixed with ADGN peptide (sterile water), volume for each sample was adjusted to 100 μl, with sterile water. Complexes were mixed gently with vortex for 1 minute at low speed and incubated 30 min at room temperature. Just before measurement, volume was adjusted to 200 μl by adding different buffers and the complexes were mixed gently with vortex for 1 minute at low speed and incubated 5 min at 37° C. The particle sizes and zeta potential were measured on DLS NanoZS (Malvern Ltd). The mean size and the polydispersity of the ADGN/mRNA complexes were determined at 25° C. for 3 minute per measurement and zeta potential was measured with Zetasizer 4 apparatus (Malvern Ltd).

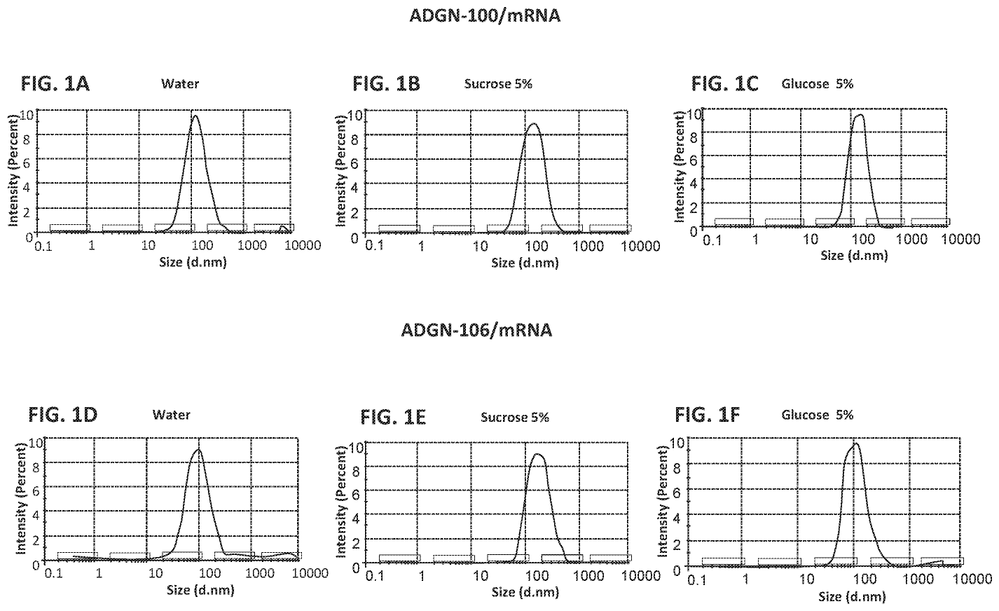

Data are shown in FIGS. 1-4 and Table 1 for a mean of 3 separate experiments.

TABLE 1

| | ADGN-100 | | | ADGN-106 | | |
|---|---|---|---|---|---|---|
| BUFFERS | Size (nm) | PI | zeta (mV) | Size (nm) | PI | zeta (mV) |
| WATER | 115.2 ± 5 | 0.244 ± 0.1 | −6.1 ± 0.2 | 98 ± 4 | 0.267 ± 0.1 | 7.0 ± 0.5 |
| SUCROSE (5%) | 122.5 ± 5 | 0.264 ± 0.1 | −7.0 ± 0.5 | 115.4 ± 5 | 0.248 ± 0.1 | 7.3 ± 0.7 |
| GLUCOSE (5%) | 127.5 ± 5 | 0.258 ± 0.1 | −6.7 ± 0.8 | 101.2 ± 5 | 0.252 ± 0.1 | 5.3 ± 0.7 |
| HEPES (7.4) | 107.1 ± 7 | 0.248 ± 0.1 | −7.2 ± 1 | 101.4 ± 6 | 0.258 ± 0.1 | 8.8 ± 0.3 |
| DMEM (20%) | 131.2 ± 2 | 0.256 ± 0.1 | −12 ± 2 | 125.4 ± 9 | 0.276 ± 0.1 | 9.1 ± 1 |
| DMEM (50%) | 243.2 ± 8 | 0.262 ± 0.1 | −12 ± 0.5 | 269.7 ± 8 | 0.312 ± 0.2 | 11 ± 2 |
| NACL(40 mM) | 170.1 ± 8 | 0.261 ± 0.1 | −5.4 ± 0.5 | 102.4 ± 8 | 0.241 ± 0.2 | 9.6 ± 1 |
| NACL (80 mM) | 195.1 ± 8 | 0.254 ± 0.1 | −5.2 ± 0.5 | 185.5 ± 9 | 0.261 ± 0.2 | 11.5 ± 3 |
| NACL (160 mM) | 691.2 ± 8 | 0.265 ± 0.1 | — | 542.4 ± 8 | 0.271 ± 0.2 | — |
| PBS (20%) | 1024 ± 60 | 0.281 ± 0.1 | — | 882 ± 60 | 0.278 ± 0.2 | — |
| PBS (50%) | 1756 ± 80 | 0.286 ± 0.1 | — | 1126 ± 80 | 0.259 ± 0.2 | — |

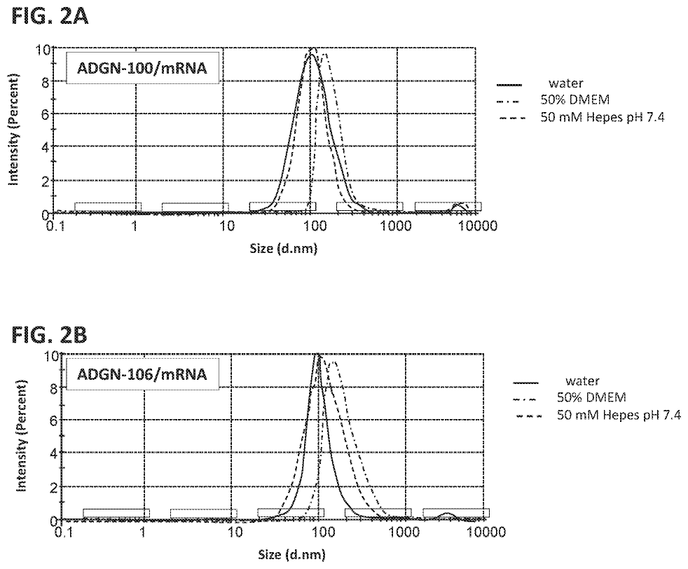

As shown in FIGS. 1A-1F and 2A-2B, both ADGN-100 and ADGN-106 peptides were able to form stable nanoparticles with mRNA in water, glucose (5%), sucrose (5%) and 50 mM Hepes pH 7.4 with a mean diameters ranging between 98 and 130 nm and polydispersity index (PI) of 0.25-0.27. No major differences were observed between the 2 peptides. Particle charge obtained by zeta potential, with mean values ranging from −6.2 mV to −7.2 mV, for ADGN-100 and from +5.3 mV to +8.8 mV, for ADGN-106, respectively.

The particle size of ADGN-100/mRNA and ADGN-106/mRNA complexes was evaluated in standard cell culture medium such as DMEM or OPTIMEM (20% and 50%). As shown in FIGS. 2A-2B, the presence of DMEM slightly increases the size of the particles. The particle charge obtained by zeta potential, in medium condition are similar to those obtained in sucrose or water with mean values of −12 mV for ADGN-100 and of +11 mV, for ADGN-106, respectively.

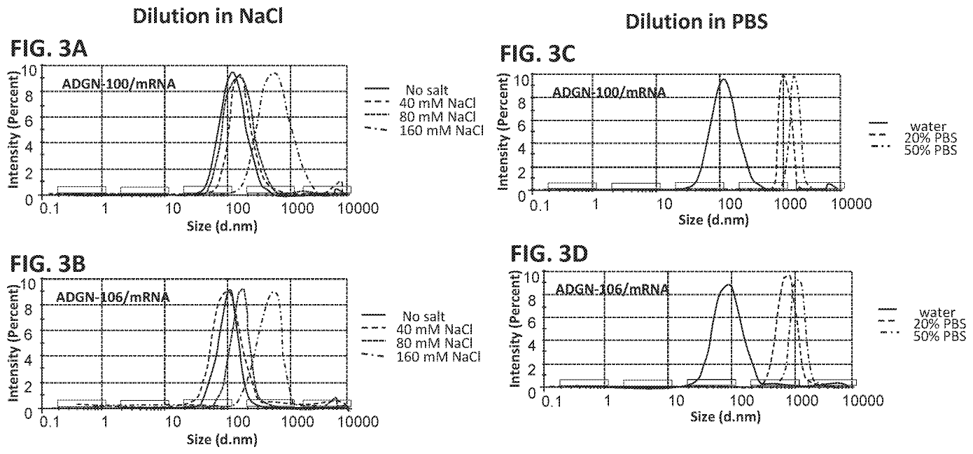

In contrast, for both ADGN peptides, the presence of salt at high concentrations, either phosphate in PBS or NaCl, resulted in a larger particle size. As shown in FIGS. 3A-3B, increasing NaCl concentrations from 40 mM to 160 mM, increases particle size by 5 to 7 folds, with mean diameters up to 691 nm and 542 nm for ADGN-100/mRNA and ADGN-106/mRNA complexes, respectively. The presence of 50% PBS increases the particle size by 11 and 16 folds, for ADGN-106 and ADGN-100 respectively. The results unexpectedly demonstrated that water, sucrose 5% and glucose 5% resulted in the smallest particles for ADGN-100/mRNA and ADGN-106/mRNA complexes. In contrast, for both ADGN-100 and ADGN-106, the presence of salt or phosphate unexpectedly resulted in the formation of much larger particles that may explain the observed reduction transfection potency and potentially could also increase the risk of toxicity of the particles.

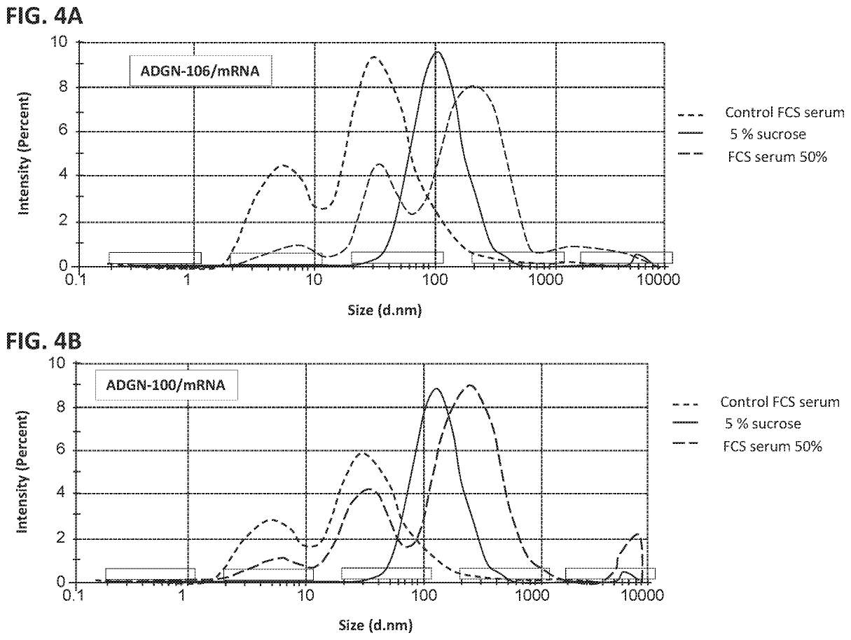

Serum (FCS) is an essential component of cell culture medium that has been reported to reduce efficiency of numerous delivery agents. In the case of numerous sensitive cell types, serum is present during transfection or added just after transfection, to limit cell death. Therefore the impact of the presence of serum on the particle size and charges was investigated. The mean size and charge of ADGN-100/mRNA and ADGN-106/mRNA particles were evaluated in the presence of 20% and 50% FCS. The ADGN-peptide/mRNA particles in water, sucrose 5% or glucose 5%, were then incubated for 30 min in different serum conditions (20% and 50%). The mean size and the polydispersity of the ADGN/mRNA complexes were determined at 25° C. for 3 min per measurement with Zetasizer 4 apparatus (Malvern Ltd). Data are shown in FIGS. 4A-4B and Table 2 for a mean of 3 separate experiments.

TABLE 2

| | ADGN-100 | | ADGN-106 | |
|---|---|---|---|---|
| BUFFERS | Size (nm) | PI | Size (nm) | PI |
| WATER | 115.2 ± 5 | 0244 ± 0.1 | 98 ± 4 | 0.267 ± 0.1 |
| + FCS 50% | 464.5 ± 5 | 0293 ± 0.1 | 428.1 ± 4 | 0.291 ± 0.1 |
| SUCROSE (5%) | 122.5 ± 5 | 0.264 ± 0.1 | 115.4 ± 5 | 0.248 ± 0.1 |
| + FCS 20% | 183.1 ± 5 | 0.273 ± 0.1 | 163.2 ± 5 | 0.267 ± 0.1 |
| + FCS 50% | 205.3 ± 5 | 0.298 ± 0.1 | 183.1 ± 5 | 0.273 ± 0.1 |
| GLUCOSE (5%) | 127.5 ± 5 | 0.258 ± 0.1 | 101.2 ± 5 | 0.252 ± 0.1 |
| + FCS 20% | 174.2 ± 5 | 0.268 ± 0.1 | 161.3 ± 5 | 0.268 ± 0.1 |
| + FCS 50% | 192.2 ± 5 | 0.273 ± 0.1 | 173.2 ± 5 | 0.278 ± 0.1 |

As reported in FIGS. 4A-4B, free serum is characterized by two distinct peaks at 5+1 nm and 45±10 nm. When ADGN:mRNA nanoparticles in sucrose (5%) or glucose (5%) solution were mixed with serum (50% or 20% final concentration), a third peak was obtained with a size around 200 nm. When ADGN:mRNA nanoparticles in water were mixed with serum (50% or 20% final concentration), a third peak was obtained with a size around 450 nm. These results suggest that size of the particle is increased by interaction with serum protein or other serum components and nanoparticles are surrounded by a “protein corona” due to dynamic adsorption of the serum proteins at the surface of the particles. The association of serum proteins around the nanoparticle is limited in sucrose or glucose solutions. The association is further confirmed by modification of surface charge measurements, showing a switch between a positive zeta potential of +7.3±2 mV to a negative one, −10±3 mV after serum addition for ADGN-106/mRNA particles and an increase from −7.0±0.5 mV to −15.2±0.5 mV for ADGN-100/mRNA complexes in sucrose solution.

The results demonstrated that ADGN-100 and ADGN-106 form stable nanoparticles with mRNA, when stored in water, in glucose or sucrose solutions. Nanoparticles stored in glucose or sucrose are stable in serum conditions with limited association of serum proteins around the nanoparticle. In contrast high salt concentrations or PBS induced particle size increase.

### Example 1b: ADGN-Peptides Promote mRNA Delivery in HepG2 Cells

ADGN-100/mRNA and ADGN-106/mRNA were evaluated for cellular delivery of Luciferase mRNA in HEPG2 cells.

Luc mRNA (0.5 or 1.0 μg) in sterile water (GIBCO) were mixed with ADGN peptide (sterile water). The volume for each sample was adjusted to 100 μl, with sterile water and mix gently with vortex for 1 minute at low speed. Samples were incubated 30 min at room temperature. Just before transfection, the volume was adjusted to 200 μl by adding different buffers. The following buffer conditions were evaluated: including sterile water, 5% Glucose, 5% Sucrose, 20% PBS, 50% PBS, Hepes pH 7.4 (50 mM), NaCl (40 mM, 80 mM, 160 mM), DMEM (50%). Just prior to transfection, samples were mixed gently with vortex 1 min low speed and incubated 5 min at 37° C.

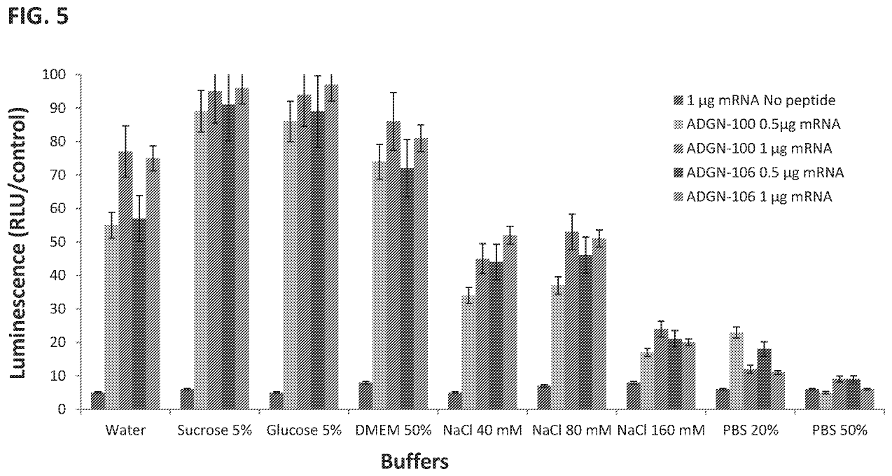

HepG2 cells were cultured in 24 well plates. I00000 HepG2 cells were seeded the day prior to transfection and grown to 50-60% confluence. Before transfection, cells were washed twice with DMEM and DMEM was removed gently. Cells were then overlaid with 0.2 ml of complex solution, mixed gently, and incubated for 10 min at 37° C. 0.4 mL of fresh DMEM without serum and antibiotics were added and cells were incubated for 2 hours at 37° C. 2 mL of complete DMEM containing 15% FCS were then added without removing the overlay of ADGN/mRNA complexes. Cells were returned to the incubator (37° C., 5% CO₂) and assayed at 30 hours post transfection for luciferase expression. Results are shown in FIG. 5 regarding percentage of RLU (luminescence) relative to untreated cells.

High level of luciferase expression was observed for both ADGN peptides in sucrose (5%) and glucose (5%). The luciferase expression efficiency was reduced by 20% in DMEM and by 30-40% when the particles are in water. Incubation of the particles in the presence of NaCl 40 mM or 80 mM reduced efficiency by 50%. Finally, the presence of 160 mM NaCl or PBS dramatically reduced transfection efficiency by 80-90%. The results demonstrated that ADGN-100 and ADGN-106 promote efficient delivery of mRNA in HepG2 cells. The results also showed that ADGN/mRNA particles in sucrose and glucose led to high transfection efficiency. In contrast, the presence of salt or phosphate, increased size of the particles and resulted in poor transfection possibly preventing entry to the cell via a non-endosomal pathway.

### Example 1c: ADGN-Peptides Promotes mRNA Delivery In Vivo

Stable ADGN-100/mRNA and ADGN-106/mRNA were evaluated for in vivo delivery of Luciferase mRNA via intravenous administration.

Luc mRNA (10 μg) in sterile water (GIBCO) were mixed with ADGN peptide (sterile water), volume for each sample was adjusted to 100 μl, with sterile water. Samples were mixed gently with vortex for 1 minute at low speed and incubated for 30 min at room temperature. Just before injection, volume was adjusted to 200 μl by adding different buffers (sucrose 5%, glucose 5%, NaCl 80 mM or PBS 20% final concentration) Samples were mixed gently with vortex for 1 minute at low speed and incubated for 5 min at 37° C., then immediately injected into mice. Mice from Group 1 and 2 received IV injection of 100 μl ADGN-100/mRNA complex or ADGN-106/mRNA solutions containing 10 sg of Luc mRNA respectively, (3 animals per group). As control, mice from group 3 (2 animals per group) received IV injection of 100 μl saline solution.

mRNA Luc expression was monitored by bioluminescence. Bioluminescence imaging was performed at Day 3 and 6. Mice received an i.p. injection of 150 μg/g luciferin for non-invasive bioluminescence imaging (IVIS Kinetic; PerkinElmer, Waltham. Mass., USA).

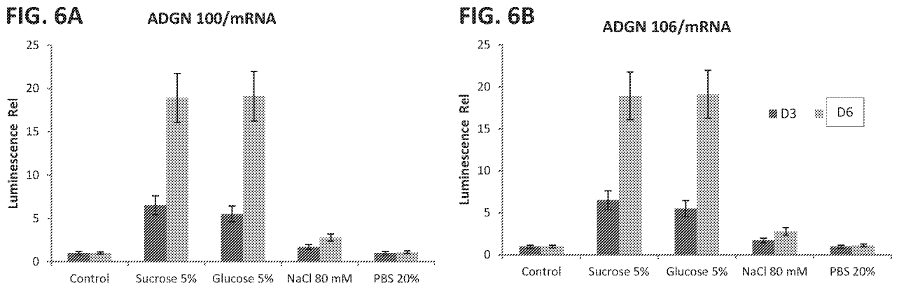

Semi-quantitative data of luciferase signal in the liver were obtained using the manufacturer's software (Living Image; PerkinElmer). Results were then expressed as values relative to day 0 and shown in FIGS. 6A and 6B.

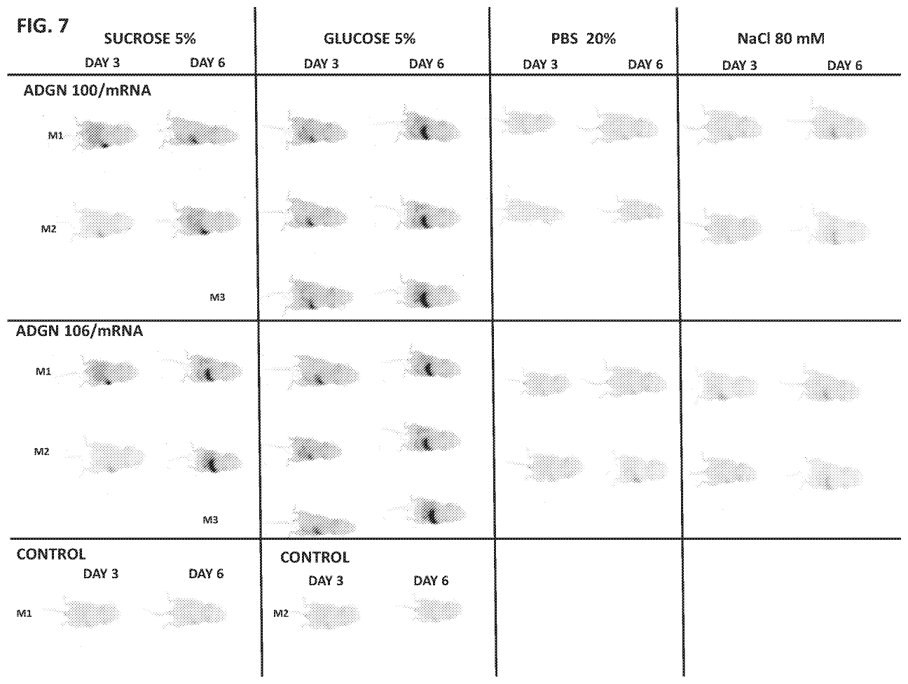

As shown in FIGS. 6A-6B and 7, both ADGN-00and ADGN-106 mediated in vivo mRNA delivery and mRNA expression was observed in the liver starting at day 3 with optimal values at day 6. For both peptides, higher Luciferase expression in the liver was obtained with particles in glucose and sucrose. In contrast, negligible luciferase expression was obtained with particles in PBS and only 5-10% expression with particles in NaCl 80 mM as shown in FIGS. 6A and 6B.

The results confirm that glucose and sucrose are the best diluents for both in vitro and in vivo mRNA delivery. Large aggregates induced by salt are not able to enter the cells and to promote in vivo delivery.

### Example 2: Peptide Mediated PTEN Tumor Suppressor mRNA Delivery in Tumor Cells

Phosphatase and tensin homologue deleted on chromosome 10 (PTEN) is as a well-known tumor suppressor that has both phosphatase-dependent and -independent roles. PTEN is one of the most frequently disrupted tumor suppressors in cancer. By suppressing the phosphoinositide 3-kinase (PI3K)-AKT-mammalian target of rapamycin (mTOR) pathway through its lipid phosphatase activity. PTEN governs a plethora of cellular processes including survival, proliferation, energy metabolism and cellular architecture. Consequently, mechanisms regulating PTEN expression and function, including transcriptional regulation, post-transcriptional regulation by non-coding RNAs, post-translational modifications and protein-protein interactions, are all altered in cancer. Lesions in the PTEN gene, located on chromosome 10q23, occur at a significant rate in the majority of human tumor subtypes, and this locus is thought to have the highest preference for loss in humans. Inactivation of PTEN is a key event in tumorigenesis and tumor development, and it has the highest frequency of mutation in cancer after the P53 gene. Currently, the tumor Suppressing mechanism of the PTEN gene likely involves several candidate pathways, including the FAK pathway, the MAPK pathway, and the PI3K′AKT pathway.

Given the high frequency of PTEN deficiency across cancer subtypes, therapeutic approaches that exploit PTEN loss-of-function could provide effective treatment strategies. In order to propose a new strategy to restore level of wild type PTEN in cancer cells, we have evaluated the potency of ADGN peptides (ADGN-100 and ADGN-106) to deliver PTEN mRNA in several tumor cells all exhibiting loss in PTEN function associated to either mutations in PTEN gene or loss of expression in PTEN.

Materials

PTEN mRNA was obtained using HiScribe™ T7 ARCA mRNA Kit (New England Biolab). MRNA was synthesized using linear vector PGL-PTEN as DNA template (Addgene 13039) and purified by phenol:chloroform extraction. Synthesized mRNA was purified by LiCl precipitation, phenol:chloroform extraction followed by ethanol precipitation, then quantified by UV Light Absorbance. RNA concentration was determined by measuring the ultraviolet light absorbance at 260 nm. 18 μg of capped mRNA was obtained using 1 μg of DNA template and stored at −20° C. For in vivo studies CAS9 mRNA was obtained from ThermoFisher as a polyadenylated and Capped form.

The following ADGN peptide sequences were used:

ADGN-106: βALWRALWRLWRSLWRLLWKA (SEQ ID NO: 77)

ADGN-100: βAKWRSAGWRWRLWRVRSWSR (SEQ ID NO: 79)

Pancreas cancer cells PANC-1, human Glioma cell U25, Prostate cancer cells PC3, ovarian cancer cells SKOV3 and human fibroblast HS69 were obtained from ATCC.

Methods

The following protocols were used for the transfection of 2-5 10⁶ Cells or Cells at confluency of about 70-80%. cultured in 24 well plates. ADGN peptide/mRNA particles were prepared at a 20:1 molar ratio of ADGN-Peptide/mRNA; using two doses of mRNA (0.5 μg and 1.0 μg). PTEN mRNA (0.5 or 1.0 μg) were diluted in 20 μl of sterile water (GBCO) at room temperature. 10 μl Final Peptide Solution was added for 0.5 μg of mRNA, or 20 μl Final Peptide Solution was added for 1 μg of mRNA to obtain a total volume of 30 μl or 40 μl, respectively. The volume of the peptide/mRNA solution was adjusted to 100 μl with sterile water. The peptide/mRNA solution was mixed gently with vortex for 1 minute at low speed and incubated for 30 min at room temperature. Just before transfection, the volume was made up to 200 μl by adding sterile water containing 5% sucrose. The solution was then mixed gently with vortex for 1 minute at low speed and incubated for 5 min at 37° C. Then the solution was used for cell transfection or in vivo administration.

The following protocol is reported for 24 well plates format and volume can be optimized for larger volume and different plate formats. Cells were cultured in Dulbecco's Modified Eagle's Medium (DMEM), supplemented with 2 mM glutamine, 1% antibiotics (streptomycin 10,000 μg/mL, penicillin, 10,000 IU/mL) and 10% (w/v) foetal calf serum (FCS), at 37° C. in a humidified atmosphere containing 5% CO₂. 24 well plates seeded with 150,000 cells the day prior to transfection were grown to 50-60% confluence and set up to be at about 70% confluences at the time of transfection.

Before transfection, cells are washed twice with DMEM. Cells were then overlaid with 0.2 ml of complex solution, mixed gently, and incubated for 10 min at 37° C. 0.4 mL of fresh DMEM were added and cells were incubated for 20 min at 37° C. 2 mL of complete OPTiMEM (high glucose) or DMEM containing 15% FCS were then added in order to reach a final FCS concentration of 10%, without removing the overlay of ADGN-peptide/Cargo complexes.

Cells were returned to the incubator (37° C., 5% CO₂) and assayed at 72 hours post transfection. The level of PTEN expression was analyzed by western blot using PTEN monoclonal antibody (A17 Thermofisher). The impact of PTEN expression was monitored by proliferation assays using an MT assay and apoptosis/cell cycle progression using a flow cytometry assay. Cell apoptosis rate (expressed as a percentage) and cell cycle stage were measured using a Propidium Iodide (PI) staining kit (Sigma-Aldrich) and APO BrDu kit (Thermofisher).

Results

For all cell types, the level of PTEN was evaluated by western blots using PTEN antibody (Thermofisher). Image Lab 4.1 software was used to analyze the protein bands and relative protein expression level was normalized with reference to β-actin (Abcam Inc.).

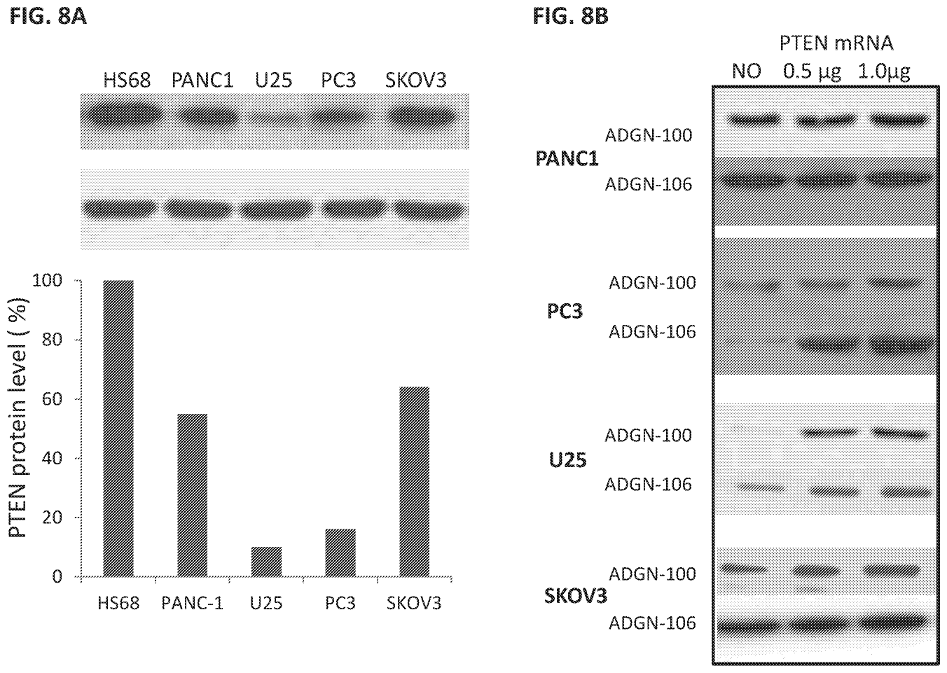

As shown in FIG. 8A, level of PTEN expression was dependent on the cell types. A very low PTEN expression was observed in Glioma U25 (10%) and PC3 cells (16%). In PANC-1 and SKOV3 ovarian carcinoma cells, the level of PTEN corresponded to 55% and 63% of that of non-transformed cells (HS-68). Differences in PTEN expression levels were in agreement with previously reported studies; showing that loss in PTEN activity is mainly associated to mutations in PTEN gene (Min Sup Song et al. 2013, Nature Rev Mol Cell Biol. Dillon & Tyler. Curr Drug Targets. 2014 15(1): 65-79.)

ADGN-100/mRNA and ADGN-106/mRNA complexes were evaluated for cellular delivery of PTEN mRNA in the different cancer cell types. Cells were transfected with 0.5 and 1 μg mRNA in complex with either ADGN-100 and ADGN-106 and level of PTEN expression was analyzed by western blot after 48 hrs. As shown in FIG. 8B, in all the cases, ADGN-100 and ADGN-106 promote efficient delivery of PTEN mRNA leading to PTEN protein expression. In U25 and PC3, level of PTEN protein is fully recovered in comparison to control cell types.

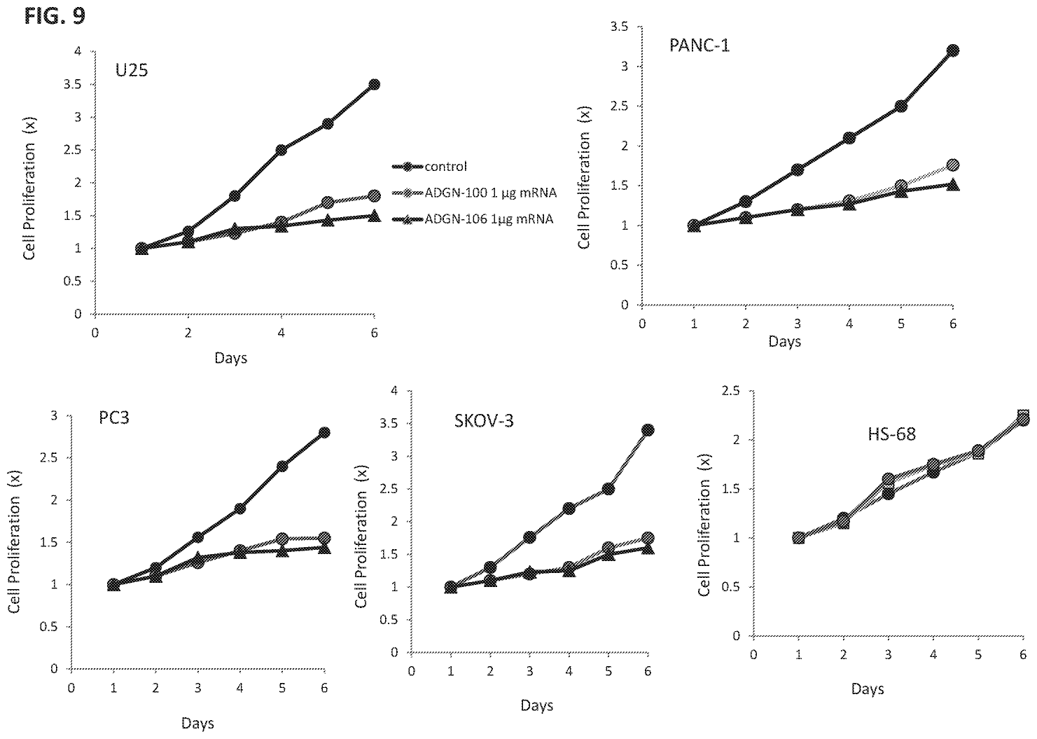

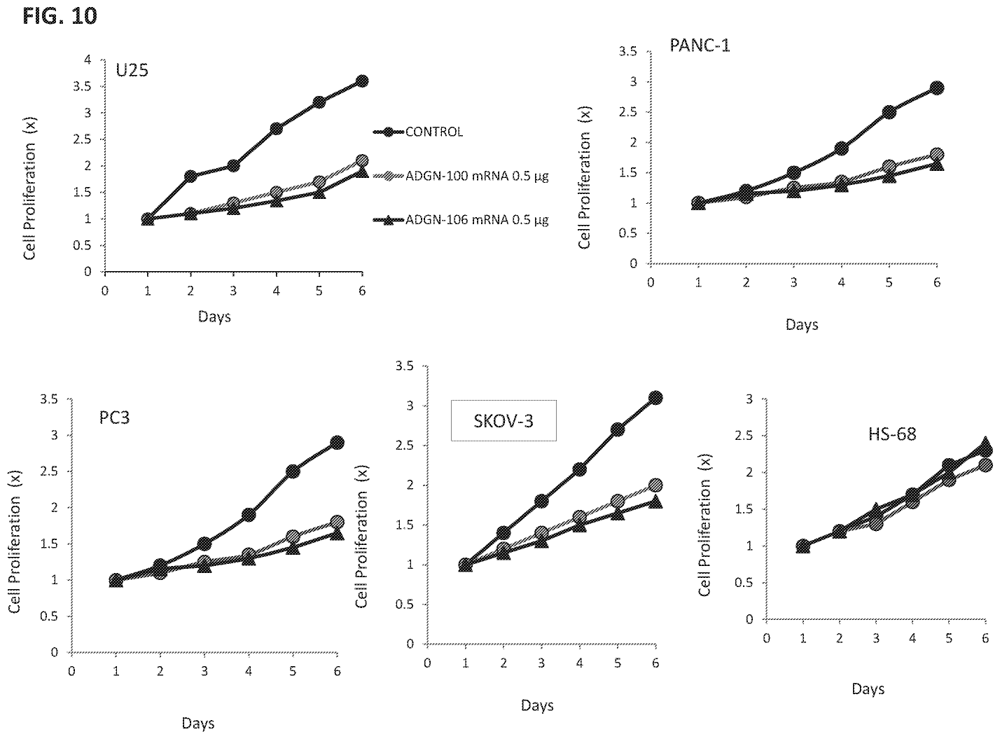

The impact of PTEN expression on cancer cell regulation was then evaluated by monitoring cell proliferation over a period of 6 days. As shown in FIGS. 9 and 10, in all cell types, expression of PTEN mRNA directly correlated to an inhibition of cell proliferation. The reduction in growth curve of cancer cells is marked at Day 6. The results indicate that wild type PTEN expression strongly decrease the viability of the cells or slow down their proliferation.

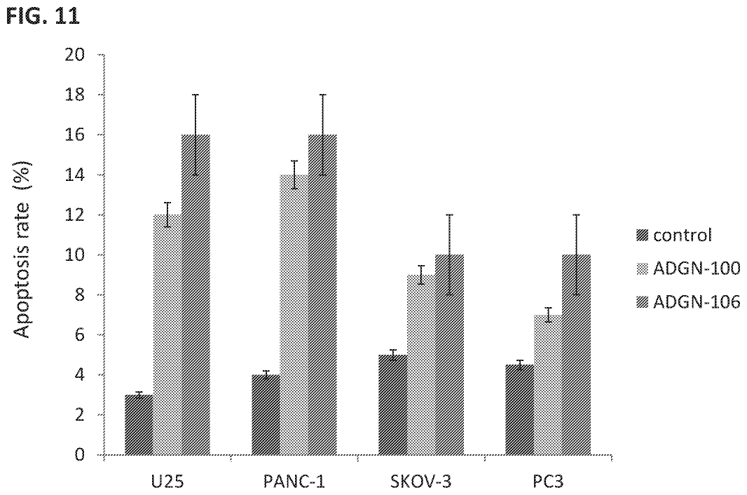

The flow cytometry data (FIG. 11) demonstrated that the rate of cell apoptosis was significantly enhanced in cells expressing wild-type PTEN, compared with the control cells. Apoptosis level was increased by 5 folds in U25 and PANC-1 cells and by 2.5 folds in SKOV-3 and PC3 cells.

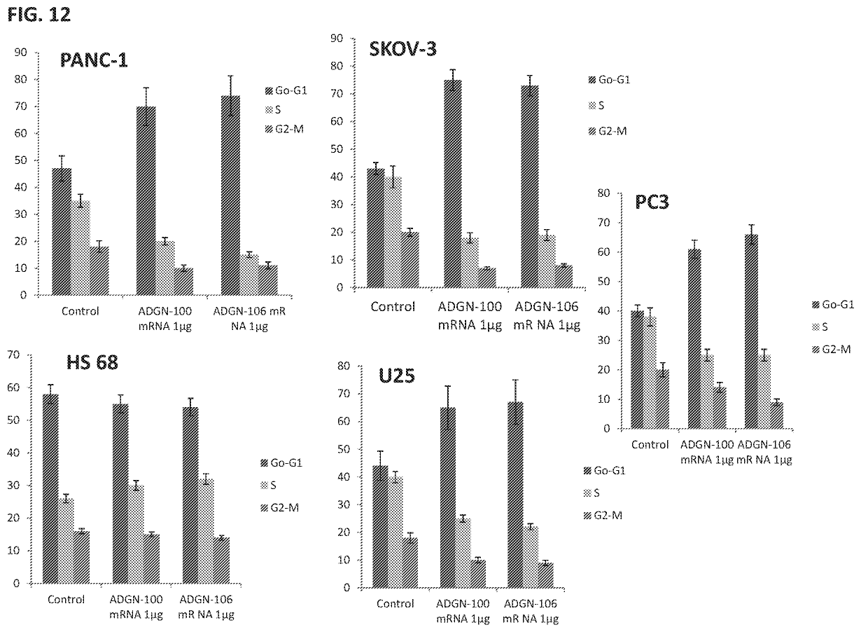

Cell cycle stage were measured by cytometry using a PI (Propidium Iodide) staining kit Similarly, compared with the control cells, there was an increased proportion of GO-G1 phase cells in the wild-type PTEN transfected cells. The number of cells in GO-G1 increased from 43-47% to 72-74%. (FIG. 12). In contrast, no changes in cell cycle progression were observed for HS68 in the presence of wild type PTEN.

The results demonstrate that ADGN-100 and ADGN-106 are potent agents for the delivery of PTEN mRNA in cancer cells. ADGN peptides mediated mRNA delivery leads to a large expression of exogenous wild type PTEN and rescues PTEN functions by profoundly inhibiting the growth of tumor cells, promoting cellular apoptosis, and causing cell cycle arrest at the G1 phase.

### Example 3: Peptide-Mediated PTEN Tumor Suppressor mRNA Delivery In Vivo Pancreas Tumor Xenograft Model

We have evaluated the potency of ADGN peptides (ADGN-100 and ADGN-106) to deliver PTEN mRNA in vivo in a pancreas tumor mouse model. Female nude mice 6-weeks of age were implanted in the pancreas with Human pancreatic carcinoma cell lines (Panc1-Luc) (20×10⁶ cells in 200 μl PBS). The animals were kept under pathogen-free conditions and fed and watered ad libitum, in cages of 2 to 4 animals (in compliance with recommended area surface/animal), in a dedicated room with a 12 h/12 h light/dark cycle at a constant temperature of 22° C. A period of 3 weeks was allowed for tumor development before the beginning of the experiments. Six groups of mice were used including 2 control groups (G1 & G2, 2 animals per group) and 4 treatment groups (G3-G6) (3 animals per group). The different groups are:

G1-: Control Untreated mice (CONTROL) (2 animal/group)

G2: Naked mRNA 10 ug (NAKED) (2 animal/group)

G3: ADGN-100/5 μg PTEN mRNA dose 0.25 mg/kg

G4: ADGN-100/10 μg PTEN mRNA dose 0.5 mg/kg

G5: ADGN-106/5 μg PTEN mRNA dose 0.25 mg/kg

G6: ADGN-106/10 μg PTEN mRNA dose 0.5 mg/kg

Animal (G2-G6) were injected with naked mRNA or ADGN/mRNA complex every 7 days. Mice received IV tail-vein injection of 100 μl ADGN/mRNA complex in saline buffer (90 mM NaCl). Tumor size was evaluated by bioluminescence imaging. Mice received an i.p. injection of 150 μg/g luciferin for non-invasive bioluminescence imaging (IVIS Kinetic; PerkinElmer, Waltham, Mass., USA). Semi-quantitative data of luciferase-positive tumor cell signals were obtained using the manufacturer's software (Living Image; PerkinElmer). Results were expressed as photons/second (photons/s). Bioluminescence imaging was performed at day 0, 7, 14, 20, 26 and 33. Results were then expressed as values relative to day 0. At Day 33 animals were sacrificed and tumors were harvested.

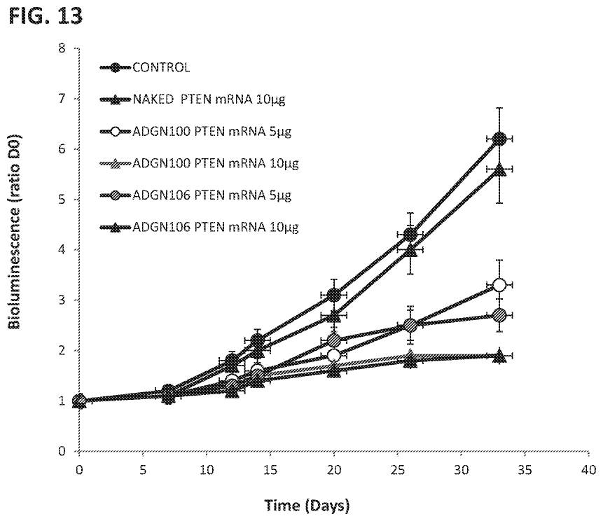

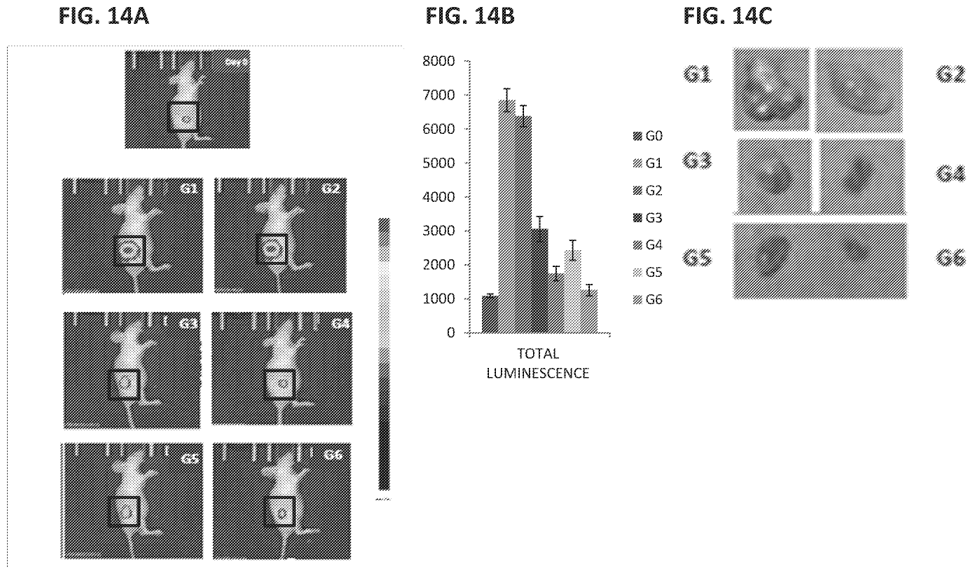

As shown in FIGS. 13 and 14A-14C, in the control group and naked mRNA group, tumor size increased by 5.5 to 6 folds over a period of 33 days. In contrast, IV administration of 5 μg of PTEN mRNA using ADGN-100 or ADGN-106 limited the tumor growth to 2.5 and 2.8 fold. Administration of 10 μg PTEN mRNA in complex with ADGN-100 or ADGN-106 significantly inhibits tumor growth, size increasing only by 1.5 fold. The results demonstrated that the ADGN peptides mediate efficient wild type PTEN mRNA delivery, restore PTEN function and inhibit pancreas tumor growth.

We next evaluated to what extent restoring PTEN function via mRNA delivery can inhibit metastases progression. Female nude mice 6-weeks of age were implanted in the pancreas orthotopically with Human pancreatic carcinoma cell lines (Panc1-Luc) (20×10⁶ cells in 200 μl PBS). A period of 6 weeks was allowed for tumor and metastases development before the beginning of the experiments. After 6 weeks, animal were injected with control saline or ADGN/PTEN complexes at day 0 and Day 3. Mice received IV injection of 100 μl ADGN-106/10 μg PTEN mRNA dose 0.5 mg/kg (Group G2) complex in saline buffer (90 mM NaCl) and control mice (G1) were injected with saline buffer solution. Tumor size was evaluated by bioluminescence imaging at day 0 and day 7.

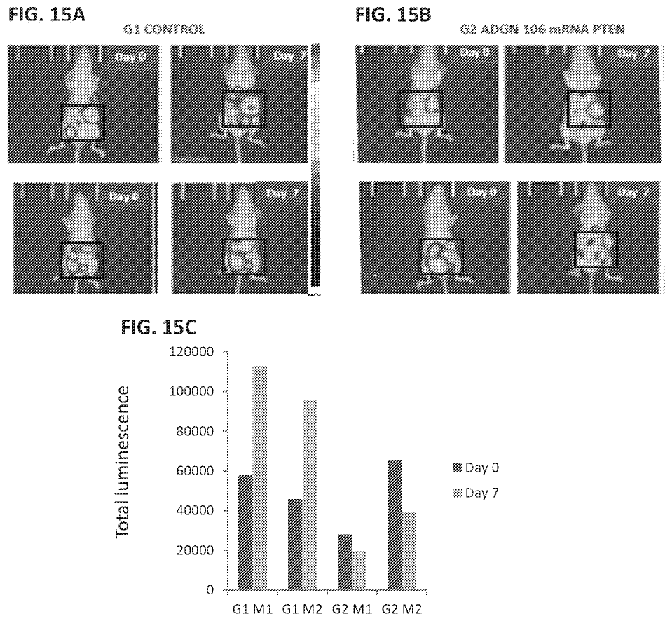

As shown in FIGS. 15A-15C, ADGN-106 mediated wild type PTEN mRNA delivery significantly limited metastasis development. Analyses demonstrated that total luminescence in the control group increased by 2 folds in 7 days, with several metastases development. In contrast to the control group, mice injected with ADGN/mRNA peptides decreased total luminescence by 12 to 40% in comparison to Day 0 and blocks metastasis establishment.

### Example 4: Peptide-Mediated Co-Delivery of PTEN (Tumor Suppressor) mRNA and KRAS (Oncogene) siRNA in Pancreas Tumor Animal Model

ADGN-106 has been evaluated for in vivo co-administration of PTEN mRNA and KRAS siRNA. The normal KRAS protein performs an essential function in normal tissue signaling, and the mutation of a KRAS gene is an essential step in the development of many cancers. Herein we combined siRNA targeting of the KRAS oncogene together with mRNA to restore PTEN tumor suppressor function and block the expression of the KRAS oncogene. This offers new perspective for anticancer targeting approach.

We have first validated the impact of delivery KRAS siRNA in cultured cancer cell lines. We have selected siRNA GUUGGAGCUUGUGGCGUAGTT-3′ (sense) (SEQ ID NO: 83) and 5′-CUACGCCACCAGCUCCAACTT-3′ (anti-sense) (SEQ ID NO: 84) to target specifically the KRAS G12C mutation. KRAS siRNA was first evaluated on cultured cancer cells. KRAS siRNA (10 nM and 40 nM) were associated to ADGN-106 at 20/1 peptide molar ratio. Pancreas cancer cells PANC-1 Human Glioma cell U25, Prostate cancer cells PC3, ovarian cancer cells SKOV3 and human fibroblast HS68 were transfected with ADGN-106: KRAS siRNA complexes, then both level of KRAS expression and proliferation were measured 48 hours and 5 days later.

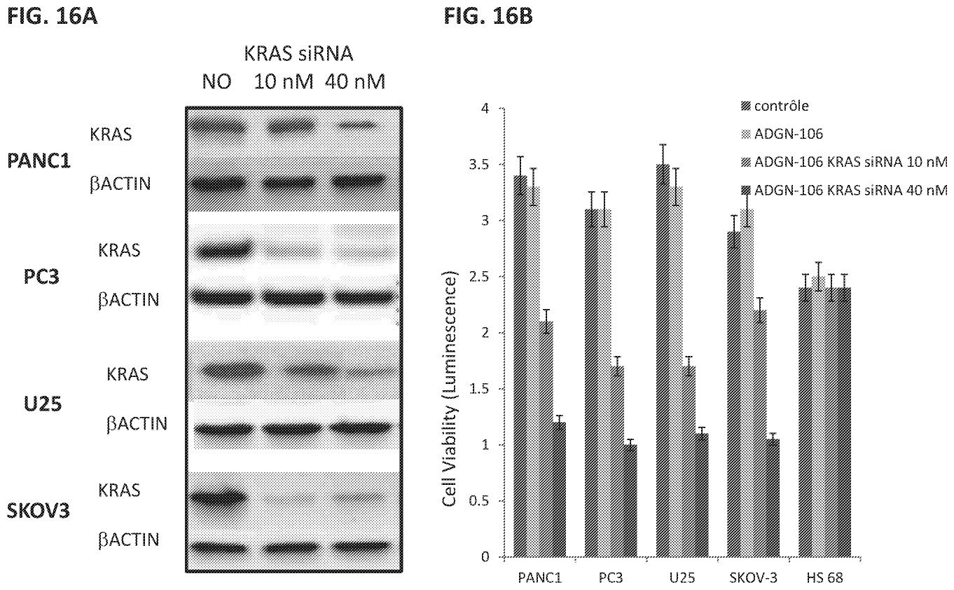

As shown in FIG. 16A, western blot analysis using mouse monoclonal anti-KRAS (Santa Cruz, Santa Cruz, Calif.), and for control, mouse monoclonal anti-Actin (Sigma, St. Louis, Mo.), revealed that the level of KRAS was reduced by more than 80% using 40 mM siRNA concentration whatever the cancer cell types. FIG. 16B shows cell viability measured 5 days post-transfection using CellTiter-Glo Luminescent Cell Viability Assay (Promega). The results demonstrated that ADGN-106 mediated KRAS siRNA induced a marked knockdown of KRAS mutants, which is directly correlated to a significant decrease in cancer cell proliferation. In contrast, no changes in proliferation were obtained on non-transformed HS68 fibroblast cells.

Female nude mice 6-weeks of age were injected with Human pancreatic carcinoma cell lines (Panc1-Luc) (20×10⁶ cells in 200 μl PBS). The animals were kept under pathogen-free conditions and fed and watered ad libitum, in cages of 2 to 4 animals (in compliance with recommended area surface/animal), in a dedicated room with a 12h/12 h light/dark cycle at a constant temperature of 22° C. A period of 3 weeks was allowed for tumor development before the beginning of the experiments. Animal were treated every 7 days. Mice received IV injection of 100 μl ADGN complexes in saline buffer (90 mM NaCl) as described in the following groups:

G1: Control Untreated mice (CONTROL)

G2: ADGN-106/10 μg PTEN mRNA dose 0.5 mg/kg

G3: ADGN-106/10 μg siRNA KRAS dose 0.5 mg/kg

G4: ADGN-106/10 μg siRNA KRAS dose 0.5 mg/kg; ADGN-106/5 μg PTEN mRNA dose 0.25 mg/kg

Tumor size was evaluated by bioluminescence imaging. Mice received an i.p. injection of 150 μg/g luciferin for non-invasive bioluminescence imaging (IVIS Kinetic; PerkinElmer, Waltham, Mass., USA). Semi-quantitative data of luciferase-positive tumor cell signals were obtained using the manufacturer's software (Living Image; PerkinElmer). Results were expressed as photons/second (photons/s). Bioluminescence imaging was performed at day 0, 7, 14, 20 and 26. Results were then expressed as values relative to day 0. At Day 26 animals were sacrificed and tumors were harvested.

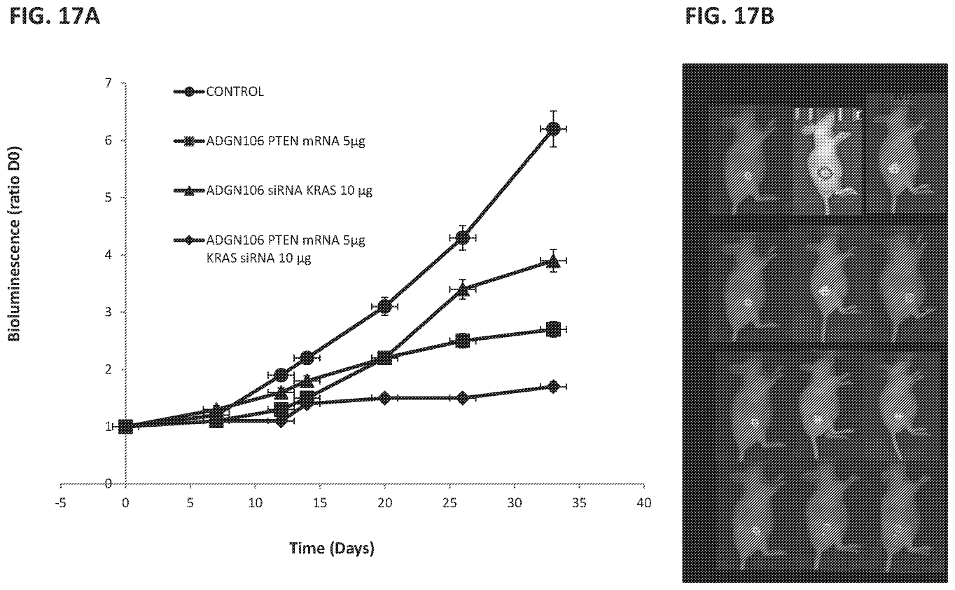

As shown in FIGS. 17A and 17B, in the control group, tumor size increased by 4.6 folds over a period of 26 days. IV administration of 10 μg of PTEN mRNA using ADGN-106 reduced tumor growth by 57% (2.0 folds). Administration of 10 μg KRAS siRNA in complex with ADGN-106 inhibited tumor growth by 35% (3.0 folds increase). Combining mRNA PTEN (5 μg) with KRAS siRNA (10 μg) inhibited tumor growth by 68% (1.5 folds increase). The results show that ADGN-106 mediated in vivo delivery of both mRNA and siRNA and demonstrated a synergy between mRNA PTEN and siRNA KRAS in inhibiting pancreatic tumor progression in vivo. These data indicate that a tumor suppressor and oncogene combination therapy may be useful in cancer treatment.

### Example 5: In Vivo ADGN Mediated Factor VIII mRNA Delivery Evaluation

Materials:

Factor VIII mRNA was obtained using HiScribe™ T7 ARCA mRNA Kit (New England Biolab) mRNA was synthesized using linear vector PGL-FACTOR VIII as DNA template (Addgene 13039) and purified by phenol:chloroform extraction. Synthesized mRNA was purified by LiCl precipitation, phenol:chloroform extraction followed by ethanol precipitation, then quantified by UV Light Absorbance. RNA concentration was determined by measuring the ultraviolet light absorbance at 260 nm. 18 μg of capped mRNA was obtained using 1 μg of DNA template and stored at −20° C.

Factor VIII siRNA were obtained from Thermo Fisher, targeting mouse Factor VIII gene location 2912, siRNA (sense) GATGAGGCTATTCATGATGATT-3′ (SEQ ID NO: 85).

The following ADGN peptide sequences were used:

ADGN-100:
(SEQ ID NO: 79)
βAKWRSAGWRWRLWRVRSWSR,
and
ADGN-105:
(SEQ ID NO: 77)
βALWRALWRLWRSLWRLLWKA.

Results

Factor VIII (FVIII) is an essential blood-clotting protein, also known as anti-hemophilic factor (AHF). In humans, factor VIII is encoded by the F8 gene. Defects in this gene result in hemophilia A, a recessive X-linked coagulation disorder. Factor VIII is produced in liver sinusoidal cells and endothelial cells outside of the liver throughout the body. Haemophilia A is a rare X-linked recessive bleeding disorder that is caused by the deficiency or absence of FVIII. Several approaches have been proposed to restore Factor VIII level in haemophilia patients including the use of viral vectors. Herein we investigated the potency of the ADGN-100 to deliver factor VIII mRNA in the liver in order to restore Factor VIII level. We first established mice with low Factor VIII level by targeting endogenous, hepatocyte-expressed blood clotting factor VIII (FVIII) in Balb C mice using siRNA targeting FVIII (siFVIII).

In order to obtain a transient knockout of Factor VIII expression in the liver, at Day 0, mice from group G1, G2, G3, G4, received IV injection of 100 μl ADGN-100/siFVIII, complex in saline buffer (90 mM NaCl) (siFVIII dose 1.0 mg/kg, 10 μg). Control mice from group N1 received IV injection of 100 μl containing Naked siRNA siFVIII 10 μg and Untreated Control mice from group C1 received 100 μl of saline buffer. After 10 days, animals were injected with ADGN-100/siFVIII, and divided in 4 different groups (3 animals per group) with further treatment as follows:

G1: no treatment

G2: mRNA/ADGN-100 (10 μg) single injection

G3: mRNA/ADGN-106 (10 μg) single injection

G4: Naked mRNA (10 μg) single injection

Factor VIII level was monitored using Factor VIII ELISA kit on blood samples at different time points from Day 0 to Day 50. At Day 50 animals were re-injected with siRNA complexes and at Day 60 animals were re-injected with mRNA complexes as described in groups G1 to G4. Then Factor VIII level was monitored using Factor VIII Elisa kit on blood samples until Day 90. Animal weight was measured every 3 to 5 days.

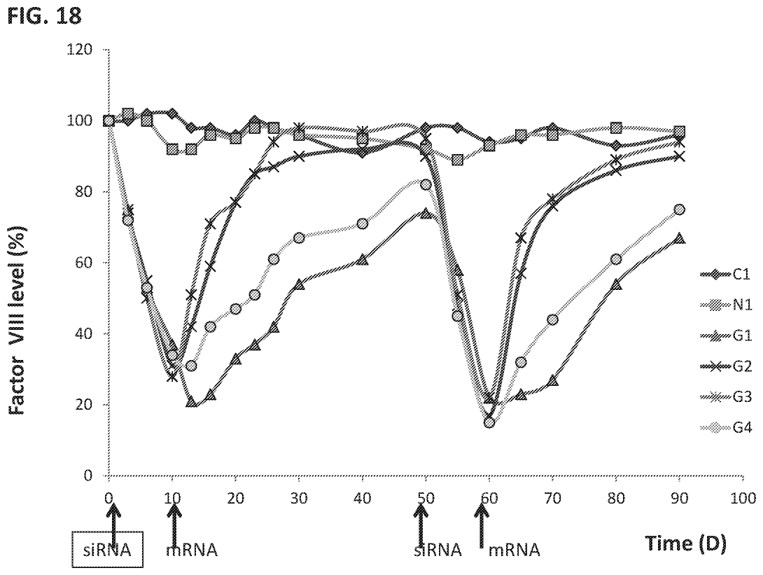

As shown in FIG. 18, ADGN-100 mediated siRNA delivery induced a major down regulation of Factor VIII protein level in the plasma. siRNA effect was observed starting on Day 2 and reached a 72% knockdown at day 10. ADGN-100 and ADGN-106 mediated FVIII mRNA delivery resulted in a rapid liver expression and recovery of the Factor VIII level in the plasma. Total recovery of FVIII was obtained 10-12 days after mRNA administration. In contrast, in the control with no treatment (Group G1) or IV administration of naked FVIII mRNA (Group G4) had only 65% and 72% FVIII recovery at 50 days.

The retreatment at 50 days with the ADGN-siFVIII resulted is a similar knockdown of FVIII as the first treatment. Retreatment at 60 days with the ADGN-100/mRNA and ADGN-106/mRNA showed a very similar pattern of recovery of FVIII levels as the initial treatment. We demonstrated that treatment can be repeated with the same efficiency.

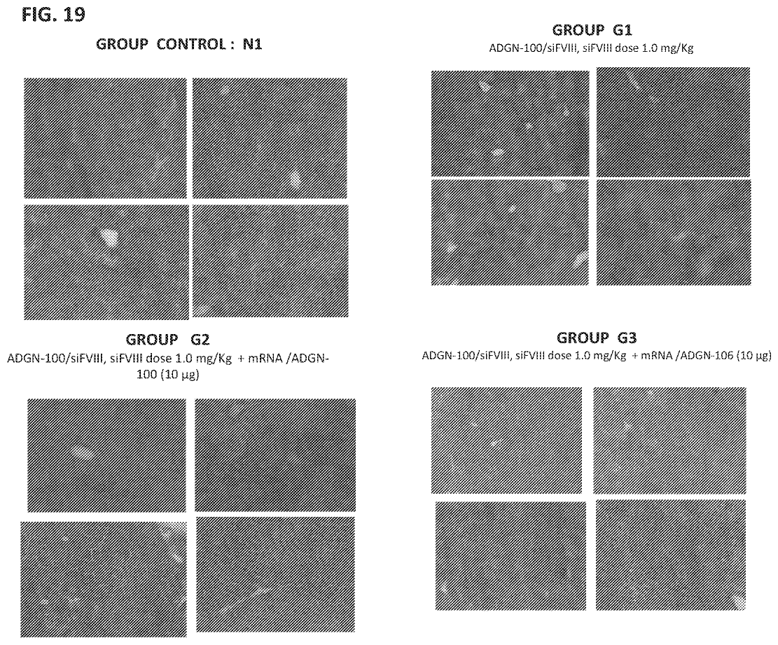

No ADGN associated toxicity or modification in animal weight were detected. Moreover, Liver histological analysis (FIG. 19) showed no inflammation or chronic alteration at Day 90 after two successive treatments. Therefore, these results demonstrate that ADGN-100 and ADGN-106 peptides can be used as potent non-toxic methods for in vivo administration of mRNA and for the correction of numerous genetic disorders in vivo.

### Example 6: Peptide-Mediated CRISPR Complex Delivery in PANC-1 Tumor Model

Materials:

Lipofectamine 2000, RNAiMAX, TranscriptAid T7 transcription kit, MEGAclear transcription Clean Up kit, GeneArt Genomic Cleavage Detection kit, and Platinum Green Hot Start PCR mix were obtained from Thermo Fisher life Science (France). All oligonucleotides were obtained from Eurogentec (Belgium).

Luciferase gRNA was obtained by in vitro transcription using an sgRNA expression plasmid (Addgene #74190, plasmid pLCKO_Luciferase_sgRNA) according to Hart, T., et al. (2015). Cell, 163(6), 1515-1526. Luciferase target site: ACAACTITACCGACCGCGCC (SEQ ID NO: 82). RNAs were obtained by in vitro transcription. Generation of sgRNA was performed using a generic sgRNA expression plasmid containing a T7 promoter adapter sequence as template for a PCR product, which can be in vitro transcribed. Linear DNA fragments containing the T7 promoter binding site followed by the 20-bp sgRNA target sequence were transcribed in vitro using TranscriptAid T7 high Yield transcription Kit (Thermo Fisher life science, France) following the manufacturer's instructions. In vitro transcribed gRNAs were precipitated with ethanol and further purified using MEGAclear transcription clean up Kit (Thermo Fisher life Science). Stock solutions of sgRNAs were solubilized in water, quantified by UV absorbance and stored at −80° C.

CAS9 mRNA was obtained from ThermoFisher as a polyadenylated and capped form.

The following peptide sequences were used:

ADGN-106:
(SEQ ID NO: 77)
βALWRALWRLWRSLWRLLWKA,
and
ADGN-100:
(SEQ ID NO: 79)
βAKWRSAGWRWRLWRVRSWSR.

Cell lines included PANC-1 pancreatic cancer cells expressing Luc2 and ovarian cancer cells SKOV3 expressing Luc2.

ADGN peptide/mRNA/gRNA particles were prepared at a 20:1 molar ratio of ADGN-Peptide/nucleic acid. Premixed CAS9 mRNA/gRNA (5 μg/15 μg) were mixed with ADGN-100 at a 20:1 molar ratio (peptide to complex) and incubated for 30 min at 37° C. Prior to IV administration, complexes were diluted in sucrose 5% solution or saline physiological buffer (90 mM NaCl final concentration).

Results

We have evaluated the potency of ADGN-100 peptide to deliver CRISPR complex (mRNA CAS9/gRNA Luc) targeting luciferase on two cancer cell types expressing Luciferase. Cells were cultured in Dulbecco's Modified Eagle's Medium (DMEM), supplemented with 2 mM glutamine, 1% antibiotics (streptomycin 10,000 μg/mL, penicillin, 10,000 IU/mL) and 10% (w/v) foetal calf serum (FCS), at 37° C. in a humidified atmosphere containing 5% CO₂. 24-well plates seeded with 150,000 cells the day prior to transfection were grown to 50-60% confluence and set up to be at about 70% confluent at the time of transfection.

Before transfection, cells were washed twice with DMEM. Cells were then overlaid with 0.2 ml of ADGN-100/CAS9 mRNA/gRNA Luc (0.2 μg/2 μg or 0.5 μg/5 μg), mixed gently, and incubated for 10 min at 37° C. 0.4 mL of fresh DMEM was added and cells were incubated for 20 min at 37° C. 2 mL of complete DMEM containing 15% FCS Was then added in order to reach a final FCS concentration of 10%, without removing the overlay of ADGN-peptide/cargo complexes.

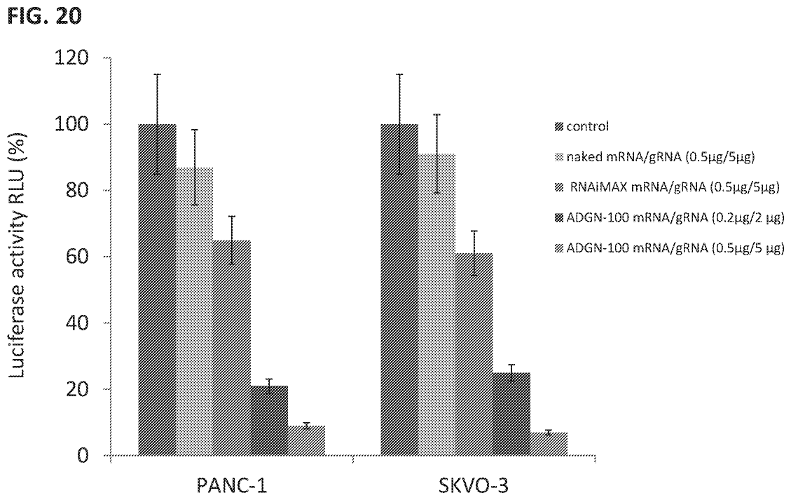

Cells were returned to the incubator (37° C., 5% CO₂) and assayed at 48 hours post transfection for luciferase expression. Results are reported in FIG. 20 as percentage of RLU (luminescence) relative to untreated cells. Controls included cells treated with vehicle, cells treated with naked CAS9 mRNA/gRNA (0.5 μg/5 μg), and cells treated with RNAiMAX/CAS9 mRNA/gRNA (0.5 μg/5 μg) complex. As shown in FIG. 20, in both cell lines ADGN-100 mediated delivery of CRISPR complexes and induced a major reduction in luciferase expression/KD by 80% and 92% for ADGN-100:CAS9 mRNA/gRNA Luc 0.2 μg/2 μg and ADGN-100:CAS9 mRNA-gRNA Luc 0.5 μg/5 μg, respectively. In contrast no change in luciferase level was observed with naked CAS9 mRNA/gRNA (0.5 μg/5 μg). A KD of about 40% was obtained using RNAiMAX, a lipid-based delivery method. The data suggest that ADGN-100 promotes delivery of active CRISPR complex in cancer cells.

We next evaluated the potency of ADGN-100 peptide to deliver CRISPR complex (mRNA CAS9/gRNA Luc) targeting luciferase in vivo in a pancreas tumor mouse model. Female nude mice at 6-weeks of age were implanted in the pancreas with human pancreatic carcinoma cell lines (Panc1-Luc) (20×10⁶ cells in 200 μl PBS). The animals were kept under pathogen-free conditions and fed and watered ad libitum, in cages of 2 to 4 animals (in compliance with recommended area surface/animal), in a dedicated room with a 12 h/12 h light/dark cycle at a constant temperature of 22° C. A period of 3 weeks was allowed for tumor development before the beginning of the experiments.

Two groups of mice (2 animals per group) were used: control group IV tail vein injected with saline solution and ADGN/CRISPR group injected with ADGN-100/5 μg CAS9 mRNA/15 μg Luc gRNA. Animals were injected at Day 0, Day 7, D14, and D20. Tumor size and downregulation of Luc expression in the tumor were evaluated by bioluminescence imaging. Bioluminescence imaging was performed at day 0, 7, 14, 20, and 28. At Day 33, animals were sacrificed and tumors were harvested.

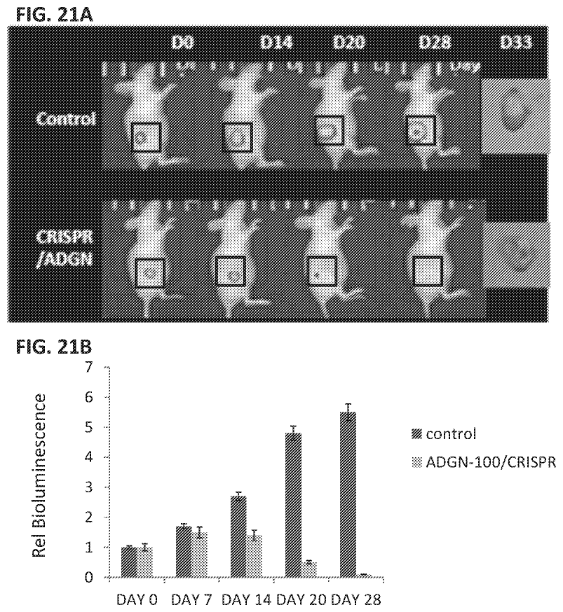

As shown in FIGS. 21A and 21B, IV administration of ADGN-100/CRISPR complex targeting luciferase had no effect on tumor growth, as in both group tumor size increased by 5.5-fold over a period of 33 days (FIG. 21A). In contrast, IV administration of ADGN-100/CRISPR complex dramatically reduced the level of luciferase expression in the tumors (FIG. 21B). The results demonstrate that the ADGN peptides mediated efficient mRNA CAS9/gRNA delivery in the tumor leading to negligible levels of luciferase after 3 administrations of the complexes.

### Example 7: Intracoronary Local Drug Delivery

Healing of an infarct after a myocardial infarction can be problematic and restoration of heart muscle function is often incomplete. In order to stimulate healing of an infarct, local administration of VEGF may increase the formation of new blood vessels to the site of damage. It may be beneficial to administer mRNA encoding VEGF directly to the region of the infarct. To test the ability of local delivery, mRNA encoding beta-galactosidase (β-gal) were complexed with the ADGN peptides to form the peptide/mRNA complexes. Using a catheter suitable for local drug delivery, such as a porous balloon catheter (Scimed or Atrium) or a microinfusion catheter (e.g., Bullfrog, Mercator Medsystems), the peptide/b-gal mRNA complexes (mRNA dose 10 μg-100 μg) were contacted with or injected into the coronary artery wall of New Zealand white rabbits or mini-pigs. 48 hours after the infusion experiments, the heart and the appropriate arterial segments were removed and fixed in formalin solution (4% vol/vol) for CLSM or glutaraldehyde (2.7% vo/vol) in phosphate buffered saline (PBS) 0.1M, pH 7 for transmission electron microscopy or appropriately prepared for visualization of the R-gal stain. The presence of staining was indication that the mRNA was effectively transfected and translated into protein.

Similar local administration of desired mRNA can be achieved in other locations in the body using appropriate infusion devices that are currently available.

### Example 8: Simultaneous In Vivo PTEN Rescue and KRAS Silencing in Cancer Therapy

Methods: ADGN technology is based on short amphipathic peptides that form stable neutral nanoparticles through non-covalent electrostatic and hydrophobic interactions with a large panel of nucleic acids. Self-assembled ADGN/nucleic acid nanoparticles remain stable over time in serum and plasma conditions. ADGN peptide complexes with wildtype PTEN mRNA or siRNA targeting KRAS_{G12}D were evaluated on pancreas (PANC1), ovarian (SKOV3), prostate (PC3) and glioblastoma (U25) cell lines. PTEN, KRAS and AKT phosphorylation were evaluated by western blot. Cell proliferation, cell cycle and apoptosis activation were determined by flow cytometry and Tunel assay. In-vivo Efficacy of IV-administered ADGN-peptides complexed with PTEN mRNA (0.25 mg/kg) and/or KRAS siRNA (0.5 mg/kg) was tested in PANC1-LUC mouse xenografts. Specifically, nude mice 6-weeks of age were injected with Human pancreatic carcinoma cell lines (Panc1-Luc) and were allowed for tumor development. Then mice were treated every 7 days over 4 weeks as described in the following groups in Table 1. Cytokine responses to ADGN-nucleic acid complexes were measured in blood samples using Luminex cytokine 20 plex.

TABLE 3
Treatment groups

| | Material | Dosing | | Route | Frequency |
|---|---|---|---|---|---|
| G1 | Saline | 0 | | IV | 1x weekly |
| G2 | Naked PTEN mRNA | 0.25 | mg/kg | IV | 1x weekly |
| G3 | ADGN/PTEN mRNA | 0.25 | mg/kg | IV | 1x weekly |
| G4 | Naked siRNA KRAS | 0.5 | mg/kg | IV | 1x weekly |
| G5 | ADGN/siRNA KRAS | 0.5 | mg/kg | IV | 1x weekly |
| G6 | ADGN/PTEN mRNA/ | 0.25 | mg/kg | IV | 1x weekly |
| | KRAS siRNA | 0.5 | mg/kg | | |

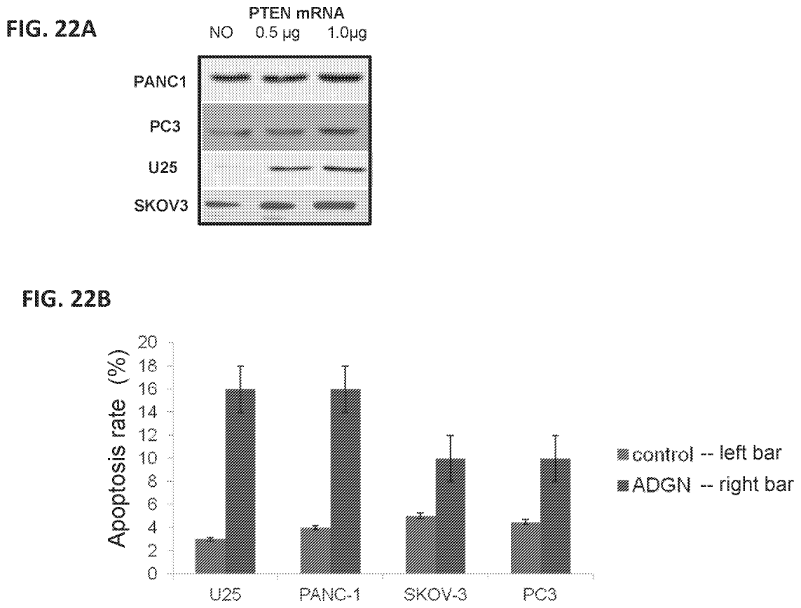

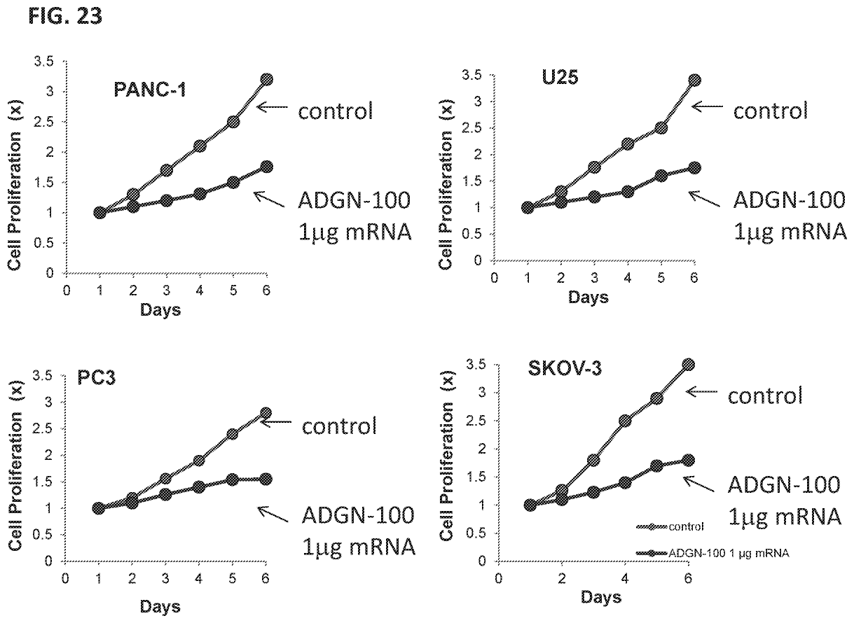

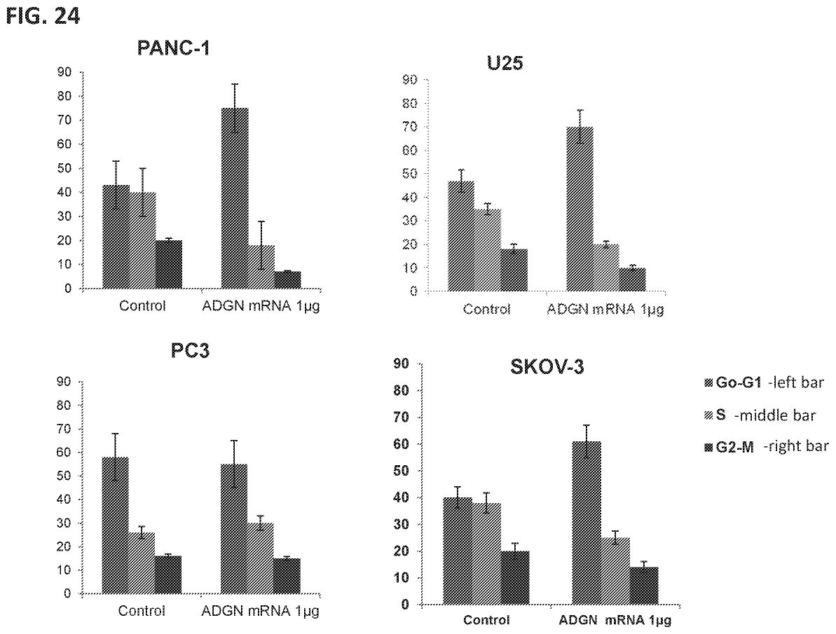

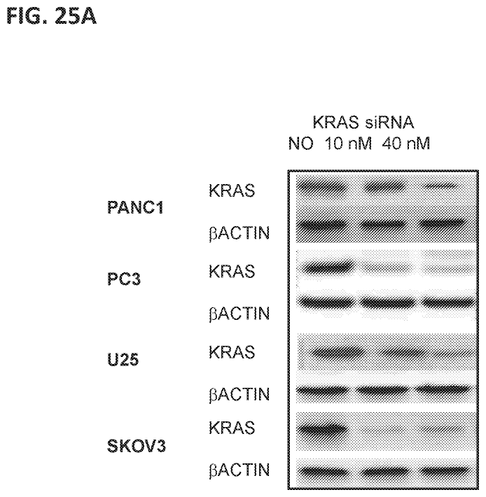

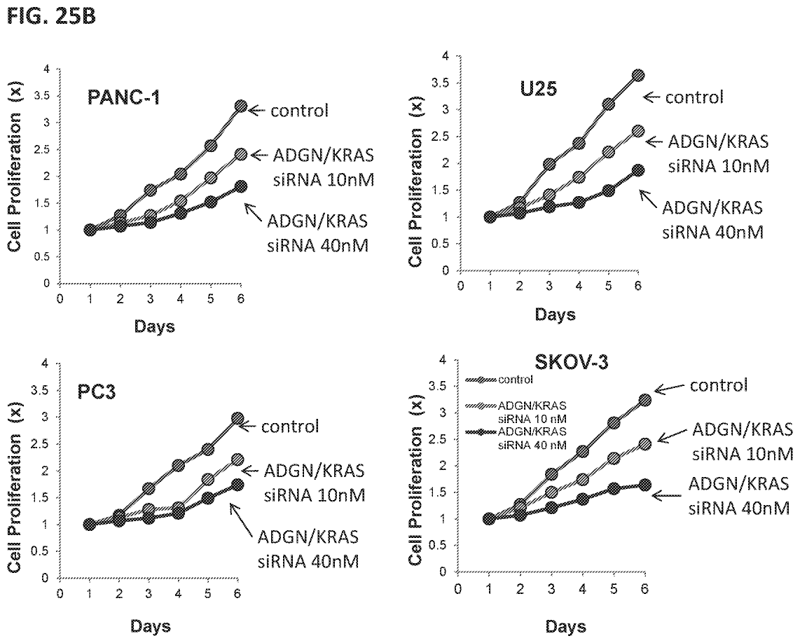

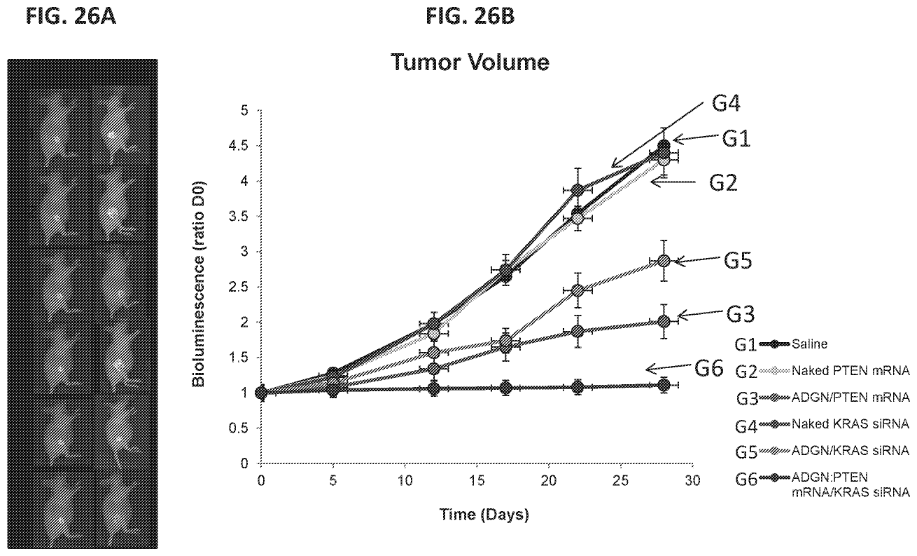

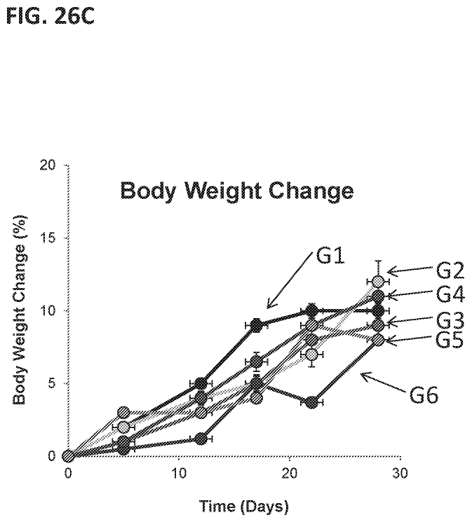

Results: ADGN-mediated delivery of wildtype PTEN mRNA rescued PTEN function in the cell-lines tested. The cells transfected with wildtype PTEN mRNA restored PTEN, which resulted in ˜90% reduction of cell proliferation, 3-8 fold activation of cell apoptosis, reduction of AKT phosphorylation and cell cycle arrest in G1. See FIGS. 22A-B, 23, and 24. ADGN-mediated delivery of siRNA targeting KRAS, induced knock-down of KRAS expression by 60-70% in the cell-lines and significantly reduced viability by 50-70%. See FIGS. 25A and 25B. ADGN/PTEN mRNA and ADGN/KRAS siRNA showed a significant anti-tumor effect compared to their naked forms and saline control. ADGN-nanoparticles containing wildtype PTEN mRNA resulted in tumor growth inhibition (TGI) of 80% (p<0.0001 ANOVA). See FIGS. 26A and 26B. ADGN-nanoparticles containing siRNA KRAS resulted in a TGI of 45-50% (p<0.000l ANOVA). Combination mRNA PTEN and siRNAs KRAS ADON nanoparticles, resulted in a TGI of 90% in PANC1 xenografts (p<0.0001, ANOVA) and also slowed development of distant metastates. See FIGS. 26A and 26B, and data not shown. All treatments were tolerated, with no significant body weight loss in any group. See FIG. 26C. No nonspecific cytokine response was observed following administration of ADGN-nucleic acid complexes.

Conclusions: ADGN-peptide nanoparticles efficiently promote mRNA PTEN and/or siRNA KRAS delivery both in vitro and in vivo. ADGN-peptides complexed with mRNA and/or siRNA were effective in targeting mutated PTEN and KRAS both invitro and invivo. Furthermore, combination of mRNA PTEN rescue with siRNA KRAS knockdown significantly inhibits tumor growth. This suggests a new strategy for simultaneously targeting both tumor suppressors and oncogenes.

### Example 9: Peptide Mediated p53 Tumor Suppressor mRNA Delivery in Tumor Cells

Materials

P53 mRNA: P53 mRNA was obtained using HiScribe™ T7 ARCA mRNA Kit (New England Biolab). mRNA was synthesized using linear vector as DNA template (Addgene Plasmid #24859) and purified by phenol:chloroform extraction. Synthesized mRNA was purified by LiCl precipitation, phenol:chloroform extraction followed by ethanol precipitation, then quantified by UV Light Absorbance. RNA concentration was determined by measuring the ultraviolet light absorbance at 260 nm. 18 μg of capped mRNA was obtained using 1 μg of DNA template and stored at −20° C. For in vivo studies CAS9 mRNA was obtained from ThermoFisher as a polyadenylated and Capped form.

ADGN Peptides: The following peptide sequences were used.

ADGN-106:
(SEQ ID NO: 77)
βALWRALWRLWRSLWRLLWKA
ADGN-100:
(SEQ ID NO: 79)
βAKWRSAGWRWRLWRVRSWSR

Cell lines: Pancreas cancer cells PANC-1. Prostate cancer cells PC3, ovarian cancer cells SKOV3 and human fibroblast HS68 were obtained from ATCC.

Methods

Complex formation with mRNA. The following protocols were used for the transfection of 2-5 10⁶ Cells or Cells at confluency of about 70-800% cultured in 24 well plates. ADGN peptide/mRNA particles were prepared at a 20:1 molar ratio of ADGN-Peptide/mRNA using two doses of mRNA (0.5 μg and 1.0 μg). P53 mRNA (0.5 or 1.0 μg) was diluted in 20 μl of sterile water (GIBCO) at room temperature. 10 μl Final Peptide Solution was added for 0.5 μg of mRNA or 20 μl Final Peptide Solution was added for 1 μg of mRNA to obtain a total volume of 30 μl or 40 μl, respectively. The volume was adjusted to 100 μl with sterile water and the soluture was mixed gently with vortex 1 min low speed and incubated 30 min at room temperature. Just before transfection, the volume was made up to 200 μl by adding sterile water containing 5% sucrose, and the solution was mixed gently with vortex 1 min low speed and incubate 5 min at 37° C. and then was proceeded to cell transfection or in vivo administration.

Transfection protocol. Protocol is reported for 24 well plate format. The volume could be optimized for larger volumes and different plate formats Cells were cultured in Dulbecco's Modified Eagle's Medium (DMEM), supplemented with 2 mM glutamine, 1% antibiotics (streptomycin 10,000 μg/mL, penicillin, 10,000 IU/mL) and 10% (w/v) foetal calf serum (FCS), at 37° C. in a humidified atmosphere containing 5% CO2. 24 well plates seeded with 150,000 cells the day prior to transfection were grown to 50-60% confluence and set up to be at about 70% confluences at the time of transfection.

Before transfection, cells were washed twice with DMEM. Cells were then overlaid with 0.2 ml of complex solution, mixed gently, and incubated for 10 min at 37° C. 0.4 mL of fresh DMEM, were added and cells were incubated for 20 min at 37° C. 2 mL of complete OPTiMEM (high glucose) or DMEM containing 15% FCS were then added in order to reach a final FCS concentration of 10%, without removing the overlay of ADGN-peptide/Cargo complexes.

Cells were returned to the incubator (37° C., 5% CO2) and assayed at 72 hours post transfection. The level of P53 expression was analyzed by western blot using P53 WT monoclonal antibody (p53 Antibody #9282 Cell Signaling technology or DO SC126 Santa Cruz), the impact of P53 WT expression is monitored by proliferation assays using an MTT assay and apoptosis/cell cycle progression using a flow cytometry assay. Cell apoptosis rate (expressed as a percentage) and cell cycle stage were measured using a Propidium Iodide (PI) staining kit (Sigma-Aldrich) and APO BrDu kit (Thermofisher).

Results

For all cell types, the level of P53 was evaluated by western blots using p53 antibody (Thermofisher). Image Lab 4.1 software was used to analyze the protein bands and relative protein expression level was normalized with reference to β-actin (Abcam Inc.).

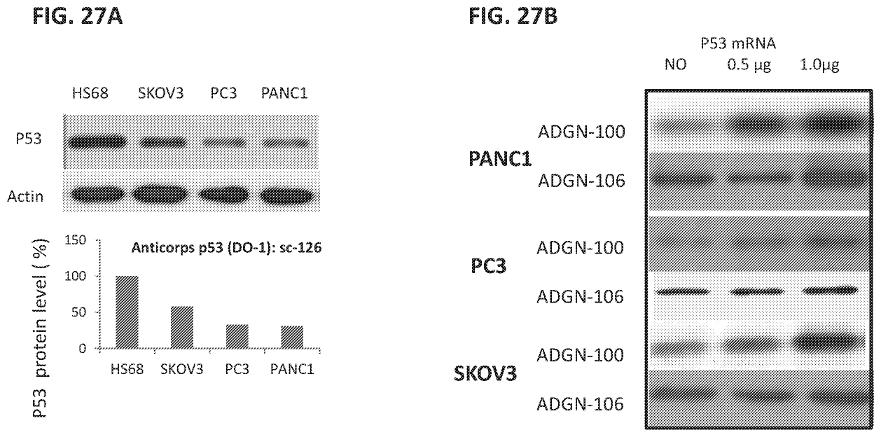

As reported in FIG. 27A, level of wildtype P53 expression was dependent on the cell types. Very low P53 wildtype expression is observed in PANC1 (21%) and PC3 cells (20%). In SKOV3 ovarian carcinoma cells, the level of wildtype P53 corresponds to 51% of that of non-transformed cells (HS-68). Differences in P53 wildtype expression levels were in agreement with previously reported studies; showing that loss in P53 wildtype activity is mainly associated to mutations in P53 gene.

ADGN-100/mRNA and ADGN-106/mRNA complexes were evaluated for cellular delivery of P53WT mRNA in the different cancer cell types. Cells were transfected with 0.5 and 1 μg mRNA in complex with either ADGN-100 and ADGN-106 and level of P53 expression was analyzed by western blot after 48 hrs. As reported in FIG. 27B, in all the cases, ADGN-100 and ADGN-106 promote efficient delivery of P53 WT mRNA leading to P53 protein expression.

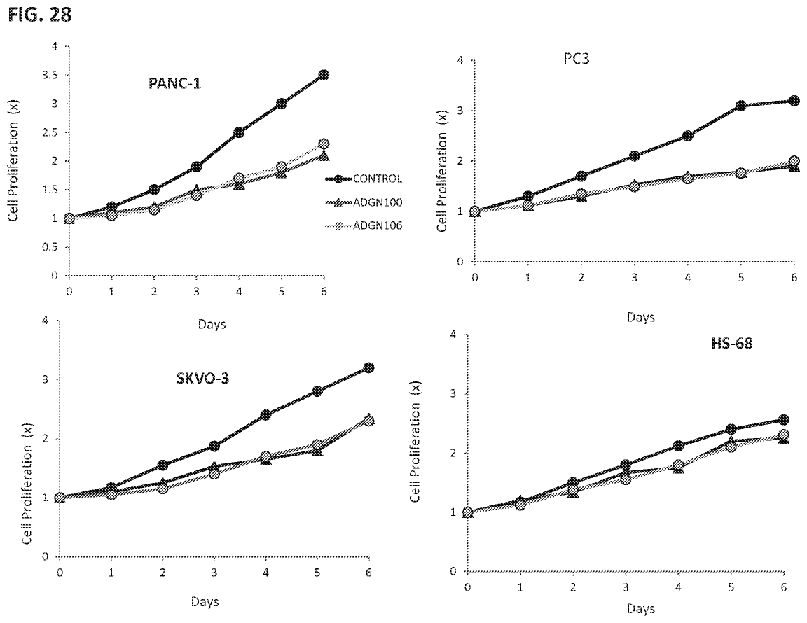

The impact of P53 expression on cancer cell regulation was then evaluated by monitoring cell proliferation over a period of 6 days. As reported in FIG. 28, in all cell types, expression of P53 wildtype mRNA directly correlated to an inhibition of cell proliferation. Cell proliferation inhibition of 71%, 63% and 50% were obtained for PANC1; PC3 and SKOV3 cells, respectively. The reduction in growth curve of cancer cells is marked at Day 6 and indicates that wild type P53 expression strongly decrease the viability of the cells or slow down their proliferation.

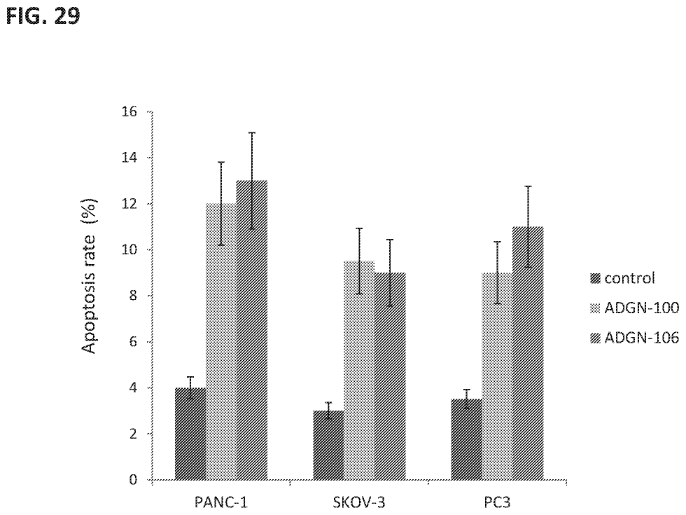

The flow cytometry data as shown in FIG. 29 demonstrated that the rate of cell apoptosis was significantly enhanced in cells expressing wild-type P53, compared with the control cells. Apoptosis level is increase by 5 folds in PANC-1 cells and by 2.8 folds in SKOV-3 and PC3 cells.

The results demonstrated that ADGN-100 and ADGN-106 are potent agents for the delivery of P53 mRNA in cancer cells. ADGN peptides mediated mRNA delivery leads to a large expression of exogenous wild type P53, and rescue P53 functions, by profoundly inhibiting the growth of tumor cells and promoting cellular apoptosis and cell death.

### Example 10: Peptide-Mediated p53 Tumor Suppressor mRNA Delivery In Vivo Pancreas Tumor Xenograft Model

The potency of ADGN peptides (ADGN-100) to deliver P53 mRNA was evaluated in vivo in a pancreas tumor mouse model. Female nude mice 6-weeks of age were implanted in the pancreas with Human pancreatic carcinoma cell lines (Panc1-Luc) (20×10⁶ cells in 200 μl PBS). The animals were kept under pathogen-free conditions and fed and watered ad libitum, in cages of 2 to 4 animals (in compliance with recommended area surface/animal), in a dedicated room with a 12 h/12 h light/dark cycle at a constant temperature of 22° C. A period of 3 weeks was allowed for tumor development before the beginning of the experiments. Three groups of mice were used including 2 control groups G1 &, G2 (3 animals per group) and 1 treatment group (G3) (4 animals per group). The different groups are:

G1: Control Untreated mice (CONTROL) (3 animal/group)
G2: Naked mRNA 10 ug (NAKED) (3 animal/group)
G3: ADGN-100/10 μg P53 mRNA dose 0.5 mg/kg

Animals were injected every 5 days. Mice received IV tail-vein injection of 100 μl ADGN/mRNA complex in saline buffer (90 mM NaCl). Tumor size was evaluated by bioluminescence imaging. Mice received an i.p. injection of 150 μg/g luciferin for non-invasive bioluminescence imaging (IVIS Kinetic; PerkinElmer, Waltham, Mass., USA). Semi-quantitative data of luciferase-positive tumor cell signals were obtained using the manufacturer's software (Living Image; PerkinElmer). Results were expressed as photons/second (photons/s). Bioluminescence imaging was performed at day 0, 7, 14, and 21. Results were then expressed as values relative to day 0.

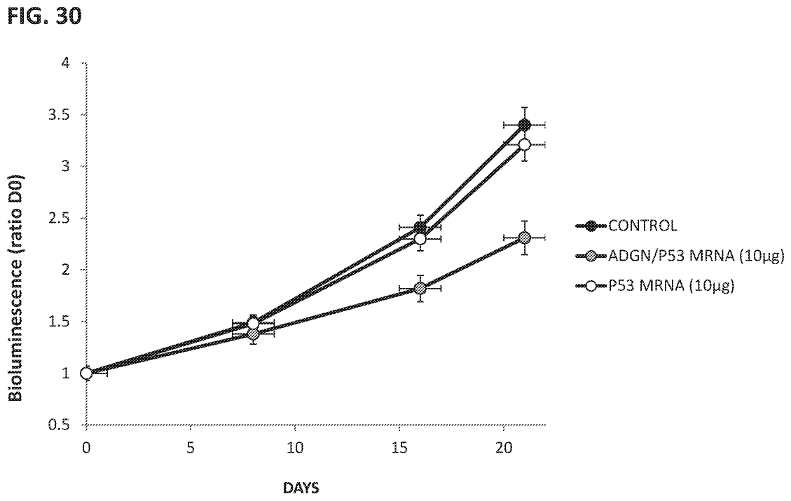

As reported in FIG. 30, in the control group and Naked mRNA group, tumor size increased by 3.4 to 3.6 folds over a period of 21 days. IV administration of 10 μg of P53 mRNA using ADGN-100 significantly inhibits tumor growth, size increasing only by 2.3 fold. The results demonstrated that the ADGN peptide mediated efficient wild type P53 mRNA delivery, restoring P53 function and inhibiting pancreas tumor growth.

### Example 11: Peptide-Mediated Co-Delivery of p53 or PTEN mRNA and KRAS siRNA in Tumor Cells

ADGN-100 and ADGN-106 have been evaluated for co-delivery of PTEN mRNA or P53 mRNA and a cocktail of siRNA targeting several KRAS mutation. The normal KRAS protein performs an essential function in normal tissue signaling, and the mutation of a KRAS gene is an essential step in the development of many cancers. Herein we combined siRNA targeting of the KRAS oncogene together with mRNA to restore PTEN or/and P53 tumor suppressor function. This could offer new perspective for anticancer targeting approach.

The impact of delivery of different KRAS siRNAs that potently inhibit KRAS expression regardless of the specific missense mutation at codons 12 or 61, in cultured cancer cell lines was first validated. The following KRAS siRNA were selected:

siRNA (sense)
(SEQ ID NO: 83)
5′-GUUGGAGCUUGUGGCGUAGTT-3′
and
(anti-sense)
to target specifically the KRAS G12C mutation
(SEQ ID NO: 84)
CUACGCCACCAGCUCCAACTT-3′.
siRNA (sense)
(SEQ ID NO: 86)
5′-GAAGUGCAUACACCGAGACTT-3′
and
(anti-sense)
to target specifically the KRAS Q61K mutation
(SEQ ID NO: 87)
5′-GUCUCGGUGUAGCACUUCTT-3′.
siRNA (sense)
(SEQ ID NO: 88)
5′-GUUGGAGCUGUUGGCGUAGTT-3′
and
(antisense)
to target specifically the KRAS G12D mutation
(SEQ ID NO: 89)
5′-CUACGCCAACAGCUCCAACTT-3′.

KRAS siRNA were first evaluated on cultured cancer cells. Single KRAS siRNAs (10 nM and 40 nM) or combination of KRAS siRNA (5 nM or 20 nM each) were associated to ADGN-106 at 20/1 peptide molar ratio.

Pancreas cancer cells PANC-1, Prostate cancer cells PC3, ovarian cancer cells SKOV3 and human fibroblast HS68 were transfected with ADGN-106: KRAS siRNA complexes, then cell proliferation was measured over a period of 6 days later.

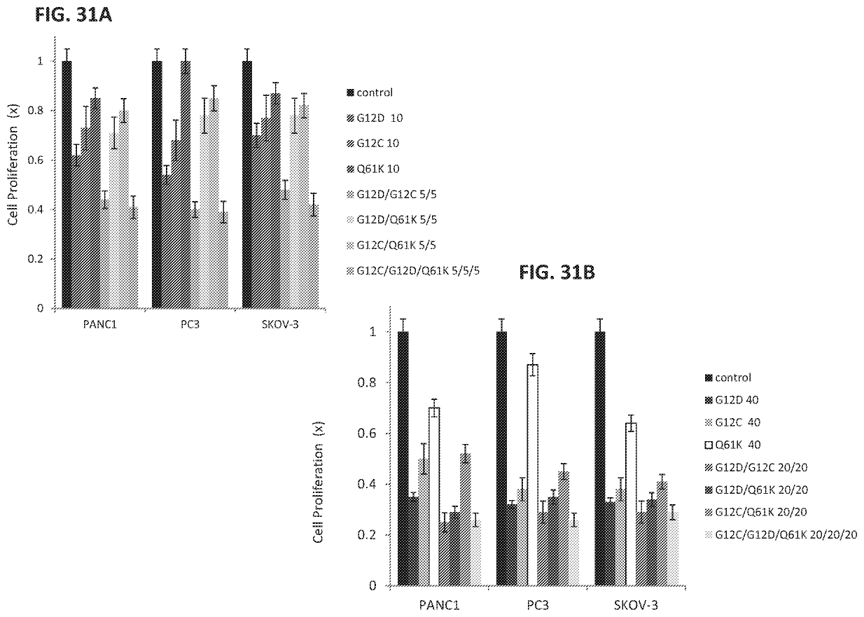

FIGS. 31A and 31B and Table 4 were reported cell proliferation measured 7 days post-transfection using CellTiter-Glo Luminescent Cell Viability Assay (Promega). The results demonstrated that ADGN-106 mediated KRAS siRNA induced a market KD of KRAS mutants, which is directly correlated to a significant decrease in cancer cell proliferation. Combining siRNA targeting G12D and G12C mutants increased proliferation inhibition of all the cancer cell type tested. Combining G12D and G12C siRNA shows a marked potentiation even at low concentration. In contrast no potentiation was obtained by adding siRNA targeting Q61K KRAS mutation. No changes in proliferation were obtained on non-transformed HS68 fibroblast cells.

TABLE 4
Inhibition of Cell proliferation measured 7 days post-transfection (in %).

| CONDITION | PANC1 | PC3 | SKOV-3 | HS-68 |
|---|---|---|---|---|
| None | 0 | 0 | 0 | 0 |
| ADGN/G12D 10 nM | 39 | 52 | 29 | 2 |
| ADGN/G12C 10 nM | 33 | 39 | 26 | 5 |
| ADGN/Q61K 10 nM | 19 | 3 | 17 | 1 |
| ADGN/G12D/G12C 5 nM | 58 | 65 | 53 | 0 |
| ADGN/G12D/Q61K 5 nM | 35 | 29 | 22 | 9 |
| ADGN/G12C/Q61K 5 nM | 26 | 18 | 19 | 2 |
| ADGN/G12C/Q61K/G12D 5 nM | 63 | 65 | 58 | 0 |
| ADGN/G12D 40 nM | 68 | 72 | 69 | 2 |
| ADGN/G12C 40 nM | 48 | 66 | 63 | 1 |
| ADGN/Q61K 40 nM | 32 | 18 | 38 | 1 |
| ADGN/G12D/G12C 20 nM | 78 | 76 | 75 | 5 |
| ADGN/G12D/Q61K 20 nM | 73 | 72 | 71 | 0 |
| ADGN/G12C/Q61K 20 nM | 45 | 58 | 61 | 2 |
| ADGN/G12C/Q61K/G12D 20 nM | 80 | 79 | 76 | 0 |

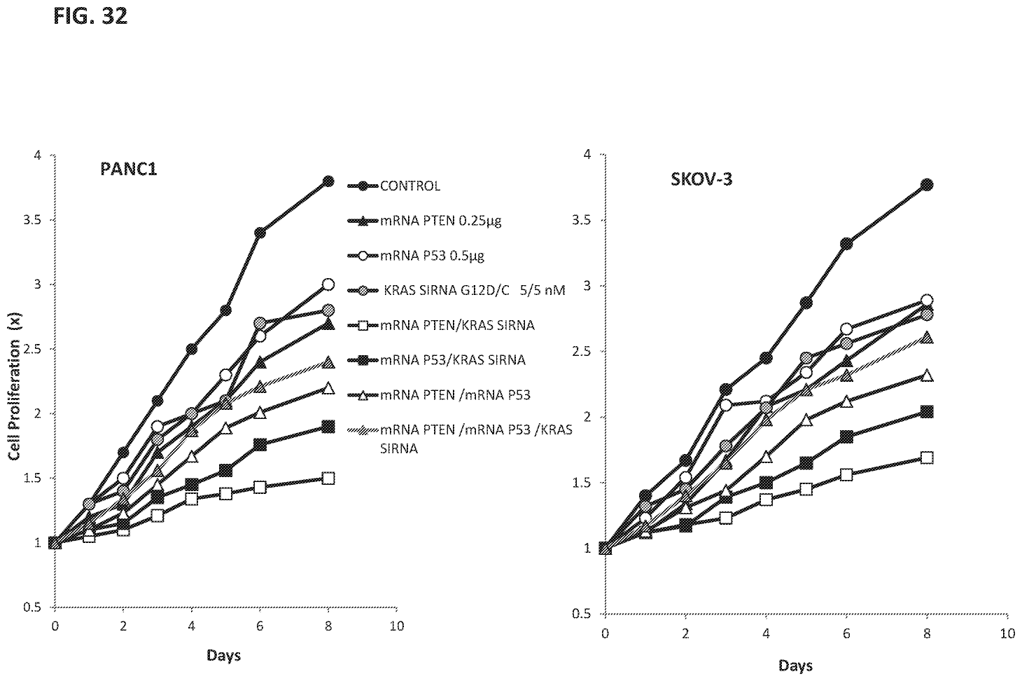

ADGN-100 and ADGN-106 have been evaluated for Co-delivery of PTEN mRNA or P53 mRNA and a cocktail of siRNA targeting several KRAS mutations. PTEN and P53 mRNA were combined with KRAS siRNA and evaluated on PANC1 and SKOV-3 cancer cells. ADGN-100 was used for mRNA PTEN and P53 delivery at 0.25 μg (5.7 nM) and 0.5 μg (11.5 nM) respectively. ADGN 106 was used for KRAS siRNA (G12D/G12C) delivery at 5 nM respectively. Cell proliferation data are reported in FIG. 32 and Table 5. Cell proliferation was measured over a period of 8 days post-transfection using CellTiter-Glo Luminescent Cell Viability Assay (Promega). As reported in FIG. 32 and Table 5, there is an important synergy combining mRNA together with siRNA KRAS. Combining KRAS G12D/G12C with PTEN or P53 mRNA significantly increased inhibition of cancer cell proliferation. An important potentiation was obtained with PTEN mRNA/KRAS siRNA or P53 mRNA/KRAS siRNA.

Combining PTEN mRNA with P53 mRNA lead to a moderate impact on cell proliferation and no significant potentiation was obtained by combining all three PTEN mRNA/P53 mRNA/KRAS siRNA. These data indicates that a tumor suppressor and oncogene combination therapy may be useful in cancer treatment.

TABLE 5
Inhibition of Cell proliferation inhibition measured
7 days post-transfection (in %)

| CONDITIONS | PANC1 | SKOV-3 |
|---|---|---|
| CONTROL | 0 | 0 |
| PTEN mRNA | 45 | 39 |
| P53 mRNA | 34 | 37 |
| KRAS siRNA G12C/GI2D | 39 | 38 |
| PTEN mRNA/KRAS siRNA | 90 | 83 |
| PTEN mRNA/P53 mRNA | 64 | 62 |
| P53 mRNA/KRAS siRNA | 77 | 73 |
| PTEN mRNA/P53 mRNA/KRAS siRNA | 55 | 50 |

### Example 12: Peptide-Mediated KRAS siRNA Delivery in Vivo Pancreas Tumor Xenograft Model

The impact of co-administration of KRAS siRNA targeting mutation at codons 12 was evaluated. Female nude mice 6-weeks of age were injected with Human pancreatic carcinoma cell lines (Panc1-Luc) (20×10⁶ cells in 200 μl PBS). The animals were kept under pathogen-free conditions and fed and watered ad libitum, in cages of 2 to 4 animals (in compliance with recommended area surface/animal), in a dedicated room with a 12h/12h light/dark cycle at a constant temperature of 22° C. A period of 3 weeks was allowed for tumor development before the beginning of the experiments. Animal were treated every 7 days. Mice received IV injection of 100 μl ADGN complexes in saline buffer (90 mM NaCl) as described in the following groups:

G1: Control Untreated mice (CONTROL)
G2: 5 μg siRNA G12C/5 μg siRNA G12D KRAS dose 0.5 mg/kg
G3: ADGN-106/5 μg siRNA G12C/5 μg siRNA G12D KRAS dose 0.5 mg/kg

Tumor size was evaluated by bioluminescence imaging. Mice received an i.p. injection of 150 μg luciferin for non-invasive bioluminescence imaging (IVIS Kinetic; PerkinElmer, Waltham, Mass., USA). Semi-quantitative data of luciferase-positive tumor cell signals were obtained using the manufacturer's software (Living Image; PerkinElmer). Results were expressed as photons/second (photons/s). Bioluminescence imaging was performed at day 0, 7, 14, and 21. Results were then expressed as values relative to day 0.

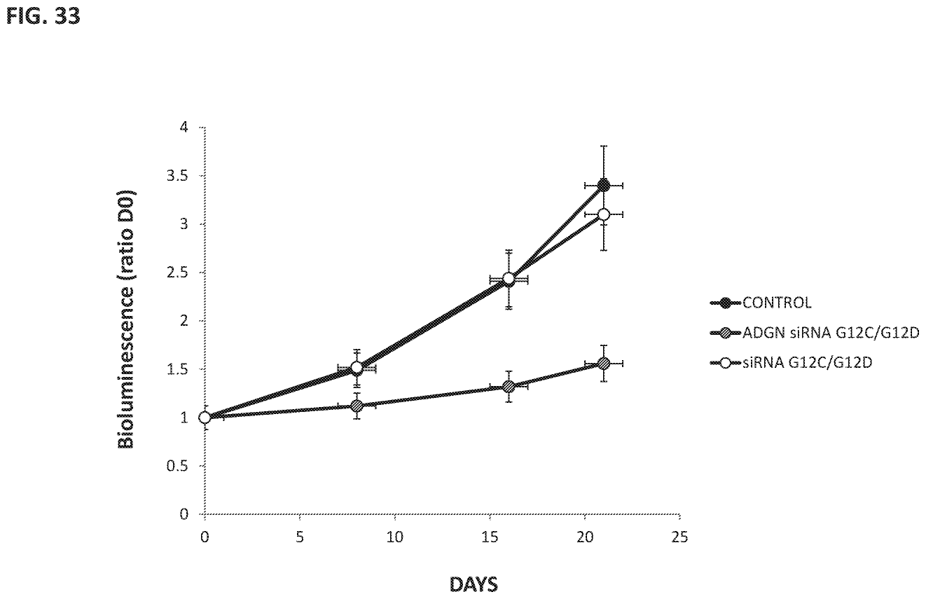

As reported in FIG. 33 in the control group and naked siRNA group, tumor size increased by 3.6 and 3.4 folds over a period of 21 days. IV administration of 5 μg siRNA G12C/5 μg siRNA G12D mRNA using ADGN-106 reduced tumor growth by 77% (1.5 folds). The results show that ADGN-106 mediated in vivo delivery of both KRAS G12C and G12D siRNAs and demonstrated a synergy between the siRNA KRAS in inhibiting pancreatic tumor progression in vivo.

### Example 13: In Vivo ADGN-Mediated Factor VIII mRNA Delivery Evaluation

The potency of the ADGN-100/ADGN 106 to deliver factor VIII mRNA in the liver in order to restore Factor VIll level was evaluated following either IV or subcutaneous (SQ) administrations. Mice with low Factor VIII level were first established by targeting endogenous, hepatocyte-expressed blood clotting factor VIII (FVIII) in Balb C mice using CRISPR targeting FVIII (CRISPR FVIII).

Factor VIII gRNA was designed to disrupt gene expression by causing a double-strandbreak (DSB) in a 5′ constitutive Exon I within the Factor VIII (mouse) gene. Factor VIII gRNA was obtained by in vitro transcription using an sgRNA expression plasmid derived from the Genome-scale CRISPR Knock-Out (GeCKO) v2 library. Factor VIII target site: ATGAAGCACCTGAACACCGT (SEQ ID NO:90). RNAs were obtained by in vitro transcription. Generation of sgRNA was performed using a generic sgRNA expression plasmid containing a T7 promoter adapter sequence as template for a PCR product, which can be in vitro transcribed. Linear DNA fragments containing the T7 promoter binding site followed by the 20-bp sgRNA target sequence were transcribed in vitro using TranscriptAid T7 high Yield transcription Kit (Thermo Fisher life science, France) following the manufacturer's instructions. In vitro transcribed gRNAs were precipitated with ethanol and further purified using MEGAclear transcription clean up Kit (Thermo Fisher life Science). Stock solutions of sgRNAs were solubilized in water, quantified by UV absorbance and stored at −80° C.

CAS9 mRNA was obtained from TermoFisher as a polyadenylated and capped form. Twenty-seven mice were used in this experiment. Control mice from group G1 (3 mice) received IV injection of 100 μl of saline buffer as an untreated control group. In order to obtain a permanent knockout of Factor VIII expression in the liver, at Day 0, the remaining 24 mice from group G2-G8 (3 mice each), received IV injection of 100 μl ADGN-100/CRISPR mRNA/F VIII gRNA, complex in saline buffer (90 mM NaCl) (CRISPR gRNA F VIII dose 0.5 mg/kg). After 10 days, animals injected with ADGN-100/CRISPR F VIII, were divided in 8 different groups (3 animals per group) as following:

G2: no treatment
G3: mRNA/ADGN-100 (20 μg single SQ injection)
G4: mRNA/ADGN-100 (40 μg single SQ injection)
G5: mRNA/ADGN-100 (50 μg single SQ injection)
G6: mRNA/ADGN-106 (20 μg single SQ injection)
G7: mRNA/ADGN-106 (40 μg single SQ injection)
G8: mRNA/ADGN-106 (50 μg single SQ injection)
G9: mRNA/ADGN-100 (10 μg single IV injection)

Factor VIII level was monitored using Factor VIII Elisa kit on blood samples at different time points from Day 0 to Day 45. Animal weight was measured every 3 to 5 days.

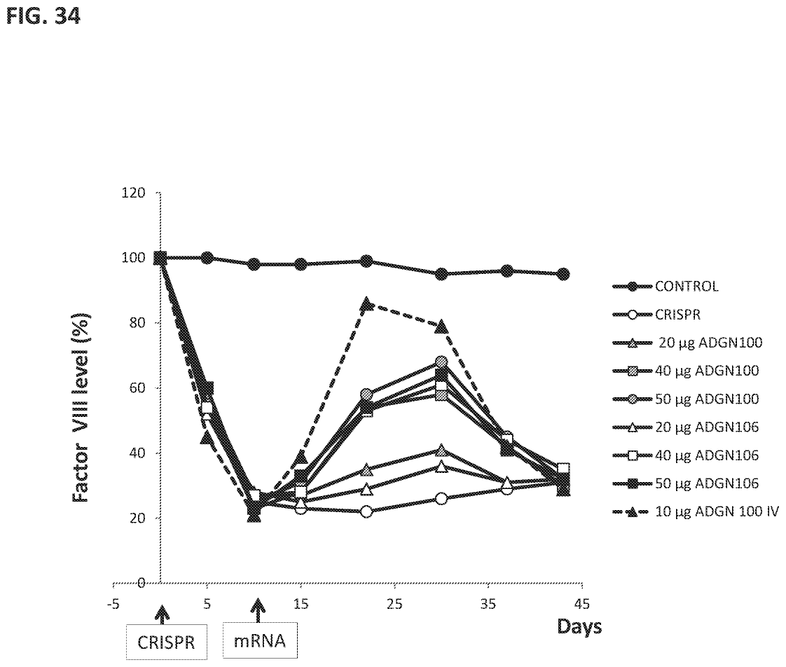

As reported in FIG. 34, ADGN-100 mediated CRISPR F VIII complex delivery induced a major down regulation of Factor VIII protein level in the plasma. CRISPR effect is observed starting on Day 2, to reach 75% knockdown at day 10. ADGN-100 and ADGN-106 mediated FVIII mRNA delivery results in an mRNA dose dependent a rapid liver expression and recovery of the Factor VIII level in the plasma. Recovery of FVIII is obtained 10 days and 15 days after mRNA IV and SQ administration, respectively. As reported in Table 6, the level of FVIII recovery is dependent on the mode of administration and the dose of mRNA injected. mRNA level was maintained during 10 days then decreased rapidly in agreement with Factor VIII in vivo turnover. In contrast, in the control no FVIII recovery was measured at 50 days.

TABLE 6
Level of FVIII recovery 15 days post
mRNA administration (day 30 of experiment)

| | Factor VIII |
|---|---|
| Treatment | recovery (%) |
| CONTROL mice | 100 |
| No | 0 |
| mRNA/ADGN-100 10 μg IV | 81 |
| mRNA/ADGN-100 20 μg SQ | 41 |
| mRNA/ADGN-100 40 μg SQ | 58 |
| mRNA/ADGN-100 50 μg SQ | 68 |
| mRNA/ADGN-106 20 μg SQ | 36 |
| mRNA/ADGN-106 40 μg SQ | 61 |
| mRNA/ADGN-106 50 μg SQ | 64 |

No ADGN associated toxicity or modification in animal weight were detected. Therefore ADGN-100 and ADGN-106 are potent non toxic methods for in vivo IV and SQ administration of mRNA and can be used for the correction of numerous genetic disorders in vivo.

Next, different doses of mRNA that can be used in treatment via multiple SQ administrations to maintain Factor VIII expression level higher than 60% were evaluated. In order to obtain a permanent knockout of Factor VIII expression in the liver, at Day 0, a new set of Balb C mice groups G2-G8 (according to the Table 7 below), received IV injection of 100 μl ADGN-100/CRISPR mRNA/F VIII gRNA, complex in saline buffer (90 mM NaCl) (CRISPR gRNA F VIII dose 0.5 mg/kg). As control mice from group G1 received IV injection of 100 μl of saline buffer as untreated group. After 10 days, animals injected with ADGN-100/CRISPR F VIII, were SQ injected with initial mRNA/ADGN-100 dose (40 μg single SQ injection), At 2 weeks animals were divided in 5 different groups (4 animals per group) and treated by SQ injection with different doses of mRNA complex (according to the table below). FACTOR VIII levels were monitored using either Elisa Chromogenic factor VIII activity assay. Animal weight was measured once a week. Treatment was performed over a period of 3 months.

TABLE 7

| | ADGN- | ADGN- |
|---|---|---|
| | 100/mRNA | 100/mRNA treatment |
| GROUP | initial doses | starting at 2 weeks |
| G1 Untreated | 0 | 0 |
| G2 CRISPR FACTOR VIII | 0 | 0 |
| G3 | 40 μg | 10 μg every 2 weeks |
| G4 | 40 μg | 20 μg every 3 weeks |
| G5 | 40 μg | 30 μg every 4 weeks |
| G6 | 40 μg | 40 μg every 4 weeks |

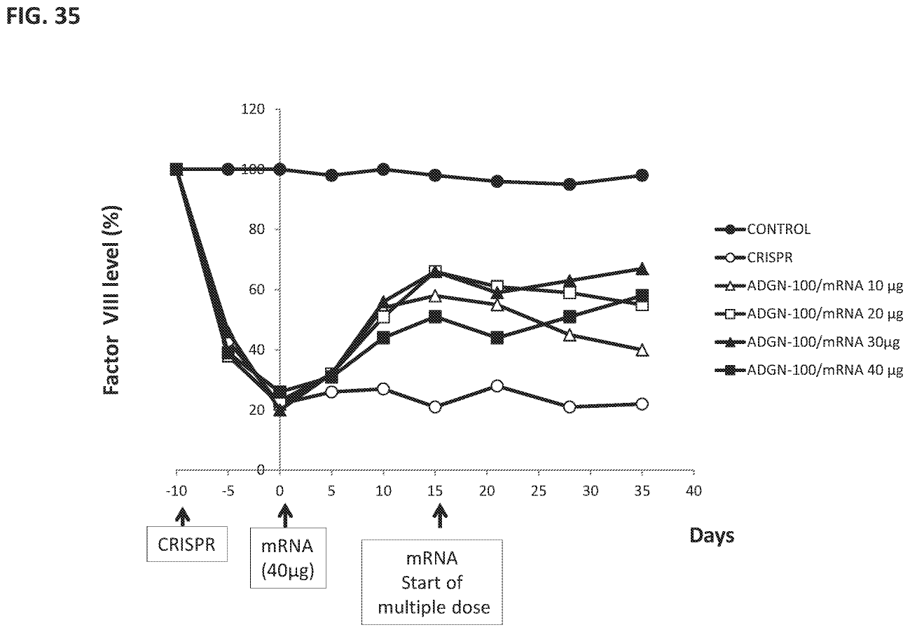

As reported in FIG. 35, ADGN-100 mediated CRISPR F VIII complex delivery induced a major down regulation of Factor Vlll protein level in the plasma to reach 75-78% knockdown at day 10. ADGN-100 mediated 40 μg FVIII mRNA SQ delivery results in a rapid liver expression and recovery of the Factor VIII level up to 60% in the plasma. Recovery of FVIII is obtained 10 days after mRNA administration. SQ injection of 2 g, 30 g and 40 g of mRNA 2 weeks after initial mRNA dose injection, maintained Factor VIII level of expression at 60% In contrast mice SQ injected with 10 μg mRNA show decrease in Factor VIII expression.

### Example 14: Peptide-Mediated Co-Delivery of p53 or PTEN mRNA and KRAS siRNA in Pancreatic and Ovarian Tumors

ADGN-100 and ADGN-106 have been evaluated for co-delivery of PTEN mRNA or P53 mRNA and a cocktail of siRNA targeting several KRAS mutations in vivo in a pancreas and ovarian tumor mouse model. Female nude mice 6-weeks of age were implanted in the pancreas with Human pancreatic carcinoma cell lines (Panc1-Luc) or with ovarian cancer cells (SKOV3-Luc) (20×10⁶ cells in 200 μl PBS).

The animals were kept under pathogen-free conditions and fed and watered ad libitum, in cages of 2 to 4animals (in compliance with recommended area surface/animal), in a dedicated room with a 12 h/12 h light/dark cycle at a constant temperature of 22° C. A period of 3 weeks was allowed for tumor development before the beginning of the experiments.

Fifteen groups of mice (4 animals per group) were used as shown in Table 8.

TABLE 8

| Groups | CONDITIONS | PANC1 | SKOV-3 |
|---|---|---|---|
| 1 | CONTROL | x | x |
| 2 | Naked PTEN mRNA 10 μg (0.5 mg/kg) | x | x |
| 3 | Naked P53 mRNA 10 μg (0.5 mg/kg) | x | x |
| 4 | Naked KRAS siRNA G12C/G12D 10 μg (0.5 mg/kg) | x | x |
| 5 | Naked PTEN mRNA/KRAS siRNA 10 μg (0.5 mg/kg) | x | x |
| 6 | Naked PTEN mRNA/P53 mRNA 10 μg (0.5 mg/kg) | x | x |
| 7 | Naked P53 mRNA/KRAS siRNA 10 μg (0.5 mg/kg) | x | x |
| 8 | Naked PTEN mRNA/P53 mRNA/KRAS siRNA 10 μg (0.5 mg/kg) | x | x |
| 9 | ADGN-100/PTEN mRNA 10 μg (0.5 mg/kg) | x | x |
| 10 | ADGN-100/P53 mRNA 10 μg (0.5 mg/kg) | x | x |
| 11 | ADGN-106/KRAS siRNA G12C/G12D 10 μg (0.5 mg/kg) | x | x |
| 12 | ADGN-100 PTEN mRNA/ADGN-106 KRAS siRNA 10 μg (0.5 mg/kg) | x | x |
| 13 | ADGN-100 PTEN mRNA/ADGN-100 P53 mRNA 10 μg (0.5 mg/kg) | x | x |
| 14 | ADGN-100 P53 mRNA/ADGN-106 KRAS siRNA 10 μg (0.5 mg/kg) | x | x |
| 15 | ADGN-100 PTEN mRNA/ADGN-100 P53 mRNA/ | x | x |
| | ADGN-106 KRAS siRNA 10 μg (0.5 mg/kg) | | |

Animal were injected every 5 days. Mice received IV tail-vein injection of 100 μl ADGN/mRNA or ADGN siRNA complexes in saline buffer (90 mM NaCl). Tumor size was evaluated by bioluminescence imaging. Mice received an i.p. injection of 150 μg/g luciferin for non-invasive bioluminescence imaging (IVIS Kinetic; PerkinElmer, Waltham, Mass., USA). Semi-quantitative data of luciferase-positive tumor cell signals were obtained using the manufacturer's software (Living Image; PerkinElmer). Results were expressed as photons/second (photons/s). Bioluminescence imaging was performed at day 0, 7, 14 and 21. Results were then expressed as values relative to day 0.

A. Peptide-mediated co-delivery of p53 or PTEN mRNA and KRAS siRNA in Pancreatic Tumors

ADGN-100 and ADGN-106 have been evaluated for co-delivery of PTEN mRNA or P53 mRNA and a cocktail of siRNA targeting several KRAS mutations in vivo in a pancreas and ovarian tumor mouse model. Female nude mice 6-weeks of age were implanted in the pancreas with Human pancreatic carcinoma cell lines (Panc1-Luc) (20×10⁶ cells in 200 μl PBS). A period of 3 weeks was allowed for tumor development before the beginning of the experiments.

The animals were kept under pathogen-free conditions and fed and watered ad libitum, in cages of 2 to 4animals (in compliance with recommended area surface/animal), in a dedicated room with a 12 h/12 h light/dark cycle at a constant temperature of 22° C. A period of 3 weeks was allowed for tumor development before the beginning of the experiments.

Six groups of mice were identified Control Untreated mice (G1), mice injected with ADGN-100/PTEN mRNA (0.5 mg/kg) (G2), ADGN-100/P53 mRNA (0.5 mg/kg) (G3), ADGN-100/PTEN mRNA (0.5 mg/kg)/ADGN-106/KRAS SiRNA (0.5 mg/kg) (G4), ADGN-100/PTEN mRNA/P53 mRNA (0.5 mg/kg) (G5) and ADGN-100/P53 mRNA (0.5 mg/kg)/ADGN-106/KRAS siRNA (0.5 mg/kg) (G6). Animal were IV tail-vein injected every 7 days. Tumor size was evaluated by bioluminescence imaging at day 0, 4, 7, 11, 15, 20, 25, 30, 37.

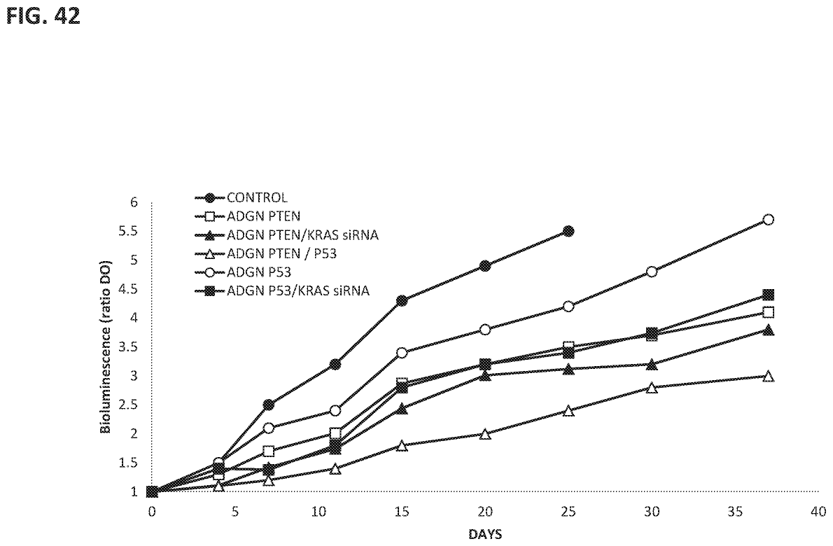

As reported in FIG. 42, in the control group, the tumor size increased by 5.5 fold over a period of 25 days. IV administration of PTEN mRNA (0.5 mg/kg) or P53 mRNA (0.5 mg/kg) using ADGN-100 limited the tumor growth to 3.5 and 4.2 fold, respectively. Co-delivery via IV administration of 10 μg of PTEN mRNA/ADGN-100 and a cocktail of siRNA targeting KRAS (G12D, G12C)/ADGN-106 (5 μg each), limited the tumor growth to 2.9 fold over a period of 25 days and to 3.8 fold over a period of 38 days, which corresponds to 47% inhibition of tumor growth. Co-delivery via IV administration of 10 μg of P53 mRNA/ADGN-100 and a cocktail of siRNA targeting KRAS (G12D, G12C)/ADGN-106 (5 μg each), limited the tumor growth to 3.4 fold over a period of 25 days and to 4.4 fold over a period of 38 days, which corresponds to 38% inhibition of tumor growth. Co-delivery via IV administration of 10 μg of PTEN mRNA/P53 mRNA using ADGN-100, limited the tumor growth to 2.4 fold over a period of 25 days and to 3.0 fold over a period of 38 days, which corresponds to 59% inhibition of tumor growth.

The results demonstrated a synergy between restoring PTEN and P53 function as well as between restoring P53 function and inhibiting KRAS mutation in inhibiting pancreatic tumor progression in vivo. These data indicate that a tumor suppressor and oncogene combination therapy may be useful in cancer treatment.

### Example 15: Peptide-Mediated Co-Delivery of PTEN mRNA and/or KRAS siRNA in Pancreatic Tumors in Combination with Abraxane (Nab-Paclitaxel) and/or Gemcitabine

ADGN-100 and ADGN-106 have been evaluated for co-delivery of PTEN mRNA and a cocktail of siRNA targeting several KRAS mutations (G12D/G12C) in vivo in a pancreas mouse model. Female nude mice 6-weeks of age were implanted in the pancreas with Human pancreatic carcinoma cell lines (Panc1-Luc) (20×10⁶ cells in 200 μl PBS).

The animals were kept under pathogen-free conditions and fed and watered ad libitum, in cages of 2 to 4 animals (in compliance with recommended area surface/animal), in a dedicated room with a 12 h/12 h light/dark cycle at a constant temperature of 22° C. A period of 3 weeks was allowed for tumor development before the beginning of the experiments.

Groups of mice (4 animals per group) were used as shown in Table 9.

TABLE 9

| Group | CONDITIONS |
|---|---|
| 1 | CONTROL |
| 2 | ADGN-100/PTEN mRNA 10 μg (0.5 mg/kg) |
| 3 | ADGN-106 KRAS siRNA 10 μg (0.5 mg/kg) |
| 4 | Abraxane 10 mg/kg |
| 5 | ADGN-100 PTEN mRNA/ADGN-106 KRAS siRNA 10 μg (0.5 mg/kg) |
| 6 | Abraxane (10 mg/kg)/ADGN-106 KRAS siRNA 10 μg (0.5 mg/kg) |
| 7 | Abraxane (10 mg/kg)/ADGN-100 PTEN mRNA/ADGN-106 KRAS |
| | siRNA 10 μg (0.5 mg/kg) |
| 8 | Abraxane (10 mg/kg)/gemcitabine (standard dose)/ |
| | ADGN-100 PTEN mRNA/ADGN-106 KRAS siRNA |
| | 10 μg (0.5 mg/kg) |

Animal were injected every 5 days. Mice received IV tail-vein injection of 100 μl ADGN/mRNA or ADGN/siRNA complexes in saline buffer (90 mM NaCl). Tumor size was evaluated by bioluminescence imaging. Mice received an i.p. injection of 150 μg/g luciferin for non-invasive bioluminescence imaging (IVIS Kinetic; PerkinElmer, Waltham, Mass., USA). Semi-quantitative data of luciferase-positive tumor cell signals were obtained using the manufacturer's software (Living Image; PerkinElmer). Results were expressed as photons/second (photons/s). Bioluminescence imaging was performed at day 0, 7, 14 and 21. Results were then expressed as values relative to day 0.

A. Peptide-Mediated Co-Delivery of PTEN mRNA and/or KRAS siRNA in Pancreatic Tumors in Combination with Abraxane (Nab-Paclitaxel)

ADGN-100 and ADGN-106 have been evaluated for co-delivery of PTEN mRNA and a cocktail of siRNA targeting several KRAS mutations (G12D/G12C) in vivo in a pancreas mouse model. Female nude mice 6-weeks of age were implanted in the pancreas with Human pancreatic carcinoma cell lines (Panc1-Luc) (20×10⁶ cells in 200 μl PBS).

The animals were kept under pathogen-free conditions and fed and watered ad libitum, in cages of 2 to 4 animals (in compliance with recommended area surface/animal), in a dedicated room with a 12h/12h light/dark cycle at a constant temperature of 22° C. A period of 3 weeks was allowed for tumor development before the beginning of the experiments.

Animal were injected once a week at day 0, 7, 14, 21, 28 and 34. Mice received IV injection of 100 μl ADGN-100/PTEN mRNA (10 μg, 0.5 mg/kg) or ADGN-106/KRAS SiRNA G12C & G12D (5 ug/5 μg) complex and/or Abraxane (50 μg paclitaxel, 2.5 mg/kg). Six groups of mice were identified Control Untreated mice (G1), mice injected with ADGN/PTEN mRNA (0.5 mg/kg)/ADGN/KRAS siRNA (0.5 mg/kg) (G2), with Abraxane (2.5 mg/kg) (G3), with Abraxane (2.5 mg/kg) ADGN/PTEN mRNA (0.5 mg/kg) (G4), with Abraxane (2.5 mg/kg) ADGN-106/KRAS SiRNA G12C & G12D (0.5 mg/kg) (G5) or Abraxane (2.5 mg/kg)/ADGN-100/PTEN mRNA (0.5 mg/kg)/ADGN-106/KRAS SiRNA G12C & G12D (0.5 mg/kg) (G6). Animal were IV tail-vein injected every 7 days. Tumor size was evaluated by bioluminescence imaging at day 0, 4, 7, 11, 15, 20, 25, 30, 37.

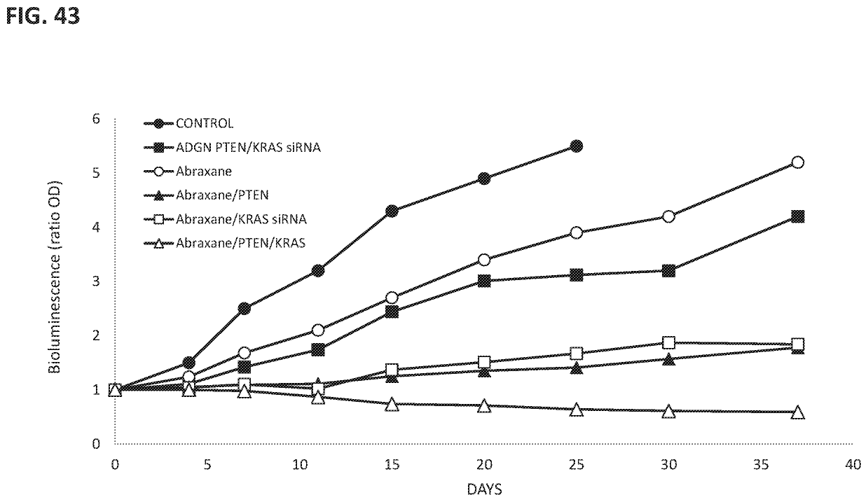

The results are reported in FIG. 43, in the control group, the tumor size increased by 5.4 over a period of 25 days. Co-delivery via IV administration of 10 μg of PTEN mRNA/ADGN-100 and a cocktail of siRNA targeting KRAS (G12D, G12C)/ADGN-106 (5 μg each), limited the tumor growth to 2.9 fold over a period of 25 days and to 4.2 fold over a period of 38 days, which corresponds to 47% inhibition of tumor growth. Administration of Abraxane (50 μg, 2.5 mg/kg) reduced tumor growth to 3.9 fold over a period of 25 days and to 5.2 fold over a period of 38 days, corresponding to 29% inhibition of tumor growth.

Combining Abraxane together with ADGN-100/PTEN mRNA or with ADGN-106/KRAS SiRNA G12C & G12D significantly inhibits tumor growth, by 75% and 70%, respectively. The tumor size increased only by 1.8 fold for a period of 38 days, in both cases. Most strikingly, mice treated with the combination of Abraxane, ADGN-100/PTEN mRNA and ADGN-106/KRAS siRNA G12C & G12D had shrinked tumors as compared to day 0.

### Example 16: ADGN Mediated p53 Tumor Suppressor mRNA Delivery in Human Osteosarcoma Cells

A large percent of all osteogenic sarcomas (OS) harbor p53 alterations that render p53 inactive. The loss of p53 activity may become the oncogenic driver in these tumors. The need for improved therapies is this highly aggressive bone tumor of adolescents and young adults is evidenced by the fact that therapy and outcomes have not changed over the past 20 years. Using the ADGN peptides with p53 mRNA, we can re-express p53 in OS. Fully genetically characterized human OS cell lines, for example, G292, HOS, SaOSs and MG63, all harbor p53 alterations. Each of these cell lines will also be grown as mouse xenografts.

We will test the effect of re-expression of wild-type p53 on the in vitro growth of each of these OS cell lines using real-time imaging. Each cell line will be tested in quadruplicate and we obtain growth curves by assaying real time imaging every four hours, comparing cells expressing the wt p53 and control infected cells. We will then follow these studies in mouse xenograft models treated with ADGN-peptide p53 mRNA complexes.

### Example 17: ADGN Mediated p53 Tumor Suppressor mRNA or eGFP mRNA Delivery in Human Osteosarcoma Cells

ADGN-100/mRNA and ADGN-106/mRNA complexes were evaluated for cellular delivery of P53 WT mRNA or eGFP mRNA in Human Osteosarcoma cell G-292.

A. Delivery of p53 Tumor Suppressor mRNA or eGFP mRNA

Materials

mRNA.

eGFP mRNA was used a positive control of transfection. CleanCap™ EGFP mRNA (5moU) was obtained for Trilink Biotechnology (USA). P53 mRNA was obtained using HiScribe™ T7 ARCA mRNA Kit (New England Biolab). mRNA was synthesized using linear vector as DNA template (Addgene Plasmid #24859) and purified by phenol:chloroform extraction. Synthesized mRNA was purified by LiCl precipitation, phenol:chloroform extraction followed by ethanol precipitation, then quantified by UV Light Absorbance. No specific modification was added to enhance mRNA translation or nuclease stability. RNA concentration was determined by measuring the ultraviolet light absorbance at 260 nm. 18 μg of capped mRNA was obtained using 1 μg of DNA template and stored at −20° C.

ADGN Peptides:

the following peptide sequences were used.

ADGN-106:
(SEQ ID NO: 77)
βALWRALWRLWRSLWRLLWKA
ADGN-100:
(SEQ ID NO: 79)
βAKWRSAGWRWRLWRVRSWSR

Cell lines: Human Osteosarcoma cell G-292, clone A141B1 were obtained from ATCC

Methods. Complex formation with mRNA. The following protocols were used for the transfection of 2-5 10⁶ Cells or Cells at confluency of about 70-80% cultured in 24 well plates. ADGN peptide/mRNA particles were prepared at a 20:1 molar ratio of ADGN-Peptide/mRNA (ADGN-100 or ADGN-106) using three doses of mRNA (0.25 μg, 0.5 μg and 1 μg). P53 mRNA or eGFP mRNA (0.25, 0.5and 1 μg) was diluted in 20 μl of sterile water (GIBCO) at room temperature. 5 μl, 10 μl and 20 μl Final Peptide Solution was added for 0.25 μg, 0.5 μg and 1 μg of mRNA to obtain a total volume of 20 μl or 30 μl, respectively. The volume was adjusted to 50 μl with sterile water and was mixed gently with vortex 1 min low speed and incubated 30 min at room temperature. Just before transfection, the volume was made up to 100 μl by adding sterile water containing 5% sucrose, and the solution was mixed gently with vortex 1 min low speed and incubate 5 min at 37° C. and then was proceeded to cell transfection.

Transfection protocol. Protocol is reported for 24 well plate format. Cells were cultured in Dulbecco's Modified Eagle's Medium (DMEM), supplemented with 2 mM glutamine, 1% antibiotics (streptomycin 10,000 μg/mL, penicillin, 10,000 IU/mL) and 10% (w/v) foetal calf serum (FCS), at 37° C. in a humidified atmosphere containing 5% CO2. 24 well plates seeded with 150,000 cells the day prior to transfection were grown to 50-60% confluence and set up to be at about 70% confluences at the time of transfection. Before transfection, cells were washed twice with DMEM. Cells were then overlaid with 0.1 ml of complex solution, mixed gently, and incubated for 10 min at 37° C. 0.2 mL of fresh DMEM, were added and cells were incubated for 20 min at 37° C. 1 mL of complete DMEM containing 15% FCS were then added in order to reach a final FCS concentration of 10%, without removing the overlay of ADGN-peptide/mRNA complexes.

Cells were returned to the incubator (37° C., 5% C02) and assayed at 2, 4, 5, and 7 days post transfection. Expression and impact of P53 WT expression were monitored by western blots and proliferation assays using an MTT assay and eGFP expression is quantified by flow cytometry assay.

Results

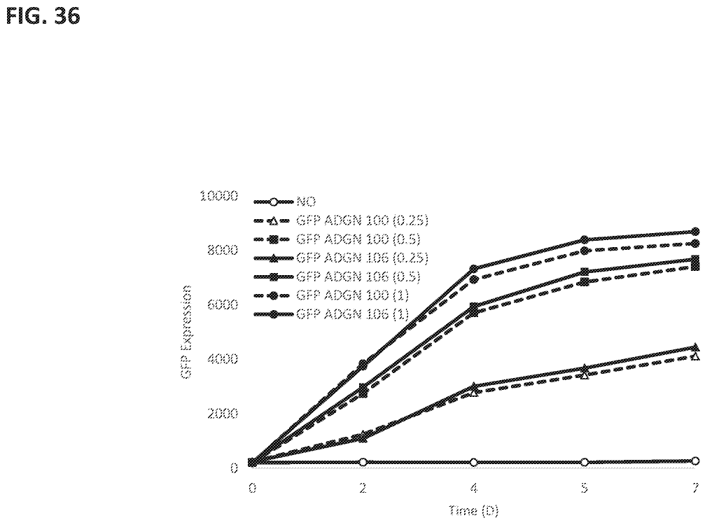

The level of eGFP expression was evaluated by flow cytometry. As reported in FIG. 36, in all the cases, ADGN-100 and ADGN-106 promote efficient delivery of eGFP mRNA leading to eGFP expression in a dose dependent fashion. eGFP mRNA expression was observed at all mRNA concentrations using either ADGN100 or ADGN106 for the delivery. eGFP Expression increased by a factor of 2 between 0.25 μg and 0.5 μg mRNA concentrations and only by 25% between 0.5 and 1 μg mRNA. In all cases, expression of eGFP started at day 2 with a maximal protein expression at day 5 which remains stable at day 7.

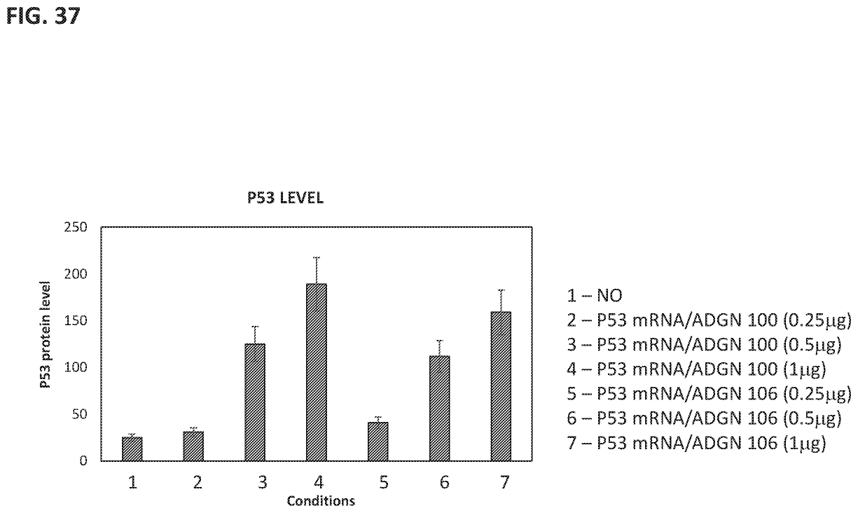

As reported in FIG. 37, Expression of P53 WT was significantly increased using 0.5 and 1.0 μg of mRNA with both ADGN-100 and ADGN-106 particles. Level of P53 increased by 5 folds and 7-8 folds with 0.5 and 1.0 μg mRNA, respectively. Only a small change in P53 level was observed using 0.25 μg mRNA (1.2 fold the background).

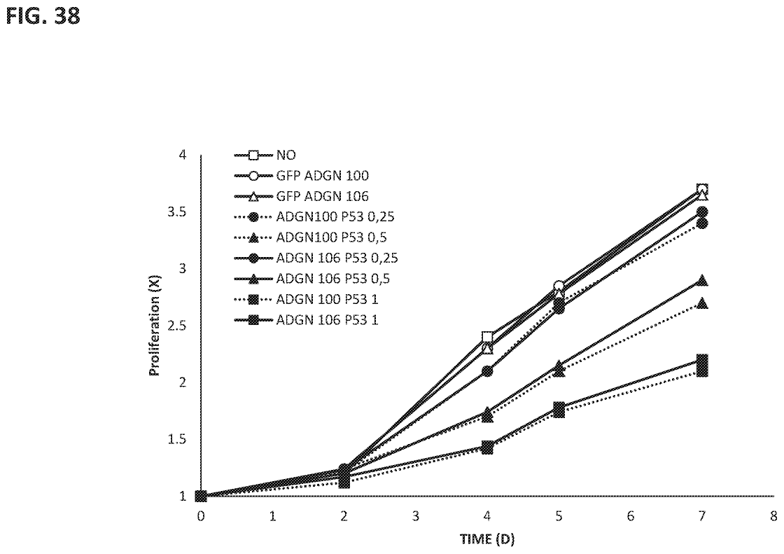

As reported in FIG. 38, expression of P53 WT mRNA directly correlated to an inhibition of cell proliferation. Cell proliferation inhibition of 59% and 56% were obtained using 1.0 μg mRNA complexed with ADGN-106 or ADGN-100, respectively. Cell proliferation inhibition of 31% and 38% were obtained using 0.5 μg mRNA complexed with ADGN-106 or ADGN-100, respectively. In contrast, only a slight inhibition of cell proliferation by 8% and 11% were obtained using 0.25 μg mRNA complexed with ADGN-106 or ADGN-100, respectively. The reduction in growth curve of G292 cells is marked at Day 7 and indicates that wild type P53 expression decrease the viability of the cells or slow down their proliferation. eGFP mRNA complexed with ADGN-106 or ADGN-100 were used as controls (0.5 ug) and showed no inhibition in cell proliferation similar to a no treatment control.

The results demonstrated that ADGN-100 and ADGN-106 are potent agents for the delivery of P53 mRNA in Human Osteosarcoma cell G-292. ADGN peptides mediated mRNA delivery leads to rescue P53 functions, by inhibiting the growth of tumor cells.

B. Delivery of 5moU Modified eGFP mRNA

ADGN-100/mRNA and ADGN-106/mRNA complexes were evaluated for cellular delivery of 5moU modified eGFP mRNA in Human Osteosarcoma cell G-292. We evaluated the impact of 5moU modification of the mRNA on the level of cellular expression and the stability of the ADGN/mRNA complexes. The G292 cells were grown in 24 well plates. 0.5 μg and 1 μg of eGFP mRNA unmodified and 5 moU modified were associated to ADGN-100 and ADGN-106 and the stability of ADGN/mRNA complexes was evaluated following 3 hr incubation in the presence of complete medium containing either 10% or 25% fetal calf serum (FCS) prior cell treatment. The level of eGFP expression was evaluated by flow cytometry at day 6.

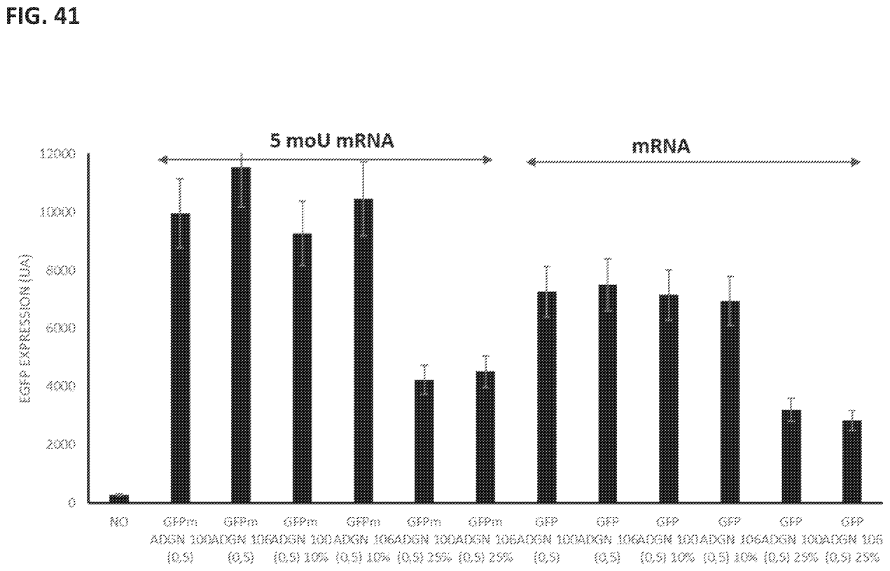

As reported in FIG. 41, in all the cases, ADGN-100 and ADGN-106 promoted efficient delivery of eGFP mRNA and of eGFP mRNA 5moU leading to eGFP expression. For both ADGN-100 and ADGN-106, the eGFP expression was increased by 25% when using mRNA including 5 moU modification. No modification on eGFP expression level was observed following incubation with 100% serum. In contrast, the presence of 25% serum decreased by 50-60% eGFP expression whatever the mRNA used.

### Example 18: ADGN Mediated in Vivo mRNA Luc Delivery Via Local Delivery

Stable ADGN-106/mRNA were evaluated for in vivo delivery of Luciferase mRNA via nebulization/non-surgical intratracheal administration. 5 moU modified Luc mRNA (60 μg) in sterile water (GIBCO) were mixed with ADGN peptide (sterile water), and the volume was adjusted to 500 μl with sterile water. Samples were mixed gently with vortex for 1 minute at low speed and incubated for 30 mins at room temperature. Just before the injection, volume was adjusted to 600 μl with sucrose 20% to reach a final sucrose concentration of 5%. Samples were mixed gently with vortex for 1 min at low speed and administrated into mice.

Mice from Group 1 received via nebulizationnon-surgical intratracheal administration of ADGN-106/mRNA solutions containing 10 μg of Luc mRNA respectively, (6 animals per group). As control, mice from group 2 (2 animals per group) received nebulization/non-surgical intratracheal administration of 100 μl saline solution. mRNA Luc expression was monitored by bioluminescence. Bioluminescence imaging was performed 6 hours and 24 hours after administration. Mice received an i.p. injection of 150 μg/g luciferin for non-invasive bioluminescence imaging (IVIS Kinetic; PerkinElmer, Waltham, Mass., USA). Animals were sacrificed at 24 h and organ harvested. mRNA expression in the different organs was monitored by bioluminescence. The organs are incubated with luciférine (300 μg/mL), then analysis by ex vivo bioluminescence.

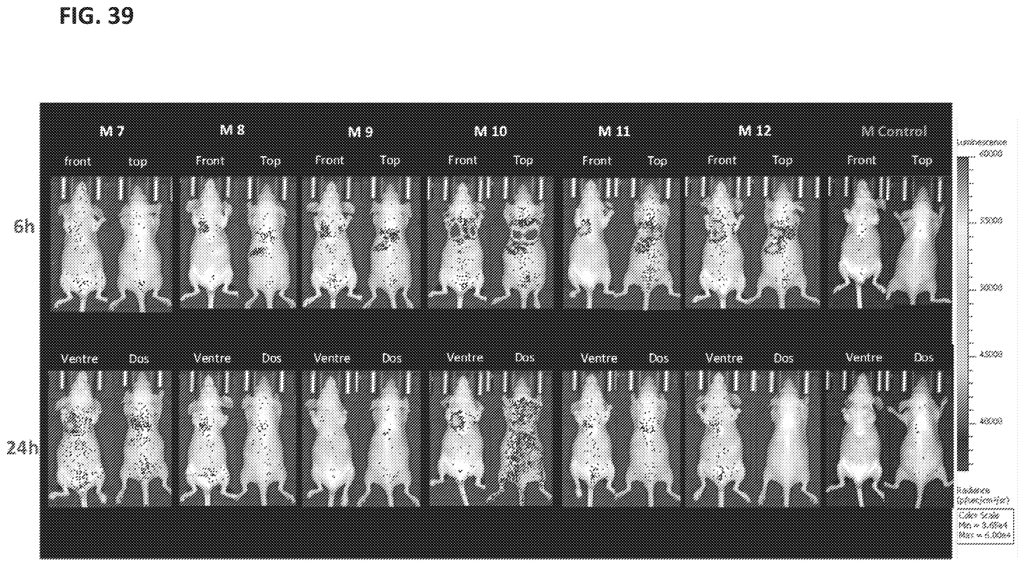

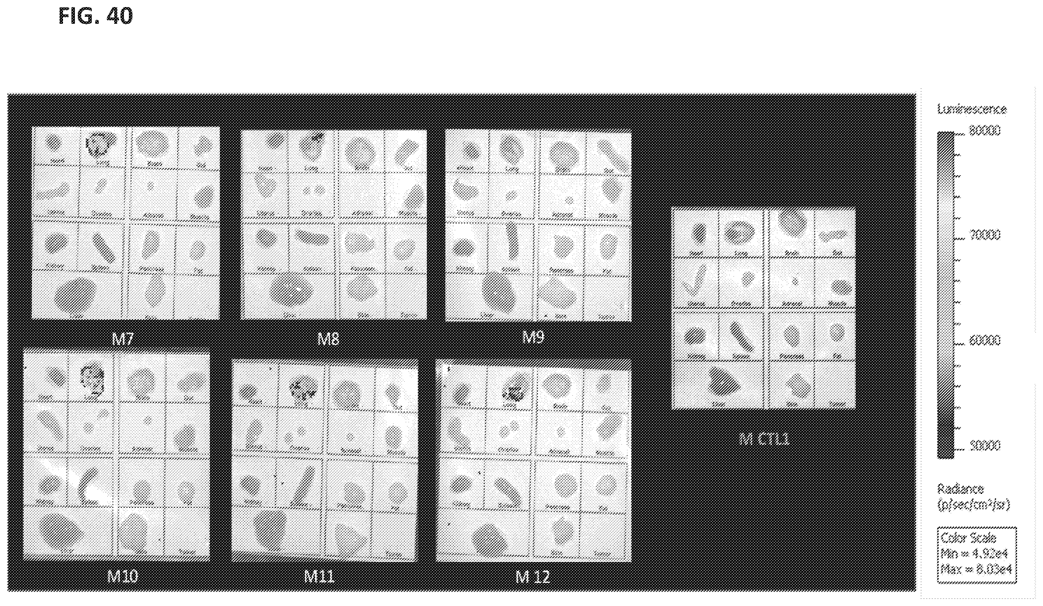

As reported in FIG. 39, ADGN-106 mediated in vivo mRNA delivery via nebulization. High luciferase expression was observed mainly in the lung after 6 hrs and remains at 24 hrs. In contrast no luciferase signal was observed in the control Mice group. As shown in FIG. 40, analysis of the different organs demonstrated that Luciferase expression is mainly in the lung after 24 hr.

The results demonstrated that ADGN-106 is a potent technology for in vivo targeted delivery of mRNA in the lung via nebulization/non-surgical intratracheal administration.

### Example 19: Nanoparticle-Mediated In Vivo Delivery of Functional mRNAs: Implication for Cancer Therapy

RNA is a universal molecule, comprising all biochemical functions of life and constituting a central player in all cellular processes. As such, mRNA therapeutics have myriads applications and offer several advantages. An exciting development for mRNA therapeutics in cancer therapy is to rescue a functional version of a protein that is mutated or missing.

However, efficient delivery of functionally intact mRNA into cells remains a key challenge in mRNA therapeutic field. The mRNA molecules are unable to cross cellular/tissue barriers, susceptible to rapid degradation by nucleases and activate innate immune pathways. Here we report a new delivery platform that can potently protect and deliver functional mRNAs in mammalian cell lines and in vivo.

ADGN-technology is based on short amphipathic peptides that form stable neutral nanoparticles with nucleic acid complexes through non-covalent electrostatic and hydrophobic interactions. Self-assembled of peptide/mRNA nanoparticles remain stable over time in serum and plasma conditions. We demonstrate the efficacy of ADGN-nanoparticles to complex, deliver, and release mRNA in multiple cell lines including primary T cells and in animal models. When applied by systemic intravenous injections. ADGN promotes the delivery of mRNA in the targeted tissues without triggering any nonspecific inflammatory response.

We investigated ADGN-technology for tumor suppressor rescue that may have therapeutic potential in cancers. The tumor suppressors PTEN and P53 play essential roles in tumorigenesis. P53 and PTEN mutations and resulting loss of function are common across variety of tumors and influence tumor cell proliferation. We showed that ADGN-mediated delivery of wt P53 mRNA or wt PTEN mRNA rescued tumor suppressor function in several cancer cell-lines including pancreas (PANC1), ovarian (SKOV3), prostate (PC3), glioblastoma (U25) and osteosarcoma (G292). Restoring PTEN resulted in reduction of cell proliferation, activation of cell apoptosis, reduction of AKT phosphorylation and cell cycle arrest in G1. In-vivo Efficacy of IV-administered ADGN/PTEN mRNA and ADGN/P53 mRNA were tested in PANC1 mouse xenografts. ADGN-nanoparticles containing wt p53 mRNA (0.5 mg/kg) and wt PTEN mRNA (0.5 mg/kg) resulted in tumor growth inhibition (TGI) of 50% and 80%, respectively. Combination ADGN-PTEN mRNA nanoparticles and ADGN-p53 mRNA nanoparticles, resulted in a TGI of 90% in PANC1 xenografts and also slowed development of distant metastases. No nonspecific cytokine response was observed following administration of ADGN-nucleic acid complexes.

ADGN-nanoparticles complexed with mRNAs were effective in rescuing PTEN and P53 both in vitro and in vivo. Considering the strong potential of mRNA therapy, this study sheds light on the potency of ADGN-mediated mRNA delivery for validating tumor suppressors as a therapeutic target in cancer treatment.

SEQUENCE LISTING

| SEQ ID | Sequence | Annotations |
|---|---|---|
| 1 | X₁X₂X₃X₄X₅X₂X₃X₄X₆X₇X₃X₈X₉X₁₀X₁₁X₁₂X₁₃ | VEPEP-3 |
| | X₁ is beta-A or S, X₂ is K, R or L, X₃ is F or W, X₄ is | |
| | F, W or Y, X₅ is E, R or S, X₆ is R, T or S, X₇ is E, R, | |
| | or S, X₈ is none, F or W, X₉ is P or R, X₁₀ is R or L, | |
| | X₁₁ is K, W or R, X₁₂ is R or F, and X₁₃ is R or K | |
| 2 | X₁X₂WX₄EX₂WX₄X₆X₇X₃PRX₁₁RX₁₃ | VEPEP-3 1 |
| | X₁ is beta-A or S, X₂ is R or K, X₃ is W or F, X₄ is F, | |
| | W, or Y, X₆ is T or R, X₇ is E or R, X₁₁ is R or K, and | |
| | X₁₃ is R or K | |
| 3 | X₁KWFERWFREWPRKRR | VEPEP-3 1a |
| | X₁ is beta-A or S | |
| 4 | X₁KWWERWWREWPRKRR | VEPEP-3 1b |
| | X₁ is beta-A or S | |
| 5 | X₁KWWERWWREWPRKRK | VEPEP-3 1c |
| | X₁ is beta-A or S | |
| 6 | X₁RWWEKWWTRWPRKRK | VEPEP-3 1d |
| | X₁ is beta-A or S | |
| 7 | X₁RWYEKWYTEFPRRRR | VEPEP-3 1e |
| | X₁ is beta-A or S | |
| 8 | X₁KX₁₄WWERWWRX₁₄WPRKRK | VEPEP-3 1S |
| | X₁ is beta-A or S and X₁₄ is a non-natural amino acid, | |
| | and wherein there is a hydrocarbon linkage between the | |
| | two non-natural amino acids | |
| 9 | X₁X₂X₃WX₅X₁₀X₃WX₆X₇WX₈X₉X₁₀WX₁₂R | VEPEP-3 2 |
| | X₁ is beta-A or S, X₂ is K, R or L, X₃ is F or W, X₅ is R | |
| | or S, X₆ is R or S, X₇ is R or S, X₈ is F or W, X₉ is R or | |
| | P, X₁₀ is L or R, and X₁₂ is R or F | |
| 10 | X₁RWWRLWWRSWFRLWRR | VEPEP-3 2a |
| | X₁ is beta-A or S | |
| 11 | X₁LWWRRWWSRWWPRWRR | VEPEP-3 2b |
| | X₁ is beta-A or S | |
| 12 | X₁LWWSRWWRSWFRLWFR | VEPEP-3 2c |
| | X₁ is beta-A or S | |
| 13 | X₁KFWSRFWRSWFRLWRR | VEPEP-3 2d |
| | X₁ is beta-A or S | |
| 14 | X₁RWWX₁₄LWWRSWX₁₄RLWRR | VEPEP-3 2S |
| | X₁ is a beta-alanine or a serine and X₁₄ is a non-natural | |
| | amino acid, and wherein there is a hydrocarbon linkage | |
| | between the two non-natural amino acids | |
| 15 | X₁LX₂RALWX₉LX₃X₉X₄LWX₉LX₅X₆X₇X₈ | VEPEP-6 1 |
| | X₁ is beta-A or S, X₂ is F or W, X₃ is L, W, C or I, X₄ | |
| | is S, A, N or T, X₅ is L or W, X₆ is W or R, X₇ is K or | |
| | R, X₈ is A or none, and X₉ is R or S | |
| 16 | X₁LX₂LARWX₉LX₃X₉X₄LWX₉LX₅X₆X₇X₈ | VEPEP-6 2 |
| | X₁ is beta-A or S, X₂ is F or W, X₃ is L, W, C or I, X₄ | |
| | is S, A, N or T, X₅ is L or W, X₆ is W or R, X₇ is K or | |
| | R, X₈ is A or none, and X₉ is R or S | |
| 17 | X₁LX₂ARLWX₉LX₃X₉X₄LWX₉LX₅X₆X₇X₈ | VEPEP-6 3 |
| | X₁ is beta-A or S, X₂ is F or W, X₃ is L, W, C or I, X₄ | |
| | is S, A, N or T, X₅ is L or W, X₆ is W or R, X₇ is K or | |
| | R, X₈ is A or none, and X₉ is R or S | |
| 18 | X₁LX₂RALWRLX₃RX₄LWRLX₅X₆X₇X₈ | VEPEP-6 4 |
| | X₁ is beta-A or S, X₂ is F or W, X₃ is L, W, C or I, X₄ | |
| | is S, A, N or T, X₅ is L or W, X₆ is W or R, X₇ is K or | |
| | R, and X₈ is A or none | |
| 19 | X₁LX₂RALWRLX₃RX₄LWRLX₅X₆KX₇ | VEPEP-6 5 |
| | X₁ is beta-A or S, X₂ is F or W, X₃ is L or W, X₄ is S, | |
| | A or N, X₅ is L or W, X₆ is W or R, X₇ is A or none | |
| 20 | X₁LFRALWRLLRX₂LWRLLWX₃ | VEPEP-6 6 |
| | X₁ is beta-A or S, X₂ is S or T, and X₃ is K or R | |
| 21 | X₁LWRALWRLWRX₂LWRLLWX₃A | VEPEP-6 7 |
| | X₁ is beta-A or S, X₂ is S or T, and X₃ is K or R | |
| 22 | X₁LWRALWRLX₄RX₂LWRLWRX₃A | VEPEP-6 8 |
| | X₁ is beta-A or S, X₂ is S or T, X₃ is K or R, and X₄ is | |
| | L, C or I | |
| 23 | X₁LWRALWRLWRX₂LWRLWRX₃A | VEPEP-6 9 |
| | X₁ is beta-A or S, X₂ is S or T, and X₃ is K or R | |
| 24 | X₁LWRALWRLX₅RALWRLLWX₃A | VEPEP-6 10 |
| | X₁ is beta-A or S, X₃ is K or R, and X₅ is L or I | |
| 25 | X₁LWRALWRLX₄RNLWRLLWX₃A | VEPEP-6 11 |
| | X₁ is beta-A or S, X₃ is K or R, and X₄ is L, C or I | |
| 26 | Ac-X₁LFRALWRLLRSLWRLLWK-cysteamide | VEPEP-6a |
| | X₁ is beta-A or S | |
| 27 | Ac-X₁LWRALWRLWRSLWRLLWKA-cysteamide | VEPEP-6b |
| | X₁ is beta-A or S | |
| 28 | Ac-X₁LWRALWRLLRSLWRLWRKA-cysteamide | VEPEP-6c |
| | X₁ is beta-A or S | |
| 29 | Ac-X₁LWRALWRLWRSLWRLWRKA-cysteamide | VEPEP-6d |
| | X₁ is beta-A or S | |
| 30 | Ac-X₁LWRALWRLLRALWRLLWKA-cysteamide | VEPEP-6e |
| | X₁ is beta-A or S | |
| 31 | Ac-X₁LWRALWRLLRNLWRLLWKA-cysteamide | VEPEP-6f |
| | X₁ is beta-A or S | |
| 32 | Ac-X₁LFRALWRₛLLRSₛLWRLLWK-cysteamide | ST-VEPEP-6a |
| | X₁ is beta-A or S and the residues followed by an | |
| | inferior “s” are linked by a hydrocarbon linkage | |
| 33 | Ac-X₁LFLARWRₛLLRSₛLWRLLWK-cysteamide | ST-VEPEP-6aa |
| | X₁ is beta-A or S and the residues followed by an | |
| | inferior “s” are linked by a hydrocarbon linkage | |
| 34 | Ac-X₁LFRALWSₛLLRSₛLWRLLWK-cysteamide | ST-VEPEP-6ab |
| | X₁ is beta-A or S and the residues followed by an | |
| | inferior “s” are linked by a hydrocarbon linkage | |
| 35 | Ac-X₁LFLARWSₛLLRSₛLWRLLWK-cysteamide | ST-VEPEP-6ad |
| | X₁ is beta-A or S and the residues followed by an | |
| | inferior “s” are linked by a hydrocarbon linkage | |
| 36 | Ac-X₁LFRALWRLLRₛSLWSₛLLWK-cysteamide | ST-VEPEP-6b |
| | X₁ is beta-A or S and the residues followed by an | |
| | inferior “s” are linked by a hydrocarbon linkage | |
| 37 | Ac-X₁LFLARWRLLRₛSLWSₛLLWK-cysteamide | ST-VEPEP-6ba |
| | X₁ is beta-A or S and the residues followed by an | |
| | inferior “s” are linked by a hydrocarbon linkage | |
| 38 | Ac-X₁LFRALWRLLSₛSLWSₛLLWK-cysteamide | ST-VEPEP-6bb |
| | X₁ is beta-A or S and the residues followed by an | |
| | inferior “s” are linked by a hydrocarbon linkage | |
| 39 | Ac-X₁LFLARWRLLSₛSLWSₛLLWK-cysteamide | ST-VEPEP-6bd |
| | X₁ is beta-A or S and the residues followed by an | |
| | inferior “s” are linked by a hydrocarbon linkage | |
| 40 | Ac-X₁LFARₛLWRLLRSₛLWRLLWK-cysteamide | ST-VEPEP-6c |
| | X₁ is beta-A or S and the residues followed by an | |
| | inferior “s” are linked by a hydrocarbon linkage | |
| 41 | X₁X₂X₃WWX₄X₅WAX₆X₃X₇X₈X₉X₁₀X₁₁X₁₂WX₁₃R | VEPEP-9 1 |
| | X₁ is beta-A or S, X₂ is L or none, X₃ is R or none, X₄ | |
| | is L, R or G, X₅ is R, W or S, X₆ is S, P or T, X₇ is W | |
| | or P, X₈ is F, A or R, X₉ is S, L, P or R, X₁₀ is R or S, | |
| | X₁₁ is W or none, X₁₂ is A, R or none and X₁₃ is W or | |
| | F, and wherein if X₃ is none, then X₂, X₁₁ and X₁₂ are | |
| | none as well | |
| 42 | X₁X₂RWWLRWAX₆RWX₈X₉X₁₀WX₁₂WX₁₃R | VEPEP-9 2 |
| | X₁ is beta-A or S, X₂ is L or none, X₆ is S or P, X₈ is F | |
| | or A, X₉ is S, L or P, X₁₀ is R or S, X₁₂ is A or R, and | |
| | X₁₃ is W or F | |
| 43 | X₁LRWWLRWASRWFSRWAWWR | VEPEP9a1 |
| | X₁ is beta-A or S | |
| 44 | X₁LRWWLRWASRWASRWAWFR | VEPEP9a2 |
| | X₁ is beta-A or S | |
| 45 | X₁RWWLRWASRWALSWRWWR | VEPEP9b1 |
| | X₁ is beta-A or S | |
| 46 | X₁RWWLRWASRWFLSWRWWR | VEPEP9b2 |
| | X₁ is beta-A or S | |
| 47 | X₁RWWLRWAPRWFPSWRWWR | VEPEP9c1 |
| | X₁ is beta-A or S | |
| 48 | X₁RWWLRWASRWAPSWRWWR | VEPEP9c2 |
| | X₁ is beta-A or S | |
| 49 | X₁WWX₄X₅WAX₆X₇X₈RX₁₀WWR | VEPEP-9 3 |
| | X₁ is beta-A or S, X₄ is R or G, X₅ is W or S, X₆ is S, T | |
| | or P, X₇ is W or P, X₈ is A or R, and X₁₀ is S or R | |
| 50 | X₁WWRWWASWARSWWR | VEPEP9d |
| | X₁ is beta-A or S | |
| 51 | X₁WWGSWATPRRRWWR | VEPEP9e |
| | X₁ is beta-A or S | |
| 52 | X₁WWRWWAPWARSWWR | VEPEP9f |
| | X₁ is beta-A or S | |
| 53 | X₁KWRSX₂X₃X₄RWRLWRX₅X₆X₇X₈SR | ADGN-100 |
| | X₁ is any amino acid or none, and X₂-X₈ are any amino | |
| | acid | |
| 54 | X₁KWRSX₂X₃X₄RWRLWRX₅X₆X₇X₈SR | ADGN-100 1 |
| | X₁ is βA, S, or none, X₂ is A or V, X₃ is G or L, X₄ is | |
| | W or Y, X₅ is V or S, X₆ is R, V, or A, X₇ is S or L, | |
| | and X₈ is W or Y | |
| 55 | KWRSAGWRWRLWRVRSWSR | ADGN-100a |
| 56 | KWRSALYRWRLWRVRSWSR | ADGN-100b |
| 57 | KWRSALYRWRLWRSRSWSR | ADGN-100c |
| 58 | KWRSALYRWRLWRSALYSR | ADGN-100d |
| 59 | KWRS_{S}AGWR_{S}WRLWRVRSWSR | ADGN-100 aa |
| | the residues marked with a subscript “S” are linked by | |
| | a hydrocarbon linkage | |
| 60 | KWR_{S}SAGWRWR_{S}LWRVRSWSR | ADGN-100 ab |
| | the residues marked with a subscript “S” are linked by | |
| | a hydrocarbon linkage | |
| 61 | KWRSAGWR_{S}WRLWRVR_{S}SWSR | ADGN-100 ac |
| | the residues marked with a subscript “S” are linked by | |
| | a hydrocarbon linkage | |
| 62 | KWRS_{S}ALYR_{S}WRLWRSRSWSR | ADGN-100 ba |
| | the residues marked with a subscript “S” are linked by | |
| | a hydrocarbon linkage | |
| 63 | KWR_{S}SALYRWR_{S}LWRSRSWSR | ADGN-100 bb |
| | the residues marked with a subscript “S” are linked by | |
| | a hydrocarbon linkage | |
| 64 | KWRSALYR_{S}WRLWRSR_{S}SWSR | ADGN-100 bc |
| | the residues marked with a subscript “S” are linked by | |
| | a hydrocarbon linkage | |
| 65 | KWRSALYRWR_{S}LWRS_{S}RSWSR | ADGN-100 bd |
| | the residues marked with a subscript “S” are linked by | |
| | a hydrocarbon linkage | |
| 66 | KWRSALYRWRLWRS_{S}RSWS_{S}R | ADGN-100 be |
| | the residues marked with a subscript “S” are linked by | |
| | a hydrocarbon linkage | |
| 67 | KWR_{S}SALYRWR_{S}LWRSALYSR | ADGN-100 ca |
| | the residues marked with a subscript “S” are linked by | |
| | a hydrocarbon linkage | |
| 68 | KWRS_{S}ALYR_{S}WRLWRSALYSR | ADGN-100 cb |
| | the residues marked with a subscript “S” are linked by | |
| | a hydrocarbon linkage | |
| 69 | KWRSALYRWR_{S}LWRS_{S}ALYSR | ADGN-100 cc |
| | the residues marked with a subscript “S” are linked by | |
| | a hydrocarbon linkage | |
| 70 | KWRSALYRWRLWRS_{S}ALYS_{S}R | ADGN-100 cd |
| | the residues marked with a subscript “S” are linked by | |
| | a hydrocarbon linkage | |
| 71 | KETWWETWWTEWSQPKKKRKV | PEP-1 |
| 72 | KETWFETWFTEWSQPKKKRKV | PEP-2 |
| 73 | KWFETWFTEWPKKRK | PEP-3 |
| 74 | GALFLGFLGAAGSTMGAWSQPKKKRKV | MPG |
| 75 | beta-AKWFERWFREWPRKRR | VEPEP-3a |
| 76 | beta-AKWWERWWREWPRKRR | VEPEP-3b |
| 77 | beta-ALWRALWRLWRSLWRLLWKA | VEPEP-6 |
| 78 | beta-ALRWWLRWASRWFSRWAWWR | VEPEP-9 |
| 79 | beta-AKWRSAGWRWRLWRVRSWSR | ADGN-100a |
| 80 | beta-AKWRSALYRWRLWRVRSWSR | ADGN-100b |
| 81 | GLWRALWRLLRSLWRLLWKV | CADY |
| 82 | ACAACTTTACCGACCGCGCC | Luciferase target site |
| 83 | 5′-GUUGGAGCUUGUGGCGUAGTT-3′ | KRAS siRNA targeting |
| | | G12C mutation (sense) |
| 84 | 5′-CUACGCCACCAGCUCCAACTT-3′ | KRAS siRNA targeting |
| | | G12C mutation (anti- |
| | | sense) |
| 85 | 5′-GATGAGGCTATTCATGATGATT-3′ | Factor VIII siRNA |
| | | (sense) |
| 86 | 5′-GAAGUGCAUACACCGAGACTT-3′ | KRAS siRNA targeting |
| | | Q61K mutation (sense) |
| 87 | 5′-GUCUCGGUGUAGCACUUCTT-3′ | KRAS siRNA targeting |
| | | Q61K mutation (anti- |
| | | sense) |
| 88 | 5′-GUUGGAGCUGUUGGCGUAGTT-3′ | KRAS siRNA targeting |
| | | G12D mutation (sense) |
| 89 | 5′-CUACGCCAACAGCUCCAACTT-3′ | KRAS siRNA targeting |
| | | G12D mutation (anti- |
| | | sense) |
| 90 | ATGAAGCACCTGAACACCGT | Factor VIII target site |
| 91 | CCRCCAUGG | Exemplary consensus |
| | R is a purine (adenine or guanine) | sequence in Kozak |
| | | sequences |
| 92 | (gcc)gccRccAUGG | Exemplary consensus |
| | R is a purine (adenine or guanine) | sequence in Kozak |
| | | sequences |

1. An mRNA delivery complex for intracellular delivery of an mRNA comprising a cell-penetrating peptide (CPP) and the mRNA, wherein the cell-penetrating peptide is selected from the group consisting of VEPEP-3 peptides, VEPEP-6 peptides, VEPEP-9 peptides, and ADGN-100 peptides.

2. (canceled)

3. An mRNA delivery complex for intracellular delivery of an mRNA comprising a cell-penetrating peptide (CPP) and the mRNA, wherein the mRNA encodes a therapeutic protein.

4. An mRNA delivery complex for intracellular delivery of an mRNA comprising a cell-penetrating peptide (CPP) and the mRNA, wherein the mRNA delivery complex further comprises an RNAi.

5. The mRNA delivery complex of claim 4, wherein the mRNA encodes a therapeutic protein for treating a disease or condition, and wherein the RNAi targets an RNA, wherein expression of the RNA is associated with the disease or condition.

6. The mRNA delivery complex of claim 1, wherein the cell-penetrating peptide is a VEPEP-6 peptide or an ADGN-100 peptide.

7. The mRNA delivery complex of claim 1, wherein the cell-penetrating peptide is covalently linked to the mRNA.

8. The mRNA delivery complex of claim 1, wherein the cell-penetrating peptide comprises an acetyl group covalently linked to its N-terminus.

9. The mRNA delivery complex of claim 1, wherein the cell-penetrating peptide comprises a cysteamide group covalently linked to its C-terminus.

10. The mRNA delivery complex of claim 1, wherein at least some of the cell-penetrating peptides in the mRNA delivery complex are linked to a targeting moiety by a linkage.

11. The mRNA delivery complex of claim 1, wherein the molar ratio of the cell-penetrating peptide to the mRNA is between about 1:1 and about 100:1.

12. (canceled)

13. A nanoparticle comprising a core comprising the mRNA delivery complex of claim 1.

14. (canceled)

15. The nanoparticle of claim 13, wherein the core further comprises an RNAi.

16. The nanoparticle of claim 15, wherein the RNAi targets an oncogene for downregulation.

17-18. (canceled)

19. A pharmaceutical composition comprising the mRNA delivery complex of claim 1, and a pharmaceutically acceptable carrier.

20. A method of preparing the mRNA delivery complex of claim 1, comprising combining the cell-penetrating peptide with the one or more mRNA, thereby forming the mRNA delivery complex.

21-24. (canceled)

25. A method of delivering one or more mRNA into a cell, comprising contacting the cell with the mRNA delivery complex of claim 1, wherein the mRNA delivery complex or the nanoparticle comprises the one or more mRNA.

26. A method of treating a disease in an individual comprising administering to the individual an effective amount of the pharmaceutical composition of claim 19.

27-31. (canceled)

32. A kit comprising a composition comprising the mRNA delivery complex of claim 1.

33. A method of treating a cancer in an individual comprising administering to the individual an effective amount of the mRNA delivery complex of claim 3, wherein the mRNA encodes a tumor suppressor protein, wherein the tumor suppressor protein corresponds to a tumor suppressor gene selected from PTEN, Retinoblastoma RB (or RB1), TP53, TP63, TP73, CDKN2A (INK4A), CDKN1B, CDKN1C, DLD/NP1, HEPACAM, SDHB, SDHD, SFRP1, TCF21, TIG1, MLH1, MSH2, MSH6, WT1, WT2, NF1, NF2N, VHL, KLF4, pVHL, APC, CD95, ST5, YPEL3, ST7, APC, MADR2, BRCA1, BRCA2, Patched, TSC1, TSC2, PALB2, ST14, or VHL.

34-40. (canceled)

41. A method of treating a disease or condition in an individual comprising administering an effective amount of the mRNA delivery complex of claim 1, wherein the mRNA encodes a therapeutic protein or a recombinant form thereof.
